SEQUENCE LISTING (811 sequences)

SEQ ID NO: 1 - DNA, 260 bp, Homo sapiens
aagttcccaggtgatctatcgggagaaccacgattgagaaccactgataaaggccgcctgtaggcatgtctgttggaggggaatgtgatgaaggtgcttttccgtttcattatcctcttttacaaaatctatttaaaaatataaacagaagcattaatcagtgtagctaattagtataatgaaatagaccatgcactaagcaggatttgtggaaatgaatattttggtacaaattgggattctcctctttaatcactgat

SEQ ID NO: 2 - DNA, 255 bp, Homo sapiens
atttgtgtatttgcacatggttgtgctgtcgaggacctggtgctgagaagagtctgttcacagccaaaattcttcccactgtcattcctaacctgggatttctagacacatcctgctgtgatgtaaacagaaatcacgaattcgctcactggatcaagttgttccactggtgtctaatacgctattgttgccggaggtgggttctgtgacgtgaagccatttcccatcattcaacagccagttacaattttctgt

SEQ ID NO: 3 - DNA, 239 bp, Homo sapiens
aagaacaactcctcaccagttcatcctgaggctgggaggaccgggatgctggattctgttttccgaagtcactgcagcggatgatggaactgaatcgatacggtgttttctgtccctcctactttccttcacaccagacagcccctcatgtctccaggacaggacaggactacagacaactctttctttaaataaattaagtctttacaataaaaacacaactgcaaagtaccttcata

SEQ ID NO: 4 - DNA, 244 bp, Homo sapiens
cactcccgggactattgccaagaaggggcaagggatgagtcaagaaggtgagacccttcccggtgggcacgtgggccaggctgtgtgagatgttggatgtttggtactgtccatgtctgggtgtgtgcctattacctcagcatttctcacaaagtgtaccatgtagcatgttttgtgtatataaaagggagggtttttttaaaaatatattcccagattatccttgtaatgacacgaatctgca

SEQ ID NO: 5 - DNA, 192 bp, Homo sapiens
agatcatccgccagcgggaacaacgcgttggagctctcccagctggagtaacagatcctggaaccaacagccgacatgttgcaccttgcccgcaagtacaaggaccctggagcacaagtccccgcccctgggcccattggcccccaacccaatcaaaaatctttcccccaccttgaggaagcactgcccaaa

SEQ ID NO: 6 - DNA, 214 bp, Homo sapiens
ggatcttgatgcctgaaaatcccaagattggtacttggcaaactgaaagaaatctagaaaaccctagagatcaggcatctgtggccagctaactggtcatacaaatggattgttgtggtgaacttgtatagtattaatcctgagatgctgtccccctccacccccacccccacaaaaaaaataaataaagtagtattaagttagcctcatacaa

SEQ ID NO: 7 - DNA, 164 bp, Homo sapiens
ccatggcaatgcgggtgactcctttacatggcacaacggcaagcagttcaccaccctggacagagatcatgatgtctacacaggaaactgtgcccactaccagaagggaggctggtggtataacgcctgtgcccactccaacctcaacggggtctggtaccgcg

SEQ ID NO: 8 - DNA, 225 bp, Homo sapiens
cccaaacctgtactgtcccggaggaggttgggaggtggaggcccagcatcccgcgcagatgacaccatcaaccgccagagtcccagacaccggttttcctagaagcccctcacccccactggcccactggtggctaggtctccccttatccttctggtccagcgcaaggaggggctgcttctgaggtcggtggctgtctttccattaaagaaacaccgtgcaacg

SEQ ID NO: 9 - DNA, 219 bp, Homo sapiens
ggcccggcccccatcacatgttcccttggcctcagagctgcccctgctctcccaccacagccacccagaggcaccccatgaagcttttttctcgttcactcccaaacccaagtgtccaaagctccagtcctaggagaacagtccctgggtcagcagccaggaggcggtccataagaatggggacagtgggctctgccagggctgccgcacctgtccaga

SEQ ID NO: 10 - DNA, 239 bp, Homo sapiens
gcaagagtgtttggcagacgttttttggatgtgctacaggctttcctcccttgttgcttatttttattgacaaacatccctaatgtgtgtcatgtctttctctgtctctccccttccctctctttttttctcaaattatgtaacttttgaggacagttacttctgtgataaactcttcaagtaagaccgttataagatagatttggaaattaaagccccagttgcatttcagtaactca

SEQ ID NO: 11 - DNA, 259 bp, Homo sapiens
ggtatccccagattagaatgtgtttatttgggttggagtatcactgactgtggcctctctgacttcaagtgcttggtgtaattcacatgccatctcctctgtgaagcctccttgtttctcccagtgaagcaggccacctggttctctttcctctcactccattttgtgcgtgctttcactctagcacttgccataccaaagggcaattatttatctacctatctgtatctccttgtgaaatttgtgctccacaaaccaa

SEQ ID NO: 12 - DNA, 175 bp, Homo sapiens
  misc_feature (76)..(76): n is a, c, g, or t
ccaccgcgcctagccagccaattttttttttttttgtatttttagtagagacagggttttaccatgttggccaggntggtcttgaactcctgacctcaagtgatctgcctgccatggcctcccaaagtgctgggattacaggtttgagccaccatgcctgggccaatatttaatt

SEQ ID NO: 13 - DNA, 224 bp, Homo sapiens
tttctcttgatgcaatgaagggaatatgacactacagtatacagagttgttgttatatgatgcacaaaatattttgatgatttgaataaatgttaacttttaatctgcgccttaataatgactggttatctgtaaatatagaacagatacagattgtatttttctgtgggtttttgtccttttagtgatttttttccaaaaacgagagatggaattaacattga

SEQ ID NO: 14 - DNA, 293 bp, Homo sapiens
tttgaatcgccaaatgcacgcacctctttcctggaaggtggagcgagtggaaggctaccccgtcagtatcactcagacattgctagtgtcagtggccgctggtagcagcaccctcttggcacatgcccgctgactaactgtaaagtggacacaggagatgtatgaacagccttcacagcacaccatccttagcactctgggtgtctggtatcaggaccaaagcattttattcgcacctgtactttatggcaaaaaggaagaagagagagaagatgttcttatgatgtcataca

SEQ ID NO: 15 - DNA, 122 bp, Homo sapiens
tctgttctgtcagtcatggcatggctggacagaattcagggacatctatcaggcctctcaggagcaattaaattattttcatgaaggcaatttgcagttgtaaacaatattgaagacactgt

SEQ ID NO: 16 - DNA, 175 bp, Homo sapiens
ctccccaatagctaggactatggttatgtgccactaggctcagctaatttttttaggttttgtagagacagtcttgccatgttgcccaggctagtgttgaacttctggcctcaagcgatcctcctgtctcggcctctcaaagcactgggattacagggtgtgaaccactatgcct

SEQ ID NO: 17 - DNA, 224 bp, Homo sapiens
tcagtgcacgatgctccagccacactggccatctttcggttcctgatacaaaaaaaaacacgttccttttccatggaaagcaggtcacccttgttattttgtatcgatgacaactctttaaacttattttgctttttggctttatgtatgtgtgtgggtgggtgggactgactgccccactagaatgtaagctccatgagggcagggaatcttgctttcttgtt

SEQ ID NO: 18 - DNA, 267 bp, Homo sapiens
ggaggcagacaatgaccccacggctcctccttatgactccattcaaatctacggttatgaaggcaggggctcagtggccgggtccctgagctccctagagtcggccaccacagattcagacttggactatgattatctacagaactggggacctcgttttaagaaactagcagatttgtatggttccaaagacacttttgatgacgattcttaacaataacgatacaaatttggccttaagaactgtgtctggcgttctcaagaatc

SEQ ID NO: 19 - DNA, 257 bp, Homo sapiens
ttgttactgctgattcttgtaaatctttttgcttctactttcatcttaaactaatacgtgccagatataactgtcttgtttcagtgagagacgccctatttctatgtcatttttaatgtatctatttgtacaattttaaagttcttattttagtatacgtataaatatcagtattctgacatgtaagaaaatgttacggcatcacacttatattttatgaacattgtactgttgctttaatatgagcttcaatataa

SEQ ID NO: 20 - DNA, 256 bp, Homo sapiens
actgcattgctggaccctctgtgaaactgaagccttctctacttgtttttatctcaagtgaacctggagaagcaacaataatggaccttctcccctagtcaaatagcctgtggacctcccctcatagtcagtctccaaaaacatgtatctggaattaatttattacaaagagaagtttagtgtttcttcttttttacatgcgctcaatactgactactggccagacacagcaccatcctcttacaaacatcatttc

SEQ ID NO: 21 - DNA, 260 bp, Homo sapiens
cttgaacatgggaaggagttgttgcagtaagccaagatgtgccactgtactccagcctgggagacagagtgagactctgtctcaaaaaatgaatgaataaataaataaataaataataaaaaagatgattattaacaatgccagttaatattaacaatattaatagtattatttattactaatgccattttttcatttttgttaaagtatttttattattttagtttaaattattattatgagacaaaggcctccatttc

SEQ ID NO: 22 - DNA, 273 bp, Homo sapiens
ccttgaaaagcagtgtcagagcagaattataaggcatttttaatattccctgttttaataaaactttttttatgtttgatttttttttatattttttgtccgcacgtatatagatgtggatacataacatttaacacggttgcaatcagagggtgatttgatttgttaacttaatgtcacatcataaacattttacatgctgttatataatgtacataatcatttttaatgactacataacatcccatcctattgacgaatcattatgtcctt

SEQ ID NO: 23 - DNA, 240 bp, Homo sapiens
  misc_feature (28)..(28): n is a, c, g, or t
  misc_feature (163)..(163): n is a, c, g, or t
aagtatacagctgtaagtaaccctgtcnccatggatgatccttttctctaggaatgtatttggattagagatgacaactacattttcgcatttttatgttgaagtcttttttaaaaaggctgtttacttttcagtagttaagaatacttgtttttctttttcnttttttttttttttaaccttttattttttcgttaagcctctattgtttgtagaacactcttagaaacttggaaataa

SEQ ID NO: 24 - DNA, 221 bp, Homo sapiens
ggcagccctgacgtgatgagctcaaccagcagagacattccatcccaagagaggtctgcgtgacgcgtccgggaggccaccctcagcaagaccaccgtacaattggtggaaggggtgacagctgcattctcctgtgcctaccacgtaaccaaaaatgaaggagaactactgtttacaagccgccctggtgtgcctgggcatgctgtgccacagccatgcct

SEQ ID NO: 25 - DNA, 129 bp, Homo sapiens
gatccagaactgtggctgggcaccgtggctcacatctgtaatctcatcactttgggaaggctaaggcgggtggatcacctaaggtaaggagttcgaacccagcctttacaatgtaatgaaaccctgcct

SEQ ID NO: 26 - DNA, 94 bp, Homo sapiens
gcctcccattcaagtgaagttataatttacactgagggtttcaaaattcgactagaagtggagatatattatttatttatgcactgtactgtat

SEQ ID NO: 27 - DNA, 129 bp, Homo sapiens
aatgcatatggaggtaggctgaaaagaatgtaatttttattttctgaaatacagatttgagctatcagaccaacaaaccttccccctgaaaagtgagcagcaacgtaaaaacgtatgtgaagcctctct

SEQ ID NO: 28 - DNA, 115 bp, Homo sapiens
tcatccatccaggatgtactaaaacagtgtgtttaataaattgtaattattttgtgtacagttttatactgttatctgtgtccatttccaaaacttgcacgtgtccctgaattcc

SEQ ID NO: 29 - DNA, 176 bp, Homo sapiens
gttaaattaattgataccagatttcactggaacagtttcaactgataatttatgacaaaagaacatacctgtaatattgaaattaaaaagtgaaatttgtcataaagaatttcttttatttttgaaatcgagtttgtaaatgtccttttaagaagggagatatgaatccaataaat

SEQ ID NO: 30 - DNA, 252 bp, Homo sapiens
tttcaactttccttttcctatttgaatttctttggtgctgtagaaaacaaaaaaagaaaaatatatattcataaaaaatatggtgctcattttcatccatccaggatgtactaaaacagtgtgtttaataaattgtaattattttgtgtacagttttatactgttatctgtgtccatttccaaaacttgcacgtgtccctgaattccatttgactttaattttatgagaattgcagaactttgatggcaata

SEQ ID NO: 31 - DNA, 230 bp, Homo sapiens
tacaaccatgacatttcaacttttgatatgggcaactaacagtgacccatctgtcagtcagaagtcctcgtgggtccctctgtctgtctgctaaaagtcctcatgggtccgtctgtctgtgttctaaatgaagtacttcttttctggcctactgcaattttacaaattaccttgtagagattagatacatttgtcccctctagacagaggtaacacctgagtacagacct

SEQ ID NO: 32 - DNA, 280 bp, Homo sapiens
ttaaaaattaggcaaagctgccagggtgtggtggctcgcacatgtaatcccaacactttgggagaccaaggcgggtggatcacctcaggtcgggagttcgagaccagcctgatcaacatggagaaaacccgtctttaccaaatatacaaaattacccaggcatggtggtgcatgcctgtaatcctagctactcgggaggctgaggcaggagaatcacttgaacccaggaggtggaggttgctgtgaaccaagattgcgcccttgcacaacaagagtgaaa

SEQ ID NO: 33 - DNA, 211 bp, Homo sapiens
gaaaaaactttctctttgccatttcttcttcttcttttttaactgaaagctgaatccttccatttcttctgcacatctacttgcttaaattgtgggcaaaagagaaaaagaaggattgatcagagcattgtgcaatacagtttcattaactccttccctcgctcccccaaaaatttgaatttttttttcaacactcttacacctgttatgg

SEQ ID NO: 34 - DNA, 130 bp, Homo sapiens
tcttttttcagaatctattaaggacacttgaaagttttgaaatttttggtaaatttggactaccatgaggaaacttttgagattcaagttcattctattcagagcaattccgatattgatgttaacttga

SEQ ID NO: 35 - DNA, 233 bp, Homo sapiens
caattttgtgcctaaattgcacattagaagatggattgattggacacatccatgtaattcaaagttattattcaaatttgacttaattggtaatcattgaaaaaactgactaatgtcatttagtgtgaaggagcactggccagctatatgccacactcatacatatgcattttcagaatgtgagcagcttttctgaatttttaatcaaaccttttcaccaactttactgaatg

SEQ ID NO: 36 - DNA, 152 bp, Homo sapiens
gtaaatcactgtaaacgtgtcttcatttactctagccaaaaggcctggcttctgataggaaactggtaagaaactcttcatgaaaacacatcactaatattcgctattactctcctggtctgaagtcagcttttctgaaccattaaggtatt

SEQ ID NO: 37 - DNA, 175 bp, Homo sapiens
gagttgtatcgtgtggtgtattttttaaaaaatttgatttagcattcatattttccattttattcccaattaaaagtatgcagattatttgcccaaagttgtcctttttttcagattcagcatttgttttttgccagtttcattttcatttttttccatggttccacagaagctt

SEQ ID NO: 38 - DNA, 226 bp, Homo sapiens
taaaggtggtttggcctctaatttaattttgattcagactctcctgtcaggactcaagaaaatttaattaattaccaaggattaagttttttggttaaggtttttgggaaaaaaaaatagcaaagatgttgatttcttggaatccttttacaggttcataacagaaaaatcttcattccctgtaggcatttaattaaacctagttgagaagtgtgtgggattcctc

SEQ ID NO: 39 - DNA, 270 bp, Homo sapiens
ctgttcctttcctgggttccaattttgtgcctaaattgcacattagaagatggattgattggacacatccatgtaattcaaagttattattcaaatttgacttaattggtaatcattgaaaaaactgactaatgtcatttagtgtgaaggagcactggccagctatatgccacactcatacatatgcattttcagaatgtgagcagcttttctgaatttttaatcaaaccttttcaccaactttactgaatgcctactggaattccataa

SEQ ID NO: 40 - DNA, 215 bp, Homo sapiens
caaggccctgctgtaagtatgatttggggaaataataaagaagatcacggacctaggaatgttttcttcagactaaaccaagacaactttgacaacccattaagttagccccatttcaatatatcctctaaaatatctggaaattgtctaaatgcaatgggctgtaagtccatccctgcagtgggcctgggggctcgttatttatttatggtgaa

SEQ ID NO: 41 - DNA, 181 bp, Homo sapiens
gagttgtatcgtgtggtgtattttttaaaaaatttgatttagcattcatattttccatcttattcccaattaaaagtatgcagattatttgcccaaatcttcttcagattcagcatttgttctttgccagtctcattttcatcttcttccatggttccacagaagctttgtttcttgggca

SEQ ID NO: 42 - DNA, 225 bp, Homo sapiens
ttttctgcacatttacttgcttaaattgtgggcaaaagagaaaaagaaggattgatcagagcattgtgcaatacagtttcattaactccttccctcgctcccccaaaaatttgaatttttttttcaacactcttacacctgttatggaaaatgtcaacctttgtaagaaaaccaaaataaaaattgaaaaataaaaaccataaacatttgccaactttcttgtac

SEQ ID NO: 43 - DNA, 211 bp, Homo sapiens
gaaaaaactttctctttgccatttcttcttcttcttttttaactgaaagctgaatccttccatttcttctgcacatctacttgcttaaattgtgggcaaaagagaaaaagaaggattgatcagagcattgtgcaatacagtttcattaactccttcccccgctcccccaaaaatttgaatttttttttcaacactcttacacctgttatgg

SEQ ID NO: 44 - DNA, 68 bp, Homo sapiens
ctggaaaagatggtcgcactggacatcctggtacagttggacctgctggcattcgaggccctcagggt

SEQ ID NO: 45 - DNA, 83 bp, Homo sapiens
taataacatggcacgatgaatgcttctttagagtaaaaaggttttctttaacttgttaagtcagagttgtctaagtaattgta

SEQ ID NO: 46 - DNA, 275 bp, Homo sapiens
gttatggtgctaatgtactttcacttttaaactctagatcagaattgttgacttgcattcagaacataaatgcacaaaatctgtacatgtctcccatcagaaagattcattggcatgccacaggggattttcctccttcatcctgtaaaggtcaacaataaaaaccaaattatggggctgcttttgtcacactagcatagagaatgtgttgaaatttaactttgtaagcttgtatgtggttgttgatcttttttttccttacagacacccataat

SEQ ID NO: 47 - DNA, 65 bp, Homo sapiens
ttaactccatatgtgttcctcttgttttaattttgtcaaccagtgcaagtgaccgacaaaattcc

SEQ ID NO: 48 - DNA, 286 bp, Homo sapiens
taaagacgcatgttatggtgctaatgtactttcacttttaaactctagatcagaattgttgacttgcattcagaacataaatgcacaaaatctgtacatgtctcccatcagaaagattcattggcatgccacaggggattctcctccttcatcctgtaaaggtcaacaataaaaaccaaattatggggctgcttttgtcacactagcatagagaatgtgttgaaatttaactttgtaagcttgtatgtggttgttgatcttttttttccttacagacacccataat

SEQ ID NO: 49 - DNA, 143 bp, Homo sapiens
tctgttgttagattgctgtctaattacattgcacaaccttgcctaagtttttatatacatagcattgagcttaaaaacatggaaccagctcttatgcataatttagctatatttagcatctttggtccaaagccttataaaaa

SEQ ID NO: 50 - DNA, 273 bp, Homo sapiens
aatgaccaccgccattcacaagaactttgactgtttgaagttgatcctgagactcttgaagtaatggctgatcctgcatcagcattgtatatatggtcttaagtgcctggcctccttatccttcagaatatttattttacttacaatcctcaagttttaattgattttaaatatttttcaatacaacagtttaggtttaagatgaccaatgacaatgaccacctttgcagaaagtaaactgattgaataaataaatctccgttttcttcaatt

SEQ ID NO: 51 - DNA, 193 bp, Homo sapiens
ctaaactggttatattttgacctgtatatcttaaatttgagtatttatatgcctaaatacatgtgtgagttttgtttgacttccaagtccaaactataagattatataagttcatatagatgaatcagaaatatgtggtaatactattaagtcacaaacactaacaatttccaactatagaaataacagttct

SEQ ID NO: 52 - DNA, 207 bp, Homo sapiens
agtttgtgactgatatttcaagcaaaatatattttcttcatgtttcaaaattaagtgctttttctctgttgttagattgctgtctaattacattgcacaaccttgcctaagtttttatatacatagcattgagcttaaaaacatggaaccagctcttatgcataatttagctatatttagcatctttggtccaaagccttataaaaa

SEQ ID NO: 53 - DNA, 270 bp, Homo sapiens
gaaagagcatcgttccaatgcttgttcactgttcctctgtcatactgtatctggaatgctttgtaatacttgcatgcttcttagaccagaacatgtaggtccccttgtgtctcaatactttttttttcttaattgcatttgttggctctattttaatttttttcttttaaaataaacagctgggaccatcccaaaagacaagccatgcatacaactttggtcatgtatctctgcaaagcatcaaattaaatgcacgcttttgtcatgtca

SEQ ID NO: 54 - DNA, 269 bp, Homo sapiens
tataaaaatttatgtaaggccgggcatggtggctcacgcctgtaatcccagcacttagggaggccagggtaggtggattacctgaggtcaggagttcaagaccagcctggccaacatggtgaaaccctgtctctattaaaaatacaaaaattagcctggcatggtggcatgtgcctgtaatcctagctattcaggaggctgaggtaggagaatcacttgaacccgggaggcggaggttgtagtgaaccaaaattacaccgttgcactcc

SEQ ID NO: 55 - DNA, 226 bp, Homo sapiens
tcactgccaattccagcacgaagttgggcgactgccaggcaggcactcccagtcgtcaaaaagtgcaaatgttactcagggaacaattaatgtgagttgtgtaatgtaatatgggtcaaaaacatgaaaagacgtttaaaatgtcagcggatggctcagcccacccatcagccagccagagagcagaacacctgttttgcactcagtggcacagaagccacaattt

SEQ ID NO: 56 - DNA, 263 bp, Homo sapiens
tcaggcccggagaccgggtgttcctccagatgccctcagaacaggctgcaggactgtatgccgggcagtatgtccactcctccttttcaggatatttattgtatcccatgtaaaaacaaaaaaacaaaaaacaaagaaaagaaagagattttatagaagaaaatgacacaccaaaaaatccaaatgaaaaacataattgcttcaaaacacttacacagttggaaagttatatgtaagtgaaaatttggaccattgtgtacaaa

SEQ ID NO: 57 - DNA, 245 bp, Homo sapiens
attgtgctataatccctatttagttcaaaattaaccagaatttttccatgtgaaatggaccaaactcatattattgttatgtaaatacagagttttaatgcagtatgacatcccacaggggaaaagaatgtctgtagtgggtgactgttatcaaatattttatagaatacaatgaacggtgaacagactggtaacttgtttgagttcccatgacagatttgagacttgtcaatagcaaatcattt

SEQ ID NO: 58 - DNA, 212 bp, Homo sapiens
  misc_feature (125)..(125): n is a, c, g, or t
acaggccagattatacaatacttctgtgcacaccagatggttgtaaagaaagatcagcgagaaaggctttggaagttttaatccttctttccaccttttttcccccgcatttagtaaatcacaancctacctgactggcatccaattatcagagatattcagtgtttaagctaccctctttaaaagaaaatgatctcttcttattcctaagg

SEQ ID NO: 59 - DNA, 221 bp, Homo sapiens
taatgtcatcctgtactcggcacaaatcaaaggccaatacaagtctgaaaagcagaaataaatatttttccaggtttttgctcgggcacatactaactgctttgggcattttaatctggtctccaaacaccaaagacccatttcgagcctgctattagcctgctgctgactctatcacttggagcaataatgtggggttatggtggtggaatcttgtatat

SEQ ID NO: 60 - DNA, 248 bp, Homo sapiens
aggctgtggattcaaggctccctgccccccagatcatttccccaatcctggcaaaagcccaaagatcccagggtcaggagagacccctctgtatccccaggtccctcccagaactgactcctaaggtctccagccagggcttttgagatgcaaaggtttggcctcaggagagtcaccttttctcacggccctggccttaactcatattttaggcattcctggccccagggccctaataaacctgcttt

SEQ ID NO: 61 - DNA, 229 bp, Homo sapiens
agccaaacaagagactaacctgatgtcaaactatgggagactgttgaataggcagggtttggggaaaatccagtttggtctccaatgcccaggggaagcactggcacagatgctggccctctctggaaggacttcagacagtcccaccagccccacatgtcctgctcttagggctcaattccaacacaagctagatgcgtgacctgggtcaattactgaccctccccga

SEQ ID NO: 62 - DNA, 222 bp, Homo sapiens
tttcacataggttagattctcattcacgggactagttagctttaagcaccctagaggactagggtaatctgacttctcacttcctaagttcccttctatatcctcaaggtagaaatgtctatgttttttactccaattcataaatctattcataagtctttggtacaagtttacatgataaaaagaaatgtgatttgtcttcccttctttgcacttttgaaa

SEQ ID NO: 63 - DNA, 32 bp, Homo sapiens
tgaggactcagaagttcaagctaaatattgtt

SEQ ID NO: 64 - DNA, 72 bp, Homo sapiens
gtgcccagcctatatctacttgagcctgtgtgcccactgaagagatgaagaataaaagcctaagctctcatc

SEQ ID NO: 65 - DNA, 162 bp, Homo sapiens
gattgctaaagctctggttaatgcacaagtggatttccaggcaatgtggtactctgaccagaaccacggcttatccggcctgtccacgaaccacttatacacccacatgacccacttcctaaagcagtgtttctctttgtcagactaaaaacgatgcagatg

SEQ ID NO: 66 - DNA, 112 bp, Homo sapiens
ttacaggtaattaattgttctcttcacttctcatggggcagcacagaaaggaataagttaggtaactgaagtgaccagccctcgaataaaaagtggcttcatggccgggtgt

SEQ ID NO: 67 - DNA, 145 bp, Homo sapiens
gataccgactgaccgtgggccttacccgaagaggacagcccatgcagtacaatgtgggtccctctgtcttcaagtacccactgaggaatctgcagcctgcatctgagtacaccgtattcctcgtggccataaagggcaaccaaga

SEQ ID NO: 68 - DNA, 269 bp, Homo sapiens
atcactaactacaaaatccgccatcatcccgagcacttcaatgggaaacctcgagaaaatcgggtgccccactctcggaattccatcaccctcaccaacctcactccaggcacagagtatgtggtcagcatcgttgcttttaatggcagagaggaaagtcccttattgattggccaacaatcaacaggtaacttttcttgtctgcaaagaaactcagaagactttcctacccagttggtagattctgtaaagtagcttgctgttgtctg

SEQ ID NO: 69 - DNA, 238 bp, Homo sapiens
tatatcaatgatggcaaaaaggttaaagggggcctaacagtactgtgtgtagtgttttatttttaacagtagtacactataacttaaaatagacttagattagactgtttgcatgattatgattctgtttcctttatgcatgaaatattgattttacctttccagctacttcgttagctttaattttaaaatacattaactgagtcttccttcttgttcgaaaccagctgttcctcct

SEQ ID NO: 70 - DNA, 241 bp, Homo sapiens
gcattttttctgtagtcttgtccaaaattgggtaaccacttctgatggggtagctcatatccaagaatgagtcacaaaaccagactcgttgaacctggtatatgatgagtcacaaagcaacattctgcctttgttttttcaggacaagaaacttgaattgtatcccactgagttaaaagataaaatatatggcattggcatttctgtacttcagagaggaatatatctgtttgtggtagga

SEQ ID NO: 71 - DNA, 69 bp, Homo sapiens
tcatttcatttgactattctgatgacatgattgtggcagaataaattgggtcttaaaatgccctagaaa

SEQ ID NO: 72 - DNA, 129 bp, Homo sapiens
gtctcctaatcttatcaattctgatggtttctttttttcccagcttttgagccaacaactctgattaactattcctatagcatttactatatttgtttagtgaacaaacaatatgtggtcaattaaatt

SEQ ID NO: 73 - DNA, 233 bp, Homo sapiens
tctgtagtcttgtccaaaattgggtaaccacttctgatggggtagctcatatccaagaatgagtcacaaaaccagactcgttgaacctggtatatgatgagtcacaaagcaacattctgcctttgttttttcaggacaagaaacttgaattgtatcccactgagttaaaagataaaatatatggcattggcatttctgtacttcagagaggaatatatctgtttgtggtagga

SEQ ID NO: 74 - DNA, 51 bp, Homo sapiens
ttgcaatgggagacttaaaagtggtataaaatgtactttgggccaggcgca

SEQ ID NO: 75 - DNA, 169 bp, Homo sapiens
gaccacatcgagcggtgaggcacaggacgagcaggggcgggaaatggggaagcaggtcaagaaatatttccgcaaatccatctttcctttgacatgccatttgaggataatttgcagtgtttcagctaataacctaagataatttacactattattggttgttaaaact

SEQ ID NO: 76 - DNA, 186 bp, Homo sapiens
tattcctgcaccaactgacctgaagttcactcaggtcacacccacaagcctgagcgcccagtggacaccacccaatgttcagctcactggatatcgagtgcgggtgacccccaaggagaagaccggaccaatgaaagaagtcaaccttgctcctgacagctcatccgtggttgtatcaggacttat

SEQ ID NO: 77 - DNA, 251 bp, Homo sapiens
gaggctaagccgggagcactgattgaggccaggagttcatgatcagcctgggcaatgaagtgagaccccgtctctacaaaaaaatatgaaaaaattagcgaggtgtggtgacacatgcctgtagtcccagctactcaagaggctgaggtagaggatcacttgagcctacgagttcaaggctgcagtgagctatgataactccactgcactgccgcctggatgacacagagagaccgtttctaaattaatta

SEQ ID NO: 78 - DNA, 122 bp, Homo sapiens
ggaacccacctagcccaacaagaacaatccattctacttcttggaactacgtttattttccttttcccccatttcctataagataacctctaaccaatgacaatctcgacagctattcctgc

SEQ ID NO: 79 - DNA, 54 bp, Homo sapiens
tgttgaaaatgtagaaaattggccggacggggtggctcacgtcagtaatttcag

SEQ ID NO: 80 - DNA, 196 bp, Homo sapiens
tctggcccgcaatactgtaggaacaagcatgatcttgttactgtgatattttaaatatccacagtactcactttttccaaatgatcctagtaattgcctagaaatatctttttcttacctgttatttatcaatttttcccagtatttttatacggaaaaaattgtattgaaaacacttagtatgcagttgataaga

SEQ ID NO: 81 - DNA, 248 bp, Homo sapiens
agttgctacaaaaactgattggtttttgtcacttcatctcttcactaatggagatagctttacactttctgctttaatagatttaagtggaccccaatatttattaaaattgctagtttaccgttcagaagtataatagaaataatctttagttgctcttttctaaccattgtaattcttcccttcttccctccacctttccttcattgaataaacctctgttcaaagagattgcctgcaagggaaat

SEQ ID NO: 82 - DNA, 291 bp, Homo sapiens
tcaaaccaccatcactaactacaaaatccgccatcatcccgagcacttcaatgggaaacctcgagaaaatcgggtgccccactctcggaattccatcaccctcaccaacctcactccaggcacagagtatgtggtcagcatcgttgcttttaatggcagagaggaaagtcccttattgattggccaacaatcaacaggtaacttttcttgtctgcaaagaaactcagaagactttcctacccagttggtagattctgtaaagtagcttgctgttgtctgtcatcagctctc

SEQ ID NO: 83 - DNA, 209 bp, Homo sapiens
gggcctaacagtactgtgtgtagtgttttatttttaacagtagtacactataacttaaaatagacttagattagactgtttgcatgattatgattctgtttcctttatgcatgaaatattgattttacctttccagctacttcgttagctttaattttaaaatacattaactgagtcttccttcttgttcgaaaccagctgttcctcct

SEQ ID NO: 84 - DNA, 129 bp, Homo sapiens
aggacagctgctgaaggcaccctctgatgagctcggttactcagaagagtgaggatgtgttgaaggtatctgctgtatggagtggcaggatgatgtctgtgattgagaaatataatcccggccaggcga

SEQ ID NO: 85 - DNA, 89 bp, Homo sapiens
aaagtctacccttaaaccctcagatcagtctttccaaagaattactctgtttgcattgttgtgattgacatttgtgaagtcccaagaaa

SEQ ID NO: 86 - DNA, 123 bp, Homo sapiens
aatttatcaaaatgccggtacttaggacctaaatttatctatgtctgtcatacgctaaaatgatattggtctttgaatttggtatacatttattctgttcactatcacaaaatcatctatatt

SEQ ID NO: 87 - DNA, 233 bp, Homo sapiens
aagaccctcaactcctggaggaagttctacacgaggctcaccaacagcaaacaaggggagactacagtctgagacccggggctcagcccatgcccaggcctcggccggggcgcaacgatcccccaaagccagcgccgtggagttcgtgccaatcctgacatctcgaggtttcctcactaacaactctctttcgcaggctcctttgaacaactcagctcctgcaaaagcttccg

SEQ ID NO: 88 - DNA, 204 bp, Homo sapiens
tacattttatggtgtttcatagccaatcccacagtgtaaaaattcaggaattcaatgaaaaaagtctacccttaaaccctcagatcagtctttccaaagaattactctgtttgcattgttgtgattgacatttgtgaagtcccaagaaaagatctgttttcatgacagtagaaaatagaagtttgcaaattatttctttactca

SEQ ID NO: 89 - DNA, 240 bp, Homo sapiens
ttctctggaagaactagcagatcggggcatgctgtgcagccctctctcaggtggcaggtgtctggatctgctcaaaggctgccccctttagatgagacaactgtgtctagacctcagtccctcccatgcccctccctgcctttgcctggggtgagaccatttgtgaaagacagagccaagacacagggagtttttcaattgattctaaagtccatgctctcatcaatccactgaataatg

SEQ ID NO: 90 - DNA, 296 bp, Homo sapiens
cagcggctgctggaacgcgttgaccctcactgtttgtgtgttttcaggaaggtgcattcgcgctggttttcaagagtgtccactacccaggagaagccgttgccacacggtgaggcaaaacacactggcatttcccataagaaagaacgaaaataaaatcacttagctggagggagacgggagaggtagggcctgtggcacgctgaaaggtgctcgtggtgtctaaagaacaactgtggctcagccccggggaccgcgccccaccatctccatggctggtggtgctacacttttga

SEQ ID NO: 91 - DNA, 238 bp, Homo sapiens
acctatgcctcctataagtccagttgaaatctcagcctccttcaacattttcttctcgtgtgtggcccacatccctccacttctccaacttctgtttaatctgatcacggctctttttaagccctggcagcattttggtccctgctccttgcccatagtaaaacagcttgaaatatcccatgcaagagagtagtttcaagtgggcgactctgctctctatttaaaagcgtgcacaatc

SEQ ID NO: 92 - DNA, 183 bp, Homo sapiens
attgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataa

SEQ ID NO: 93 - DNA, 254 bp, Homo sapiens
gaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaattctgttgattaaagatgagctttgtc

SEQ ID NO: 94 - DNA, 199 bp, Homo sapiens
agcagaagacgttttccctgagaagacatagaaagaaaatcaactttcactaaggcatttcagaaacataggctagggtaatatgtgtaccagtagagaagcctgaggaatttacaaaatgatgcagctccaagccattgtatggcccatgtgggagactgatgggacatggagaatgacagtagattatcaggaaata

SEQ ID NO: 95 - DNA, 237 bp, Homo sapiens
atgagtgcatttcaactatgtcaatggtttcttaatatttattgtgtagaagtactggtaatttttttatttacaatatgtttaaagagataacagtttgatatgttttcatgtgtttatagcagaagttatttatttttatggcattccagcggatattttggtgtttgcgaggcatgcagtcaatattttgtacagttagtggacagtattcagcaacgcctgatagcttctttg

SEQ ID NO: 96 - DNA, 188 bp, Homo sapiens
gtcactactgggcaaatacacttactgtgttctagaggcagccctttcttatgcagaaaatacaatacgcactgcatgagaagcttgagagtggattctaatccaggtctgtcgaccttggatatcatgcatgtgggaaggtgggtgtggtgagaaaagttttaaggcaagagtagatggccatgttc

SEQ ID NO: 97 - DNA, 48 bp, Homo sapiens
cttccttctctcttactcggagacagtcagaactctcctccctgacag

SEQ ID NO: 98 - DNA, 167 bp, Homo sapiens
tacccaattacccaggtcataaggtatgtctgtgtgacacttatctctgtgtatatcagcatacacacacacacacacacacacacacacacacacaggcatttccacacattacatatatacacatactggtaaaagaacaatcgtgtgcaggtggtcacacttcc

SEQ ID NO: 99 - DNA, 34 bp, Homo sapiens
gaaaaatatactaagttggtatactataacactt

SEQ ID NO: 100 - DNA, 277 bp, Homo sapiens
tgtccttccactcaacagtcatcaaccactaccgcatgcggggccatagcccctttgccaacctcaaatcgtgctgtgtgcccaccaagctgagacccatgtccatgttgtactatgatgatggtcaaaacatcatcaaaaaggacattcagaacatgatcgtggaggagtgtgggtgctcatagagttgcccagcccagggggaaagggagcaagagttgtccagagaagacagtggcaaaatgaagaaatttttaaggtttctgagttaaccaga

SEQ ID NO: 101 - DNA, 193 bp, Homo sapiens
aagggagaatggtgtaccctttatttcttctgaaatcacactgatgacatcagttgtttaaacggggtattgtcctttccccccttgaggttcccttgtgagcttgaatcaaccaatctgatctgcagtagtgtggactagaacaacccaaatagcatctagaaagccatgagtttgaaagggcccatcacag

SEQ ID NO: 102 - DNA, 125 bp, Homo sapiens
ttccttctctcttactcggagacagtcagaactctcctccctgacagccacaaacctacagcactgactgcattcagagaggaacctgcaaacaaaacttcacagaaaactttttgttcttgttc

SEQ ID NO: 103 - DNA, 264 bp, Homo sapiens
gcccagttcaccctgatttaggagaagccaggaatttcccaggaccctgaaggggccatgatggcaacagatttggaacctcagcctggccagacacaggccctccctgttccccagagaaaggggagcccactgtcctgggcctgcagaatttgggttttgcctgccagctgcactgatgctgcccctcatctttctgcccaacccttccctcaccttggcaccagacacccaggacttatttaaactctgttgcaagtgcaa

SEQ ID NO: 104 - DNA, 289 bp, Homo sapiens
tctgaactgaatgtgactggtctgtataaaatacatattagagttcaaagacttggtacattaaaaagaatgtaaaatatctcattagtagacttttttatattgattacatgttgaaatgataatattttggatatgcagggttaaatgaaatgttattaaagttaatttcacctgtttctttttaattttttgatgtggctactagaaaaccccaaattccatatgtggtttgcatttgtggctcccattatatttctgttggaaagctcagctctagaaggaaaga

SEQ ID NO: 105 - DNA, 61 bp, Homo sapiens
tagtctgaactgaatgtgactggtctgtataaaatacatattagagttcaaagacttggta

SEQ ID NO: 106 - DNA, 300 bp, Homo sapiens
agaaataatgtttcgggctgggcgcggtggctcacacctgtaatcccagcaatttgggaggctggggcgggcagatcacctgaggtcaggagtttgagactccagcctggccaacgtggtaaaaccctgtctttactaaaaatacaaaaattagctgggcatggtggcgggcacctgtaatcccagctgctcagggggctgaggcaggagaattgcttgaacctaggaggtggaggttgcagtgagccaagatcgcgccactgcacttcagcctggcctttagaacgagactccctccaa

SEQ ID NO: 107 - DNA, 288 bp, Homo sapiens
tcaaaagcagatggcctctggatcagtgccagagccattccccgggcagagaagcatctagaccttgtcactaccaagggtgcggctccacacccgcgggacccgagcctctagaaatgagggagaagacgactctgcctttctgcaatttagacgtcgcaaggatgaaaacgatgcggacaggctttccagcagtgagcctggggaaatctgcgtcacatgggcgacagaaggtccctcgaatccctcaatccccgtggcccgcaccactgtgtaataatctccaaa

SEQ ID NO: 108 - DNA, 35 bp, Homo sapiens
ggcgggcgcctatactcccagttgctcgggaggct

SEQ ID NO: 109 - DNA, 130 bp, Homo sapiens
gaaagagcatcaacagtaacggcaaacaggggagctaatgtcaccaacaaaacttgtgaggtcaacccaaagaaagaagggaataaatgagcaaaactttggaaagagtttctcacatgctgaaaacact

SEQ ID NO: 110 - DNA, 253 bp, Homo sapiens
tgtccaagccctgtgagactgaaaaagcactttgaggaaccttaaagaccttgtttgtacataagaactgctagcaaaagagacctcactcttctcttgctttcgtgagaaaggaggggcgtggatgtaggattgctgtggaaagcgaacacaaaacaacccagaatgactgattaagtgccttgcaaatctttattattatccaaacatttatgttcatactttcttgtgtacagatggtgctagtcaagat

SEQ ID NO: 111 - DNA, 37 bp, Homo sapiens
cactgcattcctgggcaacgacaacagtgaaactcca

SEQ ID NO: 112 - DNA, 263 bp, Homo sapiens
gtagtttccagctaatggatcacctggataagattgcccctgtctttgtgagagcagcaattagctctcaccctgagaaggcggcgtgttttgttgctttgcagccttggtgtagaggatggattgggtttgcgtcttttcatttgaaagtaggttatcgattagcacggatgccaatttactacagaggctgttgccaatgcctcttgatctcattatggtgtaaattttcaagattgtatcacattagctcaggagaattc

SEQ ID NO: 113 - DNA, 165 bp, Homo sapiens
ctcagggagcgaacgtggatgaaaaccacagggattccggacgccagaccccattttatacttcacttttctctacagtgttgttttgttgttgttggtttttattttttatactttggccataccacagagctagattgcccaggtctgggctgaataaaacaa

SEQ ID NO: 114 - DNA, 109 bp, Homo sapiens
aacatagcaaatggcatcactgtgtttgacttcttgtgaaatttctgtactttgtatataaaatacataaaacaatagattagaaatcaaaagatatctctggcctgca

SEQ ID NO: 115 - DNA, 191 bp, Homo sapiens
taggtggtagatattgaggccaagaatattgcaaaatacatgaagcttcatgcacttaaagaagtatttttagaataagaatttgcatacttacctagtgaaacttttctagaattatttttcactctaagtcatgtatgtttctctttgattatttgcatgttatgtttaataagctactagcaaaataa

SEQ ID NO: 116 - DNA, 277 bp, Homo sapiens
gtgccatctaccgaaactccacagtttccattgtgaatggcttctttggtgcagagttccaaaaattatgtagcccagctctttaattttgtaacatctaatgatatcaccgccttgaagtgattaaagtagattgcttaaagaattaaagctttaaagatgaaagatgttattgcttttgctggacatgaggaacagttgtaaagtttccaggtttacaataactttctggaaccctctcagtgaactgtttcttgtaaaagttttccctaagata

SEQ ID NO: 117 - DNA, 246 bp, Homo sapiens
aagcagtctactagattgtgatcccttgagatatggaaggatgcctttttttctctgcatttaaaaaaatcccccagcacttcccacagtgcctattgatacttggggagggtgcttggcacttattgaatatatgatcggccatcaagggaagaactattgtgctcagagacactgttgataaaaactcaggcaaagaaaatgaaatgcatatttgcaaagtgtattaggaagtgtttatgttgt

SEQ ID NO: 118 - DNA, 172 bp, Homo sapiens
agcccacccattgaagtctccttgggccaccaaaggtggtggccatggtaccggggacttgggagagtgagacccagtggagggagcaagaggagagggatgtcgggggggtggggcacggggtaggggaaatggggtgaacggtgctggcagttcggctagatttctgtct

SEQ ID NO: 119 - DNA, 157 bp, Homo sapiens
tatgaattccattcaaatcgttcctttttgttaacaaggggcatggggaggggtgggggtgggggggcagaggcgtctgaccccaggaacctgcagggcggggctgggtcggtgccctttaaggacaattttgaccttgttcaacctttccacaaag

SEQ ID NO: 120 - DNA, 36 bp, Homo sapiens
tgggggggcagaggcgtctgaccccaggaacctgca

SEQ ID NO: 121 - DNA, 251 bp, Homo sapiens
aacctcagggagagtaagctctagtccctctgtcctgtagaaagagccctgaagaatcagcaattttgttgctttattgtggcatctgttcgaggtttgcttcctctttaagtctgtttcttcattagcaatcatatcagttttaatgctactactaacaatgaacagtaacaataatatccccctcaattaatagagtgctttctatgtgcaaggcacttttcacgtgtcacctattttaacctttccaa

SEQ ID NO: 122 - DNA, 219 bp, Homo sapiens
tgttgctttattgtggcatctgttcgaggtttgcttcctctttaagtctgtttcttcattagcaatcatatcagttttaatgctactactaacaatgaacagtaacaataatatccccctcaattaatagagtgctttctatgtgcaaggcacttttcacgtgtcacctattttaacctttccaaccacataaataaaaaaggccattattagttgaat

SEQ ID NO: 123 - DNA, 267 bp, Homo sapiens
ttgctttgtatgcactttgtttttttctttgggtcttgttttttttttccacttagaaattgcatttcctgacagaaggactcaggttgtctgaagtcactgcacagtgcatctcagcccacatagtgatggttcccctgttcactctacttagcatgtccctaccgagtctcttctccactggatggaggaaaaccaagccgtggcttcccgctcagccctccctgcccctcccttcaaccattccccatgggaaatgtcaacaag

SEQ ID NO: 124 - DNA, 152 bp, Homo sapiens
caccatgggcggcaacgctgaaggacagccctgcaagtttccattccgcttccagggcacatcctatgacagctgcaccactgagggccgcacggatggctaccgctggtgcggcaccactgaggactacgaccgcgacaagaagtatggct

SEQ ID NO: 125 - DNA, 126 bp, Homo sapiens
ttcctctctgtaattggttatcaggaagaatttgcttaatgactaacaccctaagcatcagacctggaatttggagttgcaaagtgactatcttcccatttcccatctcattttcaataacttcag

SEQ ID NO: 126 - DNA, 199 bp, Homo sapiens
tctgcttagccctaggagtctggttccctgccttcagttgcagagtgatgtgtttgtgtcattgttgatgtcacctcctaaaaagaccttcactttctggctgccacaaagccatatgtgttgctccccatatacagcctgacagagtaaatggagaggaagtgctggatttgtgtatcactggctatcagttcctcat

SEQ ID NO: 127 - DNA, 224 bp, Homo sapiens
ccagtataattaggggtgtttcagagcatccccagttatttagcacaacactgaaggagcacatcccctctccattttgacttctctccccacttttacagccactgccttcgtcagttttgtagaggtttgatttccatgtgggttttgttgtcattgttttgcatttttgttttgttattgatattgtttgctttcattgctaaaactcatatacgacttac

SEQ ID NO: 128 - DNA, 230 bp, Homo sapiens
ttggtccatttcttacaagtcatgtatcccaaactgtgtcagcactggcaagtgatctggatgatggctgcagtgatgctggtcttgactgttgtgctggggctctacaattcctataactcttgtgcagagcaggctgatgggccccttggaagatccacttgctcggcagcccagaaggactcctggtggagctcaggactccagcatgagcagcctacagagcagta

SEQ ID NO: 129 - DNA, 249 bp, Homo sapiens
  misc_feature (150)..(151): n is a, c, g, or t
  misc_feature (153)..(154): n is a, c, g, or t
  misc_feature (156)..(160): n is a, c, g, or t
  misc_feature (162)..(167): n is a, c, g, or t
tggtgcaccttggacacgtggacaaggcccaggcgccctctgctcttctgccctcgatgccacatggcggtgaacacatctgaagccactatgtttcctggctctaaggctcgtctgtgtaacccataaaacctgctttgattccaaaannannannnnnannnnnnaaggaggtgccccgatgcttggggtgggtgttcctgccggggcttcaaggggtgacttctgtaggctgtccaggtccgaggc

SEQ ID NO: 130 - DNA, 89 bp, Homo sapiens
  misc_feature (60)..(60): n is a, c, g, or t
acagaatgagaccttccggcccaagcgtggcgctgcgggcactttggtagactgtgccancacggcgtgtgttgtgaaacgtgaaataa

SEQ ID NO: 131 - DNA, 91 bp, Homo sapiens
  misc_feature (28)..(28): n is a, c, g, or t
acaagcagtaccgtcagtacccacttgnctttagcccattatgggaatctctgatgcctttgtgaccactggagagtttaatcctcttggt

SEQ ID NO: 132 - DNA, 228 bp, Homo sapiens
ggaaaggtgatctgcactgtatcttgggtttgttggctatgcttcctttgatgacatatattatacagtatatatatacatatattttttttgttagagttctagccattttatttctccgcagggtcctttctcagacattactgcatgctgtatatggcgttagctgtgtgttgatcttctaaaagatgatagagtttactggtaattgtgtaatcagctcctgcc

SEQ ID NO: 133 - DNA, 183 bp, Homo sapiens
acagagtctacagacattaaaaggataataatggaatttttaaaaaataagagcactttgggaggccgaggtgggtggatcatttgaggccaggagttcaagaccagcctggccaacatggtgaaaccctgtctcgactaaaaatacaaaaaattagctgagcatggtggcagacacttgtaa

SEQ ID NO: 134 - DNA, 179 bp, Homo sapiens
agaggttataggtcactcctggggcctcttgggtcccccacgtgacagtgcctgggaatgtattattctgcagcatgacctgtgaccagcactgtctcagtttcactttcacatagatgtccctttcttggccagttatcccttccttttagcctagttcatccaatcctcactgggtg

SEQ ID NO: 135 - DNA, 274 bp, Homo sapiens
aaacagattcctgccagggagactcagggggacccctcgtctgttccctccaaggccgcatgactttgactggaattgtgagctggggccgtggatgtgccctgaaggacaagccaggcgtctacacgagagtctcacacttcttaccctggatccgcagtcacaccaaggaagagaatggcctggccctctgagggtccccagggaggaaacgggcaccacccgctttcttgctggttgtcatttttgcagtagagtcatctccatcagctgt

SEQ ID NO: 136 - DNA, 282 bp, Homo sapiens
ggatggcacacatcagctttcaggaaaagcacatgaaaggtgctagggctcttggagctgactgtaggtttgggagttgtctgtctccttgctctagattacagctctgtgtgttgtgtggggtctccatggtttgccaaattatctcttcctcacttagccacaaggctgacagttaggaactatctcttcttgattgcattaggttggctgcttcctgaatgcatatcaaaaggctccttcctttagttcagtgctttcaacctgagctgtgcatcagaa

SEQ ID NO: 137 - DNA, 203 bp, Homo sapiens
aaatcataaagccttcatcactcccacatttttcttacggtcggagcatcagaacaagcgtctagactccttgggaccgtgagttcctagagcttggctgggtttaggctgttctgtgcctccaaggactgtctggcaatgacttgtattggccaccaactgtagatgtatatatggtgcccttctgatgctaagactccaga

SEQ ID NO: 138 - DNA, 245 bp, Homo sapiens
tgggcttccctctacagtgtggaactcaggttgtttcttctttcctccctgaaatgacatgagtttgcagcggatggtgaactgaagaaaccataggaggctctgtcttcttgcctgaatttcagttggaagcttggagatttggggttcaacagagataaggaagtgtaagccttcatcccgtctggtggttggcgatcacacaccgctctgtgctgaggctaatggccatgatcagagttgac

SEQ ID NO: 139 - DNA, 92 bp, Homo sapiens
ctcaaaaaagtggttttgaccagagaggcccagatggaggctgttcattccctgcagtgtcggcattgtaaataaagcctgagcacttgctg

SEQ ID NO: 140 - DNA, 238 bp, Homo sapiens
gagcaggagcctctacacagcctctggctcttaggtcccagtcatgtttgcaccccctcaaaggggcaggaccagcccttcctttcagtgtccataccaggggccttccatgtgctgatgggtgatgtgactgtggtcagcaggcttgggaagtgctgctgctgtagcttgagttgggctggggtcttggtaggacgctgatctcagaagtccccaaagttcactgtgtaggtctcta

SEQ ID NO: 141 - DNA, 128 bp, Homo sapiens
gaccagacagtgctgttggtgactgtccctgacttcagtgccacgcagaccgcctgccttgtgaacctgcgcagcctggcctgccagcccatcagcttctcgggcttcggggcagaggacgatgacct

SEQ ID NO: 142 - DNA, 219 bp, Homo sapiens
  misc_feature (48)..(48): n is a, c, g, or t
attgctagctgttaaacaagcccaaatttgatatactttttatatttnaaaaattatattcactgcccccataagagcaatcaaggcatgtctttaaattctatacatagatatagccaaaaatagtgcatttagtaacattcttttccaaaactatattcttgggaatgaatatctgtttcttctaacagtttgagtgataatctatacctgtagata

SEQ ID NO: 143 - DNA, 245 bp, Homo sapiens
atgggtggctaactctgggaagatacttgtgttaaactttatatgacatttaataacccttcatcataaggcaatgttttttacaaaaagattgaaaaaatcatgtaagtcatttactctgcaaaaatggcacattaggtggggttccaaaatccataatgaaacaatgtgttttgcaactaagaaacattcattatgatatatggaaaacactgtctgtctacttgtcctttacgaaaaaatgt

SEQ ID NO: 144 - DNA, 227 bp, Homo sapiens
ttataaaaataggtgcttggtgtacaaaactttttatatcaaaaggctttgcacatttctatatgagtgggtttactggtaaattatgttattttttacaactaattttgtactctcagaatgtttgtcatatgcttcttgcaatgcatattttttaatctcaaacgtttcaataaaaccatttttcagatataaagagaattacttcaaattgagtaattcagaaa

SEQ ID NO: 145 - DNA, 62 bp, Homo sapiens
ggacttgggaacaggactttatacctcttttactgtaacaagtactcattaaaggaaattga

SEQ ID NO: 146 - DNA, 66 bp, Homo sapiens
gtttggacttgggaacaggactttataccttttttactgtaacaagtactcattaaaggaaattga

SEQ ID NO: 147 - DNA, 225 bp, Homo sapiens
  misc_feature (114)..(114): n is a, c, g, or t
  misc_feature (141)..(141): n is a, c, g, or t
  misc_feature (146)..(147): n is a, c, g, or t
caaattcaagtcactagacttcagagttcaacacctggacatgagaagatattatattatgtaccataaatatttcctgtatctgactgcctgaacatattaccacatcctgtnccttagtatcctacagagacaaaaaanaaaannaaaaaaaaaaaaaacctttgttcaggtttttatgtaaatatttttcatgttccactttctatactttcagcaactaaa

SEQ ID NO: 148 - DNA, 167 bp, Homo sapiens
atatgagtgggtttactggtaaattatgttattttttacaactaattttgtactttcagaatgtttgtcatatgcttcttgcaatgcatattttttaatttcaaacgtttcaataaaaccatttttcagatataaagagaattacttcaaattgagtaattcagaaa

SEQ ID NO: 149 - DNA, 256 bp, Homo sapiens
atgactgcaaaaatgggtggctaactctgggaagatacttgtgttaaactttatatgacatttaataacccttcatcataaggcaatgttttttacaaaaagattgaaaaaatcatgtaagtcatttactctgcaaaaatggcacattaggtggggttccaaaatccataatgaaacaatgtgttttgcaactaagaaacattcattatgatatatggaaaacactgtctgtctacttgtcctttacgaaaaaatg

SEQ ID NO: 150 - DNA, 266 bp, Homo sapiens
gcaagccagccgccggtgctgttgacccaagggccgtggtccacggtacttgaagaagccagagcccacatcctgtgcactgctgaaggaccctacgctcggtggcctggcacctcactttgagaagagtgagcacactggctttgcatcctggaagacctgcagggggcggggcaggaaatgtacctgaaaaggattttagaaaaccctgggaaaacccaccacaccaccacaaaatggcctttagtgtatgaaatgcacatgga

SEQ ID NO: 151 - DNA, 133 bp, Homo sapiens
gatgggccttcagggaagttcctggcaggaacagtccttccatttgtttgggcggatggtccggaagagcaaagccatgctaatgagacagaggaatagcaagtccccttcaagccgacaggaaggggcctca

SEQ ID NO: 152 - DNA, 287 bp, Homo sapiens
gattggattaaagacctggcacttcagtaactcagcacgcttccacttcactcaacttaagagagttcattgacagtgttaggatgtgaaggctgggaaacacttattttgcttcaagagttccacttggctctcccaaataggtacctcaaaaactgttagcaagcggcatttggatgtcttgacaggggctttgcagggatttttagggttttttccacattgtccacattaatggttggcatgattgtgcttgcaggccaagaaatgatcataccccttgccaa

SEQ ID NO: 153 - DNA, 243 bp, Homo sapiens
acatcattgttgaaaccagaccctgtagtccagtggtgctgccctgttgtgcaaactgctcctttttctcgtgtttttgtaaagagcttccatctgggctggacccagttcttgcacatacaagacaccgctgcagtcagctaggacctttccgccatgtattctattctgtagtaaagcatttccatcaacaatgcctaattgtatctgttatttttggtttaacacacactgattcatact

SEQ ID NO: 154 - DNA, 284 bp, Homo sapiens
ttcgcatttaaggtataggttgttttaggcatgggcagtaggcgctttcagcacaggctccctgctggtcactctttgctgtagtttcctgctccatccatggggtcggaggtcttacctcctgacgttagagtcatttgcagcaggtgggagtgaaagtgggccgggtgccaggtcttccttacctgaacctctcctgtcttcctggcacattaggaatgtccaagcttatttctattggtgaatgctcccacatgggatggataatggcctgtggaacattc

SEQ ID NO: 155 - DNA, 35 bp, Homo sapiens
tcacatcagaaaactaaattccggctgggtgcggt

SEQ ID NO: 156 - DNA, 128 bp, Homo sapiens
actgaagtccagaggggtttcttgaactgcccaaggacacactactatttagtgatacaagctgggactaaaactgaggtcccccgacacctggtccagagttctttctactatacatcaaagaatat

SEQ ID NO: 157 - DNA, 209 bp, Homo sapiens
gatcctatcagtttcccatggtgccggggggagaccggtctccttccagaatgcttccgccatgcaccaccacctcgaatggcagcacgctattaaatccaaatttgcctaaccagaatgatggtgttgacgctgatggaagccacagcagttccccaactgttttgaattctagtggcagaatggatgaatctgtttggcgaccatat

SEQ ID NO: 158 - DNA, 208 bp, Homo sapiens
gacagaagcttgatgactctaaacctagtttgttctctgaccgcctcagtgatttagggcgcattcctcatcccagtatgagagtaggtgtcccgcctcagaacccacggccctccctgaactctgcaccaagtccttgtaatccacaaggacagagtcagattacagaccccaggcaggcacagtcttccccgccgtggtcctatga

SEQ ID NO: 159 - DNA, 180 bp, Homo sapiens
ttttttccaattgctattgcccaagaattgctttccatgcacatattgtaaaaattccgctttgtgccacaggtcatgattgtggatgagtttactcttaacttcaaagggactatttgtattgtatgttgcaactgtaaattgaattatttggcatttttctcatgattgtaatattaa

SEQ ID NO: 160 - DNA, 127 bp, Homo sapiens
gggcccctaatatcccagtttaatattccaataccctagaagaaaacccgagggacagcagattccacaggacacgaaggctgcccctgtaaggtttcaatgcatacaataaaagagctttatccct

SEQ ID NO: 161 - DNA, 224 bp, Homo sapiens
tgttacttcgttcctcctcctattttgagctatgcgaaatatcatatgaagagaaacagctcttgaggaatttggtggtcctctacttctagcctggttttatctaaacactgcaggaagtcaccgttcataagaactcttagttacctgtgttggataaggcacggacagcttctctgctctgggggtatttctgtactaggatcagtgatcctcccgggagg

SEQ ID NO: 162 - DNA, 237 bp, Homo sapiens
tccagctacaatacttccatttattagaagcacattaaccatttctatagcatgatttcttcaagtaaaaggcaaaagatataaattttataattgacttgagtactttaagccttgtttaaaacatttcttacttaacttttgcaaattaaacccattgtagcttacctgtaatatacatagtagtttacctttaaaagttgtaaaaatattgctttaaccaacactgtaaatatt

SEQ ID NO: 163 - DNA, 172 bp, Homo sapiens
tgcagtgctagtcccggcatcctgatggctccgacaggcctgctccagagcacggctgaccatttttgctccgggatctcagctcccgttccccaagcacactcctagctgctccagtctcagcctgggcagcttccccctgccttttgcacgtttgcatccccagcatttc

SEQ ID NO: 164 - DNA, 227 bp, Homo sapiens
gttgtggggtcaaccgtacaatggtgtgggagtgacgatgatgtgaatatttagaatgtaccatattttttgtaaattatttatgtttttctaaacaaatttatcgtataggttgatgaaacgtcatgtgttttgccaaagactgtaaatatttatttatgtgttcacatggtcaaaatttcaccactgaaaccctgcacttagctagaacctcatttttaaagatt

SEQ ID NO: 165 - DNA, 116 bp, Homo sapiens
ttcctttgaggttgatcgttgtgttgttttgctgcactttttacttttttgcgtgtggagctgtattcccgagaccaacgaagcgttgggatacttcattaaatgtagcgactgtc

SEQ ID NO: 166 - DNA, 224 bp, Homo sapiens
gcggtgttcctaatcacatgtttgtactataatttcacgtgggttcagttaaactagagctaaggttgtccatggcatgttgaatcaaaaataaaaatggtcagtgagtaaaagtaacgtactatgacaaacgtgacttacacctgtatgtgagcaaacaccttgtgtgtgtgtgtgtgtgtgtgtgtgtgttaccagcatacaccctagtaggtgcagcctcc

SEQ ID NO: 167 - DNA, 51 bp, Homo sapiens
ttcacgtgggttcagttaaactagagctaaggttgtccatggcatgttgaa

SEQ ID NO: 168 - DNA, 144 bp, Homo sapiens
ggcacacccaataatcagtcatgtgtaatatgcacaagtttgtttttgtttttgttttttttgttggttggtttgtttttttgctttaagttgcatgatctttctgcaggaaatagtcactcatcccactccacataaggggtt

SEQ ID NO: 169 - DNA, 246 bp, Homo sapiens
accccaaacccaagtgccttcagaggataaatatcaatggaactcagagatgaacatctaacccactagaggaaaccagtttggtgatatatgagactttatgtggagtgaaaattgggcatgccattacattgctttttcttgtttgtttaaaaagaatgacgtttacatataaaatgtaattacttattgtatttatgtgtatatggagttgaagggaatactgtgcataagccattatgataa

SEQ ID NO: 170 - DNA, 217 bp, Homo sapiens
  misc_feature (29)..(29): n is a, c, g, or t
  misc_feature (68)..(68): n is a, c, g, or t
  misc_feature (177)..(177): n is a, c, g, or t
tgaaaacaagcatgagtagatcctgcttnccaatgggggccttcccgtctgcttactgcttaggtttnctgcggtgggtctgagcaatgcagaaagtttcttacaatttctctggattgaaacaagaaatagctccctcgctcctcttcataaatttctcagagaaaatctaagttnccaggaagtagccaaagaccaggagattgtggaatcaaaa

SEQ ID NO: 171 - DNA, 254 bp, Homo sapiens
tgagcttcccttggacactaactcttcccagatgatgacaatgaaattagtgcctgttttcttgcaaatttagcacttggaacatttaaagaaaggtctatgctgtcatatggggtttattgggaactatcctcctggccccaccctgccccttctttttggttttgacatcattcatttccacctgggaatttctggtgccatgccagaaagaatgaggaacctgtattcctcttcttcgtgataatataatc

SEQ ID NO: 172 - DNA, 225 bp, Homo sapiens
tagaggcttcttagattctcccagcatccgcctttccctttagccagtctgctgtcctgaaacccagaagtgatggagagaaaccaacaagagatctcgaaccctgtctagaaggaatgtatttgttgctaaatttcgtagcactgtttacagttttcctccatgttatttatgaattttatattccgtgaatgtatattgtcttgtaatgttgcataatgttca

SEQ ID NO: 173 - DNA, 257 bp, Homo sapiens
gaaagcctgagtttgcaaccagttgtagggtttttgttgtgttttttttttttttttgaaataaaactataatataaattctcctattaaataaaattattttaagttttagtgtcaaaagtgagatgctgagagtaggtgataatgtatattttacagagtgggggttggcaggatggtgacattgaacatgattgctctctgtctcttttttcagcttatgggtatttatcttctattagtatttgtatcttcag

SEQ ID NO: 174 - DNA, 267 bp, Homo sapiens
tcctgtaaaatacagtgcaggccaggcgcggtgactcacgcctgtaatcccagcattttgggaggccaagggtggcggatcatgaggtcaggagatcgagaccatcctggctaacacggtgaaaccccatctttactaaaaatacaaaaaattaaccaggtgtggtggcgcacatctgaagtcccagctacttgggaggctgaggcaggataatcacttaaacttgggaggtggaggttgcagtgagacgacacctcgccactgcac

SEQ ID NO: 175 - DNA, 169 bp, Homo sapiens
atccacagatatagccaagtagattgggtagaggatactatttccagaatagtgtttagctcacctagggggatatgttgtatacacatttgcatatacccacatgggggacataagctaatctttttacaggacacagaattctgttcaatgctgttaaatatgccaa

SEQ ID NO: 176 - DNA, 237 bp, Homo sapiens
tctgaacatcagtattgcagttgtggaagagcagaaggaggatacatttgtttgtgttgctccccaaaattccaccttgcatttgcatcacaaacttccctcaattgaggcagttttctttgttagaacattaagtctgtgtattgtaatagagtgggctcaatattttactataaagcatttaataaactgttattaatagaagtttgtgttcttcacacctttgctattgctttt

SEQ ID NO: 177 - DNA, 225 bp, Homo sapiens
tactgtccaatagcacctcattcccacattttattttcccccggtattctttagatcctagtatttggaaaacaatcggctaaccttgacatttctttttaccttcatatgccactatctcggtagttcaaaaaaatttagttcttgataaattgccttgaagtttaccttgtgctggagagccttatgataactccaaagactttcttacggtataatacatgt

SEQ ID NO: 178 - DNA, 280 bp, Homo sapiens
atttttctatttgtggtatatgtatatatgtacctatatgtatttgcatttgaaattttggaatcctgctctatgtacagttttgtattatactttttaaatcttgaactttataaacattttctgaaatcattgattattctacaaaaacatgattttaaacagctgtaaaatattctatgatatgaatgttttatgcattatttaagcctgtctctattgttggaatttcaggtcattttcataaatattgttgcaataaatatccttgaacacaaaa

SEQ ID NO: 179 - DNA, 204 bp, Homo sapiens
agaaggaatgactctagctacaataatacccagtatgtttaagcaggttcccttggttgttgcattaaaagtaatccccctttaggtattttagagcccagaacaaccctgtgttgatttagtaggtttttatttttcctttttttttacaatgcccataatactttcctgtatttatatcataacgtgtatagtgtaaaatgt

SEQ ID NO: 180 - DNA, 157 bp, Homo sapiens
ttacctgcatgaacccatctgcataccaaaggacttattctatgaaatactttaaaaattcctcatcagcaaaggacaattcaataaatacatccaaacatgatcatcgttggagccggaggtggcaggagtcgaggcgctgatccctaaaatggcg

SEQ ID NO: 181 - DNA, 237 bp, Homo sapiens
acaaaagctcccctgatccaactagcacactgtcaaatacagtgtcatatgagaggtccacagacggtagtttccaagaccgtttcagggaattcgaggattccaccttaaaacctaacagaaaaaaacccactgaaaatattatcatagacctggacaaagaggacaaggatttaatattgacaattacagagagtaccatccttgaaattctacctgagctgacatcggataaaa

SEQ ID NO: 182 - DNA, 115 bp, Homo sapiens
  misc_feature (81)..(81): n is a, c, g, or t
ttacctgcatgaaccgtaagtggtcctttagaaagaatggactaccgtgctataacaactactagacaccttcattttacnggctgtggtatcggcagtttagggtatggagcaa

SEQ ID NO: 183 - DNA, 230 bp, Homo sapiens
ttgctctttgactgtcagcaagactgaagatggcttttcctggacagctagaaaacacaaaatcttgtaggtcattgcacctatctcagccataggtgcagtttgcttctacatgatgctaaaggctgcgaatgggatcctgatggaactaaggactccaatgtcgaactcttctttgctgcattcctttttcttcacttacaagaaaggcctgaatggaggacttttct

SEQ ID NO: 184 - DNA, 272 bp, Homo sapiens
tgaataataaaatttaagtggccaggcatggtggcttacgcctgtaatcccagctctttgggaggtcaaggcgggcggatcaactgaggtcagaagttcgagaccagcctggccaacatggcgaaaccccatctttactaaaagtacaaaaattagccgggtgtggtggcgggtgcctgtaatttcagctaatcaggaggccgaggcaggagaatagcttgaacctgggaggcagaggttgcagtgagcagagatcatgccaccccactcca

SEQ ID NO: 185 - DNA, 113 bp, Homo sapiens
caaagtgctaataattaactcaaccaggtctactttttaatggctttcataacactaactcataaggttaccgatcaatgcatttcatacggatatagacctagggctctgga

SEQ ID NO: 186 - DNA, 238 bp, Homo sapiens
agttgtgctaccttaattttgtgtttccagaaataaaaattaaccttagttatgctgtcatttttaactaataaaaaaagtataattcataaaacttttggctttataagataattataaaattatatatttttttctgtttttgtggggttgggaaaacattttcttatttctattcactcttcaaatgcaggtctcataatatgtgtcaatgatataagatgatggaagactttgt

SEQ ID NO: 187 - DNA, 265 bp, Homo sapiens
aaaatacttctagttatggttgtattaattaatttgattgtgataatgattacacaatgtttacctatatcaaatcatcatatcgtataccttaaatatatataacttttatgtgtcaattataactcagtaagtctgggaaaatatcgttaagtcaaagattagagtcaacagaaataaagaaaaatcatactttgataacttcaggactaatcaaggatcaatgggtgacatgatatcatgctatgtgccattttgtgttaaa

SEQ ID NO: 188 - DNA, 224 bp, Homo sapiens; identical to SEQ ID NO: 17

SEQ ID NO: 189 - DNA, 273 bp, Homo sapiens; identical to SEQ ID NO: 22

SEQ ID NO: 190 - DNA, 260 bp, Homo sapiens; identical to SEQ ID NO: 21

SEQ ID NO: 191 - DNA, 256 bp, Homo sapiens; identical to SEQ ID NO: 20

SEQ ID NO: 192 - DNA, 129 bp, Homo sapiens; identical to SEQ ID NO: 25

SEQ ID NO: 193 - DNA, 267 bp, Homo sapiens; identical to SEQ ID NO: 18

SEQ ID NO: 194 - DNA, 257 bp, Homo sapiens; identical to SEQ ID NO: 19

SEQ ID NO: 195 - DNA, 240 bp, Homo sapiens; identical to SEQ ID NO: 23
  misc_feature (28)..(28): n is a, c, g, or t
  misc_feature (163)..(163): n is a, c, g, or t

SEQ ID NO: 196 - DNA, 221 bp, Homo sapiens; identical to SEQ ID NO: 24

SEQ ID NO: 197 - DNA, 287 bp, Homo sapiens; identical to SEQ ID NO: 152

SEQ ID NO: 198 - DNA, 266 bp, Homo sapiens; identical to SEQ ID NO: 150

SEQ ID NO: 199 - DNA, 133 bp, Homo sapiens; identical to SEQ ID NO: 151

SEQ ID NO: 200 - DNA, 243 bp, Homo sapiens; identical to SEQ ID NO: 153

SEQ ID NO: 201 - DNA, 284 bp, Homo sapiens; identical to SEQ ID NO: 154

SEQ ID NO: 202 - DNA, 255 bp, Homo sapiens
gatgccagcatgtgccaatgactgtcaccttcatctcttcaaaagaaaagccatagccgaggactgtcccgcgacccccgtggactgcgtttaggtcatgtgattctgttttcatttctcatcccatccaatttgtccttttctcctgtcattttcttcctctgtggtcccttcaaagttgttataatttgtactgaacttcaaaatgtgtcccgttctccccagaccactctagccacagtatattgcaataaa

SEQ ID NO: 203 - DNA, 238 bp, Homo sapiens
acccatcctgttcgtgaataggtctcaggggttgggggagggactgccagatttggacactatatttttttttaaattcaacttgaagatgtgtatttcccctgaccttcaaaaaatgttccaaggtaagcctcgtaaaggtcatcccaccatcaccaaagcctccgtttttaacaacctccaacacgatccatttagaggccaaatgtcattctgcaggtgccttcccgatggatta

SEQ ID NO: 204 - DNA, 239 bp, Homo sapiens
tctgttttaaggtctccttgaaggcgcactggggaccctggccatgcctcgttctccctgctttctttatcctgttattgcctccacagtctgttgccaaggactctaagatcaatgcacgtcactttcctttccactgggcaggatagccaagcacactccctcctgcgctctcccgccccggtgcgtccactcccgagggctgttatgaggactgggttgtgcctacttgatttgaa

SEQ ID NO: 205 - DNA, 192 bp, Homo sapiens
ggccaacgtggcaaaaccccatctttactaaaaatacaaaaattagctgggcatagtggtgtgcacctgtaattccagctactcgggaggctgaggcaggagaatctcttgaacccaggaggcagaggttgcagtgagctgagatcgcgccactgcactccatcctgggggacagggtgagactccgtctcc

SEQ ID NO: 206 - DNA, 178 bp, Homo sapiens
aaggacctaatataaccaaaacaattttgaaaaagaagaacatcggagggcttatgctttgtgattttgagacttgctttaaagctatagtgatgaagacagtgtagtgttgacagaaagacatatagaccaatggaatagaatcaagtccagaaatagactcacatgtaaataattt

SEQ ID NO: 207 - DNA, 185 bp, Homo sapiens
gggatgcaaaggacctaatataaccaaaacaattttgaaaaagaagaacatcggagggcttatgctttgtgattttgagacttgctttaaagctatagtgatgaagacagtgtagtgttgacagaaagacatatagaccaatggaatagaatcaagtccagaaatagactcacatgtaaataatt

SEQ ID NO: 208 - DNA, 272 bp, Homo sapiens
tccctccagcggcttctgttttttaggatgttaaggcgagtcccaggatggcattcaactggacacggatgccagtgttgtgagtgctccatgctgaaagccgccttttctggggcggttactcacgcagaggcgggaacctgactctgtgcctgtctgttggcttccgtaatccagccctccctgtgctgagtttaagcccaaatgaaattactgggctctaagtatgtgcctttctccataaaatgtgatgcctggtcaatatcttggcc

SEQ ID NO: 209 - DNA, 213 bp, Homo sapiens
cacatcacaaactgccctttcttggtcagaagaagcagagtggagccttctcatccccacgcgcgcagctgtggggccccgtggtcacctggccacatgggagtttgcatactgagtggttcatcttttccaatgtgttgtgtcctttaatttacatttatatttcattgccctttctaatgatcagaacagcacatcttgttatagaaaatt

SEQ ID NO: 210 - DNA, 251 bp, Homo sapiens
atataattggacaaacgctggcaaaaagaaaaaaatggtaagcaaaaaacccaagataaagtttcgaggacatcaggccttttgaaatacaatgtcaaatgacacattgtacggtttcaaaaaatccgctagacatgtcataagttttaactgtaatgcccaggaaaggatatcttaaaatattctaaacttgtgtaacaaaggaataattaactgtaatagtttttcaataaatcgagttgggtgtttcc

SEQ ID NO: 211 - DNA, 232 bp, Homo sapiens
atggaagagatcttcggctctctcaactctctgaagctggagattgagcagatgaaacggcccctgggcacgcagcagaaccccgcccgcacctgcaaggacctgcagctctgccaccccgacttcccagatggtgaatactgggtcgatcctaaacaatgatgctccagggattccttcaacgtttactgcaacttcacagccggggggtcgacatgcgtcttccctgaca

SEQ ID NO: 212 - DNA, 129 bp, Homo sapiens; identical to SEQ ID NO: 27

SEQ ID NO: 213 - DNA, 94 bp, Homo sapiens; identical to SEQ ID NO: 26

SEQ ID NO: 214 - DNA, 113 bp, Homo sapiens; identical to SEQ ID NO: 185

SEQ ID NO: 215 - DNA, 204 bp, Homo sapiens; identical to SEQ ID NO: 179

SEQ ID NO: 216 - DNA, 272 bp, Homo sapiens; identical to SEQ ID NO: 184

SEQ ID NO: 217 - DNA, 115 bp, Homo sapiens; identical to SEQ ID NO: 182
  misc_feature (81)..(81): n is a, c, g, or t

SEQ ID NO: 218 - DNA, 157 bp, Homo sapiens; identical to SEQ ID NO: 180

SEQ ID NO: 219 - DNA, 237 bp, Homo sapiens; identical to SEQ ID NO: 181

SEQ ID NO: 220 - DNA, 230 bp, Homo sapiens; identical to SEQ ID NO: 183

SEQ ID NO: 221 - DNA, 162 bp, Homo sapiens; identical to SEQ ID NO: 65

SEQ ID NO: 222 - DNA, 32 bp, Homo sapiens; identical to SEQ ID NO: 63

SEQ ID NO: 223 - DNA, 72 bp, Homo sapiens; identical to SEQ ID NO: 64

SEQ ID NO: 224 - DNA, 196 bp, Homo sapiens; identical to SEQ ID NO: 80

SEQ ID NO: 225 - DNA, 51 bp, Homo sapiens; identical to SEQ ID NO: 74

SEQ ID NO: 226 - DNA, 112 bp, Homo sapiens; identical to SEQ ID NO: 66

SEQ ID NO: 227 - DNA, 269 bp, Homo sapiens; identical to SEQ ID NO: 68

SEQ ID NO: 228 - DNA, 291 bp, Homo sapiens; identical to SEQ ID NO: 82

SEQ ID NO: 229 - DNA, 54 bp, Homo sapiens; identical to SEQ ID NO: 79

SEQ ID NO: 230 - DNA, 169 bp, Homo sapiens; identical to SEQ ID NO: 75

SEQ ID NO: 231 - DNA, 129 bp, Homo sapiens; identical to SEQ ID NO: 84

SEQ ID NO: 232 - DNA, 251 bp, Homo sapiens; identical to SEQ ID NO: 77

SEQ ID NO: 233 - DNA, 69 bp, Homo sapiens; identical to SEQ ID NO: 71

SEQ ID NO: 234 - DNA, 238 bp, Homo sapiens; identical to SEQ ID NO: 69

SEQ ID NO: 235 - DNA, 209 bp, Homo sapiens; identical to SEQ ID NO: 83

SEQ ID NO: 236 - DNA, 122 bp, Homo sapiens; identical to SEQ ID NO: 78

SEQ ID NO: 237 - DNA, 186 bp, Homo sapiens; identical to SEQ ID NO: 76

SEQ ID NO: 238 - DNA, 233 bp, Homo sapiens; identical to SEQ ID NO: 73

SEQ ID NO: 239 - DNA, 241 bp, Homo sapiens; identical to SEQ ID NO: 70

SEQ ID NO: 240 - DNA, 129 bp, Homo sapiens; identical to SEQ ID NO: 72

SEQ ID NO: 241 - DNA, 248 bp, Homo sapiens; identical to SEQ ID NO: 81

SEQ ID NO: 242 - DNA, 145 bp, Homo sapiens; identical to SEQ ID NO: 67

SEQ ID NO: 243 - DNA, 239 bp, Homo sapiens; identical to SEQ ID NO: 3

SEQ ID NO: 244 - DNA, 244 bp, Homo sapiens; identical to SEQ ID NO: 4

SEQ ID NO: 245 - DNA, 214 bp, Homo sapiens; identical to SEQ ID NO: 6

SEQ ID NO: 246 - DNA, 164 bp, Homo sapiens; identical to SEQ ID NO: 7

SEQ ID NO: 247 - DNA, 254 bp, Homo sapiens; identical to SEQ ID NO: 93

SEQ ID NO: 248 - DNA, 183 bp, Homo sapiens; identical to SEQ ID NO: 92

SEQ ID NO: 249 - DNA, 277 bp, Homo sapiens; identical to SEQ ID NO: 100

SEQ ID NO: 250 - DNA, 237 bp, Homo sapiens; identical to SEQ ID NO: 95

SEQ ID NO: 251 - DNA, 193 bp, Homo sapiens; identical to SEQ ID NO: 101

SEQ ID NO: 252 - DNA, 48 bp, Homo sapiens; identical to SEQ ID NO: 97

SEQ ID NO: 253 - DNA, 125 bp, Homo sapiens; identical to SEQ ID NO: 102

SEQ ID NO: 254 - DNA, 34 bp, Homo sapiens; identical to SEQ ID NO: 99

SEQ ID NO: 255 - DNA, 188 bp, Homo sapiens; identical to SEQ ID NO: 96

SEQ ID NO: 256 - DNA, 167 bp, Homo sapiens; identical to SEQ ID NO: 98

SEQ ID NO: 257 - DNA, 36 bp, Homo sapiens; identical to SEQ ID NO: 120

SEQ ID NO: 258 - DNA, 157 bp, Homo sapiens; identical to SEQ ID NO: 119

SEQ ID NO: 259 - DNA, 172 bp, Homo sapiens; identical to SEQ ID NO: 118

SEQ ID NO: 260 - DNA, 274 bp, Homo sapiens; identical to SEQ ID NO: 135

SEQ ID NO: 261 - DNA, 179 bp, Homo sapiens; identical to SEQ ID NO: 134

SEQ ID NO: 262 - DNA, 245 bp, Homo sapiens
gaaatacgaatgtagagatccctaatcatcaaattgttgattgaaagactgatcataaaccaatgctggtattgcaccttctggaactatgggcttgagaaaacccccaggatcacttctccttggcttccttcttttctgtgcttgcatcagtgtggactcctagaacgtgcgacctgcctcaagaaaatgcagttttcaaaaacagactcagcattcagcctccaatgaataagacatcttcc

SEQ ID NO: 263 - DNA, 271 bp, Homo sapiens
aggggcagccgtgcttatatttttatggttacaatggcacaaaattattatcaacctaactaaaacattccttttctcttttttcctgaattatcatggagttttttaattctctcttttggaatgtagattttttttaaatgctttacgatgtaaaatatttattttttacttattttggaagatctggctgaaggattattcatggaacaggaagaagcgtaaagactatccatgtcatctttgttgagagtcttcgtgactgtaagat

SEQ ID NO: 264 - DNA, 205 bp, Homo sapiens
tgcttttcatcctgtataactgcaactttgtgccggctcatattatgttctagagagctcatgtacaccatggtgaccatagttaataatactggattgtatgcttaaaatttgctaagagagtagatcttaagtcatcaccaaaaaaagtaactgtaagatgatggagatgtgttaattagcttgacggtggtaatcacaatat

SEQ ID NO: 265 - DNA, 270 bp, Homo sapiens
  misc_feature (114)..(117): n is a, c, g, or t
  misc_feature (119)..(119): n is a, c, g, or t
  misc_feature (121)..(125): n is a, c, g, or t
  misc_feature (128)..(130): n is a, c, g, or t
  misc_feature (133)..(139): n is a, c, g, or t
  misc_feature (141)..(144): n is a, c, g, or t
  misc_feature (186)..(186): n is a, c, g, or t
tgttccgcattcacttaacatgtgcgttaaaaggaacaaagctagctggaggtagcaaagttttgaacttgaatcagggattgctaagaaaacttacaactaatgaccacagannnnangnnnnntgnnntcnnnnnnntnnnnctagatgtgattgtagccgtggtgcctgggcagatggtaaanaaacatgctgtcctcttatgacaatatgtgcagagaaggccccaaacgctggggtgagaggaacaacaaactgactaactcacg

SEQ ID NO: 266 - DNA, 225 bp, Homo sapiens
aatgtttgcacactgaattgaagaattgttggttttttcttttttttgttttgtttttttttttttttttttttgcttttgacctcccatttttactatttgccaatacctttttctaggaatgtgcttttttttgtacacatttttatccattttacattctaaagcagtgtaagttgtatattactgtttcttatgtacaaggaacaacaataaatcatatgg

SEQ ID NO: 267 - DNA, 224 bp, Homo sapiens
ttcggccagggtgtcgaacatgccacggccaacaaacaggtgtgcaagccccgtaacccctgcacggatgggacccacgactgcaacaagaacgccaagtgcaactacctgggccactatagcgaccccatgtaccgctgcgagtgcaagcctggacgctggcaatggcatcatctgcggggaggacacagacctggatgctggcccaatgagaacctggtgtg

SEQ ID NO: 268 - DNA, 223 bp, Homo sapiens
gaacaaagagttggccaatgagctgaggcggcctcccctatgctatcacaacggagttcagtacagaaataacgaggaatggactgttgatagctgcactgagtgtcactgtcagaactcagttaccatctgcaaaaaggtgtcctgccccatcatgccctgctccaatgccacagttcctgatggagaatgctgtcctcgctgttggcccagcgactctgcg

SEQ ID NO: 269 - DNA, 144 bp, Homo sapiens; identical to SEQ ID NO: 168

SEQ ID NO: 270 - DNA, 116 bp, Homo sapiens; identical to SEQ ID NO: 165

SEQ ID NO: 271 - DNA, 246 bp, Homo sapiens; identical to SEQ ID NO: 169

SEQ ID NO: 272 - DNA, 51 bp, Homo sapiens; identical to SEQ ID NO: 167

SEQ ID NO: 273 - DNA, 224 bp, Homo sapiens; identical to SEQ ID NO: 166

SEQ ID NO: 274 - DNA, 227 bp, Homo sapiens; identical to SEQ ID NO: 164

SEQ ID NO: 275 - DNA, 296 bp, Homo sapiens; identical to SEQ ID NO: 90

SEQ ID NO: 276 - DNA, 240 bp, Homo sapiens; identical to SEQ ID NO: 89

SEQ ID NO: 277 - DNA, 238 bp, Homo sapiens; identical to SEQ ID NO: 91

SEQ ID NO: 278 - DNA, 18 bp, Homo sapiens
ctccatgtgccggatagc

SEQ ID NO: 279 - DNA, 22 bp, Homo sapiens
cgatttcaccagaagcctctac

SEQ ID NO: 280 - DNA, 20 bp, Homo sapiens
tgttgcagtgagggcaagaa

SEQ ID NO: 281 - DNA, 20 bp, Homo sapiens
gaccctggttgcttcaagga

SEQ ID NO: 282 - DNA, 19 bp, Homo sapiens
accactatgccgcgctctt

SEQ ID NO: 283 - DNA, 19 bp, Homo sapiens
ggtcgtagggctgctggaa

SEQ ID NO: 284 - DNA, 20 bp, Homo sapiens
tggtctaacggtttccccta

SEQ ID NO: 285 - DNA, 18 bp, Homo sapiens
gacctcggagcgagagtg

SEQ ID NO: 286 - DNA, 19 bp, Homo sapiens
agctacgccttctcggtct

SEQ ID NO: 287 - DNA, 23 bp, Homo sapiens
ccttctctggaaacaatgacatc

SEQ ID NO: 288 - DNA, 20 bp, Homo sapiens
aagtgggtccatgaacctaa

SEQ ID NO: 289 - DNA, 21 bp, Homo sapiens
aaattcactctgcccaggacg

SEQ ID NO: 290 - DNA, 24 bp, Homo sapiens
ccagaaagctcttgagtttattcc

SEQ ID NO: 291 - DNA, 22 bp, Homo sapiens
catctagttcccgaaccatctc

SEQ ID NO: 292 - DNA, 226 bp, Homo sapiens
taataatgcccttaatgtgagggtttgtaatggtgcttattaagaccaaagacttgttaaatgtatacaccaagtggtaatgaaatttctgtgactggcccacacgtgcatagaggtctgggaggaccaggaaacagcctcagtggccagaggatcaccagtgcatccttcatcacagcatgtgcaatatgccaagattaccctcggtcattcctgtcaacaaggg

SEQ ID NO: 293 - DNA, 276 bp, Homo sapiens
actgggcagcaaaagttgttccacagtggaaatttaggcatcctcaagtttcttcccagcttctgctgtgttttcttagagtaaattgccaatttctgtttttacaggaaatccttttttaaaaatggaatcagtgtggtccccatctactctgcaaaaattgcatttttctctattttcaaatgagatttgttcaagtttcaaaaccacgtgaaataataaatgtatagtagttttcttttccttgggcattgcttgatatgtgaaatgggttta

SEQ ID NO: 294 - DNA, 297 bp, Homo sapiens
gtgttgtgtacatgcaactgaccatttcactggcttcccatgccgctgttacagatgtttcagccggaaaaattttaattagacttgactaagggttgtcattttcctgtattgaaggacagtgggaatacatttcctttaagagaaatttgtctgcagttgtttaagttaaagcctccatggaaacaacaggatggactgtcagtgaccccttttatcttcttggaggagcctgttaaataaattcagacagtatatatagggagattttgtattaaattcttaccattctcgctg

SEQ ID NO: 295 - DNA, 227 bp, Homo sapiens
ggtagttgtcaatgagggtatcagtcaggtggttggaaaaagagaaatagttttctccgggaatcggaagcatgactcctccccgcaaccctccaccacacacatactcacacttttaattggcagtcaaagagtcaaatgacctgggatcaactaaggcagttctgggtctgtgattaagcttggtttcttgttgtgtaacttgctggctcagttatccaaactat

SEQ ID NO: 296 - DNA, 287 bp, Homo sapiens
atttcactttcaatagttgaagaacttggctttgtaaatctctcagaagcttgaaaatatcttgtctctaccccctcagcccatttcatttgccaataattattttgtaagtagggttgaaatgaactcagctggccttgtgaaatgtttaaacttgcacaaacaactacatttttgttcaacaaatagcagtttactcagccaaaatcactttggatattgccattacaaatactgttaaacttcagaaatcatgtctgtaaattagatgagccaaaataaaggac

SEQ ID NO: 297 - DNA, 297 bp, Homo sapiens
gactgtatccaattgctgtaacttttggtttcttaatgtcataatttttaaagtctgttttattttaagtgcaatattgagtatttagctgttaggctcaatccgtcgatatgaaataattttttaaatccctaagggcaggaaagcatttcgtggtagtgaaaataagaggaaataagatggcatgaaggtggtgggcggagaaactaggtagcacacaggaaagtgctctcaaaaatctttgaagagctcagctagaaaaaaatggagtagatttggctcatactattccggaag

SEQ ID NO: 298 - DNA, 266 bp, Homo sapiens
gtggctgtccacctggttacttccgcataggccaaaggcactgtgtttctggaatgggcatgggccgaggaaacccacagccacctgtcagtggtgaaatggatgacaaatcactctcccccacaggcttgttacgagtgtaagatcaatggctaccccccacggggcaggaaacggagaagcacaaacgaaactgatgcctcccatatcgaggatcagtctgagacagaaaccaatgtgagtcttgcaagttgggatgttgagaa

SEQ ID NO: 299 - DNA, 266 bp, Homo sapiens
cagtgcccactgtagccatggaggtgcggccatcaccaagcacccccctggaggtccctctcaatgcctccagcaccaatgccacagaggatcacccagaaaccaatgccgtagatgaaccctacaaaatccatggcctggaagatgaagtccagccacccaacctaatccctcgacgaccgagcccctatgaaaagaccagaaaccaaagaatccaagagctcattgatgagatgcggaagaaagagatgctcggaaagttctaa

SEQ ID NO: 300 - DNA, 276 bp, Homo sapiens
  misc_feature (42)..(43): n is a, c, g, or t
atctcacgactgttaacttccaactttgacgaggccatgcgnncgacacgcgcgactggggaaggtgcaatactgtcctgggcatatgcactttggtttatcagggatattttattagtgttttgtatgtatttaatgacgtattctataggttccattgtctttgcttttctggttttttttatgagacgttagcgtgtgccaaagtgactgtgaaaatgacctttcattcttactaatgatggaaagacctcctgtcatgcatcccacaaataa

SEQ ID NO: 301 - DNA, 38 bp, Homo sapiens
ggccccccttggacgggactatgtgtgcacctggcaag

SEQ ID NO: 302 - DNA, 147 bp, Homo sapiens
tatatacatggtgctcaatagcaacatcttagcagatgaagcagtttatgattccactccctcctgtatgacaggtagccactatactgaatcaaggtgctgaactcaaatcacaaaattctggcttaccgatacaacaaccaatac

SEQ ID NO: 303 - DNA, 163 bp, Homo sapiens
tgagcagactgcaccccaagctcccgactccaggtcccctgatcttggggcctgtttcccatgggattcaagagggacagccccagctttgtgtgtgtttaagcttaggaatcgcctttatggaaagggctatgtgggagagtcagctatcttgtctggtttt

SEQ ID NO: 304 - DNA, 156 bp, Homo sapiens
gcagcaacagcaaatcacgaccactgatagatgtttatttttgttggagacatgggatgattattttctgttctatttgtgcttagtccaattccttgcacatagtaggtacccaattcaattactattgaatgaattaagaattggttgccataa

SEQ ID NO: 305 - DNA, 105 bp, Homo sapiens
ggacctgaagggtgacatcccaggaggggcctctgaaatttcccacaccccagcgcctgtgctgaggactccctccatgtggccccaggtgccaccaataaaaat

SEQ ID NO: 306 - DNA, 45 bp, Homo sapiens
tgcttatacacttacactttatgcacaaaatgtagggttataata

SEQ ID NO: 307 - DNA, 296 bp, Homo sapiens
  misc_feature (75)..(75): n is a, c, g, or t
  misc_feature (262)..(262): n is a, c, g, or t
atgggtgtagatcaaggcaggagcaggaaccaaaaagaaaggcataaacataagaaaaaaaatggaaggggtggnaaacagagtacaataacatgagtaatttgatgggggctattatgaactgagaaatgaactttgaaaagtatcttggggccaaatcatgtagactcttgagtgatgtgttaaggaatgctatgagtgctgagagggcatcagaagtccttgagagcctccagagaaaggctcttaaaaatgcagcgcnatctccagtgacagaagatactgctagaaatctg

SEQ ID NO: 308 - DNA, 167 bp, Homo sapiens
tctgagcgggtcatggggcaacacggttagcggggagagcacggggtagccggagaagggcctctggagcaggtctggaggggccatggggcagtcctgggtgtggggacacagtcgggttgacccagggctgtctccctccagagcctccctccggacaatgagtc

SEQ ID NO: 309 - DNA, 60 bp, Homo sapiens
tttctggggtctttgagctccaaaaaataaacacttcctttgagggagagccccccccca

SEQ ID NO: 310 - DNA, 222 bp, Homo sapiens
gacacagtcgggttgacccagggctgtctccctccagagcctccctccggacaatgagtcccccctcttgtctcccaccctgagattgggcatggggtgcggtgtggggggcatgtgctgcctgttgttatgggttttttttgcggggggggttgcttttttctggggtctttgagctccaaaaaataaacacttcctttgagggagagcacaccttcccaa

SEQ ID NO: 311 - DNA, 264 bp, Homo sapiens
gccaacagcatcttttccgggttcctgctctttccagatatggaggcctgacctgtgggctgcttcacatccaccccggctccccctgccagcaacgctcactctacccccaacaccaccccttgcccagccaatgcacacagtagggcttggtgaatgctgctgagtgaatgagtaaataaacttttcaaggccaagggacagtggtttaattcaactctgtgtcccagcacctggcacaccagaagtgccatgctcagaaat

SEQ ID NO: 312 - DNA, 72 bp, Homo sapiens
gaatggcagtaccagaaggcattggttaagtgtcccaggaaccacacaagcagtgactcctaaagaagttca

SEQ ID NO: 313 - DNA, 239 bp, Homo sapiens
agatgcgctcgagacccacgggccttccacctccctcagcttcctgcatggacccaccttactggccagtctgcatccttgcctagaccattctcccctccagggagcccaccctgacccacccccactgcaccccctccccatgggttctctccttcctctgaacttctttaggagtcactgcttgtgtggttcctgggacacttaaccaatgccttctggtactgccattctttttt

SEQ ID NO: 314 - DNA, 284 bp, Homo sapiens
aactgaaatgtcaacctgtggactgtggcattcctgaatccattgagaatggtaaagttgaagacccagagagcactttgtttggttctgtcatccgctacacttgtgaggagccatattactacatggaaaatggaggaggtggggagtatcactgtgctggtaacgggagctgggtgaatgaggtgctgggcccggagctgccgaaatgtgttccagtctgtggagtccccagagaaccctttgaagaaaaacagaggataattggaggatccgatgcagat

SEQ ID NO: 315 - DNA, 201 bp, Homo sapiens
tggtgaatgcgtgctgcccaggaccagtgaagacagacatggatgggaaagacagcatcaggactgtggaggagggggctgagacccctgtctacttggccctcttgcctccagatgccactgagccacaaggccagttggtccatgacaaagttgtgcaaaactggtaaacgtctgcttcggagcttgctgcttaataaa

SEQ ID NO: 316 - DNA, 190 bp, Homo sapiens
cacagggaaatcagggttacaaatcttcttgatccacttctctcaggatcccctctcttcctacccttcctcaccacttccctcagtcccaactccttttccctatttccttctcctcctgtctttaaagcctgcctcttccaggaagacccccctattgctgctggggctccccatttgcttactttgc

SEQ ID NO: 317 - DNA, 115 bp, Homo sapiens
cagaaagccaagtggactcaacggagaggccagcaagtttcaggaaatggtgcatttggtgaacaaggagtcgtcagaaactccagaccagtttatgacagctgatgagacaagg

SEQ ID NO: 318 - DNA, 241 bp, Homo sapiens
ttcccatctcgtccatgagcctaggtcttggagccttgtgttggaggctgctgtgatgtcaggaacggggatctttctagcttttggccacttcctgggacctcacgcccctgttgacagatggagattgggcagcagggccttgctgcattgttatctgctgttccgacttggtttgtcttgtccaagggtgacgaaagagccaggcaccagggtctcatgggatgaggtccaacttttt

SEQ ID NO: 319 - DNA, 285 bp, Homo sapiens
caagacctaggctcatggacgagatgggaaggcacagggagaagggataaccctacacccagaccccaggctggacatgctgactgtcctctcccctccagcctttggccttggcttttctagcctatttacctgcaggctgagccactctcttccctttccccagcatcactccccaaggaagagccaatgttttccacccataatcctttctgccgacccctagttccctctgctcagccaagcttgttatcagctttcagggccatggttcacattagaata

SEQ ID NO: 320 - DNA, 37 bp, Homo sapiens
gatgcttaacaaaggttaccataagccacaaattcat

SEQ ID NO: 321 - DNA, 189 bp, Homo sapiens
ccctgcagagaatcacgtcctggaactgcatgttcttgcgactcttgggacttcattttaacttctcgctgccccagccatgttttcaaccatggcatccctcccccaattagttccctgtcatcctcgtcaaccttctctgtaagtgcctggtaagcttgcccttgcttaagaactcaaaacatagct

SEQ ID NO: 322 - DNA, 82 bp, Homo sapiens
tttaaaatactcagaggacaggatcactgtggaatcgaatcagaagtggtggctggaattccacgcaccgatcagtactggg

SEQ ID NO: 323 - DNA, 34 bp, Homo sapiens
gaaatcattgtgggattgctagctttccctctta

SEQ ID NO: 324 - DNA, 82 bp, Homo sapiens
cagatgttggtatctgcagggatcctggaaccaaacccctgcagatactaagggctgacgatctaggtaagactggatttaa

SEQ ID NO: 325 - DNA, 107 bp, Homo sapiens
tactcccgaggctctgtacattgctgccacatactcctgccagcttgggggagtgttccttcaccctcacagtatttattatcctgcaccacctcactgttccccat

SEQ ID NO: 326 - DNA, 83 bp, Homo sapiens
ttccaccatcaaatgctgtagaatgcttggcactccctaaccaaatgctgtctccataatgccactggtgttaagatatattt

SEQ ID NO: 327 - DNA, 88 bp, Homo sapiens
gccctcatggttggcatcacatatgcctgcatgccattaacaccagctggccctacccctataatgatcctgtgtcctaaattaatat

SEQ ID NO: 328 - DNA, 33 bp, Homo sapiens
ttctgtgcctcagccgttcttgacatcaagaat

SEQ ID NO: 329 - DNA, 241 bp, Homo sapiens
aactggaccctgtcgttctccatcttctcagtcagttgtttcaagtgttcctgataactcctctccttctgttccatcatctgctcattctttctttgcatttcctgcaacatttttgctgaagcctgtgcagactcagctttcacaagttccacttcaatctccttttctttttctgtgagagtctggtctgtctggagaattgcatcagtcatagactccttggatttcaagtatgtct

SEQ ID NO: 330 - DNA, 251 bp, Homo sapiens
gaacaggagcaactactaaaagagggatttcaaaaagaaagcagaataatgaaaaatgagatacaggatctccagacgaaaatgagacgacgaaaggcatgtaccataagctaaagaccagagccttcctgtcacccctaaccaaggcataattgaaacaattttagaatttggaacaagcgtcactacatttgataataattagatcttgcatcataacaccaaaagtttataaaggcatgtggtacaat

SEQ ID NO: 331 - DNA, 220 bp, Homo sapiens
gaacaggaacgccttctcaaggagggattcgagaatgagagcaagagacttcaaaaagacatatgggatatccagatgagaagcaaatcattggagccaatatgtaacatactttaaaagtccaaggagcaaaatttgcctgtccagctccctctccccaagaaacaacatgaatgagcaacttcagagtgtcaaacaactgccattaaacttaactcaa

SEQ ID NO: 332 - DNA, 82 bp, Homo sapiens
gttatttaagatggctatccagataatcctgaacactgtgtatttattttatttagactaccagcaaagattaaagcatgaa

SEQ ID NO: 333 - DNA, 201 bp, Homo sapiens
ccctgctgcctctgatcgtaggaattgaggagtgtcccgccttgtggctgagaactggacagtggcaggggctggagatgggtgtgtgtgtgtgtgtgtgtgtgtgtgtgtgtgtgcgcgcgcgccagtgcaagaccgagattgagggaaagcatgtctgctgggtgtgaccatgtttcctctcaataaagttcccctgtg

SEQ ID NO: 334 - DNA, 262 bp, Homo sapiens
gtcctctctatcctggatgagctcatgaacatttctcttgtgttcctgactccttcccaatgaacacctttctgccaccccaagctttgctctcctcctctgtgagctctgggcttcccagtttgtttacccgggaaagtacgtctagattgtgtggtttgcctcattgtgctatttgcccactttccttccctgaagaaatatctgtgaaccttctttctgttcagtcctaaaattcgaaataaagtgagactatggttca

SEQ ID NO: 335 - DNA, 135 bp, Homo sapiens
tacccgggaaagtacgtctagattgtgtggtttgcctcattgtgctatttgcccactttccttccctgaagaaatatctgtgaaccttctttctgttcagtcctaaaattcgaaataaagtgagactatggttca

SEQ ID NO: 336 - DNA, 278 bp, Homo sapiens
tgagaggcaagagttgttcctgcccttccctttgtgacttgaagaaccctgactttgtttctgcaaaggcacctgcatgtgtctgtgttcgtgtaggcataatgtgaggaggtggggagagcaccccacccccatgtccaccatgaccctcttcccacgctgacctgtgctccctctccaatcatctttcctgttccagagaggtggggctgaggtgtctccatctctgtctcaacttcatggtgcactgagctgtaacttcttccttccctattaaa

SEQ ID NO: 337 - DNA, 63 bp, Homo sapiens
attagaaccttagtataaatttactttctcaaattcttgccatgagaggttgatgagttaatt

SEQ ID NO: 338 - DNA, 75 bp, Homo sapiens
agaacctgagtataaatttactttctcaaattcttgccatgagaggttgatgagttaattaaaggagaagattcc

SEQ ID NO: 339 - DNA, 132 bp, Homo sapiens
acagcccacccttgtgtccactgtgacccctgttcccatgctgacctgtgtttcctccccagtcatctttcttgttccagagaggtggggctggatgtctccatctctgtctcaactttacgtgcactgagc

SEQ ID NO: 340 - DNA, 132 bp, Homo sapiens
acagcccacccttgtgtccactgtgacccctgttcccatgctgacctgtgtttcctccccagtcatctttcttgttccagagaggtggggctggatgtctccatctctgtctcaactttatgtgcactgagc

SEQ ID NO: 341 - DNA, 273 bp, Homo sapiens
ttcaaactcaatgatgctaccatgcctctccaacattttcaaccccctgacattatcttggatcctatggtttctccatccaattctttgaatttcccagtctcccctatgtaaaacttagcaacttgggggacctcattcctgggactatgctgtaaccaaattattgtccaaggctatatttttgggatgaatataatttgaggaagggagttaaagaccctcctggggctctcagtgtgccatagaggacagcaactggtgattgtttca

SEQ ID NO: 342 - DNA, 89 bp, Homo sapiens
tgactgaattgctgacccttcaagctctgtccttatccattacctcaaagcagtcattccttagtaaagtttccaacaaatagaaatta

SEQ ID NO: 343 - DNA, 279 bp, Homo sapiens
gcatttgctgtgtttcgttagcatctggctccaggacagaccttcaacttccaaattggatactgctgccaagaagttgctctgaagtcagtttctatcattttgctctttgattcaaagcactgtttctctcactgggcctccaaccatgttcccttttttttagcaccacaaataatcaaaacccaacatgactgtttgttttcctttaaaaatatgcaccaaatcatctctcatcacttttctttgagggttttagtagacagtaggagttaataa

SEQ ID NO: 344 - DNA, 53 bp, Homo sapiens
aagcaagatatcaatgtagcagaattgcacttgtgcctcacgaacatacataa

SEQ ID NO: 345 - DNA, 159 bp, Homo sapiens
gagcttccttcttcgttcttggcaccatcttatgaaaagggtccagattaagatttttgactgagtcattctaaagtaagttgcaagacccatgatactagaccactaaatacttcatcacacacctcctaagaataagaaccaacattatcacaccaa

SEQ ID NO: 346 - DNA, 159 bp, Homo sapiens; identical to SEQ ID NO: 345

SEQ ID NO: 347 - DNA, 232 bp, Homo sapiens
tgaaccaaaatagagtcagctgacccagcatcagccacactttgggttggaaaatgtttgcctgttggaattaatttaagcttaagtatatatcaacattattttattgtgcaattaaaacaatacaaattcatggttttttaaagttaaaaattttaaccactgtaacaacagtttttgtgttattttctgtattaaacatcttgttgcacgcatttgaggtcatcagggt

SEQ ID NO: 348 - DNA, 186 bp, Homo sapiens
gagacttgtatgaaagatggctgtgcctctgcctgtctcccccaccgggctgggagctctgcagagcaggaaacatgactcgtatatgtctcaggtccctgcagggccaagcacctagcctcgctcttggcaggtactcagcgaatgaatgctgtatatgttgggtgcaaagttccctacttcctg

SEQ ID NO: 349 - DNA, 243 bp, Homo sapiens
aaaacttggcactttttcgtgtggatcttgccacatttctgatcagaggtgtacactaacatttcccccgagctcttggcctttgcatttatttatacagtgccttgctcggcgcccaccaccccctcaagccccagcagccctcaacaggcccagggagggaagtgtgagcgccttggtatgacttaaaattggaaatgtcatctaaccattaagtcatgtgtgaacacataaggacgtgtg

SEQ ID NO: 350 - DNA, 174 bp, Homo sapiens
ttgcatggagacttgaggagggagggcttgaggttggtgaggttaggtgcgtgtttcctgtgcaagtcaggacatcagtctgattaaaggtggtgccaatttatttacatttaaacttgtcagggtataaaatgacatcccattaattatattgttaatcaatcacgtgtatag

SEQ ID NO: 351 - DNA, 296 bp, Homo sapiens
tgaggatgtcaccaattaaccagaaatccagttattttccgccctcaaaatgacagccatggccggccgggtgcttttgggggctcgtcggggggacagctccactttgactggcacagtctttgcatggagacttgaggagggagggcttgaggttggtgaggttaggtgcgtgtttcctgtgcaagtcaggacatcagtttgattaaaggtggtgccaatttatttacatttaaacttgtcagggtataaaatgacatcccattaattatattgttaatcaatcacgtgtatag

SEQ ID NO: 352 - DNA, 227 bp, Homo sapiens
gaagatgaagccccatgctcagtcccctcccatcccccacgcagctccaccccagtcccaagccaccagctgtttgctcctggtgggaggtggcctcctcagcccctcctttctgacctttaacctcactctcaccttgcaccgtgcaccaacccttcacccctcctggaaagcaggcctgatggcttcccactggcctccaccacctgaccagagtgttctcttca

SEQ ID NO: 353 - DNA, 38 bp, Homo sapiens
agtctgcctatgatctttgaatgagctttttaaggaag

SEQ ID NO: 354 - DNA, 174 bp, Homo sapiens
tgggcgtccgggcccccaatattcacgcactcgcaccacgcactcatattccctcaccccaccatcacggccccaaagaaggtcttccctctcgcgaagtccaccatatcggggtgactgatgttggacgtacaccctctcgcccctccggagctgcaccaggccgccgaaccc

SEQ ID NO: 355 - DNA, 292 bp, Homo sapiens
acagtgttgtatgaggtttgaggattttgatccaagctggtcccactcagtccatagcagagaatgaaagggcccagagagggtggtgacctctgcctgaagtcacacagtgagtcgaggacagggaggtgaccccaggtttctatgtgtagggcgggaggatgttttgggacacagttcaattctcatttgtcacacactttggctattagagatcaaccccttcgctcctgtgtcttgcaatggcagccttggcaaacgctaaatgaaaatcgtgacaacacttgtgtta

SEQ ID NO: 356 - DNA, 151 bp, Homo sapiens
tagggattaacttcctgtatgctgcaactcatgaacttggccattctttgggtatgggacattcctctgatcctaatgcagtgatgtatccaacctatggaaatggagatccccaaaattttaaactttcccaggatgatattaaaggcat

SEQ ID NO: 357 - DNA, 82 bp, Homo sapiens
gtgctcagtagtcagactggatagtccgtttttgcttatccgttagccgtggggatttagcaggaagctgtgagagcagttt

SEQ ID NO: 358 - DNA, 116 bp, Homo sapiens
tgctgtgaaagaggctggctacacaatcgaatggtttgaggtgatctcgcaaagttattcttccaccatggccaacaacgaaggacttttctccctggtggcgaggaagctgagca

SEQ ID NO: 359 - DNA, 228 bp, Homo sapiens
atgtaggagaacgtgccctattcacactttgggaagacgctaatttgtgacatttttttttcaagcctgccatcaaggacattttttaagacccaactggcatgagttggggtaatttcctattattttcattttggacaacttttttaacttatattctttatagaggattccccaaaatgtgctcctcatttttggcctctcatgttccaaacctcattgaataaa

SEQ ID NO: 360 - DNA, 37 bp, Homo sapiens
cagggactggctatcccaagacctggcagatgtggct

SEQ ID NO: 361 - DNA, 119 bp, Homo sapiens
gccagagagaccaagtgttatgtaagaagtagtgtcggctgtgtagaaccactgactacacaggccgaagttactgagaacttggacagaaaaaatagccagcaagtgttcaaactact

SEQ ID NO: 362 - DNA, 242 bp, Homo sapiens
catcttctagcagatctttttcagttaagattacttgttcttccatgtattcatatttagccagctccttgatcagccgcagtatgtcactgcagtcggcggcagtggctgggcggatcacgaatttagccattttcgtcttttgcttttcttccctttgcggaccaggcccctgtacttgaacagtaggaggaggtggttcctcattcgtctcccgggagcgtcctcttctcagtcaggct

SEQ ID NO: 363 - DNA, 180 bp, Homo sapiens
gcttccttaaaggtcagaacatcaggccaaagtacaacgtttaatttcagaacttgccttccaatttacgcattttcaatttgctctccccatttgttgagtcagaagaagcagcattgcccagaaacaggtattacgtaacatgcacatactttaaaaagtactcatcccttgttttct

SEQ ID NO: 364 - DNA, 272 bp, Homo sapiens
agagctcttgagtgggctagtgactccccctgcagcctggtggagatggtgtgaggagcgaagagccctctgctctaggatttgggttgaaaaacagagagagaagtggggagttgccacaggagctaacacgctgggaggcagttgggggcgggtgaactttgtgtagccgaggccgcaccctccctcattccaggctcattcattttcatgctccattgccagactcttgctgggagcccgtccagaatgtcctcccaataaaactccat

SEQ ID NO: 365 - DNA, 151 bp, Homo sapiens
acacagaagagtgacatgtttacaaacctcaagccagccttgctcctggctggggcctgttgaagatgcttgtattttacttttccattgtaattgctatcgccatcacagctgaacttgttgagatccccgtgttactgcctatcagcat

SEQ ID NO: 366 - DNA, 96 bp, Homo sapiens
tcgacacagtgctttccgtggcactgcatacaatctgaggcctcctctctcagtttttatatagatggcgagaacctaagtttcagttgattttac

SEQ ID NO: 367 - DNA, 107 bp, Homo sapiens
taaatataatatcgacacagtgctttccgtggcactgcatacaatctgaggcctcctctctcagtttttatatagatggcgagaacctaagtttcagttgattttac

SEQ ID NO: 368 - DNA, 252 bp, Homo sapiens
  misc_feature (180)..(182): n is a, c, g, or t
gtggagcagttggactgctctctctgctctcaggatgatactgtgagaacaatttaaatatgctaagcacatgtcaggaaacagttttgtggtctttggacactcgctgtagccattccgttccatttcaggtgattttattcatttcatttgtagaataaaataaatccatttcacacnnncacacacacacacacacacacacacacacaccctctatacaccactaaagcctcccattaaacccataga

SEQ ID NO: 369 - DNA, 121 bp, Homo sapiens
ggcagcgacccggaaacagcgtatcactgagaccgagtcgccttatcaggagctccagggtcagaggtcggatgtctacagcgacctcaacacacagaggccgtattacaaatgagcccga

SEQ ID NO: 370 - DNA, 123 bp, Homo sapiens
atggtataatggttgactgggtttctctgtatagtactggcatggtacggagatgtttcacgaagtttgttcatcagactcctgtgcaactttcccaatgtggcctaaaaatgcaacttcttt

SEQ ID NO: 371 - DNA, 273 bp, Homo sapiens
tagaagatgacctcgttccggagctgggtatttcaaatttgctttcatccagccactgcccaaagccattttcctgcctactggatgcttacagtgactgtggatacgggggttccctttccccattcagtgacatgtcctctttgcttggtgtaaaccattcttgggaggacacttttgccaatgaactctttccccagctgattagtgtctaaggaatgatccaatactgttgcccttttccttgactattacactgcctggaggatagca

SEQ ID NO: 372 - DNA, 231 bp, Homo sapiens
agctttgtgctcagatcccaggtcccaaggagtgacaggggcttcctcccaccttctgtccttgtccagtcatgtaaataatgtgctttttctctccccgagtcttttttttttaaacctaccgtggttcctcagctaactgcattccctacccaggcagagactgtcctatgcctcgagcttccaaacgagactcagaccgcgacacagccaccgtatttatggaatgac

SEQ ID NO: 373 - DNA, 182 bp, Homo sapiens
tgagtaggtgagtttattgggacttacacacaggtcaatcctgggcggcgacaagacagctctagagatctgagcttcctcccaatgctaaactgctttcatgctaattttctgactgtttacttaccgggtaagagcgatgggactgttttcattggttggttctcacatactctctggga

SEQ ID NO: 374 - DNA, 243 bp, Homo sapiens
tcccctgcttgaacactgaagggcaggtggtgggccatggccatggtccccagctgaggagcaggtgtccctgagaacccaaacttcccagagagtatgtgagaaccaaccaatgaaaacagtcccatcgctcttacccggtaagtaaacagtcagaaaattagcatgaaagcagtttagcattgggaggaagctcagatctctagagctgtcttgtcgccgcccaggattgacctgtgtgta

SEQ ID NO: 375 - DNA, 281 bp, Homo sapiens
ggagacagatggagggtaccctatttacaactgagtcagccaagccactgatgggaatatacagatttaggtgctaaaccatttattttccacggatgagtcacaatttgaagaatcaaacttccatcctgaaaatttatatgtttcaaaaccacttgccatcctgttagattgccagttcctgggaccaggcctcagactgtgaagtatatatcctccagcattcagtccagggggagccacggaaaccatgtttttgcttaagccattaaagtcagaga

SEQ ID NO: 376 - DNA, 196 bp, Homo sapiens
gcctttaagataccttgatgaagacctggactattgaatggagcagaaattcacctctctcactgactattacagttgcatttttatggagttcttcttctcctaggattcctaagactgctgctgaatttataaaaattaagtttgtgaatgtgactacttagtggtgtatatgagactttcaagggaattaaat

SEQ ID NO: 377 - DNA, 262 bp, Homo sapiens
gttcacccggtgaactatttatgagttcttttggtgtgaagaaagggctcatgttgcatttccagccattgctacaaagaacctttatttgttcagtaacggtagaaaatccttcccgattaaaaacttcagacttgctgaatatcctgcaatgtcaagatgaccgatgttgagttgggtggatttgctaacgagtcagatttgaacatgaggctattggaacccaataggcgtcattgatggcggcaagccatagctttca

SEQ ID NO: 378 - DNA, 155 bp, Homo sapiens
gttgttgatcatggatcatactccccttgtttctttgggtgagaagggatcgcagtttggaaactccggcggctgcgtgcggggtttcagtcccagctgtaggcttgtaaatacccgccccgccaaaccgcatagagaacgtggcagcaagctga

SEQ ID NO: 379 - DNA, 217 bp, Homo sapiens
atgctgtggttggatcaaggactcattcctgccttggagaaaatacttcaaccagagcagggagcctgggggtgtcggggcaggaggctggggatgggggtgggatatgagggtggcatgcagctgagggcagggccagggctggtgtccctaaggttgtacagactcttgtgaatatttgtattttccagatggaataaaaaggcccgtgtaatta

SEQ ID NO: 380 - DNA, 182 bp, Homo sapiens
atgctgagtgacactcttgtgtatatttccaaatttttgtacagtcgctgcacatatttgaaatcatatattaagactttccaaagatgaggtccctggtttttcatggcaacttgatcagtaaggatttcacctctgtttgtaactaaaaccatttactatatgttagacatgacattctt

SEQ ID NO: 381 - DNA, 259 bp, Homo sapiens
ctacaacaggcaggtactgctgccagggggctttgaactagtgcctgctacccaggacacccgggccatgcccctggctgggcagcctggcacaagtgaagaagaaggcagtgggaaaactgggtttatttcaaggcagcagcctgagcccaggagcagaggacccagttgttataaggcgctgggagaggatgggcagctcccactgccccagagcggagctcgaagcacccaggttgcccacggaaaatccaataaa

SEQ ID NO: 382 - DNA, 158 bp, Homo sapiens
tttggtgcagtttccagggtgcagtacagcagggcctgaatactggccctggactcccttttccagaacaccaggtgtggccacctggggctcaggtacacagtggggtctctcggaagccaccgtgtggttctttcacaggcacgtttattttgctg

SEQ ID NO: 383 - DNA, 267 bp, Homo sapiens
gcatttcaaagtgacttctatgaagctttttttttaatgtgaaattttcagaatgttgtttttttcatgtagatactccaggaagagttaagcactgctttcagttttaatatccaccttgaggggtcgctgcttgagggctcttatcccaggggactttttaattcggatgttacttaatgtggcttctctaatgtagtttctttgattaccgactacacaattatgtaccatcacagtattagtggaaaagtaccatgtgattta

SEQ ID NO: 384 - DNA, 94 bp, Homo sapiens
ggggagccaggcttccctcacgcagcctgtggtggatgtgggaaggagatcaacttctcctcactctgggacagacgatgtatggaaactaaaa

SEQ ID NO: 385 - DNA, 214 bp, Homo sapiens
gacttattcccgctgactgagtttttgaggggctaccaggaaagcgcctccaaccctagcaaaagtgcaagatggggagtgagaggctgggaatggaggggcagagccaggaagatcccccagaaaagaaagctacagaagaaactggggctcctccagggtggcagcaacaataaatagacacgcacggcagccacagcttgggtgtgtgttc

SEQ ID NO: 386 - DNA, 270 bp, Homo sapiens
ccagggctgagagacacgtgaaggaagatgatgggaggaaaagcccaggagaagtcccaccagggaccagcccagcctgcatacttgccacttggccaccaggactccttgttctgctctggcaagagactactctgcctgaacactgcttctcctggaccctggaagcagggactggttgagggagtggggaggtggtaagaacacctgacaacttctgaatattggacattttaaacacttacaaataaatccaagactgtcatattt

SEQ ID NO: 387 - DNA, 118 bp, Homo sapiens
tttgaagattagaaatttagccgtaggtaaagaatacaaaggaaaaataattttaaaatcatcaaccagatcaacaaaatatatgttaatgccgagactttgaattagagtgcgaatt

SEQ ID NO: 388 - DNA, 278 bp, Homo sapiens
ggaagccattatcctaaatgaactcactcagaaacagaaaaccaaataccacatgttctcacttataagtagaagctaaacattgagtacacatggatacaaagaagggaaccgcagacactggggcctacctgaggtcggagcatggaaggagggtgaggatcaaaaaactacctatctggtactatgctttttatctggatgatgaaataatctgtacaacaaaccctggtgacatgcaatttacctatatagcaagcctacacatgtgcccctga

SEQ ID NO: 389 - DNA, 173 bp, Homo sapiens
tattttccctctttcgaacaaagacattggtttgcccaaggactacaaataaaccaacgggaaaaaagaaaggttccagttttgtctgaaaattctgattaagcctctgggccctacagcctggagaacctggagaatcctacacccacagaacccggctttgtccccaaaga

SEQ ID NO: 390 - DNA, 261 bp, Homo sapiens
tcactcccacctgttactgctgggagtcaagtcagctaggaaggaagcaggacattttttcaaacagcaagtggggcccatggaactgaatctttactccttggtgcaccgcttttgtcgtgcgttgccttgctccgtttttcccaaaaagcactggcttcatcaaggccaccgacgatttcctgagtgcactgggaaatttgggtataggtcaggcttggcagccttgatcccaggagagtactaatggtaacaagtcaa

SEQ ID NO: 391 - DNA, 74 bp, Homo sapiens
gtgaaagggaagtagaaccgaaacaagattagtcctgagttaacaatggctgcaagctggatacatggaattca

SEQ ID NO: 392 - DNA, 248 bp, Homo sapiens
aaaaactcaacctatctggtgttttattttaatggataaaaatgtaatttttttaaggtagcaacttatttccaaattaatatagatgaaaaatagataccaattagactaaattgaaagctttttgttctatatttgcatagcctttgaaatatttcttagtgcctaggaggtctggggattcctctttcgtggtggtcactaaccttacttgatgcagataaaatcacttgtcaatgcaaaatgtg

SEQ ID NO: 393 - DNA, 286 bp, Homo sapiens
gctgtgttggccctcacttgggattctcagcagttacatgaaagttgtgctgataatctcttctcttgtaccaattttagtcaggcagaaaatggtaaacatgagggtgctcttgtgacttaatttttgttcaagggactaaattgcttatgtttattccctgtcagcggagtggagaatgtcattcatcaataaaccaaagccaatagctggagaattgagatctggttgaaagtggtttatggtttacatgctgtactatcctgaggaattgcgagatattgct

SEQ ID NO: 394 - DNA, 246 bp, Homo sapiens
tgtacagattcaagcaatggatgcaaggaacatgctgtatgtaatagaagaaagaagtccacgttttcggcagaagtagtgagtcagtgtggaagagaggtgagggtgtgctttactttttgataaagagaaagatgtttactcataaacccttcaaaaggtattaacaaatgtttaccaaacctattgctttattttaaaaacataatttgtgttttctatttgtaagatctgacatttcgaggc

SEQ ID NO: 395 - DNA, 110 bp, Homo sapiens
tttaacttgagggtgtagaggtcctccacgcttgtttgcctgaaagtaatataatgatgctgtctgaacaggttttactgcttgctttccaagtaaaggttaattatgat

SEQ ID NO: 396 - DNA, 251 bp, Homo sapiens
agagagtggacatttgtcgggaaactcctaacatatgcccccattctggagagaacacagagtacgacacaatccctcacactaatagaacaatcctaaaggaagatccagcaaatacggtttactccactgtggaaataccgaaaaagatggaaaatccccactcactgctcacgatgccagacacaccaaggctatttgcctatgagaatgttatctagacagcagtgcactgcccctaagtctctgct

SEQ ID NO: 397 - DNA, 266 bp, Homo sapiens
gcttggggaacaatgatggtgcacaaaggcttagatttgccttgtctcaaaataaggaattttgtagtggttttcaaaaataattcaacaaagaaacaatacaaaaagtgggtagaattacctatcacatttcccaatcttgactattcagaatgctgtttatttagtgatgaggattagcacttgattgaagattcttttaaaatactatcagttaaacatttaatatgattatgattaatgtattcattatgctacagaactga

SEQ ID NO: 398 - DNA, 165 bp, Homo sapiens
tttcctgccccaaggcagatccacatcaccgaagctccctagaggggcaaaagatggagtgagccacaggaagtttggggcgtggtgagttggaatgatacgtccatttctctatgaaatatttgctactagactgttcatttctctctgacatgtttgttgaat

SEQ ID NO: 399 - DNA, 275 bp, Homo sapiens
acttacatactagcttccaaggacaggtggaggtagggccagcctggcgggagtggagaagcccagtctgtcctatgtaagggacaaagccaggtttaatggtactgggtagggggcactgccaagacaataagctaggctactgggtccagctactactttggtgggattcaggtgagtctccatgcacttcacatgttacccagtgttcttgttacttccaaggagaaccaagaatggctctgtcacactcgaagccaggtttgatcaataaa

SEQ ID NO: 400 - DNA, 115 bp, Homo sapiens
aggagctgaggtgctacccggagccccattcacccccacctgcccacttgggaatctgaggcagaggagggtgaggcctgtgtgccaaccttgttcacataccaccttcgtcccc

SEQ ID NO: 401 - DNA, 114 bp, Homo sapiens
ctgcacagctcagcacaacattccaagctcaaaatagaagccttctcagtgagctccagcacgcccagaggactgttaataacgatgatccatgtgttttactctaaagtgcta

SEQ ID NO: 402 - DNA, 105 bp, Homo sapiens
atttccatcacatatgtgccaagacttgtgttctgtatccaggagtgtgttagatactaacatagtgtttcatttacatgtgtgtgaaacctgggtgaagagcca

SEQ ID NO: 403 - DNA, 173 bp, Homo sapiens
gtaccatcttacatgcttaaataactccacatttatttgtgtttattactctgtgttataaatatacatttgttggtctctctcttggattattttgtttctttgtcctgtaactaccactgaaagggtgcaatacagctttcttgaaatgtgtattgaacggatgaatgtat

SEQ ID NO: 404 - DNA, 262 bp, Homo sapiens
tggtgaccagttctcggtttcatagtttttactatcagtttgcctttgggattctttgaaagctcttgaggctttttccgcagcttctaggagatgtgttaggtcattaacagtaatgctcctacagtttttgttcccatccaaccaccatttgatttcacttttgtagacttgacctagtgtatctgaaatataggaatttttaggtgctttcattttggcctgacgtgcccagtccagagctgtgttaaagtccttctct

SEQ ID NO: 405 - DNA, 226 bp, Homo sapiens
gtcattcacaactgatttcaagagtcaccttcaccaggaagtcttccttgaccaccatcattcctgcctgattagagggcttcctcatggtaatatgtgttctcaagttttcagtgtcaaggaatgccatcccagaagctcattttcagatgcacaacagccagaacagtctcaagcagcattctagagcttggaatttaagaactacgcattgcctataaagtga

SEQ ID NO: 406 - DNA, 286 bp, Homo sapiens
actttgccggcgagcacaccgcctacccgcacggctgggtggagacggcggtcaagttggcgctgcgcgccgccatcaagatcaacagccggaaggggcctgcatcggacacggccagccccgaggggcacgcatttgacatggaggggcaggggcatgtgcatggggtggccagcagcccctcgcatgacctggcaaaggaagaaggcagccaccctccagtccaaggccagttatctttccaaaacacgacccacacgaggacctcgcattaaagtattttcgg

SEQ ID NO: 407 - DNA, 266 bp, Homo sapiens
  misc_feature (214)..(214): n is a, c, g, or t
ccctgcgcctatcaggtcgtgagtccaggggtctacaagtcccgggccccccagttcacgattctggcgcggacttcgctcccccaagacaacactcggaagccagggcccgcggcctacaacgtggatcagcaccggaagccccgcggctggagtttcgggatccggcactcggactacctggccccgctggtgaccgacgcggacaactganccgccaggcgggagcggccccacacgtgtttgcttaaagtctgcgagtccgc

SEQ ID NO: 408 - DNA, 123 bp, Homo sapiens
  misc_feature (52)..(52): n is a, c, g, or t
gcggactcgcagactttaagcaaacacgtgtggggccgctcccgcctgggtngcagttgtccgcgtcggtcaccagcggggccaggtagtccgagtgccggatcccgaaactccagccgcggg

SEQ ID NO: 409 - DNA, 109 bp, Homo sapiens
aagagaagactcacagtatcaggtctactgaaggagatacggtgattcctgttcttggctttgtagattcatctggtataaacagcactcctgagttatgaccttttga

SEQ ID NO: 410 - DNA, 173 bp, Homo sapiens
gctgagtatgttaagctctttatgactgtttttgtagtggtatagagtactgcagaatacagtaagctgctttattgtagcatttcttgatgttgcttagtcacttatttcataaacaacttaatgttttgaataatttcttactaaacattttgttattgggcaagtgattg

SEQ ID NO: 411 - DNA, 197 bp, Homo sapiens
agtgctaaggagtatagcagatgacttatatgtgtgttggctgggagaatatcatcttaaagtgagagtgatgttgtggagacagttgaaatgtcagtgctagagcctctgtggtgtgaatgggcacgttaggttgttgcattagaaagtgactgtttctgacagaaatttgtagctttgtgcaaactcacccacca

SEQ ID NO: 412 - DNA, 241 bp, Homo sapiens
ttgttcccgactagctgccttgcacattattttcattttcctggaatttgatacagagagcaatttatagccaattgatagcttatgctgtttcaatgtaaattcgtggtaaataacttaggaactgccttttctttttctttgaaaacctacttataactgttgctaataagaatgtgtattgttcaggacaacttgtctccatacagttgggttgtaaccctcatgcttggcccaaata

SEQ ID NO: 413 - DNA, 272 bp, Homo sapiens
caacctcttacaacccaggcattcctttctatcgataattactctttcaaccaattgccaatcagaaaattgttatatctacctataatctagaagcccccacatcaagttgttttgcctttctggacaggaccaatgtatatcttaaatgtatttgattgatctctcatgtctccctaaaatgtataaaaccacgctgttccccgaccacctggagcacatgttctcagggtctcctgagggctgtgtcacaggccatgttcacttacatt

SEQ ID NO: 414 - DNA, 203 bp, Homo sapiens
ttttcccaactctatagctagatttaaaagtcccagtaaaattttgtaaacaaaatcatataagaaaaggcaaggctggctcttccctatggtcctttagtggagctatatttgcatagatcctagacaaatgatgcaaaacaaattccctccaatttccactagcaatctccctaattcgctcaacccttacataagcatca

SEQ ID NO: 415 - DNA, 146 bp, Homo sapiens
atgttcataggttctcaaccctcacccccaccacgggagactagagctgcaggatcccaggggaggggtctctcctcccaccccaaggcatcaagcccttctccctgcactcaataaaccctcaataaatattctcattgtcaagg

SEQ ID NO: 416 - DNA, 149 bp, Homo sapiens
aatgttcataggttctaaaccctcaccccccccacgggagactagagctgcaggatcccaggggaggggtctctcctcccaccccaaggcatcaagcccttctccctgcactcaataaaccctcaataaatattctcattgtcaatcag

SEQ ID NO: 417 - DNA, 64 bp, Homo sapiens
atgttcataggttctcaaccctcaccccccaccacgggagactagagctcaggatcccagggga

SEQ ID NO: 418 - DNA, 152 bp, Homo sapiens
aatgttcataggttctaaaccctcaccccccccacgggagactagagctgcaggatcccaggggaggggtctctcctcccaccccaaggcatcaagcccttctccctgcactcaataaaccctcaataaatattctcattgtcaatcagcaa

SEQ ID NO: 419 - DNA, 151 bp, Homo sapiens
atgttcataggttctcaaccctcaccccccccacgggagactagagctgcaggatcccaggggaggggtctctcctcccaccccaaggcatcaagcccttctccctgcactcaataaaccctcaataaatattctcattgtcaatcagcaa

SEQ ID NO: 420 - DNA, 254 bp, Homo sapiens
gcagctatttgagcctgacgcctgagcagtggaagtcccacagaagctacagctgccaggtcacgcatgaagggagcaccgtggagaagacagtggcccctacagaatgttcataggttctaaaccctcaccccccccacgggagactagagctgcaggatcccaggggaggggtctctcctcccaccccaaggcatcaagcccttctccctgcactcaataaaccctcaataaatattctcattgtcaatcag

SEQ ID NO: 421 - DNA, 66 bp, Homo sapiens
ggagaaccaccccacccccaatgtccaccatgaccctcttcccaacgctgaacctgtgctccctcc

SEQ ID NO: 422 - DNA, 147 bp, Homo sapiens
atgttcataggttctcaaccctcaccccccaccacgggagactagagctgcaggatcccaggggaggggtctctcctcccaccccaaggcatcaagcccttctccctgcactcaataaaccctcaatatatattctcattgtcaatc

SEQ ID NO: 423 - DNA, 212 bp, Homo sapiens
gatgcatgaggctctgcacaaccactacacgcagaagagcctctccctgtctccgggtaaatgagtgcgacggccggcaagcccccgctccccaggctctcggggtcgcgcgaggatgcttggcacgtaccccgtgtacatacttcccgggcgcccagcatggaaataaagcacccagcgctgccctgggcccctgcgcaactttcttgtac

SEQ ID NO: 424 - DNA, 236 bp, Homo sapiens
actctcaggctgcgtccagcgacagtgcccagggctctgatgtgtctctcacagcttgaaaagcctgagacagctgtcttgtgagggactgagatgcaggatttcttcacgcctcccctttgtgacttcaagagcctctggcatctctttctgcaaaggcacctgaatgtgtctgcgtccctgttagcataatgtgaggaggtggagagacagcccacccttgtgtccactgtgac

SEQ ID NO: 425 - DNA, 92 bp, Homo sapiens
gctgagcaaagcagactacgagaaacacaaagtttacgcctgcgaagtcacccatcagggcctgagctcgcccgtcacaaagagcttcaaca

SEQ ID NO: 426 - DNA, 174 bp, Homo sapiens
  misc_feature (27)..(27): n is a, c, g, or t
  misc_feature (78)..(78): n is a, c, g, or t
  misc_feature (96)..(96): n is a, c, g, or t
  misc_feature (123)..(123): n is a, c, g, or t
acgagccctctcacagtggaatggagngcacggtctgaatctgcacagagcaagatgctgagtggagtcgggggcttngtgctgggcctgctcttncttggggccgggctgttcatctacttnaggaatcagaaaggacactctggacttcagccaacaggattcctgaactga

SEQ ID NO: 427 - DNA, 150 bp, Homo sapiens
aatgttcataggttctcaaccctcaccccccaccacgggagactagagctgcaggatcccaggggaggggtctctcctcccaccccaaggcatcaagcccttctccctgcactcaataaaccctcaataaatattctcattgtcaatcag

SEQ ID NO: 428 - DNA, 150 bp, Homo sapiens
aatgttcataggttctcatccctcaccccccaccacgggagactagagctgcaggatcccaggggaggggtctctcctcccaccccaaggcatcaagcccttctccctgcactcaataaaccctcaataaatattctcattgtcaatcaa

SEQ ID NO: 429 - DNA, 150 bp, Homo sapiens
aatgttcataggttctcaaccctcaccccccaccacgggagactagagctgcaggatcccaggggaggggtctctcctcccaccccaaggcatcaagcccttctccctgcactcaataaaccctcaataaatattctcattgtcaatcaa

SEQ ID NO: 430 - DNA, 95 bp, Homo sapiens
ccagacctacacctgcaacgtagatcacaagcccagcaacaccaaagtggacaagacagttgagcgcaaatgttgtgtcgagtgcccaccgtgcc

SEQ ID NO: 431 - DNA, 41 bp, Homo sapiens
tacaagaccacgcctcccgtgctggactccgacggctcctt

SEQ ID NO: 432 - DNA, 181 bp, Homo sapiens
  misc_feature (43)..(44): n is a, c, g, or t
  misc_feature (78)..(79): n is a, c, g, or t
  misc_feature (99)..(99): n is a, c, g, or t
  misc_feature (110)..(110): n is a, c, g, or t
gccagagaagcgattagaaacccctgagggccgattactgacnncataaatcatgagtttgggggctttgcctgggtnntgttggtaccaggagacatngttataaccancaacgtcactgctggttccagtgcaggagatggtgatcgactgtccaggagacccagacacggaggcaggc

SEQ ID NO: 433 - DNA, 131 bp, Homo sapiens
ctacttccccgaaccggtgacggtgtcgtggaactcaggcgccctgaccagcggcgtgcacaccttcccggctgtcctacagtcctcaggactctactccctcagcagcgtggtgaccgtgccctccagca

SEQ ID NO: 434 - DNA, 298 bp, Homo sapiens
agcagactacgagaaacacaaagtttacgcctgcgaagtcacccatcagggcctgagctcgcccgtcacaaagagcttcaacaggggagagtgttagagggagaagtgcccccacctgctcctcagttccagcctgaccccctcccatcctttggcctctgaccctttttccacaggggacctacccctattgcggtcctccagctcatctttcacctcacccccctcctcctccttggctttaattatgctaatgttggaggagaatgaataaataaagtgaatctttgcacctgtg

SEQ ID NO: 435 - DNA, 186 bp, Homo sapiens
gctgagcaaagcagactacgagaaacacaaagtctacgcctgcgaagtcacccatcagggcctgagctcgcccgtcacaaagagcttcaacaggggagagtgttagagggagaagtgcccccacctgctcctcagttccagcctgaccccctcccatcctttggcctctgaccctttttccacagg

SEQ ID NO: 436 - DNA, 172 bp, Homo sapiens
agccaatggaaaatctgggttcaaccagcccctgccatttcttaagactttttgctgcactcacaggatcctgagctgcacttacctgtgagagtcttcaaacttttaaaccttgccagtcaggacttttgctattgcaaatagaaaacccaactcaacctgcttaagcaga

SEQ ID NO: 437 - DNA, 223 bp, Homo sapiens
tggtcatgagaagaaggtaatttccagccttcaagaagacagacatttagaagaagagctgaaatgtcaggaacaaaaagaagaacagctgcaggaaggggtgcaccggaaggagccccagggggccacgtagcagcggctcagtgggtggccatcgatttggaccgtcccctgcccacttgctccccgtgagcactgcgtacaaacatccaaaagttcaaca

SEQ ID NO: 438 - DNA, 283 bp, Homo sapiens
gtgagaggtaacactctaaatagcccatttcatgctcaagacatccaagtcaaagaaacccaatagcacaggtgagtcccctctgttcccccccaacaccccactcacatcagggcccctgccctggagtgtcacctttattagctgtgagagacaccccagagccctgggcactgtcagtgattggggtagaacaaaaacaggacctggtcagagcccacagatgtggctagaggaactgtggggtggtgagctccctcataggctcctgaccacaatatcc

SEQ ID NO: 439 - DNA, 174 bp, Homo sapiens
gatcagcaaacaggaatacgatgaagccgggccttccattgtccaccgcaaatgcttctaaaacactttcctgctcctctctgtctctagcacacaactgtgaatgtcctgtggaattatgccttcagttcttttccaaatcattcctagccaaagctctgactcgttacctat

SEQ ID NO: 440 - DNA, 72 bp, Homo sapiens
acatgagtatggaatggtgttttattatgactttagtttgcattttcctcaattctcgttaaatccttcatt

SEQ ID NO: 441 - DNA, 181 bp, Homo sapiens
tggtgacagagtttgggaccgaggtggagcccgagtttgggaccaaggtggagcccgagtttgagacccagttggagcctgagtttgagacccagctggaacccgagtttgaggaagaggaggaggaggagaaagaggaggagatagccactggccaggcattccccttcacaacagtaga

SEQ ID NO: 442 - DNA, 279 bp, Homo sapiens
gtaggtttgggagtataacggtcacccagggagggggtgaagacggagaagacttacatagcacggtcaggttagggctggacagatgaggaagagctagcaaagggggcttgaggagcagtggccactaagacaggagtgtgacattttagaagccaaaagaagaccatgtaattcaagggagaggtatgatttgctgggtcagatctaaaaataaatcacacgtttttttaaactgtagtaattaaccactgaaaacttatgagtgatccaatatta

SEQ ID NO: 443 - DNA, 275 bp, Homo sapiens
gtaggtttgggagtataacggtcacccagggagggggtgaagacggagaagacttacatagcacggtcaggttagggctggacagatgaggaagagctagcaaagggggcttgaggagcagtggccactaagacaggagtgtgacattttagaagccaaaagaagaccatgtaattcaagggagaggtatgatttgctgggtcagatctaaaaataaatcacacgtttttttaaactgtagtaattaaccactgaaaacttatgagtgatccaat

SEQ ID NO: 444 - DNA, 197 bp, Homo sapiens
atgtcaagaaaatgacggtcacagaccaggtgaactgccccaagctctcgtaaccaggttctacagggaggctgcacccactccatgttacttctgcttcgctttcccctaccccaccccccccccataaagacaaaccaatcaaccacgacaaaggaagttgacctgaacatgtaaccatgccctaccctgttacc

SEQ ID NO: 445 - DNA, 76 bp, Homo sapiens
ggaggatgcgctgtggggttgtttttgccataagcgaactttgtgcctgtcctagaagtgaaaattgttcagtcca

SEQ ID NO: 446 - DNA, 173 bp, Homo sapiens
gaaggagaacgaatccagtggaaagaaagctggtgagggaaaagatgccctgcagaaaagtcccctttcccccactgtctggacactggtgaatgacattagaagagacccaccccattcaagtcccctcactggctccttttctccccactacaccacttccaaaatctgaa

SEQ ID NO: 447 - DNA, 169 bp, Homo sapiens
gagaagtcactcacactggccacaaggacgctggctactgtctattaaaattttgatgtttctgtgaaattctcagagtgtttaattgtactcaatggtatcattacaattttctgtaagagaaaatattacttatttatcctagtattcctaacctgtcagaataata

SEQ ID NO: 448 - DNA, 38 bp, Homo sapiens
tttcccccactgtctggacactggtgaatgacattaga

SEQ ID NO: 449 - DNA, 275 bp, Homo sapiens
acacaatacaatcaggagttttcaaatttttgattcagtatttgaatttcttcttcataaatgtagttggaatttatcctagtatttttctttacctgaaggagggccatttatttttaatttcactacatttttctttgcatgattattaaaataaaaactgcctctgttgtgtttctcactggaggctggaatgaatgatcactagaacacaaaagagtgaatgatgacacttgaagtcaaagcagttgtactgatcaccagaaccaataaag

SEQ ID NO: 450 - DNA, 273 bp, Homo sapiens
gactgagtgagtaactgcaagctacagggggcagcttacgtatggcacacagggtggggctagcttggtttcataggctctctgtggattggatgatttaaataattttggtgggccctggggtttagggactgtccctagttgtttggtgctaggtcccagggcagattagggcagatgtgagtgtgagagcatgataaggaaagtcttcaaggtgtggaattactcaactgctggagaaagggaatttatcagcctttagccagggcctca

SEQ ID NO: 451 - DNA, 93 bp, Homo sapiens
tggagaagcactggtgtctgcagcacccctcagttcctgtgcctcagcccacaggccactgtgataatggtctgtttagcacttctgtattta

SEQ ID NO: 452 - DNA, 111 bp, Homo sapiens
ttaagacattggagtgatttctggaaatgttttctttaagaaggctcacgtgatgtttgtgtttacttgtggttgccctatcctatgctgcataaatccttgaaaggaaag

SEQ ID NO: 453 - DNA, 137 bp, Homo sapiens
ttaagacattggagtgatttctggaaatgttttctttaagaaggctcacgtgatgtttgtgtttacttgtggttgccctatcctatgctgcataaatccttgaaaggaaaggttttagttagttgctttctttcttc

SEQ ID NO: 454 - DNA, 148 bp, Homo sapiens
  misc_feature (89)..(89): n is a, c, g, or t
  misc_feature (91)..(93): n is a, c, g, or t
gtcattgatacctgtagtaagttgacaatgtggtgaaatttcaaaattatatgtaacttctactagttttactttctcccccaagtctntnnnaactcatgatttttacacacacaatccagaacttattatatagcctctaagtctt

SEQ ID NO: 455 - DNA, 257 bp, Homo sapiens; identical to SEQ ID NO: 19

SEQ ID NO: 456 - DNA, 119 bp, Homo sapiens
actttaataaaggttatccataccaataaaaagtgtacaacacagcattttctgttaaattattattggttttcagttgtaatttggtattttttctggcatgcgtttattaatttatt

SEQ ID NO: 457 - DNA, 198 bp, Homo sapiens
gattttcttttgacacagctccaaggccaccagctatgcaaggccacaagttatgcactatatgattaactgcttttgttttacttttgtaagtccacttataaaaaccctgctctgtctttgtttaatgctcagctttttggatttgaatccactcagccggtgcacaccttaaaataaacatcctcctgtactctc

SEQ ID NO: 458 - DNA, 38 bp, Homo sapiens
tttggatttgaatccactcagccggtgcacaccttaaa

SEQ ID NO: 459 - DNA, 53 bp, Homo sapiens
gaccaggaattcggcttcgacgttggccctgtctgcttcctgtaaactccctc

SEQ ID NO: 460 - DNA, 37 bp, Homo sapiens
tgggctggagccgcacacgctctcctcccatgttaaa

SEQ ID NO: 461 - DNA, 199 bp, Homo sapiens
  misc_feature (113)..(113): n is a, c, g, or t
  misc_feature (115)..(115): n is a, c, g, or t
  misc_feature (122)..(122): n is a, c, g, or t
aagggagccgggaggatgggctgcagctgtggaggagggtttcagaggagagaggtcggagagcagaggcctgagaagccagaggcaggtggagagagggtggaaagtgagcntnagcgggncttgggctggagccgcacacgctctcctcccatgttaaatagcacctttagaaaaattcacaagtccccatccacat

SEQ ID NO: 462 - DNA, 211 bp, Homo sapiens; identical to SEQ ID NO: 43

SEQ ID NO: 463 - DNA, 211 bp, Homo sapiens; identical to SEQ ID NO: 33

SEQ ID NO: 464 - DNA, 286 bp, Homo sapiens; identical to SEQ ID NO: 48

SEQ ID NO: 465 - DNA, 275 bp, Homo sapiens; identical to SEQ ID NO: 46

SEQ ID NO: 466 - DNA, 261 bp, Homo sapiens
agccgctgccaagtctgtatgagaagaacataatgaagcctgactcagctaatgtcacaacatggtgctacttcttcttctttttgttaacagcaacgaaccctagaaatatatcctgtgtacctcactgtccaatatgaaaaccgtaaagtgccttataggaatttgcgtaactaacacaccctgcttcattgacctctacttgctgaaggagaaaaagacagcgataagctttcaatagtggcataccaaatggcactt

SEQ ID NO: 467 - DNA, 238 bp, Homo sapiens; identical to SEQ ID NO: 203

SEQ ID NO: 468 - DNA, 251 bp, Homo sapiens; identical to SEQ ID NO: 210

SEQ ID NO: 469 - DNA, 270 bp, Homo sapiens; identical to SEQ ID NO: 53

SEQ ID NO: 470 - DNA, 245 bp, Homo sapiens; identical to SEQ ID NO: 57

SEQ ID NO: 471 - DNA, 221 bp, Homo sapiens; identical to SEQ ID NO: 59

SEQ ID NO: 472 - DNA, 129 bp, Homo sapiens; identical to SEQ ID NO: 27

SEQ ID NO: 473 - DNA, 94 bp, Homo sapiens; identical to SEQ ID NO: 26

SEQ ID NO: 474 - DNA, 202 bp, Homo sapiens
taatggttaacgaaccgggtcgacatcacaaaggagggtggagactctttttactaacttgaatgagacaaaagcagtggtgtcagtttataatcctgatgcatttcagtaataatgtagaaaaacattattttaaaaaagttccaacacacagccatgaggagcctcagttttgaaagaggtgcataataaaactactaac

SEQ ID NO: 475 - DNA, 243 bp, Homo sapiens
ggattttttcccctgtagtagtgaggtaacatgcttgaatgtcactgtgatatttatttcctctttgttcagttgtttttgaattcctgttaagtacatgttttaatactttgagcgatttaagatacttttctttttgcccatcattttccccaaggaatgtaattcacataaatccaaagctcatttttttttttattgtacacaagtagtataatgtttgcttttcccaataaacctcaa

SEQ ID NO: 476 - DNA, 137 bp, Homo sapiens
ggattttttcccctgtagtagtgaggtaacatgcttgaatgtcactgtgatatttatttcctctttgttcagttgtttttgaattcctgttaagtacatgttttaatactttgagcgatttaagatacttttctttt

SEQ ID NO: 477 - DNA, 112 bp, Homo sapiens
attttagtatggtgtctgtttatgtaactctgacttgctggaaaagttgaaactccaaataatctgaaactagaaaagaaatagcacataattactaccttccccttggcgg

SEQ ID NO: 478 - DNA, 207 bp, Homo sapiens
aagtgttctacaaaagaatccctgtggttagcttactcttaggttagatcttctaataaggctgaaattcaaaatcaaaaccttagtgtgtccgagtccagcctgggttccagcattctgttcaggccacttctgaacggccgaaggtgccccattccagacctgcccatttgatggacagagcagacagcccggaacagattcaag

SEQ ID NO: 479 - DNA, 237 bp, Homo sapiens; identical to SEQ ID NO: 181

SEQ ID NO: 480 - DNA, 113 bp, Homo sapiens; identical to SEQ ID NO: 185

SEQ ID NO: 481 - DNA, 146 bp, Homo sapiens
gctttgatatttcaatgttagcctcaatttctgaacaccataggtagaatgtaaagcttgtctgatcgttcaaagcatgaaatggatacttatatggaaattctgctcagatagaatgacagtccgtcaaaacagattgtttgcaa

SEQ ID NO: 482 - DNA, 179 bp, Homo sapiens
tgtggtagcctcacttttaatgaacaaatggcctttattaaaaactgagtgactctatatagctgatcagttttttcacctggaagcatttgtttttactttgatatgactgtttttcggacagtttatttgttgagagtgtgaccaaaagttacatgtttgcacctttttagttgaaa

SEQ ID NO: 483 - DNA, 115 bp, Homo sapiens
tgaacaaatggcctttattaaaaactgagtgactctatatagctgatcagttttttcacctggaagcatttgtttttactttgatatgactgtttttcggacagtttatttgttg

SEQ ID NO: 484 - DNA, 221 bp, Homo sapiens
ttgctggggcttgtcctagaggctccagctttggcacagtggttcctggctgctgccatgtttcagatgaggagggagagaaggaggccgccagactcgagaggtgggaggaactccttgcacacaccctgagcttttgccacttttatcatttttgagcaactccctttcagctaaaaggccacccctttatcgcattgctgtccttgggtagaatataa

SEQ ID NO: 485 - DNA, 73 bp, Homo sapiens
tctgtaattcattgagcagttagctcatttgagataaagtcaaatgccaaacactagctctgtattaatcccc

SEQ ID NO: 486 - DNA, 55 bp, Homo sapiens
atactggaaacctaactgcaatgtggatgttttacccacatgacttattatgcat

SEQ ID NO: 487 - DNA, 164 bp, Homo sapiens
gtgtggatgctaaggtgtttgttttgttttgtatttttatgtagcgcgtgggtattgtgcctagaaatgaagtcattattagggatttaaatatgcaactcatggagtggatgagaccagctagaaagataatagagtgtgaagaggagatcggaaattcaata

SEQ ID NO: 488 - DNA, 233 bp, Homo sapiens
  misc_feature (35)..(35): n is a, c, g, or t
  misc_feature (53)..(53): n is a, c, g, or t
  misc_feature (60)..(60): n is a, c, g, or t
tggagtaacacccagatctctgcagcagttaagcntgggggcctagaactagnctagagntagaagaagggacaaatgcaatccgacctttggatctacacattcctcttgcttcaatgggtgtcatttaagaattagaggaaaatattaggagatggagaactagagttgaggaaaccaaaagaagaggagtcacagaaaaccagctctctctgtgcaaggcatcttgaaag

SEQ ID NO: 489 - DNA, 195 bp, Homo sapiens
tatgagttattcaaggaggagactttttaaagacagcaacgcaattcttgtaacttgtgtaaatagccccatctttcagagtgataccatttctacatttgataatgcctgtattcctgtaggatgtatatagtttaggggattttttttttgtttggttttgttttttagaagtcaatatgtctggttttattt

SEQ ID NO: 490 - DNA, 268 bp, Homo sapiens
atgagtcaaaatccgctctccatgcttactcttgacaccccattgaagccactcattgtgtgtgcgtctgggtgtgaagtccagctccgtgtggtcctgtgcttgtactgccctgctttgcagttcctttgcacttactcatcgagtgctgttttgaaatgctgacattatataaacgtaaaagaaaatgtaaaaaaaaaaaacccacacacaaacaaacccatacgatctgtatttgtatatacacgtgtccgtacaagtataacta

SEQ ID NO: 491 - DNA, 289 bp, Homo sapiens
tcccaagtagtgctgactgactctcctggtgacaggggtttgtgtccgagcccctgcggtcaaggagtgtggagcaaaacgtgggtactagggtgggaggcggggaaaggccacagcacactggcgctccagcaaagccaaatcatgtctcctctggccactgcggtcctctccttggtacatgtcatcccccagaggagtatccaaagctattccactatgcactcatcaaccctggcttgtcagccttggggaaggtcactttattcataaaaatgcctctttgagt

SEQ ID NO: 492 - DNA, 151 bp, Homo sapiens
ttggaatgttgtagttacctactgagtaggcggcgatttttgtatgttatgaacatgcagttcattattttgtggttctattttactttgtacttgtgtttgcttaaacaaagtgactgtttggcttataaacacattgaatgcgctttat

SEQ ID NO: 493 - DNA, 178 bp, Homo sapiens
ctttggatagaggtgaagaacttggacatggctgtttcaggcagctgaagtcaaagggaatagtaattggggaaggggaagtgggcagaaaggattgttggccaatataccttccactccagtagagagggaggacttggctctgagaacctccatctgacctaagaggaaccctcct

SEQ ID NO: 494 - DNA, 100 bp, Homo sapiens
tgccccccttaaggctagaggtgagcatgtccctcacaattgcacatgtcaagccatcagcaaggcgcatcacacaaaaggcaccaagacgtgaaacttt

SEQ ID NO: 495 - DNA, 156 bp, Homo sapiens
ctgagtaaatgggactgctgtcgttggatggcactgcgcagctcaggggtgggctctgggaggcggaggcggaggaggccgctggagatggtgctgaggacgaggaggccggtgggttggtcatgctcactaggccactgaccaagctgaagaggg

SEQ ID NO: 496 - DNA, 233 bp, Homo sapiens
gaaacacaagagacttaaaggacaggaggaggagatggccataggagaggagggttcctcttaggtcagatggaggttctcagagccaagtcctccctctctactggagtggaaggtctattggccaacaatcctttctgcccacttccccttccccaattactattccctttgacttcagctgcctgaaacagccatgtccaagttcttcacctctatccaaagaacttgat

SEQ ID NO: 497 - DNA, 108 bp, Homo sapiens
acatgcagtactgtataccccccatccctccctcggtccactgaacttcagagcagttcccattcctgccccgcccatctttttgtgtctcgctgtgatagatcaata

SEQ ID NO: 498 - DNA, 222 bp, Homo sapiens
atgattatagaaggctgtcttagtgcaaaaaacatacttacatttcagacatatccaaagggaatactcacattttgttaagaagttgaactatgactggagtaaaccatgtattcccttatcttttactttttttctgtgacatttatgtctcatgtaatttgcattactctggtggattgttctagtactgtattgggcttcttcgttaatagattattt

SEQ ID NO: 499 - DNA, 269 bp, Homo sapiens
tgctggcactgatattatccatcatctctttttggacacttctgtaaatgtgattggattgtttgaaagaagatttaaagtttcaaagttttttgttctgtttttgctttgcatttggagaaaatattgaaagcagggtatgttgtttcattcaccttgaaaaaaccatgagtaaatggggatatagaatctctgaatagctcgctaaaagattcaagcaagggacatgaattttgttccatctatcaataatatccagaagaacaact

SEQ ID NO: 500 - DNA, 174 bp, Homo sapiens
gtttgaggtatttaactgacacctaagtggatctgttgaggaaacagttggatatacaaatttagtgtttaaggcagacttccaggcttgaaggaaaaatttggaagtcatcacgacatatatgtggtatttaaaattgtgaggttcaaggaccaagccccataccatttagag

SEQ ID NO: 501 - DNA, 232 bp, Homo sapiens
tttgttagtacatttcagtgtagtcattcatttctagctgtacataggatgaaggagagatcagatacatgaacatgttttacatgggttgctgtatttagaattataaacatttttcattattggaaagtgtaacggggaccttttgcatacctgtttagaaccaaaaccaccatgacacagtttttatagtgtctgtatatttgtgatgcaatggtcttgtaaaggtttt

SEQ ID NO: 502 - DNA, 36 bp, Homo sapiens
aaaaaagttcaactagtatgaaagggttataaagta

SEQ ID NO: 503 - DNA, 96 bp, Homo sapiens
catatattttttgctacttttgctgttttatttttttaaattatgttctaaacctattttcagtttaggtccctcaataaaaattgctgctgcttc

SEQ ID NO: 504 - DNA, 122 bp, Homo sapiens
aggtggtgttggcagaggctatcgggctggacaaggacaaacccaaccgtgtgaccaaagtggctgtgaagatgttgaagtcggacgcaacagagaaagacttgtcagacctgatctcagaa

SEQ ID NO: 505 - DNA, 286 bp, Homo sapiens
aactgggggctctgtgggggctctgtatatagctatgaagaaaacacaaagtgtataaatctgagtatatatttacatgtctttttaaaagggtcgttaccagagatttacccatcgggtaagatgctcctggtggctgggaggcatcagttgctatatattaaaaacaaaaaagaaaaaaaaggaaaatgtttttaaaaaggtcatatattttttgctacttttgctgttttatttttttaaattatgttctaaacctattttcagtttaggtccctcaataaaa

SEQ ID NO: 506 - DNA, 268 bp, Homo sapiens
gtaaagctttggcacatacagtataaaaaataatcacccaccataattataccaaattcctcttatcaactgcatactaagtgttttcaatacaattttttccgtataaaaatactgggaaaaattgataaataacaggtaagagaaagatatttctaggcaattactaggatcatttggaaaaagtgagtactgtggatatttaaaatatcacagtaacaagatcatgcttgttcctacagtattgcgggccagacacttaagtgaa

SEQ ID NO: 507 - DNA, 145 bp, Homo sapiens; identical to SEQ ID NO: 67

SEQ ID NO: 508 - DNA, 217 bp, Homo sapiens
gttcccagtgacacttcagagagctggtagttagtagcatgttgagccaggcctgggtctgtgtctcttttctctttctccttagtcttctcatagcattaactaatctattgggttcattattggaattaacctggtgctggatattttcaaattgtatctagtgcagctgattttaacaataactactgtgttcctggcaatagtgtgttctgat

SEQ ID NO: 509 - DNA, 133 bp, Homo sapiens
gtatggatgggtatgttgagactcaattacttttttattagcttccccgtttggaagatcccaaacaccaaagatggaaggtgaaaataaagactgcgtgaccgggaagaaagtttgaattactaatagtggg

SEQ ID NO: 510 - DNA, 183 bp, Homo sapiens
ggattataatatcctcactggccacaatctgtaaaagtcgatactggcactttttttgccccctcaaaggaaatatgctaatagacagcccctttgcaaatataattcctccttcccaacccttcaaattgctaaggccccactggtcagcaccttccctttcgagtccaggactactgttct

SEQ ID NO: 511 - DNA, 227 bp, Homo sapiens
tatttgaactatatgttgaagacatctaccagtttctccaaatgccttttttaaaactcatcacagaagattggtgaaaatgctgagtatgacacttttcttcttgcatgcatgtcagctacataaacagttttgtacaatgaaaattactaatttgtttgacattccatgttaaactacggtcatgttcagcttcattgcatgtaatgtagacctagtccatcaga

SEQ ID NO: 512 - DNA, 199 bp, Homo sapiens
gggagtcaatgaatagtacctaaaatggaaaccaaacaaaacaacttcaggaagtaacaagggcttgcttagagacatgacggtaaaccctgaaccatcagctaaaagaggtagatagcagtggttgcccctggggagaggtaaatgtgatggagagggaacaactgtgtacaaacatgtgactttacgttttgatcaa

SEQ ID NO: 513 - DNA, 172 bp, Homo sapiens
aactgtgaccgtttctgtgtgaagatttttagctgtatttgtggtctctgtatttatatttatgtttagcaccgtcagtgttcctatccaatttcaaaaaaggaaaaaaaagagggaaaattacaaaaagagagaaaaaaagtgaatgacgtttgtttagccagtaggagaa

SEQ ID NO: 514 - DNA, 121 bp, Homo sapiens
ttatatttatgtttagcaccgtcagtgttcctatccaatttcaaaaaaggaaaaaaaagagggaaaattacaaaaagagagaaaaaaagtgaatgacgtttgtttagccagtaggagaaaa

SEQ ID NO: 515 - DNA, 265 bp, Homo sapiens
tgttttagaaccagccgtattttacatgaagctgtataattaattgtcattatttttgttagcaaagattaaatgtgtcattggaagccatccctttttttacatttcatacaacagaaaccagaaaagcaatactgtttccattttaaggatatgattaatattattaatataataatgatgatgatgatgatgaaaactaaggatttttcaagagatctttctttccaaaacatttttggacagtacctgattgtattttttt

SEQ ID NO: 516 - DNA, 169 bp, Homo sapiens
tgttaaactttggaacacctaccaaaaaataagtttgataacatttaaaagatgggcgtttcccccaatgaaatacacaagtaaacattccaacattgtctttaggagtgatttgcaccttgcaaaaatggtcctggagttggtagattgctgttgatcttttatcaat

SEQ ID NO: 517 - DNA, 272 bp, Homo sapiens
tttcactcttgctgtctgctcctctcacatcatccttgcctctgtctgtttaatcctcctgtccttcattttccttttttgcctctgcattcagcatttctacttccaatctccctcctctgctctttcttatttcctctgatctgcagacttgcttctgtcccctccttctgttcccctcctggatgtgtctttggccaacctttccttctctgagacttcgtgttcttgttggtagatgggggctgatacttctgggtcttgggcatgtc

SEQ ID NO: 518 - DNA, 262 bp, Homo sapiens
tttcactcttgctgtctgctcctctcacatcatccttgcctctgtctgtttaatcctcctgtccttcattttccttttttgcctctgcattcagcatttctacttccaatctccctcctctgctctttcttatttcctctgatctgcagacttgcttctgtcccctccttctgttcccctcctggatgtgtctttggccaacctttccttctctgagacttcgtgttcttgttggtagatgggggctgatacttctgggtct

SEQ ID NO: 519 - DNA, 201 bp, Homo sapiens
ccggacagtggccttctccactcccctctgacttctccaagggggctcagtggccagtgccccccaggaggctccaccctcaactcaacccaagcaacagggacagatgaaaaacaaaatccaatcagggcgataaatggcggggggcaggacgtggtggtctccaggctggcttcgtgcgttcttgcttttgtcactgcc

SEQ ID NO: 520 - DNA, 237 bp, Homo sapiens
agaagacccacgtgctaggggatgaggggcttcctgggtcctgttccctaccccatttgtggtcacagccatgaagtcaccgggatgaacctatccttccagtggctcgctccctgtagctctgcctccctctccatatctccttcccctacacctccctccccacacctccctactcccctgggcatcttctggcttgactggatggaaggagacttaggaacctaccagttggcc

SEQ ID NO: 521 - DNA, 263 bp, Homo sapiens
aatcaaccaatgcaaccagtttgtgagaaaaaaaaaaaaaagccgaaaaaaaaaaaaaaaaacacctgaatgcggaagagctcggctcccgtttagcattttgtacttaaggaaataaaaaaccaacaaaggatttcacatttttttaaaaagtgaagattgctgtatactatttattcaacttataatttatgttactccttgatctttgtcttttgtcatgacaaagcatttatttaataaagttatgcattcagcaactt

SEQ ID NO: 522 - DNA, 300 bp, Homo sapiens
gatgataatctttactggtgaaaaggatggaaaaataaatcaacaaatgcaaccagtttgtgagaaaaaaaaaaaaaagccgaaaaaaaaaaaaaaaacacctgaatgcggaagagctcggctcccgtttagcattttgtacttaaggaaataaaaaaccaacaaaggatctcacattttcttaaaaagtgaagattgctgtatactatttattcaacttataatttatgttactccttgatctttgtcttttgtcatgacaaagcatttatttaataaagttatgcattcagttcccaa

SEQ ID NO: 523 - DNA, 33 bp, Homo sapiens
aaacgatgataatctttactggtgaaaaggatg

SEQ ID NO: 524 - DNA, 180 bp, Homo sapiens
acagtgattccccacgtgtgttcatctgcacccaccgagccaggcagaggccagccctccgtggtgcacacagcacgcgcctcagtccatcccattttagtctttaaaccctcaggaagtcacagtctccggacaccacaccacatgagcccaacaggtccacgatggatccaccagtcc

SEQ ID NO: 525 - DNA, 137 bp, Homo sapiens
gtgggcagcacttagattcggagccatggatagtccggagtccaaggtctctgggtgagcagacagtcggccaaaggccagcctggagtcaaagagaccagacccctgcttagattgccatactcgcaccattccaa

SEQ ID NO: 526 - DNA, 259 bp, Homo sapiens
gctggtgtgtggatcggatgggcaagtccctgccagggtctccagatggcaatggaagctcctcctgccccactgggagtagcggctaaagctgggggatagaggggctgcagggccactggaaggaacatggagctgtcatcactcaacaaaaaaccgaggccctcaatccaccttcaggccccgccccatgggcccctcaccgctggttggaaagagtgttggtgttggctggggtgtcaataaagctgtgcttggg

SEQ ID NO: 527 - DNA, 161 bp, Homo sapiens
aatgaaattgttaattggccaggcacagtgggaagcacctgtagtcccagctactcaggaggctaaagtgagagggtggctctagcacagtcatcaaggctgcaggaggctatgatggagccactgcactccagcctagatgacaaggtgagacgctgtct

SEQ ID NO: 528 - DNA, 249 bp, Homo sapiens
cggttgttaaaactggtttagcacaatttatattttccctctcttgccttttttatttgcaataaaaggtattgagccattttttaaatgacatttttgataaattatgtttgtactagttgatgaaggagttttttttaacctgtttatataattttgcagcagaagccaaattttttgtatattaaagcaccaaattcatgtacagcatgcatcacggatcaatagactgtacttattttccaataa

SEQ ID NO: 529 - DNA, 221 bp, Homo sapiens
cctgtaagacaataggccatgttaattaaactgaagaaggatatatttggctgggtgttttcaaatgtcagcttaaaattggtaattgaatggaagcaaaattataagaagaggaaattaaagtcttccattgcatgtattgtaaacagaaggagatgggtgattccttcaattcaaaagctctctttggaatgaacaatgtgggcgtttgtaaattctgg

SEQ ID NO: 530 - DNA, 138 bp, Homo sapiens
ttcccaagcccatgagtccttgaaaatattttttatatatacagtaactttatgtgtaaatacataagcggcgtaagtttaaaggatgttggtgttccacgtgttttattcctgtatgttgtccaattgttgacagtt

SEQ ID NO: 531 - DNA, 246 bp, Homo sapiens
tccaacaacagtgctgactgtccggataagagctggcaagtgcccttaggatgccgcatgggaaaaatcggttatcataatttcaaagtataaatatatttattatgtagcgctgcggttaagaaggaagagtgaggggtctgtccatggggtggcagtgatttcccacccgcctttcttgaatggcttcgtgttattcagccgtgccctggcaggaatggaactccatcagggaacagggcagct

SEQ ID NO: 532 - DNA, 165 bp, Homo sapiens; identical to SEQ ID NO: 113

SEQ ID NO: 533 - DNA, 103 bp, Homo sapiens
aaaccttgttttattcagcccagacctgggagatctagctctgtggtatggccaaagtataaaaaataaaaaccaacaacaacaaaacaacactgtagagaaa

SEQ ID NO: 534 - DNA, 243 bp, Homo sapiens
gggatctccttttgtgaaaaccagtttgatgtgctaaaagtaaaaagtctattttccagtgtggtcttgttcagaagcagccagatttccaatgttgtttttcccctccactcagaaacccctgccctttcccttcagaaaacgatggcaggcattcctttgagtttacaagcagagactcactccaacccaaactagctgggagttcagaaccatggtggaataaagaaatgtgcatctggt

SEQ ID NO: 535 - DNA, 191 bp, Homo sapiens; identical to SEQ ID NO: 115

SEQ ID NO: 536 - DNA, 261 bp, Homo sapiens
gaatggttactgtttatactgtggtatgtttttgattacagcagataatgctttcttttccagtcgtctttgagaataaaggaaaaaaaatcttcagatgcaatggttttgtgtagcatcttgtctatcatgttttgtaaatactggagaagctttgaccaatttgacttagagatggaatgtaactttgcttacaaaaattgctattaaactcctgcttaaggtgttctaattttctgtgagcacactaaaagcgaaaaa

SEQ ID NO: 537 - DNA, 217 bp, Homo sapiens
ttctttgtcaatctatggacatgcccatatatgaaggagatgggtgggtcaaaaagggatatcaaatgaagtgataggggtcacaatggggaaattgaagtggtgcataacattgccaaaatagtgtgccactagaaatggtgtaaaggctgtttttttttttttttttaaagaaaagttattaccatgtattttgtgaggcaggtttacaacacta

SEQ ID NO: 538 - DNA, 286 bp, Homo sapiens
acttcagggaagtttcataaaaggaagaattttaactcagcgtagaaagatgggtaaattttcctcatgtgaaggtgtactgcctggggtgctgcaggctggagatgagtattaaaataggaggagagtgagtgaaagcacaggaggaggaagggtcaggcaagtttggtgaacacagcaactggctatagaacaggagcagtgggagaaagtccagaaaggtctgctgagcttcagttgtacaaagctgtggagtttggcctttggtcatggctagattaggtct

SEQ ID NO: 539 - DNA, 191 bp, Homo sapiens
aatctctttttttctggaggctggcacctgattttgtatccccctgtagcagcattactgaaatacataggcttatatacaatgcttctttcctgtatattctcttgtctggctgcacccctttttcccgcccccagattgataagtaatgaaagtgcactgcagtgagggtcaaaggagagtcaacatat

SEQ ID NO: 540 - DNA, 267 bp, Homo sapiens; identical to SEQ ID NO: 123

SEQ ID NO: 541 - DNA, 211 bp, Homo sapiens
gtcaggtcttggtaggtgcctgcatctgtctgccttctggctgacaatcctggaaatctgttctccagaatccaggccaaaaagttcacagtcaaatggggaggggtattcttcatgcaggagaccccaggccctggaggctgcaacatacctcaatcctgtcccaggccggatcctcctgaagcccttttcgcagcactgctatcctcca

SEQ ID NO: 542 - DNA, 132 bp, Homo sapiens
aaaagttcacagtcaaatggggaggggtatttttcatgcaggagaccccaggccctggaggctgcaacatacctcaatcctgtcccaggccggatcctcctgaagcccttttcgcagcactgctatcctcca

SEQ ID NO: 543 - DNA, 157 bp, Homo sapiens; identical to SEQ ID NO: 119

SEQ ID NO: 544 - DNA, 258 bp, Homo sapiens
  misc_feature (61)..(61): n is a, c, g, or t
atggagtataagctgtttgtagtcaggcctggagtaatgagggtacctaaatactgaaggnatttttatgcagattgactgaaacctgaatcaaattggaaggagagggctgaattttgatagactggaagtattagagaattttctatactttgactcaaggaatggtcaacttttaggaaaagcaactatattatgtctgttaagatcatagaatcttaacctgaaagggaccttggagactatttagtacaactc

SEQ ID NO: 545 - DNA, 65 bp, Homo sapiens
ttaacagtacatttgtgtggctctcaaacatccctttggaagggattgtgtgtactatgtaatat

SEQ ID NO: 546 - DNA, 140 bp, Homo sapiens
  misc_feature (71)..(71): n is a, c, g, or t
  misc_feature (82)..(82): n is a, c, g, or t
  misc_feature (94)..(94): n is a, c, g, or t
cacagccaaaacctctacatccctacattcacacacttcctccacacaccatcctgaagtcaccccaactnctaccaccaanatcaccaccaancccaccagtataggaagcagcacacccatggcccacactacctcag

SEQ ID NO: 547 - DNA, 287 bp, Homo sapiens
agtgagactgactgcaagccccaccctccttgagactggagctggcgtctgcatacgagagacttggttgaacttggttggtccttgtctgcaccctcgacaagaccacactttgggacttgggagctggggctgaagttgctctgtacccatgaactcccagtttgcgaattatagagacaatctattttgttacttgcacttgttattcgaaccactgagagcgagatgggaagcatagatatctatatttttatttttactatgagggccttgtaataaatttc

SEQ ID NO: 548 - DNA, 213 bp, Homo sapiens
tgtgttggggtagactgctcctgcagagtttggaagaagtcaccagcaaagccggcctaaccaagaaaagtcaaggcccttcatgaccttgctgggcacagaaaacaccctcgtggagtacactaatttgaactggactggtctcagtgtgagcacttggcacactttactaaacacatatacaaccccaccgtgagtcaactttaaagtaaa

SEQ ID NO: 549 - DNA, 86 bp, Homo sapiens
actgagggggtcctggtgtgcatttgcaccctaaagctgcttacggtgaaaaggcaaataggtatagctattttgcaggcaccttt

SEQ ID NO: 550 - DNA, 62 bp, Homo sapiens
tatgtaattataagatgaagcgtagtgaattgtacagctgttgtaataatgacctatttcta

SEQ ID NO: 551 - DNA, 124 bp, Homo sapiens
ggctcaggtcctccctacaagacctaccactcacccatgcctatgccactccatctggacatttaatgaaactgagagacagaggcttgtttgctttgccctcttttcctggtcacccccactc

SEQ ID NO: 552 - DNA, 220 bp, Homo sapiens
ctgtatcactgccttcgtttatatttttttaactgtgataatccccacaggcacattaactgttgcacttttgaatgtccaaaatttatattttagaaataataaaaagaaagatacttacatgttcccaaaacaatggtgtggtgaatgtgtgagaaaaactaacttgatagggtctaccaatacaaaatgtattacgaatgcccctgttcatgttttt

SEQ ID NO: 553 - DNA, 255 bp, Homo sapiens
attattttgatagcagatgtgctatttatttatttaatatgtataaggagcctaaacaatagaaagctgtagagattgggtttcattgttaattggtttgggagcctcctatgtgtgacttatgacttctctgtgttctgtgtatttgtttgaattaatgacctgggatataaagctatgctagctttcaaacaggagatgcctttcagaaatttgtatattttgcagttgccagaccaataaaatacctggttg

SEQ ID NO: 554 - DNA, 179 bp, Homo sapiens; identical to SEQ ID NO: 134

SEQ ID NO: 555 - DNA, 290 bp, Homo sapiens
gaccaaatggattagactgtaaactgcaaagtgctgtccgcacatgaggtcatctgattactgtcctcagatctcttttgtagaggatttcaatgtatttctttatcatttgagtgtgtgtgtgatggacgaatatgtgtgtgagtttgagaagcatatcgttcgtgtccagttactttgcaaatttgtggacatttgtgattggacagaggggtttgtgctgtggcctaacacttgccaagtgaggtgtaggttatgcctatatgcaaattaaacttcacctttcttga

SEQ ID NO: 556 - DNA, 168 bp, Homo sapiens
aggagggaaaataatagcccagtgagagctgaatgaaaagggactgaatttaaatatttgtaagaactttgtgatgatgagtaattgtcagacgtgggatagataactgagaggctcagaatctttaccaaggatattttttaggataaggtagctgcctgttcatga

SEQ ID NO: 557 - DNA, 151 bp, Homo sapiens
tgtaatgctaaaactgaaatggtccgtgtttgcattgttaaaaatgatgtgtgaaatagaatgagtgctatggtgttgaaaactgcagtgtccgttatgagtgccaaaaatttgtcttgaaggcagctacactttgaagtggtctttgaat

SEQ ID NO: 558 - DNA, 217 bp, Homo sapiens
gaccccagtggaaaacaaagccaaacaaaactgaaccacaaaaaaaaggctggtgttcaccaaaaccaaacttgttcatttagataatttgaaaaagttccatagaaaaggcgtgcagtactaagggaacaatccatgtgattaatgttttcattatgttcatgtaagaagccccttatttttagccataattttgcatactgaaaatccaataatc

SEQ ID NO: 559 - DNA, 117 bp, Homo sapiens
ttaaacagtctagagtgaagggaatgttttaaaatccagaggcgatcaagtgaagccaacctttggaggcccgtagaagtcatttggaggaatttggacttcgtgcagtaggaaaga

SEQ ID NO: 560 - DNA, 127 bp, Homo sapiens
aagagttaaacagtctagagtgaagggaatgttttaaaatccagaggcgatcaagtgaagccaacctttggaggcccgtagaagtcatttggaggaatttggacttcgtgcagtaggaaagagggaa

SEQ ID NO: 561 - DNA, 155 bp, Homo sapiens
aaacattaccgggcatggtagcttgcacctgcagtcccagctacttgggagactgaggtgagaggatcacctgagtgtaggaggtgaaagcctcaccgaactatgactgaaccactgcactccagcgtgggcacttggcaccagagcaagattct

SEQ ID NO: 562 - DNA, 217 bp, Homo sapiens
gtgtgttaaaatgagggtctcactgctttaggattgaagtggctggaaagagtgatgcctggggaaggagatggagttatgagggtactgtggctggtactttctgtactaaacatttcctttttctattttaccactaattttgttttaaactgtgagccgtccaagtcagaagaagacagcaaaaaaagcaacttttccaacatacaatttactt

SEQ ID NO: 563 - DNA, 172 bp, Homo sapiens; identical to SEQ ID NO: 163

SEQ ID NO: 564 - DNA, 258 bp, Homo sapiens
atgaaaggtggaagttctacctagatttgaatgagtgtttttttaagggaatgagaatgtcatggtgctaaacctgacaaataagagatcattgaaatgctgaaaattttaacagtttttttaaaagtattgagggggcaaaaattaccaattatggtatacaaaaataagcctataaatgtgtttcacattgctaacttgagtttcagttgattcagtttgtaataactagtaatgagcttctgtttacaataaaaa

SEQ ID NO: 565 - DNA, 143 bp, Homo sapiens
cttgttcttttgaagcttgtgctgaggttttagcttttctatgttttatatgccgctgctttgaaagagaacctagattctatagttgtattattgttgtttcatactttaaatttatatggctgtggaaaaacgaattaaaa

SEQ ID NO: 566 - DNA, 227 bp, Homo sapiens
agcccaacttcttacccgaaagcatcactgccttggcccctccctcccggctgcccccatcacctctactgtctcctccctgggctaagcaggggagaagcgggctgggggtagcctggatgtgggccaagtccactgtcctccttggcggcaaaagcccattgaagaagaaccagcccagcctgccccctatcttgtcctggaatatttttggggttggaactcaa

SEQ ID NO: 567 - DNA, 32 bp, Homo sapiens
ttataatgagtgcgatatatgttgtcgaggct

SEQ ID NO: 568 - DNA, 252 bp, Homo sapiens
ggttctgtaaggtctttatttcccataagtaaatattgccatgggaggggggtggaggtggcaaggaaggggtgaagtgctagtatgcaagtgggcagcaattatttttgtgttaatcagcagtacaatttgatcgttggcatggttaaaaaatggaatataagattagctgttttgtattttgatgaccaattacgctgtattttaacacgatgtatgtctgtttttgtggtgctctagtggtaaataaat

SEQ ID NO: 569 - DNA, 139 bp, Homo sapiens
cccctccaagaccctgtgttcatttggtgttcctggaagcaggtgctacaacatgtgaggcattcggggaagctgcacatgtgccacacagtgacttggccccagacgcatagactgaggtataaagacaagtatgaat

SEQ ID NO: 570 - DNA, 245 bp, Homo sapiens; identical to SEQ ID NO: 262

SEQ ID NO: 571 - DNA, 97 bp, Homo sapiens
ttctccccaaccacttagtagcaacgctaccccagggggtaatgactgcacactgggcttcttttcagaatgaccctaacgagacacatttgcccaa

SEQ ID NO: 572 - DNA, 260 bp, Homo sapiens
gagtaggttcggtctgaaaggtgtggcctttatatttgatccacacacgttggtcttttaaccgtgctgagcagaaaacaaaacaggttaagaagagccgggtggcagctgacagaggaagccgctcaaataccttcacaataaatagtggcaatatatatatagtttaagaaggctctccatttggcatcgtttaatttatatgttatgttctaagcacagctctcttctcctattttcatcctgcaagcaactcaaaa

SEQ ID NO: 573 - DNA, 162 bp, Homo sapiens
tgtttttgacatcagctgtaatcattcctgtgctgtgttttttattacccttggtaggtattagacttgcccttttttaaaaaaaggtttttgcatcgtggaagcatttgacccagagtggaacgcgtggcctatgcaggtggattccttcaggtctttcct

SEQ ID NO: 574 - DNA, 257 bp, Homo sapiens; identical to SEQ ID NO: 173

SEQ ID NO: 575 - DNA, 193 bp, Homo sapiens
  misc_feature (120)..(125): n is a, c, g, or t
  misc_feature (127)..(127): n is a, c, g, or t
gaagaaagctaactcagtacactaagagtgatttacatgcctgcaaataatttgtgtctggggtcttgaccctccccaaatgccttgttatttatatctctgcttttagataacagatgnnnnnntntctatgggcttgtaccggcagaggcaacagcaggtccttaagactccccaggtgccatgatgaaaa

SEQ ID NO: 576 - DNA, 243 bp, Homo sapiens
gagtaaaaagaaccctttgctgagtaaccaagcctttaattttgtgtttttatgaaaggaattaaaatacccacgataaatatttaccacaacctgtgtcagataaatgggaaattaaacacagattgtacaatgtgagcttgggagttaatggcccagattttactgttaggcagtaagagttggagtaggtagtcttgttatcatgagaagaaccttgaacagatacaactaatttacata

SEQ ID NO: 577 - DNA, 74 bp, Homo sapiens
cgatatgacttccatgtaaacgttcatccactctgcctgcttacaccctgccctcatgctaatgtaataaactc

SEQ ID NO: 578 - DNA, 241 bp, Homo sapiens
ttcatcatatccaagttcactctgtcttcctgagcagtggaagatcatattgctgtaacttcttttaagtagttgatgtggaaaacattttaaagtgaatttgtcaaaatgctggttttgtgttttatccaacttttgtgcatatatataaagtatgtcatggcatggtttgcttaggagttcagagttccttcatcatcgaaatagtgattaagtgatcccagaacaaggaatactagag

SEQ ID NO: 579 - DNA, 241 bp, Homo sapiens
  misc_feature (93)..(93): n is a, c, g, or t
  misc_feature (97)..(97): n is a, c, g, or t
taagaatttatgaaactgggcaagttatttcctgggactcaatggtaaagacacagcagtaatccaaattttccatctttgaaattttccatncttncagtcaatattagtaatacctgggtcaaaggggagagttaggcataccaattaatgatcatcagaaatgacatagtcctacaaaagcaaagaaaatttagagacactttcttaaaaatacgactcttggtactgttgaagaaaa

SEQ ID NO: 580 - DNA, 57 bp, Homo sapiens
tattgaggacccatggtaaaatgcaaatagatccggtgtctaaatgcattcatattt

SEQ ID NO: 581 - DNA, 162 bp, Homo sapiens
gcattctcaagaggtcgtgccaatcagccactgaaaggaaaggcatcactatggactttctctattttaaaatggtaacaatcagaggaactataagaacacctttagaaataaaaatactgggatcaaactggcctgcaaaaccatagtcagttaattctt

SEQ ID NO: 582 - DNA, 269 bp, Homo sapiens
atttgcattttaccatgggtcctcaataaataaatagaatgttgttttttgtattttaagtttttttttgtttttcccctcagaggaaggatgaaaaaaagaattaactgactatggttttgcaggccagtttgatcccagtatttttatttctaaaggggttcttatagttcctctgattgttaccattttaaaatagagaaagtccatagtgatgcctttcctttcagtggctgattggcacgacctcttgagaatgcatgcatgaa

SEQ ID NO: 583 - DNA, 163 bp, Homo sapiens
agaattaactgactatggttttgcaggccagtttgatcccagtatttttatttctaaaggggttcttatagttcctctgattgttaccattttaaaatagagaaagtccatagtgatgcctttcctttcagtggctgattggcacgacctcttgagaatgcat

SEQ ID NO: 584 - DNA, 56 bp, Homo sapiens
cacaatctgagcacgctaccaaatctcaaaatatcctaagactaacaaaggcagct

SEQ ID NO: 585 - DNA, 263 bp, Homo sapiens
gagatcacttggtaactggtttcatgtgtatccaaaaatcagcatttggatttaagctttctgaatttggtagtttaagaaacagatttagtttttcagtggttttaactcatgtgaaataatgattttccaccagctttgatgcaaagagatataattttaatgaacgatttatccagcagtttgttccaggggttgcctctccttatttacggggattactttgtacatgcagataagttttcgcaaacctatttccattt

SEQ ID NO: 586 - DNA, 223 bp, Homo sapiens
ccatgtcagcctggatagaggtatatgacgtgtgccaagaatttatcccagactcccctgtgtgacagcttcataataaagttacttaactgtgcctcttcctccttcctctccccacacaggatggatgggcatctttctccttgaccaccctactctcccttcctcccctgatcacctcccctccctgctctcccctggtgatggacttctaacatgagat

SEQ ID NO: 587 - DNA, 210 bp, Homo sapiens
tttcaagatcttttgctcacaattcactgcaactgaggggatgtgaatatcattatgcaataaattaagagccacagttggctgaggtgatatgaaagccaacctgcctaaggggggtatgaaagatgtgtatctttccaaacttttaaaacaacgtaagtctgagataagaacatatttgatggcactgtttggaaagaggtgtcctta

SEQ ID NO: 588 - DNA, 176 bp, Homo sapiens
tcctgcaggcatccgtgggggaaaaaaaatctctcagaaccctcaactattctgttccacacccaatgctgctccaccctcccccagacacagcccaagtccctccgcggctggagcgaagccttctgcagcaggaactctggacccctgggcctcatcacagcaatatttaacaa

SEQ ID NO: 589 - DNA, 242 bp, Homo sapiens
aggaagtacccgctccataagacccttacatttggacagtcaaggtgcacaattgtatgtgaccacaaccatgcaccttggacataaatgtgtgtaactgcacatggcccatcccatctgaataaggtcctactctcagaccccttttgcagtacagtaggtgtgctgataaccaaggcccctcttcctggcctgttaacgtatgtgattatatttgtctgggttccagtgtataagacatg

SEQ ID NO: 590 - DNA, 237 bp, Homo sapiens
aataaatgcctcaggcgtgctttttgattcatttgataaacaaagcatcttttatgtggaatataccattttgggtcctgaggataagagagatgagggcattagatcactgacagctgaagatagaagaacatctttggtttgattgtttaaataatatttcaatgcctattctttgcaaggtactatgtttcgtaaattaaataggtctggcccagaagacccactcaattgcct

SEQ ID NO: 591 - DNA, 243 bp, Homo sapiens
ggcagtagatagtcaaagtcaaatcatttctaatgttttaaaaatgtgctggtcattttctttgaaattgacttaactattttcctttgaagagtctgtagcacagaaacagtaaaaaatttaacttcatgacctaatgtaaaaaagagtgtttgaaggtttacacaggtccaggccttgctttgttaccattctgatgttggactaattgactaatcacctacttatcagacaggaaacttg

SEQ ID NO: 592 - DNA, 225 bp, Homo sapiens
cctccaccaatcatactttgacatttatctatttccttctccacttatggatgtaattggcttgctatagaaactacagttcagatgctttgaatgtatgaactacaatgaacaataaagtcctcttcttttgaagcatattttggcttcagctttaagataatcttatgacaagaagggtcacactgattcacttaataaattccattcttacctaacacaagg

SEQ ID NO: 593 - DNA, 220 bp, Homo sapiens
ctaattgattagaactgagtcttttatatcaagctaatatctagcttttatatcaagctaatatcttgacttctcagcatcatagaagggggtactgatttcctaaagtctttcttgaatttctattatgcaaaattgccctgaggccgggtgtggtggctcacacctgtaatcccagcactttgggaggctgaggtgggaagatcccttactgccagga

SEQ ID NO: 594 - DNA, 131 bp, Homo sapiens
atacctgttaaatagatcaattttgattgcctactatgtgaactcactgttaaaggcactgaaaatttatcatatttcatttagccacagccaaaaataacgcaatacctatgttagcattttgtgaactc

SEQ ID NO: 595 - DNA, 189 bp, Homo sapiens
ttaccctccaaaagcaagtagccaaagccgttgccaaaccccacccataaatcaatgggccctttatttatgacgactttatttattctaatatgattttatagtatttatatatattgggtcgtctgcttcccttgtatttttcttcctttttttgtaatattgaaaacgacgatataattattataa

SEQ ID NO: 596 - DNA, 178 bp, Homo sapiens
tactgatctttatattacagatttttttttcttttaggattagttcagcttgccccccctttccatttccaccatttatagtgagcctctccataattagtgccaaccattagtttcgttcatatttttacaccaggagtcaacaaactgtggccattggccaaatatggcctcccaa

SEQ ID NO: 597 - DNA, 47 bp, Homo sapiens
aggataaacttgtgtggtgtagagaagttaaaatcctcacgttgtac

SEQ ID NO: 598 - DNA, 213 bp, Homo sapiens
tacaaagaatatttgggcccagtgctacagaaaaacatgaactacatcttatcgtcacaaaatagccattataaaatgaattttgcagcctctgtttttttgaactttgaaataaaatgttcagacaaatattcaactttttaaaaacctccattcattgatagcctgagaaatgtacaatgaacatgtttaggcagactgctagtattttgc

SEQ ID NO: 599 - DNA, 80 bp, Homo sapiens
agagccaggacagtgaggtcaactttgacaattccatccacccagaagtcttggagctgctgcttgactatgcgtactcc

SEQ ID NO: 600 - DNA, 223 bp, Homo sapiens
tcatgttaaagagccgtgtctcccgccagcactcctcaccccggtatgaatgtgtttcctccacattgtatatccttccaccctctggctgcctagatcagtaaataaaattgatgtaatataatttataagtaacactgttgaaaccctgatcccagtggaggctgtaacccacctgcccccgcaccacccccctgacccctgttaccgcatttgtgtgtat

SEQ ID NO: 601 - DNA, 175 bp, Homo sapiens
tatttcttgctattgtgatatgacaagagacttaacttatcttgctctgttttcccctgtacacgctgtatataggggtcaatgtgatgctgctggagacgagaataaactggactagaatagtgcattgtatttagtctgtattgatcatggatgccctccttaatagccatat

SEQ ID NO: 602 - DNA, 163 bp, Homo sapiens
ctgcaaaagccgagatgggttccatgcagttctccagtgggacatcagtgcttatccgaatgtcatcaatggcaatctctccggaacgtcctttccctatcactccctcgaacacaatctggtactccatgtcgtagctgggcaggatgatccgcccgtgctt

SEQ ID NO: 603 - DNA, 238 bp, Homo sapiens
actggtatattattgcttcatgttttgtaccatcataagattttgtgcagattttttttacagaaattattattttttatgacaatatgacacttgtaaattgttgtttcaaaatgaacagcgaagccttaactttaaatgacatttgtattttcagacactgagtagcataaaaaccacatagaactgaactgtaacttaaattccaaactatgactactacattccaaagaaacag

SEQ ID NO: 604 - DNA, 134 bp, Homo sapiens
cagaggagctttattagagggacagggtgaaacatatttacaccggccgagcagggaccttaagaagcaggcgtgggagcagggtcccagctcagacgagttccaccttggcattggggtacaccgccaccacg

SEQ ID NO: 605 - DNA, 275 bp, Homo sapiens
  misc_feature (195)..(195): n is a, c, g, or t
ccacagtgttcccactaatgctattttttaattttttaatttagtttgtcataatttggtttcatcaactcctttgttttttccttctttttttttttttgagatgaggtctcactatcttgcccagactggtttcgaattgccctccagcaattctcccacctcagccttcagagtagctggcattgtgggtangcaccactgtgcccagctcctgttttataataaataagccagagctctatctccaaatggtgcaaatcatcaatgctatt

SEQ ID NO: 606 - DNA, 157 bp, Homo sapiens
aggtggttttgataacacacttataaggctttctgtaaaaggtactatagaagggcgaagaatcgttcaactgtcaatcagcctcttgattctttgtaaattgccagggtgggtgggtacatatctcttcttgattctgcatttcatacttaactat

SEQ ID NO: 607 - DNA, 282 bp, Homo sapiens
tcacctcctgctggctaccggggcaggcatgcacccggtgccagccccgctctgggcaccacctgccttccagcccctccaggacccggtccccctgctgcccctcacttcaggaggggcctggagcagggtgaggctggactttggggggctgtgagggaaatatactggggtccccagattttgctttaagggggccagaccctttgccaggctggattgtacgggccccaccttcgctgtgttcttgctgcaaagtctggtcaataaatcactgcactg

SEQ ID NO: 608 - DNA, 147 bp, Homo sapiens; identical to SEQ ID NO: 302

SEQ ID NO: 609 - DNA, 216 bp, Homo sapiens
gcaatatatttgtgattccccatgtaattcttcaatgttaaacagtgcagtcctctttcgaaagctaagatgaccatgcgccctttcctctgtacatatacccttaagaacgccccctccacacactgccccccagtatatgccgcattgtactgctgtgttatatgctatgtacatgtcagaaaccattagcattgcatgcaggtttcatattct

SEQ ID NO: 610 - DNA, 262 bp, Homo sapiens
gaggtgtgattttaaggcattgttatatttttttttatttgtgagtgttttaaaattttgtatttcttttaaactttttattttagaaaaatttccaacatatatagaagtagactattgtaaggaacccttatgtaccctccaccagcttcaacaactatcaacaaaagtttgatcttgttttaaccacattcctttccaatttttgtgtttacccccagattattttgaagcaaattcctgacctcataacattttcaaa

SEQ ID NO: 611 - DNA, 280 bp, Homo sapiens
acccacacttaaactaaaggctaagaatataggcttgatgggaaattgaaggtaggctgagtattgggaatccaaattgaattttgattctccttggcagtgaactactttgaagaagtggtcaatgggttgttgctgccatgagcatgtacaacctttggagctagaagctcctcaggaaagccagttctccaagtttttaacctgtggcactgaaaggaatgttgagttacctcttcatgttttagacagcaaaccctatccattaaagtacttgtta

SEQ ID NO: 612 - DNA, 258 bp, Homo sapiens
ggatgaagtggcacacactctaactgaaagcagagtattaaagaacactagacatccctttttaacatccttgaaatattccttccagacaaaagaccgtttgtgttttgtgatggaatatgttaatgggggcgagctgtttttccatttgtcgagagagcgggtgttctctgaggaccgcacacgtttctatggtgcagaaattgtctctgccttggactatctacattccggaaagattgtgtaccgtgatctcaa

SEQ ID NO: 613 - DNA, 140 bp, Homo sapiens
ttattattatgaaccttcagcctactttcttgagtgccgtaaaagtgcttgtaaattttttttttttttaagaagaaagaaaaaaatggtgtttgacgttgatggaaattcaaaaatatatatggaactgaaacattaac

SEQ ID NO: 614 - DNA, 156 bp, Homo sapiens
aatatacctgatgcgctgtagaatgaaaatgtaaaagataacctgtatgtgttccgagctttaattttttgtttacaaattgaacagtgttacatgggctgtccagtcctgattatagagaggaagaaatggtaacagtatggcagataagaatta

SEQ ID NO: 615 - DNA, 298 bp, Homo sapiens
agaggcccatgccaacagtctaatctaagagattagtctttcaaactcaccatccagttgcctgttacagaataactcttcttaactaaaaacctagtcaaacaaggaagctgtaggtgaggagatctgtataatattctaatttaagtaagtttgagtttagtcactgcaaatttgactgtgactttaatctaaattactatgtaaacaaaaagtagatagtttcactttttaaaaaatccattactgttttgcatttcaaaagttggattaaagggttgtaactgactacagcatg

SEQ ID NO: 616 - DNA, 267 bp, Homo sapiens
aggcattatatatactacacagagtacaattaaaccataattgggaattatattttgtttttttcttcccaggcaatacacctctgaacatgtgtgtgataaatgggtttgctaatgtgctgttttaaagtataaagcataatatgttttggttaacacaatgtactttttgaactataaatctttattttaatatggaaatgtttggaacaggagatgcaagccactaacagagaactttaataattctaccctgtattttataaa

SEQ ID NO: 617 - DNA, 187 bp, Homo sapiens
ttcagtttgtcctcatagggaatcaagtattttagctaggtgatgtcttgcaagtacgttccactttgttacaatctactatctgtatatactatttgtatcttaattcttttatgagatgttctgtaacatttttctcactttgacaaatgtttttagactgtacagtcaagatctggcgcttggg

SEQ ID NO: 618 - DNA, 212 bp, Homo sapiens
cctgagtcctgggatcagacaccccttcacgtgtatccccacacaaatgcaagctcaccaaggtcccctttcagtccccttccctacaccctgaccggccactgccgcacacccacccagagcacgccacccgccatgggagtgtgctcaggagtcgcgggcagcgtggacatctgtcccagagggggcagaatctccaatagaggactgag

SEQ ID NO: 619 - DNA, 183 bp, Homo sapiens
tcacctgaggcgttcaaaagatataaccaaataaacaagtcatccacaatcaaaatacaacattcaatacttccaggtgtgtcagacttgggatgggacgctgatataatagggtagaaagaagtaacacgaagaagtggtggaaatgtaaaatccaagtcatatggcagtgatcaattatta

SEQ ID NO: 620 - DNA, 48 bp, Homo sapiens
taggttgtcaacaggtactatttgtcacataactaactttcgaggcac

SEQ ID NO: 621 - DNA, 246 bp, Homo sapiens
cagggttcctttgcctgctaacaagcccacgtggaccagtttgaatgtctttcctttacacctatgtttttaagtagtcaaacttcaagaaacaatttaaacaagtttttgttgcatatgtgtttgtgaacttgtatttgtatttagtaggcttctatattgcatttaacttgtttttgtaactcctgatttttccttttcggatactattgatgaataaagaaattaaagtgatagttttattgg

SEQ ID NO: 622 - DNA, 158 bp, Homo sapiens
ttttcgtagtccaaaggctttattgttctgctgaaatgcttacaaatactgaaaacccccagcctgggcccaggcaaccaagggctcaatgctgggaaggagagcaggggaggtgggcttagtgttaaggcgtgaagggcgaggccagacagctggag

SEQ ID NO: 623 - DNA, 57 bp, Homo sapiens
atgaggcagaatctggttgggtatgtttcttatatatgtttgaagcagatggctgac

SEQ ID NO: 624 - DNA, 229 bp, Homo sapiens
gatggctgggagataagtccttgaatggaggaaccaagaggtgagttagaggcataaactaaggaatttcagttagtagggtttaggagtgacagtctagaatgagtggagactaggagattcatcttgatgcaagcatacttagatccatgttactcaggatagcataggtgagagaggagctggtagaattttaatgtcatacctgggtagtacaactggttattat

SEQ ID NO: 625 - DNA, 242 bp, Homo sapiens
actcaaggctgtgaacaaacatacgctgctttattctttccaatttttctcttgttttctagaactcttatccatatgtttttaaaataagtacctaaaagtggtttgatagtgtcctaaacgacttttttaacttcctaaatggaaagagcataacaatgtagttgattggtaagatttacagggatttggtttctgagtttgaggcacattcccagtgaataagctgagtcccataccac

SEQ ID NO: 626 - DNA, 156 bp, Homo sapiens
aactgatgggaaaggaccaattatttatagtttcccaacaaaagttttaagattttttacctttgcatcagtgcatttttatttatatcaaaaggtgctaaaatgattcaatttgcattttttgatcctgtagtgcctctatagaagtacccacag

SEQ ID NO: 627 - DNA, 121 bp, Homo sapiens
atataccataggctaaaactaaggctttcactctagaatgcaaagctgttttgcagctgttttcccttaaagatgtcctgttgctttagtgatatttagacccctctcagttaagaaatgc

SEQ ID NO: 628 - DNA, 234 bp, Homo sapiens
atggtaatgtaggatcctttcacagagtgccaggctaaagagctgaactttgtggtggaagagacagaccccctatgtgctctgtagacacctgtgatgaagtagaactcatgaggatatgaagagaaacatttgtaatttgagtgattaaactaggaacgaaagaggaggggagaaataggaagagagaatcaccggccctgttgactgatttgagctgggaatgaagaagaa

SEQ ID NO: 629 - DNA, 116 bp, Homo sapiens
gtgagtaaataatgttctagtgcaacaggacaaactactctctccacaggaaacccaaccacaacaggatcaatagaaagaaaagagaaaacgttagcccccaactacaaataaat

SEQ ID NO: 630 - DNA, 49 bp, Homo sapiens
tgattactagtgtaaactggttattgagatagattatgacattggtgga

SEQ ID NO: 631 - DNA, 87 bp, Homo sapiens
aaatgactttcaactaaccttgtgaatcttttgcagtgtactgtgtgcaataccaagggcatagctccctgtaatttgggaaataca

SEQ ID NO: 632 - DNA, 239 bp, Homo sapiens; identical to SEQ ID NO: 3

SEQ ID NO: 633 - DNA, 115 bp, Homo sapiens
gcatgctcagtcttttcctcttatctacaatacaaagggtttgtctgaaaagtctggttttttttctttttacaaatgtaccttagctgcatcaacaggagtaagatgtagaaaa

SEQ ID NO: 634 - DNA, 72 bp, Homo sapiens
gggagattggtggcccttgccaggaagtgtcttaacactttgtggatactgctgcctgttgtctttaaaagc

SEQ ID NO: 635 - DNA, 113 bp, Homo sapiens
gaaaactgttagatgcacactgttgattttcatggtggattcaagaactccctagtgaggagctgaacttgctcaatctaaggctgattgtcgtgttcctctttaaattgttt

SEQ ID NO: 636 - DNA, 41 bp, Homo sapiens
tcctcatggtggcagcgctcatagcgaaagcctactgtaat

SEQ ID NO: 637 - DNA, 275 bp, Homo sapiens
agcattaataatttccatgcatgtgtctttttccagtaggtatggttgaatttatgtaaatttattgctaatcccatcccttacgatttagagtataagctgcgcaagggcagaagtttttatttggtttgttcatggatgtattttaagagctgagaacagggcctggacacaataagcattcaataaatatttactgaatgaatgaactcctacctatattcctatttataatttggctccactttatcctactttagctcccattcaattca

SEQ ID NO: 638 - DNA, 157 bp, Homo sapiens
tgtgttttcagagctaggtacagaggaatgtttgctacctttagcggtgaaaaaagaaagagagtcaagaattttgttggattgtgtttgtgtgtgcatatatttgatatcatcattatatttgtaatctttggacttgtaatcatagcctgtttat

SEQ ID NO: 639 - DNA, 249 bp, Homo sapiens
tctccagagggcacttcggctgcctttgcttcctttcattcgaggcccggctcttgctgacagaataggttccgttttgggcggtggttctcgagcctgccattcaaaaccaaagcaaattggagcatttctcacaacatggtattgaagttcctttttgttctcaaaagttgtgaccgtgttaaattgtactcccttagtcctgtaaggtatgttaagtgaatcgcagttacgctgtacttttattaa

SEQ ID NO: 640 - DNA, 87 bp, Homo sapiens
gccctgcgccggcaaccacctagtggcccagggaaggccgataatttaaacagtctcccaccacctaccccaagagatactggttgt

SEQ ID NO: 641 - DNA, 128 bp, Homo sapiens
caggctgggccgtgagcaggtgggccgttgagttacctctgtgctggatcccgtgcccccacttgcctaccctctgtcctgccttgttattgtaagtgccttcaatactttgcattttgggataataa

SEQ ID NO: 642 - DNA, 274 bp, Homo sapiens
ggttgagtttgtccattgctagggagagacttccagtaataaaatttactattctagatgcttctactgttatgttttatctgcccatttatctttcttagttaccaggagaaatgtgtgacacctatattataatgaaaacaatcttattacttatagtttatctatattaaacaaatttaattgcattttaaagcattctttgatattgttgcttttgcaataaatatggataatcttggttataagggagttaaaacaatgctgtaataaa

SEQ ID NO: 643 - DNA, 247 bp, Homo sapiens
ggcaggcttctctgtagaaccccaggggcttcggcccagaccacagcgtcttgccctgagcctagagcagggagtcccgaacttctgcattcacagaccacctccacaattgttataaccaaaggcctcctgttctgttatttcacttaaatcaacatgctattttgttttcactcacttctgactttagcctcgtgctgagccgtgtatccatgcagtcatgttcacgtgctagttacgtttttct

SEQ ID NO: 644 - DNA, 263 bp, Homo sapiens
ttgttggtagagacggtgattcactatgttggccaggctagtcacgaactcctgacctcgtgatccgcccacctcggcctcccaaagtgctgggattacaggtgtgagccaccgtgcccggcctctttttatttattcctaaaatattaccttgaggccaaattctgcgcttaaggagaatgtgcaccaagtgctggggtgggggctggttataaacgaggccacaaatcatgcttgttaataaattgtgtggttcaaatctg

SEQ ID NO: 645 - DNA, 108 bp, Homo sapiens
gaggaggtgggcctcgttagactgctcagattactggaacctcgttatcacatcccattctacaagtttttcactgaatgtttcctgacatctataaatgagggtgcc

SEQ ID NO: 646 - DNA, 97 bp, Homo sapiens
ggccaaaaagtctacattgcgtgtgtggatggatgaatgagcagtgggagtgcagcgccaggtgacaagatgttgtgaggggttttgagtcatccag

SEQ ID NO: 647 - DNA, 220 bp, Homo sapiens
tgcccccagtatgtttaggacgcgagccccagaaggatttgggagtaaacttaacattcactgtgtttttgctttgcatccgccatttgtgtgtgtttttggactgtgggctgtgtgtaccttggttggtgactcagtgagaagaagcaggaatgccaaagatactatgaatgttttgagttttgttgctgttgttgttgagaggttgtttcactggtat

SEQ ID NO: 648 - DNA, 201 bp, Homo sapiens
aactctgtaaggaggaccatgtcagactattgtaagctaagcattaggactgatacaaataatatatgctcctggcatagaaaaataaaccacagagaacgagttcaaagaatagcaaagaaagaaagaggacccagtgggcgaaagatgagagtgtacttttaccaaaagttatctaagcctgagcacttgaagtctgca

SEQ ID NO: 649 - DNA, 205 bp, Homo sapiens
aatcccaattttcaggagtggtggtgtcaataaacgctctgtggccagtgtaaaagaaaatccctcgcagttgtggacatttctgttcctgtccagataccatttttcctagtatttctttgttatgtcccagaactgatgttttttttttaaggtactgaaaagaaatgaagttgatgtatgtcccaagttttgatgaaactgt

SEQ ID NO: 650 - DNA, 235 bp, Homo sapiens
gcagcttcctgataaagcgtgctgtgctgtgcagtaggaacacatcctatttattgtgatgttgtggttttattattttaaactttgttccatacacttgtataaatacatggatatttttatgtacagaagtatgttttttaaccagttcacttattgtactttggcaatttaaaagaaaatcagtaaaatattttgcttgtaaaatgcttaatatcgtgcctaggttatgtgg

SEQ ID NO: 651 - DNA, 163 bp, Homo sapiens
ctttcattggaagttcatcactgttaggcgttatcttgagtattataacaaagcaaatccacaagtattcaatataagattaggaaaaaaattcctgcgatactttgttgtcaaacacttgccactgatagacgttattttagcttttaaggcctgtcacatt

SEQ ID NO: 652 - DNA, 115 bp, Homo sapiens
taatattttttgctgcctaggcaaatggcttttgtgaaaacacttgtatgaaaagcaatacaccatttgtttttacttaccaatcactatcattaggttttgatgcaaatgggaa

SEQ ID NO: 653 - DNA, 210 bp, Homo sapiens
gttttgatgctgtgtgcttttggctgggcctcgggctccaggccctgggaccccttgccagggagacccccgaacctttgtgccaggacacctcctggtcccctgcacctttcctgttcggtttagacccccaaactggagggggcatggagaaccgtagagcgcaggaacgggtgggtaattttagagacaaaagccaattaaagtcca

SEQ ID NO: 654 - DNA, 99 bp, Homo sapiens
aattcatatcccctgttcgtctcatgcgcgtcctccgtccccaatctaaaaagcaattgaaaaggtctatgcaataaaggcagtcgcttcattcctctc

SEQ ID NO: 655 - DNA, 89 bp, Homo sapiens
tgtgcccagcctaagccatttcttaaaataaaaatgctaaaggactagtaagtaaaaataaaacttcctatgggatttcccagtggaaa

SEQ ID NO: 656 - DNA, 36 bp, Homo sapiens
ataatgaaagttagtaacgtccattatttaataaag

SEQ ID NO: 657 - DNA, 100 bp, Homo sapiens
ttttctaaccttgattgaccatgggcaaatgaaactgcagaaagtgaaactgcggataggggggatgactgtattcaatagattccgacattatgtctgc

SEQ ID NO: 658 - DNA, 259 bp, Homo sapiens
  misc_feature (139)..(139): n is a, c, g, or t
caaaaatgagtcacaactcttgattccatccacctccaaaatccaggtattttccttaattctcattctttcacaactccacatatatccatatcctcattatctcaggacaacgtgaccatttcttgccacttcccancacttccatgcctaccaaagaagcctatcttctctcaccaggaccactgaaaaagtcttgcaactgatttcccttgtcctcttcttgcctctctacagtcaattctctatacaacagtca

SEQ ID NO: 659 - DNA, 34 bp, Homo sapiens
tttatgaacagcagactctatgtaaaggcatttt

SEQ ID NO: 660 - DNA, 262 bp, Homo sapiens
ccaggttaggaaaggattcagcactacagcatacccctctacaacatacagccctgtcacattgagatcataatccctcctgtcccactcctctctaccaaccccaccctactagctaggtcttcagtgttttacattgaatattggtacattttaattattttttctcataaatgggttatttatagaaattttgttaactcttgagccatatgcatgtgtagatactggcagggctatgtttgtttatgatgctctgcaa

SEQ ID NO: 661 - DNA, 174 bp, Homo sapiens
ccatcagtttttccaatgtgaatggactggttcatatcacaccatatttagagatacaaggtgattataactaacgtgtctacaagacatactgggtcaaacaatgtgatcaatccaaagggtatctttttaaaaagaatttaagtactcagctgcaaagataagttcactaat

SEQ ID NO: 662 - DNA, 161 bp, Homo sapiens
attagtgaacttatctttgcagctgagtacttaaattctttttaaaaagataccctttggattgatcacattgtttgacccagtatgttttgtagacacgttagttataatcaccttgtatctctaaatatggtgtgatatgaaccagtccattcacattg

SEQ ID NO: 663 - DNA, 248 bp, Homo sapiens
atgccctgaggccagttggcgaggggtggctcctgagggtttttataccctttgtttgctaatgtttaattttgcatcataatttctacattgtccctgagtgtcagaactataatttattccatttctctctgtgtctgtgccaagaaacgcaggctctgggcctgccccttgcccaggaggccttgccagcctgtgtgcttgtgggaacaccttgtacctgagcttacaggtaccaataaagaggc

SEQ ID NO: 664 - DNA, 188 bp, Homo sapiens
tctgtgtgtgtcctagcatggagagttccttccttttcccttcagttaggttgaccctttccatttgtttagtatccgtgcacatgtcgtactagaccccaatcaagttgcttatttaaaattctttcagctgtttccctattatttccttactttgctgaacatgtccgctgttttacctcactgct

SEQ ID NO: 665 - DNA, 289 bp, Homo sapiens
tatacagaatagtcactggtcttgggctaaatggtgacttcaagtgtagtggctgcatagtcaaaaatgaattagatgagtacaaaagtgacgaaatgaaagaatgtcaagaatggaccacaaagacagtgttttatgacctaaagattaagatttatccatttgtgtacaattgtggactatataaaataaaacaagactttgacctcagtggataagaagtatttggatgtactaatcaatatttttggtctgggtcagtggtgggttcatctgtgtttgttgtatt

SEQ ID NO: 666 - DNA, 230 bp, Homo sapiens
aagccgaggggcccgagactctgcagcggggccagaaagagaagagtgggggaggaggccgggagtggtgcatggaccagggggtagagggaggtgggtgtggacctggggtcgggcgccagtcagcttgcagcctatgaaggacggaagggagggctacagagataggggaagagtggggctgaggatagccagagcggcttggcacacagttttagggtaaaagcatc

SEQ ID NO: 667 - DNA, 238 bp, Homo sapiens
agttggtgccctgattccatttttctcaagttgtagaagaaacaaactaatttacggtaggagaaattcaaattcagattctccccatccccaccagttacctttggttggtggagagggggagaattggcaggaaaggggcacaaagaaactttttggggtgatggaaatattttgtaccttgctttagatgttggtcagatggaatatacgtttgtggaaacctgccaaactgtac

SEQ ID NO: 668 - DNA, 188 bp, Homo sapiens
tattcctggtagaggaatttctgtatttgaaaattctccagaaggaataatataaactgtggactttgggtgataatgatatgtaggttcgtcagttgttaacaaatgtatccctctgttgggggctattgataatggggaaggctgtgcatgtgtgggagtaggaggtgtatgggacatctctgtac

SEQ ID NO: 669 - DNA, 189 bp, Homo sapiens
tcaagttaatggcagctaaaacgctcccagtccatttattggccacatgaggtggtcgtcaagaaacaagttagaaggttatgacaggaagtagtataataaatgcccggcagtacgaggggttcaacagaagtgaacaaggcacaagaaagaggtctgtgttcaggaaacaggccagtccccacatgg

SEQ ID NO: 670 - DNA, 298 bp, Homo sapiens
agcaggagtaagtttctcatcccatgggcgaccagggccatctcctcccaccagtggcccccactcacagggagctggcaatgccctacctgcctgttctccagatggagaaacaggctctgagatttcacaggtcttgcccaaagtcattgattttgatgattaaaaagaataaacacagtgtttcctgagtagcagtgattgttatgccttgctattttaataaagattctattttcgtataacattgtcaagtggaaacatgctgaaatctattaaaccatctttgtttgtggaa

SEQ ID NO: 671 - DNA, 269 bp, Homo sapiens
cttaggatatgaaatcttcagaggatggtagaagagaaaggtaaatgccataagaaagtagagtacttgtggtcattgaaaccatggaatttactcagggatagtgtatatagtgagaaatcaggtaactaagatttgagcctaaacgtaatctgtaaaaggggagctcaaaggaaaagggaatgggaggaccggattttgtaaggatagtagggcaaatgttaagagagagaatgagagagttgagtatggcaaagagtgactcaatt

SEQ ID NO: 672 - DNA, 277 bp, Homo sapiens
tggaggattcaagatgagcaggtatgttttctgctttcaataactcattttctgctgcagagatagccttgtaagcaagcaaaggaaaactttgacatttctctgcaaagataatgcattacatataagggtgtgtctgggagggtaccaggtgcctgtcagcaaaagttgcaaaaacagcttgataagggtattaagtgggcctgttgggaaaggcaggagtgtcaaatgtcggacagaactccagacagagaaatccagatatccagtaggttag

SEQ ID NO: 673 - DNA, 246 bp, Homo sapiens
tgaactaaaactatgagccttattcaatatctataattctatgatttttttaaattatgggaaattaatgaaagatgtttacatgaataatgtttgcccttactgtgttatgaatgagttttttgtagtgtgtctgggtgcatgatgcaagagagtaggaaaaatgtttttgaaacaaaacttgacaaatatttgtaatgaaagtaaatttaaagattgctataattgcgctatagaaacaatgca

SEQ ID NO: 674 - DNA, 236 bp, Homo sapiens
attggagtggatgggttctgccttaaattgggaggactccaagccgggaaggaaaattcccttttccaacctgtatcaatttttacaacttttttcctgaaagcagtttagtccatactttgcactgacatactttttccttctgtgctaaggtaaggtatccaccctcgatgcaatccaccttgtgttttcttagggtggaatgtgatgttcagcagcaaacttgcaacagactg

SEQ ID NO: 675 - DNA, 177 bp, Homo sapiens
gataacagtcttgcatgtttatcatgttacaatttaatattccatcctgcccaacccttcctttcccatcctcaaaaaagggccattttatgatgcattgcacaccctctggggaaattgatctttaaattttgagacagtataaggaaaatctggttggtgtcttacaagtgagct

SEQ ID NO: 676 - DNA, 213 bp, Homo sapiens
tattttccggttgaccccagaattcattagatttttttaaaaaacaatttcaaaatagttgctgttttaaattagttgcatccagttcatatcaatgtttgcatgctttttagtctttgttatttattgaaaacctttggtacctaaacttaagtttgattgtttcagtgtgtacttggtaaatatgtcagtggccttttaactaaacatcaa

SEQ ID NO: 677 - DNA, 235 bp, Homo sapiens
ttctacactagtgccatgggaaccaggtctgaaaaagtagagagaagtgaaagtagagtctgggaagtagctgcctataactgagactagacggaaaaggaatactcgtgtattttaagatatgaatgtgactcaagactcgaggccgatacgaggctgtgattctgcctttggatggatgttgctgtacacagatgctacagacttgtactaacacaccgtaatttggcatttg

SEQ ID NO: 678 - DNA, 52 bp, Homo sapiens
agtgcaacgtattcaagtcctcaatatcctgatcataataccatgctatagg

SEQ ID NO: 679 - DNA, 218 bp, Homo sapiens
gtacatggtctggagtaacctttatatgaagtggctgcccaggtgtgtggcttaggactagatctccaggttgcacaaagttggcattgggtttagtttgcatttttccattctgaagatggccctccttggatttcatccaggaaatccatagctttctgttaacaggacatggagtagactggctgcatttgaaggacagcacagatccctcatca

SEQ ID NO: 680 - DNA, 131 bp, Homo sapiens
gtaatttgtgattgtcgaggaagaggtgtggctgttggtgtgatagtaatactgctggtgactttattggttgttttgtttagtgccccgttaattaagccttgagttcggttatcctgcagtggtgctga

SEQ ID NO: 681 - DNA, 120 bp, Homo sapiens
tacaatgatggttgagtgaaaatacagaaggggggtttgagtattcagatttcataaaacacttccttggaatatagctgcattaacttggaaagaagcctgttgggccagaagacagaa

SEQ ID NO: 682 - DNA, 179 bp, Homo sapiens
tcatggaatgctacatgctttctgtttttttcattttggatttctccaaaactaactgaatttaagcttcaggtccctttgtatgcagtagaaaggaattattaaaaacaccaccaaagaaaataaatatatcctacttgaaatttactttatggacttacccactgctagaataaatg

SEQ ID NO: 683 - DNA, 294 bp, Homo sapiens
tgggaagcggcgagatcctcgggctgggggtgcccacgtttgctacctccccctgtgaaatcgctggtgctcacaattgtctttcacagtgtatgtgatttttttaaggaaaaaaaaaaaatccctatttaagattttgaaggtgctaccattattttgccacagactttgaagaaacttttggatgtggggcatcatccgcatctttctctctcctccaaatgacaaagtttggggaatttttgaattttcctagcatcgcccttgtgctcatcaggtaatctgctaaggagg

SEQ ID NO: 684 - DNA, 277 bp, Homo sapiens
tacccccttttatatctaatgtagaaaaagcgaaattgaatctggaaagcaaactgttgtatatagttgcggtaacaatcatgaagagagagccgggctgtcccctcagtaattcattttaaataacaaattatttaaaaataaaattcatgccagagccagctgaagaggccttccttcatcaccactgaggccacccccaatctgggccctctgtccatctggcatgtctcctcccagcaagattcatctgttcaatgccatttgcgtttcaata

SEQ ID NO: 685 - DNA, 235 bp, Homo sapiens
  misc_feature (157)..(157): n is a, c, g, or t
aggatttcccaaaccagccgaggccccagcacccgccgtctccccagaagccccctcctccttcccccatgggtcatatgttgaaagtctattttaaaaactatgttccttgccgtagattgcagagctaatttatcacgtttctctcctgtgaganccccccttttatatgatatatccagaggaagttttgtaatataaaacaggacgcccacactgatggttttgcactggt

SEQ ID NO: 686 - DNA, 193 bp, Homo sapiens
agagggaactgtgcagtccccaggccgccccggctccgggctagaggcaataaataaacccgatcctgccgggcacagccgcgcccgcgcctccggcgccgtccccgggctgacgggggagggagcggagaagcgagcgcagattctgcgtataaatcagctctggagcagacacagcccggctgtgaaaagc

SEQ ID NO: 687 - DNA, 178 bp, Homo sapiens
  misc_feature (84)..(84): n is a, c, g, or t
  misc_feature (89)..(89): n is a, c, g, or t
  misc_feature (123)..(123): n is a, c, g, or t
gagacaaggacagacaaattttctggctgtccccatttctcctgggggaggggtttggggctggtttgactttaattggtgggngggtngtttctgccgctctgtttgctgcagtccccgtgncctgcttggggactgagaaatttgagccaggtatccagagccacagcccatcttg

SEQ ID NO: 688 - DNA, 252 bp, Homo sapiens
agttcggcaaaggtgtcctagaaatggccagagtttttgacagacagagtgtagtctccttattagaagaaaggaaaaaaaagcagaggccaaagaagttttgtgtttgctgatgagagccccactcatttgcgaaacgcacgtaaaacaaagtgaaccgtgactgttaaactagggatgggaaattttgcatcttggggggctgtacatttatttatttagttgaagattcactgatcccactttgaaata

SEQ ID NO: 689 - DNA, 174 bp, Homo sapiens
caccccaggactttatcaacttgttcaagttctgaatcccagcacatgacaacacttcagaagggtccccctgctgactggagagctgggaatatggcatttggacacttcatttgtaaatagtgtacattttaaacattggctcgaaacttcagagataagtcatggagagga

SEQ ID NO: 690 - DNA, 293 bp, Homo sapiens
ttgcttttcattcagatgtatacataaacttatttaaaatgtcatttaagtgaaccattccaaggcataataaaacccgaggtagcaaatgaaaattaaagcatttattttggtagttcttcaataatgatgcgagaaactgaattccatccagtagaagcatctccttttgggtaatctgaacaagtgccaacccagatagcaacatccactaatccagcaccaattccttcacaaagtccttccacagaagaagtgcgatgaatattaattgttgaattcatttcagggct

SEQ ID NO: 691 - DNA, 168 bp, Homo sapiens
catttcatgtttggcgggcatgtgagtgcacaagatggaaagagcgattggagcatcctggtataattacccccattgtgcttttaatggaaatttcaaaggacgggagtattttgttggttggtgtccaggtttgtggcactgttccaagaggccttacacacacac

SEQ ID NO: 692 - DNA, 243 bp, Homo sapiens
acaaaagagccagagttctggacccatgtttggagcatttgtagccttatttttttgcgtgtgaatcttttaccctgaaaaaaagccataatgaattaagccagactgaccacttgcttggagtgtgtgcttgaaaaaaccagagcaatactgttgggtattgtatcaggcttcagtacaaactggtaacaccaatgtggatcctgacagctttcagttttagcaaaaatacacgtgaaatct

SEQ ID NO: 693 - DNA, 207 bp, Homo sapiens
gagtatgagtatgcctgagtgtgaatgtgtatgagtgtgaatgagtgtgtgcacatgagtgcacgggtgtcagcatgtgtatataagtgtgggcatgtgtatgtgattgtgtgagtgtgggcaggtgagtgtgttggggatgtgggttagggtggggagtggtgctttctctagtgtgtcctccggaacatcttgcctacctagcaa

SEQ ID NO: 694 - DNA, 105 bp, Homo sapiens
gtatttattgagtcacggattattgtgcatcaagcaattgttaatatgacctggtcctatggggtagaacttaggaaaaataaagttggttcttattcaatattt

SEQ ID NO: 695 - DNA, 231 bp, Homo sapiens
  misc_feature (92)..(92): n is a, c, g, or t
  misc_feature (105)..(105): n is a, c, g, or t
  misc_feature (160)..(160): n is a, c, g, or t
  misc_feature (169)..(170): n is a, c, g, or t
  misc_feature (172)..(176): n is a, c, g, or t
  misc_feature (178)..(180): n is a, c, g, or t
  misc_feature (182)..(182): n is a, c, g, or t
  misc_feature (186)..(187): n is a, c, g, or t
gaatggcaaaacacaaatggcaacataaatgtccatttgacttacctaacttcacaactttcaagttgaggatgtcatttattcttgaattntgtttttttactnagatgctttcaattaatagccctatatttttgtgcaggcgaactgtataacaggnataaaaaannannnnnannnantgannaggaggagaaattctcacagaacaccatatgagctttagaccaa

SEQ ID NO: 696 - DNA, 208 bp, Homo sapiens
ctctgagttttatatgctggaatccaatgcagagttggtttgggactgtgatcaagacaccttttattaataaagaagagacacaggtgtagatatgtatatacaaaaagatgtacggtctggccaaaccaccttcccagcctttatgcaaaaaaaggggagaatcaaagctttcatttcagaaatgttgcgtggaaaagtatctgta

SEQ ID NO: 697 - DNA, 35 bp, Homo sapiens
ttggatagcagctatcttgttggatgtgaggtgga

SEQ ID NO: 698 - DNA, 237 bp, Homo sapiens
cagacacatgagcaggactttggggagtgtgttttatatctgtcagatgcctagaacagcacctgaaatatgggactcaatcattttagtccccttctttctataagtgtgtgtgtgcggatatgtgtgctagatgttcttgctgtgttaggaggtgataaacatttgtccatgttatataggtggaaagggtcagactactaaattgtgaagacatcatctgtctgcatttattga

SEQ ID NO: 699 - DNA, 151 bp, Homo sapiens
gcagaaatatgtaaccttagactcagccagtttcctctgcagctgctaaaactacatgtggccagctccattcttccacactgcgtactacatttcctgcctttttctttcagtgtttttctaagactaaataaatagcaaactttcacct

SEQ ID NO: 700 - DNA, 91 bp, Homo sapiens
tgtacagggtcattttgacagtaactggtatattcctttgcattttatgttgcattgccaatttttagtgtatccagtttgaaagtataat

SEQ ID NO: 701 - DNA, 269 bp, Homo sapiens
agacctagaaggttgtagatgggaaatcaggaatgatttgaactgataaagatttcagactcataagaacacattttataaatgttaaacacaaaaactacatgactgaagatagaagagaatgcgatggattttattacacatggtggaagagagaagaggcgtgtaggtttgcaaacaaagttaagaaataggaaactgaatttttcattgtacagaaaatgtatctcttggggagggcctgtgtacccccattctctgattataaa

SEQ ID NO: 702 - DNA, 223 bp, Homo sapiens
tacattgtaagagcaacatcctacgttaataaatgttctagcccggtgcttcgcgaacatttatgtgcatacaaatcacctgggatcttgttagaaggcagtaggtctggggtggggcctgagattctgcatttctaaccaggtcctgggagatgctgatgctatcgagccacaaccacactttgagtagcaagcctctggcctatccttattgtttgttata

SEQ ID NO: 703 - DNA, 258 bp, Homo sapiens
cattctttcctccacaggattgctttgtccatctcctgctttcatttcaagtgcataaacaaaacctcaaagggcctgggaaggtgaggcaggccagagtctgtgttctgtgttgagtgtcaagctatttgttaagaaggtttgcaacaggcctttggtgtgggctttgccagagactgttttgaacactttgcttgagatccgtgccctgtaaaatggatatgatgttttactgatgtctgtaatacatttgtaaac

SEQ ID NO: 704 - DNA, 85 bp, Homo sapiens
ttaagcgcattagaaaacaattgtttgtaatggaatcaaagtgtttccctggacagtttgatgtgcttatggttgagatttataa

SEQ ID NO: 705 - DNA, 33 bp, Homo sapiens
gtgagcccatatcgtactgctgcagtccagcct

SEQ ID NO: 706 - DNA, 135 bp, Homo sapiens
gatatcagatcagttgagactaacagttgaaagcagtaaacatattacggacatattattgataaaagacatttatgaagaggataaactgtgaaggtgtacagacactaaaccatagttgctaaacacatacaa

SEQ ID NO: 707 - DNA, 208 bp, Homo sapiens
gaggccccagaggacttatctcagctgtacatgctctgcctgtggagacatggcttttctttgtgctgtggcagactggggctttggaagtggtgtatgtttaacttacctgagagtgagagatgtgtaggaagaatagctggaagaaagtgaaagatgagtgccagtacttttggcctgttatccagtagagagaaagtgacagtga

SEQ ID NO: 708 - DNA, 103 bp, Homo sapiens
attaaagtactcaagttagttgttttgcagagatgttgccttcagatgttaatcaggtctctcaagtttcatggagtctatgctgatcctttaattgacaaat

SEQ ID NO: 709 - DNA, 223 bp, Homo sapiens
gcatttgaattgactaggcttttcctatataaaaaactcaaaacttgttaactctgtactttaataaaatttaaaattaaaactgtgttgtttttttctcttctgctagatacatatataattaaagtactcaagttagttgttttgcagagatgttgccttcagatgttaatcaggtctctcaagtttcatggagtctatgctgatcctttaattgacaaat

SEQ ID NO: 710 - DNA, 281 bp, Homo sapiens
gggcccctggtatttatagggtccaagaggaggcacctgcttttcaactgcaccctcagtgctgcctcttcacggcccctaaacgtttccctttgaggttgtgatgctgggaatcacagacttcactctctgcctgcacccttccccgaggtctcatcttttctgggtcccacatctttgtaataatgtgaaaaagcacaatttgtctgatcaccccccaggtggttccccaccttattatcactacctgatccgagttactgcaataagtacggtgtcgc

SEQ ID NO: 711 - DNA, 130 bp, Homo sapiens
gggacaaggtcccttggtgctgatggcctgaaggggcctgagctgtgggcagatgcagttttctgtgggcttggggaacctctcacgttgctgtgtcctggtgagcagcccgaccaataaacctgctttt

SEQ ID NO: 712 - DNA, 55 bp, Homo sapiens
cagatcgacacgcagctagcctcctgcattgtatggttataaatagcaccctagt

SEQ ID NO: 713 - DNA, 168 bp, Homo sapiens
gtttgagctatcgaagagtaagtaaccagaaatttgagaacagcctgggcagcatagtgagaccccatctttacaaaaacttcacagattagccaggtatggtggcattttcctgtagtcccagctactcagaatgctgaggcaggagagtggcttgtgaccaagagt

SEQ ID NO: 714 - DNA, 255 bp, Homo sapiens
tggagatagctcttaaagatataaatgtttatggctgaaatgttatggcatcttggatttgctttaaaataacccagcttgctgcaggaggtgggtattgtgtgtgtgggaaggtggggaggctgcgggaggaagagatgacccaagattaggcagatgttgttaactgtggaagcagggtggtgagtgggggctcatgacattatgctctctactttgtgtacgtgtgaacatttccgtaataaaagatgcctt

SEQ ID NO: 715 - DNA, 83 bp, Homo sapiens
taaccttaggaaaccagaatagcgtttggcagacacgacgttttcagtttacctttgacacctgccccactccattttgcttt

SEQ ID NO: 716 - DNA, 52 bp, Homo sapiens
gaggccggtgccagtgcaggtccttggtgtgctgtgtgccggtcccctgggc

SEQ ID NO: 717 - DNA, 238 bp, Homo sapiens
agaattccagataaacacacagcctttcccatacctttttttttcttactataaaatattataagatccattgatgtccaaataataccaccgagcatctcttcacctctcctcctcttggtccacttgctaatgcccagttttcttctccatttccactttttcttaggctccctatttactattcattttgacttccttctgttttatttttttccctttagcattgcatgtgaat

SEQ ID NO: 718 - DNA, 220 bp, Homo sapiens
tctggtagataaagccctagaccttgtccacactctcacccccatccccaactttccctggaccggtgccgcccccacttgatgcttgctcaaggccggggactggagcgggctacttgtatatttcgttgtcagtctgcagaatgtgtttgatttttatttttccctccttctctgacatgtgtcaaggaataaagactggatacaggtccattacgtc

SEQ ID NO: 719 - DNA, 234 bp, Homo sapiens
ctgtggcatgctcagaggttcctgctggattccagctggagcggtgtgatacccttctttttcagctgttcgtgccttcctttcttgtatccaccaaagtggagacaaatacatgatttcaaagatacacagtacctacttaattccagctgatgggagaccaaagaatttgcaagtggatggtttggtatcactgtaaataaaaagagggcctgggaattcttgcgattccat

SEQ ID NO: 720 - DNA, 225 bp, Homo sapiens
gagtaccgcgcagacattaaaagtcatgtaaaagaacatttgactgaaagaaaaatgctccttgaatattaaaaggttgtaaaaatagtgcatgttatgtgatttcaattttgttttttaaaatatgggtgtatgcttgtatacgtagagcagataaaaaagacggaaggcatactaaaaaatgttgagtggttatctttgtatggtggaacaaagtcactgtaa

SEQ ID NO: 721 - DNA, 173 bp, Homo sapiens
aaatgctccttgaatattaaaaggttgtaaaaatagtgcatgttatgtgatttcaattttgttttttaaaatatgggtgtatgcttgtatacgtagagcagataaaaaagacggaaggcatactaaaaaatgttgagtggttatctttgtatggtggaacaaagtcactgtaa

SEQ ID NO: 722 - DNA, 266 bp, Homo sapiens
aaatgtttgacattcattatcagaatgagggaaaatttaaacaattccgttttttcttttgctagtaggcatattatgctaataaaattactaaattaaaagtgtgtcaaggatttgacaaactgccatttttctccagaagtcaagcccctaagtgattgtctagaggcaagaattttttgatatgttgtctcaacaatgcttctcacttcgtcttcaggtgccccaacccgcaagtacacatactatgtactcacttgaaaatg

SEQ ID NO: 723 - DNA, 151 bp, Homo sapiens
gtgtgtcaaggatttgacaaactgccatttttctccagaagtcaagcccctaagtgattgtctagaggcaagaattttttgatatgttgtctcaacaatgcttctcacttcgtcttcaggtgccccaacccgcaagtacacatactatgta

SEQ ID NO: 724 - DNA, 166 bp, Homo sapiens
  misc_feature (101)..(101): n is a, c, g, or t
agagaggtttgtcccatatatcttgttccagcagccatatatcttgtggtctacagcctgaagcatgatttcccttgaagtcttggggttgtttaaagganagtcccttcaatataaaacctctgaaatattagtgagaatggctcactaatgtgaacaatgttta

SEQ ID NO: 725 - DNA, 141 bp, Homo sapiens
atttttgcctttccgtttctgtctatgatgtaggcttctgaggagaaccaagaagcttggctttagtggtagaatgacagaacttagggatcccttgcaggctagaacaaagttctgacccttagaccaaatctttatgtt

SEQ ID NO: 726 - DNA, 299 bp, Homo sapiens
  misc_feature (127)..(127): n is a, c, g, or t
  misc_feature (194)..(194): n is a, c, g, or t
atttgcaacagcctagtggatttggtagggtcctgaatcatctctataaggcaaacaaggaaattgtaacacaaagaaataaacatattgaatataattgctattctgtaagacatacagtctgtgnaagatgtatcttatttacagagacatttttgaaaattaaaatattaaatactttttgttatataganacaatgatctggaagtataaaaagaaaaatattatcttgttgatgtaaatatgatatccttatatatattagaatccaataagatatcatgggcgcaatattagc

SEQ ID NO: 727 - DNA, 139 bp, Homo sapiens
aaaaagagagaatatgtcctgtgttagcttgcttaggaaataaagagagagagagagagggagggagggaaagagagaaagagagagagagaaagagagtgagagaaagagagagcaagagagcaagagtaagaaagaa

SEQ ID NO: 728 - DNA, 172 bp, Homo sapiens
aacacccataatcaatcacagagataaccactgttcataattccttccagtcttcttacttggcacatatacatttgtctttctttatatatgacatatggatattttacaaagttaggatcctactctatgcactgcttggtgatcggatctattcaatgtacaaaatatt

SEQ ID NO: 729 - DNA, 205 bp, Homo sapiens
cctttctaaggatgagggaatccacaacagactttctctagaaaacactaatgatggacaactttttggtgtcatcaatgagttggctactaccttgatgtaaaaatttgtaaggaaaattttcaccatttcgagtgtcaagtgtatttttaactgtctggtttgtacttttatgacttttgtactaccaaagcggagttaaaaa

SEQ ID NO: 730 - DNA, 98 bp, Homo sapiens
tggatcgatgatgagtccccccaaaaaaacattccttggaaaagctgaacaaaatgagtgaaaactcataccgtcgttctcagcggaactgaggtcca

SEQ ID NO: 731 - DNA, 97 bp, Homo sapiens
tggatcgatgatgagtcctccaaaaaaacattccttggaaaagctgaacaaaatgagtgagaactcataccgtcgttctcatcggaactgaggtcca

SEQ ID NO: 732 - DNA, 94 bp, Homo sapiens
tggatcgatgatgacctcaatacatgcattccttggaaagctgaacaaaatgagtgaaaactctataccgtcgtcctcgtcaaactgaggtcca

SEQ ID NO: 733 - DNA, 194 bp, Homo sapiens
tcaaactcaggtatgtgacactctacagttcaatgctagcacacctgtgtgaggcttaacaacatgaggaactgatagccagtgatacacaaatccagcacttcctctccatttactctgtcaggctgtatatggggagcaacacatatggctttgtggcagccagaaagtgaaggtctttttaggaggtgaca

SEQ ID NO: 734 - DNA, 290 bp, Homo sapiens
ttgacagtctgccttactatgaaattctaggtacaaaaattttccttaagctctgaagatgttgatccattgacttctggcattcagtgttgctgatgacaaatctgttagcagtctatttctcatccatttttgtgttgagctaatcgtgatgacctcatttgttttctccctggccactttaatatcttccttctatccttaatattccaaaattttacaatattgtgtctagatgtagattgtattgggccctctcaatctggagacttagcttgctctgttcttca

SEQ ID NO: 735 - DNA, 258 bp, Homo sapiens
gatcgtctggtaactttctaactttaaataatatgtttgagcaataatttcttgacttactgactttacaacatctttaataattccccattacaaaagataaggatttaacttacactatcgccactttcctttgtccatctctctccaaatgtctgatagttacatcactttttaatacatctattggtttgattttatagctttgaacaatacactaatcctctagttcttgttccattaactgaagatcttttc

SEQ ID NO: 736 - DNA, 269 bp, Homo sapiens
gcctggcctgattcagggccttgtggcccccagcttctgtttcaagctgggcagaccccaggatcccttccctccctaaggactcagctgaggggcccctctgcccccttctacctccacctcagcaccctcccccagcttgatgtttgggtctccccagcaccctcctccctggccggtgcaaagtacagggaggtaaagcaggacccttgcagacatgttgcccagcacacagtaggccctcaataaaagccatttgcactttaaat

SEQ ID NO: 737 - DNA, 264 bp, Homo sapiens
tttcttctttatgtccaattttgtgggtgggaggaggatttagtctcttcctgatttcgaagagctcatttactatttcgggaaataagatttggattgtcaaccattatagctattttttacacacttttcaactttgtttttgttataagaatgtgtatgattgttacatgtccaagtataaccatgttcgcttttatggcttttgagtttcatgtcatttttggaaagatatatatattgagtccataaaacccttcacct

SEQ ID NO: 738 - DNA, 46 bp, Homo sapiens
gacaggggttctaattactgtctgtgagagttactactttgtaact

SEQ ID NO: 739 - DNA, 129 bp, Homo sapiens
cgtccccaacatgcatctcactctgggtgtcttggtcttttattttttgtaagtgtcatttgtataactctaaacgcccatgatagtagcttcaaactggaaatagcgaaataaaataactcagtctgc

SEQ ID NO: 740 - DNA, 237 bp, Homo sapiens
ttattttatttcgctatttccagtttgaagctactatcatgggcgtttagagttatacaaatgacacttacaaaaaataaaagaccaagacacccagagtgagatgcatgttggggacgggggaggctggcagcaggggggccccggcggctcaccccagggctcccggagggggctgtttccatccaccacccaaaaaaacaccacaagggtcagtcctagcccacccgacagctt

SEQ ID NO: 741 - DNA, 92 bp, Homo sapiens
cctgcctgggaaagcgagtccacagttcactaacaaacacaataccatccacaaacaagtagccacaaagaccacagttagcaaacacacac

SEQ ID NO: 742 - DNA, 278 bp, Homo sapiens
  misc_feature (214)..(214): n is a, c, g, or t
gctggtctcgggcaacaagcagcctcctaggagcagaaggtgatggagggccacgggggcagggaggagcagatggccatgtggctcagcccctgcctgggaaagcgagtccacagttcactaacaaacacaataccatccacaaacaagtagccacaaagaccacagttagcaaacacacacagtcacacacacacacacacacacacacatnacgggaggtgggcaggaccgcagtctgcagtggggaggcaagtgttagttgcatcatcaggtgg

SEQ ID NO: 743 - DNA, 32 bp, Homo sapiens
atggacaattctgtaccccaataatcagaaca

SEQ ID NO: 744 - DNA, 164 bp, Homo sapiens
caggggggcctgagagtaacagtgaggattatccaggtgccctgagtgtaacagtgaggattatccaggggggccctgagtgtaacagcgaggattatccagggggccctgagtgcaacagtgaggattatccaggggggcctgagtgtaacagtgaggattat

SEQ ID NO: 745 - DNA, 286 bp, Homo sapiens
gtcttcaccaactaagaaagctctgttattctggcctgggtgcttgctccagactcaggaacatctggtcaacacaagcatcactgggctggggaattgtgtgtgtgctgcatcatctccgactctcttgtagttccttccttccctccctcactcttacatgcagacacagacagacacagtctggttgggacatgcagtggcagctcctggtgtataacatctttcacacaccttgagtctatctgcttgctgcctttgactgatcctgaaatggttggccttt

SEQ ID NO: 746 - DNA, 211 bp, Homo sapiens
gtttgtgtgggaactttgcaagtcagtttccctgtatgaagtgatggagagagtgattaagatactgagctttctctgtgttcttgccgttaaccattgccggtttgtgggagattaagaagtcgatgcgttttatggagaattaatttattttgatatagacagatggacgggtcatgaaaatttgttgacatactttactaaactgcta

SEQ ID NO: 747 - DNA, 113 bp, Homo sapiens
tgtcactggtgaaagacgacttggtgcccaatttttaataaacacaatgctattagcgtcactccaatttagtgtctgattgttaaatgttaatgtactgcactctacagttt

SEQ ID NO: 748 - DNA, 251 bp, Homo sapiens
gaaccgcagattatggttaatccaattctgtgcacctgaggtccataaataaaagaataagtattgaaatgaaagaatgacagaaagaatgaatggacacatgaacgactgaattagaaatggaaatgcctggcacagccaggaaggagctgcccatgggattgtcattcatttcactctgggcacctgaggtccataagcgtgaaaagaggcaggaagagaagtgtcagggagtcaaagatagagctaag

SEQ ID NO: 749 - DNA, 135 bp, Homo sapiens
gggcccagctcagaaccgggcagacacccccttcaaatgtcttcgcacgtaggttttgcacagtgtttatttgctggtgtctcagggatttgacagtttccttaatattcccacacatggccgagaaaaataaat

SEQ ID NO: 750 - DNA, 187 bp, Homo sapiens
ttctttatagtgttatgcttattttcaattttttttttcctgattctgtctggtacttagaattgtagtgtcttcatcatcaattaaagaaaactgtctaaatgaattcatggatgtaaatattagtggtccttaatgtctttgattgctggacatgaaacaaactgccaattaaattttgcggaga

SEQ ID NO: 751 - DNA, 66 bp, Homo sapiens
gtcaccccagagtcattgtcacctcagagtcattgtcactccagagtcattgtcacctcagagtca

SEQ ID NO: 752 - DNA, 258 bp, Homo sapiens
ctcaccaggactcgtctctccattcccgtcagagtttgctttgatttccctttcctttccttctcggggaccagttcttacttccttttatttttagctttgcactccatgtggtttcagggttcagtttgatccatcaaaaggttctttttttataatcccttttgaaaatgataatcaaaggaagagatgtggtgtttggtcatgtggaaaactcaatgtataatttagacgtctgtcaaaaatccgacaaataaa

SEQ ID NO: 753 - DNA, 119 bp, Homo sapiens
  misc_feature (91)..(91): n is a, c, g, or t
aaaatagagagacatacctggctccaaaacaaggctgtatcttctgccactgtaataaaatagatgcaattgaggttcataaataaaagantaaatacttaaacgtgaaaggtgactaa

SEQ ID NO: 754 - DNA, 218 bp, Homo sapiens
gtttcaattgattcacaactttaaaaaatatccacagaggcgtaagaggagatattgtattgcacccacgaaccagttttatgctgtttaagaaagggacattcaagaaacaaaaagggagctttgggaaatttgaacaataaataatagtagaataaaaaattcagagaaagtttggatgataaatttgagccaatctcccagtaagcagagcaaaa

SEQ ID NO: 755 - DNA, 209 bp, Homo sapiens
cagagtcattcattgtcaccccagagtcattgtcacccgagtcattgtcagctcagagtcattgtcaccccagagtcattgtcaccccagagtcattgtcaccccattgtcaccccagagtcattgtcacctcagagtcattgtcactccagagtcattgtcacctcagagtcaatgtcaccccagagtcatcgtcaccccagagacat

SEQ ID NO: 756 - DNA, 123 bp, Homo sapiens
catttaaataagaggagatccacaaagagcaacagagataagagaagaaaggaaataatcaataaaataattcaagaaaatccaaggacatgagttttcagattgtaagagactacttgagtg

SEQ ID NO: 757 - DNA, 278 bp, Homo sapiens
aaatggcttccgatttccagcttgggcctggggattggagatgtccccactgagagtagggcacaagtgaggaaatggtttggagaggaagatgataagttacatcatggatgtgctgagtctgagttgcctatgggacttggaatggggggtggcaaaaggtgtgtgatcttgagcaagatattcaactcttctgggccttggtcttctcatttgtaaaacggtgataagaatattacttcccatttgtgttgctgtgaatattaaatgcgctacca

SEQ ID NO: 758 - DNA, 123 bp, Homo sapiens; identical to SEQ ID NO: 756

SEQ ID NO: 759 - DNA, 278 bp, Homo sapiens
ctatcaggtactgtgctcatctgaacaacaaacctcaacaacacgcaatttatccatgtaattaacctgcacatgtacccccgaaacctaaaataaaagttcaaaaaaaacctgtggtatttaaataggtattgtgtctaaaaatgcatgctatctaaaaatgtagttttattgcactgtataagaatacgagaggtttaaaatagacactctaaaagttataagccctaatttacatatattctctagcctttctccaccttctatctaccaaaaaa

SEQ ID NO: 760 - DNA, 119 bp, Homo sapiens
attttctatctgtggttggattcagaccacatgaacactgagggctgactgtagttttgaatgtctgttactgaggaggcaccagcataaagtattttatcacttcagacgctgacaat

SEQ ID NO: 761 - DNA, 103 bp, Homo sapiens
gtttgtcttgcgcagtgcttactccagctgtggcatgcaggtgtcagcaagtatgatcagcaatgaggcggtggtcaatatcctgtcgagctcatcaccacag

SEQ ID NO: 762 - DNA, 196 bp, Homo sapiens
tttatttataccaagcctcctcctgaaatttctcttcctttctttctacctacccaagacataccacgtgcttcaataaccagtcccttcctcctacaaacactacaacctggaaagcactcttgcttttctgaagtcctctatacttagtgtaactcttctgtgatgaagattaaagtgtattatggcaactctc

SEQ ID NO: 763 - DNA, 115 bp, Homo sapiens
ttcattgaggttcgtgtgtgctgtgttcgcgtgtgtgtgctgtgtgtgcatgtgtgtgctgtgtcttagtgtgtgtgctgtgtgctagtgtgtgtgctgtggggtaccgagctcg

SEQ ID NO: 764 - DNA, 47 bp, Homo sapiens
gggatgcataaagtatgagtgccttttaggatgggaattgagatgta

SEQ ID NO: 765 - DNA, 66 bp, Homo sapiens
gtttttattaaaacacaaacgcacacacacacaaaattagccaggcatggtggtccatccctgtaa

SEQ ID NO: 766 - DNA, 143 bp, Homo sapiens
gtgtgagccactgctggctaatttttgtattttcagtagagacggggcttcaccatattggccaggctggtcttgaactccttccctcgctcccccaaaaatttgaatttttttttcaacacttttacacctgttatggaaaa

SEQ ID NO: 767 - DNA, 104 bp, Homo sapiens
tgctgcggtcgcctctctcaggctcccccgctcccccgggcctccgcttctcagccgggtgctgtgcgtttgagtgtgtgctgctgctcgctgtgtgtgctgtg

SEQ ID NO: 768 - DNA, 242 bp, Homo sapiens
aaagagtcaactttccggcggcgggggagaaatgataaaagagagagaggagggcagatcaccacctagaagcacatcctttgttcgcagaggggggagaaaaagtcggagagaaagaatgaaagaggggaaaaaaggcagttcggcacccggagaaaggaggcaattcggggagaggaggaggagaagaagaaaacacacacgcgcgcacgcacacacacaccgcggagagaaaagaacag

SEQ ID NO: 769 - DNA, 274 bp, Homo sapiens
tgctcagtgtcatgctgtgtgtgcatgtgtgtgctgtgtgttttgtgtgtgtgctgtgttcatgtgtgctgtgcatgcgtgtgtgctgtgtgtgcctgtgtgtgcggtgtgctgtgtccttgtgtgtgctgtgtgtgcgtgtgctgtgtgcatgtgtgtgctgtgttatgtcgtgtgtgcagtgtgtgctgtgtccgatgtgtgctgtgtacacatgagagagcagagtgtacatgtgtgtgctgagtctatcagaagatgtgtgtagctgcgg

SEQ ID NO: 770 - DNA, 267 bp, Homo sapiens
atgactgtgctagagccaccctctcactttagcctcctgagtagctgggactacaggtgcttcccactgtgcctggccaattaacaatttcatttttatttttagtagagatgagatctcactatgttgcccaggctggtcctgaactcctgagctcaagagatcctcccaccttggcctcccaaagtactgggattacaaacaagagccactgtgcctgaccaggctctaagattgctaatctggctatagaaggactaatgttag

SEQ ID NO: 771 - DNA, 98 bp, Homo sapiens
aaagagtcaactttccggcggcgggggagaaatgataaaagagagagaggagggcagatcaccacctagaagcacatcctttgttcgcagagggggga

SEQ ID NO: 772 - DNA, 127 bp, Homo sapiens
ttttttagttgccaacagttgtatttttgctgattatttatgaccttaaataatatatttttttttttaagaagacattttgttacataaggaaaacttttttattcaatggaataaattatggcat

SEQ ID NO: 773 - DNA, 114 bp, Homo sapiens
ttttttagttgccaacagtttatgtttgctgattatttatgacctgaaattatatatttttttttttaagaagacattttgttacataaggatgacttttttatacaaaggaaa

SEQ ID NO: 774 - DNA, 168 bp, Homo sapiens
aagaaaaggagaagcactacaagcttttgaaaaatggaaagagaaaaagatggaatatcttaaagagaaaaatagaaaggagagagaatatgaaagagcaaagaaacagaaagaggaggaaactgttgccgagaaaaagaaagataatttaactgctgttgagaaatg

SEQ ID NO: 775 - DNA, 133 bp, Homo sapiens
aatgcagaatattcctctgaacacttccctcatacatcattcaatattataaattaaacacaaagagcctctccacttagatttttatcatgcatcctacattgtaatgtctttactcttccatagaaaaggt

SEQ ID NO: 776 - DNA, 116 bp, Homo sapiens
ctgggccttggtccccagaagatggcggctagggcctcgccgccaggacagagaagggacggggtggctgggcagtcagggaaggagggtcgcccggatccgacattttggagaga

SEQ ID NO: 777 - DNA, 96 bp, Homo sapiens
ggatcgatgatgagtcccccataaaaacattccttggaaaagctgaacaaaatgagtgagaactcataccgtcgttctcatcagaactgaggtcca

SEQ ID NO: 778 - DNA, 96 bp, Homo sapiens
ggatcgatgatgagtcccccataaaaacattccttggaaaagctgaacaaaatgagtgagaactcataccgtcgttctcatcggaactgaggtcca

SEQ ID NO: 779 - DNA, 64 bp, Homo sapiens
ccatgaaggagcaagttttgtatttgtgacctcagctttgggaataaaggatcttttgaaggcc

SEQ ID NO: 780 - DNA, 101 bp, Homo sapiens
tgagtgtgctgtgctcgcgtgtgtgctgtgttcatgcgtgtgctgtgtgttgtgtgtgtgtagctgcggggatgcataaagtatgagtgctttttaggatg

SEQ ID NO: 781 - DNA, 106 bp, Homo sapiens
gtgcatgtgtgtgctgtgtctttgtgtgtgtgctgtgtgctagtgtgtgtgctgtgtgtgcatgtgtgtgcgtgtgctgtgcgtttgtgtgctgtgtgctcgtgtg

SEQ ID NO: 782 - DNA, 115 bp, Homo sapiens
gatgggaattgagatgtaagatttgggggtgagggtcgtgccaattacatttcatttgcatggattttggttttcatgctctgtcctcccctcctttggtcttactgggtccctc

SEQ ID NO: 783 - DNA, 217 bp, Homo sapiens
agcgaagggaaggttgcgggagaaagagcgggggccggggccagacgccaagaggggcgcggggagcacagagaagcggagggaagggcgccacgtcgaggggccgggggaggcggtgactggggggcggagtggaggctgcacccggaccgcgggcgcccagctcggtttgggccgacggagccctctgccgtcgcgagcccgggcctcgggaggg

SEQ ID NO: 784 - DNA, 158 bp, Homo sapiens
gttcaaatgatctacacttacattttgcaaatctttttttttaaattttttaaattttatattttttttccagccaactcaaggccaaaaaaaatttcttaatatagttattatgcgaggggaggggaagcaaaggagcacaggtagtccacagaata

SEQ ID NO: 785 - DNA, 74 bp, Homo sapiens
ggtgcaattattatactgcgaaaatgaaaatattgcatactaaacagtacctagggtatgatctcaatgtaaaa

SEQ ID NO: 786 - DNA, 150 bp, Homo sapiens
agggacccagtaagaccaaaggaggggaggacagagcatgaaaaccaaaatccatgcaaatgaaatgtaattggcacgaccctcacccccaaatcttacatctcaattcccatcctaaaaagcactcatactttatgcatccccgcagct

SEQ ID NO: 787 - DNA, 86 bp, Homo sapiens
atcatttcatagtcatttatgtttcatcggtcctcatgtgtactagtgcgttattttacttatactcccggatatcatattattta

SEQ ID NO: 788 - DNA, 183 bp, Homo sapiens
acaaaactctccaataatgactctgctcagctcaggggcagcaggagtggagtgtcgggggcccttggtgctgagtgaagataaattaaaaatcccaacaagccagtgacattatgtacaggaggaaaggggtggggcttccaggacagaggccgagggtggcagggcaggacttggagtggc

SEQ ID NO: 789 - protein, 349 aa, Homo sapiens
METPPVNTIGEKDTSQPQQEWEKNLRENLDSVIQIRQQPRDPPTETLELEVSPDPASQILEHTQGAEKLVAELEGDSHKSHGSTSQMPEALQASDLWYCPDGSFVKKIVIRGHGLDKPKLGSCCRVLALGFPFGSGPPEGWTELTMGVGPWREETWGELIEKCLESMCQGEEAELQLPGHSGPPVRLTLASFTQGRDSWELETSEKEALAREERARGTELFRAGNPEGAARCYGRALRLLLTLPPPGPPERTVLHANLAACQLLLGQPQLAAQSCDRVLEREPGHLKALYRRGVAQAALGNLEKATADLKKVLAIDPKNRAAQEELGKVVIQGKNQDAGLAQGLRKMFG

SEQ ID NO: 790 - protein, 349 aa, Homo sapiens
METPPVNTIGEKDTSQPQQEWEKNLRENLDSVIQIRQQPRDPPTETLELEVSPDPASQILEHTQGAEKLVAELEGDSHKSHGSTSQMPEALQASDLWYCPDGSFVKKIVIRGHGLDKPKLGSCCRVLALGFPFGSGPPEGWTELTMGVGPWREETWGELIEKCLESMCQGEEAELQLPGHTGPPVGLTLASFTQGRDSWELETSEKEALAREERARGTELFRAGNPEGAARCYGRALRLLLTLPPPGPPERTVLHANLAACQLLLGQPQLAAQSCDRVLEREPGHLKALYRRGVAQAALGNLEKATADLKKVLAIDPKNRAAQEELGKVVIQGKNQDAGLAQGLRKMFG

SEQ ID NO: 791 - protein, 23 aa, Homo sapiens
IRQQPRDPPTETLELEVSPDPAS

SEQ ID NO: 792 - protein, 24 aa, Homo sapiens
QIRQQPRDPPTETLELEVSPDPAS

SEQ ID NO: 793 - protein, 199 aa, Homo sapiens
METPPVNTIGEKDTSQPQQEWEKNLRENLDSVIQIRQQPRDPPTETLELEVSPDPASQILEHTQGAEKLVAELEGDSHKSHGSTSQMPEALQASDLWYCPDGSFVKKIVIRGHGLDKPKLGSCCRVLALGFPFGSGPPEGWTELTMGVGPWREETWGELIEKCLESMCQGEEAELQLPGHTGPPVGLTLASFTQGRDSW

SEQ ID NO: 794 - protein, 150 aa, Homo sapiens
METPPVNTIGEKDTSQPQQEWEKNLRENLDSVIQIRQQPRDPPTETLELEVSPDPASQILEHTQGAEKLVAELEGDSHKSHGSTSQMPEALQASDLWYCPDGSFVKKIVIRGHGLDKPKLGSCCRVLALGFPFGSGPPEGWTELTMGVGP

SEQ ID NO: 795 - protein, 85 aa, Homo sapiens
METPPVNTIGEKDTSQPQQEWEKNLRENLDSVIQIRQQPRDPPTETLELEVSPDPASQILEHTQGAEKLVAELEGDSHKSHGSTS

SEQ ID NO: 796 - protein, 57 aa, Homo sapiens
METPPVNTIGEKDTSQPQQEWEKNLRENLDSVIQIRQQPRDPPTETLELEVSPDPAS

SEQ ID NO: 797 - protein, 47 aa, Homo sapiens
METPPVNTIGEKDTSQPQQEWEKNLRENLDSVIQIRQQPRDPPTETL

SEQ ID NO: 798 - protein, 18 aa, Homo sapiens
QQPRDPPTETLELEVSPD

SEQ ID NO: 799 - protein, 21 aa, Homo sapiens
QIRQQPRDPPTETLELEVSPD

SEQ ID NO: 800 - protein, 18 aa, Homo sapiens
QIRQQPRDPPTETLELEV

SEQ ID NO: 801 - protein, 15 aa, Homo sapiens
QIRQQPRDPPTETLE

SEQ ID NO: 802 - protein, 12 aa, Homo sapiens
QIRQQPRDPPTE

SEQ ID NO: 803 - protein, 21 aa, Homo sapiens
QQPRDPPTETLELEVSPDPAS

SEQ ID NO: 804 - protein, 18 aa, Homo sapiens
RDPPTETLELEVSPDPAS

SEQ ID NO: 805 - protein, 15 aa, Homo sapiens
PTETLELEVSPDPAS

SEQ ID NO: 806 - protein, 12 aa, Homo sapiens
TLELEVSPDPAS

SEQ ID NO: 807 - protein, 19 aa, Homo sapiens
RQQPRDPPTETLELEVSPD

SEQ ID NO: 808 - protein, 18 aa, Homo sapiens
RQQPRDPPTETLELEVSP

SEQ ID NO: 809 - protein, 17 aa, Homo sapiens
RQQPRDPPTETLELEVS

SEQ ID NO: 810 - protein, 16 aa, Homo sapiens
PRDPPTETLELEVSPD

SEQ ID NO: 811 - protein, 15 aa, Homo sapiens
RDPPTETLELEVSPD

(19) United States
(12) Patent Application Publication
(10) Pub. No.: US 2019/0127805 A1
(43) Pub. Date: May 2, 2019

(54) GENE SIGNATURES FOR CANCER DETECTION AND TREATMENT

(71) Applicant: ALMAC DIAGNOSTICS LIMITED, Craigavon (GB)

(72) Inventors: Aya EL-HELALI, Craigavon (GB); Niamh MCGIVERN, Craigavon (GB); Andrena MCCAVIGAN, Derryadd (GB); Bethanie PRICE, Craigavon (GB); Laura KNIGHT, Craigavon (GB); Nuala MCCABE, Craigavon (GB); Richard KENNEDY, Belfast (GB); Paul HARKIN, Craigavon (GB); Charlie GOURLEY, Craigavon (GB)

(73) Assignee: ALMAC DIAGNOSTICS LIMITED, Craigavon (GB)

(21) Appl. No.: 16/084,928

(22) PCT Filed: Mar. 13, 2017
(86) PCT No.: PCT/GB2017/050712

§ 371 Date: Sep. 13, 2018

(30) Foreign Application Priority Data
Mar. 15, 2016 (GB) 1604398.6
Jun. 7, 2016 (GB) 1609944.2
Dec. 15, 2016 (GB) 1621384.5

(51) Int. Cl. C12Q 1/6886; G16H 50/20; G16H 50/30

(52) U.S. Cl. CPC C12Q 1/6886; G16H 50/20; C12Q 2600/158; C12Q 2600/106; C12Q 2600/118; G16H 50/30

(57) ABSTRACT

A molecular subgroup of cancer is characterised by misregulation of the MAPK signalling pathway and the epithelial-mesenchymal transition (EMT) pathway. Biomarker signatures can be used to identify cancers within the molecular subgroup. The signatures are also useful for identifying the treatment that is best suited for a given patient. A method for selecting a treatment for a subject having a cancer, comprises measuring the expression level(s) of at least biomarker selected from Table A or Table B in a sample from the subject. By assessing the expression level(s) of the at least 1 biomarker it can be determined whether the sample from the subject is positive or negative for a biomarker signature comprising the at least 1 biomarker. Based on the outcome of this assessment different treatments selected from MAPK pathway 10 inhibitors, EMT pathway inhibitors, SRC pathway inhibitors, taxanes and anti-angiogenic therapeutic agents may be indicated. Related treatment methods and products are also provided.

### FIELD OF THE INVENTION

The present invention relates to methods for selecting a treatment for a subject with cancer, predicting responsiveness of a subject with cancer to therapeutic agents including MAPK, EMT and SRC pathway inhibitors and determining clinical prognosis based on assessing the expression level of biomarkers.

### BACKGROUND OF THE INVENTION

Mitogen-activated protein kinases (MAPKs) are components of a vital intracellular pathway which controls a vast array of physiologic processes. These enzymes are regulated by a characteristic phosphor-relay system, in which a series of three protein kinases phosphorylate and activate one another. The extracellular signal-regulated kinases (ERKs) function in the control of cell division. The c-Jun amino-terminal kinases (JNKs) are critical regulators of transcription. The p38 MAPKs are activated by inflammatory cytokines and environmental stresses. Because of its role in cell proliferation and carcinogenesis, the most characterised MAPK pathway is the RAS/RAF/MEK/ERK pathway. Mutations of each of these genes are mutually exclusive, but all lead to constitutive activation of the MAPK signal transduction pathway. However this is one of the most frequently dysregulated signal transduction pathways in human cancers, often through gain-of-function mutations of RAS and RAF family members. Mutations in KRAS have been found in 90% of pancreatic cancers, 20% of non-small cell lung cancers (NSCLC), and up to 50% of colorectal and thyroid cancers (Jose et al., 1984) whereas mutations of BRAF have been identified in more than 60% of melanoma and 40% to 60% of papillary thyroid cancers (Cohen et al., 2003; Davies et al., 2002, Xu et al., 2003). Although MEK1/2 is rarely mutated, constitutively active MEK has been found in more than 30% of primary tumour cell lines tested (Hoshino et al., 1999). With regards to ovarian cancer, low-grade serous ovarian carcinoma which accounts for a small proportion of all ovarian serous carcinomas (<20%) are characterized by mutations of the KRAS, BRAF, ERBB2 genes (Lopez et al., 2013). In addition, it is hypothesised that alterations, other than mutation, exist in High grade serous ovarian cancer (HGSOC). It has been found that 11% of HGSOC have KRAS amplification and 12% have BRAF amplification, suggesting that the RAS/RAF/MEK/ERK pathway may have utility outside low grade serous ovarian cancer (The Cancer Genome Atlas (TCGA, Nature 2011). In addition 12% of these tumours have also alteration in the NF1 gene, a RAS GTPase and negative regulator of RAS (TOGA, Nature 2011).

The RAS/RAF/MEK/ERK pathway is activated by a wide array of growth factors and cytokines acting through receptor tyrosine kinases such as EGF, IGF, and TGF. The activated receptors recruit nucleotide exchange proteins which activate RAS through a conversion from the inactive GDP-bound form to the active GTP-bound form. Activated RAS recruits RAF kinase to the membrane, where it is activated by multiple phosphorylation events and where it activates MEK1/2 kinase. MEK1/2 are dual-specificity kinases, catalysing the phosphorylation of both tyrosine and threonine on ERK1 and ERK2. Phosphorylated ERK can translocate to the nucleus where it phosphorylates and activates various transcription factors (Marshall, 1996). Activated ERK1/2 catalyse the phosphorylation of numerous cytoplasmic and nuclear substrates, regulating diverse cellular responses such as mitosis, embryogenesis, cell differentiation, motility, metabolism, and programmed death, as well as angiogenesis (Shaul et al., 2007; Lewis et al., 1998; Johnson et al., 1994; D'Angelo et al., 1995; Na et al., 2010).

Factors associated with resistance to platinum include those that limit the formation of cytotoxic platinum-DNA adducts and those that prevent cell death occurring after platinum-adduct formation (Davis et al., 2014). The former may result from reduced uptake of cisplatin into cells, increased efflux via alterations to transport proteins or by inactivation of intracellular cisplatin by conversion into cisplatin-thiol conjugates. The latter form of resistance may occur by increased DNA repair after adduct formation. Alterations in various proteins associated with these repair mechanisms have been associated with platinum resistance, for example high levels of excision repair cross-complementation 1 (ERCC1) protein, mutations or down-regulation of MLH1, MSH2 and MSH1 and secondary mutations of BRCA1 or 2, which can cause reversion to the BRCA genotype and reestablishment of BRCA function, hence increasing HR (Lord and Ashworth, 2013). These various factors may either be present at diagnosis or acquired over time.

A number of studies have tried to characterise the mechanisms of acquired resistance in ovarian cancer. Analysis of 135 spatially and temporally separated samples from 14 patients with HGSOC who received platinum-based chemotherapy found that NF1 deletion showed a progressive increase in tumour allele fraction during chemotherapy (Schwarz et al., 2015). This suggested that subclonal tumour populations are present in pre-treatment biopsies in HGSOC and can undergo expansion during chemotherapy, causing clinical relapse (Schwarz et al., 2015). Additionally alteration of the NF1 gene has been associated with innate cisplatin resistance in HGSOC, whereby 20% of primary tumours showed inactivation of the NF1 gene by mutation or gene breakage (Patch et al., 2015). Furthermore mutation of the RAS-MAPK has been associated with chemotherapy resistance in relapsed neuroblastomas (Eleveld et al., 2015). Additionally, in cell line models, the MAPK pathway has been implicated in cisplatin resistance in ovarian cancer (Benedetti et al., 2008) and in squamous cell carcinoma (Kong et al., 2015).

### DESCRIPTION OF THE INVENTION

A cancer with a given histopathological diagnosis may represent multiple diseases at a molecular level. The present inventors have defined a molecular subgroup of cancer characterised by misregulation of the MAPK signalling pathway and the epithelial-mesenchymal transition (EMT) pathway. Biomarker signatures devised by the present inventors can be used to identify cancers within the molecular subgroup. The signatures are also useful for identifying the treatment that is best suited for a given patient.

Thus, in a first aspect the invention provides a method for selecting a treatment for a subject having a cancer, comprising:
(i) measuring the expression level(s) of at least 1 biomarker selected from Table A or Table B in a sample from the subject;
(ii) assessing from the expression level(s) of the at least 1 biomarker whether the sample from the subject is positive or negative for a biomarker signature comprising the at least 1 biomarker, wherein:
(a) if the sample is positive for the biomarker signature a MAPK pathway inhibitor is indicated and/or if the sample is negative for the biomarker signature a MAPK pathway inhibitor is not indicated; and/or
(b) if the sample is positive for the biomarker signature an EMT pathway inhibitor is indicated and/or if the sample is negative for the biomarker signature an EMT pathway inhibitor is not indicated; and/or
(c) if the sample is positive for the biomarker signature an SRC pathway inhibitor is not indicated and/or if the sample is negative for the biomarker signature an SRC pathway inhibitor is indicated; and/or
(d) if the sample is positive for the biomarker signature a taxane is indicated and/or if the sample is negative for the biomarker signature a taxane is not indicated.

According to a related aspect of the invention there is provided a method for selecting a treatment for a subject having a cancer, comprising:
(i) measuring the expression level(s) of at least COL5A1 and/or THBS1 in a sample from the subject;
(ii) assessing from the expression level(s) of at least COL5A1 and/or THBS1 whether the sample from the subject is positive or negative for a biomarker signature comprising COL5A1 and/or THBS1, wherein:
   if the sample is positive for the biomarker signature an anti-angiogenic therapeutic agent is indicated and/or if the sample is negative for the biomarker signature an anti-angiogenic therapeutic agent is not indicated.

According to all aspects of the invention, in specific embodiments the methods of the invention comprise measuring the expression level of THBS1. In further specific embodiments the methods of the invention comprise measuring the expression levels of COL5A1 and THBS1. In yet further specific embodiments the methods of the invention comprise measuring the expression level of COL5A1. COL5A1 and THBS1 are found in Table B herein. Thus, in addition to measuring the expression levels of at least COL5A1 and/or THBS1 the methods of the invention may include measuring one or more additional, up to all, of the biomarkers listed in Table B (optionally together with one or more biomarkers from Table A and/or one or more additional biomarkers).

The present inventors have identified that treatment of tumour cells resistant to a platinum-based chemotherapeutic agent (and positive for the biomarker signature) with a MAPK pathway inhibitor can re-sensitise the tumour cells to the platinum-based chemotherapeutic agent. Thus, in the methods described herein the MAPK pathway inhibitor may be combined with a platinum based chemotherapeutic agent. The platinum based chemotherapeutic agent may be administered before, together with, or after the MAPK pathway inhibitor. Preferably, the platinum based chemotherapeutic agent is administered together with, or after, the MAPK pathway inhibitor.

Furthermore, the present inventors have identified that the MAPK and SRC pathways act in parallel such that the inhibition of one signaling cascade leads to the activation of the other. Thus, in the methods described herein the MAPK pathway inhibitor may be combined with a SRC pathway inhibitor. The SRC pathway inhibitor may be administered before, together with, or after the MAPK pathway inhibitor. Preferably, the SRC pathway inhibitor is administered together with, or after, the MAPK pathway inhibitor.

By “indicated” is meant “indicated for treatment”, i.e. that the therapeutic agent is predicted to positively treat the cancer. A therapeutic agent is thus “indicated” if the cancer's rate of growth is expected to, or will, decelerate as a result of contact with the therapeutic agent, compared to its growth in the absence of contact with the therapeutic agent. A therapeutic agent can also be considered “indicated” if the subject's overall prognosis (progression free survival and/or overall survival) is expected to, or will, improve by administration of the therapeutic agent.

By “not indicated” is meant “not indicated for treatment”, i.e. that the therapeutic agent is predicted not to positively treat the cancer. A therapeutic agent is thus “not indicated” if the cancer's rate of growth is expected to, or will, not decelerate as a result of contact with the therapeutic agent, compared to its growth in the absence of contact with the therapeutic agent. A therapeutic agent can also be considered “not indicated” if the subject's overall prognosis (progression free survival and/or overall survival) is expected to, or will, not improve by administration of the therapeutic agent. A therapeutic agent can also be considered “not indicated” if it is “contraindicated”. By “contraindicated” is meant that a worse outcome is expected for the subject than if the subject was treated with the therapeutic agent.

According to a related aspect of the invention there is provided a method for predicting the responsiveness of a subject with cancer to a therapeutic agent comprising:
(i) measuring the expression level(s) of at least 1 biomarker(s) selected from Table A or Table B in a sample from the subject;
(ii) assessing from the expression level(s) of the at least 1 biomarker(s) whether the sample from the subject is positive or negative for a biomarker signature comprising the at least 1 biomarker;
(iii) classifying the subject as:
(a) predicted to be responsive to a MAPK pathway inhibitor if the sample is positive for the biomarker signature and/or predicted to be non-responsive to the MAPK pathway inhibitor if the sample is negative for the biomarker signature; and/or
(b) predicted to be responsive to an EMT pathway inhibitor if the sample is positive for the biomarker signature and/or predicted to be non-responsive to the EMT pathway inhibitor if the sample is negative for the biomarker signature; and/or
(c) predicted to be non-responsive to a SRC pathway inhibitor if the sample is positive for the biomarker signature and/or predicted to be responsive to the SRC inhibitor if the sample is negative for the biomarker signature; and/or
(d) predicted to be non-responsive to a platinum-based chemotherapeutic agent if the sample is positive for the biomarker signature and/or predicted to be responsive to a platinum-based chemotherapeutic agent if the sample is negative for the biomarker signature; and/or
(e) predicted to be responsive to a taxane if the sample is positive for the biomarker signature and/or predicted to be non-responsive to the taxane if the sample is negative for the biomarker signature.

There is also provided a method for predicting the responsiveness of a subject with cancer to a therapeutic agent comprising:
(i) measuring the expression level(s) of at least COL5A1 and/or THBS1 in a sample from the subject;
(ii) assessing from the expression level(s) of at least COL5A1 and/or THBS1 whether the sample from the subject is positive or negative for a biomarker signature comprising COL5A1 and/or THBS1;
(iii) classifying the subject as predicted to be responsive to an anti-angiogenic therapeutic agent if the sample is positive for the biomarker signature and/or predicted to be non-responsive to an anti-angiogenic therapeutic agent if the sample is negative for the biomarker signature.

A cancer is “responsive” to a therapeutic agent if its rate of growth is inhibited as a result of contact with the therapeutic agent, compared to its growth in the absence of contact with the therapeutic agent. Growth of a cancer can be measured in a variety of ways. For instance, the size of a tumor or measuring the expression of tumour markers appropriate for that tumour type. A cancer can also be considered responsive to a therapeutic agent if the subject's overall prognosis (progression free survival and/or overall survival) is improved by the administration of the therapeutic agent.

A cancer is “non-responsive” to a therapeutic agent if its rate of growth is not inhibited, or inhibited to a very low degree or to a non-statistically significant degree, as a result of contact with the therapeutic agent when compared to its growth in the absence of contact with the therapeutic agent. As stated above, growth of a cancer can be measured in a variety of ways, for instance, the size of a tumour or measuring the expression of tumour markers appropriate for that tumour type. A cancer can also be considered non-responsive to a therapeutic agent if the subject's overall prognosis (progression free survival and/or overall survival) is not improved by the administration of the therapeutic agent. Still further, measures of non-responsiveness can be assessed using additional criteria beyond growth size of a tumor such as, but not limited to, patient quality of life, and degree of metastases.

In yet a further aspect, the present invention relates to a method of determining clinical prognosis of a subject with cancer comprising:
(i) measuring the expression level(s) of at least 1 biomarker(s) selected from Table A or Table B in a sample from the subject;
(ii) assessing from the expression level(s) of the at least 1 biomarker(s) whether the sample from the subject is positive or negative for a biomarker signature comprising the at least 1 biomarker;
(iii) classifying the subject as having a poor prognosis if the sample is positive for the biomarker signature and/or having a good prognosis if the sample is negative for the biomarker signature.

In some embodiments, the at least 1 biomarker comprises or is COL5A1 and/or THBS1.

“Poor prognosis” may indicate decreased progression free survival and/or overall survival rates compared to samples that are negative for the biomarker signature and/or good prognosis may indicate increased progression free survival or overall survival rates compared to samples that are positive for the biomarker signature. Poor prognosis may indicate increased likelihood of recurrence or metastasis compared to samples that are negative for the biomarker signature and/or good prognosis may indicate decreased likelihood of recurrence or metastasis compared to samples that are positive for the biomarker signature. Metastasis, or metastatic disease, is the spread of a cancer from one organ or part to another non-adjacent organ or part. The new occurrences of disease thus generated are referred to as metastases.

In certain embodiments the cancer of the subject whose prognosis is determined is not a glioblastoma. In specific embodiments the cancer of the subject whose prognosis is determined is colon, lung (optionally lung adenocarcinoma) or prostate cancer (and the subject is not receiving a taxane), optionally wherein the subject with prostate cancer has been treated with radical prostatectomy and/or radical radiotherapy. The cancer may also be bladder cancer, cervical cancer, colorectal cancer, glioblastoma, head and neck cancer, renal cancer (optionally renal clear cell or renal papillary cancer), glioma (optionally lower grade glioma), pancreatic cancer, melanoma, ovarian cancer and/or stomach cancer. In certain embodiments the subject is receiving, has received and/or will receive treatment with the standard of care treatment. A skilled practitioner is aware of the standard of care treatment for the particular cancer. In some embodiments, the standard of care treatment incorporates a platinum-based chemotherapeutic agent (as defined herein).

The signatures disclosed herein provide a prognostic indication. This may apply to untreated patients. It may also apply to patients treated with standard of care treatment.

However, the signatures disclosed herein also predict responsiveness to particular targeted, or indicated, therapeutic agents. Accordingly, in specific embodiments a subject with cancer whose sample is positive for the biomarker signature may have a better prognosis when treated with an indicated therapeutic agent to which they are predicted to be responsive than a subject with a cancer whose sample is negative for the biomarker signature and who is treated with the same therapeutic agent. For example, it is shown herein that subjects with (de novo) metastatic prostate cancer who are signature positive have a good prognosis (increased overall survival) relative to signature negative subjects when treated with a taxane. Thus, according to all aspects of the invention, a subject that is signature positive may be selected for therapy and this improves their prognosis. Alternatively, a subject that is signature negative has an improved prognosis and thus is not selected for therapy.

According to a further aspect the invention provides a method for selecting a treatment for a subject having a cancer, comprising:
(i) measuring the expression level(s) of at least 1 biomarker selected from Table A or Table B in a sample from the subject;
(ii) assessing from the expression level(s) of the at least 1 biomarker whether the sample from the subject is positive or negative for a biomarker signature comprising the at least 1 biomarker, wherein an increased expression level and/or a decreased expression level of the at least 1 biomarker as shown in Tables C and D indicates that the sample is positive or negative for the biomarker signature, wherein:
(a) if the sample is positive for the biomarker signature a MAPK pathway inhibitor is indicated and/or if the sample is negative for the biomarker signature a MAPK pathway inhibitor is not indicated; and/or
(b) if the sample is positive for the biomarker signature an EMT pathway inhibitor is indicated and/or if the sample is negative for the biomarker signature an EMT pathway inhibitor is not indicated; and/or
(c) if the sample is positive for the biomarker signature an SRC pathway inhibitor is not indicated and/or if the sample is negative for the biomarker signature an SRC pathway inhibitor is indicated.

In some embodiments, the at least 1 biomarker comprises or is COL5A1 and/or THBS1. By “treatment” is meant any therapy or surgery that may be provided to a subject in order to improve, stabilise, or minimise deterioration of a medical condition. The condition relevant to the present invention is cancer (in particular a cancer of the type indicated herein). Means of administration of therapeutic agents include oral, rectal, sublingual, sublabial, intravenous, intraatricular, intracardiac, intracavernous, intramuscular, epidural, intracerebral, intracerebroventricular, epicutaneous, nasal, intrathecal, or via a gastral or duodenal feeding tube and are known in the art. Similarly dosage forms and dosage regimes are known for therapeutic agents and can be determined by a practising physician. Therapeutic agents are approved and marketed for administration in a given dosage form, together with detailed prescribing instructions. Thus, the invention is not limited in relation to how, or in what form, the therapeutic agent is administered since the skilled person would be in a position to determine this based on the therapeutic agent of interest.

TABLE A
Weighting and bias for the 45 gene signature
45 Gene Signature

| Rank | Gene Name | Weight | Bias |
|---|---|---|---|
| 1 | TMEM200A | 0.059481295 | 3.681329367 |
| 2 | GJB2 | 0.055985433 | 4.479833955 |
| 3 | MMP13 | 0.038284076 | 3.724107067 |
| 4 | GFPT2 | 0.037990641 | 4.860237265 |
| 5 | POSTN | −0.035480409 | 4.359882019 |
| 6 | BICC1 | 0.030426737 | 3.698203663 |
| 7 | CDH11 | 0.028340142 | 4.996780524 |
| 8 | MRVI1 | 0.025598535 | 5.076083782 |
| 9 | PMP22 | 0.024034610 | 5.564463361 |
| 10 | COL11A1 | −0.023672753 | 3.500170591 |
| 11 | IGFL2 | 0.022225316 | 3.310383445 |
| 12 | LUM | −0.022014619 | 8.336273473 |
| 13 | NTM | −0.021750365 | 4.230245127 |
| 14 | BGN | 0.021089508 | 10.15236225 |
| 15 | COL3A1 | −0.021023256 | 8.323635399 |
| 16 | COL10A1 | 0.019650845 | 6.353832828 |
| 17 | RAB31 | 0.018014921 | 5.317119481 |
| 18 | ANGPTL2 | 0.016630934 | 5.639562288 |
| 19 | PLAU | 0.016596202 | 5.848820224 |
| 20 | COL8A1 | 0.016373799 | 6.419330171 |
| 21 | MIR1245 | 0.015290888 | 5.455187262 |
| 22 | POLD2 | 0.014555548 | 9.38782491 |
| 23 | NKD2 | 0.014468847 | 7.371707677 |
| 24 | FZD1 | 0.014334768 | 4.151874676 |
| 25 | COPZ2 | 0.013866713 | 5.103944696 |
| 26 | ITGA5 | 0.013561913 | 8.36627973 |
| 27 | VGLL3 | 0.012488674 | 4.501866677 |
| 28 | INHBA | −0.011763261 | 4.684272993 |
| 29 | MMP14 | 0.011010832 | 10.08406264 |
| 30 | VCAN | 0.009977966 | 5.551759846 |
| 31 | THBS2 | −0.008700202 | 8.130920944 |
| 32 | RUNX2 | 0.008333275 | 4.73450528 |
| 33 | TIMP3 | 0.008141253 | 6.498316457 |
| 34 | SFRP2 | −0.007890741 | 5.601725816 |
| 35 | COL1A2 | 0.007788938 | 6.01000198 |
| 36 | COL5A2 | −0.007217722 | 3.567060064 |
| 37 | SERPINF1 | 0.006801251 | 10.8333948 |
| 38 | KIF26B | −0.005249312 | 4.97815094 |
| 39 | TNFAIP6 | 0.004963450 | 5.361760185 |
| 40 | MMP2 | 0.003988003 | 5.362247865 |
| 41 | FN1 | 0.003130435 | 4.984016427 |
| 42 | ALPK2 | 0.002394440 | 3.513604572 |
| 43 | CTSK | 0.001542586 | 5.732155915 |
| 44 | LOXL1 | −0.001415170 | 9.593869933 |
| 45 | FAP | −0.000007237 | 5.23E+00 |

TABLE B
Weighting and bias for the 15 gene signature
15 Gene Signature

| Rank | Gene Name | Entrez Gene ID | Weight | Bias |
|---|---|---|---|---|
| 1 | GJB2 | 2706 | 0.089719778 | 4.478098614 |
| 2 | CDH11 | 1009 | 0.066544238 | 4.990055702 |
| 3 | GFPT2 | 9945 | 0.058421032 | 4.885349473 |
| 4 | COL10A1 | 1300 | 0.040148445 | 6.357258041 |
| 5 | ANGPTL2 | 23452 | 0.038272311 | 5.631697532 |
| 6 | THBS1 | 7057 | 0.036613387 | 6.428114883 |
| 7 | RAB31 | 11031 | 0.033158407 | 5.300536304 |
| 8 | THBS2 | 7058 | −0.030849235 | 8.135441538 |
| 9 | INHBA | 3624 | −0.028500708 | 4.68290899 |
| 10 | MMP14 | 4323 | 0.020727894 | 10.07844987 |
| 11 | VCAN | 1462 | 0.020706504 | 5.529961284 |
| 12 | PLAU | 5328 | 0.019342831 | 5.850016491 |
| 13 | COL5A1 | 1289 | 0.010674165 | 5.569094517 |
| 14 | FAP | 2191 | −0.006101691 | 5.226391586 |
| 15 | FN1 | 2335 | −0.005998124 | 4.982941989 |

According to all aspects of the invention an increased expression level and/or a decreased expression level of the biomarker(s) may contribute to the determination that the sample is positive or negative for the biomarker signature. As shown in Tables C and D, a threshold level of gene expression can be set and a value above or below that threshold may then indicate increased or decreased expression levels. Of course, the invention is not limited to the specific values; the skilled person would appreciate that the suitable values may be determined depending upon the data set in question.

TABLE C
Up/Down Regulation in signature positive and negative groups for the 45 gene signature

| 45 Gene | | R0171 | | | ICON7 | | | Tothill | | |
|---|---|---|---|---|---|---|---|---|---|---|
| Signature | | Up/Down Regulation | | | Up/Down Regulation | | | Up/Down Regulation | | |
| | Gene | Median | Sig | Sig | Median | Sig | Sig | Median | Sig | Sig |
| Rank | Name | Threshold | Pos | Neg | Threshold | Pos | Neg | Threshold | Pos | Neg |
| 1 | TMEM200A | 3.6466 | 5.0657 | 2.968975 | 3.431955 | 5.07791 | 2.68994 | 4.75751 | 6.21209 | 4.579655 |
| 2 | GJB2 | 4.2951 | 6.2298 | 3.22823 | 4.8581 | 6.37259 | 3.81911 | 5.42481 | 8.50465 | 5.246225 |
| 3 | MMP13 | 2.5777 | 5.3747 | 2.147585 | 2.100465 | 5.42182 | 1.88248 | 2.72939 | 5.18685 | 2.66861 |
| 4 | GFPT2 | 5.0409 | 5.8341 | 4.445485 | 4.98664 | 5.52659 | 4.3683 | 4.47669 | 5.48663 | 4.34832 |
| 5 | POSTN | 3.9196 | 6.0564 | 3.111495 | 3.89304 | 5.37474 | 3.44404 | 6.0011 | 8.89395 | 5.52013 |
| 6 | BICC1 | 3.6377 | 4.3439 | 3.25068 | 3.86463 | 4.46272 | 3.52687 | 3.52687 | 4.60078 | 3.41863 |
| 7 | CDH11 | 4.959 | 5.9437 | 4.50373 | 4.97344 | 5.70289 | 4.47351 | 4.7575 | 5.75619 | 4.573065 |
| 8 | MRVI1 | 5.0188 | 5.896 | 4.67305 | 5.83413 | 6.37259 | 5.66073 | 4.9964 | 6.07559 | 4.85401 |
| 9 | PMP22 | 5.4884 | 6.0903 | 5.227205 | 5.321515 | 5.95533 | 4.9538 | 4.5429 | 5.08954 | 4.462555 |
| 10 | COL11A1 | 3.1807 | 5.2886 | 2.57622 | 2.55951 | 3.73033 | 2.26097 | 5.7018 | 9.63595 | 5.3606 |
| 11 | IGFL2 | 3.1305 | 3.9419 | 2.7999 | 3.11509 | 3.60992 | 2.93275 | 4.47336 | 6.6255 | 4.37059 |
| 12 | LUM | 8.3049 | 9.8639 | 7.72196 | 7.754525 | 9.50299 | 7.22957 | 8.44188 | 10.9589 | 8.20698 |
| 13 | NTM | 4.1848 | 5.5422 | 3.40771 | 4.744045 | 5.94384 | 4.18479 | 4.47668 | 6.64725 | 4.124785 |
| 14 | BGN | 10.123 | 11.055 | 9.72598 | 9.636855 | 10.6681 | 9.26884 | 7.03593 | 9.26754 | 6.88147 |
| 15 | COL3A1 | 8.4315 | 9.6558 | 7.62774 | 9.130145 | 10.5306 | 8.63779 | 11.3205 | 12.3325 | 11.1339 |
| 16 | COL10A1 | 6.1925 | 7.639 | 5.687805 | 6.18854 | 7.62534 | 5.67381 | 5.67381 | 8.43772 | 5.350655 |
| 17 | RAB31 | 5.2259 | 6.0563 | 4.85749 | 4.87908 | 5.26397 | 4.75062 | 7.84569 | 9.69752 | 7.72347 |
| 18 | ANGPTL2 | 5.5728 | 6.1959 | 5.35038 | 6.13942 | 6.79376 | 5.80861 | 4.50872 | 5.3606 | 4.473445 |
| 19 | PLAU | 5.7826 | 7.0685 | 5.39088 | 6.085085 | 7.02735 | 5.75619 | 6.37259 | 8.86471 | 6.14652 |
| 20 | COL8A1 | 6.2628 | 7.3746 | 5.92596 | 6.146445 | 7.3527 | 5.83413 | 2.18156 | 4.50373 | 2.04513 |
| 21 | MIR1245 | 5.4218 | 6.9783 | 4.613905 | 4.42027 | 5.74407 | 3.6255 | 6.45712 | 9.67813 | 6.20229 |
| 22 | POLD2 | 9.4897 | 10.075 | 8.97053 | 9.91729 | 10.2823 | 9.71438 | 6.58238 | 6.0885 | 6.64004 |
| 23 | NKD2 | 7.3833 | 8.3311 | 6.710345 | 6.20246 | 7.38327 | 5.74407 | 5.72925 | 6.37317 | 5.59965 |
| 24 | FZD1 | 4.1376 | 4.7122 | 3.835685 | 4.18478 | 4.67879 | 3.87365 | 5.74279 | 6.48761 | 5.64527 |
| 25 | COPZ2 | 5.022 | 5.7167 | 4.793585 | 4.4387 | 4.84834 | 4.28628 | 5.14939 | 6.10955 | 5.06428 |
| 26 | ITGA5 | 8.3489 | 9.247 | 7.84255 | 7.075035 | 7.70612 | 6.74078 | 6.02345 | 7.05465 | 5.93189 |
| 27 | VGLL3 | 4.7684 | 5.8592 | 3.929875 | 4.283195 | 5.6291 | 3.0979 | 3.50074 | 5.65796 | 3.238385 |
| 28 | INHBA | 4.799 | 5.8341 | 3.95036 | 4.15386 | 5.55123 | 3.6377 | 5.75619 | 8.93272 | 5.35302 |
| 29 | MMP14 | 10.15 | 10.959 | 9.74279 | 10.1416 | 10.9561 | 9.75752 | 5.5194 | 6.22976 | 5.463645 |
| 30 | VCAN | 5.6749 | 6.9728 | 5.02986 | 6.0345 | 7.40045 | 5.30103 | 7.48408 | 10.3997 | 6.896115 |
| 31 | THBS2 | 8.2106 | 9.91 | 7.244625 | 8.12437 | 9.68603 | 7.14035 | 7.98033 | 11.2575 | 7.53488 |
| 32 | RUNX2 | 4.7684 | 5.5194 | 4.45994 | 4.9964 | 5.52013 | 4.78477 | 3.71929 | 4.71445 | 3.616915 |
| 33 | TIMP3 | 6.6328 | 7.618 | 6.148145 | 6.65443 | 7.52754 | 6.07098 | 6.33734 | 8.3709 | 6.20779 |
| 34 | SFRP2 | 5.1867 | 6.4853 | 4.87875 | 4.96453 | 6.77789 | 4.57074 | 6.1626 | 10.7345 | 5.687825 |
| 35 | COL1A2 | 6.0235 | 6.8854 | 5.5466 | 5.85296 | 6.92015 | 5.6464 | 11.7545 | 12.4163 | 11.5546 |
| 36 | COL5A2 | 3.3203 | 4.7575 | 2.7999 | 2.109115 | 3.45142 | 1.93264 | 7.58833 | 10.9619 | 7.378935 |
| 37 | SERPINF1 | 10.852 | 11.276 | 10.542 | 10.5045 | 10.85 | 10.3568 | 8.23385 | 10.7163 | 7.971665 |
| 38 | KIF26B | 5.0465 | 5.8838 | 4.646265 | 4.487515 | 5.6464 | 4.18041 | 4.02287 | 5.18685 | 3.94881 |
| 39 | TNFAIP6 | 5.6232 | 7.0236 | 4.696 | 5.39467 | 6.28051 | 4.50373 | 5.02198 | 6.66868 | 4.73379 |
| 40 | MMP2 | 5.0657 | 6.1321 | 4.7595 | 5.56188 | 6.41938 | 5.08709 | 6.89652 | 9.34124 | 6.692765 |
| 41 | FN1 | 5.0465 | 6.2104 | 4.43652 | 4.83753 | 6.29804 | 4.58857 | 10.6461 | 11.8498 | 10.36865 |
| 42 | ALPK2 | 3.1273 | 4.4405 | 2.636575 | 3.005285 | 3.87365 | 2.84256 | 3.50074 | 5.45585 | 3.41863 |
| 43 | CTSK | 5.6453 | 7.2439 | 5.14669 | 6.0665 | 7.53176 | 5.54197 | 7.46379 | 10.6461 | 7.21515 |
| 44 | LOXL1 | 9.6781 | 10.522 | 9.18543 | 9.40259 | 10.269 | 9.07973 | 6.83225 | 8.48727 | 6.658005 |
| 45 | FAP | 5.4623 | 7.0793 | 4.05782 | 5.05518 | 6.94192 | 3.94881 | 6.06714 | 8.72705 | 5.65024 |

TABLE D
Up/Down Regulation in signature positive and negative groups for the 15 gene signature

| 15 Gene | | R0171 | | | ICON7 | | | Tothill | | |
|---|---|---|---|---|---|---|---|---|---|---|
| Signature | | Up/Down Regulation | | | Up/Down Regulation | | | Up/Down Regulation | | |
| | Gene | Median | Sig | Sig | Median | Sig | Sig | Median | Sig | Sig |
| Rank | Name | Threshold | Pos | Neg | Threshold | Pos | Neg | Threshold | Pos | Neg |
| 1 | GJB2 | 4.33026 | 6.48402 | 3.25515 | 4.85745 | 6.19138 | 3.21176 | 5.35967 | 7.58391 | 4.50547 |
| 2 | CDH11 | 4.95514 | 5.957695 | 4.57074 | 4.97344 | 5.44587 | 4.45419 | 5.86851 | 6.98271 | 5.38762 |
| 3 | GFPT2 | 5.0409 | 5.908965 | 4.51315 | 5.009845 | 5.4989 | 4.02287 | 4.90305 | 5.82236 | 4.46339 |
| 4 | COL10A1 | 6.17841 | 7.691215 | 5.73055 | 6.179605 | 7.29219 | 5.54159 | 5.59965 | 7.79347 | 4.70377 |
| 5 | ANGPTL2 | 5.57261 | 6.272925 | 5.35491 | 6.152555 | 6.79748 | 5.75619 | 4.58191 | 5.32149 | 4.27131 |
| 6 | THBS1 | 6.42853 | 7.53905 | 6.05232 | 7.087685 | 7.96919 | 6.64004 | 3.88865 | 4.79113 | 3.47753 |
| 7 | RAB31 | 5.20634 | 6.124385 | 4.86097 | 4.854735 | 5.24978 | 4.64093 | 7.89046 | 9.1462 | 7.38142 |
| 8 | THBS2 | 8.20969 | 9.916045 | 7.25212 | 8.197455 | 9.54184 | 6.99702 | 7.85242 | 10.0919 | 6.65443 |
| 9 | INHBA | 4.7808 | 5.934 | 3.92521 | 4.1656 | 5.44265 | 3.39663 | 5.64527 | 7.70293 | 4.75757 |
| 10 | MMP14 | 10.1356 | 11.0802 | 9.73076 | 10.12305 | 10.687 | 9.58809 | 5.462 | 5.7299 | 5.32419 |
| 11 | VCAN | 5.68904 | 7.053055 | 5.0409 | 6.0165 | 7.09525 | 5.04218 | 7.37274 | 9.14764 | 6.30121 |
| 12 | PLAU | 5.78374 | 7.113495 | 5.36554 | 6.085085 | 6.90371 | 5.54197 | 6.27759 | 7.92671 | 5.71559 |
| 13 | COL5A1 | 5.34396 | 6.458105 | 5.06873 | 5.4289 | 6.1982 | 5.14939 | 6.9052 | 8.47557 | 6.24942 |
| 14 | FAP | 5.462 | 7.256835 | 4.06058 | 5.0628 | 6.82319 | 3.69681 | 5.98698 | 8.11692 | 4.83618 |
| 15 | FN1 | 5.04218 | 6.220655 | 4.43401 | 4.83223 | 6.02411 | 4.4343 | 10.9984 | 12.3804 | 10.276 |

The biomarker signature may be defined by the probesets listed in Tables E and F and by the expression levels of the corresponding genes, which may be measured using the probesets. The biomarker signature may include the expression levels of one or more additional biomarkers, which may be measured in any suitable way, for example using one or more additional probesets.

TABLE E
45 gene signature probeset information

| | | | No. | | | Entrez | | | | SEQ |
|---|---|---|---|---|---|---|---|---|---|---|
| Probe | | | probes | Ensemble | Gene | Gene | | Chromo- | | ID |
| Set ID | Type | Orientation | aligned | Gene | Symbol | ID | Description | some | Strand | NO. |
| OCAD | Expres- | Sense | 11 | ENS | AL | 115701 | alpha-kinase 2 [Source: HGNC | Chr 18 | Reverse | 1 |
| NP.123 | sion | (includes | | G000 | PK | | Symbol; Acc: 20565] | | Strand | |
| 61_s_at | probe | Intronic) | | 0019 | 2 | | | | | |
| | set | | | 8796 | | | | | | |
| OC3SN | Expres- | Sense | 11 | ENS | AL | 115701 | alpha-kinase 2 [Source: HGNC | Chr 18 | Reverse | 2 |
| 0.596- | sion | (Fully | | G000 | PK | | Symbol; Acc: 20565] | | Strand | |
| 9a_s_at | probe | Exonic) | | 0019 | 2 | | | | | |
| | set | | | 8796 | | | | | | |
| OC3P. | Expres- | Sense | 11 | ENS | AN | 23452 | angiopoietin-like 2 | Chr 9 | Reverse | 3 |
| 2679.0 | sion | (Fully | | G000 | GP | | [Source: HGNC | | Strand | |
| 1_s_at | probe | Exonic) | | 0013 | TL2 | | Symbol; Acc: 490] | | | |
| | set | | | 6859 | | | | | | |
| ADXStr | Almac | Sense | 1 | ENS | CY | 1545 | cytochrome P450, family 1, | Chr 2 | Reverse | N/A |
| ongB | pixe- | (Fully | | G000 | P1 | | subfamily B, polypeptide 1 | | Strand | |
| 12_at | llation | Exonic) | | 0013 | B1 | | [Source: HGNC | | | |
| | control | | | 8061 | | | Symbol; Acc: 2597] | | | |
| ADXStr | Almac | Sense | 1 | ENS | KA | 7994 | K(lysine) acetyltransferase 6A | Chr 8 | Reverse | N/A |
| ongB | pixe- | (Fully | | G000 | T6 | | [Source: HGNC | | Strand | |
| 12_at | llation | Exonic) | | 0008 | A | | Symbol; Acc: 13013] | | | |
| | control | | | 3168 | | | | | | |
| ADXStr | Almac | Sense | 1 | ENS | NC | 8440 | NCK adaptor protein 2 | Chr 2 | Forward | N/A |
| ongB | pixe- | (Fully | | G000 | K2 | | [Source: HGNC | | Strand | |
| 12_at | llation | Exonic) | | 0007 | | | Symbol; Acc: 7665] | | | |
| | control | | | 1051 | | | | | | |
| ADXStr | Almac | Sense | 1 | ENS | AN | 23452 | angiopoietin-like 2 | Chr 9 | Reverse | N/A |
| ongB | pixe- | (Fully | | G000 | GP | | [Source: HGNC | | Strand | |
| 12_at | llation | Exonic) | | 0013 | TL2 | | Symbol; Acc: 490] | | | |
| | control | | | 6859 | | | | | | |
| ADXStr | Almac | Sense | 1 | ENS | NR | 4900 | neurogranin (protein kinase C | Chr 11 | Forward | N/A |
| ongB | pixe- | (Fully | | G000 | GN | | substrate, RC3) [Source: HGNC | | Strand | |
| 12_at | llation | Exonic) | | 0015 | | | Symbol; Acc: 8000] | | | |
| | control | | | 4146 | | | | | | |
| ADXStr | Almac | Sense | 1 | ENS | AT | 489 | ATPase, Ca++ transporting, | Chr 17 | Reverse | N/A |
| ongB | pixe- | (Fully | | G000 | P2 | | ubiquitous [Source: HGNC | | Strand | |
| 12_at | llation | Exonic) | | 0007 | A3 | | Symbol; Acc: 813] | | | |
| | control | | | 4370 | | | | | | |
| ADXStr | Almac | Sense | 1 | ENS | AN | 88455 | ankyrin repeat domain 13A | Chr 12 | Forward | N/A |
| ongB | pixe- | (Fully | | G000 | KR | | [Source: HGNC | | Strand | |
| 12_at | llation | Exonic) | | 0007 | D1 | | Symbol; Acc: 21268] | | | |
| | control | | | 6513 | 3A | | | | | |
| ADXStr | Almac | Sense | 1 | ENS | MO | 81532 | MOB kinase activator 2 | Chr 11 | Reverse | N/A |
| ongB | pixe- | (Fully | | G000 | B2 | | [Source: HGNC | | Strand | |
| 12_at | llation | Exonic) | | 0018 | | | Symbol; Acc: 24904] | | | |
| | control | | | 2208 | | | | | | |
| ADXStr | Almac | Sense | 1 | ENS | PT | 80142 | prostaglandin E synthase 2 | Chr 9 | Reverse | N/A |
| ongB | pixe- | (Fully | | G000 | GE | | [Source: HGNC | | Strand | |
| 12_at | llation | Exonic) | | 0014 | S2 | | Symbol; Acc: 17822] | | | |
| | control | | | 8334 | | | | | | |
| ADXStr | Almac | Sense | 1 | ENS | ZF | 677 | ZFP36 ring finger protein-like 1 | Chr 14 | Reverse | N/A |
| ongB | pixe- | (Fully | | G000 | P36 | | [Source: HGNC | | Strand | |
| 12_at | llation | Exonic) | | 0018 | L1 | | Symbol; Acc: 1107] | | | |
| | control | | | 5650 | | | | | | |
| OC3P. | Expres- | Sense | 11 | ENS | AN | 23452 | angiopoietin-like 2 | Chr 9 | Reverse | 4 |
| 9834. | sion | (Fully | | G000 | GP | | [Source: HGNC | | Strand | |
| C1_s_at | probe | Exonic) | | 0013 | TL2 | | Symbol; Acc: 490] | | | |
| | set | | | 6859 | | | | | | |
| OCMX. | Expres- | Sense | 1 | ENS | AN | 23452 | angiopoietin-like 2 | Chr 9 | Reverse | 5 |
| 9546. | sion | (Fully | | G000 | GP | | [Source: HGNC | | Strand | |
| C1_x_at | probe | Exonic) | | 0013 | TL2 | | Symbol; Acc: 490] | | | |
| | set | | | 6859 | | | | | | |
| OCAD | Expres- | Sense | 11 | ENS | AN | 23452 | angiopoietin-like 2 | Chr 9 | Reverse | 6 |
| A.82 | sion | (includes | | G000 | GP | | [Source: HGNC | | Strand | |
| 26_s_at | probe | Intronic) | | 0013 | TL2 | | Symbol; Acc: 490] | | | |
| | set | | | 6859 | | | | | | |
| OCAD | Expres- | Sense | 8 | ENS | AN | 23452 | angiopoietin-like 2 | Chr 9 | Reverse | 7 |
| NP.881 | sion | (Fully | | G000 | GP | | [Source: HGNC | | Strand | |
| 1_s_at | probe | Exonic) | | 0013 | TL2 | | Symbol; Acc: 490] | | | |
| | set | | | 6859 | | | | | | |
| OCHP. | Expres- | Sense | 11 | ENS | BG | 633 | biglycan [Source: HGNC | Chr X | Forward | 8 |
| 937_s_ | sion | (Fully | | G000 | N | | Symbol; Acc: 1044] | | Strand | |
| at | probe | Exonic) | | 0018 | | | | | | |
| | set | | | 2492 | | | | | | |
| OCAD | Expres- | Sense | 11 | ENS | BG | 633 | biglycan [Source: HGNC | Chr X | Forward | 9 |
| NP.988 | sion | (Fully | | G000 | N | | Symbol; Acc: 1044] | | Strand | |
| 3_s_at | probe | Exonic) | | 0018 | | | | | | |
| | set | | | 2492 | | | | | | |
| ADXStr | Almac | Sense | 1 | ENS | LE | 54741 | leptin receptor overlapping | Chr 1 | Forward | N/A |
| ong61_ | pixe- | (Fully | | G000 | PR | | transcript [Source: HGNC ward | | Strand | |
| at | llation | Exonic) | | 0021 | OT | | Symbol; Acc: 29477] | | | |
| | control | | | 3625 | | | | | | |
| ADXStr | Almac | Sense | 1 | ENS | PL | 55111/// | pleckstrin homology domain | Chr 19 | Reverse | N/A |
| ong61_ | pixe- | (Fully | | G000 | EK | 102466 | containing, family J member 1 | | Strand | |
| at | llation | Exonic) | | 0010 | HJ1 | 736 | [Source: HGNC | | | |
| | control | | | 4886 | | | Symbol; Acc: 18211] | | | |
| ADXStr | Almac | Sense | 1 | ENS | NM | 4830 | NME/NM23 nucleoside | Chr 17 | Forward | N/A |
| ong61_ | pixe- | (Fully | | G000 | E1 | | diphosphate kinase 1 | | Strand | |
| at | llation | Exonic) | | 0023 | | | [Source: HGNC | | | |
| | control | | | 9672 | | | Symbol; Acc: 7849] | | | |
| ADXStr | Almac | Sense | 1 | ENS | UB | 7329 | ubiquitin-conjugating enzyme | Chr 16 | Forward | N/A |
| ong61_ | pixe- | (Fully | | G000 | E2I | | E21 [Source: HGNC | | Strand | |
| at | llation | Exonic) | | 0010 | | | Symbol; Acc: 12485] | | | |
| | control | | | 3275 | | | | | | |
| ADXStr | Almac | Sense | 1 | ENS | CE | 55125 | centrosomal protein 192 kDa | Chr 18 | Forward | N/A |
| ong61_ | pixe- | (Fully | | G000 | P19 | | [Source: HGNC | | Strand | |
| at | llation | Exonic) | | 0010 | 2 | | Symbol; Acc: 25515] | | | |
| | control | | | 1639 | | | | | | |
| ADXStr | Almac | Sense | 1 | ENS | BG | 633 | biglycan [Source: HGNC | Chr X | Forward | N/A |
| ong61_ | pixe- | (Fully | | G000 | N | | Symbol; Acc: 1044] | | Strand | |
| at | llation | Exonic) | | 0018 | | | | | | |
| | control | | | 2492 | | | | | | |
| ADXStr | Almac | Sense | 1 | ENS | SC | 55681 | SCY1-like 2(S. cerevisiae) | Chr 12 | Forward | N/A |
| ong61_ | pixe- | (Fully | | G000 | YL2 | | [Source: HGNC | | Strand | |
| at | llation | Exonic) | | 0013 | | | Symbol; Acc: 19286] | | | |
| | control | | | 6021 | | | | | | |
| ADXStr | Almac | Sense | 1 | ENS | PR | 5589 | protein kinase C substrate | Chr 19 | Forward | N/A |
| ong61_ | pixe- | (Fully | | G000 | KCH | | 80K—H [Source: HGNC | | Strand | |
| at | llation | Exonic) | | 0013 | SH | | Symbol; Acc: 9411] | | | |
| | control | | | 0175 | | | | | | |
| ADXStr | Almac | Sense | 1 | ENS | MI | 100302 | microRNA 1227 [Source: H | Chr 19 | Reverse | N/A |
| ong61_ | pixe- | (Fully | | G000 | R1 | 283 | GNC Symbol; Acc: 33932] | | Strand | |
| at | llation | Exonic) | | 0022 | 227 | | | | | |
| | control | | | 1411 | | | | | | |
| ADXStr | Almac | Sense | 1 | ENS | CA | 91860 | calmodulin-like 4 | Chr 15 | Reverse | N/A |
| ong61_ | pixe- | (Fully | | G000 | LM | | [Source: HGNC | | Strand | |
| at | llation | Exonic) | | 0012 | L4 | | Symbol; Acc: 18445] | | | |
| | control | | | 9007 | | | | | | |
| ADXStr | Almac | Sense | 1 | ENS | N/A | N/A | Uncharacterized protein | Chr 15 | Reverse | N/A |
| ong61_ | pixe- | (Fully | | G000 | | | [Source: UniProtKB/TrEMBL; | | Strand | |
| at | llation | Exonic) | | 0026 | | | Acc: H3BRN7] | | | |
| | control | | | 0007 | | | | | | |
| OCAD | Expres- | Sense | 11 | ENS | BIC | 80114 | bicaudal C homolog 1 | Chr 10 | Forward | 10 |
| NP.195 | sion | (includes | | G000 | C1 | | (Drosophila) [Source: HGNC | | Strand | |
| 0_s_at | probe | Intronic) | | 0012 | | | Symbol; Acc: 19351] | | | |
| | set | | | 2870 | | | | | | |
| OCAD | Expre- | Sense | 11 | ENS | BIC | 80114 | bicaudal C homolog 1 | Chr 10 | Forward | 11 |
| A.1038 | sion | (Fully | | G000 | C1 | | (Drosophila) [Source: HGNC | | Strand | |
| 8_s_at | probe | Exonic) | | 0012 | | | Symbol; Acc: 19351] | | | |
| | set | | | 2870 | | | | | | |
| OCMX | Expre- | Sense | 6 | ENS | BIC | 80114 | bicaudal C homolog 1 | Chr 10 | Forward | 12 |
| SNG.4 | sion | (includes | | G000 | C1 | | (Drosophila) [Source: HGNC | | Strand | |
| 199_x_ | probe | Intronic) | | 0012 | | | Symbol; Acc: 19351] | | | |
| at | set | | | 2870 | | | | | | |
| OC3SN | Expre- | Sense | 11 | ENS | BIC | 80114 | bicaudal C homolog 1 | Chr 10 | Forward | 13 |
| Gnh.70 | sion | (Fully | | G000 | C1 | | (Drosophila) [Source: HGNC | | Strand | |
| 31_s_at | probe | Exonic) | | 0012 | | | Symbol; Acc: 19351] | | | |
| | set | | | 2870 | | | | | | |
| OCRS2. | Expre- | Sense | 11 | ENS | BIC | 80114 | bicaudal C homolog 1 | Chr 10 | Forward | 14 |
| 4990_ | sion | (Fully | | G000 | C1 | | (Drosophila) [Source: HGNC | | Strand | |
| s_at | probe | Exonic) | | 0012 | | | Symbol; Acc: 19351] | | | |
| | set | | | 2870 | | | | | | |
| OC3SN | Expre- | Sense | 11 | ENS | BIC | 80114 | bicaudal C homolog 1 | Chr 10 | Forward | 15 |
| Gnh.67 | sion | (includes | | G000 | C1 | | (Drosophila) [Source: HGNC | | Strand | |
| 78_s_at | probe | Intronic) | | 0012 | | | Symbol; Acc: 19351] | | | |
| | set | | | 2870 | | | | | | |
| OC3SN | Expre- | Sense | 11 | ENS | BIC | 80114 | bicaudal C homolog 1 | Chr 10 | Forward | 16 |
| Gnh.11 | sion | (includes | | G000 | C1 | | (Drosophila) [Source: HGNC | | Strand | |
| 887_x_ | probe | Intronic) | | 0012 | | | Symbol; Acc: 19351] | | | |
| at | set | | | 2870 | | | | | | |
| OC3P. | Expre- | Sense | 11 | ENS | CD | 1009 | cadherin 11, type 2, OB- | Chr 16 | Reverse | 17 |
| 14147. | sion | (Fully | | G000 | H1 | | cadherin (osteoblast) | | Strand | |
| C1_s_at | probe | Exonic) | | 0014 | 1 | | [Source: HGNC | | | |
| | set | | | 0937 | | | Symbol; Acc: 1750] | | | |
| OCAD | Expre- | Sense | 11 | ENS | CD | 1009 | cadherin 11, type 2, OB- | Chr 16 | Reverse | 18 |
| NP.100 | sion | (Fully | | G000 | H1 | | cadherin (osteoblast) | | Strand | |
| 24_s_a | probe | Exonic) | | 0014 | 1 | | [Source: HGNC | | | |
| t | set | | | 0937 | | | Symbol; Acc: 1750] | | | |
| OCHP. | Expre- | Sense | 11 | ENS | CD | 1009 | cadherin 11, type 2, OB- | Chr 16 | Reverse | 19 |
| 148_s_ | sion | (Fully | | G000 | H1 | | cadherin (osteoblast) | | Strand | |
| at | probe | Exonic) | | 0014 | 1 | | [Source: HGNC | | | |
| | set | | | 0937 | | | Symbol; Acc: 1750] | | | |
| OCAD | Expre- | Sense | 9 | ENS | CD | 1009 | cadherin 11, type 2, OB- | Chr 16 | Reverse | 20 |
| A.62 | sion | (includes | | G000 | H1 | | cadherin (osteoblast) | | Strand | |
| 10_s_at | probe | Intronic) | | 0014 | 1 | | [Source: HGNC | | | |
| | set | | | 0937 | | | Symbol; Acc: 1750] | | | |
| OC3SN | Expre- | Sense | 6 | ENS | CD | 1009 | cadherin 11, type 2, OB- | Chr 16 | Reverse | 21 |
| Gnh.50 | sion | (includes | | G000 | H1 | | cadherin (osteoblast) | | Strand | |
| 56_x_at | probe | Intronic) | | 0014 | 1 | | [Source: HGNC | | | |
| | set | | | 0937 | | | Symbol; Acc: 1750] | | | |
| OC3SN | Expre- | Sense | 11 | ENS | CD | 1009 | cadherin 11, type 2, OB- | Chr 16 | Reverse | 22 |
| Gnh.40 | sion | (includes | | G000 | H1 | | cadherin (osteoblast) | | Strand | |
| 32_s_at | probe | Intronic) | | 0014 | 1 | | [Source: HGNC | | | |
| | set | | | 0937 | | | Symbol; Acc: 1750] | | | |
| OCHP | Expre- | Sense | 11 | ENS | CD | 1009 | cadherin 11, type 2, OB- | Chr 16 | Reverse | 23 |
| RC.58_ | sion | (Fully | | G000 | H1 | | cadherin (osteoblast) | | Strand | |
| s_at | probe | Exonic) | | 0014 | 1 | | [Source: HGNC | | | |
| | set | | | 0937 | | | Symbol; Acc: 1750] | | | |
| OCMX. | Expre- | Sense | 11 | ENS | CD | 1009 | cadherin 11, type 2, OB- | Chr 16 | Reverse | 24 |
| 1718.0 | sion | (Fully | | G000 | H1 | | cadherin (osteoblast) | | Strand | |
| 1_s_at | probe | Exonic) | | 0014 | 1 | | [Source: HGNC | | | |
| | set | | | 0937 | | | Symbol; Acc: 1750] | | | |
| OCAD | Expre- | Sense | 11 | ENS | CD | 1009 | cadherin 11, type 2, OB- | Chr 16 | Reverse | 25 |
| A.8067 | sion | (includes | | G000 | H1 | | cadherin (osteoblast) | | Strand | |
| _x_at | probe | Intronic) | | 0014 | 1 | | [Source: HGNC | | | |
| | set | | | 0937 | | | Symbol; Acc: 1750] | | | |
| OCRS. | Expre- | Sense | 11 | ENS | CO | 1300 | collagen, type X, alpha 1 | Chr 6 | Reverse | 26 |
| 383_s_ | sion | (Fully | | G000 | L10 | | [Source: HGNC | | Strand | |
| at | probe | Exonic) | | 0012 | A1 | | Symbol; Acc: 2185] | | | |
| | set | | | 3500 | | | | | | |
| OC3SN | Expre- | Sense | 11 | ENS | CO | 1300 | collagen, type X, alpha 1 | Chr 6 | Reverse | 27 |
| G.1834- | sion | (Fully | | G000 | L10 | | [Source: HGNC | | Strand | |
| 947a_ | probe | Exonic) | | 0012 | Al | | Symbol; Acc: 2185] | | | |
| s_at | set | | | 3500 | | | | | | |
| OC3P. | Expre- | Sense | 11 | ENS | CO | 1301 | collagen, type XI, alpha 1 | Chr 1 | Reverse | 28 |
| 1561.0 | sion | (Fully | | G000 | L11 | | [Source: HGNC | | Strand | |
| 1_s_at | probe | Exonic) | | 0006 | A1 | | Symbol; Acc: 2186] | | | |
| | set | | | 0718 | | | | | | |
| OC3P. | Expre- | Sense | 11 | ENS | CO | 1301 | collagen, type XI, alpha 1 | Chr 1 | Reverse | 29 |
| 6907.0 | sion | (Fully | | G000 | L11 | | [Source: HGNC | | Strand | |
| 1_s_at | probe | Exonic) | | 0006 | Al | | Symbol; Acc: 2186] | | | |
| | set | | | 0718 | | | | | | |
| OC3P. | Expre- | Sense | 3 | ENS | CO | 1301 | collagen, type XI, alpha 1 | Chr 1 | Reverse | 30 |
| 1561.0 | sion | (Fully | | G000 | L11 | | [Source: HGNC | | Strand | |
| 1_x_at | probe | Exonic) | | 0006 | Al | | Symbol; Acc: 2186] | | | |
| | set | | | 0718 | | | | | | |
| OCAD | Expre- | Sense | 11 | ENS | CO | 1301 | collagen, type XI, alpha 1 | Chr 1 | Reverse | 31 |
| A.4133_ | sion | (includes | | G000 | L11 | | [Source: HGNC | | Strand | |
| s_at | probe | Intronic) | | 0006 | A1 | | Symbol; Acc: 2186] | | | |
| | set | | | 0718 | | | | | | |
| OC3SN | Expre- | Sense | 11 | ENS | CO | 1301 | collagen, type XI, alpha 1 | Chr 1 | Reverse | 32 |
| Gnh.16 | sion | (includes | | G000 | L11 | | [Source: HGNC | | Strand | |
| 343_x_ | probe | Intronic) | | 0006 | Al | | Symbol; Acc: 2186] | | | |
| at | set | | | 0718 | | | | | | |
| OC3SN | Expre- | Sense | 9 | ENS | CO | 1278 | collagen, type I, alpha 2 | Chr 7 | Forward | 33 |
| Gn.847 | sion | (Fully | | G000 | L1A | | [Source: HGNC | | Strand | |
| 4- | probe | Exonic) | | 0016 | 2 | | Symbol; Acc: 2198] | | | |
| 50a_x_ | set | | | 4692 | | | | | | |
| at | | | | | | | | | | |
| OCMX. | Expre- | Sense | 11 | ENS | CO | 1278 | collagen, type I, alpha 2 | Chr 7 | Forward | 34 |
| 184.C1 | sion | (Fully | | G000 | L1A | | [Source: HGNC | | Strand | |
| 1_s_at | probe | Exonic) | | 0016 | 2 | | Symbol; Acc: 2198] | | | |
| | set | | | 4692 | | | | | | |
| OC3SN | Expre- | Sense | 9 | ENS | CO | 1278 | collagen, type I, alpha 2 | Chr 7 | Forward | 35 |
| G.115- | sion | (includes | | G000 | L1A | | [Source: HGNC | | Strand | |
| 2502a_ | probe | Intronic) | | 0016 | 2 | | Symbol; Acc: 2198] | | | |
| at | set | | | 4692 | | | | | | |
| OC3SN | Expre- | Sense | 11 | ENS | CO | 1278 | collagen, type I, alpha 2 | Chr 7 | Forward | 36 |
| G.116- | sion | (Fully | | G000 | L1A | | [Source: HGNC | | Strand | |
| 9169a_ | probe | Exonic) | | 0016 | 2 | | Symbol; Acc: 2198] | | | |
| s_at | set | | | 4692 | | | | | | |
| OC3P. | Expre- | Sense | 1 | ENS | CO | 1278 | collagen, type I, alpha 2 | Chr 7 | Forward | 37 |
| 60.CB | sion | (Fully | | G000 | L1A | | [Source: HGNC | | Strand | |
| 2_x_at | probe | Exonic) | | 0016 | 2 | | Symbol; Acc: 2198] | | | |
| | set | | | 4692 | | | | | | |
| OC3P. | Expre- | Sense | 11 | ENS | CO | 1278 | collagen, type I, alpha 2 | Chr 7 | Forward | 38 |
| 6454.0 | sion | (Fully | | G000 | L1A | | [Source: HGNC | | Strand | |
| 1_s_at | probe | Exonic) | | 0016 | 2 | | Symbol; Acc: 2198] | | | |
| | set | | | 4692 | | | | | | |
| OC3SN | Expre- | Sense | 10 | ENS | CO | 1278 | collagen, type I, alpha 2 | Chr 7 | Forward | 39 |
| G.115- | sion | (includes | | G000 | L1A | | [Source: HGNC | | Strand | |
| 2502a_ | probe | Intronic) | | 0016 | 2 | | Symbol; Acc: 2198] | | | |
| x_at | set | | | 4692 | | | | | | |
| OCMX. | Expre- | Sense | 2 | ENS | CO | 1278 | collagen, type I, alpha 2 | Chr 7 | Forward | 40 |
| 184.C1 | sion | (Fully | | G000 | L1A | | [Source: HGNC | | Strand | |
| 6_x_at | probe | Exonic) | | 0016 | 2 | | Symbol; Acc: 2198] | | | |
| | set | | | 4692 | | | | | | |
| OCHP. | Expre- | Sense | 11 | ENS | CO | 1278 | collagen, type I, alpha 2 | Chr 7 | Forward | 41 |
| 173_x_ | sion | (Fully | | G000 | L1A | | [Source: HGNC | | Strand | |
| at | probe | Exonic) | | 0016 | 2 | | Symbol; Acc: 2198] | | | |
| | set | | | 4692 | | | | | | |
| OC3P. | Expre- | Sense | 1 | ENS | CO | 1278 | collagen, type I, alpha 2 | Chr 7 | Forward | 42 |
| 60.CB | sion | (Fully | | G000 | L1A | | [Source: HGNC | | Strand | |
| 1_x_at | probe | Exonic) | | 0016 | 2 | | Symbol; Acc: 2198] | | | |
| | set | | | 4692 | | | | | | |
| OC3SN | Expre- | Sense | 11 | ENS | CO | 1278 | collagen, type I, alpha 2 | Chr 7 | Forward | 43 |
| Gn.253 | sion | (Fully | | G000 | L1A | | [Source: HGNC | | Strand | |
| 8- | probe | Exonic) | | 0016 | 2 | | Symbol; Acc: 2198] | | | |
| 539a_x | set | | | 4692 | | | | | | |
| at | | | | | | | | | | |
| OCMX. | Expre- | Sense | 11 | ENS | CO | 1278 | collagen, type I, alpha 2 | Chr 7 | Forward | 44 |
| 184.C1 | sion | (Fully | | G000 | L1A | | [Source: HGNC | | Strand | |
| 6_s_at | probe | Exonic) | | 0016 | 2 | | Symbol; Acc: 2198] | | | |
| | set | | | 4692 | | | | | | |
| OCAD | Expre- | Sense | 11 | ENS | CO | 1281 | collagen, type III, alpha 1 | Chr 2 | Forward | 45 |
| NP.404 | sion | (includes | | G000 | L3A | | [Source: HGNC | | Strand | |
| 8_s_at | probe | Intronic) | | 0016 | 1 | | Symbol; Acc: 2201] | | | |
| | set | | | 8542 | | | | | | |
| OC3P. | Expre- | Sense | 11 | ENS | CO | 1281 | collagen, type III, alpha 1 | Chr 2 | Forward | 46 |
| 81.CB | sion | (Fully | | G000 | L3A | | [Source: HGNC | | Strand | |
| 2_s_at | probe | Exonic) | | 0016 | 1 | | Symbol; Acc: 2201] | | | |
| | set | | | 8542 | | | | | | |
| OC3SN | Expre- | Sense | 11 | ENS | CO | 1281 | collagen, type III, alpha 1 | Chr 2 | Forward | 47 |
| Gnh.19 | sion | (Fully | | G000 | L3A | | [Source: HGNC | | Strand | |
| 127_s_ | probe | Exonic) | | 0016 | 1 | | Symbol; Acc: 2201] | | | |
| at | set | | | 8542 | | | | | | |
| OC3SN | Expre- | Sense | 11 | ENS | CO | 1281 | collagen, type III, alpha 1 | Chr 2 | Forward | 48 |
| Gn.121 | sion | (Fully | | G000 | L3A | | [Source: HGNC | | Strand | |
| 1- | probe | Exonic) | | 0016 | 1 | | Symbol; Acc: 2201] | | | |
| 6a_s_at | set | | | 8542 | | | | | | |
| OCMX. | Expre- | Sense | 11 | ENS | CO | 1290 | collagen, type V, alpha 2 | Chr 2 | Reverse | 49 |
| 338.C1 | sion | (includes | | G000 | L5A | | [Source: HGNC | | Strand | |
| _at | probe | Intronic) | | 0020 | 2 | | Symbol; Acc: 2210] | | | |
| | set | | | 4262 | | | | | | |
| OC3P. | Expre- | Sense | 11 | ENS | CO | 1290 | collagen, type V, alpha 2 | Chr 2 | Reverse | 50 |
| 6029.0 | sion | (Fully | | G000 | L5A | | [Source: HGNC | | Strand | |
| 1_s_at | probe | Exonic) | | 0020 | 2 | | Symbol; Acc: 2210] | | | |
| | set | | | 4262 | | | | | | |
| OCRS | Expre- | Sense | 11 | ENS | CO | 1290 | collagen, type V, alpha 2 | Chr 2 | Reverse | 51 |
| 2.8960_ | sion | (Fully | | G000 | L5A | | [Source: HGNC | | Strand | |
| s_at | probe | Exonic) | | 0020 | 2 | | Symbol; Acc: 2210] | | | |
| | set | | | 4262 | | | | | | |
| OCMX. | Expre- | Sense | 11 | ENS | CO | 1290 | collagen, type V, alpha 2 | Chr 2 | Reverse | 52 |
| 338.C1_ | sion | (includes | | G000 | L5A | | [Source: HGNC | | Strand | |
| x_at | probe | Intronic) | | 0020 | 2 | | Symbol; Acc: 2210] | | | |
| | set | | | 4262 | | | | | | |
| OC3P. | Expre- | Sense | 11 | ENS | CO | 1290 | collagen, type V, alpha 2 | Chr 2 | Reverse | 53 |
| 2713.C | sion | (Fully | | G000 | L5A | | [Source: HGNC | | Strand | |
| 1_s_at | probe | Exonic) | | 0020 | 2 | | Symbol; Acc: 2210] | | | |
| | set | | | 4262 | | | | | | |
| OC3P. | Expre- | Sense | 11 | ENS | CO | 1290 | collagen, type V, alpha 2 | Chr 2 | Reverse | 54 |
| 12307. | sion | (includes | | G000 | L5A | | [Source: HGNC | | Strand | |
| C1_x_ | probe | Intronic) | | 0020 | 2 | | Symbol; Acc: 2210] | | | |
| at | set | | | 4262 | | | | | | |
| OC3SN | Expre- | Sense | 8 | ENS | CO | 1295 | collagen, type VIII, alpha 1 | Chr 3 | Forward | 55 |
| Gnh.18 | sion | (includes | | G000 | L8A | | [Source: HGNC | | Strand | |
| 844_at | probe | Intronic) | | 0014 | 1 | | Symbol; Acc: 2215] | | | |
| | set | | | 4810 | | | | | | |
| OC3P. | Expre- | Sense | 11 | ENS | CO | 1295 | collagen, type VIII, alpha 1 | Chr 3 | Forward | 56 |
| 1087.C | sion | (Fully | | G000 | L8A | | [Source: HGNC | | Strand | |
| 1_s_at | probe | Exonic) | | 0014 | 1 | | Symbol; Acc: 2215] | | | |
| | set | | | 4810 | | | | | | |
| OC3P. | Expre- | Sense | 11 | ENS | CO | 1295 | collagen, type VIII, alpha 1 | Chr 3 | Forward | 57 |
| 13652. | sion | (Fully | | G000 | L8A | | [Source: HGNC | | Strand | |
| C1_s_at | probe | Exonic) | | 0014 | 1 | | Symbol; Acc: 2215] | | | |
| | set | | | 4810 | | | | | | |
| OCAD | Expre- | Sense | 11 | ENS | CO | 1295 | collagen, type VIII, alpha 1 | Chr 3 | Forward | 58 |
| NP.149 | sion | (includes | | G000 | L8A | | [Source: HGNC | | Strand | |
| 32_s_at | probe | Intronic) | | 0014 | 1 | | Symbol; Acc: 2215] | | | |
| | set | | | 4810 | | | | | | |
| OC3P. | Expre- | Sense | 11 | ENS | CO | 1295 | collagen, type VIII, alpha 1 | Chr 3 | Forward | 59 |
| 10562. | sion | (Fully | | G000 | L8A | | [Source: HGNC | | Strand | |
| C1_s_at | probe | Exonic) | | 0014 | 1 | | Symbol; Acc: 2215] | | | |
| | set | | | 4810 | | | | | | |
| OC3SN | Expre- | Sense | 11 | ENS | CO | 51226 | coatomer protein complex, | Chr 17 | Reverse | 60 |
| Gnh.20 | sion | (Fully | | G000 | PZ | | subunit zeta 2 [Source: HGNC | | Strand | |
| 566_s_ | probe | Exonic) | | 0000 | 2 | | Symbol; Acc: 19356] | | | |
| at | set | | | 5243 | | | | | | |
| OCAD | Expre- | Sense | 11 | ENS | CO | 51226 | coatomer protein complex, | Chr 17 | Reverse | 61 |
| A.4902 | sion | (Fully | | G000 | PZ | | subunit zeta 2 [Source: HGNC | | Strand | |
| _s_at | probe | Exonic) | | 0000 | 2 | | Symbol; Acc: 19356] | | | |
| | set | | | 5243 | | | | | | |
| OC3P. | Expre- | Sense | 11 | ENS | CT | 1513 | cathepsin K [Source: HGNC | Chr 1 | Reverse | 62 |
| 4572.C | sion | (Fully | | G000 | SK | | Symbol; Acc: 2536] | | Strand | |
| 1_s_at | probe | Exonic | | 0014 | | | | | | |
| | set | | | 3387 | | | | | | |
| OC3SN | Expre- | Sense | 8 | ENS | FA | 2191 | fibroblast activation protein, | Chr 2 | Reverse | 63 |
| Gn.301 | sion | (Fully | | G000 | P | | alpha [Source: HGNC | | Strand | |
| 6- | probe | Exonic) | | 0007 | | | Symbol; Acc: 3590] | | | |
| 7a_s_at | set | | | 8098 | | | | | | |
| OCAD | Expre- | Sense | 7 | ENS | FA | 2191 | fibroblast activation protein, | Chr 2 | Reverse | 64 |
| A.985 | sion | (includes | | G000 | P | | alpha [Source: HGNC | | Strand | |
| 6_x_at | probe | Intronic) | | 0007 | | | Symbol; Acc: 3590] | | | |
| | set | | | 8098 | | | | | | |
| OC3P. | Expre- | Sense | 9 | ENS | FA | 2191 | fibroblast activation protein, | Chr 2 | Reverse | 65 |
| 8736.C | sion | (Fully | | G000 | P | | alpha [Source: HGNC | | Strand | |
| 1_s_at | probe | Exonic) | | 0007 | | | Symbol; Acc: 3590] | | | |
| | set | | | 8098 | | | | | | |
| OC3SN | Expre- | Sense | 11 | ENS | FN | 2335 | fibronectin 1 [Source: HGNC | Chr 2 | Reverse | 66 |
| Gn.639 | sion | (Fully | | G000 | 1 | | Symbol; Acc: 3778] | | Strand | |
| 7- | probe | Exonic) | | 0011 | | | | | | |
| 360a_at | set | | | 5414 | | | | | | |
| OCMX. | Expre- | Sense | 11 | ENS | FN | 2335 | fibronectin 1 [Source: HGNC | Chr 2 | Reverse | 67 |
| 493.C1_ | sion | (Fully | | G000 | 1 | | Symbol; Acc: 3778] | | Strand | |
| s_at | probe | Exonic) | | 0011 | | | | | | |
| | set | | | 5414 | | | | | | |
| OC3SN | Expre- | Sense | 7 | ENS | FN | 2335 | fibronectin 1 [Source: HGNC | Chr 2 | Reverse | 68 |
| Gnh.14 | sion | (includes | | G000 | 1 | | Symbol; Acc: 3778] | | Strand | |
| 004_at | probe | Intronic) | | 0011 | | | | | | |
| | set | | | 5414 | | | | | | |
| OCEM. | Expre- | Sense | 11 | ENS | FN | 2335 | fibronectin 1 [Source: HGNC | Chr 2 | Reverse | 69 |
| 2081_a | sion | (Fully | | G000 | 1 | | Symbol; Acc: 3778] | | Strand | |
| t | probe | Exonic) | | 0011 | | | | | | |
| | set | | | 5414 | | | | | | |
| OCEM. | Expre- | Sense | 11 | ENS | FN | 2335 | fibronectin 1 [Source: HGNC | Chr 2 | Reverse | 70 |
| 958_x_ | sion | (includes | | G000 | 1 | | Symbol; Acc: 3778] | | Strand | |
| at | probe | Intronic) | | 0011 | | | | | | |
| | set | | | 5414 | | | | | | |
| OCAD | Expre- | Sense | 11 | ENS | FN | 2335 | fibronectin 1 [Source: HGNC | Chr 2 | Reverse | 71 |
| A.7873_ | sion | (includes | | G000 | 1 | | Symbol; Acc: 3778] | | Strand | |
| s_at | probe | Intronic) | | 0011 | | | | | | |
| | set | | | 5414 | | | | | | |
| OCHP. | Expre- | Sense | 11 | ENS | FN | 2335 | fibronectin 1 [Source: HGNC | Chr 2 | Reverse | 72 |
| 451_s_ | sion | (Fully | | G000 | 1 | | Symbol; Acc: 3778] | | Strand | |
| at | probe | Exonic) | | 0011 | | | | | | |
| | set | | | 5414 | | | | | | |
| OCEM. | Expre- | Sense | 11 | ENS | FN | 2335 | fibronectin 1 [Source: HGNC | Chr 2 | Reverse | 73 |
| 958_at | sion | (includes | | G000 | 1 | | Symbol; Acc: 3778] | | Strand | |
| | probe | Intronic) | | 0011 | | | | | | |
| | set | | | 5414 | | | | | | |
| OC3SN | Expre- | Sense | 11 | ENS | FN | 2335 | fibronectin 1 [Source: HGNC | Chr 2 | Reverse | 74 |
| Gn.465 | sion | (includes | | G000 | 1 | | Symbol; Acc: 3778] | | Strand | |
| 0- | probe | Intronic) | | 0011 | | | | | | |
| 857a_x | set | | | 5414 | | | | | | |
| _at | | | | | | | | | | |
| OC3SN | Expre- | Sense | 9 | ENS | FN | 2335 | fibronectin 1 [Source: HGNC | Chr 2 | Reverse | 75 |
| Gnh.59 | sion | (includes | | G000 | 1 | | Symbol; Acc: 3778] | | Strand | |
| 67_at | probe | Intronic) | | 0011 | | | | | | |
| | set | | | 5414 | | | | | | |
| OCEM. | Expre- | Sense | 11 | ENS | FN | 2335 | fibronectin 1 [Source: HGNC | Chr 2 | Reverse | 76 |
| 2082_ | sion | (Fully | | G000 | 1 | | Symbol; Acc: 3778] | | Strand | |
| s_at | probe | Exonic) | | 0011 | | | | | | |
| | set | | | 5414 | | | | | | |
| OCAD | Expre- | Sense | 11 | ENS | FN | 2335 | fibronectin 1 [Source: HGNC | Chr 2 | Reverse | 77 |
| A.1039_ | sion | (Fully | | G000 | 1 | | Symbol; Acc: 3778] | | Strand | |
| s_at | probe | Exonic) | | 0011 | | | | | | |
| | set | | | 5414 | | | | | | |
| OCEM. | Expre- | Sense | 11 | ENS | FN | 2335 | fibronectin 1 [Source: HGNC | Chr 2 | Reverse | 78 |
| 2082_ | sion | (Fully | | G000 | 1 | | Symbol; Acc: 3778] | | Strand | |
| at | probe | Exonic) | | 0011 | | | | | | |
| | set | | | 5414 | | | | | | |
| OC3SN | Expre- | Sense | 8 | ENS | FN | 2335 | fibronectin 1 [Source: HGNC | Chr 2 | Reverse | 79 |
| Gnh.40 | sion | (includes | | G000 | 1 | | Symbol; Acc: 3778] | | Strand | |
| 44_x_a | probe | Intronic) | | 0011 | | | | | | |
| t | set | | | 5414 | | | | | | |
| OC3P. | Expre- | Sense | 11 | ENS | FN | 2335 | fibronectin 1 [Source: HGNC | Chr 2 | Reverse | 80 |
| 843.CB | sion | (Fully | | G000 | 1 | | Symbol; Acc: 3778] | | Strand | |
| 1- | probe | Exonic) | | 0011 | | | | | | |
| 415a_s | set | | | 5414 | | | | | | |
| at | | | | | | | | | | |
| OCHP. | Expre- | Sense | 11 | ENS | FN | 2335 | fibronectin 1 [Source: HGNC | Chr 2 | Reverse | 81 |
| 470_s_ | sion | (Fully | | G000 | 1 | | Symbol; Acc: 3778] | | Strand | |
| at | probe | Exonic) | | 0011 | | | | | | |
| | set | | | 5414 | | | | | | |
| OC3SN | Expre- | Sense | 8 | ENS | FN | 2335 | fibronectin 1 [Source: HGNC | Chr 2 | Reverse | 82 |
| Gnh.14 | sion | (includes | | G000 | 1 | | Symbol; Acc: 3778] | | Strand | |
| 004_x_ | probe | Intronic) | | 0011 | | | | | | |
| at | set | | | 5414 | | | | | | |
| OCEM. | Expre- | Sense | 11 | ENS | FN | 2335 | fibronectin 1 [Source: HGNC | Chr 2 | Reverse | 83 |
| 2081_ | sion | (Fully | | G000 | 1 | | Symbol; Acc: 3778] | | Strand | |
| x_at | probe | Exonic) | | 0011 | | | | | | |
| | set | | | 5414 | | | | | | |
| OC3SN | Expre- | Sense | 11 | ENS | FN | 2335 | fibronectin 1 [Source: HGNC | Chr 2 | Reverse | 84 |
| Gnh.92 | sion | (includes | | G000 | 1 | | Symbol; Acc: 3778] | | Strand | |
| 61_at | probe | Intronic) | | 0011 | | | | | | |
| | set | | | 5414 | | | | | | |
| OC3P. | Expre- | Sense | 8 | ENS | FZ | 8321 | frizzled family receptor 1 | Chr 7 | Forward | 85 |
| 4921.C | sion | (Fully | | G000 | D1 | | [Source: HGNC | | Strand | |
| 1_at | probe | Exonic) | | 0015 | | | Symbol; Acc: 4038] | | | |
| | set | | | 7240 | | | | | | |
| OC3P. | Expre- | Sense | 11 | ENS | FZ | 8321 | frizzled family receptor 1 | Chr 7 | Forward | 86 |
| 4921.C | sion | (Fully | | G000 | D1 | | [Source: HGNC | | Strand | |
| 1- | probe | Exonic) | | 0015 | | | Symbol; Acc: 4038] | | | |
| 347a_s | set | | | 7240 | | | | | | |
| at | | | | | | | | | | |
| OCAD | Expre- | Sense | 11 | ENS | FZ | 8321 | frizzled family receptor 1 | Chr 7 | Forward | 87 |
| NP.757 | sion | (Fully | | G000 | D1 | | [Source: HGNC | | Strand | |
| 9_s_at | probe | Exonic) | | 0015 | | | Symbol; Acc: 4038] | | | |
| | set | | | 7240 | | | | | | |
| OC3P. | Expre- | Sense | 8 | ENS | FZ | 8321 | frizzled family receptor 1 | Chr 7 | Forward | 88 |
| 4921.C | sion | (Fully | | G000 | D1 | | [Source: HGNC | | Strand | |
| 1_x_at | probe | Exonic) | | 0015 | | | Symbol; Acc: 4038] | | | |
| | set | | | 7240 | | | | | | |
| OCAD | Expre- | Sense | 11 | ENS | GF | 9945 | glutamine-fructose-6-phosphate | Chr 5 | Reverse | 89 |
| A.1231 | sion | (includes | | G000 | PT | | transaminase 2 [Source: HGNC | | Strand | |
| 9_s_at | probe | Intronic) | | 0013 | 2 | | Symbol; Acc: 4242] | | | |
| | set | | | 1459 | | | | | | |
| OC3SN | Expre- | Sense | 10 | ENS | GF | 9945 | glutamine-fructose-6-phosphate | Chr 5 | Reverse | 90 |
| Gnh.16 | sion | (includes | | G000 | PT | | transaminase 2 [Source: HGNC | | Strand | |
| 386_at | probe | Intronic) | | 0013 | 2 | | Symbol; Acc: 4242] | | | |
| | set | | | 1459 | | | | | | |
| OCHP. | Expre- | Sense | 11 | ENS | OF | 9945 | glutamine-fructose-6-phosphate | Chr 5 | Reverse | 91 |
| 202_s_ | sion | (Fully | | G000 | PT | | transaminase 2 [Source: HGNC | | Strand | |
| at | probe | Exonic) | | 0013 | 2 | | Symbol; Acc: 4242] | | | |
| | set | | | 1459 | | | | | | |
| OCHP. | Expre- | Sense | 11 | ENS | GJ | 2706 | gap junction protein, beta 2, | Chr 13 | Reverse | 92 |
| 838_s_ | sion | (Fully | | G000 | B2 | | 26 kDa [Source: HGNC | | Strand | |
| at | probe | Exonic) | | 0016 | | | Symbol; Acc: 4284] | | | |
| | set | | | 5474 | | | | | | |
| OC3P. | Expre- | Sense | 11 | ENS | GJ | 2706 | gap junction protein, beta 2, | Chr 13 | Reverse | 93 |
| 7485.C | sion | (Fully | | G000 | B2 | | 26 kDa [Source: HGNC | | Strand | |
| 1- | probe | Exonic) | | 0016 | | | Symbol; Acc: 4284] | | | |
| 335a_ | set | | | 5474 | | | | | | |
| s_at | | | | | | | | | | |
| OC3SN | Expre- | Sense | 9 | ENS | IGF | 147920 | IGF-like family member 2 | Chr 19 | Forward | 94 |
| Gnh.14 | sion | (Fully | | G000 | L2 | | [Source: HGNC | | Strand | |
| 657_s_ | probe | Exonic) | | 0020 | | | Symbol; Acc: 32929] | | | |
| at | set | | | 4866 | | | | | | |
| ADXBa | Almac | Sense | 1 | ENS | IGF | 147920 | IGF-like family member 2 | Chr 19 | Forward | N/A |
| d28_at | pixe- | (Fully | | G000 | L2 | | [Source: HGNC | | Strand | |
| | llation | Exonic) | | 0020 | | | Symbol; Acc: 32929] | | | |
| | control | | | 4866 | | | | | | |
| ADXBa | Almac | Sense | 1 | ENS | EX | 101929 | EXTL3 antisense RNA 1 | Chr 8 | Reverse | N/A |
| d28_at | pixe- | (Fully | | G000 | TL3- | 402 | [Source: HGNC | | Strand | |
| | tion | Exonic) | | 0024 | AS | | Symbol; Acc: 27985] | | | |
| | control | | | 6339 | 1 | | | | | |
| ADXBa | Almac | Sense | 1 | ENS | N/A | N/A | NOVEL lincRNA | Chr 2 | Reverse | N/A |
| d28_at | pixe- | (Fully | | G000 | | | (Clone_based_vega_gene) | | Strand | |
| | llation | Exonic) | | 0027 | | | | | | |
| | control | | | 2994 | | | | | | |
| ADXBa | Almac | Sense | 1 | ENS | N/A | N/A | NOVEL sense_oyerlapping | Chr 2 | Forward | N/A |
| d28_at | pixe- | (Fully | | G000 | | | (Clone_based_vega_gene) | | Strand | |
| | llation | Exonic) | | 0026 | | | | | | |
| | control | | | 1829 | | | | | | |
| ADXBa | Almac | Sense | 1 | ENS | ABI | 25890 | ABI family, member 3 (NESH) | Chr 3 | Reverse | N/A |
| d28_at | pixe- | (Fully | | G000 | 3B | | binding protein [Source: HGNC | | Strand | |
| | llation | Exonic) | | 0015 | P | | Symbol; Acc: 17265] | | | |
| | control | | | 4175 | | | | | | |
| ADXBa | Almac | Sense | 1 | ENS | IGF | 147920 | IGF-like family member 2 | Chr 19 | Forward | N/A |
| d38_at | pixe- | (Fully | | G000 | L2 | | [Source: HGNC | | Strand | |
| | llation | Exonic) | | 0020 | | | Symbol; Acc: 32929] | | | |
| | control | | | 4866 | | | | | | |
| ADXBa | Almac | Sense | 1 | ENS | KC | 3759 | potassium inwardly-rectifying | Chr 17 | Forward | N/A |
| d38_at | pixe- | (Fully | | G000 | NJ2 | | channel, subfamily J, member 2 | | Strand | |
| | llation | Exonic) | | 0012 | | | [Source: HGNC | | | |
| | control | | | 3700 | | | Symbol; Acc: 6263] | | | |
| ADXBa | Almac | Sense | 1 | ENS | IL1 | 27189 | interleukin 17C [Source: | Chr 16 | Forward | N/A |
| d38_at | pixe- | (Fully | | G000 | 7C | | HGNC Symbol; Acc: 5983] | | Strand | |
| | llation | Exonic) | | 0012 | | | | | | |
| | control | | | 4391 | | | | | | |
| ADXBa | Almac | Sense | 1 | ENS | EX | 23086 | exophilin 5 [Source: HGNC | Chr 11 | Reverse | N/A |
| d38_at | pixe- | (Fully | | G000 | PH | | Symbol; Acc: 30578] | | Strand | |
| | llation | Exonic) | | 0011 | 5 | | Stra | | | |
| | control | | | 0723 | | | | | | |
| ADXBa | Almac | Sense | 1 | ENS | GP | 11318 | G protein-coupled receptor 182 | Chr 12 | Forward | N/A |
| d38_at | pixe- | (Fully | | G000 | R1 | | [Source: HGNC | | Strand | |
| | llation | Exonic) | | 0016 | 82 | | Symbol; Acc: 13708] | | | |
| | control | | | 6856 | | | | | | |
| ADXBa | Almac | Sense | 1 | ENS | GS | 2949 | glutathione S-transferase mu 5 | Chr 1 | Forward | N/A |
| d38_at | pixe- | (Fully | | G000 | TM | | [Source: HGNC | | Strand | |
| | llation | Exonic) | | 0013 | 5 | | Symbol; Acc: 4637] | | | |
| | control | | | 4201 | | | | | | |
| ADXBa | Almac | Sense | 1 | ENS | SL | 11136 | solute carrier family 7 (amino | Chr 19 | Reverse | N/A |
| d38_at | pixe- | (Fully | | G000 | C7 | | acid transporter light chain, | | Strand | |
| | llation | Exonic) | | 0002 | A9 | | bo, +system), member 9 | | | |
| | control | | | 1488 | | | [Source: HGNC | | | |
| | | | | | | | Symbol; Acc: 11067] | | | |
| ADXBa | Almac | Sense | 1 | ENS | OR | 26585 | gremlin 1, DAN family BMP | Chr 15 | Forward | N/A |
| d38_at | pixe- | (Fully | | G000 | EM | | antagonist [Source: HGNC | | Strand | |
| | llation | Exonic) | | 0016 | 1 | | Symbol; Acc: 2001] | | | |
| | control | | | 6923 | | | | | | |
| OC3SN | Expre- | Sense | 11 | ENS | INH | 3624 | inhibin, beta A [Source: HGNC | Chr 7 | Reverse | 95 |
| Gnh.36 | sion | (Fully | | G000 | BA | | Symbol; Acc: 6066] | | Strand | |
| 06_s_ | probe | Exonic) | | 0012 | | | | | | |
| at | set | | | 2641 | | | | | | |
| OCEM. | Expre- | Sense | 11 | ENS | INH | 3624 | inhibin, beta A [Source: HGNC | Chr 7 | Reverse | 96 |
| 2109_ | sion | (Fully | | G000 | BA | | Symbol; Acc: 6066] | | Strand | |
| s_at | probe | Exonic) | | 0012 | | | | | | |
| | set | | | 2641 | | | | | | |
| OCEM. | Expre- | Sense | 11 | ENS | INH | 3624 | inhibin, beta A [Source: HGNC | Chr 7 | Reverse | 97 |
| 2108_ | sion | (Fully | | G000 | BA | | Symbol; Acc: 6066] | | Strand | |
| at | probe | Exonic) | | 0012 | | | | | | |
| | set | | | 2641 | | | | | | |
| OCRS. | Expre- | Sense | 11 | ENS | INH | 3624 | inhibin, beta A [Source: HGNC | Chr 7 | Reverse | 98 |
| 977_s_ | sion | (Fully | | G000 | BA | | Symbol; Acc: 6066] | | Strand | |
| at | probe | Exonic) | | 0012 | | | | | | |
| | set | | | 2641 | | | | | | |
| OCEM. | Expre- | Sense | 8 | ENS | INH | 3624 | inhibin, beta A [Source: HGNC | Chr 7 | Reverse | 99 |
| 2109_ | sion | (Fully | | G000 | BA | | Symbol; Acc: 6066] | | Strand | |
| at | probe | Exonic) | | 0012 | | | | | | |
| | set | | | 2641 | | | | | | |
| OC3P. | Expre- | Sense | 11 | ENS | INH | 3624 | inhibin, beta A [Source: HGNC | Chr 7 | Reverse | 100 |
| 10944. | sion | (Fully | | G000 | BA | | Symbol; Acc: 6066] | | Strand | |
| C1_s_ | probe | Exonic) | | 0012 | | | | | | |
| at | set | | | 2641 | | | | | | |
| OCAD | Expre- | Sense | 11 | ENS | INH | 3624 | inhibin, beta A [Source: HGNC | Chr 7 | Reverse | 101 |
| NP.761 | sion | (Fully | | G000 | BA | | Symbol; Acc: 6066] | | Strand | |
| 8_s_at | probe | Exonic) | | 0012 | | | | | | |
| | set | | | 2641 | | | | | | |
| OCEM. | Expre- | Sense | 11 | ENS | INH | 3624 | inhibin, beta A [Source: HGNC | Chr 7 | Reverse | 102 |
| 2108_ | sion | (Fully | | G000 | BA | | Symbol; Acc: 6066] | | Strand | |
| x_at | probe | Exonic) | | 0012 | | | | | | |
| | set | | | 2641 | | | | | | |
| OC3P. | Expre- | Sense | 11 | ENS | ITG | 3678 | integrin, alpha 5 (fibronectin | Chr 12 | Reverse | 103 |
| 2699.0 | sion | (Fully | | G000 | A5 | | receptor, alpha polypeptide) | | Strand | |
| 1_s_at | probe | Exonic) | | 0016 | | | [Source: HGNC | | | |
| | set | | | 1638 | | | Symbol; Acc: 6141] | | | |
| OC3SN | Expre- | Sense | 11 | ENS | KIF | 55083 | kinesin family member 26B | Chr 1 | Forward | 104 |
| Gnh.12 | sion | (includes | | G000 | 26B | | [Source: HGNC | | Strand | |
| 739_x_ | probe | Intronic) | | 0016 | | | Symbol; Acc: 25484] | | | |
| at | set | | | 2849 | | | | | | |
| OC3SN | Expre- | Sense | 11 | ENS | KIF | 55083 | kinesin family member 26B | Chr 1 | Forward | 105 |
| Gnh.12 | sion | (includes | | G000 | 26B | | [Source: HGNC | | Strand | |
| 739_at | probe | Intronic) | | 0016 | | | Symbol; Acc: 25484] | | | |
| | set | | | 2849 | | | | | | |
| OC3SN | Expre- | Sense | 9 | ENS | KIF | 55083 | kinesin family member 26B | Chr 1 | Forward | 106 |
| Gnh.31 | sion | (includes | | G000 | 26B | | [Source: HGNC | | Strand | |
| 11_x_ | probe | Intronic) | | 0016 | | | Symbol; Acc: 25484] | | | |
| at | set | | | 2849 | | | | | | |
| OCAD | Expre- | Sense | 10 | ENS | KIF | 55083 | kinesin family member 26B | Chr 1 | Forward | 107 |
| NP.177 | sion | (Fully | | G000 | 26B | | [Source: HGNC | | Strand | |
| 12_s_ | probe | Exonic) | | 0016 | | | Symbol; Acc: 25484] | | | |
| at | set | | | 2849 | | | | | | |
| OCAD | Expre- | Sense | 11 | ENS | KIF | 55083 | kinesin family member 26B | Chr 1 | Forward | 108 |
| A.1652 | sion | (includes | | G000 | 26B | | [Source: HGNC | | Strand | |
| _x_at | probe | Intronic) | | 0016 | | | Symbol; Acc: 25484] | | | |
| | set | | | 2849 | | | | | | |
| OC3SN | Expre- | Sense | 11 | ENS | KIF | 55083 | kinesin family member 26B | Chr 1 | Forward | 109 |
| Gnh.26 | sion | (includes | | G000 | 26B | | [Source: HGNC | | Strand | |
| 7_s_at | probe | Intronic) | | 0016 | | | Symbol; Acc: 25484] | | | |
| | set | | | 2849 | | | | | | |
| OC3SN | Expre- | Sense | 11 | ENS | KIF | 55083 | kinesin family member 26B | Chr 1 | Forward | 110 |
| Gn.747 | sion | (Fully | | G000 | 26B | | [Source: HGNC | | Strand | |
| 1- | probe | Exonic) | | 0016 | | | Symbol; Acc: 25484] | | | |
| 125a_ | set | | | 2849 | | | | | | |
| s_at | | | | | | | | | | |
| OC3SN | Expre- | Sense | 11 | ENS | KIF | 55083 | kinesin family member 26B | Chr 1 | Forward | 111 |
| Gnh.96 | sion | (includes | | G000 | 26B | | [Source: HGNC | | Strand | |
| 22_x_ | probe | Intronic) | | 0016 | | | Symbol; Acc: 25484] | | | |
| at | set | | | 2849 | | | | | | |
| OC3SN | Expre- | Sense | 11 | ENS | KIF | 55083 | kinesin family member 26B | Chr 1 | Forward | 112 |
| Gnh.16 | sion | (includes | | G000 | 26B | | [Source: HGNC | | Strand | |
| 827_s_ | probe | Intronic) | | 0016 | | | Symbol; Acc: 25484] | | | |
| at | set | | | 2849 | | | | | | |
| OCHP. | Expre- | Sense | 11 | ENS | LO | 4016 | lysyl oxidase-like 1 | Chr 15 | Reverse | 113 |
| 1306_ | sion | (Fully | | G000 | XL1 | | [Source: HGNC | | Strand | |
| s_at | probe | Exonic) | | 0012 | | | Symbol; Acc: 6665] | | | |
| | set | | | 9038 | | | | | | |
| OCHP. | Expre- | Sense | 11 | ENS | LU | 4060 | lumican [Source: HGNC | Chr 12 | Reverse | 114 |
| 1534_ | sion | (Fully | | G000 | M | | Symbol; Acc: 6724] | | Strand | |
| x_at | probe | Exonic) | | 0013 | | | | | | |
| | set | | | 9329 | | | | | | |
| OCHP. | Expre- | Sense | 11 | ENS | LU | 4060 | lumican [Source: HGNC | Chr 12 | Reverse | 115 |
| 1534_ | sion | (Fully | | G000 | M | | Symbol; Acc: 6724] | | Strand | |
| s_at | probe | Exonic) | | 0013 | | | | | | |
| | set | | | 9329 | | | | | | |
| OC3SN | Expre- | Sense | 11 | ENS | MI | 100302 | microRNA 1245a | Chr 2 | Reverse | 116 |
| Gnh.34 | sion | (Fully | | G000 | R1 | 219/// | [Source: HGNC | | Strand | |
| 22_s_ | probe | Exonic) | | 0022 | 245 | 100616 | Symbol; Acc: 35311] | | | |
| at | set | | | 1502 | A | 324 | | | | |
| OCHP. | Expre- | Sense | 11 | ENS | MM | 4322 | matrix metallopeptidase 13 | Chr 11 | Reverse | 117 |
| 983_s_ | sion | (Fully | | G000 | P13 | | (collagenase 3) [Source: HGNC | | Strand | |
| at | probe | Exonic) | | 0013 | | | Symbol; Acc: 7159] | | | |
| | set | | | 7745 | | | | | | |
| OCHP. | Expre- | Sense | 11 | ENS | MM | 4323 | matrix metallopeptidase 14 | Chr 14 | Forward | 118 |
| 228_s_ | sion | (Fully | | G000 | P14 | | (membrane-inserted) | | Strand | |
| at | probe | Exonic) | | 0015 | | | [Source: HGNC | | | |
| | set | | | 7227 | | | Symbol; Acc: 7160] | | | |
| OC3P. | Expre- | Sense | 9 | ENS | MM | 4323 | matrix metallopeptidase 14 | Chr 14 | Forward | 119 |
| 4123.C | sion | (Fully | | G000 | P14 | | (membrane-inserted) | | Strand | |
| 1_x_at | probe | Exonic) | | 0015 | | | [Source: HGNC | | | |
| | set | | | 7227 | | | Symbol; Acc: 7160] | | | |
| OC3P. | Expre- | Sense | 11 | ENS | MM | 4323 | matrix metallopeptidase 14 | Chr 14 | Forward | 120 |
| 4123.C | sion | (Fully | | G000 | P14 | | (membrane-inserted) | | Strand | |
| 1_s_at | probe | Exonic) | | 0015 | | | [Source: HGNC | | | |
| | set | | | 7227 | | | Symbol; Acc: 7160] | | | |
| OC3P. | Expre- | Sense | 11 | ENS | MM | 4313 | matrix metallopeptidase 2 | Chr 16 | Forward | 121 |
| 1163.C | sion | (Fully | | G000 | P2 | | (gelatinase A, 72 kDa | | Strand | |
| 3_s_at | probe | Exonic) | | 0008 | | | gelatinase, 72 kDa type IV | | | |
| | set | | | 7245 | | | collagenase) [Source: HGNC | | | |
| | | | | | | | Symbol; Acc: 7166] | | | |
| OCHP. | Expre- | Sense | 11 | ENS | MM | 4313 | matrix metallopeptidase 2 | Chr 16 | Forward | 122 |
| 374_s_ | sion | (Fully | | G000 | P2 | | (gelatinase A, 72 kDa | | Strand | |
| at | probe | Exonic) | | 0008 | | | gelatinase, 72 kDa type IV | | | |
| | set | | | 7245 | | | collagenase) [Source: HGNC | | | |
| | | | | | | | Symbol; Acc: 7166] | | | |
| OCAD | Expre- | Sense | 11 | ENS | MM | 4313 | matrix metallopeptidase 2 | Chr 16 | Forward | 123 |
| NP.725 | sion | (Fully | | G000 | P2 | | (gelatinase A, 72 kDa | | Strand | |
| 1_s_at | probe | Exonic) | | 0008 | | | gelatinase, 72 kDa type IV | | | |
| | set | | | 7245 | | | collagenase) [Source: HGNC | | | |
| | | | | | | | Symbol; Acc: 7166] | | | |
| OCAD | Expre- | Sense | 11 | ENS | MM | 4313 | matrix metallopeptidase 2 | Chr 16 | Forward | 124 |
| A.2310_ | sion | (Fully | | G000 | P2 | | (gelatinase A, 72 kDa | | Strand | |
| s_at | probe | Exonic) | | 0008 | | | gelatinase, 72 kDa type IV | | | |
| | set | | | 7245 | | | collagenase) [Source: HGNC | | | |
| | | | | | | | Symbol; Acc: 7166] | | | |
| OCAD | Expre- | Sense | 11 | ENS | MR | 10335 | murine retrovirus integration | Chr 11 | Reverse | 125 |
| A.3580_ | sion | (Fully | | G000 | VI1 | | site 1 homolog [Source: HGNC | | Strand | |
| s_at | probe | Exonic) | | 0007 | | | Symbol; Acc: 7237] | | | |
| | set | | | 2952 | | | | | | |
| OC3P. | Expre- | Sense | 11 | ENS | MR | 10335 | murine retrovirus integration | Chr 11 | Reverse | 126 |
| 1058.0 | sion | (Fully | | G000 | VI1 | | site 1 homolog [Source: HGNC | | Strand | |
| l_s_at | probe | Exonic) | | 0007 | | | Symbol; Acc: 7237] | | | |
| | set | | | 2952 | | | | | | |
| OC3P. | Expre- | Sense | 11 | ENS | MR | 10335 | murine retrovirus integration | Chr 11 | Reverse | 127 |
| 13126. | sion | (Fully | | G000 | VI1 | | site 1 homolog [Source: HGNC | | Strand | |
| C1_s_at | probe | Exonic) | | 0007 | | | Symbol; Acc: 7237] | | | |
| | set | | | 2952 | | | | | | |
| OCAD | Expre- | Sense | 11 | ENS | MR | 10335 | murine retrovirus integration | Chr 11 | Reverse | 128 |
| NP.102 | sion | (Fully | | G000 | VI1 | | site 1 homolog [Source: HGNC | | Strand | |
| 37_s_at | probe | Exonic) | | 0007 | | | Symbol; Acc: 7237] | | | |
| | set | | | 2952 | | | | | | |
| OCAD | Expre- | Sense | 9 | ENS | NK | 85409 | naked cuticle homolog 2 | Chr 5 | Forward | 129 |
| NP.584 | sion | (Fully | | G000 | D2 | | (Drosophila) [Source: HGNC | | Strand | |
| 9_s_at | probe | Exonic) | | 0014 | | | Symbol; Acc: 17046] | | | |
| | set | | | 5506 | | | | | | |
| OCAD | Expre- | Sense | 11 | ENS | NT | 50863 | neurotrimin [Source: HGNC | Chr 11 | Forward | 130 |
| NP.655 | sion | (Fully | | G000 | M | | Symbol; Acc: 17941] | | Strand | |
| 5_s_at | probe | Exonic) | | 0018 | | | | | | |
| | set | | | 2667 | | | | | | |
| OCAD | Expre- | Sense | 11 | ENS | NT | 50863 | neurotrimin [Source: HGNC | Chr 11 | Forward | 131 |
| A.3177_ | sion | (Fully | | G000 | M | | Symbol; Acc: 17941] | | Strand | |
| s_at | probe | Exonic) | | 0018 | | | | | | |
| | set | | | 2667 | | | | | | |
| OC3SN | Expre- | Sense | 11 | ENS | NT | 50863 | neurotrimin [Source: HGNC | Chr 11 | Forward | 132 |
| G.2346- | sion | (Fully | | G000 | M | | Symbol; Acc: 17941] | | Strand | |
| 440a_ | probe | Exonic) | | 0018 | | | | | | |
| s_at | set | | | 2667 | | | | | | |
| OC3SN | Expre- | Sense | 1 | ENS | NT | 50863 | neurotrimin [Source: HGNC | Chr 11 | Forward | 133 |
| Gnh.75 | sion | (includes | | G000 | M | | Symbol; Acc: 17941] | | Strand | |
| 67_x_at | probe | Intronic) | | 0018 | | | | | | |
| | set | | | 2667 | | | | | | |
| OC3SN | Expre- | Sense | 1 | ENS | DC | 196513 | decapping mRNA 1B | Chr 12 | Reverse | 133 |
| Gnh.75 | sion | (includes | | G000 | P1 | | [Source: HGNC | | Strand | |
| 67_x_at | probe | Intronic) | | 0015 | B | | Symbol; Acc: 24451] | | | |
| | set | | | 1065 | | | | | | |
| OCHP. | Expre- | Sense | 11 | ENS | PL | 5328 | plasminogen activator, | Chr 10 | Forward | 134 |
| 739_s_ | sion | (Fully | | G000 | AU | | urokinase [Source: HGNC | | Strand | |
| at | probe | Exonic) | | 0012 | | | Symbol; Acc: 9052] | | | |
| | set | | | 2861 | | | | | | |
| OCAD | Expre- | Sense | 10 | ENS | PL | 5328 | plasminogen activator, | Chr 10 | Forward | 135 |
| NP.865 | sion | (Fully | | G000 | AU | | urokinase [Source: HGNC | | Strand | |
| 3_s_at | probe | Exonic) | | 0012 | | | Symbol; Acc: 9052] | | | |
| | set | | | 2861 | | | | | | |
| ADXGo | Almac | Sense | 1 | ENS | TP | 127262 | tumor protein p63 regulated 1- | Chr 1 | Forward | N/A |
| od72_at | pixe- | (Fully | | G000 | RG | | like [Source: HGNC | | Strand | |
| | llation | Exonic) | | 0015 | 1L | | Symbol; Acc: 27007] | | | |
| | control | | | 8109 | | | | | | |
| ADXGo | Almac | Sense | 1 | ENS | SA | 51128 | SAR1 homolog B (S. | Chr 5 | Reverse | N/A |
| od72_at | pixe- | (Fully | | G000 | R1 | | cerevisiae) [Source: HGNC | | Strand | |
| | llation | Exonic) | | 0015 | B | | Symbol; Acc: 10535] | | | |
| | control | | | 2700 | | | | | | |
| ADXGo | Almac | Sense | 1 | ENS | RN | 388591 | ring finger protein 207 | Chr 1 | Forward | N/A |
| od72_at | pixe- | (Fully | | G000 | F20 | | [Source: HGNC | | Strand | |
| | llation | Exonic) | | 0015 | 7 | | Symbol; Acc: 32947] | | | |
| | control | | | 8286 | | | | | | |
| ADXGo | Almac | Sense | 1 | ENS | UQ | 55245 | ubiquinol-cytochrome c | Chr 20 | Reverse | N/A |
| od72_at | pixe- | (Fully | | G000 | CC | | reductase complex assembly | | Strand | |
| | llation | Exonic) | | 0010 | 1 | | factor 1 [Source: HGNC | | | |
| | control | | | 1019 | | | Symbol; Acc: 15891] | | | |
| ADXGo | Almac | Sense | 1 | ENS | GP | 9289 | G protein-coupled receptor 56 | Chr 16 | Forward | N/A |
| od72_at | pixe- | (Fully | | G000 | R5 | | [Source: HGNC | | Strand | |
| | llation | Exonic) | | 0020 | 6 | | Symbol; Acc: 4512] | | | |
| | control | | | 5336 | | | | | | |
| ADXGo | Almac | Sense | 1 | ENS | PM | 5376 | peripheral myelin protein 22 | Chr 17 | Reverse | N/A |
| od72_at | pixe- | (Fully | | G000 | P22 | | [Source: HGNC | | Strand | |
| | llation | Exonic) | | 0010 | | | Symbol; Acc: 9118] | | | |
| | control | | | 9099 | | | | | | |
| ADXGo | Almac | Sense | 1 | ENS | NU | 83540 | NUF2, NDC80 kinetochore | Chr 1 | Forward | N/A |
| od72_at | pixe- | (Fully | | G000 | F2 | | complex component | | Strand | |
| | llation | Exonic) | | 0014 | | | [Source: HGNC | | | |
| | control | | | 3228 | | | Symbol; Acc: 14621] | | | |
| ADXGo | Almac | Sense | 1 | ENS | API | 378708/// | apoptosis-inducing, TAF9-like | Chr 1 | Forward | N/A |
| od72_at | pixe- | (Fully | | G000 | TD | 100526 | domain 1 [Source: HGNC | | Strand | |
| | llation | Exonic) | | 0017 | 1 | 739 | Symbol; Acc: 23163] | | | |
| | control | | | 5279 | | | | | | |
| ADXGo | Almac | Sense | 1 | ENS | NU | 23636 | nucleoporin 62 kDa | Chr 19 | Reverse | N/A |
| od72_at | pixe- | (Fully | | G000 | P62 | | [Source: HGNC | | Strand | |
| | llation | Exonic) | | 0021 | | | Symbol; Acc: 8066] | | | |
| | control | | | 3024 | | | | | | |
| ADXGo | Almac | Sense | 1 | ENS | SO | 122809 | suppressor of cytokine | Chr 14 | Forward | N/A |
| od72_at | pixe- | (Fully | | G000 | CS | | signaling 4 [Source: HGNC | | Strand | |
| | llation | Exonic) | | 0018 | 4 | | Symbol; Acc: 19392] | | | |
| | control | | | 0008 | | | | | | |
| ADXGo | Almac | Sense | 1 | ENS | CR | 78987 | cysteine-rich with EGF-like | Chr 3 | Forward | N/A |
| od72_at | pixe- | (Fully | | G000 | EL | | domains 1 [Source: HGNC | | Strand | |
| | llation | Exonic) | | 0016 | D1 | | Symbol; Acc: 14630] | | | |
| | control | | | 3703 | | | | | | |
| OCAD | Expre- | Sense | 11 | ENS | PM | 5376 | peripheral myelin protein 22 | Chr 17 | Reverse | 136 |
| A.9170_ | sion | (includes | | G000 | P22 | | [Source: HGNC | | Strand | |
| s_at | probe | Intronic) | | 0010 | | | Symbol; Acc: 9118] | | | |
| | set | | | 9099 | | | | | | |
| OC3P. | Expre- | Sense | 11 | ENS | PM | 5376 | peripheral myelin protein 22 | Chr 17 | Reverse | 137 |
| 10622. | sion | (Fully | | G000 | P22 | | [Source: HGNC | | Strand | |
| C1_s_at | probe | Exonic) | | 0010 | | | Symbol; Acc: 9118] | | | |
| | set | | | 9099 | | | | | | |
| OC3SN | Expre- | Sense | 11 | ENS | PM | 5376 | peripheral myelin protein 22 | Chr 17 | Reverse | 138 |
| Gnh.89 | sion | (includes | | G000 | P22 | | [Source: HGNC | | Strand | |
| 44_s_at | probe | Intronic) | | 0010 | | | Symbol; Acc: 9118] | | | |
| | set | | | 9099 | | | | | | |
| OC3SN | Expre- | Sense | 11 | ENS | PO | 5425 | polymerase (DNA directed), | Chr 7 | Reverse | 139 |
| G.4571- | sion | (Fully | | G000 | LD | | delta 2, accessory subunit | | Strand | |
| 22a_x_ | probe | Exonic) | | 0010 | 2 | | [Source: HGNC | | | |
| at | set | | | 6628 | | | Symbol; Acc: 9176] | | | |
| OCEM. | Expre- | Sense | 11 | ENS | PO | 5425 | polymerase (DNA directed), | Chr 7 | Reverse | 140 |
| 1126_ | sion | (Fully | | G000 | LD | | delta 2, accessory subunit | | Strand | |
| s_at | probe | Exonic) | | 0010 | 2 | | [Source: HGNC | | | |
| | set | | | 6628 | | | Symbol; Acc: 9176] | | | |
| ADXGo | Almac | Sense | 1 | ENS | FO | 2308 | forkhead box 01 | Chr 13 | Reverse | N/A |
| od4_at | pixe- | (Fully | | G000 | XO | | [Source: HGNC | | Strand | |
| | llation | Exonic) | | 0015 | 1 | | Symbol; Acc: 3819] | | | |
| | control | | | 0907 | | | | | | |
| ADXGo | Almac | Sense | 1 | ENS | OC | 4952 | oculocerebrorenal syndrome of | Chr X | Forward | N/A |
| od4_at | pixe- | (Fully | | G000 | RL | | Lowe [Source: HGNC | | Strand | |
| | llation | Exonic) | | 0012 | | | Symbol; Acc: 8108] | | | |
| | control | | | 2126 | | | | | | |
| ADXGo | Almac | Sense | 1 | ENS | PO | 5425 | polymerase (DNA directed), | Chr 7 | Reverse | N/A |
| od4_at | pixe- | (Fully | | G000 | LD | | delta 2, accessory subunit | | Strand | |
| | llation | Exonic) | | 0010 | 2 | | [Source: HGNC | | | |
| | control | | | 6628 | | | Symbol; Acc: 9176] | | | |
| ADXGo | Almac | Sense | 1 | ENS | PIK | 8503 | phosphoinositide-3-kinase, | Chr 1 | Reverse | N/A |
| od4_at | pixe- | (Fully | | G000 | 3R | | regulatory subunit 3 (gamma) | | Strand | |
| | llation | Exonic) | | 0011 | 3 | | [Source: HGNC | | | |
| | control | | | 7461 | | | Symbol; Acc: 8981] | | | |
| ADXGo | Almac | Sense | 1 | ENS | S10 | 6281 | S100 calcium binding protein | Chr 1 | Reverse | N/A |
| od4_at | pixe- | (Fully | | G000 | 0A1 | | A10 [Source: HGNC | | Strand | |
| | llation | Exonic) | | 0019 | 0 | | Symbol; Acc: 10487] | | | |
| | control | | | 7747 | | | | | | |
| ADXGo | Almac | Sense | 1 | ENS | TA | 6892 | TAP binding protein (tapasin) | Chr 6 | Reverse | N/A |
| od4_at | pixe- | (Fully | | G000 | PB | | [Source: HGNC | | Strand | |
| | llation | Exonic) | | 0023 | P | | Symbol; Acc: 11566] | | | |
| | control | | | 1925 | | | | | | |
| ADXGo | Almac | Sense | 1 | ENS | RU | 80230 | RUN and FYVE domain | Chr 5 | Forward | N/A |
| od4_at | pixe- | (Fully | | G000 | FY | | containing 1 [Source: HGNC | | Strand | |
| | llation | Exonic) | | 0017 | 1 | | Symbol; Acc: 19760] | | | |
| | control | | | 6783 | | | | | | |
| ADXGo | Almac | Sense | 1 | ENS | SL | 6574 | solute carrier family 20 | Chr 2 | Forward | N/A |
| od4_at | pixe- | (Fully | | G000 | C2 | | (phosphate transporter), | | Strand | |
| | llation | Exonic) | | 0014 | 0A1 | | member 1 [Source: HGNC | | | |
| | control | | | 4136 | | | Symbol; Acc: 10946] | | | |
| ADXGo | Almac | Sense | 1 | ENS | US | 7398 | ubiquitin specific peptidase 1 | Chr 1 | Forward | N/A |
| od4_at | pixe- | (Fully | | G000 | P1 | | [Source: HGNC | | Strand | |
| | llation | Exonic) | | 0016 | | | Symbol; Acc: 12607] | | | |
| | control | | | 2607 | | | | | | |
| ADXGo | Almac | Sense | 1 | ENS | HIS | 3006 | histone cluster 1, H1c | Chr 6 | Reverse | N/A |
| od4_at | pixe- | (Fully | | G000 | T1 | | [Source: HGNC | | Strand | |
| | llation | Exonic) | | 0018 | H1 | | Symbol; Acc: 4716] | | | |
| | control | | | 7837 | C | | | | | |
| OC3SN | Expre- | Sense | 11 | ENS | PO | 5425 | polymerase (DNA directed), | Chr 7 | Reverse | 141 |
| Gn.890- | sion | (Fully | | G000 | LD | | delta 2, accessory subunit | | Strand | |
| 5a_s_ | probe | Exonic) | | 0010 | 2 | | [Source: HGNC | | | |
| at | set | | | 6628 | | | Symbol; Acc: 9176] | | | |
| OCAD | Expre- | Sense | 11 | ENS | PO | 10631 | periostin, osteoblast specific | Chr 13 | Reverse | 142 |
| A.7987_ | sion | (Fully | | G000 | ST | | factor [Source: HGNC | | Strand | |
| s_at | probe | Exonic) | | 0013 | N | | Symbol; Acc: 16953] | | | |
| | set | | | 3110 | | | | | | |
| OC3SN | Expre- | Sense | 11 | ENS | PO | 10631 | periostin, osteoblast specific | Chr 13 | Reverse | 143 |
| Gnh.57 | sion | (Fully | | G000 | ST | | factor [Source: HGNC | | Strand | |
| 24_at | probe | Exonic) | | 0013 | N | | Symbol; Acc: 16953] | | | |
| | set | | | 3110 | | | | | | |
| OCHP. | Expre- | Sense | 11 | ENS | PO | 10631 | periostin, osteoblast specific | Chr 13 | Reverse | 144 |
| 402_s_ | sion | (Fully | | G000 | ST | | factor [Source: HGNC | | Strand | |
| at | probe | Exonic) | | 0013 | N | | Symbol; Acc: 16953] | | | |
| | set | | | 3110 | | | | | | |
| OC3P. | Expre- | Sense | 11 | ENS | PO | 10631 | periostin, osteoblast specific | Chr 13 | Reverse | 145 |
| 1013.C | sion | (Fully | | G000 | ST | | factor [Source: HGNC | | Strand | |
| 1_x_at | probe | Exonic) | | 0013 | N | | Symbol; Acc: 16953] | | | |
| | set | | | 3110 | | | | | | |
| OC3P. | Expre- | Sense | 6 | ENS | PO | 10631 | periostin, osteoblast specific | Chr 13 | Reverse | 146 |
| 1013.C | sion | (Fully | | G000 | ST | | factor [Source: HGNC | | Strand | |
| 2_x_at | probe | Exonic) | | 0013 | N | | Symbol; Acc: 16953] | | | |
| | set | | | 3110 | | | | | | |
| OCAD | Expre- | Sense | 11 | ENS | PO | 10631 | periostin, osteoblast specific | Chr 13 | Reverse | 147 |
| NP.115 | sion | (includes | | G000 | ST | | factor [Source: HGNC | | Strand | |
| 85_s_ | probe | Intronic) | | 0013 | N | | Symbol; Acc: 16953] | | | |
| at | set | | | 3110 | | | | | | |
| OC3P. | Expre- | Sense | 11 | ENS | PO | 10631 | periostin, osteoblast specific | Chr 13 | Reverse | 148 |
| 1013.C | sion | (Fully | | G000 | ST | | factor [Source: HGNC | | Strand | |
| 1_s_at | probe | Exonic) | | 0013 | N | | Symbol; Acc: 16953] | | | |
| | set | | | 3110 | | | | | | |
| OC3SN | Expre- | Sense | 11 | ENS | PO | 10631 | periostin, osteoblast specific | Chr 13 | Reverse | 149 |
| Gnh.57 | sion | (Fully | | G000 | ST | | factor [Source: HGNC | | Strand | |
| 24_x_ | probe | Exonic) | | 0013 | N | | Symbol; Acc: 16953] | | | |
| at | set | | | 3110 | | | | | | |
| OC3P. | Expre- | Sense | 11 | ENS | RA | 11031 | RAB31, member RAS | Chr 18 | Forward | 150 |
| 8262.0 | sion | (Fully | | G000 | B31 | | oncogene family | | Strand | |
| 1_s_at | probe | Exonic) | | 0016 | | | [Source: HGNC | | | |
| | set | | | 8461 | | | Symbol; Acc: 9771] | | | |
| OC3SN | Expre- | Sense | 11 | ENS | RA | 11031 | RAB31, member RAS | Chr 18 | Forward | 151 |
| Gnh.17 | sion | (includes | | G000 | B31 | | oncogene family | | Strand | |
| 870_s_ | probe | Intronic) | | 0016 | | | [Source: HGNC | | | |
| at | set | | | 8461 | | | Symbol; Acc: 9771] | | | |
| OC3P. | Expre- | Sense | 11 | ENS | RA | 11031 | RAB31, member RAS | Chr 18 | Forward | 152 |
| 11285. | sion | (Fully | | G000 | B31 | | oncogene family | | Strand | |
| C1_s_ | probe | Exonic) | | 0016 | | | [Source: HGNC | | | |
| at | set | | | 8461 | | | Symbol; Acc: 9771] | | | |
| OCHP. | Expre- | Sense | 11 | ENS | RA | 11031 | RAB31, member RAS | Chr 18 | Forward | 153 |
| 1160_ | sion | (Fully | | G000 | B31 | | oncogene family | | Strand | |
| s_at | probe | Exonic) | | 0016 | | | [Source: HGNC | | | |
| | set | | | 8461 | | | Symbol; Acc: 9771] | | | |
| OCMX. | Expre- | Sense | 10 | ENS | RA | 11031 | RAB31, member RAS | Chr 18 | Forward | 154 |
| 11222. | sion | (includes | | G000 | B31 | | oncogene family | | Strand | |
| C1_at | probe | Intronic) | | 0016 | | | [Source: HGNC | | | |
| | set | | | 8461 | | | Symbol; Acc: 9771] | | | |
| OC3SN | Expre- | Sense | 11 | ENS | RU | 860 | runt-related transcription factor | Chr 6 | Forward | 155 |
| Gnh.14 | sion | (includes | | G000 | NX | | 2 [Source: HGNC | | Strand | |
| 334_x_ | probe | Intronic) | | 0012 | 2 | | Symbol; Acc: 10472] | | | |
| at | set | | | 4813 | | | | | | |
| OCAD | Expre- | Sense | 11 | ENS | RU | 860 | runt-related transcription factor | Chr 6 | Forward | 156 |
| A.8000_ | sion | (Fully | | G000 | NX2 | | [Source: HGNC | | Strand | |
| s_at | probe | Exonic) | | 0012 | 2 | | Symbol; Acc: 10472] | | | |
| | set | | | 4813 | | | | | | |
| OCAD | Expre- | Sense | 11 | ENS | RU | 860 | runt-related transcription factor | Chr 6 | Forward | 157 |
| NP.631 | sion | (Fully | | G000 | NX | | 2 [Source: HGNC | | Strand | |
| 5_s_at | probe | Exonic) | | 0012 | 2 | | Symbol; Acc: 10472] | | | |
| | set | | | 4813 | | | | | | |
| OCMX. | Expre- | Sense | 9 | ENS | RU | 860 | runt-related transcription factor | Chr 6 | Forward | 158 |
| 1543.C | sion | (Fully | | G000 | NX | | 2 [Source: HGNC | | Strand | |
| 1_s_at | probe | Exonic) | | 0012 | 2 | | Symbol; Acc: 10472] | | | |
| | set | | | 4813 | | | | | | |
| OCHP. | Expre- | Sense | 11 | ENS | RU | 860 | runt-related transcription factor | Chr 6 | Forward | 159 |
| 1038_ | sion | (Fully | | G000 | NX | | 2 [Source: HGNC | | Strand | |
| s_at | probe | Exonic) | | 0012 | 2 | | Symbol; Acc: 10472] | | | |
| | set | | | 4813 | | | | | | |
| OCHP. | Expre- | Sense | 11 | ENS | SE | 5176 | serpin peptidase inhibitor, clade | Chr 17 | Forward | 160 |
| 781_s_ | sion | (Fully | | G000 | RPI | | F (alpha-2 antiplasmin, pigment | | Strand | |
| at | probe | Exonic) | | 0013 | NF | | epithelium derived factor), | | | |
| | set | | | 2386 | 1 | | member 1 [Source: HGNC | | | |
| | | | | | | | Symbol; Acc: 8824] | | | |
| ADXStr | Almac | Sense | 1 | ENS | AR | 410 | arylsulfatase A [Source: HGNC | Chr 22 | Reverse | N/A |
| ong15_ | pixe- | (Fully | | G000 | SA | | Symbol; Acc: 713] | | Strand | |
| at | llation | Exonic) | | 0010 | | | | | | |
| | control | | | 0299 | | | | | | |
| ADXStr | Almac | Sense | 1 | ENS | MT | 9219 | metastasis associated 1 family, | Chr 11 | Reverse | N/A |
| ong15_ | pixe- | (Fully | | G000 | A2 | | member 2 [Source: HGNC | | Strand | |
| at | llation | Exonic) | | 0014 | | | Symbol; Acc: 7411] | | | |
| | control | | | 9480 | | | | | | |
| ADXStr | Almac | Sense | 1 | ENS | ND | N/A | NADH dehydrogenase | Chr 1 | Forward | N/A |
| ong15_ | pixe- | (Fully | | G000 | UF | | (ubiquinone) Fe-S protein 5, | | Strand | |
| at | llation | Exonic) | | 0023 | S5 | | 15 kDa (NADH-coenzyme Q | | | |
| | control | | | 3664 | P3 | | reductase) pseudogene 3 | | | |
| | | | | | | | [Source: HGNC | | | |
| | | | | | | | Symbol; Acc: 44041] | | | |
| ADXStr | Almac | Sense | 1 | ENS | ND | 4725 | NADH dehydrogenase | Chr 1 | Forward | N/A |
| ong15_ | pixe- | (Fully | | G000 | UF | | (ubiquinone) Fe-S protein 5, | | Strand | |
| at | llation | Exonic) | | 0016 | S5 | | 15 kDa (NADH-coenzyme Q | | | |
| | control | | | 8653 | | | reductase) [Source: HGNC | | | |
| | | | | | | | Symbol; Acc: 7712] | | | |
| ADXStr | Almac | Sense | 1 | ENS | SN | 9410 | small nuclear ribonucleoprotein | Chr 1 | Reverse | N/A |
| ong15_ | pixe- | (Fully | | G000 | RN | | 40 kDa (U5) [Source: HGNC | | Strand | |
| at | tion | Exonic) | | 0006 | P40 | | Symbol; Acc: 30857] | | | |
| | control | | | 0688 | | | | | | |
| ADXStr | Almac | Sense | 1 | ENS | DN | 1785 | dynamin 2 [Source: HGNC | Chr 19 | Forward | N/A |
| ong15_ | pixe- | (Fully | | G000 | M2 | | Symbol; Acc: 2974] | | Strand | |
| at | llation | Exonic) | | 0007 | | | | | | |
| | control | | | 9805 | | | | | | |
| ADXStr | Almac | Sense | 1 | ENS | BT | 694 | B-cell translocation gene 1, | Chr 12 | Reverse | N/A |
| ong15_ | pixe- | (Fully | | G000 | G1 | | anti-proliferative [Source: | | Strand | |
| at | llation | Exonic) | | 0013 | | | HGNC Symbol; Acc: 1130] | | | |
| | control | | | 3639 | | | | | | |
| ADXStr | Almac | Sense | 1 | ENS | ZN | 162993/// | zinc finger protein 846 | Chr 19 | Reverse | N/A |
| ong15_ | pixe- | (Fully | | G000 | F84 | 100505 | [Source: HGNC | | Strand | |
| at | llation | Exonic) | | 0019 | 6 | 555 | Symbol; Acc: 27260] | | | |
| | control | | | 6605 | | | | | | |
| ADXStr | Almac | Sense | 1 | ENS | SE | 5176 | serpin peptidase inhibitor, clade | Chr 17 | Forward | N/A |
| ong15_ | pixe- | (Fully | | G000 | RPI | | F (alpha-2 antiplasmin, pigment | | Strand | |
| at | llation | Exonic) | | 0013 | NF | | epithelium derived factor), | | | |
| | control | | | 2386 | 1 | | member 1 [Source: HGNC | | | |
| | | | | | | | Symbol; Acc: 8824] | | | |
| ADXStr | Almac | Sense | 1 | ENS | PIG | 5284 | polymeric immunoglobulin | Chr 1 | Reverse | N/A |
| ong15_ | pixe- | (Fully | | G000 | R | | receptor [Source: HGNC | | Strand | |
| at | llation | Exonic) | | 0016 | | | Symbol; Acc: 8968] | | | |
| | control | | | 2896 | | | | | | |
| ADXStr | Almac | Sense | 1 | ENS | ND | N/A | NADH dehydrogenase | Chr 4 | Reverse | N/A |
| ong15_ | pixe- | (Fully | | G000 | UF | | (ubiquinone) Fe—S protein 5, | | Strand | |
| at | llation | Exonic) | | 0023 | S5 | | 15 kDa (NADH-coenzyme Q | | | |
| | control | | | 0671 | P5 | | reductase) pseudogene 5 | | | |
| | | | | | | | [Source: HGNC | | | |
| | | | | | | | Symbol; Acc: 44043] | | | |
| ADXStr | Almac | Sense | 1 | ENS | EZ | 7430 | ezrin [Source: HGNC | Chr 6 | Reverse | N/A |
| ong15_ | pixe- | (Fully | | G000 | R | | Symbol; Acc: 12691] | | Strand | |
| at | llation | Exonic) | | 0009 | | | | | | |
| | control | | | 2820 | | | | | | |
| OCEM. | Expre- | Sense | 11 | ENS | SE | 5176 | serpin peptidase inhibitor, clade | Chr 17 | Forward | 161 |
| 1960_ | sion | (Fully | | G000 | RPI | | F (alpha-2 antiplasmin, pigment | | Strand | |
| at | probe | Exonic) | | 0013 | NF | | epithelium derived factor), | | | |
| | set | | | 2386 | 1 | | member 1 [Source: HGNC | | | |
| | | | | | | | Symbol; Acc: 8824] | | | |
| ADXStr | Almac | Sense | 1 | ENS | N/A | N/A | KNOWN pseudogene | Chr 11 | Reverse | N/A |
| ong8_ | pixe- | (Fully | | G000 | | | (Clone_based_vega_gene) | | Strand | |
| at | llation | Exonic) | | 0025 | | | | | | |
| | control | | | 4612 | | | | | | |
| ADXStr | Almac | Sense | 1 | ENS | DN | 10049 | DnaJ (Hsp40) homolog, | Chr 7 | Forward | N/A |
| ong8_ | pixe- | (Fully | | G000 | AJ | | subfamily B, member 6 | | Strand | |
| at | llation | Exonic) | | 0010 | B6 | | [Source: HGNC | | | |
| | control | | | 5993 | | | Symbol; Acc: 14888] | | | |
| ADXStr | Almac | Sense | 1 | ENS | ZF | 7543 | zinc finger protein, X-linked | Chr X | Forward | N/A |
| ong8_ | pixe- | (Fully | | G000 | X | | [Source: HGNC | | Strand | |
| at | llation | Exonic) | | G000 | | | Symbol; Acc: 12869] | | | |
| | control | | | 5889 | | | | | | |
| ADXStr | Almac | Sense | 1 | ENS | DN | 3300 | DnaJ (Hsp40) homolog, | Chr 2 | Forward | N/A |
| ong8_ | pixe- | (Fully | | G000 | AJ | | subfamily B, member 2 | | Strand | |
| at | llation | Exonic) | | 0013 | B2 | | [Source: HGNC | | | |
| | control | | | 5924 | | | Symbol; Acc: 5228] | | | |
| ADXStr | Almac | Sense | 1 | ENS | GA | 2617 | glycyl-tRNA synthetase | Chr 7 | Forward | N/A |
| ong8_ | pixe- | (Fully | | G000 | RS | | [Source: HGNC | | Strand | |
| at | llation | Exonic) | | 0010 | | | Symbol; Acc: 4162] | | | |
| | control | | | 6105 | | | | | | |
| ADXStr | Almac | Sense | 1 | ENS | RF | 64326 | ring finger and WD repeat | Chr 1 | Reverse | N/A |
| ong8_ | pixe- | (Fully | | G000 | WD | | domain 2, E3 ubiquitin protein | | Strand | |
| at | llation | Exonic) | | 0014 | 2 | | ligase [Source: HGNC | | | |
| | control | | | 3207 | | | Symbol; Acc: 17440] | | | |
| ADXStr | Almac | Sense | 1 | ENS | MX | 4599 | myxovirus (influenza virus) | Chr 21 | Forward | N/A |
| ong8_ | pixe- | (Fully | | G000 | 1 | | resistance 1, interferon- | | Strand | |
| at | llation | Exonic) | | 0015 | | | inducible protein p78 (mouse) | | | |
| | control | | | 7601 | | | [Source: HGNC | | | |
| | | | | | | | Symbol; Acc: 7532] | | | |
| ADXStr | Almac | Sense | 1 | ENS | SE | 5176 | serpin peptidase inhibitor, clade | | | |
| ong8_ | pixe- | (Fully | | G000 | RPI | | F (alpha-2 antiplasmin, pigment | Chr 17 | Forward | N/A |
| at | llation | Exonic) | | 0013 | NF | | epithelium derived factor), | | Strand | |
| | control | | | 2386 | 1 | | member 1 [Source: HGNC | | | |
| | | | | | | | Symbol; Acc: 8824] | | | |
| ADXStr | Almac | Sense | 1 | ENS | N/A | N/A | NOVEL protein_coding | Chr 19 | Reverse | N/A |
| ong8_ | pixe- | (Fully | | G000 | | | (Clone_based_vega_gene) | | Strand | |
| at | llation | Exonic) | | 0026 | | | | | | |
| | control | | | 9242 | | | | | | |
| ADXStr | Almac | Sense | 1 | ENS | PIG | 51604 | phosphatidylinositol glycan | Chr 20 | Forward | N/A |
| ong8_ | pixe- | (Fully | | G000 | T | | anchor biosynthesis, class T | | Strand | |
| at | llation | Exonic) | | 0012 | | | [Source: HGNC | | | |
| | control | | | 4155 | | | Symbol; Acc: 14938] | | | |
| ADXStr | Almac | Sense | 1 | ENS | TP | 7177 | tryptase alpha/beta 1 | Chr 16 | Forward | N/A |
| ong8_ | pixe- | (Fully | | G000 | SA | | [Source: HGNC | | Strand | |
| at | llation | Exonic) | | 0017 | B1 | | Symbol; Acc: 12019] | | | |
| | control | | | 2236 | | | | | | |
| ADXStr | Almac | Sense | 1 | ENS | ND | 4726 | NADH dehydrogenase | Chr 5 | Forward | N/A |
| ong8_ | pixe- | (Fully | | G000 | UF | | (ubiquinone) Fe-S protein 6, | | Strand | |
| at | llation | Exonic) | | 0014 | S6 | | 13 kDa (NADH-coenzyme Q | | | |
| | control | | | 5494 | | | reductase) [Source: HGNC | | | |
| | | | | | | | Symbol; Acc: 7713] | | | |
| ADXStr | Almac | Sense | 1 | ENS | TP | 64499 | tryptase beta 2 | Chr 16 | Reverse | N/A |
| ong8_ | pixe- | (Fully | | G000 | SB | | (gene/pseudogene) | | Strand | |
| at | llation | Exonic) | | 0019 | 2 | | [Source: HGNC | | | |
| | control | | | 7253 | | | Symbol; Acc: 14120] | | | |
| ADXStr | Almac | Sense | 1 | ENS | MA | 4125 | mannosidase, alpha, class 2B, | Chr 19 | Reverse | N/A |
| ong8_ | pixe- | (Fully | | G000 | N2 | | member 1 [Source: HGNC | | Strand | |
| at | llation | Exonic) | | 0010 | B1 | | Symbol; Acc: 6826] | | | |
| | control | | | 4774 | | | | | | |
| OC3SN | Expre- | Sense | 11 | ENS | SF | 6423 | secreted frizzled-related protein | Chr 4 | Reverse | 162 |
| Gn.251- | sion | (Fully | | G000 | RP | | 2 [Source: HGNC | | Strand | |
| 21a_s_ | probe | Exonic) | | 0014 | 2 | | Symbol; Acc: 10777] | | | |
| at | set | | | 5423 | | | | | | |
| OC3P. | Expre- | Sense | 11 | ENS | SF | 6423 | secreted frizzled-related protein | Chr 4 | Reverse | 163 |
| 13621. | sion | (Fully | | G000 | RP | | 2 [Source: HGNC | | Strand | |
| C1_s_ | probe | Exonic) | | 0014 | 2 | | Symbol; Acc: 10777] | | | |
| at | set | | | 5423 | | | | | | |
| OCHP. | Expre- | Sense | 11 | ENS | TH | 7058 | thrombospondin 2 | Chr 6 | Reverse | 164 |
| 677_s_ | sion | (Fully | | G000 | BS | | [Source: HGNC | | Strand | |
| at | probe | Exonic) | | 0018 | 2 | | Symbol; Acc: 11786] | | | |
| | set | | | 6340 | | | | | | |
| OC3P. | Expre- | Sense | 11 | ENS | TH | 7058 | thrombospondin 2 | Chr 6 | Reverse | 165 |
| 4296.C | sion | (Fully | | G000 | BS | | [Source: HGNC | | Strand | |
| 2_s_at | probe | Exonic) | | 0018 | 2 | | Symbol; Acc: 11786] | | | |
| | set | | | 6340 | | | | | | |
| OC3SN | Expre- | Sense | 11 | ENS | TH | 7058 | thrombospondin 2 | Chr 6 | Reverse | 166 |
| Gnh.14 | sion | (includes | | G000 | BS | | [Source: HGNC | | Strand | |
| 530_s_ | probe | Intronic) | | 0018 | 2 | | Symbol; Acc: 11786] | | | |
| at | set | | | 6340 | | | | | | |
| OC3SN | Expre- | Sense | 11 | ENS | TH | 7058 | thrombospondin 2 | Chr 6 | Reverse | 167 |
| Gnh.14 | sion | (includes | | G000 | BS | | [Source: HGNC | | Strand | |
| 530_at | probe | Intronic) | | 0018 | 2 | | Symbol; Acc: 11786] | | | |
| | set | | | 6340 | | | | | | |
| OC3P. | Expre- | Sense | 11 | ENS | TH | 7058 | thrombospondin 2 | Chr 6 | Reverse | 168 |
| 4296.C | sion | (Fully | | G000 | BS | | [Source: HGNC | | Strand | |
| 1- | probe | Exonic) | | 0018 | 2 | | Symbol; Acc: 11786] | | | |
| 409a_s | set | | | 6340 | | | | | | |
| at | | | | | | | | | | |
| OC3SN | Expre- | Sense | 11 | ENS | TH | 7058 | thrombospondin 2 | Chr 6 | Reverse | 169 |
| Gn.187 | sion | (Fully | | G000 | BS | | [Source: HGNC | | Strand | |
| 3- | probe | Exonic) | | 0018 | 2 | | Symbol; Acc: 11786] | | | |
| 1656a_ | set | | | 6340 | | | | | | |
| s_at | | | | | | | | | | |
| OCAD | Expre- | Sense | 11 | ENS | TIM | 7078 | TIMP metallopeptidase | Chr 22 | Forward | 170 |
| NP.130 | sion | (includes | | G000 | P3 | | inhibitor 3 [Source: HGNC | | Strand | |
| 17_s_ | probe | Intronic) | | 0010 | | | Symbol; Acc: 11822] | | | |
| at | set | | | 0234 | | | | | | |
| OCAD | Expre- | Sense | 11 | ENS | TIM | 7078 | TIMP metallopeptidase | Chr 22 | Forward | 171 |
| A.9324_ | sion | (Fully | | G000 | P3 | | inhibitor 3 [Source: HGNC | | Strand | |
| s_at | probe | Exonic) | | 0010 | | | Symbol; Acc: 11822] | | | |
| | set | | | 0234 | | | | | | |
| OCHP. | Expre- | Sense | 11 | ENS | TIM | 7078 | TIMP metallopeptidase | Chr 22 | Forward | 172 |
| 1200_ | sion | (Fully | | G000 | P3 | | inhibitor 3 [Source: HGNC | | Strand | |
| s_at | probe | Exonic) | | 0010 | | | Symbol; Acc: 11822] | | | |
| | set | | | 0234 | | | | | | |
| ADXGo | Almac | Sense | 1 | ENS | RA | 5899 | v-ral simian leukemia viral | Chr 2 | Forward | N/A |
| od73_ | pixe- | (Fully | | G000 | LB | | oncogene homolog B | | Strand | |
| at | llation | Exonic) | | 0014 | | | [Source: HGNC | | | |
| | control | | | 4118 | | | Symbol; Acc: 9840] | | | |
| ADXGo | Almac | Sense | 1 | ENS | PR | 5627 | protein S (alpha) | Chr 3 | Reverse | N/A |
| od73_ | pixe- | (Fully | | G000 | OS | | [Source: HGNC | | Strand | |
| at | llation | Exonic) | | 0018 | 1 | | Symbol; Acc: 9456] | | | |
| | control | | | 4500 | | | | | | |
| ADXGo | Almac | Sense | 1 | ENS | AD | 109 | adenylate cyclase 3 | Chr 2 | Reverse | N/A |
| od73_ | pixe- | (Fully | | G000 | CY | | [Source: HGNC | | Strand | |
| at | llation | Exonic) | | 0013 | 3 | | Symbol; Acc: 234] | | | |
| | control | | | 8031 | | | | | | |
| ADXGo | Almac | Sense | 1 | ENS | TIM | 7078 | TIMP metallopeptidase inhibitor | Chr 22 | Forward | N/A |
| od73_ | pixe- | (Fully | | G000 | P3 | | 3 [Source: HGNC | | Strand | |
| at | llation | Exonic) | | 0010 | | | Symbol; Acc: 11822] | | | |
| | control | | | 0234 | | | | | | |
| ADXGo | Almac | Sense | 1 | ENS | DK | 1736/// | dyskeratosis congenita 1, | Chr X | Forward | N/A |
| od73_ | pixe- | (Fully | | G000 | C1 | 677835/// | dyskerin [Source: HGNC | | Strand | |
| at | llation | Exonic) | | 0013 | | 100847 | Symbol; Acc: 2890] | | | |
| | control | | | 0826 | | 052 | | | | |
| ADXGo | Almac | Sense | 1 | ENS | ED | 8721 | endothelial differentiation- | Chr 9 | Reverse | N/A |
| od73_ | pixe- | (Fully | | G000 | F1 | | related factor 1 [Source: HGNC | | Strand | |
| at | llation | Exonic) | | 0010 | | | Symbol; Acc: 3164] | | | |
| | control | | | 7223 | | | | | | |
| ADXGo | Almac | Sense | 1 | ENS | LA | 3913 | laminin, beta 2 (laminin S) | Chr 3 | Reverse | N/A |
| od73_ | pixe- | (Fully | | G000 | MB | | [Source: HGNC | | Strand | |
| at | llation | Exonic) | | 0017 | 2 | | Symbol; Acc: 6487] | | | |
| | control | | | 2037 | | | | | | |
| ADXGo | Almac | Sense | 1 | ENS | SG | 6449 | small glutamine-rich | Chr 19 | Reverse | N/A |
| od73_ | pixe- | (Fully | | G000 | TA | | tetratricopeptide repeat (TPR)- | | Strand | |
| at | llation | Exonic) | | 0010 | | | containing, alpha | | | |
| | control | | | 4969 | | | [Source: HGNC | | | |
| | | | | | | | Symbol; Acc: 10819] | | | |
| ADXGo | Almac | Sense | 1 | ENS | YA | 8565 | tyrosyl-tRNA synthetase | Chr 1 | Reverse | N/A |
| od73_ | pixe- | (Fully | | G000 | RS | | [Source: HGNC | | Strand | |
| at | llation | Exonic) | | 0013 | | | Symbol; Acc: 12840] | | | |
| | control | | | 4684 | | | | | | |
| ADXGo | Almac | Sense | 1 | ENS | AIP | 9049 | aryl hydrocarbon receptor | Chr 11 | Forward | N/A |
| od73_ | pixe- | (Fully | | G000 | | | interacting protein | | Strand | |
| at | llation | Exonic) | | 0011 | | | [Source: HGNC | | | |
| | control | | | 0711 | | | Symbol; Acc: 358] | | | |
| ADXGo | Almac | Sense | 1 | ENS | FA | 644815 | family with sequence | Chr 17 | Reverse | N/A |
| od73_ | pixe- | (Fully | | G000 | M8 | | similarity 83, member G | | Strand | |
| at | llation | Exonic) | | 0018 | 30 | | [Source: HGNC Symbol; | | | |
| | control | | | 8522 | | | Acc: 32554] | | | |
| OC3P. | Expre- | Sense | 11 | ENS | TIM | 7078 | TIMP metallopeptidase | Chr 22 | Forward | 173 |
| 10470. | sion | (Fully | | G000 | P3 | | inhibitor 3 [Source: HGNC | | Strand | |
| C1_s_ | probe | Exonic) | | 0010 | | | Symbol; Acc: 11822] | | | |
| at | set | | | 0234 | | | | | | |
| OC3P. | Expre- | Sense | 8 | ENS | TIM | 7078 | TIMP metallopeptidase | Chr 22 | Forward | 174 |
| 15327. | sion | (includes | | G000 | P3 | | inhibitor 3 [Source: HGNC | | Strand | |
| C1_at | probe | Intronic) | | 0010 | | | Symbol; Acc: 11822] | | | |
| | set | | | 0234 | | | | | | |
| OCHP. | Expre- | Sense | 11 | ENS | TIM | 7078 | TIMP metallopeptidase | Chr 22 | Forward | 175 |
| 112_s_ | sion | (Fully | | G000 | P3 | | inhibitor 3 [Source: HGNC | | Strand | |
| at | probe | Exonic) | | 0010 | | | Symbol; Acc: 11822] | | | |
| | set | | | 0234 | | | | | | |
| OC3P. | Expre- | Sense | 11 | ENS | TM | 114801 | transmembrane protein 200A | Chr 6 | Forward | 176 |
| 6478.C | sion | (Fully | | G000 | EM | | [Source: HGNC | | Strand | |
| 1_s_at | probe | Exonic) | | 0016 | 200 | | Symbol; Acc: 21075] | | | |
| | set | | | 4484 | A | | | | | |
| OC3P. | Expre- | Sense | 11 | ENS | TM | 114801 | transmembrane protein 200A | Chr 6 | Forward | 177 |
| 6478.C | sion | (Fully | | G000 | EM | | [Source: HGNC | | Strand | |
| 1- | probe | Exonic) | | 0016 | 200 | | Symbol; Acc: 21075] | | | |
| 363a_s | set | | | 4484 | A | | | | | |
| at | | | | | | | | | | |
| OCRS | Expre- | Sense | 10 | ENS | TN | 7130 | tumor necrosis factor, alpha- | Chr 2 | Forward | 178 |
| 2.10857 | sion | (Fully | | G000 | FAI | | induced protein 6 | | Strand | |
| _x_at | probe | Exonic) | | 0012 | P6 | | [Source: HGNC | | | |
| | set | | | 3610 | | | Symbol; Acc: 11898] | | | |
| OC3P. | Expre- | Sense | 11 | ENS | VC | 1462 | versican [Source: HGNC | Chr 5 | Forward | 179 |
| 15028. | sion | (Fully | | G000 | AN | | Symbol; Acc: 2464] | | Strand | |
| C1_s_ | probe | Exonic) | | 0003 | | | | | | |
| at | set | | | 8427 | | | | | | |
| OCAD | Expre- | Sense | 8 | ENS | VC | 1462 | versican [Source: HGNC | Chr 5 | Forward | 180 |
| NP.965 | sion | (Fully | | G000 | AN | | Symbol; Acc: 2464] | | Strand | |
| 7_s_at | probe | Exonic) | | 0003 | | | | | | |
| | set | | | 8427 | | | | | | |
| OCMX. | Expre- | Sense | 11 | ENS | VC | 1462 | versican [Source: HGNC | Chr 5 | Forward | 181 |
| 15173. | sion | (Fully | | G000 | AN | | Symbol; Acc: 2464] | | Strand | |
| C1_s_ | probe | Exonic) | | 0003 | | | | | | |
| at | set | | | 8427 | | | | | | |
| OCAD | Expre- | Sense | 11 | ENS | VC | 1462 | versican [Source: HGNC | Chr 5 | Forward | 182 |
| NP.619 | sion | (Fully | | G000 | AN | | Symbol; Acc: 2464] | | Strand | |
| 7_s_at | probe | Exonic) | | 0003 | | | | | | |
| | set | | | 8427 | | | | | | |
| OCRS | Expre- | Sense | 11 | ENS | VC | 1462 | versican [Source: HGNC | Chr 5 | Forward | 183 |
| 2.1143_ | sion | (Fully | | G000 | AN | | Symbol; Acc: 2464] | | Strand | |
| s_at | probe | Exonic) | | 0003 | | | | | | |
| | set | | | 8427 | | | | | | |
| OC3SN | Expre- | Sense | 7 | ENS | VC | 1462 | versican [Source: HGNC | Chr 5 | Forward | 184 |
| Gnh.16 | sion | (includes | | G000 | AN | | Symbol; Acc: 2464] | | Strand | |
| 280_x_ | probe | Intronic) | | 0003 | | | | | | |
| at | set | | | 8427 | | | | | | |
| OC3P. | Expre- | Sense | 11 | ENS | VC | 1462 | versican [Source: HGNC | Chr 5 | Forward | 185 |
| 1200.C | sion | (Fully | | G000 | AN | | Symbol; Acc: 2464] | | Strand | |
| 1_s_at | probe | Exonic) | | 0003 | | | | | | |
| | set | | | 8427 | | | | | | |
| OCAD | Expre- | Sense | 11 | ENS | VG | 389136 | vestigial like 3 (Drosophila) | Chr 3 | Reverse | 186 |
| A.315 | sion | (Fully | | G000 | LL3 | | [Source: HGNC | | Strand | |
| 8_s_at | probe | Exonic) | | 0020 | | | Symbol; Acc: 24327] | | | |
| | set | | | 6538 | | | | | | |
| OCHP. | Expre- | Sense | 11 | ENS | VG | 389136 | vestigial like 3 (Drosophila) | Chr 3 | Reverse | 187 |
| 176_s_ | sion | (Fully | | G000 | LL3 | | [Source: HGNC | | Strand | |
| at | probe | Exonic) | | 0020 | | | Symbol; Acc: 24327] | | | |
| | set | | | 6538 | | | | | | |

TABLE F
15 gene signature probeset information

| | | | No. probes | Ensembl | Gene | Entrez | | | | |
|---|---|---|---|---|---|---|---|---|---|---|
| Probe Set ID | Type | Orientation | aligned | Gene | Symbol | Gene ID | Description | Chromosome | Strand | SEQ ID NO. |
| OC3P.14147.C1_s_at | Expression | Sense | 11 | ENSG00000140937 | CDH11 | 1009 | cadherin 11, type 2, OB-cadherin (osteoblast) | Chr 16 | Reverse | 188 |
| | probeset | (Fully Exonic) | | | | | [Source: HGNC Symbol; Acc: 1750] | | Strand | |
| OC3SNGnh.4032_s_at | Expression | Sense | 11 | ENSG00000140937 | CDH11 | 1009 | cadherin 11, type 2, OB-cadherin (osteoblast) | Chr 16 | Reverse | 189 |
| | probeset | (includes | | | | | [Source: HGNC Symbol; Acc: 1750] | | Strand | |
| | | Intronic) | | | | | | | | |
| OC3SNGnh.5056_x_at | Expression | Sense | 6 | ENSG00000140937 | CDH11 | 1009 | cadherin 11, type 2, OB-cadherin (osteoblast) | Chr 16 | Reverse | 190 |
| | probeset | (includes | | | | | [Source: HGNC Symbol; Acc: 1750] | | Strand | |
| | | Intronic) | | | | | | | | |
| OCADA.6210_s_at | Expression | Sense | 9 | ENSG00000140937 | CDH11 | 1009 | cadherin 11, type 2, OB-cadherin (osteoblast) | Chr 16 | Reverse | 191 |
| | probeset | (includes | | | | | [Source: HGNC Symbol; Acc: 1750] | | Strand | |
| | | Intronic) | | | | | | | | |
| OCADA.8067_x_at | Expression | Sense | 11 | ENSG00000140937 | CDH11 | 1009 | cadherin 11, type 2, OB-cadherin (osteoblast) | Chr 16 | Reverse | 192 |
| | probeset | (including | | | | | [Source: HGNC Symbol; Acc: 1750] | | Strand | |
| | | Intronic) | | | | | | | | |
| OCADNP.10024_s_at | Expression | Sense | 11 | ENSG00000140937 | CDH11 | 1009 | cadherin 11, type 2, OB-cadherin (osteoblast) | Chr 16 | Reverse | 193 |
| | probeset | (Fully Exonic) | | | | | [Source: HGNC Symbol; Acc: 1750] | | Strand | |
| OCHP.148_s_at | Expression | Sense | 11 | ENSG00000140937 | CDH11 | 1009 | cadherin 11, type 2, OB-cadherin (osteoblast) | Chr 16 | Reverse | 194 |
| | probeset | (Fully Exonic) | | | | | [Source: HGNC Symbol; Acc: 1750] | | Strand | |
| OCHPRC.58_s_at | Expression | Sense | 11 | ENSG00000140937 | CDH11 | 1009 | cadherin 11, type 2, OB-cadherin (osteoblast) | Chr 16 | Reverse | 195 |
| | probeset | (Fully Exonic) | | | | | [Source: HGNC Symbol; Acc: 1750] | | Strand | |
| OCMX.1718.C1_s_at | Expression | Sense | 11 | ENSG00000140937 | CDH11 | 1009 | cadherin 11, type 2, OB-cadherin (osteoblast) | Chr 16 | Reverse | 196 |
| | probeset | (Fully Exonic) | | | | | [Source: HGNC Symbol; Acc: 1750] | | Strand | |
| OC3P.11285.C1_s_at | Expression | Sense | 11 | ENSG00000168461 | RAB31 | 11031 | RAB31, member RAS oncogene family | Chr 18 | Forward | 197 |
| | probeset | (Fully Exonic) | | | | | [Source: HGNC Symbol; Acc: 9771] | | Strand | |
| OC3P.8262.C1_s_at | Expression | Sense | 11 | ENSG00000168461 | RAB31 | 11031 | RAB31, member RAS oncogene family | Chr 18 | Forward | 198 |
| | probeset | (Fully Exonic) | | | | | [Source: HGNC Symbol; Acc: 9771] | | Strand | |
| OC3SNGnh.17870_s_at | Expression | Sense | 11 | ENSG00000168461 | RAB31 | 11031 | RAB31, member RAS oncogene family | Chr 18 | Forward | 199 |
| | probeset | (includes | | | | | [Source: HGNC Symbol; Acc: 9771] | | Strand | |
| | | Intronic) | | | | | | | | |
| OCHP.1160_s_at | Expression | Sense | 11 | ENSG00000168461 | RAB31 | 11031 | RAB31, member RAS oncogene family | Chr 18 | Forward | 200 |
| | probeset | (Fully Exonic) | | | | | [Source: HGNC Symbol; Acc: 9771] | | Strand | |
| OCMX.11222.C1_at | Expression | Sense | 10 | ENSG00000168461 | RAB31 | 11031 | RAB31, member RAS oncogene family | Chr 18 | Forward | 201 |
| | probeset | (includes | | | | | [Source: HGNC Symbol; Acc: 9771] | | Strand | |
| | | Intronic) | | | | | | | | |
| OC3P.2342.C1-300a_s_at | Expression | Sense | 11 | ENSG00000130635 | COL5A1 | 1289 | collagen, type V, alpha 1 | Chr 9 | Forward | 202 |
| | probeset | (Fully Exonic) | | | | | [Source: HGNC Symbol; Acc: 2209] | | Strand | |
| OC3P.4984.C1-787a_s_at | Expression | Sense | 11 | ENSG00000130635 | COL5A1 | 1289 | collagen, type V, alpha 1 | Chr 9 | Forward | 203 |
| | probeset | (Fully Exonic) | | | | | [Source: HGNC Symbol; Acc: 2209] | | Strand | |
| OC3P.4984.C1_s_at | Expression | Sense | 11 | ENSG00000130635 | COL5A1 | 1289 | collagen, type V, alpha 1 | Chr 9 | Forward | 204 |
| | probeset | (Fully Exonic) | | | | | [Source: HGNC Symbol; Acc: 2209] | | Strand | |
| OC3SNGnh.10085_x_at | Expression | Sense | 7 | ENSG00000130635 | COL5A1 | 1289 | collagen, type V, alpha 1 | Chr 9 | Forward | 205 |
| | probeset | (includes | | | | | [Source: HGNC Symbol; Acc: 2209] | | Strand | |
| | | Intronic) | | | | | | | | |
| OC3SNGnh.11037_at | Expression | Sense | 10 | ENSG00000130635 | COL5A1 | 1289 | collagen, type V, alpha 1 | Chr 9 | Forward | 206 |
| | probeset | (includes | | | | | [Source: HGNC Symbol; Acc: 2209] | | Strand | |
| | | Intronic) | | | | | | | | |
| OC3SNGnh.11037_x_at | Expression | Sense | 10 | ENSG00000130635 | COL5A1 | 1289 | collagen, type V, alpha 1 | Chr 9 | Forward | 207 |
| | probeset | (includes | | | | | [Source: HGNC Symbol; Acc: 2209] | | Strand | |
| | | Intronic) | | | | | | | | |
| OC3SNGnh.17281_at | Expression | Sense | 9 | ENSG00000130635 | COL5A1 | 1289 | collagen, type V, alpha 1 | Chr 9 | Forward | 208 |
| | probeset | (includes | | | | | [Source: HGNC Symbol; Acc: 2209] | | Strand | |
| | | Intronic) | | | | | | | | |
| OCADA.582_s_at | Expression | Sense | 11 | ENSG00000130635 | COL5A1 | 1289 | collagen, type V, alpha 1 | Chr 9 | Forward | 209 |
| | probeset | (includes | | | | | [Source: HGNC Symbol; Acc: 2209] | | Strand | |
| | | Intronic) | | | | | | | | |
| OCHP.1005_s_at | Expression | Sense | 11 | ENSG00000130635 | COL5A1 | 1289 | collagen, type V, alpha 1 | Chr 9 | Forward | 210 |
| | probeset | (Fully Exonic) | | | | | [Source: HGNC Symbol; Acc: 2209] | | Strand | |
| OCMX.8587.C1_s_at | Expression | Sense | 9 | ENSG00000130635 | COL5A1 | 1289 | collagen, type V, alpha 1 | Chr 9 | Forward | 211 |
| | probeset | (Fully Exonic) | | | | | [Source: HGNC Symbol; Acc: 2209] | | Strand | |
| OC3SNG.1834-947a_s_at | Expression | Sense | 11 | ENSG00000123500 | COL10A1 | 1300 | collagen, type X, alpha 1 | Chr 6 | Reverse | 212 |
| | probeset | (Fully Exonic) | | | | | [Source: HGNC Symbol; Acc: 2185] | | Strand | |
| OCRS.383_s_at | Expression | Sense | 11 | ENSG00000123500 | COL10A1 | 1300 | collagen, type X, alpha 1 | Chr 6 | Reverse | 213 |
| | probeset | (Fully Exonic) | | | | | [Source: HGNC Symbol; Acc: 2185] | | Strand | |
| OC3P.1200.C1_s_at | Expression | Sense | 11 | ENSG00000038427 | VCAN | 1462 | versican | Chr 5 | Forward | 214 |
| | probeset | (Fully Exonic) | | | | | [Source: HGNC Symbol; Acc: 2464] | | Strand | |
| OC3P.15028.C1_s_at | Expression | Sense | 11 | ENSG00000038427 | VCAN | 1462 | versican | Chr 5 | Forward | 215 |
| | probeset | (Fully Exonic) | | | | | [Source: HGNC Symbol; Acc: 2464] | | Strand | |
| OC3SNGnh.16280_x_at | Expression | Sense | 7 | ENSG00000038427 | VCAN | 1462 | versican | Chr 5 | Forward | 216 |
| | probeset | (includes | | | | | [Source: HGNC Symbol; Acc: 2464] | | Strand | |
| | | Intronic) | | | | | | | | |
| OCADNP.6197_s_at | Expression | Sense | 11 | ENSG00000038427 | VCAN | 1462 | versican | Chr 5 | Forward | 217 |
| | probeset | (Fully Exonic) | | | | | [Source: HGNC Symbol; Acc: 2464] | | Strand | |
| OCADNP.9657_s_at | Expression | Sense | 8 | ENSG00000038427 | VCAN | 1462 | versican | Chr 5 | Forward | 218 |
| | probeset | (Fully Exonic) | | | | | [Source: HGNC Symbol; Acc: 2464] | | Strand | |
| OCMX.15173.C1_s_at | Expression | Sense | 11 | ENSG00000038427 | VCAN | 1462 | versican | Chr 5 | Forward | 219 |
| | probeset | (Fully Exonic) | | | | | [Source: HGNC Symbol; Acc: 2464] | | Strand | |
| OCRS2.1143_s_at | Expression | Sense | 11 | ENSG00000038427 | VCAN | 1462 | versican | Chr 5 | Forward | 220 |
| | probeset | (Fully Exonic) | | | | | [Source: HGNC Symbol; Acc: 2464] | | Strand | |
| OC3P.8736.C1_s_at | Expression | Sense | 9 | ENSG00000078098 | FAP | 2191 | fibroblast activation protein, alpha | Chr 2 | Reverse | 221 |
| | probeset | (Fully Exonic) | | | | | [Source: HGNC Symbol; Acc: 3590] | | Strand | |
| OC3SNGn.3016-7a_s_at | Expression | Sense | 8 | ENSG00000078098 | FAP | 2191 | fibroblast activation protein, alpha | Chr 2 | Reverse | 222 |
| | probeset | (Fully Exonic) | | | | | [Source: HGNC Symbol; Acc: 3590] | | Strand | |
| OCADA.9856_x_at | Expression | Sense | 7 | ENSG00000078098 | FAP | 2191 | fibroblast activation protein, alpha | Chr 2 | Reverse | 223 |
| | probeset | (includes | | | | | [Source: HGNC Symbol; Acc: 3590] | | Strand | |
| | | Intronic) | | | | | | | | |
| OC3P.843.CB1-415a_s_at | Expression | Sense | 11 | ENSG00000115414 | FN1 | 2335 | fibronectin 1 | Chr 2 | Reverse | 224 |
| | probeset | (Fully Exonic) | | | | | [Source: HGNC Symbol; Acc: 3778] | | Strand | |
| OC3SNGn.4650-857a_x_at | Expression | Sense | 11 | ENSG00000115414 | FN1 | 2335 | fibronectin 1 | Chr 2 | Reverse | 225 |
| | probeset | (includes | | | | | [Source: HGNC Symbol; Acc: 3778] | | Strand | |
| | | Intronic) | | | | | | | | |
| OC3SNGn.6397-360a_at | Expression | Sense | 11 | ENSG00000115414 | FN1 | 2335 | fibronectin 1 | Chr 2 | Reverse | 226 |
| | probeset | (Fully Exonic) | | | | | [Source: HGNC Symbol; Acc: 3778] | | Strand | |
| OC3SNGnh.14004_at | Expression | Sense | 7 | ENSG00000115414 | FN1 | 2335 | fibronectin 1 | Chr 2 | Reverse | 227 |
| | probeset | (includes | | | | | [Source: HGNC Symbol; Acc: 3778] | | Strand | |
| | | Intronic) | | | | | | | | |
| OC3SNGnh.14004_x_at | Expression | Sense | 8 | ENSG00000115414 | FN1 | 2335 | fibronectin 1 | Chr 2 | Reverse | 228 |
| | probeset | (includes | | | | | [Source: HGNC Symbol; Acc: 3778] | | Strand | |
| | | Intronic) | | | | | | | | |
| OC3SNGnh.4044_x_at | Expression | Sense | 8 | ENSG00000115414 | FN1 | 2335 | fibronectin 1 | Chr 2 | Reverse | 229 |
| | probeset | (includes | | | | | [Source: HGNC Symbol; Acc: 3778] | | Strand | |
| | | Intronic) | | | | | | | | |
| OC3SNGnh.5967_at | Expression | Sense | 9 | ENSG00000115414 | FN1 | 2335 | fibronectin 1 | Chr 2 | Reverse | 230 |
| | probeset | (includes | | | | | [Source: HGNC Symbol; Acc: 3778] | | Strand | |
| | | Intronic) | | | | | | | | |
| OC3SNGnh.9261_at | Expression | Sense | 11 | ENSG00000115414 | FN1 | 2335 | fibronectin 1 | Chr 2 | Reverse | 231 |
| | probeset | (includes | | | | | [Source: HGNC Symbol; Acc: 3778] | | Strand | |
| | | Intronic) | | | | | | | | |
| OCADA.1039_s_at | Expression | Sense | 11 | ENSG00000115414 | FN1 | 2335 | fibronectin 1 | Chr 2 | Reverse | 232 |
| | probeset | (Fully Exonic) | | | | | [Source: HGNC Symbol; Acc: 3778] | | Strand | |
| OCADA.7873_s_at | Expression | Sense | 11 | ENSG00000115414 | FN1 | 2335 | fibronectin 1 | Chr 2 | Reverse | 233 |
| | probeset | (includes | | | | | [Source: HGNC Symbol; Acc: 3778] | | Strand | |
| | | Intronic) | | | | | | | | |
| OCEM.2081_at | Expression | Sense | 11 | ENSG00000115414 | FN1 | 2335 | fibronectin 1 | Chr 2 | Reverse | 234 |
| | probeset | (Fully Exonic) | | | | | [Source: HGNC Symbol; Acc: 3778] | | Strand | |
| OCEM.2081_x_at | Expression | Sense | 11 | ENSG00000115414 | FN1 | 2335 | fibronectin 1 | Chr 2 | Reverse | 235 |
| | probeset | (Fully Exonic) | | | | | [Source: HGNC Symbol; Acc: 3778] | | Strand | |
| OCEM.2082_at | Expression | Sense | 11 | ENSG00000115414 | FN1 | 2335 | fibronectin 1 | Chr 2 | Reverse | 236 |
| | probeset | (Fully Exonic) | | | | | [Source: HGNC Symbol; Acc: 3778] | | Strand | |
| OCEM.2082_s_at | Expression | Sense | 11 | ENSG00000115414 | FN1 | 2335 | fibronectin 1 | Chr 2 | Reverse | 237 |
| | probeset | (Fully Exonic) | | | | | [Source: HGNC Symbol; Acc: 3778] | | Strand | |
| OCEM.958_at | Expression | Sense | 11 | ENSG00000115414 | FN1 | 2335 | fibronectin 1 | Chr 2 | Reverse | 238 |
| | probeset | (includes | | | | | [Source: HGNC Symbol; Acc: 3778] | | Strand | |
| | | Intronic) | | | | | | | | |
| OCEM.958_x_at | Expression | Sense | 11 | ENSG00000115414 | FN1 | 2335 | fibronectin 1 | Chr 2 | Reverse | 239 |
| | probeset | (including | | | | | [Source: HGNC Symbol; Acc: 3778] | | Strand | |
| | | Intronic) | | | | | | | | |
| OCHP.451_s_at | Expression | Sense | 11 | ENSG00000115414 | FN1 | 2335 | fibronectin 1 | Chr 2 | Reverse | 240 |
| | probeset | (Fully Exonic) | | | | | [Source: HGNC Symbol; Acc: 3778] | | Strand | |
| OCHP.470_s_at | Expression | Sense | 11 | ENSG00000115414 | FN1 | 2335 | fibronectin 1 | Chr 2 | Reverse | 241 |
| | probeset | (Fully Exonic) | | | | | [Source: HGNC Symbol; Acc: 3778] | | Strand | |
| OCMX.493.C1_s_at | Expression | Sense | 11 | ENSG00000115414 | FN1 | 2335 | fibronectin 1 | Chr 2 | Reverse | 242 |
| | probeset | (Fully Exonic) | | | | | [Source: HGNC Symbol; Acc: 3778] | | Strand | |
| OC3P.2679.C1_s_at | Expression | Sense | 11 | ENSG00000136859 | ANGPTL2 | 23452 | angiopoietin-like 2 | Chr 9 | Reverse | 243 |
| | probeset | (Fully Exonic) | | | | | [Source: HGNC Symbol; Acc: 490] | | Strand | |
| OC3P.9834.C1_s_at | Expression | Sense | 11 | ENSG00000136859 | ANGPTL2 | 23452 | angiopoietin-like 2 | Chr 9 | Reverse | 244 |
| | probeset | (Fully Exonic) | | | | | [Source: HGNC Symbol; Acc: 490] | | Strand | |
| OCADA.8226_s_at | Expression | Sense | 11 | ENSG00000136859 | ANGPTL2 | 23452 | angiopoietin-like 2 | Chr 9 | Reverse | 245 |
| | probeset | (includes | | | | | [Source: HGNC Symbol; Acc: 490] | | Strand | |
| | | Intronic) | | | | | | | | |
| OCADNP.8811_s_at | Expression | Sense | 8 | ENSG00000136859 | ANGPTL2 | 23452 | angiopoietin-like 2 | Chr 9 | Reverse | 246 |
| | probeset | (Fully Exonic) | | | | | [Source: HGNC Symbol; Acc: 490] | | Strand | |
| OC3P.7485.C1-335a_s_at | Expression | Sense | 11 | ENSG00000165474 | GJB2 | 2706 | gap junction protein, beta 2, 26 kDa | Chr 13 | Reverse | 247 |
| | probeset | (Fully Exonic) | | | | | [Source: HGNC Symbol; Acc: 4284] | | Strand | |
| OCHP.838_s_at | Expression | Sense | 11 | ENSG00000165474 | GJB2 | 2706 | gap junction protein, beta 2, 26 kDa | Chr 13 | Reverse | 248 |
| | probeset | (Fully Exonic) | | | | | [Source: HGNC Symbol; Acc: 4284] | | Strand | |
| OC3P.10944.C1_s_at | Expression | Sense | 11 | ENSG00000122641 | INHBA | 3624 | inhibin, beta A | Chr 7 | Reverse | 249 |
| | probeset | (Fully Exonic) | | | | | [Source: HGNC Symbol; Acc: 6066] | | Strand | |
| OC3SNGnh.3606_s_at | Expression | Sense | 11 | ENSG00000122641 | INHBA | 3624 | inhibin, beta A | Chr 7 | Reverse | 250 |
| | probeset | (Fully Exonic) | | | | | [Source: HGNC Symbol; Acc: 6066] | | Strand | |
| OCADNP.7618_s_at | Expression | Sense | 11 | ENSG00000122641 | INHBA | 3624 | inhibin, beta A | Chr 7 | Reverse | 251 |
| | probeset | (Fully Exonic) | | | | | [Source: HGNC Symbol; Acc: 6066] | | Strand | |
| OCEM.2108_at | Expression | Sense | 11 | ENSG00000122641 | INHBA | 3624 | inhibin, beta A | Chr 7 | Reverse | 252 |
| | probeset | (Fully Exonic) | | | | | [Source: HGNC Symbol; Acc: 6066] | | Strand | |
| OCEM.2108_x_at | Expression | Sense | 11 | ENSG00000122641 | INHBA | 3624 | inhibin, beta A | Chr 7 | Reverse | 253 |
| | probeset | (Fully Exonic) | | | | | [Source: HGNC Symbol; Acc: 6066] | | Strand | |
| OCEM.2109_at | Expression | Sense | 8 | ENSG00000122641 | INHBA | 3624 | inhibin, beta A | Chr 7 | Reverse | 254 |
| | probeset | (Fully Exonic) | | | | | [Source: HGNC Symbol; Acc: 6066] | | Strand | |
| OCEM.2109_s_at | Expression | Sense | 11 | ENSG00000122641 | INHBA | 3624 | inhibin, beta A | Chr 7 | Reverse | 255 |
| | probeset | (Fully Exonic) | | | | | [Source: HGNC Symbol; Acc: 6066] | | Strand | |
| OCRS.977_s_at | Expression | Sense | 11 | ENSG00000122641 | INHBA | 3624 | inhibin, beta A | Chr 7 | Reverse | 256 |
| | probeset | (Fully Exonic) | | | | | [Source: HGNC Symbol; Acc: 6066] | | Strand | |
| OC3P.4123.C1_s_at | Expression | Sense | 11 | ENSG00000157227 | MMP14 | 4323 | matrix metallopeptidase 14 (membrane-inserted) | Chr 14 | Forward | 257 |
| | probeset | (Fully Exonic) | | | | | [Source: HGNC Symbol; Acc: 7160] | | Strand | |
| OC3P.4123.C1_x_at | Expression | Sense | 9 | ENSG00000157227 | MMP14 | 4323 | matrix metallopeptidase 14 (membrane-inserted) | Chr 14 | Forward | 258 |
| | probeset | (Fully Exonic) | | | | | [Source: HGNC Symbol; Acc: 7160] | | Strand | |
| OCHP.228_s_at | Expression | Sense | 11 | ENSG00000157227 | MMP14 | 4323 | matrix metallopeptidase 14 (membrane-inserted) | Chr 14 | Forward | 259 |
| | probeset | (Fully Exonic) | | | | | [Source: HGNC Symbol; Acc: 7160] | | Strand | |
| OCADNP.8653_s_at | Expression | Sense | 10 | ENSG00000122861 | PLAU | 5328 | plasminogen activator, urokinase | Chr 10 | Forward | 260 |
| | probeset | (Fully Exonic) | | | | | [Source: HGNC Symbol; Acc: 9052] | | Strand | |
| OCHP.739_s_at | Expression | Sense | 11 | ENSG00000122861 | PLAU | 5328 | plasminogen activator, urokinase | Chr 10 | Forward | 261 |
| | probeset | (Fully Exonic) | | | | | [Source: HGNC Symbol; Acc: 9052] | | Strand | |
| OC3P.11604.C1_s_at | Expression | Sense | 9 | ENSG00000137801 | THBS1 | 7057 | thrombospondin 1 | Chr 15 | Forward | 262 |
| | probeset | (Fully Exonic) | | | | | [Source: HGNC Symbol; Acc: 11785] | | Strand | |
| OC3P.9115.C1-992a_s_at | Expression | Sense | 11 | ENSG00000137801 | THBS1 | 7057 | thrombospondin 1 | Chr 15 | Forward | 263 |
| | probeset | (Fully Exonic) | | | | | [Source: HGNC Symbol; Acc: 11785] | | Strand | |
| OCADNP.4824_s_at | Expression | Sense | 11 | ENSG00000137801 | THBS1 | 7057 | thrombospondin 1 | Chr 15 | Forward | 264 |
| | probeset | (Fully Exonic) | | | | | [Source: HGNC Symbol; Acc: 11785] | | Strand | |
| OCADNP.6208_s_at | Expression | Sense | 11 | ENSG00000137801 | THBS1 | 7057 | thrombospondin 1 | Chr 15 | Forward | 265 |
| | probeset | (Fully Exonic) | | | | | [Source: HGNC Symbol; Acc: 11785] | | Strand | |
| OCHP.168_x_at | Expression | Sense | 11 | ENSG00000137801 | THBS1 | 7057 | thrombospondin 1 | Chr 15 | Forward | 266 |
| | probeset | (Fully Exonic) | | | | | [Source: HGNC Symbol; Acc: 11785] | | Strand | |
| OCMX.2515.C1_s_at | Expression | Sense | 9 | ENSG00000137801 | THBS1 | 7057 | thrombospondin 1 | Chr 15 | Forward | 267 |
| | probeset | (Fully Exonic) | | | | | [Source: HGNC Symbol; Acc: 11785] | | Strand | |
| OCMX.318.C1_s_at | Expression | Sense | 9 | ENSG00000137801 | THBS1 | 7057 | thrombospondin 1 | Chr 15 | Forward | 268 |
| | probeset | (Fully Exonic) | | | | | [Source: HGNC Symbol; Acc: 11785] | | Strand | |
| OC3P.4296.C1-409a_s_at | Expression | Sense | 11 | ENSG00000186340 | THBS2 | 7058 | thrombospondin 2 | Chr 6 | Reverse | 269 |
| | probeset | (Fully Exonic) | | | | | [Source: HGNC Symbol; Acc: 11786] | | Strand | |
| OC3P.4296.C2_s_at | Expression | Sense | 11 | ENSG00000186340 | THBS2 | 7058 | thrombospondin 2 | Chr 6 | Reverse | 270 |
| | probeset | (Fully Exonic) | | | | | [Source: HGNC Symbol; Acc: 11786] | | Strand | |
| OC3SNGn.1873-1656a_s_at | Expression | Sense | 11 | ENSG00000186340 | THBS2 | 7058 | thrombospondin 2 | Chr 6 | Reverse | 271 |
| | probeset | (Fully Exonic) | | | | | [Source: HGNC Symbol; Acc: 11786] | | Strand | |
| OC3SNGnh.14530_at | Expression | Sense | 11 | ENSG00000186340 | THBS2 | 7058 | thrombospondin 2 | Chr 6 | Reverse | 272 |
| | probeset | (includes | | | | | [Source: HGNC Symbol; Acc: 11786] | | Strand | |
| | | Intronic) | | | | | | | | |
| OC3SNGnh.14530_s_at | Expression | Sense | 11 | ENSG00000186340 | THBS2 | 7058 | thrombospondin 2 | Chr 6 | Reverse | 273 |
| | probeset | (includes | | | | | [Source: HGNC Symbol; Acc: 11786] | | Strand | |
| | | Intronic) | | | | | | | | |
| OCHP.677_s_at | Expression | Sense | 11 | ENSG00000186340 | THBS2 | 7058 | thrombospondin 2 | Chr 6 | Reverse | 274 |
| | probeset | (Fully Exonic) | | | | | [Source: HGNC Symbol; Acc: 11786] | | Strand | |
| OC3SNGnh.16386_at | Expression | Sense | 10 | ENSG00000131459 | GFPT2 | 9945 | glutamine-fructose-6-phosphate transaminase 2 | Chr 5 | Reverse | 275 |
| | probeset | (includes | | | | | [Source: HGNC Symbol; Acc: 4242] | | Strand | |
| | | Intronic) | | | | | | | | |
| OCADA.12319_s_at | Expression | Sense | 11 | ENSG00000131459 | GFPT2 | 9945 | glutamine-fructose-6-phosphate transaminase 2 | Chr 5 | Reverse | 276 |
| | probeset | (includes | | | | | [Source: HGNC Symbol; Acc: 4242] | | Strand | |
| | | Intronic) | | | | | | | | |
| OCHP.202_s_at | Expression | Sense | 11 | ENSG00000131459 | GFPT2 | 9945 | glutamine-fructose-6-phosphate transaminase 2 | Chr 5 | Reverse | 277 |
| | probeset | (Fully Exonic) | | | | | [Source: HGNC Symbol; Acc: 4242] | | Strand | |

By “biomarker signature” is meant an identifier comprised of one or more biomarkers (such as a DNA or RNA sequence, a protein or other biological molecule, a cell etc.). The expression level of the one or more biomarkers is measured and the measured expression levels allow the sample to be defined as signature positive or signature negative. Thus, at its simplest, an increased level of expression of one or more biomarkers defines a sample as positive for the biomarker signature. For certain biomarkers, a decreased level of expression of one or more biomarkers defines a sample as positive for the biomarker signature. However, where the expression level of a plurality of biomarkers is measured, the combination of expression levels is typically aggregated in order to determine whether the sample is positive for the biomarker signature. Thus, some biomarkers may display increased expression and some biomarkers may display decreased expression. This can be achieved in various ways, as discussed in detail herein.

In a general sense, in some embodiments, the biomarker signature may be considered as indicative of a particular biological state (such as the presence of a disease condition or developmental state or belonging to a particular biological subgroup). “Positive” for a biomarker signature thus may be interpreted to mean that the sample reflects the relevant biological state that the biomarker signature identifies. Similarly, “negative” for a biomarker signature means that the sample is not in (or reflective of) the relevant biological state. In the present invention, the biological state indicated by the biomarker signature is a molecular subgroup of cancer characterised by misregulation of the MAPK signalling pathway and the epithelial-mesenchymal transition (EMT) pathway. Thus, the cancer identified by the signature may have increased MAPK signalling. The cancer identified by the signature may have increased expression of both immune response and angiogenesis/vascular development genes. The cancer identified by the signature may display higher expression of EMT associated genes. This may include increased expression of VIMENTIN, AXL, TWIST1, SNAIL and/or SLUG. The increased signalling or expression is as compared to other cancers of the same type. So, for example, the cancer may be an ovarian cancer and the subgroup displays increased signalling or expression as compared to other ovarian cancers. Genes defining the EMT/Angio-Immune/MAPK pathway molecular subgroup of cancer are listed in Tables 9 and 10 below. The expression level of the genes may be measured using the probesets in Table 11. In Table 9 up-regulation and down-regulation are presented relative to gene expression levels in the overall sample set.

The biomarker signature is also correlated with particular end points as discussed in detail herein. The biomarker signature may permit selection of appropriate therapeutic interventions for example.

According to all aspects of the invention assessing whether the sample is positive or negative for the biomarker signature may comprise use of classification trees.

According to all aspects of the invention assessing whether the sample is positive or negative for the biomarker signature may comprise:
determining a sample expression score for the biomarker(s);
comparing the sample expression score to a threshold score; and
determining whether the sample expression score is above, equal to, or below the threshold expression score,
wherein if the sample expression score is above or equal to the threshold expression score the sample is positive for the biomarker signature and/or if the sample expression score is below the threshold score the sample is negative for the biomarker signature.

The skilled person will be aware that threshold expression scores may be set in a number of ways, as discussed in greater detail herein below, for example in order to maximise sensitivity and/or specificity. Thus, the sample expression score and threshold score may also be determined such that if the sample expression score is below or equal to the threshold expression score the sample is positive for the biomarker signature and/or if the sample expression score is above the threshold score the sample is negative for the biomarker signature.

“Expression levels” of biomarkers may be numerical values or directions of expression. By “directions” is meant increased or decreased expression, which may be determined as against a control or threshold expression level as explained further herein.

In the methods the sample expression score (or “signature score”) may be derived according to the formula:

$$SignatureScore = {\sum }_{i} {w}_{i} \times ( {ge}_{i} - {b}_{i} ) + k$$

Where wᵢ is a weight for each gene, bᵢ is a gene-specific bias, geᵢ is the gene expression after pre-processing, and k is a constant offset.

The sample expression score may be derived using the expression level(s) of any of the genes or groups of genes described herein. The sample expression score may be derived using the expression level of one or more additional genes.

According to all aspects of the invention the expression score may be calculated using a weight value and a bias value for each biomarker. For example, the weight value and the bias value may be as defined for each biomarker in Table A and/or Table B. The expression score may be calculated using a weight value for each biomarker.

As used herein, the term “weight” refers to the absolute magnitude of an item in a mathematical calculation. The weight of each biomarker in a gene expression classifier or signature may be determined on a data set of patient samples using learning methods known in the art. As used herein the term “bias” or “offset” refers to a constant term derived using the mean expression of the signatures genes in a training set and is used to mean-center each gene analyzed in the test dataset.

By “expression score” is meant a compound decision score that summarizes the expression levels of the biomarkers. This may be compared to a threshold score that is mathematically derived from a training set of patient data. The threshold score is established with the purpose of maximizing the ability to separate cancers into those that are positive for the biomarker signature and those that are negative. The patient training set data is preferably derived from cancer tissue samples having been characterized by sub-type, prognosis, likelihood of recurrence, long term survival, clinical outcome, treatment response, diagnosis, cancer classification, or personalized genomics profile. Expression profiles, and corresponding decision scores from patient samples may be correlated with the characteristics of patient samples in the training set that are on the same side of the mathematically derived score decision threshold. In certain example embodiments, the threshold of the (linear) classifier scalar output is optimized to maximize the sum of sensitivity and specificity under cross-validation as observed within the training dataset.

The overall expression data for a given sample may be normalized using methods known to those skilled in the art in order to correct for differing amounts of starting material, varying efficiencies of the extraction and amplification reactions, etc.

In one embodiment, the biomarker expression levels in a sample are evaluated by a (linear) classifier. As used herein, a (linear) classifier refers to a weighted sum of the individual biomarker intensities into a compound decision score (“decision function”). The decision score is then compared to a pre-defined cut-off score threshold, corresponding to a certain set-point in terms of sensitivity and specificity which indicates if a sample is equal to or above the score threshold (decision function positive) or below (decision function negative).

Using a (linear) classifier on the normalized data to make a call (e.g. positive or negative for a biomarker signature) effectively means to split the data space, i.e. all possible combinations of expression values for all genes in the classifier, into two disjoint segments by means of a separating hyperplane. This split is empirically derived on a (large) set of training examples. Without loss of generality, one can assume a certain fixed set of values for all but one biomarker, which would automatically define a threshold value for this remaining biomarker where the decision would change from, for example, positive or negative for the biomarker signature. The precise value of this threshold depends on the actual measured expression profile of all other biomarkers within the classifier, but the general indication of certain biomarkers remains fixed. Therefore, in the context of the overall gene expression classifier, relative expression can indicate if either up- or down-regulation of a certain biomarker is indicative of being positive for the signature or not. In certain example embodiments, a sample expression score above the threshold expression score indicates the sample is positive for the biomarker signature. In certain other example embodiments, a sample expression score above a threshold score indicates the subject has a poor clinical prognosis compared to a subject with a sample expression score below the threshold score.

In certain other example embodiments, the expression signature is derived using a decision tree (Hastie et al. The Elements of Statistical Learning, Springer, New York 2001), a random forest (Breiman, 2001 Random Forests, Machine Learning 45:5), a neural network (Bishop, Neural Networks for Pattern Recognition, Clarendon Press, Oxford 1995), discriminant analysis (Duda et al. Pattern Classification, 2nd ed., John Wiley, New York 2001), including, but not limited to linear, diagonal linear, quadratic and logistic discriminant analysis, a Prediction Analysis for Microarrays (PAM, (Tibshirani et al., 2002, Proc. Natl. Acad. Sci. USA 99:6567-6572)) or a Soft Independent Modeling of Class Analogy analysis. (SIMCA, (Wold, 1976, Pattern Recogn. 8:127-139)). Classification trees (Breiman, Leo; Friedman, J. H.; Olshen, R. A.; Stone, C. J. (1984). Classification and regression trees. Monterey, Calif.: Wadsworth & Brooks/Cole Advanced Books & Software. ISBN 978-0-412-04841-8) provide a means of predicting outcomes based on logic and rules. A classification tree is built through a process called binary recursive partitioning, which is an iterative procedure of splitting the data into partitions/branches. The goal is to build a tree that distinguishes among pre-defined classes. Each node in the tree corresponds to a variable. To choose the best split at a node, each variable is considered in turn, where every possible split is tried and considered, and the best split is the one which produces the largest decrease in diversity of the classification label within each partition. This is repeated for all variables, and the winner is chosen as the best splitter for that node. The process is continued at the next node and in this manner, a full tree is generated. One of the advantages of classification trees over other supervised learning approaches such as discriminant analysis, is that the variables that are used to build the tree can be either categorical, or numeric, or a mix of both. In this way it is possible to generate a classification tree for predicting outcomes based on say the directionality of gene expression. Random forest algorithms (Breiman, Leo (2001). “Random Forests”. Machine Learning 45 (1): 5-32. doi:10.1023/A:1010933404324) provide a further extension to classification trees, whereby a collection of classification trees are randomly generated to form a “forest” and an average of the predicted outcomes from each tree is used to make inference with respect to the outcome.

Biomarker expression values may be defined in combination with corresponding scalar weights on the real scale with varying magnitude, which are further combined through linear or non-linear, algebraic, trigonometric or correlative means into a single scalar value via an algebraic, statistical learning, Bayesian, regression, or similar algorithms which together with a mathematically derived decision function on the scalar value provide a predictive model by which expression profiles from samples may be resolved into discrete classes of responder or non-responder, resistant or non-resistant, to a specified drug, drug class, molecular subtype, or treatment regimen. Such predictive models, including biomarker membership, are developed by learning weights and the decision threshold, optimized for sensitivity, specificity, negative and positive predictive values, hazard ratio or any combination thereof, under cross-validation, bootstrapping or similar sampling techniques, from a set of representative expression profiles from historical patient samples with known drug response and/or resistance.

In one embodiment, the biomarkers are used to form a weighted sum of their signals, where individual weights can be positive or negative. The resulting sum (“expression score”) is compared with a pre-determined reference point or value. The comparison with the reference point or value may be used to diagnose, or predict a clinical condition or outcome.

As described above, one of ordinary skill in the art will appreciate that the biomarkers included in the classifier provided in Table A and/or Table B will carry unequal weights in a classifier. Therefore, while as few as one biomarker may be used to diagnose or predict a clinical prognosis or response to a therapeutic agent, the specificity and sensitivity or diagnosis or prediction accuracy may increase using more biomarkers.

In certain example embodiments, the expression signature is defined by a decision function. A decision function is a set of weighted expression values derived using a (linear) classifier. All linear classifiers define the decision function using the following equation:

f(x)=w′·x+b=Σwᵢ·xᵢ+b (1)

All measurement values, such as the microarray gene expression intensities xᵢ, for a certain sample are collected in a vector x. Each intensity is then multiplied with a corresponding weight wᵢ to obtain the value of the decision function f(x) after adding an offset term b. In deriving the decision function, the linear classifier will further define a threshold value that splits the gene expression data space into two disjoint sections. Example (linear) classifiers include but are not limited to partial least squares (PLS), (Nguyen et al., Bioinformatics 18 (2002) 39-50), support vector machines (SVM) (Schölkopf et al., Learning with Kernels, MIT Press, Cambridge 2002), and shrinkage discriminant analysis (SDA) (Ahdesmäki et al., Annals of applied statistics 4, 503-519 (2010)). In one example embodiment, the (linear) classifier is a PLS linear classifier.

The decision function is empirically derived on a large set of training samples, for example from patients showing a good or poor clinical prognosis. The threshold separates a patient group based on different characteristics such as, but not limited to, clinical prognosis before or after a given therapeutic treatment. The interpretation of this quantity, i.e. the cut-off threshold, is derived in the development phase (“training”) from a set of patients with known outcome. The corresponding weights and the responsiveness/resistance cut-off threshold for the decision score are fixed a priori from training data by methods known to those skilled in the art. In one example embodiment, Partial Least Squares Discriminant Analysis (PLS-DA) is used for determining the weights. (L. Ståhle, S. Wold, J. Chemom. 1 (1987) 185-196; D. V. Nguyen, D. M. Rocke, Bioinformatics 18 (2002) 39-50).

Effectively, this means that the data space, i.e. the set of all possible combinations of biomarker expression values, is split into two mutually exclusive groups corresponding to different clinical classifications or predictions, for example, one corresponding to good clinical prognosis and poor clinical prognosis. In the context of the overall classifier, relative over-expression of a certain biomarker can either increase the decision score (positive weight) or reduce it (negative weight) and thus contribute to an overall decision of, for example, a good clinical prognosis.

In certain example embodiments of the invention, the data is transformed non-linearly before applying a weighted sum as described above. This non-linear transformation might include increasing the dimensionality of the data. The non-linear transformation and weighted summation might also be performed implicitly, for example, through the use of a kernel function. (Schölkopf et al. Learning with Kernels, MIT Press, Cambridge 2002).

In certain example embodiments, the patient training set data is derived by isolated RNA from a corresponding cancer tissue sample set and determining expression values by hybridizing the (cDNA amplified from) isolated RNA to a microarray. In certain example embodiments, the microarray used in deriving the expression signature is a transcriptome array. As used herein a “transcriptome array” refers to a microarray containing probe sets that are designed to hybridize to sequences that have been verified as expressed in the diseased tissue of interest. Given alternative splicing and variable poly-A tail processing between tissues and biological contexts, it is possible that probes designed against the same gene sequence derived from another tissue source or biological context will not effectively bind to transcripts expressed in the diseased tissue of interest, leading to a loss of potentially relevant biological information. Accordingly, it is beneficial to verify what sequences are expressed in the disease tissue of interest before deriving a microarray probe set. Verification of expressed sequences in a particular disease context may be done, for example, by isolating and sequencing total RNA from a diseased tissue sample set and cross-referencing the isolated sequences with known nucleic acid sequence databases to verify that the probe set on the transcriptome array is designed against the sequences actually expressed in the diseased tissue of interest. Methods for making transcriptome arrays are described in United States Patent Application Publication No. 2006/0134663, which is incorporated herein by reference. In certain example embodiments, the probe set of the transcriptome array is designed to bind within 300 nucleotides of the 3′ end of a transcript. Methods for designing transcriptome arrays with probe sets that bind within 300 nucleotides of the 3′ end of target transcripts are disclosed in United States Patent Application Publication No. 2009/0082218, which is incorporated by reference herein. In certain example embodiments, the microarray used in deriving the gene expression profiles of the present invention is the Almac Ovarian Cancer DSA™ microarray (Almac Group, Craigavon, United Kingdom).

An optimal (linear) classifier can be selected by evaluating a (linear) classifier's performance using such diagnostics as “area under the curve” (AUC). AUC refers to the area under the curve of a receiver operating characteristic (ROC) curve, both of which are well known in the art. AUC measures are useful for comparing the accuracy of a classifier across the complete data range. (Linear) classifiers with a higher AUC have a greater capacity to classify unknowns correctly between two groups of interest (e.g., ovarian cancer samples and normal or control samples). ROC curves are useful for plotting the performance of a particular feature (e.g., any of the biomarkers described herein and/or any item of additional biomedical information) in distinguishing between two populations (e.g., individuals responding and not responding to a therapeutic agent). Typically, the feature data across the entire population (e.g., the cases and controls) are sorted in ascending order based on the value of a single feature. Then, for each value for that feature, the true positive and false positive rates for the data are calculated. The true positive rate is determined by counting the number of cases above the value for that feature and then dividing by the total number of positive cases. The false positive rate is determined by counting the number of controls above the value for that feature and then dividing by the total number of controls. Although this definition refers to scenarios in which a feature is elevated in cases compared to controls, this definition also applies to scenarios in which a feature is lower in cases compared to the controls (in such a scenario, samples below the value for that feature would be counted). ROC curves can be generated for a single feature as well as for other single outputs, for example, a combination of two or more features can be mathematically combined (e.g., added, subtracted, multiplied, etc.) to provide a single sum value, and this single sum value can be plotted in a ROC curve. Additionally, any combination of multiple features, in which the combination derives a single output value, can be plotted in a ROC curve. These combinations of features may comprise a test. The ROC curve is the plot of the true positive rate (sensitivity) of a test against the false positive rate (1-specificity) of the test.

Alternatively, an optimal classifier can be selected by evaluating performance against time-to-event endpoints using methods such as Cox proportional hazards (PH) and measures of performance across all possible thresholds assessed via the concordance-index (C-index) (Harrell, Jr. 2010). The C-Index is analagous to the “area under the curve” (AUC) metric (used for dichotomised endpoints), and it is used to measure performance with respect to association with survival data. Note that the extension of AUC to time-to-event endpoints is the C-index, with threshold selection optimised to maximise the hazard ratio (HR) under cross-validation. In this instance, the partial Cox regression algorithm (Li and Gui, 2004) was chosen for the biomarker discovery analyses. It is analogous to principal components analysis in that the first few latent components explain most of the information in the data. Implementation is as described in Ahdesmaki et al 2013.

C-index values can be generated for a single feature as well as for other single outputs, for example, a combination of two or more features can be mathematically combined (e.g., added, subtracted, multiplied, etc.) to provide a single sum value, and this single sum value can be evaluated for statistical significance. Additionally, any combination of multiple features, in which the combination derives a single output value, can be evaluated as a C-index for assessing utility for time-to-event class separation. These combinations of features may comprise a test. The C-index (Harrell, Jr. 2010, see Equation 4) of the continuous cross-validation test set risk score predictions was evaluated as the main performance measure.

In one example embodiment an expression signature is directed to the biomarkers detailed in Table A and/or Table B with corresponding ranks, and weights and associated bias detailed in the tables or alternative rankings, and weightings and bias, depending, for example, on the disease setting. The methods of the invention may rely upon measuring one or more, up to all, of the biomarkers listed in Table A and/or Table B (optionally together with one or more additional biomarkers).

The invention provides for patient selection for therapy and thus may contribute to improved outcomes in response to particular classes of therapy. Accordingly, the invention also relates to a method of treating cancer comprising administering a MAPK pathway inhibitor, an EMT pathway inhibitor, an SRC pathway inhibitor, an anti-angiogenic therapeutic agent, a taxane and/or a platinum-based chemotherapeutic agent to a subject wherein the subject is selected for treatment on the basis of a method as described herein.

In a related aspect, the present invention provides a method of treating cancer comprising administering a therapeutic agent to a subject wherein the subject is selected for treatment by
(i) measuring the expression level(s) of at least 1 biomarker(s) selected from Table A or Table B in a sample from the subject;
(ii) assessing from the expression level(s) of the at least 1 biomarker(s) whether the sample from the subject is positive or negative for a biomarker signature comprising the at least 1 biomarker, wherein
(a) if the sample is positive for the biomarker signature the therapeutic agent is a MAPK pathway inhibitor; and/or
(b) if the sample is positive for the biomarker signature the therapeutic agent is an EMT pathway inhibitor; and/or
(c) if the sample is negative for the biomarker signature the therapeutic agent is an SRC pathway inhibitor; and/or
(d) if the sample is negative for the biomarker signature the therapeutic agent is a platinum-based chemotherapeutic agent; and/or
(e) if the sample is positive for the biomarker signature the therapeutic agent is a taxane.

In a further aspect the invention provides a method of treating cancer comprising administering a therapeutic agent to a subject wherein the subject is selected for treatment by
(i) measuring the expression level(s) of at least COL5A1 and/or THBS1 in a sample from the subject;
(ii) assessing from the expression level(s) of at least COL5A1 and/or THBS1 whether the sample from the subject is positive or negative for a biomarker signature comprising COL5A1 and/or THBS1, wherein if the sample is positive for the biomarker signature the therapeutic agent is an anti-angiogenic therapeutic agent.

The invention also relates to a MAPK pathway inhibitor, an EMT pathway inhibitor, an SRC pathway inhibitor, an anti-angiogenic therapeutic agent, a taxane and/or a platinum-based chemotherapeutic agent for use in treating cancer in a subject, wherein the subject is selected for treatment on the basis of a method as described herein.

In yet a further related aspect, the present invention provides a therapeutic agent for use in treating cancer in a subject wherein the subject is selected for treatment by:
(i) measuring the expression level(s) of at least 1 biomarker(s) selected from Table A or Table B in a sample from the subject;
(ii) assessing from the expression level(s) of the at least 1 biomarker(s) whether the sample from the subject is positive or negative for a biomarker signature comprising the at least 1 biomarker, wherein:
(a) if the sample is positive for the biomarker signature the therapeutic agent is a MAPK pathway inhibitor; and/or
(b) if the sample is positive for the biomarker signature the therapeutic agent is an EMT pathway inhibitor; and/or
(c) if the sample is negative for the biomarker signature the therapeutic agent is an SRC pathway inhibitor; and/or
(d) if the sample is negative for the biomarker signature the therapeutic agent is a platinum-based chemotherapeutic agent; and/or
(e) if the sample is positive for the biomarker signature the therapeutic agent is a taxane.

According to a further aspect of the invention there is provided a therapeutic agent for use in treating cancer in a subject wherein the subject is selected for treatment by:
(i) measuring the expression level(s) of at least COL5A1 and/or THBS1 in a sample from the subject;
(ii) assessing from the expression level(s) of at least COL5A1 and/or THBS1 whether the sample from the subject is positive or negative for a biomarker signature comprising COL5A1 and/or THBS1, wherein if the sample is positive for the biomarker signature the therapeutic agent is an anti-angiogenic therapeutic agent.

According to a further aspect of the invention there is provided a method of treating cancer comprising administering a therapeutic agent to a subject wherein
(a) if the subject is positive for a biomarker signature comprising the expression level(s) of at least 1 biomarker(s) selected from Table A or Table B the therapeutic agent is a MAPK pathway inhibitor; and/or
(b) if the subject is positive for the biomarker signature comprising the expression level(s) of at least 1 biomarker(s) selected from Table A or Table B the therapeutic agent is an EMT pathway inhibitor; and/or
(c) if the subject is negative for the biomarker signature comprising the expression level(s) of at least 1 biomarker(s) selected from Table A or Table B the therapeutic agent is an SRC pathway inhibitor; and/or
(d) if the subject is negative for the biomarker signature comprising the expression level(s) of at least 1 biomarker(s) selected from Table A or Table B the therapeutic agent is a platinum-based chemotherapeutic agent; and/or
(e) if the subject is positive for a biomarker signature comprising the expression level(s) of at least 1 biomarker(s) selected from Table A or Table B the therapeutic agent is a taxane.

Also provided is a method of treating cancer comprising administering a therapeutic agent to a subject wherein if the subject is positive for a biomarker signature comprising the expression level(s) of at least COL5A1 and/or THBS1 the therapeutic agent is an anti-angiogenic therapeutic agent.

The invention also relates to a therapeutic agent for use in treating cancer in a subject, wherein
(a) if the sample is positive for a biomarker signature comprising the expression level(s) of at least 1 biomarker(s) selected from Table A or Table B the therapeutic agent is a MAPK pathway inhibitor; and/or
(b) if the sample is positive for the biomarker signature comprising the expression level(s) of at least 1 biomarker(s) selected from Table A or Table B the therapeutic agent is an EMT pathway inhibitor; and/or
(c) if the sample is negative for the biomarker signature comprising the expression level(s) of at least 1 biomarker(s) selected from Table A or Table B the therapeutic agent is an SRC pathway inhibitor; and/or
(d) if the sample is negative for the biomarker signature comprising the expression level(s) of at least 1 biomarker(s) selected from Table A or Table B the therapeutic agent is a platinum-based chemotherapeutic agent; and/or
(e) if the sample is positive for a biomarker signature comprising the expression level(s) of at least 1 biomarker(s) selected from Table A or Table B the therapeutic agent is a taxane.

Also provided is a therapeutic agent for use in treating cancer in a subject, wherein if the sample is positive for a biomarker signature comprising the expression level(s) of at least COL5A1 and/or THBS1 the therapeutic agent is an anti-angiogenic therapeutic agent.

In yet a further aspect, the present invention relates to a method of treating cancer comprising administering a therapeutic agent to a subject, wherein:
(a) the subject has been identified as having an EMT cancer and the therapeutic agent is a MAPK pathway inhibitor; and/or
(b) the subject has been identified as having an EMT cancer and the therapeutic agent is an EMT pathway inhibitor; and/or
(c) the subject has been identified as having a non-EMT cancer and the therapeutic agent is an SRC pathway inhibitor; and/or
(d) the subject has been identified as having a non-EMT cancer and the therapeutic agent is a platinum-based chemotherapeutic agent; and/or
(e) the subject has been identified as having an EMT cancer and the therapeutic agent is a taxane.

According to a further aspect of the invention there is provided a therapeutic agent for use in treating cancer in a subject, wherein
(a) the subject has been identified as having an EMT cancer and the therapeutic agent is a MAPK pathway inhibitor; and/or
(b) the subject has been identified as having an EMT cancer and the therapeutic agent is an EMT pathway inhibitor; and/or
(c) the subject has been identified as having a non-EMT cancer and the therapeutic agent is an SRC pathway inhibitor; and/or
(d) the subject has been identified as having a non-EMT cancer and the therapeutic agent is a platinum-based chemotherapeutic agent; and/or
(e) the subject has been identified as having an EMT cancer and the therapeutic agent is a taxane.

By “EMT cancer” is meant a cancer falling within the molecular subgroup identified by the present inventors, which is detectable using the biomarker signatures of the invention and described herein, for example based on the expression levels of one or more biomarkers from Tables A and B. The cancer may thus display epithelial-mesenchymal transition (EMT), which may contribute to angiogenic processes and disease progression. An EMT cancer can also be termed an Angio-Immune cancer or a MAPK pathway (MEK) cancer in view of the contributing pathways to the subgroup. Genes defining the EMT/Angio-Immune/MAPK pathway molecular subgroup of cancer are listed in Tables 9 and 10 below. In Table 9 up-regulation and down-regulation are presented relative to gene expression levels in the overall sample set.

According to all aspects of the invention the therapeutic agent may be a MAPK pathway inhibitor combined with a platinum-based chemotherapeutic agent and/or an SRC pathway inhibitor.

The invention also relates to a method of treating cancer comprising administering a combination of a platinum-based chemotherapeutic agent and a MAPK pathway inhibitor, wherein:
(a) the combination is used as a first line treatment; or
(b) the combination is used for a cancer identified as resistant to a platinum-based chemotherapeutic agent.

In a further aspect, the present invention relates to a combination of a platinum-based chemotherapeutic agent and a MAPK pathway inhibitor for use in a method of treating cancer, wherein:
(a) the combination is used as a first line treatment; or
(b) the combination is used for a cancer identified as resistant to a platinum-based chemotherapeutic agent.

According to all relevant aspects of the invention, the platinum-based chemotherapeutic agent and the MAPK pathway inhibitor may be administered together and/or sequentially in time in either order.

According to all aspects of the invention, a therapeutic agent may be a chemically synthesized pharmaceutical, a biologic, vaccine or small molecule. Biologics include antibodies and derivatives thereof as discussed further herein, recombinant therapeutic proteins, sugars and nucleic acids.

By “MAPK pathway inhibitor” is meant a therapeutic agent, such as a pharmaceutical drug, that inhibits signalling via the MAPK pathway. The inhibitor may be specific for the MAPK pathway. Thus, in certain embodiments the MAPK pathway inhibitor is not a multi-pathway inhibitor. In further embodiments the MAPK pathway inhibitor is a RAS/RAF/MEK/ERK pathway inhibitor. In specific embodiments the MAPK pathway inhibitor is a (specific) RAS, RAF, MEK and/or MAPK inhibitor. By MEK inhibitor is meant a therapeutic agent, such as a pharmaceutical drug, that (specifically) inhibits the mitogen-activated protein kinase kinase enzymes MEK1 and/or MEK2.

In certain embodiments the MAPK pathway inhibitor is selected from Table G and/or H. In certain embodiments the MAPK pathway inhibitor (specifically) inhibits one or more of the targets listed in Table H. In specific embodiments the MAPK pathway inhibitor is trametinib. In further specific embodiments the MAPK pathway inhibitor is selumetinib (synonyms: AZD6244 and ARRY-142886).

TABLE G
MAPK pathway inhibitors
Preclinical - Phase I

| DRUG NAME | COMPANY |
|---|---|
| BAL-3833 | Basilea Pharmaceutica |
| BGB-283 | Merck KGaA |
| HM-95573 | Hanmi Pharmaceuticals |
| LY-3009120 | Eli Lilly |
| RG-7304 | Roche |
| RG-7842 | Genentech |
| Salirasib | Ono Pharmaceutical |
| AEZS-136 | Aeterna Zentaris Inc. |
| ARI-4175 | Arisaph Pharmaceuticals |
| ASN-003 | Asana BioSciences |
| CCT-196969 | Basilea Pharmaceutica |
| CCT-241161 | Basilea Pharmaceutica |
| CS-410 | Chipscreen Biosciences |
| MAP4K4 Inhibitor (small | Genentech, Inc. |
| molecule) for Cancer | |
| pan-RAF Kinase inhibitor | Novartis AG |
| (small molecule) for | |
| Oncology | |
| CT-207 | HEC Pharm Co., Ltd. |
| CT-317 | HEC Pharm Co., Ltd. |
| B-Raf Kinase inhibitor | Ruga Corporation |
| for Cancer | |
| EBI-907 | Eternity Bioscience Inc. |
| EBI-945 | Eternity Bioscience Inc. |
| KO-947 | Kura Oncology, Inc. |
| LXH-254 | Novartis AG |
| MDC-1016 | Medicon Pharmaceuticals, Inc |
| MT-477 | Medisyn Technologies, Inc. |
| NCB-0594 | Carna Biosciences, Inc. |
| NCB-0846 | Carna Biosciences, Inc. |
| NMSP-285 | Nerviano Medical Sciences S.r.l. |
| ON-108600 | Onconova Therapeutics, Inc. |
| PV-103 | PepVax, Inc. |
| RX-8243 | Rexahn Pharmaceuticals, Inc. |
| STP-503 | Sirnaomics, Inc. |
| Raf Kinases inhibitor | Amitech Therapeutic Solutions, Inc. |
| (small molecule) for | |
| Cancer | |
| TAK-632 | Takeda Pharmaceutical Company |
| | Limited |
| TEW-0201 | MedPacto, Inc. |
| AIK-4 | Allinky Biopharma |
| AR-00457679 | Array BioPharma Inc. |
| CB-745 | AGV Discovery, SAS |
| HD-001 | AstraZeneca Plc |
| SCH-722984 | Merck & Co., Inc. |
| K-RAS inhibitor (small | Aurigene Discovery Technologies Limited |
| molecule) for Oncology | |
| B-RAF Kinase inhibitor | Sareum Holdings Plc |
| (small molecule) for | |
| Oncology | |
| ERK2 and Aurora B | Aeterna Zentaris Inc. |
| Kinase inhibitor (small | |
| molecule) for Cancer | |
| ARQ-736 | ArQule, Inc. |
| K-Ras inhibitor (small | Boehringer Ingelheim GmbH |
| molecule) for Oncology | |
| KRAS inhibitor (small | NantBioScience |
| molecule) for Cancer | |
| KRas inhibitor (small | Nimbus Therapeutics, LLC |
| molecule) for Solid | |
| Tumor | |
| Pan-Raf Kinases inhibitor | Redx Pharma Plc |
| (small molecule) for | |
| Colorectal Cancer | |
| TNIK inhibitor (small | Astex Pharmaceuticals, Inc. |
| molecule) for Oncology | |
| KRAS inhibitor (synthetic | PeptiDream Inc. |
| peptide) for Oncology | |
| K-Ras inhibitor (small | Araxes Pharma LLC |
| molecule) for Cancer | |

Phase II, Phase III, Marketed

| DRUG NAME | COMPANY NAME | DISEASE INDICATION |
|---|---|---|
| vemurafenib (Zelboraf) | Roche | Marketed: mMelanoma |
| | | Phase II: Bile Duct, Bladder, CLL, GI, Leukemia, |
| | | Ovarian, Prostate, Sarcomas, Thyroid |
| regorafenib (Stivarga) | Bayer | Marketed: GIST, mCRC |
| | | PhIII: HCC |
| | | PhII: Bile Duct, Ovarian, Pancreatic, RCC, |
| | | Salivary Gland, Soft Tissue Sarcoma; Bladder |
| dabrafenib (Tafinlar) | Novartis | Marketed: mMelanoma |
| | | PhII: Thyroid, CRC, NSCLC, (GIST), Glioma, |
| | | Leukemia, Brain, Multiple Myeloma, Germ Cell |
| | | Tumors |
| RAF-265 | Novartis | PhII: mMelanoma |
| Encorafenib | Array Biopharma | PhIII: mMelanoma |
| | | PhII: mCRC |
| Donafenib | Suzhou Zelgen | PhII: Esophageal, GI, HCC, mCRC |
| | Biopharmaceutical | |
| NEO-100 | Neonc | PhII: Recurrent Glioblastoma Multiforme (GBM) |
| | Technologies | Preclinical: Lung |
| PLX-8394 | Plexxikon | phII: Thyroid, Bile Duct, CRC, Melanoma, |
| | | NSCLC |
| | | phI: Leukemias |
| RXDX-105 | Ingnyta | PhII: Colon Carcinoma, Melanoma, mCRC |
| TAK-580 | Millennium | PhII: Metastatic Melanoma; Solid Tumor |
| | Pharmaceuticals | PhI: Nonhematologic Malignancy |
| Ulixertinib | BioMed Valley | PhII: AML, CRC, Melanoma, Myelodysplastic |
| | Discoveries | Syndrome; NSCLC |
| | | Preclinical: Metastatic Adenocarcinoma of The |
| | | Pancreas; Pancreatic |

TABLE H
MAPK pathway inhibitors data

| | Ras Inhibitor; Raf Kinase Inhibitor; A-Raf Kinase Inhibitor; B-Raf Kinase Inhibitor; C-Raf Kinase Inhibitor; TRAF2 and | | | | | | | |
|---|---|---|---|---|---|---|---|---|
| | NCK-Interacting Protein Kinase (TNIK) Inhibitor; Mitogen Activated Protein Kinase Kinase Kinase (cMos or cRaf or | | | | | | | |
| | MAPKKK or MAP3K or MAP Kinase Kinase Kinase or EC 2.7.11.25) Inhibitor; Mitogen Activated Protein Kinase 3 | | | | | | | |
| | (MAP Kinase 3 or MAPK 3 or Extracellular Signal Regulated Kinase 1 or ERK-1) Inhibitor; Mitogen-Activated Protein | | | | | | | |
| | Kinase Kinase Kinase Kinase 2 (Germinal Center Kinase or MAPK/ERK Kinase Kinase Kinase 2) Inhibitor; Mitogen | | | | | | | |
| | Activated Protein Kinase 1 (MAP Kinase 1 or MAPK 1 or Extracellular Signal Regulated Kinase 2 or ERK-2 or | | | | | | | |
| | Protein Tyrosine Kinase ERK2 or EC 2.7.11.24) Inhibitor; Mitogen Activated Protein Kinase Kinase Kinase 5 | | | | | | | |
| | (Apoptosis Signal Regulating Kinase 1 or MAPK/ERK Kinase Kinase 5 or MEK Kinase 5 or ASK-1 or MAP3K5 or EC | | | | | | | |
| Mechanism | 2.7.11.25) Inhibitor; Mitogen-Activated Protein Kinase Kinase Kinase Kinase 4 (HPK/GCK-Like Kinase HGK or | | | | | | | |
| Of Action: | MAPK/ERK Kinase Kinase Kinase 4 or MEK Kinase Kinase 4 or MEKKK 4 or MAP4K4 or EC 2.7.11.1) Inhibitor | | | | | | | |
| Drug Name | Generic Name | Brand Name | Company | Therapy Area | Indications | Product Stage | Product Geography | MOA |
| vemurafenib | vemurafenib | Zelboraf | Chugai | Oncology | Metastatic Melanoma | Marketed | Japan | B-Raf Kinase Inhibitor |
| | | | Pharmaceutical Co., Ltd. | | | | | |
| vemurafenib | vemurafenib | Zelboraf | F. Hoffmann- | Oncology | Metastatic Melanoma | Marketed | Brazil | B-Raf Kinase Inhibitor |
| | | | La Roche Ltd. | | | | | |
| vemurafenib | vemurafenib | Zelboraf | Hoffmann- | Oncology | Metastatic Melanoma | Marketed | Canada; United States | B-Raf Kinase Inhibitor |
| | | | La Roche Inc. | | | | | |
| vemurafenib | vemurafenib | Zelboraf | Roche Korea Co. Ltd. | Oncology | Metastatic Melanoma | Marketed | South Korea | B-Raf Kinase Inhibitor |
| vemurafenib | vemurafenib | Zelboraf | Roche Pharma AG | Oncology | Metastatic Melanoma | Marketed | Germany | B-Raf Kinase Inhibitor |
| vemurafenib | vemurafenib | Zelboraf | Roche | Oncology | Metastatic Melanoma | Marketed | Australia | B-Raf Kinase Inhibitor |
| | | | Products (Pty) Ltd | | | | | |
| vemurafenib | vemurafenib | Zelboraf | Roche | Oncology | Metastatic Melanoma | Marketed | United Kingdom | B-Raf Kinase Inhibitor |
| | | | Products Limited | | | | | |
| vemurafenib | vemurafenib | Zelboraf | Roche | Oncology | Metastatic Melanoma | Marketed | EU; France; Spain | B-Raf Kinase Inhibitor |
| | | | Registration Ltd | | | | | |
| vemurafenib | vemurafenib | Zelboraf | Roche S.p.A. | Oncology | Metastatic Melanoma | Marketed | Italy | B-Raf Kinase Inhibitor |
| vemurafenib | vemurafenib | Zelboraf | F. Hoffmann- | Oncology | Metastatic Melanoma | Phase III | EU; United States | B-Raf Kinase Inhibitor |
| | | | La Roche Ltd. | | | | | |
| vemurafenib | vemurafenib | Zelboraf | F. Hoffmann- | Oncology | Bile Duct Cancer | Phase II | Global | B-Raf Kinase Inhibitor |
| | | | La Roche Ltd. | | (Cholangiocarcinoma); | | | |
| | | | | | Bladder Cancer; | | | |
| | | | | | Chronic Lymphocytic | | | |
| | | | | | Leukemia (CLL); | | | |
| | | | | | Gastrointestinal | | | |
| | | | | | Stromal Tumor | | | |
| | | | | | (GIST); Hairy Cell | | | |
| | | | | | Leukemia; Ovarian | | | |
| | | | | | Cancer; Prostate | | | |
| | | | | | Cancer; Sarcomas; | | | |
| | | | | | Thyroid Cancer | | | |
| vemurafenib | vemurafenib | Zelboraf | F. Hoffmann- | Oncology | Colorectal Cancer | Phase II | EU; United States | B-Raf Kinase Inhibitor |
| | | | La Roche Ltd. | | | | | |
| vemurafenib | vemurafenib | Zelboraf | F. Hoffmann- | Oncology | Non-Small | Phase II | Global | B-Raf Kinase Inhibitor |
| | | | La Roche Ltd. | | Cell Lung Cancer; | | | |
| | | | | | Refractory Multiple | | | |
| | | | | | Myeloma; Relapsed | | | |
| | | | | | Multiple Myeloma | | | |
| vemurafenib | vemurafenib | Zelboraf | F. Hoffmann- | Oncology | Papillary | Discontinued | EU; United States | B-Raf Kinase Inhibitor |
| | | | La Roche Ltd. | | Thyroid Cancer | | | |
| regorafenib | regorafenib | Stivarga | Bayer (Pty) Ltd. | Oncology | Gastrointestinal | Marketed | South Africa | Abl Tyrosine Kinase Inhibitor; B-Raf |
| | | | | | Stromal Tumor | | | Kinase Inhibitor; c-kit Receptor |
| | | | | | (GIST); Metastatic | | | (SCFR, CD117) Antagonist; C-Raf |
| | | | | | Colorectal Cancer | | | Kinase Inhibitor; CD167b (Discoidin |
| | | | | | | | | Domain-Containing Receptor 2 or |
| | | | | | | | | Neurotrophic Tyrosine Kinase, |
| | | | | | | | | Receptor-Related 3) Inhibitor; |
| | | | | | | | | Fibroblast Growth Factor Receptor 1 |
| | | | | | | | | (FGFR1) Antagonist; Fibroblast |
| | | | | | | | | Growth Factor Receptor 2 |
| | | | | | | | | (Keratinocyte Growth Factor Receptor |
| | | | | | | | | or CD332 or FGFR2 or KGFR or K- |
| | | | | | | | | sam or EC 2.7.10.1) Antagonist; |
| | | | | | | | | Platelet Derived Growth Factor |
| | | | | | | | | Receptor Alpha (PDGFR-Alpha) |
| | | | | | | | | Antagonist; Platelet Derived Growth |
| | | | | | | | | Factor Receptor Beta (PDGFR-Beta) |
| | | | | | | | | Antagonist; Proto-Oncogene |
| | | | | | | | | Tyrosine-Protein Kinase Receptor Ret |
| | | | | | | | | (RET Receptor Tyrosine Kinase or |
| | | | | | | | | Cadherin Family Member 12 or Proto- |
| | | | | | | | | Oncogene c-Ret or EC 2.7.10.1) |
| | | | | | | | | Inhibitor; Tie-2 Receptor Antagonist; |
| | | | | | | | | Vascular Endothelial Growth Factor |
| | | | | | | | | Receptor-1 (VEGFR-1) Antagonist; |
| | | | | | | | | Vascular Endothelial Growth Factor |
| | | | | | | | | Receptor-2 (VEGFR-2) Antagonist; |
| | | | | | | | | Vascular Endothelial Growth Factor |
| | | | | | | | | Receptor-3 (VEGFR-3) Antagonist |
| regorafenib | regorafenib | Stivarga | Bayer Australia Ltd. | Oncology | Gastrointestinal | Marketed | Australia | Abl Tyrosine Kinase Inhibitor; B-Raf |
| | | | | | Stromal Tumor | | | Kinase Inhibitor; c-kit Receptor |
| | | | | | (GIST); Metastatic | | | (SCFR, CD117) Antagonist; C-Raf |
| | | | | | Colorectal Cancer | | | Kinase Inhibitor; CD167b (Discoidin |
| | | | | | | | | Domain-Containing Receptor 2 or |
| | | | | | | | | Neurotrophic Tyrosine Kinase, |
| | | | | | | | | Receptor-Related 3) Inhibitor; |
| | | | | | | | | Fibroblast Growth Factor Receptor 1 |
| | | | | | | | | (FGFR1) Antagonist; Fibroblast |
| | | | | | | | | Growth Factor Receptor 2 |
| | | | | | | | | (Keratinocyte Growth Factor Receptor |
| | | | | | | | | or CD332 or FGFR2 or KGFR or K- |
| | | | | | | | | sam or EC 2.7.10.1) Antagonist; |
| | | | | | | | | Platelet Derived Growth Factor |
| | | | | | | | | Receptor Alpha (PDGFR-Alpha) |
| | | | | | | | | Antagonist; Platelet Derived Growth |
| | | | | | | | | Factor Receptor Beta (PDGFR-Beta) |
| | | | | | | | | Antagonist; Proto-Oncogene |
| | | | | | | | | Tyrosine-Protein Kinase Receptor Ret |
| | | | | | | | | (RET Receptor Tyrosine Kinase or |
| | | | | | | | | Cadherin Family Member 12 or Proto- |
| | | | | | | | | Oncogene c-Ret or EC 2.7.10.1) |
| | | | | | | | | Inhibitor; Tie-2 Receptor Antagonist; |
| | | | | | | | | Vascular Endothelial Growth Factor |
| | | | | | | | | Receptor-1 (VEGFR-1) Antagonist; |
| | | | | | | | | Vascular Endothelial Growth Factor |
| | | | | | | | | Receptor-2 (VEGFR-2) Antagonist; |
| | | | | | | | | Vascular Endothelial Growth Factor |
| | | | | | | | | Receptor-3 (VEGFR-3) Antagonist |
| regorafenib | regorafenib | Stivarga | Bayer Corporation | Oncology | Gastrointestinal | Marketed | United States | Abl Tyrosine Kinase Inhibitor; B-Raf |
| | | | | | Stromal Tumor | | | Kinase Inhibitor; c-kit Receptor |
| | | | | | (GIST); Metastatic | | | (SCFR, CD117) Antagonist; C-Raf |
| | | | | | Colorectal Cancer | | | Kinase Inhibitor; CD167b (Discoidin |
| | | | | | | | | Domain-Containing Receptor 2 or |
| | | | | | | | | Neurotrophic Tyrosine Kinase, |
| | | | | | | | | Receptor-Related 3) Inhibitor; |
| | | | | | | | | Fibroblast Growth Factor Receptor 1 |
| | | | | | | | | (FGFR1) Antagonist; Fibroblast |
| | | | | | | | | Growth Factor Receptor 2 |
| | | | | | | | | (Keratinocyte Growth Factor Receptor |
| | | | | | | | | or CD332 or FGFR2 or KGFR or K- |
| | | | | | | | | sam or EC 2.7.10.1) Antagonist; |
| | | | | | | | | Platelet Derived Growth Factor |
| | | | | | | | | Receptor Alpha (PDGFR-Alpha) |
| | | | | | | | | Antagonist; Platelet Derived Growth |
| | | | | | | | | Factor Receptor Beta (PDGFR-Beta) |
| | | | | | | | | Antagonist; Proto-Oncogene |
| | | | | | | | | Tyrosine-Protein Kinase Receptor Ret |
| | | | | | | | | (RET Receptor Tyrosine Kinase or |
| | | | | | | | | Cadherin Family Member 12 or Proto- |
| | | | | | | | | Oncogene c-Ret or EC 2.7.10.1) |
| | | | | | | | | Inhibitor; Tie-2 Receptor Antagonist; |
| | | | | | | | | Vascular Endothelial Growth Factor |
| | | | | | | | | Receptor-1 (VEGFR-1) Antagonist; |
| | | | | | | | | Vascular Endothelial Growth Factor |
| | | | | | | | | Receptor-2 (VEGFR-2) Antagonist; |
| | | | | | | | | Vascular Endothelial Growth Factor |
| | | | | | | | | Receptor-3 (VEGFR-3) Antagonist |
| regorafenib | regorafenib | Stivarga | Bayer HealthCare AG | Oncology | Gastrointestinal | Marketed | EU | Abl Tyrosine Kinase Inhibitor; B-Raf |
| | | | | | Stromal Tumor | | | Kinase Inhibitor; c-kit Receptor |
| | | | | | (GIST); Metastatic | | | (SCFR, CD117) Antagonist; C-Raf |
| | | | | | Colorectal Cancer | | | Kinase Inhibitor; CD167b (Discoidin |
| | | | | | | | | Domain-Containing Receptor 2 or |
| | | | | | | | | Neurotrophic Tyrosine Kinase, |
| | | | | | | | | Receptor-Related 3) Inhibitor; |
| | | | | | | | | Fibroblast Growth Factor Receptor 1 |
| | | | | | | | | (FGFR1) Antagonist; Fibroblast |
| | | | | | | | | Growth Factor Receptor 2 |
| | | | | | | | | (Keratinocyte Growth Factor Receptor |
| | | | | | | | | or CD332 or FGFR2 or KGFR or K- |
| | | | | | | | | sam or EC 2.7.10.1) Antagonist; |
| | | | | | | | | Platelet Derived Growth Factor |
| | | | | | | | | Receptor Alpha (PDGFR-Alpha) |
| | | | | | | | | Antagonist; Platelet Derived Growth |
| | | | | | | | | Factor Receptor Beta (PDGFR-Beta) |
| | | | | | | | | Antagonist; Proto-Oncogene |
| | | | | | | | | Tyrosine-Protein Kinase Receptor Ret |
| | | | | | | | | (RET Receptor Tyrosine Kinase or |
| | | | | | | | | Cadherin Family Member 12 or Proto- |
| | | | | | | | | Oncogene c-Ret or EC 2.7.10.1) |
| | | | | | | | | Inhibitor; Tie-2 Receptor Antagonist; |
| | | | | | | | | Vascular Endothelial Growth Factor |
| | | | | | | | | Receptor-1 (VEGFR-1) Antagonist; |
| | | | | | | | | Vascular Endothelial Growth Factor |
| | | | | | | | | Receptor-2 (VEGFR-2) Antagonist; |
| | | | | | | | | Vascular Endothelial Growth Factor |
| | | | | | | | | Receptor-3 (VEGFR-3) Antagonist |
| regorafenib | regorafenib | Stivarga | Bayer Hispania SL | Oncology | Gastrointestinal | Marketed | Spain | Abl Tyrosine Kinase Inhibitor; B-Raf |
| | | | | | Stromal Tumor | | | Kinase Inhibitor; c-kit Receptor |
| | | | | | (GIST); Metastatic | | | (SCFR, CD117) Antagonist; C-Raf |
| | | | | | Colorectal Cancer | | | Kinase Inhibitor; CD167b (Discoidin |
| | | | | | | | | Domain-Containing Receptor 2 or |
| | | | | | | | | Neurotrophic Tyrosine Kinase, |
| | | | | | | | | Receptor-Related 3) Inhibitor; |
| | | | | | | | | Fibroblast Growth Factor Receptor 1 |
| | | | | | | | | (FGFR1) Antagonist; Fibroblast |
| | | | | | | | | Growth Factor Receptor 2 |
| | | | | | | | | (Keratinocyte Growth Factor Receptor |
| | | | | | | | | or CD332 or FGFR2 or KGFR or K- |
| | | | | | | | | sam or EC 2.7.10.1) Antagonist; |
| | | | | | | | | Platelet Derived Growth Factor |
| | | | | | | | | Receptor Alpha (PDGFR-Alpha) |
| | | | | | | | | Antagonist; Platelet Derived Growth |
| | | | | | | | | Factor Receptor Beta (PDGFR-Beta) |
| | | | | | | | | Antagonist; Proto-Oncogene |
| | | | | | | | | Tyrosine-Protein Kinase Receptor Ret |
| | | | | | | | | (RET Receptor Tyrosine Kinase or |
| | | | | | | | | Cadherin Family Member 12 or Proto- |
| | | | | | | | | Oncogene c-Ret or EC 2.7.10.1) |
| | | | | | | | | Inhibitor; Tie-2 Receptor Antagonist; |
| | | | | | | | | Vascular Endothelial Growth Factor |
| | | | | | | | | Receptor-1 (VEGFR-1) Antagonist; |
| | | | | | | | | Vascular Endothelial Growth Factor |
| | | | | | | | | Receptor-2 (VEGFR-2) Antagonist; |
| | | | | | | | | Vascular Endothelial Growth Factor |
| | | | | | | | | Receptor-3 (VEGFR-3) Antagonist |
| regorafenib | regorafenib | Stivarga | Bayer Inc. | Oncology | Gastrointestinal | Marketed | Canada | Abl Tyrosine Kinase Inhibitor; B-Raf |
| | | | | | Stromal Tumor | | | Kinase Inhibitor; c-kit Receptor |
| | | | | | (GIST); Metastatic | | | (SCFR, CD117) Antagonist; C-Raf |
| | | | | | Colorectal Cancer | | | Kinase Inhibitor; CD167b (Discoidin |
| | | | | | | | | Domain-Containing Receptor 2 or |
| | | | | | | | | Neurotrophic Tyrosine Kinase, |
| | | | | | | | | Receptor-Related 3) Inhibitor; |
| | | | | | | | | Fibroblast Growth Factor Receptor 1 |
| | | | | | | | | (FGFR1) Antagonist; Fibroblast |
| | | | | | | | | Growth Factor Receptor 2 |
| | | | | | | | | (Keratinocyte Growth Factor Receptor |
| | | | | | | | | or CD332 or FGFR2 or KGFR or K- |
| | | | | | | | | sam or EC 2.7.10.1) Antagonist; |
| | | | | | | | | Platelet Derived Growth Factor |
| | | | | | | | | Receptor Alpha (PDGFR-Alpha) |
| | | | | | | | | Antagonist; Platelet Derived Growth |
| | | | | | | | | Factor Receptor Beta (PDGFR-Beta) |
| | | | | | | | | Antagonist; Proto-Oncogene |
| | | | | | | | | Tyrosine-Protein Kinase Receptor Ret |
| | | | | | | | | (RET Receptor Tyrosine Kinase or |
| | | | | | | | | Cadherin Family Member 12 or Proto- |
| | | | | | | | | Oncogene c-Ret or EC 2.7.10.1) |
| | | | | | | | | Inhibitor; Tie-2 Receptor Antagonist; |
| | | | | | | | | Vascular Endothelial Growth Factor |
| | | | | | | | | Receptor-1 (VEGFR-1) Antagonist; |
| | | | | | | | | Vascular Endothelial Growth Factor |
| | | | | | | | | Receptor-2 (VEGFR-2) Antagonist; |
| | | | | | | | | Vascular Endothelial Growth Factor |
| | | | | | | | | Receptor-3 (VEGFR-3) Antagonist |
| regorafenib | regorafenib | Stivarga | Bayer Korea Ltd. | Oncology | Gastrointestinal | Marketed | South Korea | Abl Tyrosine Kinase Inhibitor; B-Raf |
| | | | | | Stromal Tumor | | | Kinase Inhibitor; c-kit Receptor |
| | | | | | (GIST); Metastatic | | | (SCFR, CD117) Antagonist; C-Raf |
| | | | | | Colorectal Cancer | | | Kinase Inhibitor; CD167b (Discoidin |
| | | | | | | | | Domain-Containing Receptor 2 or |
| | | | | | | | | Neurotrophic Tyrosine Kinase, |
| | | | | | | | | Receptor-Related 3) Inhibitor; |
| | | | | | | | | Fibroblast Growth Factor Receptor 1 |
| | | | | | | | | (FGFR1) Antagonist; Fibroblast |
| | | | | | | | | Growth Factor Receptor 2 |
| | | | | | | | | (Keratinocyte Growth Factor Receptor |
| | | | | | | | | or CD332 or FGFR2 or KGFR or K- |
| | | | | | | | | sam or EC 2.7.10.1) Antagonist; |
| | | | | | | | | Platelet Derived Growth Factor |
| | | | | | | | | Receptor Alpha (PDGFR-Alpha) |
| | | | | | | | | Antagonist; Platelet Derived Growth |
| | | | | | | | | Factor Receptor Beta (PDGFR-Beta) |
| | | | | | | | | Antagonist; Proto-Oncogene |
| | | | | | | | | Tyrosine-Protein Kinase Receptor Ret |
| | | | | | | | | (RET Receptor Tyrosine Kinase or |
| | | | | | | | | Cadherin Family Member 12 or Proto- |
| | | | | | | | | Oncogene c-Ret or EC 2.7.10.1) |
| | | | | | | | | Inhibitor; Tie-2 Receptor Antagonist; |
| | | | | | | | | Vascular Endothelial Growth Factor |
| | | | | | | | | Receptor-1 (VEGFR-1) Antagonist; |
| | | | | | | | | Vascular Endothelial Growth Factor |
| | | | | | | | | Receptor-2 (VEGFR-2) Antagonist; |
| | | | | | | | | Vascular Endothelial Growth Factor |
| | | | | | | | | Receptor-3 (VEGFR-3) Antagonist |
| regorafenib | regorafenib | Stivarga | Bayer S.p.A | Oncology | Gastrointestinal | Marketed | Italy | Abl Tyrosine Kinase Inhibitor; B-Raf |
| | | | | | Stromal Tumor | | | Kinase Inhibitor; c-kit Receptor |
| | | | | | (GIST); Metastatic | | | (SCFR, CD117) Antagonist; C-Raf |
| | | | | | Colorectal Cancer | | | Kinase Inhibitor; CD167b (Discoidin |
| | | | | | | | | Domain-Containing Receptor 2 or |
| | | | | | | | | Neurotrophic Tyrosine Kinase, |
| | | | | | | | | Receptor-Related 3) Inhibitor; |
| | | | | | | | | Fibroblast Growth Factor Receptor 1 |
| | | | | | | | | (FGFR1) Antagonist; Fibroblast |
| | | | | | | | | Growth Factor Receptor 2 |
| | | | | | | | | (Keratinocyte Growth Factor Receptor |
| | | | | | | | | or CD332 or FGFR2 or KGFR or K- |
| | | | | | | | | sam or EC 2.7.10.1) Antagonist; |
| | | | | | | | | Platelet Derived Growth Factor |
| | | | | | | | | Receptor Alpha (PDGFR-Alpha) |
| | | | | | | | | Antagonist; Platelet Derived Growth |
| | | | | | | | | Factor Receptor Beta (PDGFR-Beta) |
| | | | | | | | | Antagonist; Proto-Oncogene |
| | | | | | | | | Tyrosine-Protein Kinase Receptor Ret |
| | | | | | | | | (RET Receptor Tyrosine Kinase or |
| | | | | | | | | Cadherin Family Member 12 or Proto- |
| | | | | | | | | Oncogene c-Ret or EC 2.7.10.1) |
| | | | | | | | | Inhibitor; Tie-2 Receptor Antagonist; |
| | | | | | | | | Vascular Endothelial Growth Factor |
| | | | | | | | | Receptor-1 (VEGFR-1) Antagonist; |
| | | | | | | | | Vascular Endothelial Growth Factor |
| | | | | | | | | Receptor-2 (VEGFR-2) Antagonist; |
| | | | | | | | | Vascular Endothelial Growth Factor |
| | | | | | | | | Receptor-3 (VEGFR-3) Antagonist |
| regorafenib | regorafenib | Stivarga | Bayer Sante SAS | Oncology | Gastrointestinal | Marketed | France | Abl Tyrosine Kinase Inhibitor; B-Raf |
| | | | | | Stromal Tumor | | | Kinase Inhibitor; c-kit Receptor |
| | | | | | (GIST); Metastatic | | | (SCFR, CD117) Antagonist; C-Raf |
| | | | | | Colorectal Cancer | | | Kinase Inhibitor; CD167b (Discoidin |
| | | | | | | | | Domain-Containing Receptor 2 or |
| | | | | | | | | Neurotrophic Tyrosine Kinase, |
| | | | | | | | | Receptor-Related 3) Inhibitor; |
| | | | | | | | | Fibroblast Growth Factor Receptor 1 |
| | | | | | | | | (FGFR1) Antagonist; Fibroblast |
| | | | | | | | | Growth Factor Receptor 2 |
| | | | | | | | | (Keratinocyte Growth Factor Receptor |
| | | | | | | | | or CD332 or FGFR2 or KGFR or K- |
| | | | | | | | | sam or EC 2.7.10.1) Antagonist; |
| | | | | | | | | Platelet Derived Growth Factor |
| | | | | | | | | Receptor Alpha (PDGFR-Alpha) |
| | | | | | | | | Antagonist; Platelet Derived Growth |
| | | | | | | | | Factor Receptor Beta (PDGFR-Beta) |
| | | | | | | | | Antagonist; Proto-Oncogene |
| | | | | | | | | Tyrosine-Protein Kinase Receptor Ret |
| | | | | | | | | (RET Receptor Tyrosine Kinase or |
| | | | | | | | | Cadherin Family Member 12 or Proto- |
| | | | | | | | | Oncogene c-Ret or EC 2.7.10.1) |
| | | | | | | | | Inhibitor; Tie-2 Receptor Antagonist; |
| | | | | | | | | Vascular Endothelial Growth Factor |
| | | | | | | | | Receptor-1 (VEGFR-1) Antagonist; |
| | | | | | | | | Vascular Endothelial Growth Factor |
| | | | | | | | | Receptor-2 (VEGFR-2) Antagonist; |
| | | | | | | | | Vascular Endothelial Growth Factor |
| | | | | | | | | Receptor-3 (VEGFR-3) Antagonist |
| regorafenib | regorafenib | Stivarga | Bayer UK Limited | Oncology | Gastrointestinal | Marketed | United Kingdom | Abl Tyrosine Kinase Inhibitor; B-Raf |
| | | | | | Stromal Tumor | | | Kinase Inhibitor; c-kit Receptor |
| | | | | | (GIST); Metastatic | | | (SCFR, CD117) Antagonist; C-Raf |
| | | | | | Colorectal Cancer | | | Kinase Inhibitor; CD167b (Discoidin |
| | | | | | | | | Domain-Containing Receptor 2 or |
| | | | | | | | | Neurotrophic Tyrosine Kinase, |
| | | | | | | | | Receptor-Related 3) Inhibitor; |
| | | | | | | | | Fibroblast Growth Factor Receptor 1 |
| | | | | | | | | (FGFR1) Antagonist; Fibroblast |
| | | | | | | | | Growth Factor Receptor 2 |
| | | | | | | | | (Keratinocyte Growth Factor Receptor |
| | | | | | | | | or CD332 or FGFR2 or KGFR or K- |
| | | | | | | | | sam or EC 2.7.10.1) Antagonist; |
| | | | | | | | | Platelet Derived Growth Factor |
| | | | | | | | | Receptor Alpha (PDGFR-Alpha) |
| | | | | | | | | Antagonist; Platelet Derived Growth |
| | | | | | | | | Factor Receptor Beta (PDGFR-Beta) |
| | | | | | | | | Antagonist; Proto-Oncogene |
| | | | | | | | | Tyrosine-Protein Kinase Receptor Ret |
| | | | | | | | | (RET Receptor Tyrosine Kinase or |
| | | | | | | | | Cadherin Family Member 12 or Proto- |
| | | | | | | | | Oncogene c-Ret or EC 2.7.10.1) |
| | | | | | | | | Inhibitor; Tie-2 Receptor Antagonist; |
| | | | | | | | | Vascular Endothelial Growth Factor |
| | | | | | | | | Receptor-1 (VEGFR-1) Antagonist; |
| | | | | | | | | Vascular Endothelial Growth Factor |
| | | | | | | | | Receptor-2 (VEGFR-2) Antagonist; |
| | | | | | | | | Vascular Endothelial Growth Factor |
| | | | | | | | | Receptor-3 (VEGFR-3) Antagonist |
| regorafenib | regorafenib | Stivarga | Bayer Yakuhin, Ltd. | Oncology | Gastrointestinal | Marketed | Japan | Abl Tyrosine Kinase Inhibitor; B-Raf |
| | | | | | Stromal Tumor | | | Kinase Inhibitor; c-kit Receptor |
| | | | | | (GIST); Metastatic | | | (SCFR, CD117) Antagonist; C-Raf |
| | | | | | Colorectal Cancer | | | Kinase Inhibitor; CD167b (Discoidin |
| | | | | | | | | Domain-Containing Receptor 2 or |
| | | | | | | | | Neurotrophic Tyrosine Kinase, |
| | | | | | | | | Receptor-Related 3) Inhibitor; |
| | | | | | | | | Fibroblast Growth Factor Receptor 1 |
| | | | | | | | | (FGFR1) Antagonist; Fibroblast |
| | | | | | | | | Growth Factor Receptor 2 |
| | | | | | | | | (Keratinocyte Growth Factor Receptor |
| | | | | | | | | or CD332 or FGFR2 or KGFR or K- |
| | | | | | | | | sam or EC 2.7.10.1) Antagonist; |
| | | | | | | | | Platelet Derived Growth Factor |
| | | | | | | | | Receptor Alpha (PDGFR-Alpha) |
| | | | | | | | | Antagonist; Platelet Derived Growth |
| | | | | | | | | Factor Receptor Beta (PDGFR-Beta) |
| | | | | | | | | Antagonist; Proto-Oncogene |
| | | | | | | | | Tyrosine-Protein Kinase Receptor Ret |
| | | | | | | | | (RET Receptor Tyrosine Kinase or |
| | | | | | | | | Cadherin Family Member 12 or Proto- |
| | | | | | | | | Oncogene c-Ret or EC 2.7.10.1) |
| | | | | | | | | Inhibitor; Tie-2 Receptor Antagonist; |
| | | | | | | | | Vascular Endothelial Growth Factor |
| | | | | | | | | Receptor-1 (VEGFR-1) Antagonist; |
| | | | | | | | | Vascular Endothelial Growth Factor |
| | | | | | | | | Receptor-2 (VEGFR-2) Antagonist; |
| | | | | | | | | Vascular Endothelial Growth Factor |
| | | | | | | | | Receptor-3 (VEGFR-3) Antagonist |
| regorafenib | regorafenib | Stivarga | Bayern | Oncology | Gastrointestinal | Marketed | Germany | Abl Tyrosine Kinase Inhibitor; B-Raf |
| | | | International | | Stromal Tumor | | | Kinase Inhibitor; c-kit Receptor |
| | | | GmbH | | (GIST); Metastatic | | | (SCFR, CD117) Antagonist; C-Raf |
| | | | | | Colorectal Cancer | | | Kinase Inhibitor; CD167b (Discoidin |
| | | | | | | | | Domain-Containing Receptor 2 or |
| | | | | | | | | Neurotrophic Tyrosine Kinase, |
| | | | | | | | | Receptor-Related 3) Inhibitor; |
| | | | | | | | | Fibroblast Growth Factor Receptor 1 |
| | | | | | | | | (FGFR1) Antagonist; Fibroblast |
| | | | | | | | | Growth Factor Receptor 2 |
| | | | | | | | | (Keratinocyte Growth Factor Receptor |
| | | | | | | | | or CD332 or FGFR2 or KGFR or K- |
| | | | | | | | | sam or EC 2.7.10.1) Antagonist; |
| | | | | | | | | Platelet Derived Growth Factor |
| | | | | | | | | Receptor Alpha (PDGFR-Alpha) |
| | | | | | | | | Antagonist; Platelet Derived Growth |
| | | | | | | | | Factor Receptor Beta (PDGFR-Beta) |
| | | | | | | | | Antagonist; Proto-Oncogene |
| | | | | | | | | Tyrosine-Protein Kinase Receptor Ret |
| | | | | | | | | (RET Receptor Tyrosine Kinase or |
| | | | | | | | | Cadherin Family Member 12 or Proto- |
| | | | | | | | | Oncogene c-Ret or EC 2.7.10.1) |
| | | | | | | | | Inhibitor; Tie-2 Receptor Antagonist; |
| | | | | | | | | Vascular Endothelial Growth Factor |
| | | | | | | | | Receptor-1 (VEGFR-1) Antagonist; |
| | | | | | | | | Vascular Endothelial Growth Factor |
| | | | | | | | | Receptor-2 (VEGFR-2) Antagonist; |
| | | | | | | | | Vascular Endothelial Growth Factor |
| | | | | | | | | Receptor-3 (VEGFR-3) Antagonist |
| regorafenib | regorafenib | | Bayer AG | Oncology | Hepatocellular | Phase III | Global | Abl Tyrosine Kinase Inhibitor; B-Raf |
| | | | | | Carcinoma | | | Kinase Inhibitor; c-kit Receptor |
| | | | | | | | | (SCFR, CD117) Antagonist; C-Raf |
| | | | | | | | | Kinase Inhibitor; CD167b (Discoidin |
| | | | | | | | | Domain-Containing Receptor 2 or |
| | | | | | | | | Neurotrophic Tyrosine Kinase, |
| | | | | | | | | Receptor-Related 3) Inhibitor; |
| | | | | | | | | Fibroblast Growth Factor Receptor 1 |
| | | | | | | | | (FGFR1) Antagonist; Fibroblast |
| | | | | | | | | Growth Factor Receptor 2 |
| | | | | | | | | (Keratinocyte Growth Factor Receptor |
| | | | | | | | | or CD332 or FGFR2 or KGFR or K- |
| | | | | | | | | sam or EC 2.7.10.1) Antagonist; |
| | | | | | | | | Platelet Derived Growth Factor |
| | | | | | | | | Receptor Alpha (PDGFR-Alpha) |
| | | | | | | | | Antagonist; Platelet Derived Growth |
| | | | | | | | | Factor Receptor Beta (PDGFR-Beta) |
| | | | | | | | | Antagonist; Proto-Oncogene |
| | | | | | | | | Tyrosine-Protein Kinase Receptor Ret |
| | | | | | | | | (RET Receptor Tyrosine Kinase or |
| | | | | | | | | Cadherin Family Member 12 or Proto- |
| | | | | | | | | Oncogene c-Ret or EC 2.7.10.1) |
| | | | | | | | | Inhibitor; Tie-2 Receptor Antagonist; |
| | | | | | | | | Vascular Endothelial Growth Factor |
| | | | | | | | | Receptor-1 (VEGFR-1) Antagonist; |
| | | | | | | | | Vascular Endothelial Growth Factor |
| | | | | | | | | Receptor-2 (VEGFR-2) Antagonist; |
| | | | | | | | | Vascular Endothelial Growth Factor |
| | | | | | | | | Receptor-3 (VEGFR-3) Antagonist |
| regorafenib | regorafenib | | Bayer AG | Oncology | Metastatic | Phase III | Asia- | Abl Tyrosine Kinase Inhibitor; B-Raf |
| | | | | | Colorectal Cancer | | Pacific; | Kinase Inhibitor; c-kit Receptor |
| | | | | | | | Global | (SCFR, CD117) Antagonist; C-Raf |
| | | | | | | | | Kinase Inhibitor; CD167b (Discoidin |
| | | | | | | | | Domain-Containing Receptor 2 or |
| | | | | | | | | Neurotrophic Tyrosine Kinase, |
| | | | | | | | | Receptor-Related 3) Inhibitor; |
| | | | | | | | | Fibroblast Growth Factor Receptor 1 |
| | | | | | | | | (FGFR1) Antagonist; Fibroblast |
| | | | | | | | | Growth Factor Receptor 2 |
| | | | | | | | | (Keratinocyte Growth Factor Receptor |
| | | | | | | | | or CD332 or FGFR2 or KGFR or K- |
| | | | | | | | | sam or EC 2.7.10.1) Antagonist; |
| | | | | | | | | Platelet Derived Growth Factor |
| | | | | | | | | Receptor Alpha (PDGFR-Alpha) |
| | | | | | | | | Antagonist; Platelet Derived Growth |
| | | | | | | | | Factor Receptor Beta (PDGFR-Beta) |
| | | | | | | | | Antagonist; Proto-Oncogene |
| | | | | | | | | Tyrosine-Protein Kinase Receptor Ret |
| | | | | | | | | (RET Receptor Tyrosine Kinase or |
| | | | | | | | | Cadherin Family Member 12 or Proto- |
| | | | | | | | | Oncogene c-Ret or EC 2.7.10.1) |
| | | | | | | | | Inhibitor; Tie-2 Receptor Antagonist; |
| | | | | | | | | Vascular Endothelial Growth Factor |
| | | | | | | | | Receptor-1 (VEGFR-1) Antagonist; |
| | | | | | | | | Vascular Endothelial Growth Factor |
| | | | | | | | | Receptor-2 (VEGFR-2) Antagonist; |
| | | | | | | | | Vascular Endothelial Growth Factor |
| | | | | | | | | Receptor-3 (VEGFR-3) Antagonist |
| regorafenib | regorafenib | | Bayer AG | Oncology | Bile Duct Cancer | Phase II | Global | Abl Tyrosine Kinase Inhibitor; B-Raf |
| | | | | | (Cholangiocarcinoma); | | | Kinase Inhibitor; c-kit Receptor |
| | | | | | Epithelial | | | (SCFR, CD117) Antagonist; C-Raf |
| | | | | | Ovarian Cancer; | | | Kinase Inhibitor; CD167b (Discoidin |
| | | | | | Fallopian Tube | | | Domain-Containing Receptor 2 or |
| | | | | | Cancer; Metastatic | | | Neurotrophic Tyrosine Kinase, |
| | | | | | Colorectal Cancer; | | | Receptor-Related 3) Inhibitor; |
| | | | | | Pancreatic Cancer; | | | Fibroblast Growth Factor Receptor 1 |
| | | | | | Peritoneal Cancer; | | | (FGFR1) Antagonist; Fibroblast |
| | | | | | Renal Cell | | | Growth Factor Receptor 2 |
| | | | | | Carcinoma; | | | (Keratinocyte Growth Factor Receptor |
| | | | | | Salivary Gland | | | or CD332 or FGFR2 or KGFR or K- |
| | | | | | Cancer; Soft | | | sam or EC 2.7.10.1) Antagonist; |
| | | | | | Tissue Sarcoma; | | | Platelet Derived Growth Factor |
| | | | | | Transitional | | | Receptor Alpha (PDGFR-Alpha) |
| | | | | | Cell Cancer | | | Antagonist; Platelet Derived Growth |
| | | | | | (Urothelial | | | Factor Receptor Beta (PDGFR-Beta) |
| | | | | | Cell Cancer) | | | Antagonist; Proto-Oncogene |
| | | | | | | | | Tyrosine-Protein Kinase Receptor Ret |
| | | | | | | | | (RET Receptor Tyrosine Kinase or |
| | | | | | | | | Cadherin Family Member 12 or Proto- |
| | | | | | | | | Oncogene c-Ret or EC 2.7.10.1) |
| | | | | | | | | Inhibitor; Tie-2 Receptor Antagonist; |
| | | | | | | | | Vascular Endothelial Growth Factor |
| | | | | | | | | Receptor-1 (VEGFR-1) Antagonist; |
| | | | | | | | | Vascular Endothelial Growth Factor |
| | | | | | | | | Receptor-2 (VEGFR-2) Antagonist; |
| | | | | | | | | Vascular Endothelial Growth Factor |
| | | | | | | | | Receptor-3 (VEGFR-3) Antagonist |
| regorafenib | regorafenib | | Bayer AG | Oncology | Non-Small | Inactive | Global | Abl Tyrosine Kinase Inhibitor; B-Raf |
| | | | | | Cell Lung Cancer | | | Kinase Inhibitor; c-kit Receptor |
| | | | | | | | | (SCFR, CD117) Antagonist; C-Raf |
| | | | | | | | | Kinase Inhibitor; CD167b (Discoidin |
| | | | | | | | | Domain-Containing Receptor 2 or |
| | | | | | | | | Neurotrophic Tyrosine Kinase, |
| | | | | | | | | Receptor-Related 3) Inhibitor; |
| | | | | | | | | Fibroblast Growth Factor Receptor 1 |
| | | | | | | | | (FGFR1) Antagonist; Fibroblast |
| | | | | | | | | Growth Factor Receptor 2 |
| | | | | | | | | (Keratinocyte Growth Factor Receptor |
| | | | | | | | | or CD332 or FGFR2 or KGFR or K- |
| | | | | | | | | sam or EC 2.7.10.1) Antagonist; |
| | | | | | | | | Platelet Derived Growth Factor |
| | | | | | | | | Receptor Alpha (PDGFR-Alpha) |
| | | | | | | | | Antagonist; Platelet Derived Growth |
| | | | | | | | | Factor Receptor Beta (PDGFR-Beta) |
| | | | | | | | | Antagonist; Proto-Oncogene |
| | | | | | | | | Tyrosine-Protein Kinase Receptor Ret |
| | | | | | | | | (RET Receptor Tyrosine Kinase or |
| | | | | | | | | Cadherin Family Member 12 or Proto- |
| | | | | | | | | Oncogene c-Ret or EC 2.7.10.1) |
| | | | | | | | | Inhibitor; Tie-2 Receptor Antagonist; |
| | | | | | | | | Vascular Endothelial Growth Factor |
| | | | | | | | | Receptor-1 (VEGFR-1) Antagonist; |
| | | | | | | | | Vascular Endothelial Growth Factor |
| | | | | | | | | Receptor-2 (VEGFR-2) Antagonist; |
| | | | | | | | | Vascular Endothelial Growth Factor |
| | | | | | | | | Receptor-3 (VEGFR-3) Antagonist |
| regorafenib | regorafenib | | Bayer AG | Oncology | Solid Tumor | Inactive | China; Japan | Abl Tyrosine Kinase Inhibitor; B-Raf |
| | | | | | | | | Kinase Inhibitor; c-kit Receptor |
| | | | | | | | | (SCFR, CD117) Antagonist; C-Raf |
| | | | | | | | | Kinase Inhibitor; CD167b (Discoidin |
| | | | | | | | | Domain-Containing Receptor 2 or |
| | | | | | | | | Neurotrophic Tyrosine Kinase, |
| | | | | | | | | Receptor-Related 3) Inhibitor; |
| | | | | | | | | Fibroblast Growth Factor Receptor 1 |
| | | | | | | | | (FGFR1) Antagonist; Fibroblast |
| | | | | | | | | Growth Factor Receptor 2 |
| | | | | | | | | (Keratinocyte Growth Factor Receptor |
| | | | | | | | | or CD332 or FGFR2 or KGFR or K- |
| | | | | | | | | sam or EC 2.7.10.1) Antagonist; |
| | | | | | | | | Platelet Derived Growth Factor |
| | | | | | | | | Receptor Alpha (PDGFR-Alpha) |
| | | | | | | | | Antagonist; Platelet Derived Growth |
| | | | | | | | | Factor Receptor Beta (PDGFR-Beta) |
| | | | | | | | | Antagonist; Proto-Oncogene |
| | | | | | | | | Tyrosine-Protein Kinase Receptor Ret |
| | | | | | | | | (RET Receptor Tyrosine Kinase or |
| | | | | | | | | Cadherin Family Member 12 or Proto- |
| | | | | | | | | Oncogene c-Ret or EC 2.7.10.1) |
| | | | | | | | | Inhibitor; Tie-2 Receptor Antagonist; |
| | | | | | | | | Vascular Endothelial Growth Factor |
| | | | | | | | | Receptor-1 (VEGFR-1) Antagonist; |
| | | | | | | | | Vascular Endothelial Growth Factor |
| | | | | | | | | Receptor-2 (VEGFR-2) Antagonist; |
| | | | | | | | | Vascular Endothelial Growth Factor |
| | | | | | | | | Receptor-3 (VEGFR-3) Antagonist |
| regorafenib | regorafenib | | Bayer AG | Ophthalmology | Wet | Discontinued | Global | Abl Tyrosine Kinase Inhibitor; B-Raf |
| | | | | | (Neovascular/ | | | Kinase Inhibitor; c-kit Receptor |
| | | | | | Exudative) Macular | | | (SCFR, CD117) Antagonist; C-Raf |
| | | | | | Degeneration | | | Kinase Inhibitor; CD167b (Discoidin |
| | | | | | | | | Domain-Containing Receptor 2 or |
| | | | | | | | | Neurotrophic Tyrosine Kinase, |
| | | | | | | | | Receptor-Related 3) Inhibitor; |
| | | | | | | | | Fibroblast Growth Factor Receptor 1 |
| | | | | | | | | (FGFR1) Antagonist; Fibroblast |
| | | | | | | | | Growth Factor Receptor 2 |
| | | | | | | | | (Keratinocyte Growth Factor Receptor |
| | | | | | | | | or CD332 or FGFR2 or KGFR or K- |
| | | | | | | | | sam or EC 2.7.10.1) Antagonist; |
| | | | | | | | | Platelet Derived Growth Factor |
| | | | | | | | | Receptor Alpha (PDGFR-Alpha) |
| | | | | | | | | Antagonist; Platelet Derived Growth |
| | | | | | | | | Factor Receptor Beta (PDGFR-Beta) |
| | | | | | | | | Antagonist; Proto-Oncogene |
| | | | | | | | | Tyrosine-Protein Kinase Receptor Ret |
| | | | | | | | | (RET Receptor Tyrosine Kinase or |
| | | | | | | | | Cadherin Family Member 12 or Proto- |
| | | | | | | | | Oncogene c-Ret or EC 2.7.10.1) |
| | | | | | | | | Inhibitor; Tie-2 Receptor Antagonist; |
| | | | | | | | | Vascular Endothelial Growth Factor |
| | | | | | | | | Receptor-1 (VEGFR-1) Antagonist; |
| | | | | | | | | Vascular Endothelial Growth Factor |
| | | | | | | | | Receptor-2 (VEGFR-2) Antagonist; |
| | | | | | | | | Vascular Endothelial Growth Factor |
| | | | | | | | | Receptor-3 (VEGFR-3) Antagonist |
| dabrafenib | dabrafenib | Tafinlar | Novartis | Oncology | Metastatic Melanoma | Marketed | United States | B-Raf Kinase Inhibitor |
| mesylate | mesylate | | Corporation | | | | | |
| dabrafenib | dabrafenib | Tafinlar | Novartis | Oncology | Metastatic Melanoma | Marketed | United Kingdom | B-Raf Kinase Inhibitor |
| mesylate | mesylate | | Europharm Ltd. | | | | | |
| dabrafenib | dabrafenib | Tafinlar | Novartis | Oncology | Metastatic Melanoma | Marketed | Italy | B-Raf Kinase Inhibitor |
| mesylate | mesylate | | Farma S.p.A | | | | | |
| dabrafenib | dabrafenib | Tafinlar | Novartis | Oncology | Metastatic Melanoma | Marketed | Spain | B-Raf Kinase Inhibitor |
| mesylate | mesylate | | Farmaceutica, SA | | | | | |
| dabrafenib | dabrafenib | Tafinlar | Novartis | Oncology | Metastatic Melanoma | Marketed | Germany | B-Raf Kinase Inhibitor |
| mesylate | mesylate | | Pharma GmbH | | | | | |
| dabrafenib | dabrafenib | Tafinlar | Novartis | Oncology | Metastatic Melanoma | Marketed | France | B-Raf Kinase Inhibitor |
| mesylate | mesylate | | Pharma S.A.S. | | | | | |
| dabrafenib | dabrafenib | Tafinlar | Novartis | Oncology | Metastatic Melanoma | Marketed | Australia | B-Raf Kinase Inhibitor |
| mesylate | mesylate | | Pharmaceuticals | | | | | |
| | | | Australia | | | | | |
| | | | Pty Limited | | | | | |
| dabrafenib | dabrafenib | Tafinlar | Novartis | Oncology | Metastatic Melanoma | Marketed | Canada | B-Raf Kinase Inhibitor |
| mesylate | mesylate | | Pharmaceuticals | | | | | |
| | | | Canada Inc. | | | | | |
| dabrafenib | dabrafenib | Tafinlar | Novartis | Oncology | Metastatic Melanoma | Marketed | EU | B-Raf Kinase Inhibitor |
| mesylate | mesylate | | Pharmaceuticals | | | | | |
| | | | UK Limited | | | | | |
| dabrafenib | dabrafenib | | Novartis AG | Oncology | Metastatic Melanoma | Phase III | Global | B-Raf Kinase Inhibitor |
| mesylate | mesylate | | | | | | | |
| dabrafenib | dabrafenib | | Novartis AG | Oncology | Follicular | Phase II | Global | B-Raf Kinase Inhibitor |
| mesylate | mesylate | | | | Thyroid Cancer; | | | |
| | | | | | Papillary | | | |
| | | | | | Thyroid Cancer | | | |
| dabrafenib | dabrafenib | Tafinlar | Novartis AG | Oncology | Metastatic | Phase II | Global | B-Raf Kinase Inhibitor |
| mesylate | mesylate | | | | Colorectal Cancer; | | | |
| | | | | | Non-Small | | | |
| | | | | | Cell Lung Cancer | | | |
| dabrafenib | dabrafenib | Tafinlar + | Novartis | Oncology | Metastatic Melanoma | Marketed | United States | B-Raf Kinase Inhibitor; Mitogen |
| mesylate + | mesylate + | Mekinist | Corporation | | | | | Activated Protein Kinase Kinase 1 |
| trametinib | trametinib | | | | | | | (MEK-1 or MAP2K1) Inhibitor; |
| dimethyl | dimethyl | | | | | | | Mitogen Activated Protein Kinase |
| sulfoxide | sulfoxide | | | | | | | Kinase 2 (MEK-2 or MAP2K2) |
| | | | | | | | | Inhibitor |
| dabrafenib | dabrafenib | Tafinlar + | Novartis | Oncology | Metastatic Melanoma | Marketed | EU | B-Raf Kinase Inhibitor; Mitogen |
| mesylate + | mesylate + | Mekinist | Europharm Ltd. | | | | | Activated Protein Kinase Kinase 1 |
| trametinib | trametinib | | | | | | | (MEK-1 or MAP2K1) Inhibitor; |
| dimethyl | dimethyl | | | | | | | Mitogen Activated Protein Kinase |
| sulfoxide | sulfoxide | | | | | | | Kinase 2 (MEK-2 or MAP2K2) |
| | | | | | | | | Inhibitor |
| dabrafenib | dabrafenib | Sulfoxide | Novartis AG | Oncology | Metastatic Melanoma | Pre- | Japan | B-Raf Kinase Inhibitor; Mitogen |
| mesylate + | mesylate + | | | | | Registration | | Activated Protein Kinase Kinase 1 |
| trametinib | trametinib | | | | | | | (MEK-1 or MAP2K1) Inhibitor; |
| dimethyl | dimethyl | | | | | | | Mitogen Activated Protein Kinase |
| sulfoxide | sulfoxide | | | | | | | Kinase 2 (MEK-2 or MAP2K2) |
| | | | | | | | | Inhibitor |
| dabrafenib | dabrafenib | Sulfoxide | Novartis AG | Oncology | Melanoma; | Phase III | Global | B-Raf Kinase Inhibitor; Mitogen |
| mesylate + | mesylate + | | | | Metastatic Melanoma | | | Activated Protein Kinase Kinase 1 |
| trametinib | trametinib | | | | | | | (MEK-1 or MAP2K1) Inhibitor; |
| dimethyl | dimethyl | | | | | | | Mitogen Activated Protein Kinase |
| sulfoxide | sulfoxide | | | | | | | Kinase 2 (MEK-2 or MAP2K2) |
| | | | | | | | | Inhibitor |
| dabrafenib | dabrafenib | Sulfoxide | Novartis AG | Oncology | Acral Lentiginous | Phase II | Global | B-Raf Kinase Inhibitor; Mitogen |
| mesylate + | mesylate + | | | | Melanoma; | | | Activated Protein Kinase Kinase 1 |
| trametinib | trametinib | | | | Adenocarcinoma; | | | (MEK-1 or MAP2K1) Inhibitor; |
| dimethyl | dimethyl | | | | Anaplastic | | | Mitogen Activated Protein Kinase |
| sulfoxide | sulfoxide | | | | Thyroid Cancer; | | | Kinase 2 (MEK-2 or MAP2K2) |
| | | | | | Biliary Tumor; | | | Inhibitor |
| | | | | | Colorectal Cancer; | | | |
| | | | | | Gastrointestinal | | | |
| | | | | | Stromal Tumor | | | |
| | | | | | (GIST); Glioma; | | | |
| | | | | | Hairy Cell | | | |
| | | | | | Leukemia; | | | |
| | | | | | High-Grade | | | |
| | | | | | Glioma; Metastatic | | | |
| | | | | | Brain Tumor; | | | |
| | | | | | Multiple Myeloma | | | |
| | | | | | (Kahler Disease); | | | |
| | | | | | Non-Small | | | |
| | | | | | Cell Lung Cancer; | | | |
| | | | | | Nongerminomatous | | | |
| | | | | | (Nonseminomatous) | | | |
| | | | | | Germ Cell Tumors | | | |
| dabrafenib | dabrafenib | Sulfoxide | Novartis AG | Oncology | Solid Tumor | Phase II | Japan | B-Raf Kinase Inhibitor; Mitogen |
| mesylate + | mesylate + | | | | | | | Activated Protein Kinase Kinase 1 |
| trametinib | trametinib | | | | | | | (MEK-1 or MAP2K1) Inhibitor; |
| dimethyl | dimethyl | | | | | | | Mitogen Activated Protein Kinase |
| sulfoxide | sulfoxide | | | | | | | Kinase 2 (MEK-2 or MAP2K2) |
| | | | | | | | | Inhibitor |
| sorafenib | sorafenib | Sorafenib | Cipla Ltd. | Oncology | Renal Cell | Marketed | India | B-Raf Kinase Inhibitor; c-kit Receptor |
| tosylate | tosylate | Tosylate | | | Carcinoma | | | (SCFR, CD117) Antagonist; C-Raf |
| | | | | | | | | Kinase Inhibitor; Platelet Derived |
| | | | | | | | | Growth Factor Receptor Beta |
| | | | | | | | | (PDGFR-Beta) Antagonist; Receptor- |
| | | | | | | | | Type Tyrosine-Protein Kinase FLT3 |
| | | | | | | | | (FMS-Like Tyrosine Kinase 3 or FL |
| | | | | | | | | Cytokine Receptor or Stem Cell |
| | | | | | | | | Tyrosine Kinase 1 or CD135 or Fetal |
| | | | | | | | | Liver Kinase-2 or EC 2.7.10.1) |
| | | | | | | | | Antagonist; Vascular Endothelial |
| | | | | | | | | Growth Factor Receptor-2 (VEGFR-2) |
| | | | | | | | | Antagonist; Vascular Endothelial |
| | | | | | | | | Growth Factor Receptor-3 (VEGFR-3) |
| | | | | | | | | Antagonist |
| binimetinib + | binimetinib + | | Array | Oncology | Metastatic Melanoma | Phase III | Global | B-Raf Kinase Inhibitor; Mitogen |
| encorafenib | encorafenib | | BioPharma Inc. | | | | | Activated Protein Kinase Kinase 1 |
| | | | | | | | | (MEK-1 or MAP2K1) Inhibitor; |
| | | | | | | | | Mitogen Activated Protein Kinase |
| | | | | | | | | Kinase 2 (MEK-2 or MAP2K2) |
| | | | | | | | | Inhibitor |
| binimetinib + | binimetinib + | | Array | Oncology | Metastatic | Phase II | Global | B-Raf Kinase Inhibitor; Mitogen |
| encorafenib | encorafenib | | BioPharma Inc. | | Colorectal Cancer; | | | Activated Protein Kinase Kinase 1 |
| | | | | | Solid Tumor | | | (MEK-1 or MAP2K1) Inhibitor; |
| | | | | | | | | Mitogen Activated Protein Kinase |
| | | | | | | | | Kinase 2 (MEK-2 or MAP2K2) |
| | | | | | | | | Inhibitor |
| encorafenib | encorafenib | | Array | Oncology | Metastatic Melanoma | Phase III | Global | B-Raf Kinase Inhibitor |
| | | | BioPharma Inc. | | | | | |
| encorafenib | encorafenib | | Array | Oncology | Hematological | Phase II | Global | B-Raf Kinase Inhibitor |
| | | | BioPharma Inc. | | Tumor; Metastatic | | | |
| | | | | | Colorectal Cancer; | | | |
| | | | | | Solid Tumor | | | |
| encorafenib | encorafenib | | Array | Oncology | Thyroid Cancer | Unknown | Global | B-Raf Kinase Inhibitor |
| | | | BioPharma Inc. | | | | | |
| encorafenib | encorafenib | | Array | Oncology | Non-Small | Inactive | Global | B-Raf Kinase Inhibitor |
| | | | BioPharma Inc. | | Cell Lung Cancer | | | |
| dabrafenib | dabrafenib | | GlaxoSmithKline Plc | Oncology | Colorectal Cancer | Phase II | Global | B-Raf Kinase Inhibitor; Epidermal |
| mesylate + | mesylate + | | | | | | | Growth Factor Receptor (EGFR, |
| panitumumab + | panitumumab + | | | | | | | HER-1 or ErbB-1) Antagonist; |
| trametinib | trametinib | | | | | | | Mitogen Activated Protein Kinase |
| dimethyl | dimethyl | | | | | | | Kinase 1 (MEK-1 or MAP2K1) |
| sulfoxide | sulfoxide | | | | | | | Inhibitor; Mitogen Activated Protein |
| | | | | | | | | Kinase Kinase 2 (MEK-2 or MAP2K2) |
| | | | | | | | | Inhibitor |
| donafenib | | | Suzhou Zelgen | Oncology | Esophageal Cancer; | Phase II | Global | Raf Kinase Inhibitor; Receptor |
| | | | Biopharmaceutical | | Gastric Cancer; | | | Tyrosine Kinase Inhibitor |
| | | | Co., Ltd. | | Hepatocellular | | | |
| | | | | | Carcinoma; Metastatic | | | |
| | | | | | Colorectal Cancer | | | |
| NEO-100 | | | Neonc | Oncology | Recurrent | Phase II | Global | G Protein-Coupled Receptor 78 |
| | | | Technologies, Inc. | | Glioblastoma | | | (GPR78) Agonist; Ras Inhibitor |
| | | | | | Multiforme (GBM) | | | |
| NEO-100 | | | Neonc | Oncology | Lung Cancer | Preclinical | Global | G Protein-Coupled Receptor 78 |
| | | | Technologies, Inc. | | | | | (GPR78) Agonist; Ras Inhibitor |
| PLX-8394 | | | Plexxikon Inc. | Oncology | Anaplastic Thyroid | Phase II | United States | B-Raf Kinase Inhibitor |
| | | | | | Cancer; Bile | | | |
| | | | | | Duct Cancer | | | |
| | | | | | (Cholangiocarcinoma); | | | |
| | | | | | Colorectal Cancer; | | | |
| | | | | | Melanoma; | | | |
| | | | | | Non-Small | | | |
| | | | | | Cell Lung Cancer; | | | |
| | | | | | Papillary Thyroid | | | |
| | | | | | Cancer; | | | |
| | | | | | Solid Tumor | | | |
| PLX-8394 | | | Plexxikon Inc. | Oncology | Leukemias | Phase I | United States | B-Raf Kinase Inhibitor |
| PLX-8394 | | | Plexxikon Inc. | Oncology | Hairy Cell | Inactive | United States | B-Raf Kinase Inhibitor |
| | | | | | Leukemia | | | |
| RAF-265 | | | Novartis AG | Oncology | Metastatic Melanoma | Phase II | Global | B-Raf Kinase Inhibitor; C-Raf Kinase |
| | | | | | | | | Inhibitor; Vascular Endothelial Growth |
| | | | | | | | | Factor Receptor-2 (VEGFR-2) |
| | | | | | | | | Antagonist |
| RAF-265 | | | Novartis AG | Oncology | Solid Tumor | Inactive | Global | B-Raf Kinase Inhibitor; C-Raf Kinase |
| | | | | | | | | Inhibitor; Vascular Endothelial Growth |
| | | | | | | | | Factor Receptor-2 (VEGFR-2) |
| | | | | | | | | Antagonist |
| RXDX-105 | | | Ignyta, Inc. | Oncology | Colon Carcinoma; | Phase II | Global | B-Raf Kinase Inhibitor; Cytotoxic To |
| | | | | | Melanoma; Metastatic | | | Cells Expressing Epidermal Growth |
| | | | | | Colorectal Cancer; | | | Factor Receptor (EGFR, HER-1 or |
| | | | | | Solid Tumor | | | ErbB-1 or Proto-Oncogene c-ErbB-1 |
| | | | | | | | | or Receptor Tyrosine-Protein Kinase |
| | | | | | | | | erbB-1); Proto-Oncogene Tyrosine- |
| | | | | | | | | Protein Kinase ROS (Proto-Oncogene |
| | | | | | | | | c-Ros-1 or Receptor Tyrosine Kinase |
| | | | | | | | | c-Ros Oncogene 1 or c-Ros Receptor |
| | | | | | | | | Tyrosine Kinase or EC 2.7.10.1) |
| | | | | | | | | Inhibitor |
| TAK-580 | | | Millennium | Oncology | Metastatic Melanoma; | Phase II | Global | A-Raf Kinase Inhibitor; B-Raf Kinase |
| | | | Pharmaceuticals, Inc. | | Solid Tumor | | | Inhibitor; C-Raf Kinase Inhibitor |
| TAK-580 | | | Millennium | Oncology | Nonhematologic | Phase I | Global | A-Raf Kinase Inhibitor; B-Raf Kinase |
| | | | Pharmaceuticals, Inc. | | Malignancy | | | Inhibitor; C-Raf Kinase Inhibitor |
| ulixertinib | ulixertinib | | BioMed Valley | Oncology | Acute Myelocytic | Phase II | Global | Mitogen Activated Protein Kinase 1 |
| | [INN] | | Discoveries, Inc | | Leukemia | | | (MAP Kinase 1 or MAPK 1 or |
| | | | | | (AML, Acute | | | Extracellular Signal Regulated Kinase |
| | | | | | Myeloblastic | | | 2 or ERK-2 or Protein Tyrosine |
| | | | | | Leukemia); | | | Kinase ERK2 or EC 2.7.11.24) |
| | | | | | Advanced | | | Inhibitor; Mitogen Activated Protein |
| | | | | | Malignancy; | | | Kinase 3 (MAP Kinase 3 or MAPK 3 |
| | | | | | Colorectal Cancer; | | | or Extracellular Signal Regulated |
| | | | | | Melanoma; | | | Kinase 1 or ERK-1) Inhibitor |
| | | | | | Metastatic Cancer; | | | |
| | | | | | Myelodysplastic | | | |
| | | | | | Syndrome; | | | |
| | | | | | Non-Small | | | |
| | | | | | Cell Lung Cancer | | | |
| ulixertinib | ulixertinib | | BioMed Valley | Oncology | Metastatic | Preclinical | Global | Mitogen Activated Protein Kinase 1 |
| | [INN] | | Discoveries, Inc | | Adenocarcinoma | | | (MAP Kinase 1 or MAPK 1 or |
| | | | | | of The Pancreas; | | | Extracellular Signal Regulated Kinase |
| | | | | | Metastatic | | | 2 or ERK-2 or Protein Tyrosine |
| | | | | | Pancreatic Cancer; | | | Kinase ERK2 or EC 2.7.11.24) |
| | | | | | Pancreatic Cancer | | | Inhibitor; Mitogen Activated Protein |
| | | | | | | | | Kinase 3 (MAP Kinase 3 or MAPK 3 |
| | | | | | | | | or Extracellular Signal Regulated |
| | | | | | | | | Kinase 1 or ERK-1) Inhibitor |
| BAL-3833 | | | Basilea | Oncology | Metastatic Melanoma | Phase I | Global | A-Raf Kinase Inhibitor; B-Raf Kinase |
| | | | Pharmaceutica AG | | | | | Inhibitor; C-Raf Kinase Inhibitor |
| BGB-283 | | | BeiGene(Beijing) | Oncology | Colorectal Cancer; | Phase I | China | B-Raf Kinase Inhibitor; Epidermal |
| | | | Co., Ltd | | Endometrial | | | Growth Factor Receptor (EGFR, |
| | | | | | Cancer; | | | HER-1 or ErbB-1) Antagonist |
| | | | | | Melanoma; | | | |
| | | | | | Non-Small | | | |
| | | | | | Cell Lung | | | |
| | | | | | Cancer; Solid | | | |
| | | | | | Tumor; Thyroid | | | |
| | | | | | Cancer | | | |
| BGB-283 | | | Merck KGaA | Oncology | Solid Tumor | Phase I | Global | B-Raf Kinase Inhibitor; Epidermal |
| | | | | | | | | Growth Factor Receptor (EGFR, |
| | | | | | | | | HER-1 or ErbB-1) Antagonist |
| dabrafenib | dabrafenib | | AstraZeneca Plc | Oncology | Metastatic Melanoma | Phase I | Global | B-Raf Kinase Inhibitor; CD274 |
| mesylate + | mesylate + | | | | | | | (Programmed Cell Death 1-Ligand 1 |
| durvalumab + | durvalumab + | | | | | | | or PDL-1) Inhibitor; Mitogen Activated |
| trametinib | trametinib | | | | | | | Protein Kinase Kinase 1 (MEK-1 or |
| dimethyl | dimethyl | | | | | | | MAP2K1) Inhibitor; Mitogen Activated |
| sulfoxide | sulfoxide | | | | | | | Protein Kinase Kinase 2 (MEK-2 or |
| | | | | | | | | MAP2K2) Inhibitor |
| HM-95573 | | | Hanmi | Oncology | Melanoma; | Phase I | South Korea | Raf Kinase Inhibitor |
| | | | Pharmaceuticals, | | Solid Tumor | | | |
| | | | Co. Ltd. | | | | | |
| hydroxychloroquine + | hydroxychloroquine | | VG Life | Oncology | Breast Cancer; | Phase I | Global | B-Raf Kinase Inhibitor; c-kit Receptor |
| sorafenib | [INN] + | | Sciences, Inc. | | Lung | | | (SCFR, CD117) Antagonist; C-Raf |
| tosylate | sorafenib | | | | Adenocarcinoma; | | | Kinase Inhibitor; Platelet Derived |
| | tosylate | | | | Metastatic | | | Growth Factor Receptor Beta |
| | | | | | Ovarian Cancer; | | | (PDGFR-Beta) Antagonist; Proto- |
| | | | | | Solid Tumor | | | Oncogene Tyrosine-Protein Kinase |
| | | | | | | | | Receptor Ret (RET Receptor Tyrosine |
| | | | | | | | | Kinase or Cadherin Family Member |
| | | | | | | | | 12 or Proto-Oncogene c-Ret or EC |
| | | | | | | | | 2.7.10.1) Inhibitor; Receptor-Type |
| | | | | | | | | Tyrosine-Protein Kinase FLT3 (FMS- |
| | | | | | | | | Like Tyrosine Kinase 3 or FL Cytokine |
| | | | | | | | | Receptor or Stem Cell Tyrosine |
| | | | | | | | | Kinase 1 or CD135 or Fetal Liver |
| | | | | | | | | Kinase-2 or EC 2.7.10.1) Antagonist; |
| | | | | | | | | Vascular Endothelial Growth Factor |
| | | | | | | | | Receptor-1 (VEGFR-1) Antagonist; |
| | | | | | | | | Vascular Endothelial Growth Factor |
| | | | | | | | | Receptor-2 (VEGFR-2) Antagonist; |
| | | | | | | | | Vascular Endothelial Growth Factor |
| | | | | | | | | Receptor-3 (VEGFR-3) Antagonist |
| LY-3009120 | | | Eli Lilly | Oncology | Colorectal Cancer; | Phase I | Global | A-Raf Kinase Inhibitor; B-Raf Kinase |
| | | | and Company | | Malignant | | | Inhibitor; C-Raf Kinase Inhibitor |
| | | | | | Neoplasms; | | | |
| | | | | | Metastatic | | | |
| | | | | | Melanoma; | | | |
| | | | | | Non-Small | | | |
| | | | | | Cell Lung Cancer | | | |
| RG-7304 | | | Chugai | Oncology | Multiple Myeloma | Phase I | Japan | B-Raf Kinase Inhibitor; C-Raf Kinase |
| | | | Pharmaceutical | | (Kahler Disease); | | | Inhibitor; Mitogen Activated Protein |
| | | | Co., Ltd. | | Solid Tumor | | | Kinase Kinase 1 (MEK-1 or MAP2K1) |
| | | | | | | | | Inhibitor |
| RG-7304 | | | F. Hoffmann- | Oncology | Melanoma; | Phase I | Global | B-Raf Kinase Inhibitor; C-Raf Kinase |
| | | | La Roche Ltd. | | Non-Small | | | Inhibitor; Mitogen Activated Protein |
| | | | | | Cell Lung Cancer; | | | Kinase Kinase 1 (MEK-1 or MAP2K1) |
| | | | | | Pancreatic Cancer | | | Inhibitor |
| RG-7842 | | | Genentech, Inc. | Oncology | Solid Tumor | Phase I | Global | Mitogen Activated Protein Kinase 1 |
| | | | | | | | | (MAP Kinase 1 or MAPK 1 or |
| | | | | | | | | Extracellular Signal Regulated Kinase |
| | | | | | | | | 2 or ERK-2 or Protein Tyrosine |
| | | | | | | | | Kinase ERK2 or EC 2.7.11.24) |
| | | | | | | | | Inhibitor; Mitogen Activated Protein |
| | | | | | | | | Kinase 3 (MAP Kinase 3 or MAPK 3 |
| | | | | | | | | or Extracellular Signal Regulated |
| | | | | | | | | Kinase 1 or ERK-1) Inhibitor |
| salirasib | salirasib | | Ono | Oncology | Pancreatic Cancer; | Phase I | Japan | Ras Inhibitor |
| | | | Pharmaceutical | | Solid Tumor | | | |
| | | | Co., Ltd. | | | | | |
| salirasib | salirasib | | Kadmon | Oncology | Colorectal Cancer; | Inactive | Global | Ras Inhibitor |
| | | | Corporation, LLC | | Non-Small | | | |
| | | | | | Cell Lung Cancer; | | | |
| | | | | | Pancreatic Cancer | | | |
| AEZS-136 | | | Aeterna | Oncology | Solid Tumor | Preclinical | Global | Mitogen Activated Protein Kinase 1 |
| | | | Zentaris Inc. | | | | | (MAP Kinase 1 or MAPK 1 or |
| | | | | | | | | Extracellular Signal Regulated Kinase |
| | | | | | | | | 2 or ERK-2 or Protein Tyrosine |
| | | | | | | | | Kinase ERK2 or EC 2.7.11.24) |
| | | | | | | | | Inhibitor; Mitogen Activated Protein |
| | | | | | | | | Kinase 3 (MAP Kinase 3 or MAPK 3 |
| | | | | | | | | or Extracellular Signal Regulated |
| | | | | | | | | Kinase 1 or ERK-1) Inhibitor; |
| | | | | | | | | Phosphatidylinositol 3-Kinase (PI3K) |
| | | | | | | | | Inhibitor |
| ARI-4175 | | | Arisaph | Oncology | Colorectal Cancer; | Preclinical | Global | B-Raf Kinase Inhibitor; Dipeptidyl |
| | | | Pharmaceuticals, Inc. | | Metastatic | | | Peptidase-4 (DPP-4) Inhibitor |
| | | | | | Melanoma; | | | |
| | | | | | Sarcomas | | | |
| ASN-003 | | | Asana | Oncology | Melanoma | Preclinical | Global | B-Raf Kinase Inhibitor |
| | | | BioSciences, LLC | | | | | |
| CCT-196969 | | | Basilea | Oncology | Melanoma | Preclinical | Global | B-Raf Kinase Inhibitor; c-Src |
| | | | Pharmaceutica AG | | | | | Tyrosine Kinase Inhibitor; Lck |
| | | | | | | | | Tyrosine Kinase Inhibitor; Mitogen |
| | | | | | | | | Activated Protein Kinase Kinase |
| | | | | | | | | (MEK or MAP2K) Inhibitor; Mitogen |
| | | | | | | | | Activated Protein Kinase Kinase |
| | | | | | | | | Kinase (cMos or cRaf or MAPKKK or |
| | | | | | | | | MAP3K or MAP Kinase Kinase |
| | | | | | | | | Kinase or EC 2.7.11.25) Inhibitor; p38 |
| | | | | | | | | MAP Kinase Inhibitor; Tyrosine- |
| | | | | | | | | Protein Kinase Mer (Proto-Oncogene |
| | | | | | | | | c-Mer) Antagonist |
| CCT-241161 | | | Basilea | Oncology | Melanoma | Preclinical | Global | B-Raf Kinase Inhibitor; c-Src |
| | | | Pharmaceutica AG | | | | | Tyrosine Kinase Inhibitor; Lck |
| | | | | | | | | Tyrosine Kinase Inhibitor; Mitogen |
| | | | | | | | | Activated Protein Kinase Kinase |
| | | | | | | | | (MEK or MAP2K) Inhibitor; Mitogen |
| | | | | | | | | Activated Protein Kinase Kinase |
| | | | | | | | | Kinase (cMos or cRaf or MAPKKK or |
| | | | | | | | | MAP3K or MAP Kinase Kinase |
| | | | | | | | | Kinase or EC 2.7.11.25) Inhibitor; p38 |
| | | | | | | | | MAP Kinase Inhibitor; Tyrosine- |
| | | | | | | | | Protein Kinase Mer (Proto-Oncogene |
| | | | | | | | | c-Mer) Antagonist |
| CS-410 | | | Chipscreen | Cardiovascular | Cardiovascular | Preclinical | Global | Mitogen Activated Protein Kinase |
| | | | Biosciences Ltd | | Disease | | | Kinase Kinase 5 (Apoptosis Signal |
| | | | | | | | | Regulating Kinase 1 or MAPK/ERK |
| | | | | | | | | Kinase Kinase 5 or MEK Kinase 5 or |
| | | | | | | | | ASK-1 or MAP3K5 or EC 2.7.11.25) |
| | | | | | | | | Inhibitor |
| CS-410 | | | Chipscreen | Central Nervous | Neurodegenerative | Preclinical | Global | Mitogen Activated Protein Kinase |
| | | | Biosciences Ltd | Systern | Diseases | | | Kinase Kinase 5 (Apoptosis Signal |
| | | | | | | | | Regulating Kinase 1 or MAPK/ERK |
| | | | | | | | | Kinase Kinase 5 or MEK Kinase 5 or |
| | | | | | | | | ASK-1 or MAP3K5 or EC 2.7.11.25) |
| | | | | | | | | Inhibitor |
| CS-410 | | | Chipscreen | Oncology | Gastric Cancer | Preclinical | Global | Mitogen Activated Protein Kinase |
| | | | Biosciences Ltd | | | | | Kinase Kinase 5 (Apoptosis Signal |
| | | | | | | | | Regulating Kinase 1 or MAPK/ERK |
| | | | | | | | | Kinase Kinase 5 or MEK Kinase 5 or |
| | | | | | | | | ASK-1 or MAP3K5 or EC 2.7.11.25) |
| | | | | | | | | Inhibitor |
| CT-207 | | | HEC Pharm | Oncology | Melanoma | Preclinical | Global | B-Raf Kinase Inhibitor |
| | | | Co., Ltd. | | | | | |
| CT-317 | | | HEC Pharm | Oncology | Melanoma | Preclinical | Global | B-Raf Kinase Inhibitor |
| | | | Co., Ltd. | | | | | |
| Drugs to | | | Ruga | Oncology | | Preclinical | Global | B-Raf Kinase Inhibitor |
| Inhibit | | | Corporation | | | | | |
| B-Raf | | | | | | | | |
| Kinase for | | | | | | | | |
| Cancer | | | | | | | | |
| EBI-907 | | | Eternity | Oncology | Melanoma; Metastatic | Preclinical | Global | B-Raf Kinase Inhibitor |
| | | | Bioscience Inc. | | Colorectal Cancer | | | |
| EBI-945 | | | Eternity | Oncology | | Preclinical | Global | B-Raf Kinase Inhibitor |
| | | | Bioscience Inc. | | | | | |
| KO-947 | | | Kura | Oncology | Colorectal Cancer; | Preclinical | Global | Mitogen Activated Protein Kinase 1 |
| | | | Oncology, Inc. | | Melanoma; Non-Small | | | (MAP Kinase 1 or MAPK 1 or |
| | | | | | Cell Lung Cancer; | | | Extracellular Signal Regulated Kinase |
| | | | | | Pancreatic Cancer | | | 2 or ERK-2 or Protein Tyrosine |
| | | | | | | | | Kinase ERK2 or EC 2.7.11.24) |
| | | | | | | | | Inhibitor; Mitogen Activated Protein |
| | | | | | | | | Kinase 3 (MAP Kinase 3 or MAPK 3 |
| | | | | | | | | or Extracellular Signal Regulated |
| | | | | | | | | Kinase 1 or ERK-1) Inhibitor |
| LXH-254 | | | Novartis AG | Oncology | Solid Tumor | Preclinical | Global | A-Raf Kinase Inhibitor; B-Raf Kinase |
| | | | | | | | | Inhibitor; C-Raf Kinase Inhibitor |
| MDC-1016 | | | Medicon | Oncology | Pancreatic Tumor | Preclinical | Global | Ras Inhibitor |
| | | | Pharmaceuticals, Inc | | | | | |
| MT-477 | | | Medisyn | Oncology | Lung | Preclinical | Global | Protein Kinase C Alpha (PKC-Alpha) |
| | | | Technologies, Inc. | | Adenocarcinoma; | | | Inhibitor; Ras Inhibitor |
| | | | | | Pancreatic Ductal | | | |
| | | | | | Adenocarcinoma | | | |
| NCB-0594 | | | Carna | Oncology | | Preclinical | Global | TRAF2 and NCK-Interacting Protein |
| | | | Biosciences, Inc. | | | | | Kinase (TNIK) Inhibitor |
| NCB-0846 | | | Carna | Oncology | Colon Cancer | Preclinical | Global | TRAF2 and NCK-Interacting Protein |
| | | | Biosciences, Inc. | | | | | Kinase (TNIK) Inhibitor |
| NMSP-285 | | | Nerviano | Oncology | | Preclinical | Global | B-Raf Kinase Inhibitor |
| | | | Medical | | | | | |
| | | | Sciences S.r.I. | | | | | |
| ON-108600 | | | Onconova | Oncology | Breast Cancer; | Preclinical | Global | Casein Kinase 2 Inhibitor; TRAF2 |
| | | | Therapeutics, Inc. | | Solid Tumor | | | and NCK-Interacting Protein Kinase |
| | | | | | | | | (TNIK) Inhibitor |
| PV-103 | | | PepVax, Inc. | Oncology | Colorectal Cancer; | Preclinical | Global | B-Raf Kinase Inhibitor |
| | | | | | Melanoma | | | |
| RX-8243 | | | Rexahn | Oncology | Colorectal Cancer; | Preclinical | Global | Aurora A Kinase Inhibitor; Mitogen- |
| | | | Pharmaceuticals, Inc. | | Ovarian Cancer | | | Activated Protein (MAP) Kinase |
| | | | | | | | | Inhibitor; p38 MAP Kinase Inhibitor; |
| | | | | | | | | Protein Kinase B (PKB or Akt) |
| | | | | | | | | Inhibitor; Ras Inhibitor |
| Small | | | Genentech, Inc. | Oncology | | Preclinical | Global | Mitogen-Activated Protein Kinase |
| Molecule to | | | | | | | | Kinase Kinase Kinase 4 (HPK/GCK- |
| Inhibit | | | | | | | | Like Kinase HGK or MAPK/ERK |
| MAP4K4 for | | | | | | | | Kinase Kinase Kinase 4 or MEK |
| Cancer | | | | | | | | Kinase Kinase 4 or MEKKK 4 or |
| | | | | | | | | MAP4K4 or EC 2.7.11.1) Inhibitor |
| Small | | | Novartis AG | Oncology | | Preclinical | Global | A-Raf Kinase Inhibitor; B-Raf Kinase |
| Molecules to | | | | | | | | Inhibitor; C-Raf Kinase Inhibitor |
| Inhibit | | | | | | | | |
| pan-RAF | | | | | | | | |
| Kinase for | | | | | | | | |
| Oncology | | | | | | | | |
| Small | | | Amitech | Oncology | Pancreatic | Preclinical | Global | B-Raf Kinase Inhibitor; C-Raf Kinase |
| Molecules to | | | Therapeutic | | Cancer; | | | Inhibitor |
| Inhibit | | | Solutions, Inc. | | Solid Tumor | | | |
| Raf | | | | | | | | |
| Kinases for | | | | | | | | |
| Cancer | | | | | | | | |
| STP-503 | | Trisilensa | Sirnaomics, Inc. | Oncology | Breast Cancer; | Preclinical | Global | C-Raf Kinase Inhibitor; Epidermal |
| | | | | | Lung Cancer | | | Growth Factor Receptor (EGFR, |
| | | | | | | | | HER-1 or ErbB-1) Antagonist; |
| | | | | | | | | Serine/Threonine-Protein Kinase |
| | | | | | | | | mTOR (Mammalian Target Of |
| | | | | | | | | Rapamycin or Mechanistic Target of |
| | | | | | | | | Rapamycin or EC 2.7.11.1) Inhibitor |
| TAK-632 | | | Takeda | Oncology | Melanoma | Preclinical | Global | B-Raf Kinase Inhibitor |
| | | | Pharmaceutical | | | | | |
| | | | Company Limited | | | | | |
| TEW-0201 | | | MedPacto, Inc. | Oncology | Colorectal Cancer; | Preclinical | Global | B-Raf Kinase Inhibitor; C-Raf Kinase |
| | | | | | Melanoma | | | Inhibitor; Raf Kinase Inhibitor |
| AIK-4 | | | Allinky Biopharma | Oncology | Colorectal Cancer; | Discovery | Global | Ras Inhibitor |
| | | | | | Lung Cancer; | | | |
| | | | | | Non-Hodgkin | | | |
| | | | | | Lymphoma; | | | |
| | | | | | Pancreatic Cancer | | | |
| AR-00457679 | | | Array | Oncology | | Discovery | Global | B-Raf Kinase Inhibitor |
| | | | BioPharma Inc. | | | | | |
| CB-745 | | | AGV | Oncology | Colon Cancer; | Discovery | Global | Mitogen Activated Protein Kinase 1 |
| | | | Discovery, SAS | | Metastatic Melanoma; | | | (MAP Kinase 1 or MAPK 1 or |
| | | | | | Pancreatic Cancer | | | Extracellular Signal Regulated Kinase |
| | | | | | | | | 2 or ERK-2 or Protein Tyrosine |
| | | | | | | | | Kinase ERK2 or EC 2.7.11.24) |
| | | | | | | | | Inhibitor; Mitogen Activated Protein |
| | | | | | | | | Kinase 3 (MAP Kinase 3 or MAPK 3 |
| | | | | | | | | or Extracellular Signal Regulated |
| | | | | | | | | Kinase 1 or ERK-1) Inhibitor |
| HD-001 | | | AstraZeneca Plc | Oncology | | Discovery | Global | GTPase KRas (K-Ras 2 or Ki-Ras or |
| | | | | | | | | c-K-Ras or c-Ki-Ras or EC 3.6.5.2) |
| | | | | | | | | Inhibitor |
| SCH-722984 | | | Merck & Co., Inc. | Oncology | Melanoma | Discovery | Global | Mitogen Activated Protein Kinase 3 |
| | | | | | | | | (MAP Kinase 3 or MAPK 3 or |
| | | | | | | | | Extracellular Signal Regulated Kinase |
| | | | | | | | | 1 or ERK-1) Inhibitor |
| Small | | | Aurigene | Oncology | | Discovery | Global | GTPase KRas (K-Ras 2 or Ki-Ras or |
| Molecule to | | | Discovery | | | | | c-K-Ras or c-Ki-Ras or EC 3.6.5.2) |
| Inhibit | | | Technologies | | | | | Inhibitor |
| K-RAS for | | | Limited | | | | | |
| Oncology | | | | | | | | |
| Small | | | Sareum | Oncology | | Discovery | Global | B-Raf Kinase Inhibitor |
| Molecules to | | | Holdings Plc | | | | | |
| Inhibit | | | | | | | | |
| B-RAF | | | | | | | | |
| Kinase for | | | | | | | | |
| Oncology | | | | | | | | |
| Small | | | Aeterna | Oncology | | Discovery | Global | Aurora B Kinase Inhibitor; Mitogen |
| Molecules to | | | Zentaris Inc. | | | | | Activated Protein Kinase 1 (MAP |
| Inhibit | | | | | | | | Kinase 1 or MAPK 1 or Extracellular |
| ERK2 and | | | | | | | | Signal Regulated Kinase 2 or ERK-2 |
| Aurora B | | | | | | | | or Protein Tyrosine Kinase ERK2 or |
| Kinase for | | | | | | | | EC 2.7.11.24) Inhibitor |
| Cancer | | | | | | | | |
| Small | | | Araxes | Oncology | | Discovery | Global | GTPase KRas (K-Ras 2 or Ki-Ras or |
| Molecules to | | | Pharma LLC | | | | | c-K-Ras or c-Ki-Ras or EC 3.6.5.2) |
| Inhibit | | | | | | | | Inhibitor |
| K-Ras for | | | | | | | | |
| Cancer | | | | | | | | |
| Small | | | Boehringer | Oncology | | Discovery | Global | GTPase KRas (K-Ras 2 or Ki-Ras or |
| Molecules to | | | Ingelheim GmbH | | | | | c-K-Ras or c-Ki-Ras or EC 3.6.5.2) |
| Inhibit | | | | | | | | Inhibitor |
| K-Ras for | | | | | | | | |
| Oncology | | | | | | | | |
| Small | | | NantBioScience | Oncology | | Discovery | Global | GTPase KRas (K-Ras 2 or Ki-Ras or |
| Molecules to | | | | | | | | c-K-Ras or c-Ki-Ras or EC 3.6.5.2) |
| Inhibit | | | | | | | | Inhibitor |
| KRAS for | | | | | | | | |
| Cancer | | | | | | | | |
| Small | | | Nimbus | Oncology | Solid Tumor | Discovery | Global | GTPase KRas (K-Ras 2 or Ki-Ras or |
| Molecules to | | | Therapeutics, LLC | | | | | c-K-Ras or c-Ki-Ras or EC 3.6.5.2) |
| Inhibit | | | | | | | | Inhibitor |
| KRas for | | | | | | | | |
| Solid Tumor | | | | | | | | |
| Small | | | Redx | Oncology | Colorectal Cancer | Discovery | Global | A-Raf Kinase Inhibitor; B-Raf Kinase |
| Molecules to | | | Pharma Plc | | | | | Inhibitor; C-Raf Kinase Inhibitor |
| Inhibit | | | | | | | | |
| Pan-Raf | | | | | | | | |
| Kinases for | | | | | | | | |
| Colorectal Cancer | | | | | | | | |
| Small | | | Astex | Oncology | | Discovery | Global | TRAF2 and NCK-Interacting Protein |
| Molecules to | | | Pharmaceuticals, Inc. | | | | | Kinase (TNIK) Inhibitor |
| Inhibit | | | | | | | | |
| TNIK for | | | | | | | | |
| Oncology | | | | | | | | |
| Synthetic | | | PeptiDream Inc. | Oncology | | Discovery | Global | GTPase KRas (K-Ras 2 or Ki-Ras or |
| Peptides to | | | | | | | | c-K-Ras or c-Ki-Ras or EC 3.6.5.2) |
| Inhibit | | | | | | | | Inhibitor |
| KRAS for | | | | | | | | |
| Oncology | | | | | | | | |
| ARQ-736 | | | ArQule, Inc. | Oncology | Solid Tumor | Inactive | Global | A-Raf Kinase Inhibitor; B-Raf Kinase |
| | | | | | | | | Inhibitor; C-Raf Kinase Inhibitor; Raf |
| | | | | | | | | Kinase Inhibitor |
| CKBP-002 | | | CK Life Sciences Int'l., | Oncology | Liver Cancer; | Inactive | Global | C-Raf Kinase Inhibitor; Mitogen |
| | | | (Holdings) Inc. | | Pancreatic Cancer; | | | Activated Protein Kinase Kinase |
| | | | | | Solid Tumor | | | (MEK or MAP2K) Inhibitor; Mitogen- |
| | | | | | | | | Activated Protein (MAP) Kinase |
| | | | | | | | | Inhibitor; Signal Transducer And |
| | | | | | | | | Activator Of Transcription 3 (STAT3) |
| | | | | | | | | Inhibitor |
| DP-2514 | | | Eli Lilly | Oncology | Melanoma | Inactive | Global | B-Raf Kinase Inhibitor |
| | | | and Company | | | | | |
| DP-3346 | | | Eli Lilly | Oncology | Melanoma | Inactive | Global | B-Raf Kinase Inhibitor |
| | | | and Company | | | | | |
| Fluorapacin | | | ACEA | Oncology | Advanced Malignancy | Inactive | Global | Mitogen Activated Protein Kinase 3 |
| | | | Biosciences, Inc. | | | | | (MAP Kinase 3 or MAPK 3 or |
| | | | | | | | | Extracellular Signal Regulated Kinase |
| | | | | | | | | 1 or ERK-1) Inhibitor; Mitogen |
| | | | | | | | | Activated Protein Kinase Kinase 1 |
| | | | | | | | | (MEK-1 or MAP2K1) Inhibitor |
| GDC-0879 | | | Genentech, Inc. | Oncology | Melanoma | Inactive | Global | B-Raf Kinase Inhibitor |
| LE-rafAON | | | Insys | Oncology | Solid Tumor | Inactive | Global | C-Raf Kinase Inhibitor |
| | | | Therapeutics, Inc. | | | | | |
| MK-8353 | | | Merck & Co., Inc. | Oncology | Metastatic Colorectal | Inactive | Global | Mitogen Activated Protein Kinase 3 |
| | | | | | Cancer; Metastatic | | | (MAP Kinase 3 or MAPK 3 or |
| | | | | | Melanoma; Solid Tumor | | | Extracellular Signal Regulated Kinase |
| | | | | | | | | 1 or ERK-1) Inhibitor |
| NMSP-383 | | | Nerviano Medical | Oncology | Melanoma | Inactive | Global | B-Raf Kinase Inhibitor |
| | | | Sciences S.r.I. | | | | | |
| NMSP-730 | | | Nerviano Medical | Oncology | Melanoma | Inactive | Global | B-Raf Kinase Inhibitor |
| | | | Sciences S.r.I. | | | | | |
| PLX-4720 | | | Plexxikon Inc. | Musculoskeletal | Cachexia | Inactive | Global | B-Raf Kinase Inhibitor |
| | | | | Disorders | | | | |
| PLX-4720 | | | Plexxikon Inc. | Oncology | Anaplastic | Inactive | Global | B-Raf Kinase Inhibitor |
| | | | | | Thyroid Cancer | | | |
| PNT-300 | | | ProNAi | Oncology | | Inactive | Global | GTPase KRas (K-Ras 2 or Ki-Ras or |
| | | | Therapeutics, Inc. | | | | | c-K-Ras or c-Ki-Ras or EC 3.6.5.2) |
| | | | | | | | | Inhibitor |
| SCH-772984 | | | Merck & Co., Inc. | Oncology | Solid Tumor | Inactive | Global | Mitogen Activated Protein Kinase 3 |
| | | | | | | | | (MAP Kinase 3 or MAPK 3 or |
| | | | | | | | | Extracellular Signal Regulated Kinase |
| | | | | | | | | 1 or ERK-1) Inhibitor |
| Small | | | Locus | Oncology | | Inactive | Global | C-Raf Kinase Inhibitor |
| Molecule to | | | Pharmaceuticals, Inc. | | | | | |
| Inhibit | | | (Inactive) | | | | | |
| C-Raf for | | | | | | | | |
| Oncology | | | | | | | | |
| Small | | | TetraGene, LLC | Oncology | Multiple Myeloma | Inactive | Global | GTPase KRas (K-Ras 2 or Ki-Ras or |
| Molecule to | | | | | (Kahler Disease) | | | c-K-Ras or c-Ki-Ras or EC 3.6.5.2) |
| Inhibit | | | | | | | | Inhibitor |
| K-Ras for | | | | | | | | |
| Multiple Myeloma | | | | | | | | |
| Small | | | Redx Pharma Plc | Oncology | Colorectal Cancer; | Inactive | Global | B-Raf Kinase Inhibitor; C-Raf Kinase |
| Molecules to | | | | | Melanoma | | | Inhibitor |
| Inhibit | | | | | | | | |
| B-raf/C-Raf for | | | | | | | | |
| Cancer | | | | | | | | |
| Small | | | Astex | Oncology | | Inactive | Global | B-Raf Kinase Inhibitor |
| Molecules to | | | Pharmaceuticals, Inc. | | | | | |
| Inhibit | | | | | | | | |
| BRAF for | | | | | | | | |
| Oncology | | | | | | | | |
| Small | | | Kura | Oncology | Solid Tumor | Inactive | Global | Mitogen Activated Protein Kinase 1 |
| Molecules to | | | Oncology, Inc. | | | | | (MAP Kinase 1 or MAPK 1 or |
| Inhibit | | | | | | | | Extracellular Signal Regulated Kinase |
| ERK-1 and | | | | | | | | 2 or ERK-2 or Protein Tyrosine |
| ERK-2 for | | | | | | | | Kinase ERK2 or EC 2.7.11.24) |
| Solid Tumors | | | | | | | | Inhibitor; Mitogen Activated Protein |
| | | | | | | | | Kinase 3 (MAP Kinase 3 or MAPK 3 |
| | | | | | | | | or Extracellular Signal Regulated |
| | | | | | | | | Kinase 1 or ERK-1) Inhibitor |
| Small | | | Navigen | Oncology | | Inactive | Global | GTPase KRas (K-Ras 2 or Ki-Ras or |
| Molecules to | | | Pharmaceuticals, Inc. | | | | | c-K-Ras or c-Ki-Ras or EC 3.6.5.2) |
| Inhibit | | | | | | | | Inhibitor |
| K-Ras for | | | | | | | | |
| Oncology | | | | | | | | |
| Small | | | CrystalGenomics, Inc. | Oncology | | Inactive | Global | TRAF2 and NCK-Interacting Protein |
| Molecules to | | | | | | | | Kinase (TNIK) Inhibitor |
| Inhibit | | | | | | | | |
| TNIK for | | | | | | | | |
| Cancer | | | | | | | | |
| Tumor Adaptive | | | Ruga Corporation | Oncology | Metastatic | Inactive | Global | B-Raf Kinase Inhibitor |
| Responses Program | | | | | Ovarian Cancer | | | |
| AEZS-131 | | | AEterna | Oncology | Breast Cancer; | Discontinued | Global | Mitogen Activated Protein Kinase 3 |
| | | | Zentaris Inc. | | Colon Cancer | | | (MAP Kinase 3 or MAPK 3 or |
| | | | | | | | | Extracellular Signal Regulated Kinase |
| | | | | | | | | 1 or ERK-1) Inhibitor; Mitogen |
| | | | | | | | | Activated Protein Kinase Kinase 1 |
| | | | | | | | | (MEK-1 or MAP2K1) Inhibitor |
| ISIS-5132 | | | Ionis | Oncology | Epithelial Ovarian | Discontinued | Global | C-Raf Kinase Inhibitor |
| | | | Pharmaceuticals, Inc. | | Cancer; Hormone | | | |
| | | | | | Refractory (Castration | | | |
| | | | | | Resistant, Androgen- | | | |
| | | | | | Independent) | | | |
| | | | | | Prostate Cancer; | | | |
| | | | | | Metastatic Breast | | | |
| | | | | | Cancer; Metastatic | | | |
| | | | | | Colorectal Cancer | | | |
| PLX-3603 | | | Plexxikon Inc. | Oncology | Solid Tumor | Discontinued | Global | B-Raf Kinase Inhibitor |
| XL-281 | | | Exelixis, Inc. | Oncology | Colorectal Cancer; | Discontinued | Global | B-Raf Kinase Inhibitor; C-Raf Kinase |
| | | | | | Melanoma; Solid Tumor | | | Inhibitor |
| MM-41 | | | University | Oncology | Pancreatic Cancer | Preclinical | Global | Bcl-2 Inhibitor; GTPase KRas (K-Ras |
| | | | College London | | | | | 2 or Ki-Ras or c-K-Ras or c-Ki-Ras or |
| | | | | | | | | EC 3.6.5.2) Inhibitor |
| Oligonucleotide to | | | UNC Lineberger | Oncology | Colon Cancer; | Preclinical | Global | GTPase KRas (K-Ras 2 or Ki-Ras or |
| Inhibit | | | Comprehensive | | Lung Cancer | | | c-K-Ras or c-Ki-Ras or EC 3.6.5.2) |
| KRAS for | | | Cancer Center | | | | | Inhibitor |
| Cancer | | | | | | | | |
| Antisense RNAi | | | Brigham and | Oncology | | Discovery | Global | GTPase KRas (K-Ras 2 or Ki-Ras or |
| Oligonucleotides to | | | Women's Hospital | | | | | c-K-Ras or c-Ki-Ras or EC 3.6.5.2) |
| Inhibit | | | | | | | | Inhibitor |
| KRAS for | | | | | | | | |
| Cancer | | | | | | | | |
| Monoclonal | | | Vanderbilt University | Oncology | | Discovery | Global | B-Raf Kinase Inhibitor |
| Antibody to | | | | | | | | |
| Target B-Raf | | | | | | | | |
| Kinase for | | | | | | | | |
| Oncology | | | | | | | | |
| Small | | | Institut Curie | Oncology | | Discovery | Global | Mitogen-Activated Protein Kinase |
| Molecule to | | | | | | | | Kinase Kinase Kinase 2 (Germinal |
| Inhibit | | | | | | | | Center Kinase or MAPK/ERK Kinase |
| GCK for | | | | | | | | Kinase Kinase 2) Inhibitor |
| Oncology | | | | | | | | |
| Small | | | Vanderbilt University | Oncology | | Discovery | Global | GTPase KRas (K-Ras 2 or Ki-Ras or |
| Molecules to | | | | | | | | c-K-Ras or c-Ki-Ras or EC 3.6.5.2) |
| Inhibit | | | | | | | | Inhibitor |
| K-Ras for | | | | | | | | |
| Oncology | | | | | | | | |
| Small | | | University of Texas | Oncology | | Discovery | Global | Ras Inhibitor |
| Molecules to | | | Health Science | | | | | |
| Inhibit | | | Center at Houston | | | | | |
| Ras for | | | | | | | | |
| Oncology | | | | | | | | |
| SML-8731 | | | Dana- | Oncology | | Discovery | Global | GTPase KRas (K-Ras 2 or Ki-Ras or |
| | | | Farber Cancer | | | | | c-K-Ras or c-Ki-Ras or EC 3.6.5.2) |
| | | | Institute, Inc. | | | | | Inhibitor |

By “EMT pathway inhibitor” is meant a therapeutic agent, such as a pharmaceutical drug, that acts to inhibit the epithelial-mesenchymal transition (EMT). The inhibitor may be specific for the EMT pathway. Thus, in certain embodiments the EMT pathway inhibitor is not a multi-pathway inhibitor. In certain embodiments, the EMT pathway inhibitor is selected from Table I.

In further embodiments, the EMT pathway inhibitor is an FKBP-L polypeptide or a biologically active peptide fragment thereof. In preferred embodiments, the biologically active peptide fragment of FKBP-L comprises the amino acid sequence IRQQPRDPPTETLELEVSPDPAS (SEQ ID NO:791; referred to herein also as ALM201), or a sequence at least 90% identical thereto. In further embodiments, the FKBP-L polypeptide comprises the amino acid sequence shown as SEQ ID NO:789 or SEQ ID NO:790, or a sequence at least 90% identical thereto. In further embodiments, the biologically active peptide fragment of FKBP-L comprises the amino acid sequence shown as any one of SEQ ID Nos 792 to 811, or a sequence at least 90% identical thereto.

As used herein, the term “biologically active FKBP-L peptide” (e.g., fragment and/or modified polypeptides) is used to refer to a peptide or polypeptide that displays the same or similar amount and type of activity as the full-length FKBP-L polypeptide. In this context “biological activity” of an FKBP-L polypeptide, fragment or derivative refers to the ability to inhibit and/or reverse the EMT pathway (and/or the ability to down-regulate the MAPK pathway). MAPK is known to induce EMT via phosphorylation of the SNAIL/SLUG transcription factors, (Virtakoivu et al., 2015).

Biological activity of FKBP-L fragments or derivatives may be tested in comparison to full length FKBP-L using any of the in vitro or in vivo assays described in the accompanying examples, including cell-based assays of the mesenchymal phenotype, such as for example the colony formation assay, migration assay or invasion assay. In other embodiments, “biological activity” of an FKBP-L polypeptide, fragment or derivative may be demonstrated by assaying expression of one or more biomarkers of the EMT pathway (e.g. mesenchymal markers), or one or more biomarkers of the MAPK pathway.

The term “FKBP-L” refers to the protein FK506 binding protein-like, (McKeen et al. Endocrinology, 2008, Vol 149(11), 5724-34; Gene ID: 63943). FKBP-L and peptide fragments thereof have previously been demonstrated to possess potent anti-angiogenic activity (WO 2007/141533). The anti-angiogenic activity of FKBP-L peptide fragments appears to be dependent on an amino acid sequence located between amino acids 34-57, in the N-terminal region of the full-length protein. This anti-angiogenic activity suggested a clinical utility of the peptide in the treatment of cancers, particularly solid tumours.

The expression “FKBP-L polypeptide” is used in the specification according to its broadest meaning. It designates the naturally occurring full-length protein as shown in SEQ ID NO:789, together with homologues due to polymorphisms, other variants, mutants and portions of said polypeptide which retain their biological activities. For example, in certain embodiments, the FKBP-L polypeptide comprises SEQ ID NO:789 (GENBank Accession No. NP_071393; NM_022110; [gi:34304364]), or SEQ ID NO:790 with a Threonine at position 181 and a Glycine at position 186 of the wild-type sequence. Example constructs of other FKBP-L polypeptides (e.g., fragments and other modifications) and polynucleotide constructs encoding for FKBP-L polypeptides are described in WO 2007/141533, the contents of which are incorporated herein in their entirely by reference, expressly for this purpose.

In SEQ ID NO: 790, the FKBP-L insert (originally cloned into PUC18 by Cambridge Bioscience and now cloned into pcDNA3.1); had two inserted point mutations compared to the sequence that is deposited on the PUBMED database (SEQ ID NO: 789). There is a point mutation at 540 bp (from start codon): TCT to ACT which therefore converts a serine (S) to a Threonine (T) (amino acid: 181). There is also a point mutation at 555 bp (from start codon): AGG to GGG which therefore converts an Arginine (R) to a Glycine (G) (amino acid: 186). Both FKBP-L polypeptides (SEQ ID NO: 789 and SEQ ID NO: 790) display biological activity.

An FKBP-L polypeptide or peptide may include natural and/or chemically synthesized or artificial FKBP-L peptides, peptide mimetics, modified peptides (e.g., phosphopeptides, cyclic peptides, peptides containing D- and unnatural amino-acids, stapled peptides, peptides containing radiolabels), or peptides linked to antibodies, carbohydrates, monosaccharides, oligosaccharides, polysaccharides, glycolipids, heterocyclic compounds, nucleosides or nucleotides or parts thereof, and/or small organic or inorganic molecules (e.g., peptides modified with PEG or other stabilizing groups). Thus, the FKBP-L (poly)peptides of the invention also include chemically modified peptides or isomers and racemic forms.

As described herein, the methods and therapeutic agents for use according to the present invention may utilize a full-length FKBP-L polypeptide, or biologically active fragments of the polypeptide. Thus, certain embodiments of the present invention comprise a FKBP-L derivative which comprises or consists of a biologically active portion of the N-terminal amino acid sequence of naturally occurring FKBP-L. This sequence may comprise, consist essentially of, or consist of an active N-terminal portion of the FKBP-L polypeptide. In alternate embodiments, the polypeptide may comprise, consist essentially of, or consist of amino acids 1 to 57 of SEQ ID NO: 790 (i.e., SEQ ID NO: 796), or amino acids 34-57 of SEQ ID NO:790 (i.e., SEQ ID NO: 792), or amino acids 35-57 of SEQ ID NO:790 (i.e. SEQ ID NO:791). Or, the peptide may comprise, consist essentially of, or consist of a sequence that comprises at least 18 contiguous amino acids of SEQ ID NO: 792 (e.g., SEQ ID NOs: 798, 800, or 807). In alternate embodiments, the polypeptide used in the methods and compositions of the present invention may comprise, consist essential of, or consist of one of the amino acid sequences shown in any one of SEQ ID NOs: 789-811. In certain embodiments, the present invention comprises a biologically active fragment of FKBP-L, wherein said polypeptide includes no more than 200 consecutive amino acids of the amino acid sequence shown in SEQ ID NO:789, or SEQ ID NO:790, with the proviso that said polypeptide includes the amino acid sequence shown as SEQ ID NO:791.

As described herein, the peptides may be modified (e.g., to contain PEG and/or His tags, albumin conjugates or other modifications). Or, the present invention may comprise isolated polypeptides having a sequence at least 70%, or 75%, or 80%, or 85%, or 90%, or 95%, or 96%, or 97%, or 98%, or 99% identical to the amino acid sequences as set forth in any one of SEQ ID NOS: 789-811, including in particular sequences at least 70%, or 75%, or 80%, or 85%, or 90%, or 95%, or 96%, or 97%, or 98%, or 99% identical to the amino acid sequence shown as SEQ ID NO:791. In this regard, deliberate amino acid substitutions may be made in the peptide on the basis of similarity in polarity, charge, solubility, hydrophobicity, or hydrophilicity of the residues, as long as the specific biological activity (i.e. function) of the peptide is retained. The FKBP-L peptide may be of variable length as long as it retains its biological activity and can be used according to the various aspects of the invention described above.

Certain regions of the N-terminus of the FKBP-L protein may display biological activity, therefore the invention encompasses biologically active fragments of FKBP-L, in particular any fragment which exhibits biological activity substantially equivalent to that of the 23-mer peptide (SEQ ID NO:791). In certain embodiments, the biological activity of the FKBP-L 23mer peptide (SEQ ID NO:791; referred to herein also as ALM201) is exhibited as a reduction in expression of mesenchymal markers in Kuramochi cells or OVCAR3 cisplatin resistant cells. In further embodiments, the biological activity of the FKBP-L 23mer peptide (SEQ ID NO:791; referred to herein also as ALM201) is exhibited as a reversal of the mesenchymal phenotype in OVCAR3 or OVCAR4 cisplatin resistant cells.

A “fragment” of a FKBP-L polypeptide means an isolated peptide comprising a contiguous sequence of at least 6 amino acids, preferably at least 10 amino acids, or at least 15 amino acids, or at least 20 amino acids, or at least 23 amino acids of FKBP-L. The “fragment” preferably contains no more than 50, or no more than 45, or no more than 40, or no more than 35, or no more than 30, or no more than 25, or no more than 23 contiguous amino acids of FKBP-L. Preferred fragments for use according to the invention are those having the amino acid sequences shown in any one of SEQ ID Nos: 792-811, or minor sequence variants thereof (e.g. variants containing one or more conservative amino acid substitutions).

In certain embodiments the EMT pathway inhibitor (specifically) inhibits Vimentin, AXL, TWIST1, SNAIL and/or SLUG.

The EMT pathway inhibitor may be used together with a platinum-based chemotherapeutic agent as a first line treatment. Alternatively, the EMT pathway inhibitor may be used as a second line treatment after treatment with a platinum-based chemotherapeutic agent.

TABLE I
EMT pathway inhibitors

| DRUG NAME | COMPANY | INDICATION | PRODUCT STAGE |
|---|---|---|---|
| gilteritinib | Astellas | NSCLC | Ph III |
| fumarate | | Leukemia | Ph II |
| MGCD-265 | Mirati Therapeutics | Head & Neck, NSCLC | Ph II |
| MGCD-516 | Mirati Therapeutics | Lung | Ph I |
| S-49076 | Servier | NSCLC, Brain | Ph II |
| | | Colon, HCC | Pre-clinical |
| BPI-9016 | Zhejiang | Gastic, Liver, Lung | Ph I |
| | BetaPharma | | |
| CT-053 | EC Pharm Co., Ltd. | Brain | Ph I |
| | | Bladder, Breast, HCC, RCC, Lung, | Pre-clinical |
| | | Ovarian | |
| BGB-324 | BerGenBio | Leukemia, Lung | Ph I |
| | | Breast, Pancreatic | Pre-clinical |
| BGB-10C9 | BerGenBio | Pancreatic | Pre-clinical |
| Misc AXLi | BerGenBio | Oncology | Pre-clinical |
| HuMax-AXL- | GenMab | Solid Tumor | Pre-clinical |
| ADC | | | |
| LDC-2636 | Lead Discovery | Leukemia | Pre-clinical |
| | Center | | |
| Misc AXLi | Protelica | Oncology | Pre-clinical |
| | Incorporated | | |
| NPS-1034 | NeoPharm | NSCLC | Pre-clinical |
| Q-701 | Qurient Co., Ltd. | Oncology | Pre-clinical |
| RXDX-106 | Ignyta, Inc. | Leukemia, Breast, GI, Melanoma; | Pre-clinical |
| | | mCRC, NSCLC, Ovarian, Pancreatic | |
| SGI-7079 | Tolero | NSCLC | Pre-clinical |
| | Pharmaceuticals, | | |
| | Inc. | | |
| TP-0903 | Tolero | Leukemia; Head And Neck, Lung, | Pre-clinical |
| | Pharmaceuticals, | Pancreatic | |
| | Inc. | | |
| Misc AXLi | SignalChem | Oncology | Pre-clinical |
| | Lifesciences | | |
| Misc AXLi | University of | NSCLC | Pre-clinical |
| | Colorado | | |
| Misc AXLi | Kolltan | Oncology | Discovery |
| | Pharmaceuticals | | |

By “SRC pathway inhibitor” is meant a therapeutic agent, such as a pharmaceutical drug, that acts to inhibit signalling by the SRC pathway. The inhibitor may be specific for the SRC pathway. Thus, in certain embodiments the SRC pathway inhibitor is not a multi-pathway inhibitor. In further embodiments the SRC pathway inhibitor is a (specific) inhibitor of an SRC family kinase.

In certain embodiments the SRC pathway inhibitor is selected from Table J. In further embodiments the SRC pathway inhibitor is not Dasatinib and/or pazopanib (hydrochloride), which are multi-targeted pathway inhibitors.

TABLE J
SRC pathway inhibitors

| | | | PRODUCT |
|---|---|---|---|
| DRUG NAME | COMPANY | INDICATION | STAGE |
| Ilorasertib | AbbVie | Solid Tumor | Ph II |
| | | Ovarian | Pre-clinical |
| KX-01 | Athenex | Leukemia | PhI |
| pazopanib | GSK | mRCC | PhII |
| hydrochloride + | | | |
| pembrolizumab | | | |
| tesevatinib | Kadmon | mBrain, | PhII |
| tosylate | Corporation | mBreast, | |
| | | NSCLC | |
| VAL-201 | ValiRx Plc | Breast, | PhII |
| | | mProstate, | |
| | | Ovarian | |
| AZD-0424 | AZ | Oncology | PhI |
| BGB-102 | BeiGene(Beijing) | Oncology | PhI |
| KX-02 | Athenex, Inc. | Brain, | PhI |
| | | Lymphoma | |
| rebastinib tosylate | Deciphera | Leukemia | PhI |
| | Pharmaceuticals | mBreast | Pre-clinical |
| ASN-006 | Asana BioSciences | Oncology | Pre-clinical |
| CCT-196969 | Basilea | Melanoma | Pre-clinical |
| CCT-241161 | Pharmaceutica AG | | |
| ORB-0001 | OriBase Pharma | Leukemia | Pre-clinical |
| Misc | MI.TO. Technology | Oncology | Pre-clinical |
| | S.r.L. | | |
| Misc | University of | Prostate | Pre-clinical |
| | Toledo | | |
| RK-20449 | Riken Advanced | Leukemia | Pre-clinical |
| | Science Institute | | |

Various others in Discovery stage by Academic institutions

According to all aspects of the invention the platinum-based chemotherapeutic agent may comprise one or more of, or be selected from carboplatin, cisplatin, oxaliplatin, satraplatin, picoplatin, Nedaplatin, Triplatin and/or Lipoplatin.

According to all aspects of the invention the taxane may comprise Paclitaxel and/or Docetaxel. In specific embodiments the therapeutic agent is a taxane and the cancer is prostate cancer. The prostate cancer may be metastatic prostate cancer, in particular de novo metastatic prostate cancer.

The inhibitors described herein may act by inhibiting the expression (reducing the levels) and/or the function of one (or more) targets. Inhibition of function can include inhibiting interactions with one (or more) binding partners.

By “anti-angiogenic therapeutic agent” is meant a therapeutic agent, such as a pharmaceutical drug, that acts to inhibit angiogenesis. Examples of anti-angiogenic therapeutic agents include VEGF pathway-targeted therapeutic agents, including multi-targeted pathway inhibitors (VEGF/PDGF/FGF/EGFT/FLT-3/c-KIT), Angiopoietin-TIE2 pathway inhibitors, endogenous angiogenic inhibitors, and immunomodulatory Agents. VEGF specific inhibitors include, but are not limited to, Bevacizumab (Avastin), Afibercept (VEGF Trap), IMC-1121B (Ramucirumab). Multi-targeted pathway inhibitors include, but are not limited to, Imatinib (Gleevec), Sorafenib (Nexavar), Gefitinib (Iressa), Sunitinib (Sutent), Erlotinib, Tivozinib, Cediranib (Recentin), Pazopanib (Votrient), BIBF 1120 (Vargatef), Dovitinib, Semaxanib (Sugen), Axitinib (AG013736), Vandetanib (Zactima), Nilotinib (Tasigna), Dasatinib (Sprycel), Vatalanib, Motesanib, ABT-869, TKI-258. Angiopoietin-TIE2 pathway inhibitors include, but are not limited to, AMG-386 (Trebananib), PF-4856884 CVX-060, CEP-11981, CE-245677, MEDI-3617, CVX-241, Trastuzumab (Herceptin). Endogenous angiogenic inhibitors include, but are not limited to, Thombospondin, Endostatin, Tumstatin, Canstatin, Arrestin, Angiostatin, Vasostatin, Interferon alpha. Immunomodulatory Agents include, but are not limited to, Thalidomide and Lenalidomide. The inhibitor may be specific for angiogenesis processes or pathways. In certain embodiments the anti-angiogenic therapeutic agent is not a multi-pathway inhibitor.

In certain embodiments the anti-angiogenic therapeutic agent is selected from Table K.

TABLE K
Anti-angiogenic therapeutic agents

| Trade Name | Generic Name | Companies |
|---|---|---|
| Avastin | Bevacizumab | Chugai Pharmaceutical Co., |
| | | Ltd; Genentech, Inc.; PDL |
| | | BioPharma, Inc.; Roche |
| Nexavar | Sorafenib | Bayer AG; Onyx |
| | | Pharmaceuticals, Inc. |
| Nintedanib | Nintedanib | Boehringer Ingelheim GmbH |
| VEGFR-2 siRNA Formulated | | EGEN, Inc. |
| With Staramine-mPEG | | |
| Small Molecule Vegf Inhibitor | | Interprotein Corporation |
| TRC105 | | Santen Pharmaceutical Co., |
| | | Ltd; Tracon Pharmaceuticals |
| Zaltrap | Ziv-Aflibercept | Regeneron Pharmaceuticals, |
| | | Inc.; Sanofi |
| CS3158 | | Shenzhen Chipscreen |
| | | Biosciences |
| Fruquintinib | Fruquintinib | Eli Lilly & Co.; Hutchison |
| | | Medipharma Limited |
| Zactima | Vandetanib | AstraZeneca PLC |
| Ramucirumab | Ramucirumab | Dyax Corporation; Eli Lilly & |
| | | Co.; ImClone Systems |
| Dovitinib | Dovitinib | Novartis AG |
| hVEGF-trunc Vaccine | | Immunovo BV; Pepscan |
| Votrient | Pazopanib | GlaxoSmithKline plc |
| Inlyta | Axitinib | Pfizer, Inc.; Sfj |
| | | Pharmaceuticals, Inc. |
| Stivarga | Regorafenib | Bayer AG; Onyx |
| | | Pharmaceuticals, Inc. |
| Iclusig | Ponatinib | Ariad Pharmaceuticals, Inc.; |
| | | Specialised Therapeutics |
| | | Australia, Pty, Ltd. |
| Lucitanib | Lucitanib | Clovis Oncology Inc; Ethical |
| | | Oncology Science; Les |
| | | Laboratoires Servier; Shanghai |
| | | Institute of Materia Medica |
| E7080 | Lenvatinib | Eisai Co., Ltd.; Sfj |
| | | Pharmaceuticals, Inc. |
| Recentin | Cediranib | AstraZeneca PLC |
| Brivanib Alaninate | Brivanib Alaninate | Bristol-Myers Squibb |
| Cometriq | Cabozantinib | Bristol-Myers Squibb; Exelixis, |
| | | Inc.; Swedish Orphan Biovitrum |
| OTS102 | | Oncotherapy Science |
| Tivozanib | Tivozanib | AVEO Oncology; Astellas |
| | | Pharma, Inc.; Kirin |
| | | Pharmaceutical; Kyowa Hakko |
| | | Kirin Pharma, Inc |
| Sutent | Sunitinib Malate | Pfizer, Inc. |
| APX003 | | Apexigen, Inc.; Simcere |
| | | Pharmaceutical Group |
| Sareum VEGFR-3 Program | | Sareum Ltd. |
| PRS-050 | | Pieris AG |
| X-82 | | Xcovery, Inc. |
| CM-082 | | AnewPharma; Xcovery, Inc. |
| Pieris/Syngenta Anticalin | | Pieris AG; Syngenta |
| Program | | Corporation |
| CTx-0357927 | | Bionomics Ltd.; Cancer |
| | | Therapeutics Crc |
| ABT-869 | Linifanib | Abbott Laboratories; Abbvie; |
| | | Genentech, Inc.; Roche |
| MGCD265 | | Methylgene, Inc. |
| Dalantercept | | Acceleron Pharma |
| Norcantharidin | Norcantharidin | Shandong Hualu |
| | | Pharmaceutical Co., Ltd. |
| NOX-S93 | | Noxxon Pharma AG |
| VEGF Inhibitor Program | | Legochembiosciences, Inc. |
| R3 Antibody | | Affitech A/S; Peregrine |
| | | Pharmaceuticals, Inc. |
| AT001/r84 | | Affitech A/S; Peregrine |
| | | Pharmaceuticals, Inc. |
| Muparfostat | Muparfostat Sodium | Medigen Biotechnology Corp; |
| | | Progen Pharmaceuticals |
| | | Limited |
| Foretinib | Foretinib | Exelixis, Inc.; GlaxoSmithKline |
| | | plc |
| Telatinib | Telatinib | Act Biotech, Inc.; Bayer AG; |
| | | Eddingpharm (Cayman), Inc. |
| YN968D1 | Apatinib | Advenchen Laboratories, LLC |
| AL3818 | | Advenchen Laboratories, LLC |
| AL3810 | | Advenchen Laboratories, LLC |
| AL8326 | | Advenchen Laboratories, LLC |
| Icrucumab | Icrucumab | Eli Lilly & Co.; ImClone |
| | | Systems |
| PTC299 | | PTC Therapeutics, Inc. |
| Aplidin | Plitidepsin | PharmaMar |
| Veglin | Vascular Endothelial Growth | Vasgene Therapeutics |
| | Factor Antisense | |
| | Oligonucleotide | |
| BMS-817378 | | Bristol-Myers Squibb; Simcere |
| | | Pharmaceutical Group |
| MG516 | | Methylgene, Inc. |
| FP-1039 | | Five Prime Therapeutics, Inc.; |
| | | GlaxoSmithKline plc |
| IMC-3C5 | Vegfr 3 Monoclonal Antibody | Circadian Technologies |
| | | Limited; Eli Lilly & Co.; ImClone |
| | | Systems |
| TAS-115 | | Otsuka Pharmaceutical Co., |
| | | Ltd; Taiho Pharmaceutical Co., |
| | | Ltd |
| TRAP Based VEGFR2 Inhibitor | | Telik, Inc. |
| TLK60404 | | Telik, Inc. |
| Trap Based Ar + Vegf Dual | | Telik, Inc. |
| Inhibitor | | |
| RG7221 | | Roche |
| DCC-2701 | | Deciphera Pharmaceuticals |
| DP-2473 | | Deciphera Pharmaceuticals |
| DP-2514 | | Deciphera Pharmaceuticals; Eli |
| | | Lilly & Co. |
| VEGF R-2 Inhibitor | | Bristol-Myers Squibb |
| Erbb + Vegf Receptor Inhibitor | | Bristol-Myers Squibb |
| Motesanib | Motesanib Diphosphate | Amgen, Inc.; Millennium |
| | | Pharmaceuticals, Inc.; Takeda |
| | | Pharmaceutical Company |
| | | Limited |
| Semaxanib | Semaxanib | Pfizer, Inc. |
| VGX-200 | | Circadian Technologies |
| | | Limited; Teva Pharmaceutical |
| | | Industries Ltd |
| E7050 | Golvatinib | Eisai Co., Ltd. |
| GSK089 | | GlaxoSmithKline plc |
| KW-2401 | Irinotecan | Kyowa Hakko Kirin Pharma, |
| | | Inc |
| GNR-011 | Apagin | International Biotechnology |
| | | Center Generium |
| CYC116 | | Cyclacel Pharmaceuticals, Inc. |
| FAK-FLT3-VEGFR3 Program | | Cancer Therapeutics Crc |
| DMI-6867 | | Ampio Pharmaceuticals, Inc. |
| VGX-100 | | Circadian Technologies Limited |
| A6 | | Angstrom Pharmaceuticals, |
| | | Inc. |
| 1181 | | Egenix Inc |
| 4EGI-1 | | Egenix Inc |
| Egenix Cancer Therapeutics | | Egenix Inc |
| Program | | |
| CFAK-C4 | | Curefaktor Pharmaceuticals |
| PMX 2058 | | Pharminox Limited |
| GFB-204 | | H. Lee Moffitt Cancer Center & |
| | | Research Institute; Kirax |
| | | Corporation; Yale University |
| | | Office of Cooperative Research |

According to all aspects of the invention the method may further comprise obtaining a test sample from the subject. In certain embodiments the methods involving determining gene expression are in vitro methods performed on an isolated sample.

According to all aspects of the invention samples may be of any suitable form including any material, biological fluid, tissue, or cell obtained or otherwise derived from an individual. Typically, the sample includes cancer cells or genetic material (DNA or RNA) derived from the cancer cells, to include cell-free genetic material (e.g. found in the peripheral blood). In specific embodiments the sample comprises, consists essentially of or consists of a formalin-fixed paraffin-embedded biopsy sample. In further embodiments the sample comprises, consists essentially of or consists of a fresh/frozen (FF) sample. The sample may comprise, consist essentially of or consist of tumour (cancer) tissue, optionally ovarian tumour (cancer) tissue. The sample may comprise, consist essentially of or consist of tumour (cancer) cells, optionally ovarian tumour (cancer) cells. The tissue sample may be obtained by any suitable technique.

Examples include a biopsy procedure, optionally a fine needle aspirate biopsy procedure. Body fluid samples may also be utilised. Suitable sample types include blood (including whole blood, leukocytes, peripheral blood mononuclear cells, buffy coat, plasma, and serum), sputum, tears, mucus, nasal washes, nasal aspirate, breath, urine, semen, saliva, meningeal fluid, amniotic fluid, glandular fluid, lymph fluid, nipple aspirate, bronchial aspirate, synovial fluid, joint aspirate, ascites, cells, a cellular extract, and cerebrospinal fluid. This also includes experimentally separated fractions of all of the preceding. For example, a blood sample can be fractionated into serum or into fractions containing particular types of blood cells, such as red blood cells or white blood cells (leukocytes). If desired, a sample can be a combination of samples from an individual, such as a combination of a tissue and fluid sample. The term “sample” also includes materials containing homogenized solid material, such as from a stool sample, a tissue sample, or a tissue biopsy, for example. The term “sample” also includes materials derived from a tissue culture or a cell culture, including tissue resection and biopsy samples. Example methods for obtaining a sample include, e.g., phlebotomy, swab (e.g., buccal swab). Samples can also be collected, e.g., by micro dissection (e.g., laser capture micro dissection (LCM) or laser micro dissection (LMD)), bladder wash, smear (e.g., a PAP smear), or ductal lavage. A “sample” obtained or derived from an individual includes any such sample that has been processed in any suitable manner after being obtained from the individual. The methods of the invention as defined herein may begin with an obtained sample and thus do not necessarily incorporate the step of obtaining the sample from the patient. As used herein, the term “patient” includes human and non-human animals. The preferred patient for treatment is a human. “Patient,” “individual” and “subject” are used interchangeably herein.

According to all aspects of the invention the cancer may be ovarian, prostate, colon or lung cancer or melanoma. In certain embodiments the ovarian cancer is serous ovarian cancer. In specific embodiments the ovarian cancer is high grade serous ovarian cancer. In certain embodiments the lung cancer is non-small cell lung cancer and/or lung adenocarcinoma. The cancer may also be leukaemia, brain cancer, glioblastoma, head and neck cancer, liver cancer, stomach cancer, colorectal cancer, thyroid cancer, neuroendocrine cancer, gastrointestinal stromal tumors (GIST), gastric cancer, lymphoma, throat cancer, breast cancer, skin cancer, melanoma, multiple myeloma, sarcoma, cervical cancer, testicular cancer, bladder cancer, endocrine cancer, endometrial cancer, esophageal cancer, glioma (optionally lower grade glioma), lymphoma, neuroblastoma, osteosarcoma, pancreatic cancer, pituitary cancer, renal cancer (optionally renal clear cell cancer and/or renal papillary cancer), and the like. As used herein, colorectal cancer encompasses cancers that may involve cancer in tissues of both the rectum and other portions of the colon as well as cancers that may be individually classified as either colon cancer or rectal cancer. In certain embodiments the cancer is not prostate cancer and/or glioblastoma.

Optionally, according to all aspects, the method may comprise measuring the expression levels of at least around 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46 or each of the biomarkers listed in Table A and/or Table B. Combinations from Tables A and B are also envisaged. “Around” may mean plus or minus five. By “corresponding” may mean that the probe hybridizes to the gene/biomarker or can be used to detect expression of the gene/biomarker. Smaller gene signatures may be based around those markers having greater weight values in Tables A and B and thus, in some embodiments, sub-signatures are generated by taking a selection of the larger signatures in numerical order. Thus, for example, a 5 gene signature may be composed of the first 5 genes listed in Table A and/or Table B. It could also be composed of the 5 genes with the highest weight values from Tables A and B combined. In other embodiments, the gene signatures may comprise one of the markers with the highest weight values (e.g. selected from the top 2, 3, 4, 5, 6, 7, 8, 9, or 10 markers), either alone or combined with other markers. In other embodiments the methods may comprise measuring the expression levels of one or more up to all of the following biomarkers: GJB2, CDH11, GFPT2, COL10A1, ANGPTL2, THBS1, RAB31, THBS2, INHBA, MMP14, VCAN, PLAU, FAP, FN1.

Optionally, according to all aspects, the methods may comprise measuring the expression levels of one or more up to all of the following biomarkers: TMEM200A, GJB2, MMP13, GFPT2, POSTN, BICC1, MRVI1, COL11A1, IGFL2, NTM, BGN, COL10A1, RAB31, ANGPTL2, PLAU, COL8A1, MIR1245, POLD2, NKD2, FZD1, COPZ2, ITGA5, VGLL3, INHBA, MMP14, THBS2, RUNX2, TIMP3, SFRP2, COL1A2, COL5A2, SERPINF1, KIF26B, ALPK2, CTSK, LOXL1 and FAP (optionally together with one or more up to all of the following biomarkers: CDH11, PMP22, LUM, COL3A1, VCAN, TNFAIP6, MMP2 and FN1); and/or one or more up to all of the following biomarkers: GJB2, GFPT2, COL10A1, ANGPTL2, THBS1, RAB31, THBS2, INHBA, MMP14, PLAU and FAP (optionally together with one or more up to all of the following biomarkers: CDH11, VCAN, COL5A1 and FN1).

Optionally, according to all aspects, the methods may comprise measuring the expression levels of one or more up to all of the following biomarkers: TMEM200A, GJB2, MMP13, GFPT2, POSTN, BICC1, CDH11, MRVI1, PMP22, COL11A1, IGFL2, LUM, NTM, BGN, COL3A1, COL10A1, RAB31, ANGPTL2, PLAU, COL8A1, MIR1245, POLD2, NKD2, FZD1, COPZ2, ITGA5, VGLL3, INHBA, MMP14, VCAN, THBS2, RUNX2, TIMP3, SFRP2, COL1A2, COL5A2, SERPINF1, KIF26B, TNFAIP6, ALPK2, CTSK, LOXL1 and FAP (optionally together with one or more up to all of the following biomarkers: MMP2 and FN1); and/or one or more up to all of the following biomarkers: GJB2, CDH11, GFPT2, COL10A1, ANGPTL2, THBS1, RAB31, THBS2, INHBA, MMP14, VCAN, PLAU, COL5A1 and FAP (optionally together with FN1).

In further embodiments the methods may comprise measuring the expression levels of one or more, up to all of the biomarkers in Table 13 with an LCI C-index of more than 0.5. In yet further embodiments the methods may comprise measuring the expression levels of one or more, up to all of the top 10 ranked biomarkers in Table 14 and/or Table 15. In specific embodiments the methods may comprise measuring the expression levels of the sets of 22, 19, 17, 13, 11, 9, 8, 7, 6 and 5 biomarkers listed below. Each of these signatures has been shown to give high levels of performance in identifying the relevant molecular subgroup of cancer:

| 22 gene | | 19 gene | | 17 gene | | 13 gene | | 11 gene | |
|---|---|---|---|---|---|---|---|---|---|
| Entrez | | Entrez | | Entrez | | Entrez | | Entrez | |
| Gene | Gene | Gene | Gene | Gene | Gene | Gene | Gene | Gene | Gene |
| ID | Name | ID | Name | ID | Name | ID | Name | ID | Name |
| 1009 | CDH11 | 1009 | CDH11 | 1009 | CDH11 | 1009 | CDH11 | 1009 | CDH11 |
| 11031 | RAB31 | 11031 | RAB31 | 11031 | RAB31 | 11031 | RAB31 | 1289 | COL5A1 |
| 1278 | COL1A2 | 1289 | COL5A1 | 1289 | COL5A1 | 1289 | COL5A1 | 1300 | COL10A1 |
| 1281 | COL3A1 | 1300 | COL10A1 | 1300 | COL10A1 | 1300 | COL10A1 | 1462 | VCAN |
| 1289 | COL5A1 | 1462 | VCAN | 1462 | VCAN | 1462 | VCAN | 2191 | FAP |
| 1300 | COL10A1 | 2191 | FAP | 2191 | FAP | 2191 | FAP | 2706 | GJB2 |
| 1462 | VCAN | 2200 | FBN1 | 2200 | FBN1 | 2706 | GJB2 | 4323 | MMP14 |
| 2191 | FAP | 2335 | FN1 | 2335 | FN1 | 3624 | INHBA | 5328 | PLAU |
| 2200 | FBN1 | 23452 | ANGPTL2 | 23452 | ANGPTL2 | 4323 | MMP14 | 7057 | THBS1 |
| 2335 | FN1 | 2706 | GJB2 | 2706 | GJB2 | 5328 | PLAU | 7058 | THBS2 |
| 23452 | ANGPTL2 | 3624 | INHBA | 3624 | INHBA | 7057 | THBS1 | 9945 | GFPT2 |
| 2706 | GJB2 | 4060 | LUM | 4060 | LUM | 7058 | THBS2 | | |
| 3624 | INHBA | 4323 | MMP14 | 4323 | MMP14 | 9945 | GFPT2 | | |
| 3678 | ITGA5 | 5328 | PLAU | 5328 | PLAU | | | | |
| 4060 | LUM | 633 | BGN | 7057 | THBS1 | | | | |
| 4323 | MMP14 | 7057 | THBS1 | 7058 | THBS2 | | | | |
| 5328 | PLAU | 7058 | THBS2 | 9945 | GFPT2 | | | | |
| 633 | BGN | 9509 | ADAMTS2 | | | | | | |
| 7057 | THBS1 | 9945 | GFPT2 | | | | | | |
| 7058 | THBS2 | | | | | | | | |
| 9509 | ADAMTS2 | | | | | | | | |
| 9945 | GFPT2 | | | | | | | | |

| 9 gene | | 8 gene | | 7 gene | | 6 gene | | 5 gene | |
|---|---|---|---|---|---|---|---|---|---|
| Entrez | | Entrez | | Entrez | | Entrez | | Entrez | |
| Gene | Gene | Gene | Gene | Gene | Gene | Gene | Gene | Gene | Gene |
| ID | Name | ID | Name | ID | Name | ID | Name | ID | Name |
| 1009 | CDH11 | 1289 | COL5A1 | 1462 | VCAN | 1462 | VCAN | 1462 | VCAN |
| 1289 | COL5A1 | 1462 | VCAN | 2191 | FAP | 2191 | FAP | 2191 | FAP |
| 1462 | VCAN | 2191 | FAP | 2706 | GJB2 | 2706 | GJB2 | 2706 | GJB2 |
| 2191 | FAP | 2706 | GJB2 | 5328 | PLAU | 7057 | THBS1 | 7057 | THBS1 |
| 2706 | GJB2 | 5328 | PLAU | 7057 | THBS1 | 7058 | THBS2 | 7058 | THBS2 |
| 5328 | PLAU | 7057 | THBS1 | 7058 | THBS2 | 9945 | GFPT2 | | |
| 7057 | THBS1 | 7058 | THBS2 | 9945 | GFPT2 | | | | |
| 7058 | THBS2 | 9945 | GFPT2 | | | | | | |
| 9945 | GFPT2 | | | | | | | | |

Combinations of these signatures are also envisaged, for example to generate suitable 2, 3, 4, 10, 12, 14, 16, 18, 20 and 21 gene signatures. Thus, for example, a 10 gene signature may be formed by adding a single gene to the 9 gene signature. This gene could be selected from the additional genes included in another signature, for example in the 11 gene signature. Alternatively it could be derived from elsewhere and tested according to the methods known in the art and described herein.

The expression levels of the biomarkers in these sets may be measured using the probesets listed in Tables E, F and L as appropriate for each biomarker.

TABLE L
Probeset information for FBN1 and ADAMTS2

| | | | No. | | |
|---|---|---|---|---|---|
| | | | probes | | Gene |
| Probe Set ID | Type | Orientation | aligned | Ensembl Gene | Symbol |
| OC3P.7887.C1_s_at | Expression | Sense | 11 | ENSG00000166147 | FBN1 |
| | probeset | (Fully | | | |
| | | Exonic) | | | |
| OC3SNG.6052- | Expression | Sense | 11 | ENSG00000166147 | FBN1 |
| 16a_s_at | probeset | (Fully | | | |
| | | Exonic) | | | |
| OC3SNGn.8707- | Expression | Sense | 11 | ENSG00000166147 | FBN1 |
| 2674a_s_at | probeset | (Fully | | | |
| | | Exonic) | | | |
| OC3SNGnh.5433_at | Expression | Sense | 9 | ENSG00000166147 | FBN1 |
| | probeset | (includes | | | |
| | | Intronic) | | | |
| OCADA.4751_s_at | Expression | Sense | 11 | ENSG00000166147 | FBN1 |
| | probeset | (Fully | | | |
| | | Exonic) | | | |
| OCADNP.2122_s_at | Expression | Sense | 9 | ENSG00000166147 | FBN1 |
| | probeset | (Fully | | | |
| | | Exonic) | | | |
| OCMX.14880.C1_s_at | Expression | Sense | 10 | ENSG00000166147 | FBN1 |
| | probeset | (Fully | | | |
| | | Exonic) | | | |
| OC3SNGn.1835- | Expression | Sense | 11 | ENSG00000087116 | ADAMTS2 |
| 5a_s_at | probeset | (Fully | | | |
| | | Exonic) | | | |
| OCADA.5272_s_at | Expression | Sense | 7 | ENSG00000087116 | ADAMTS2 |
| | probeset | (Fully | | | |
| | | Exonic) | | | |
| OCADNP.3907_s_at | Expression | Sense | 11 | ENSG00000087116 | ADAMTS2 |
| | probeset | (Fully | | | |
| | | Exonic) | | | |
| OCHPRC.106_s_at | Expression | Sense | 11 | ENSG00000087116 | ADAMTS2 |
| | probeset | (Fully | | | |
| | | Exonic) | | | |

| | Entrez | | | | SEQ |
|---|---|---|---|---|---|
| | Gene | | | | ID |
| Probe Set ID | ID | Description | Chromosome | Strand | NO. |
| OC3P.7887.C1_s_at | 2200 | fibrillin 1 | Chr 15 | Reverse | 292 |
| | | [Source: HGNC | | Strand | |
| | | Symbol; Acc: 3603] | | | |
| OC3SNG.6052- | 2200 | fibrillin 1 | Chr 15 | Reverse | 293 |
| 16a_s_at | | [Source: HGNC | | Strand | |
| | | Symbol; Acc: 3603] | | | |
| OC3SNGn.8707- | 2200 | fibrillin 1 | Chr 15 | Reverse | 294 |
| 2674a_s_at | | [Source: HGNC | | Strand | |
| | | Symbol; Acc: 3603] | | | |
| OC3SNGnh.5433_at | 2200 | fibrillin 1 | Chr 15 | Reverse | 295 |
| | | [Source: HGNC | | Strand | |
| | | Symbol; Acc: 3603] | | | |
| OCADA.4751_s_at | 2200 | fibrillin 1 | Chr 15 | Reverse | 296 |
| | | [Source: HGNC | | Strand | |
| | | Symbol; Acc: 3603] | | | |
| OCADNP.2122_s_at | 2200 | fibrillin 1 | Chr 15 | Reverse | 297 |
| | | [Source: HGNC | | Strand | |
| | | Symbol; Acc: 3603] | | | |
| OCMX.14880.C1_s_at | 2200 | fibrillin 1 | Chr 15 | Reverse | 298 |
| | | [Source: HGNC | | Strand | |
| | | Symbol; Acc: 3603] | | | |
| OC3SNGn.1835- | 9509 | ADAM | Chr 5 | Reverse | 299 |
| 5a_s_at | | metallopeptidase | | Strand | |
| | | with | | | |
| | | thrombospondin | | | |
| | | type 1 motif, 2 | | | |
| | | [Source: HGNC | | | |
| | | Symbol; Acc: 218] | | | |
| OCADA.5272_s_at | 9509 | ADAM | Chr 5 | Reverse | 300 |
| | | metallopeptidase | | Strand | |
| | | with | | | |
| | | thrombospondin | | | |
| | | type 1 motif, 2 | | | |
| | | [Source: HGNC | | | |
| | | Symbol; Acc: 218] | | | |
| OCADNP.3907_s_at | 9509 | ADAM | Chr 5 | Reverse | 301 |
| | | metallopeptidase | | Strand | |
| | | with | | | |
| | | thrombospondin | | | |
| | | type 1 motif, 2 | | | |
| | | [Source: HGNC | | | |
| | | Symbol; Acc: 218] | | | |
| OCHPRC.106_s_at | 9509 | ADAM | Chr 5 | Reverse | 302 |
| | | metallopeptidase | | Strand | |
| | | with | | | |
| | | thrombospondin | | | |
| | | type 1 motif, 2 | | | |
| | | [Source: HGNC | | | |
| | | Symbol; Acc: 218] | | | |

In particular embodiments the at least 1 biomarker selected from Table B is not COL5A1. In certain embodiments the at least 1 biomarker selected from Table A or Table B is not one or more up to all of ANGPTL2, CDH11, COL1A2, COL8A1, LOXL1, MMP14, POLD2 and/or TIMP3. Additionally or alternatively, in certain embodiments the at least 1 biomarker selected from Table A or Table B is not one or more up to all of CDH11, PMP22, LUM, COL3A1, VCAN, TNFAIP6, MMP2, FN1 and/or COL5A1. In further embodiments the at least 1 biomarker selected from Table A or Table B is not MMP2 and/or FN1. In specific embodiments the at least 1 biomarker selected from Table A or Table B does not consist of from 1 to 63 of the biomarkers shown in Table M. In further specific embodiments the EMT pathway inhibitor is ALM201 and the at least 1 biomarker selected from Table A or Table B does not consist of from 1 to 63 of the biomarkers shown in Table M.

TABLE M
GeneSymbol

IGF2
SOX11
INS
CXCL17
SLC5A1
TMEM45A
CXCR2P1
MFAP2
MATN3
RTP4
COL3A1
CDR1
RARRES3
TNFSF10
NUAK1
SNORD114-14
SRPX
SPARC
GJB1
TIMP3
ISLR
TUBA1A
DEXI
BASP1
PXDN
GBP4
SLC28A3
HLA-DRA
TAP2
ACSL5
CDH11
PSMB9
MMP14
CD74
LOXL1
CIITA
ZNF697
SH3RF2
MIR198
COL1A2
TNFRSF14
COL8A1
C21orf63
TAP1
PDPN
RHOBTB3
BCL11A
HLA-DOB
XAF1
ARHGAP26
POLD2
DPYSL2
COL4A1
ID3
CFB
NID1
FKBP7
TIMP2
RCBTB1
ANGPTL2
ENTPD7
SHISA4
HINT1

On the basis of the information provided herein other biomarker signatures may be derived by the skilled person for use according to the invention. By using one or more of the biomarker signatures described herein (such as the 15 or 45 gene signature) the skilled person could classify a sample set into those positive and negative for the biomarker signature. The skilled person could then derive further signatures using methods described herein or known in the art (such as partial least squares paired with forward feature selection) that reproduce the classification ability of the biomarker signatures described herein. Alternatively, the skilled person could carry out the gene expression profiling and hierarchical clustering described herein and in WO2012/167278 to identify samples that fall within the EMT/Angio-Immune/MAPK pathway molecular subgroup of cancer identified by the present inventors. The skilled person could then use methods such as partial least squares paired with forward feature selection to derive further signatures that are able to detect the EMT/Angio-Immune/MAPK pathway molecular subgroup of cancer. The further signatures could be generated on an initial training dataset and then tested in a subsequent dataset for their ability to identify the EMT/Angio-Immune/MAPK pathway molecular subgroup of cancer or their classification ability.

Methods for determining the expression levels of the biomarkers are described in greater detail herein. Typically, the methods may involve contacting a sample obtained from a subject with a detection agent, such as primers/probes/antibodies (as discussed in detail herein) specific for the biomarker and detecting biomarker expression products.

According to all aspects of the invention the expression level of the gene or genes may be measured by any suitable method. Genes may also be referred to, interchangeably, as biomarkers. In certain embodiments the expression level is determined at the level of protein, RNA or epigenetic modification. The epigenetic modification may be DNA methylation.

The expression level may be determined by immunohistochemistry. By “Immunohistochemistry” is meant the detection of proteins in cells of a tissue sample by using a binding reagent such as an antibody or aptamer that binds specifically to the proteins. Accordingly, in a further aspect, the present invention relates to an antibody or aptamer that binds specifically to a protein product of at least one of the biomarkers described herein.

Antibodies useful for therapeutic and detection purposes as required herein may be of monoclonal or polyclonal origin. Fragments and derivative antibodies may also be utilised, to include without limitation Fab fragments, ScFv, single domain antibodies, nanoantibodies, heavy chain antibodies, aptamers, highly constrained bicyclic peptides (“bicycles”) etc. which retain specific binding function and these are included in the definition of “antibody”. Such antibodies are useful in the methods of the invention. Therapeutic antibodies may be conjugated to a drug to form an antibody drug conjugate. Many such ADC systems are known in the art. They may be used to measure the level of a particular protein, or in some instances one or more specific isoforms of a protein. The skilled person is well able to identify epitopes that permit specific isoforms to be discriminated from one another.

Methods for generating specific antibodies are known to those skilled in the art. Antibodies may be of human or non-human origin (e.g. rodent, such as rat or mouse) and be humanized etc. according to known techniques (Jones et al., Nature (1986) May 29-June 4; 321(6069):522-5; Roguska et al., Protein Engineering, 1996, 9(10):895-904; and Studnicka et al., Humanizing Mouse Antibody Frameworks While Preserving 3-D Structure. Protein Engineering, 1994, Vol. 7, pg 805).

In certain embodiments the expression level is determined using an antibody or aptamer conjugated to a label. By label is meant a component that permits detection, directly or indirectly. For example, the label may be an enzyme, optionally a peroxidase, or a fluorophore.

Where the antibody is conjugated to an enzyme a chemical composition may be used such that the enzyme catalyses a chemical reaction to produce a detectable product. The products of reactions catalyzed by appropriate enzymes can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers. In certain embodiments a secondary antibody is used and the expression level is then determined using an unlabeled primary antibody that binds to the target protein and a secondary antibody conjugated to a label, wherein the secondary antibody binds to the primary antibody.

Additional techniques for determining expression level at the level of protein include, for example, Western blot, immunoprecipitation, immunocytochemistry, mass spectrometry, ELISA and others (see ImmunoAssay: A Practical Guide, edited by Brian Law, published by Taylor & Francis, Ltd., 2005 edition). To improve specificity and sensitivity of an assay method based on immunoreactivity, monoclonal antibodies are often used because of their specific epitope recognition. Polyclonal antibodies have also been successfully used in various immunoassays because of their increased affinity for the target as compared to monoclonal antibodies.

Measuring mRNA in a biological sample may be used as a surrogate for detection of the level of the corresponding protein in the biological sample. Thus, the expression level of any of the genes described herein can also be detected by detecting the appropriate RNA. RNA from the sample may be converted into cDNA and the amount of the appropriate cDNA measured using any suitable method, for example via hybridization of (fluorescently labelled) probes. The amount of cDNA from the sample may then be compared with a reference amount of the relevant cDNA. cDNA based measurements may employ second generation sequencing technologies such as Illumina and Ion Torrent sequencing. Direct RNA measurements are also possible, for example using third generation sequencing technologies such as SMRT sequencing (Pacific Biosciences), nanopore sequencing and SeqLL (Helicos) sequencing.

Accordingly, in specific embodiments the expression level is determined by microarray, northern blotting, RNA-seq (RNA sequencing), in situ RNA detection or nucleic acid amplification. Nucleic acid amplification includes PCR and all variants thereof such as real-time and end point methods and qPCR. Other nucleic acid amplification techniques are well known in the art, and include methods such as NASBA, 3SR and Transcription Mediated Amplification (TMA). Other suitable amplification methods include the ligase chain reaction (LCR), selective amplification of target polynucleotide sequences (U.S. Pat. No. 6,410,276), consensus sequence primed polymerase chain reaction (U.S. Pat. No. 4,437,975), arbitrarily primed polymerase chain reaction (WO 90/06995), invader technology, strand displacement technology, and nick displacement amplification (WO 2004/067726). This list is not intended to be exhaustive; any nucleic acid amplification technique may be used provided the appropriate nucleic acid product is specifically amplified. Design of suitable primers and/or probes is within the capability of one skilled in the art. Various primer design tools are freely available to assist in this process such as the NCBI Primer-BLAST tool. Primers and/or probes may be at least 15, 16, 17, 18, 19, 20, 21, 22, 23, 24 or 25 (or more) nucleotides in length. mRNA expression levels may be measured by reverse transcription quantitative polymerase chain reaction (RT-PCR followed with qPCR). RT-PCR is used to create a cDNA from the mRNA. The cDNA may be used in a qPCR assay to produce fluorescence as the DNA amplification process progresses. By comparison to a standard curve, qPCR can produce an absolute measurement such as number of copies of mRNA per cell. Northern blots, microarrays, Invader assays, and RT-PCR combined with capillary electrophoresis have all been used to measure expression levels of mRNA in a sample. See Gene Expression Profiling: Methods and Protocols, Richard A. Shimkets, editor, Humana Press, 2004.

RNA-seq uses next-generation sequencing to measure changes in gene expression. RNA may be converted into cDNA or directly sequenced. Next generation sequencing techniques include pyrosequencing, SOLiD sequencing, Ion Torrent semiconductor sequencing, Illumina dye sequencing, single-molecule real-time sequencing or DNA nanoball sequencing.

In situ RNA detection involves detecting RNA without extraction from tissues and cells. In situ RNA detection includes In situ hybridization (ISH) which uses a labeled (e.g. radio labelled, antigen labelled or fluorescence labelled) probe (complementary DNA or RNA strand) to localize a specific RNA sequence in a portion or section of tissue, or in the entire tissue (whole mount ISH), or in cells. A branched DNA assay can also be used for RNA in situ hybridization assays with single molecule sensitivity. This approach includes ViewRNA assays.

Thus, in a further aspect the present invention relates to a kit comprising one or more oligonucleotide probes specific for an RNA product of at least 1 biomarker from Table A or Table B.

RNA expression may be determined by hybridization of RNA to a set of probes. The probes may be arranged in an array. Microarray platforms include those manufactured by companies such as Affymetrix, Illumina and Agilent. Examples of microarray platforms manufactured by Affymetrix include the U133 Plus2 array, the Almac proprietary Xcel™ array and the Almac proprietary Cancer DSAs®. In specific embodiments a sample of target nucleic acids is first prepared from the initial nucleic acid sample being assayed, where preparation may include labeling of the target nucleic acids with a label, e.g., a member of a signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the biomarkers whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acids provides information regarding expression for each of the biomarkers that have been probed, where the expression information is in terms of whether or not the gene is expressed and, typically, at what level, where the expression data, i.e., expression profile, may be both qualitative and quantitative.

In certain embodiments, measuring the expression levels of the at least 1 biomarker selected from Table A or Table B comprises contacting the sample with a set of nucleic acid probes or primers that bind to the at least 1 biomarker and detecting binding of the set of nucleic acid probes or primers to the at least 1 biomarker(s) by microarray, northern blotting, or nucleic acid amplification.

The methods described herein may further comprise extracting total nucleic acid or RNA from the sample. Suitable methods are known in the art and include use of commercially available kits such as RNeasy and GeneJET RNA purification kit.

In specific embodiments, expression of the at least one gene may be determined using one or more probes described herein.

These probes may also be incorporated into the kits of the invention. The probe sequences may also be used in order to design primers for detection of expression, for example by RT-PCR. Such primers may also be included in the kits of the invention.

The invention also relates to a system or device for performing a method as described herein.

Thus, the present invention relates to a system or test kit for selecting a treatment for a subject having a cancer, comprising:
(a) one or more testing devices for determining the expression level of at least 1 biomarker selected from Table A or Table B in a sample from the subject
(b) a processor; and
(c) storage medium comprising a computer application that, when executed by the processor, is configured to:
(i) access and/or calculate the determined expression levels of the at least 1 biomarker in the sample on the one or more testing devices
(ii) calculate from the expression level(s) of the at least 1 biomarker whether the sample from the subject is positive or negative for a biomarker signature comprising the at least 1 biomarker; and
(iii) output from the processor the selected treatment.

In certain embodiments:
(a) if the sample is positive for the biomarker signature a MAPK pathway inhibitor is selected and/or if the sample is negative for the biomarker signature a MAPK pathway inhibitor is not selected; and/or
(b) if the sample is positive for the biomarker signature an EMT pathway inhibitor is selected and/or if the sample is negative for the biomarker signature an EMT pathway inhibitor is not selected; and/or
(c) if the sample is positive for the biomarker signature an SRC pathway inhibitor is not selected and/or if the sample is negative for the biomarker signature an SRC pathway inhibitor is selected
(d) if the sample is positive for the biomarker signature an anti-angiogenic therapeutic agent is selected and/or if the sample is negative for the biomarker signature an anti-angiogenic therapeutic agent is not selected; and/or
(e) if the sample is positive for the biomarker signature a taxane is selected and/or if the sample is negative for the biomarker signature a taxane is not selected.

In yet a further aspect, the present invention relates to system or test kit for predicting the responsiveness of a subject with cancer to a therapeutic agent comprising:
(a) one or more testing devices for determining the expression level of at least 1 biomarker selected from Table A or Table B in a sample from the subject
(b) a processor; and
(c) storage medium comprising a computer application that, when executed by the processor, is configured to:
(i) access and/or calculate the determined expression levels of the at least 1 biomarker in the sample on the one or more testing devices
(ii) calculate from the expression level(s) of the at least 1 biomarker whether the sample from the subject is positive or negative for a biomarker signature comprising the at least 1 biomarker; and
(iii) output from the processor the predicted responsiveness.

In certain embodiments the subject is classified as
(a) predicted to be responsive to a MAPK pathway inhibitor if the sample is positive for the biomarker signature and/or predicted to be non-responsive to the MAPK pathway inhibitor if the sample is negative for the biomarker signature; and/or
(b) predicted to be responsive to an EMT pathway inhibitor if the sample is positive for the biomarker signature and/or predicted to be non-responsive to the EMT pathway inhibitor if the sample is negative for the biomarker signature; and/or
(c) predicted to be non-responsive to a SRC pathway inhibitor if the sample is positive for the biomarker signature and/or predicted to be responsive to the SRC inhibitor if the sample is negative for the biomarker signature; and/or
(d) predicted to be non-responsive to a platinum-based chemotherapeutic agent if the sample is positive for the biomarker signature and/or predicted to be responsive to a platinum-based chemotherapeutic agent if the sample is negative for the biomarker signature; and/or
(e) predicted to be responsive to an anti-angiogenic therapeutic agent if the sample is positive for the biomarker signature and/or predicted to be non-responsive to the anti-angiogenic therapeutic agent if the sample is negative for the biomarker signature; and/or
(f) predicted to be responsive to a taxane if the sample is positive for the biomarker signature and/or predicted to be non-responsive to the taxane if the sample is negative for the biomarker signature.

The invention also relates to a system or test kit for determining the clinical prognosis of a subject with cancer comprising:
a) one or more testing devices for determining the expression level of at least 1 biomarker selected from Table A or Table B in a sample from the subject
b) a processor; and
c) storage medium comprising a computer application that, when executed by the processor, is configured to:
(i) access and/or calculate the determined expression levels of the at least 1 biomarker in the sample on the one or more testing devices
(ii) calculate from the expression level(s) of the at least 1 biomarker whether the sample from the subject is positive or negative for a biomarker signature comprising the at least 1 biomarker; and
(iii) output from the processor the prognosis for the subject.

In certain embodiments the subject is classified as having a poor prognosis if the sample is positive for the biomarker signature and/or having a good prognosis if the sample is negative for the biomarker signature.

The system or test kit may further comprise a display for the output from the processor.

By testing device is meant a combination of components that allows the expression level of a gene to be determined. The components may include any of those described above with respect to the methods for determining expression level at the level of protein, RNA or epigenetic modification. For example the components may be antibodies, primers, detection agents and so on. Components may also include one or more of the following: microscopes, microscope slides, x-ray film, radioactivity counters, scintillation counters, spectrophotometers, colorimeters, fluorometers, luminometers, and densitometers.

The invention also relates to a computer application or storage medium comprising a computer application as defined above.

In certain example embodiments, provided is a computer-implemented method, system, and a computer program product for selecting a treatment for a subject having a cancer and/or prediction of the responsiveness of a subject with cancer to a therapeutic agent and/or determining the clinical prognosis of a subject with cancer, in accordance with the methods described herein. For example, the computer program product may comprise a non-transitory computer-readable storage device having computer-readable program instructions embodied thereon that cause the computer to:
(i) access and/or calculate the determined expression levels of the at least 1 biomarker selected from Table A or Table B in a sample on one or more testing devices;
(ii) calculate from the expression level(s) of the at least 1 biomarker whether the sample from the subject is positive or negative for a biomarker signature comprising the at least 1 biomarker in the sample; and,
(iii) provide an output.

In certain example embodiments, the computer-implemented method, system, and computer program product may be embodied in a computer application, for example, that operates and executes on a computing machine and a module. When executed, the application may select whether to administer a treatment to a subject having a cancer and/or predict the responsiveness of a subject with cancer to a therapeutic agent and/or determine the clinical prognosis of a subject with cancer, in accordance with the example embodiments described herein.

As used herein, the computing machine may correspond to any computers, servers, embedded systems, or computing systems. The module may comprise one or more hardware or software elements configured to facilitate the computing machine in performing the various methods and processing functions presented herein. The computing machine may include various internal or attached components such as a processor, system bus, system memory, storage media, input/output interface, and a network interface for communicating with a network, for example.

The computing machine may be implemented as a conventional computer system, an embedded controller, a laptop, a server, a customized machine, any other hardware platform, such as a laboratory computer or device, for example, or any combination thereof. The computing machine may be a distributed system configured to function using multiple computing machines interconnected via a data network or bus system, for example.

The processor may be configured to execute code or instructions to perform the operations and functionality described herein, manage request flow and address mappings, and to perform calculations and generate commands. The processor may be configured to monitor and control the operation of the components in the computing machine. The processor may be a general purpose processor, a processor core, a multiprocessor, a reconfigurable processor, a microcontroller, a digital signal processor (“DSP”), an application specific integrated circuit (“ASIC”), a graphics processing unit (“GPU”), a field programmable gate array (“FPGA”), a programmable logic device (“PLD”), a controller, a state machine, gated logic, discrete hardware components, any other processing unit, or any combination or multiplicity thereof. The processor may be a single processing unit, multiple processing units, a single processing core, multiple processing cores, special purpose processing cores, co-processors, or any combination thereof. According to certain example embodiments, the processor, along with other components of the computing machine, may be a virtualized computing machine executing within one or more other computing machines.

The system memory may include non-volatile memories such as read-only memory (“ROM”), programmable read-only memory (“PROM”), erasable programmable read-only memory (“EPROM”), flash memory, or any other device capable of storing program instructions or data with or without applied power. The system memory may also include volatile memories such as random access memory (“RAM”), static random access memory (“SRAM”), dynamic random access memory (“DRAM”), and synchronous dynamic random access memory (“SDRAM”). Other types of RAM also may be used to implement the system memory. The system memory may be implemented using a single memory module or multiple memory modules. While the system memory may be part of the computing machine, one skilled in the art will recognize that the system memory may be separate from the computing machine without departing from the scope of the subject technology. It should also be appreciated that the system memory may include, or operate in conjunction with, a non-volatile storage device such as the storage media.

The storage media may include a hard disk, a floppy disk, a compact disc read only memory (“CD-ROM”), a digital versatile disc (“DVD”), a Blu-ray disc, a magnetic tape, a flash memory, other non-volatile memory device, a solid state drive (“SSD”), any magnetic storage device, any optical storage device, any electrical storage device, any semiconductor storage device, any physical-based storage device, any other data storage device, or any combination or multiplicity thereof. The storage media may store one or more operating systems, application programs and program modules such as module, data, or any other information. The storage media may be part of, or connected to, the computing machine. The storage media may also be part of one or more other computing machines that are in communication with the computing machine, such as servers, database servers, cloud storage, network attached storage, and so forth.

The module may comprise one or more hardware or software elements configured to facilitate the computing machine with performing the various methods and processing functions presented herein. The module may include one or more sequences of instructions stored as software or firmware in association with the system memory, the storage media, or both. The storage media may therefore represent examples of machine or computer readable media on which instructions or code may be stored for execution by the processor. Machine or computer readable media may generally refer to any medium or media used to provide instructions to the processor. Such machine or computer readable media associated with the module may comprise a computer software product. It should be appreciated that a computer software product comprising the module may also be associated with one or more processes or methods for delivering the module to the computing machine via a network, any signal-bearing medium, or any other communication or delivery technology. The module may also comprise hardware circuits or information for configuring hardware circuits such as microcode or configuration information for an FPGA or other PLD.

The input/output (“I/O”) interface may be configured to couple to one or more external devices, to receive data from the one or more external devices, and to send data to the one or more external devices. Such external devices along with the various internal devices may also be known as peripheral devices. The I/O interface may include both electrical and physical connections for operably coupling the various peripheral devices to the computing machine or the processor. The I/O interface may be configured to communicate data, addresses, and control signals between the peripheral devices, the computing machine, or the processor. The I/O interface may be configured to implement any standard interface, such as small computer system interface (“SCSI”), serial-attached SCSI (“SAS”), fiber channel, peripheral component interconnect (“PCI”), PCI express (PCIe), serial bus, parallel bus, advanced technology attached (“ATA”), serial ATA (“SATA”), universal serial bus (“USB”), Thunderbolt, FireWire, various video buses, and the like. The I/O interface may be configured to implement only one interface or bus technology.

Alternatively, the I/O interface may be configured to implement multiple interfaces or bus technologies. The I/O interface may be configured as part of, all of, or to operate in conjunction with, the system bus. The I/O interface may include one or more buffers for buffering transmissions between one or more external devices, internal devices, the computing machine, or the processor.

The I/O interface may couple the computing machine to various input devices including mice, touch-screens, scanners, electronic digitizers, sensors, receivers, touchpads, trackballs, cameras, microphones, keyboards, any other pointing devices, or any combinations thereof. The I/O interface may couple the computing machine to various output devices including video displays, speakers, printers, projectors, tactile feedback devices, automation control, robotic components, actuators, motors, fans, solenoids, valves, pumps, transmitters, signal emitters, lights, and so forth.

The computing machine may operate in a networked environment using logical connections through the network interface to one or more other systems or computing machines across the network. The network may include wide area networks (WAN), local area networks (LAN), intranets, the Internet, wireless access networks, wired networks, mobile networks, telephone networks, optical networks, or combinations thereof. The network may be packet switched, circuit switched, of any topology, and may use any communication protocol. Communication links within the network may involve various digital or an analog communication media such as fiber optic cables, free-space optics, waveguides, electrical conductors, wireless links, antennas, radio-frequency communications, and so forth.

The processor may be connected to the other elements of the computing machine or the various peripherals discussed herein through the system bus. It should be appreciated that the system bus may be within the processor, outside the processor, or both. According to some embodiments, any of the processor, the other elements of the computing machine, or the various peripherals discussed herein may be integrated into a single device such as a system on chip (“SOC”), system on package (“SOP”), or ASIC device.

Embodiments may comprise a computer program that embodies the functions described and illustrated herein, wherein the computer program is implemented in a computer system that comprises instructions stored in a machine-readable medium and a processor that executes the instructions. However, it should be apparent that there could be many different ways of implementing embodiments in computer programming, and the embodiments should not be construed as limited to any one set of computer program instructions. Further, a skilled programmer would be able to write such a computer program to implement one or more of the disclosed embodiments described herein. Therefore, disclosure of a particular set of program code instructions is not considered necessary for an adequate understanding of how to make and use embodiments. Further, those skilled in the art will appreciate that one or more aspects of embodiments described herein may be performed by hardware, software, or a combination thereof, as may be embodied in one or more computing systems. Moreover, any reference to an act being performed by a computer should not be construed as being performed by a single computer as more than one computer may perform the act.

The example embodiments described herein can be used with computer hardware and software that perform the methods and processing functions described previously. The systems, methods, and procedures described herein can be embodied in a programmable computer, computer-executable software, or digital circuitry. The software can be stored on computer-readable media. For example, computer-readable media can include a floppy disk, RAM, ROM, hard disk, removable media, flash memory, memory stick, optical media, magneto-optical media, CD-ROM, etc. Digital circuitry can include integrated circuits, gate arrays, building block logic, field programmable gate arrays (FPGA), etc.

Reagents, tools, and/or instructions for performing the methods described herein can be provided in a kit. In certain embodiments, there is provided a kit for use in a method for selecting a treatment for a subject having a cancer as described herein and/or for use in a method for predicting the responsiveness of a subject with cancer to a therapeutic agent as described herein and/or for use in a method of determining a clinical prognosis for a subject with cancer as described herein.

The kit may include reagents for collecting a tissue sample from a patient, such as by biopsy, and reagents for processing the tissue. Thus, the kit may include suitable fixatives, such as formalin and embedding reagents, such as paraffin. The kit can also include one or more reagents for performing an expression level analysis, such as reagents for performing nucleic acid amplification, including RT-PCR and qPCR, NGS (RNA-seq), northern blot, proteomic analysis, or immunohistochemistry to determine expression levels of biomarkers in a sample of a patient. For example, primers for performing RT-PCR, probes for performing northern blot analyses or bDNA assays, and/or antibodies or aptamers, as discussed herein, for performing proteomic analysis such as Western blot, immunohistochemistry and ELISA analyses can be included in such kits. Appropriate buffers for the assays can also be included. Detection reagents required for any of these assays can also be included. The kits may be array or PCR based kits for example and may include additional reagents, such as a polymerase and/or dNTPs for example. The kits featured herein can also include an instruction sheet describing how to perform the assays for measuring expression levels.

The kit may include one or more primer pairs and/or probes complementary to at least one gene selected from Table A or Table B. In certain embodiments, according to all aspects of the invention, the kits may include one or more probes or primers (primer pairs) designed to hybridize with the target sequences or full sequences listed in Table A or Table B and thus permit expression levels to be determined. The probes and probesets identified in Table A and Table B may be employed according to all aspects of the invention.

The kits may include primers/primer pairs/probes/probesets to form any of the gene signatures specified herein.

The kits may also include one or more primer pairs complementary to a reference gene.

Such a kit can also include primer pairs complementary to at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 of the genes listed in Table A and/or primer pairs complementary to at least 2, 3, 4, 5, 6, 7, 8, 9, 10, 11, 12, 13, 14, or 15 of the genes listed in Table B.

There is provided a kit for use in a method for selecting a treatment for a subject having a cancer as described herein and/or for use in a method for predicting the responsiveness of a subject with cancer to a therapeutic agent as described herein and/or for use in a method of determining a clinical prognosis for a subject with cancer as described herein comprising one or more primers and/or primer pairs for amplifying and/or which specifically hybridize with at least one gene, full sequence or target sequence selected from Table A or Table B. There is also provided a kit for use in a method for selecting a treatment for a subject having a cancer as described herein and/or for use in a method for predicting the responsiveness of a subject with cancer to a therapeutic agent as described herein and/or for use in a method of determining a clinical prognosis for a subject with cancer as described herein comprising one or more probes that specifically hybridize with at least one gene, full sequence or target sequence selected from Table A or Table B.

The probes and probesets also constitute separate aspects of the invention. By “probeset” is meant the collection of probes designed to target (by hybridization) a single gene.

The invention also relates to a kit for use in the methods described herein comprising one or more antibodies or aptamers as described above and which are useful in the methods of the invention.

Informational material included in the kits can be descriptive, instructional, marketing or other material that relates to the methods described herein and/or the use of the reagents for the methods described herein. For example, the informational material of the kit can contain contact information, e.g., a physical address, email address, website, or telephone number, where a user of the kit can obtain substantive information about performing a gene expression analysis and interpreting the results.

The kit may further comprise a computer application or storage medium as described above.

The example systems, methods, and acts described in the embodiments presented previously are illustrative, and, in alternative embodiments, certain acts can be performed in a different order, in parallel with one another, omitted entirely, and/or combined between different example embodiments, and/or certain additional acts can be performed, without departing from the scope and spirit of various embodiments. Accordingly, such alternative embodiments are included in the scope of the invention as described herein.

Although specific embodiments have been described above in detail, the description is merely for purposes of illustration. It should be appreciated, therefore, that many aspects described above are not intended as required or essential elements unless explicitly stated otherwise.

Modifications of, and equivalent components or acts corresponding to, the disclosed aspects of the example embodiments, in addition to those described above, can be made by a person of ordinary skill in the art, having the benefit of the present disclosure, without departing from the spirit and scope of embodiments defined in the following claims, the scope of which is to be accorded the broadest interpretation so as to encompass such modifications and equivalent structures.

### DESCRIPTION OF THE FIGURES

FIG. 1: Identification of molecular subgroups of HGSOC

A. Heat map showing unsupervised hierarchical clustering of gene expression data using the 1040 most variable genes in the Edinburgh 265 high grade serous ovarian carcinomas. Gene expression across all samples is represented horizontally. Functional processes corresponding to each gene cluster are labelled along the right of the figure. Angio (blue), Immune (green), and Angio_Immune (red) subgroups are labelled for each of the sample clusters, and colour coded along the top as described in the legend box. A gene expression signature to detect each of the subgroups was generated. B. Kaplan-Meier Progression-Free Survival analysis of subgroups as defined by unsupervised clustering analysis of Edinburgh 265 HGSOC Samples. Additionally Kaplan-Meier overall survival analysis of subgroups as defined by unsupervised clustering analysis of Edinburgh 265 HGSOC Samples. C. Kaplan-Meier to show the prognostic utility of the Angio_Immune subgroup in HGSOC (PFS HR 1.4 (1.092 to 1.880) p=0.0256 and OS HR 1.4 (1.05-1.87) p=0.0224). D. Molecular subgroups are dynamic in the context of chemotherapy. The effect of chemotherapy treatment on 48 matched pre-chemotherapy and post-chemotherapy samples and analysis of subgroup switching based on assessment of the 3 gene signature scores (22 Angio signature; 63 Immune signature and 45 Angio_immune signature) generated from the treatment naive Discover dataset.

FIG. 2: Cisplatin resistant cell line models have elevated 45 gene signature score

A. Generation of Cisplatin resistant OVCAR3 cell lines. 10-day colony formation assay assessing sensitivity of OVCAR3-WT and OVCAR3-CP cells to increasing concentrations of cisplatin. B. Cisplatin sensitive and resistant A2780 cell line models were scored with each of the 3 gene signatures and scores plotted in a bar graph, Angio_Immune (p=0.0057), Angio (p=0.3959) and Immune (p=0.0124). C. Cisplatin sensitive and resistant OVCAR3 cell line models were scored with each of the 3 gene signatures and scores plotted, Angio_Immune (p=0.0244), Angio (p=0.2478) and Immune (p=0.028). D. Western blot analysis showing increased MAPK signalling in the A2780 and OVCAR3 cisplatin resistant cells compared to cisplatin sensitive counterparts. E. Colony formation assay with cisplatin in 15 ovarian cell lines, plotting 45-gene signature scores based on median centred IC50 doses (AUC 0.7917 (0.6350-0.9483), p=0.0008) and plotting IC50 doses based on median centred signature scores (AUC 0.6838 (0.5184-0.8491), p=0.0377).

FIG. 3: The Angio_Immune group is driven by the MAPK pathway

A. Semi-supervised clustering analysis was performed on the Discovery dataset using the 3 public gene lists. Genes separating the ovarian samples were selected for further analysis. These were combined and a compilation gene list compiled and semi-supervised analysis of the Discovery dataset performed again. B. Venn diagrams illustrating the overlap of the ‘MEK ON’ population with the 3 gene signatures. This demonstrated 77% overlap with the Angio_immune subgroup. C. TOGA ovarian samples were scored with the 3 ovarian gene signatures. Correlation with the gene signatures and the pMAPK RRPA data was investigated using ROC analysis. Each of the 3 gene signature scores of TOGA samples were median centered and defined as being High and Low scores. A ROC curve was generated using the binary signature scores and the continuous pMAPK expression (TOGA) in 237 samples. A statistically significant result was found with the 45-gene signature (p=0.04786) but not with the 63 or 22 gene signatures (p=0.4337 and p=0.4109 respectively). D. (i) Colony formation assay with Trametinib in 16 ovarian cell lines, plotting 45-gene signature scores based on median centred IC50 doses (AUC 0.7234 (0.5778-0.8690), p=0.0090) and plotting IC50 doses based on median centred signature scores (AUC 0.7147 (0.5674-0.8620), p=0.0117). ii. 881 cell lines from the Sanger center were scored with the 45 gene AngioImmune signature and correlated to IC50 response to Trametinib. AngioImmune gene signature scores were plotted based on median centred IC50 doses and IC50 doses were plotted based on median centred signature scores. iii. 760 cell lines from the Sanger center were scored with the 45 gene AngioImmune signature and correlated to IC50 response to Selumetinib. AngioImmune gene signature scores were plotted based on median centred IC50 doses and IC50 doses were plotted based on median centred signature scores.

FIG. 4: The MEK signature is altered by KRAS status and MEK inhibitor

A. The 45-gene, 22-gene and 63 gene signature scores from the E-GEOD-55624 data whereby SW480 cells (KRAS G12D) were treated with a MEK inhibitor for 4 and 16 hours. The Angio_Immune signature scores was significantly reduced post MEK inhibitor treatment at both 4 and 16 hours (p=0.0055 and p=0.0143 respectively). B. Differences in the 3 gene signatures between HCT116 (KRAS MT) and HKH2 cells (KRAS WT) using the E-MEXP-3557 dataset. The 45-gene signature scores were elevated in KRAS mutant cells. C. E-GEOD 12764: MCF10 breast cells transfected with empty vector (EV) or HRAS or MEK1 confirmed elevated 45-gene signature scores in the HRAS and MEK1 mutants (p=0.0004 and p<0.0001 respectively). D. Inhibition of MEK with Trametinib decreases the 45-gene Angio_Immune signature score in OVCAR3 cells (p=0.0011).

FIG. 5: MEK inhibition in cisplatin resistant OVCAR3 cells, re-sensitises to cisplatin

A. 10 day colony formation assay assessing sensitivity of OVCAR3-WT and OVCAR3-CP cells to increasing concentrations of Cisplatin and MEK inhibitor as single agents. B. 10 day colony formation assay assessing sensitivity of OVCAR3-WT and OVCAR3-CP cells to increasing concentrations of Trametinib (GSK1120212). Table shows IC50 values for OVCAR3-WT and OVCAR3-CP cells for Cisplatin and Cisplatin in combination with Trametinib.

FIG. 6: The Angioimmune subgroup is associated with increased EMT signalling

A. Box and whisker plots depicting the expression of EMT related genes across the 3 HGSOC molecular subgroups. Expression of VIM, AXL, TWIST1, SNAIL and SLUG is enhanced in the Angio_Immune subgroup (p<0.0001). B. Box and whisker plot of 45-gene signature scores in MCF7 control and SNAIL overexpressing cells (E-GEOD-58252). The 45-gene signature is enhanced by SNAIL overexpression (p=0.0004).

FIG. 7: Activation of the EMT phenotype is enhanced in Cisplatin resistant ovarian cell lines

A. 10-day colony formation assay assessing sensitivity of OVCAR3-WT and OVCAR3-CP cells to increasing concentrations of cisplatin (left panel). Western blot analysis showing increased MAPK signalling in the OVCAR4 cisplatin resistant cells compared to cisplatin sensitive counterparts (right panel). B. Western blot analysis showing activation of EMT signalling in OVCAR3 CP and OVCAR4 CP (cisplatin resistant) with increased protein expression of Vimentin, N-cadherin and SLUG whilst decreasing protein expression of E-cadherin. B-actin was used as a loading control. C. Quantitative real-time PCR (qRT-PCR) expression of EMT markers (N-cadherin, SLUG, SNAIL, Vimentin, TWIST and TGF-β3) in cisplatin resistant OVCAR3 cells. Fold change plotted relative to wildtype counterparts. D. Quantitative real-time PCR (qRT-PCR) expression of EMT markers (N-cadherin, SLUG, SNAIL, Vimentin, TWIST and TGF-β3) in cisplatin resistant OVCAR4 cells. Fold change plotted relative to wildtype counterparts. E. Bar charts to show the fold change increase in migration of OVCAR3 and OVCAR4 cisplatin resistant cells compared to the wildtype ovarian cell lines.

FIG. 8: The EMT signature predicts resistance to inhibitors of the SRC pathway

A. Representative western blot showing levels of phosphorylated ERK, and SRC following treatment of TOV112D cells with 1 μM SRC inhibitor, Saracatinib for 3, 6, 12 and 24 hours. Total ERK and total SRC expression are also shown. Beta actin was used as a loading control. Representative western blot showing levels of phosphorylated SRC and ERK following treatment of TOV112D cells with 1 μM MEK inhibitor, Trametinib for 3, 6, 12 and 24 hours. Total SRC and total ERK expression are also shown. Beta-actin was used as a loading control. B. Box and whisker plots showing differences in the 45-gene signature scores between SRC inhibitor resistant and sensitive cells.

FIG. 9: The MEK subgroup is present in colon cancer and the EMT signature is prognostic

A. Heatmap representation of semi-supervised analysis of the MARISA dataset (GSE40967) using the Angio_Immune genes. Five individual clusters were identified, with Sample Cluster 3 (highlighted by the red box) defining the MEK driven subgroup. B. Kaplan-Meier to show the relapse-free survival of the five sample cluster groups. The MEK driven group represents poor prognosis in comparison to the other subgroups (p=0.037). C. Kaplan-Meier to show the relapse-free survival using the 45-gene signature scores from Marisa. The MEK ON group represents poor prognosis in comparison to the MEK OFF group (AUC 1.5949 (1.0951-2.3228), p=0.0063). D. Kaplan-Meier to show the disease-free survival using the 45-gene signature scores in the Jorissen dataset (GSE14333). The MEK ON group represents poor prognosis in comparison to the MEK OFF group (AUC 2.4543 (1.2049-4.999), p=0.0014).

FIG. 10: The MEK subgroup is present in NSCLC cancer and the EMT signature is prognostic

A. Heatmap representation of semi-supervised analysis of the Okayama dataset (GSE31210) using the Angio_Immune genes. Five individual clusters were identified, with Sample Cluster 4 (highlighted by the red box) defining the MEK driven subgroup. B. Kaplan-Meier to show the relapse-free survival of the five sample cluster groups. The MEK driven group represents poor prognosis in comparison to the other subgroups (p=0.0004). C. Kaplan-Meier to show the progression-free survival using the 45-gene signature scores from Okayama. The SIG POS group represents poor prognosis in comparison to the SIG NEG group (AUC 3.045 (1.631-5.686), p=0.0005). D. Kaplan-Meier to show the overall survival using the 45-gene signature scores in the Okayama dataset. The SIG POS group represents poor prognosis in comparison to the SIG NEG group (AUC 2.872 (1.271-6.489), p=0.0312).

FIG. 11: The 15 gene signature predicts cisplatin response and is elevated in cisplatin resistant cells

A. Kaplan-Meier to show the prognostic utility of the 15-gene Angio_Immune subgroup in HGSOC (PFS HR=1.3564 [1.0156-1.8117]; p=0.0279 and OS HR=1.3464 [0.9901-1.8308]; p=0.0441). B. Colony formation assay with cisplatin in 15 ovarian cell lines, plotting 15-gene signature scores based on median centred IC50 doses (AUC 0.6905 (0.5254-8556), p=0.0290) and plotting IC50 doses based on median centred signature scores (AUC 0.6897 (0.5326-0.8468), p=0.02932). C. Cisplatin sensitive and resistant OVCAR3 cell line models were scored with the 15-gene signature and scores plotted in a box and whisker plot, (p=0.046).

FIG. 12: Association of the 15 gene signature with the MAPK pathway

A. Differences in the 15-gene signature between HCT116 (KRAS MT) and HKH2 cells (KRAS WT) using the E-MEXP-3557 dataset. The 15-gene signature scores were elevated in KRAS mutant cells (p=0.0443). B. E-GEOD 12764: MCF10 breast cells transfected with empty vector (EV) or HRAS or MEK1 confirmed elevated 15-gene signature scores in the HRAS and MEK1 mutants (p<0.0001). C. Inhibition of MEK with Trametinib decreases the 15-gene Angio_Immune signature score in OVCAR3 cells (p=0.0023). D. Colony formation assay with Trametinib in 15 ovarian cell lines, plotting 15-gene signature scores based on median centred IC50 doses (AUC 0.850 (0.7366-0.9636), p<0.0001) and plotting IC50 doses based on median centred signature scores (AUC 0.737 (0.5820-0.8974, p=0.006495).

FIG. 13: The 15 gene signature is elevated by EMT

Box and whisker plot of 15-gene signature scores in MCF7 control and SNAIL overexpressing cells (E-GEOD-58252). The 15-gene signature is enhanced by SNAIL overexpression (p=0.0015).

FIG. 14: The 15-gene EMT signature predicts resistance to inhibitors of the SRC pathway

A and B. Box and whisker plots showing differences in the 15-gene signature scores between SRC inhibitor resistant and sensitive cells following treatment with Saracatinib. Median centered on signature score (AUC 0.7289 (0.5544-0.9035), p=0.01454) or median centered on IC50 of Saracatinib (AUC 0.7698 (0.6054-0.9343), p=0.004076).

FIG. 15: The 15 gene signature is prognostic in colon cancer

A. Kaplan-Meier to show the disease-free survival using the 15-gene signature scores in the Jorissen dataset (GSE14333). The MEK ON group represents poor prognosis in comparison to the MEK OFF group (p=0.0328). B. Kaplan-Meier to show the relapse-free survival using the 15-gene signature scores from Marisa. The MEK ON group represents poor prognosis in comparison to the MEK OFF group (p=0.0161).

FIG. 16: The 15 gene signature is prognostic in NSCLC cancer

A. Kaplan-Meier to show the progression-free survival using the 15-gene signature scores from Okayama. The SIG POS group represents poor prognosis in comparison to the SIG NEG group (p=0.0024). B. Kaplan-Meier to show the overall survival using the 15-gene signature scores in the Okayama dataset. The SIG POS group represents poor prognosis in comparison to the SIG NEG group (p=0.0396).

FIG. 17: Scatterplots of combined variance-intensity rank of the 19920 Entrez gene IDs in Ovarian Cancer, Colon Cancer, Lung Cancer and Melanoma

FIG. 18: Intersection of top ranked genes within different disease indications

FIG. 19: C-index figures within cross validation in the training dataset

FIG. 20: Curve of sensitivity and specificity to determine threshold for classification of MEK signature (X: 0.5899, Y: 1.567)

FIG. 21: Functional analysis of gene ontology (GO) biological processes (BP) for probeset clusters identified in hierarchical clustering.

FIG. 22: Core set analysis: Tothill_HR_Final_Core Set Analysis_15 Gene

FIG. 23: Core set analysis: ICON7_HR_Final_Core Set Analysis_15 Gene

FIG. 24: Minimum gene set analysis: Tothill_Validation_Min Gene Analysis_15 Gene

FIG. 25: Minimum gene set analysis: ICON7_Validation_Min Gene Analysis_15 Gene

FIG. 26: Core set analysis: Tothill_HR_Final_Core Set Analysis_45 Gene

FIG. 27: Core set analysis: ICON7_HR_Final_Core Set Analysis_45 Gene

FIG. 28: Minimum gene set analysis: Tothill_Validation_Min Gene Analysis_45 Gene

FIG. 29: Minimum gene set analysis: ICON7_Validation_Min Gene Analysis_45 Gene

FIG. 30: ALM201 reverses mesenchymal markers in the Kuramochi and OVCAR3 cisplatin-resistant cells
A. Western Blot analysis showing reversal of the EMT pathway and downregulation of the MAPK pathway by ALM201 in ovarian Kuramochi cells.
B. Western blot demonstrating activity of ALM201 in reversing EMT markers, downregulation of the MAPK pathway following addition of 1 nM and 10 nM ALM201 at 24 hour treatment time point in the OVCAR3 cisplatin resistant cell line.
C. 10-day colony formation assay assessing sensitivity of OVCAR3-WT, OVCAR3-CP, OVCAR4-WT and OVCAR4-CP cells to increasing concentrations of ALM201. Table shows IC50 values for OVCAR3-WT and OVCAR3-CP cells for Cisplatin and Cisplatin in combination with ALM201.
D. xCELLigence migration and invasion assay illustrating that 0.1 nM, 1 nM and 10 nM ALM201 inhibits migration (p=0.08544, p=0.015522 and p=0.036739, respectively) and invasion (p=0.0211, p=0.0026 and p=0.3373, respectively) in the OVCAR3 platinum resistant cell line.

FIG. 31: The 45 gene and 15 gene signatures are predictive of response to MEK inhibitors

A. 739 cell lines from ‘The Genomics of Drug Sensitivity in Cancer Project’ (http://www.cancerrxgene.org/) were scored with the 45-gene AngioImmune signature and correlated to IC50 response to Trametinib. AngioImmune gene signature scores were plotted based on median centred IC50 doses and IC50 doses were plotted based on median centred signature scores. B. 759 cell lines from the ‘The Genomics of Drug Sensitivity in Cancer Project’ were scored with the 45-gene AngioImmune signature and correlated to IC50 response to Selumetinib. AngioImmune gene signature scores were plotted based on median centred IC50 doses and IC50 doses were plotted based on median centred signature scores. C. 739 cell lines from ‘The Genomics of Drug Sensitivity in Cancer Project’ (http://www.cancerrxgene.org/) were scored with the 15-gene AngioImmune signature and correlated to IC50 response to Trametinib. AngioImmune gene signature scores were plotted based on median centred IC50 doses and IC50 doses were plotted based on median centred signature scores. D. 760 cell lines from the ‘The Genomics of Drug Sensitivity in Cancer Project’ were scored with the 15-gene AngioImmune signature and correlated to IC50 response to Selumetinib. AngioImmune gene signature scores were plotted based on median centred IC50 doses and IC50 doses were plotted based on median centred signature scores.

FIG. 32: The 45 gene signature is predictive of response to taxanes

A. Scatter plot of 45-gene signature scores across two clinical groups, PSA responders and PSA non-responders. B. Kaplan-Meier to show patient survival using the 45-gene signature scores in response to taxane based chemotherapy in prostate cancer. The EMT positive group (blue) represents the good prognosis group who respond well to taxane in comparison to the EMT negative group (red) C. Table representing the breakdown of PSA responders and non-responders who are EMT positive and negative within the pilot cohort.

FIG. 33: The 15 gene signature is predictive of response to taxanes

A. Scatter plot of 15-gene signature scores across two clinical groups, PSA responders and PSA non-responders. B. Kaplan-Meier to show patient survival using the 15-gene signature scores in response to taxane based chemotherapy in prostate cancer. The EMT positive group (blue) represents the good prognosis group who respond well to taxane in comparison to the EMT negative group (red) C. Table representing the breakdown of PSA responders and non-responders who are EMT positive and negative within the pilot cohort.

FIG. 34: EMT signature is prognostic in Prostate Cancer, predicting disease recurrence and poor prognosis post radical surgery

Kaplan-Meier to show prognostic relevance of the 15-gene signature scores in predicting biochemical recurrence in prostate cancer. The EMT positive group (15-gene signature high) (blue) represents the poor prognosis group who have poorer survival and greater chance of biochemical recurrence in comparison to the EMT negative group (15-gene signature low) (green).

FIG. 35: EMT signature is prognostic in Prostate Cancer, predicting disease recurrence, metastasis and poor prognosis post radical radiotherapy

A. Kaplan-Meier to show prognostic relevance of the 15-gene signature scores in predicting biochemical recurrence in prostate cancer. The EMT positive group (15-gene signature high) (green) represents the poor prognosis group who have poorer survival and greater chance of biochemical recurrence in comparison to the EMT negative group (15-gene signature low) (blue). B. Kaplan-Meier to show prognostic relevance of the 15-gene signature scores in predicting biochemical recurrence in prostate cancer. The EMT positive group (15-gene signature high) (green) represents the poor prognosis group who have poorer survival and greater chance of metastatic progression in comparison to the EMT negative group (15-gene signature low) (blue).

FIG. 36: The 15 gene signature is prognostic in multiple diseases

A. Kaplan-Meier to show the disease-free survival using the 15-gene signature scores in the TOGA RNA-seq dataset across multiple diseases (shown in B) The MEK/EMT ON group represents poor prognosis in comparison to the MEK/EMT OFF group). B. Table showing hazard ratios and statistical significance of EMT biomarker across individual diseases.

FIG. 37: Platinum therapy demonstrates a selection pressure for an angiogenesis enriched tumor microenvironment A. CD31 Immunohistochemistry (IHC) quantification of micro vessel density (MVD) of 12 matched pairs of patient samples pre- and post-platinum-based chemotherapy. Specifically, samples were obtained at diagnosis and then from debulking surgery following relapse after completion of chemotherapy. This data shows that chemotherapy creates a selection pressure for an angiogenesis-dependent tumour microenvironment. Tumours that have relapsed following platinum therapy have acquired higher micro-vessel density (MVD) compared to their treatment naïve pair (p-value=<0.0001). B. The post-platinum treatment patient samples have higher 15 gene signature score than is paired platinum-naïve tissue (p-value: 0.0094)

FIG. 38: Angiogenesis assay using Matrigel plugs in Athymic nude mice illustrating that the OVCAR3 cisplatin resistant cell have hallmarks of vascular mimicry

OVCAR3 and OVCAR4 HGSOC cell lines were continuously exposed to increasing concentrations cisplatin over 6 months to generate cisplatin resistant OVCAR3CP and OVCAR4CP cells respectively. In-vivo matrigel plug assay to demonstrate the MVD in the OVCAR3 isogenic cell lines. H&E quantification of MVD of the OVCAR3 isogenic cell lines shows that co-culturing the OVCAR3 CP cell lines with ECFCs have a higher MVD (p-value: 0.0041) compared to the parental cell lines (p-value: 0.8712). The OVCAR3 CP cell lines has a higher 15-gene signature score relative to the OVCAR3 WT cell line (p-value: 0.046)

FIG. 39: Platinum resistant cell lines are associated with expression of ligands and their reciprocal RTK associated with the angiogenesis process.

Cytokine array shows that the platinum resistant OVCAR3 (A) and OVCAR4 (B) have higher expression of cytokines that a key regulators of angiogenesis

(C) Western blot showing that VEGFa protein expression levels are higher in OVCAR3 and OVCAR4 cisplatin-resistant cells in comparison to the OVCAR3 and OVCAR4 cisplatin-naïve cells.

FIG. 40: Table depicting the expression of Receptor tyrosine kinases (RTKs) and associated ligands and genes involved in the EMT in the AngioImmune molecular subgroup and the pre/post chemotherapy samples.

A. AngioImmune subgroup is characterised by expression of RTKs that are key regulators of the mesenchymal and proliferative phenotype in ovarian cancer compared to the other 2 subgroups.

B. Pre chemotherapy samples verses post-chemotherapy samples. RTKs shown represent those which were statistically associated either by ROC analysis (AUC) or student t-test where indicated.

FIG. 41: Platinum resistant cell lines are associated with expression of RTK associated with the angiogenesis process

A. pRTK array shows that the OVCAR3 cisplatin-resistant cell line has higher basal expression of pRTK relative to the platinum-naïve OVCAR3 pair.

B. Further validation of the pRTK array by western blot shows basal upregulation of phospho-VEGFR2, VEGFR3, PDGFRα and phospho-AXL in the OVCAR3 cisplatin-resistant relative to the OVCAR3 cisplatin-naïve pair

FIG. 42: TKIs have specificity for platinum resistant OVCAR3 isogenic cell line

A. 10-day colony formation assay of Cediranib in the OVCAR3 isogenic cell lines demonstrates sensitivity for the OVCAR3 cisplatin-resistant (IC50 1.194) relative to the OVCAR3 cisplatin-naïve cell line (IC50 4.994).

B. 10-day colony formation assay of Nintedanib in the OVCAR3 isogenic cell lines demonstrates sensitivity for the OVCAR3 cisplatin-resistant (IC50 3.777) relative to the OVCAR3 cisplatin-naïve cell line (IC50>10).

FIG. 43: TKIs that target downstream RTKs lead to inhibition of tumor VEGFa expression.

A CellTiter Glo assay was carried out to determine the IC50 for Cediranib (IC50 5.569 μM at 48 hour time point) and Nintedanib (IC50 9.097 μM at 48 hour time point).

B. Western blot showing that VEGFa protein expression levels are down-regulated in OVCAR3 and OVCAR4 cisplatin-resistant cells treated with an IC50 concentration of Cediranib and Nintedanib.

FIG. 44: Angiogenesis assay using Matrigel plugs in Athymic nude mice: Effect of Bevacizumab on MVD

In-vivo matrigel plug assay to demonstrate the effect of bevacizumab on MVD in the OVCAR3 isogenic cell lines. IF quantification of MVD of the OVCAR3 isogenic cell lines shows that co-culturing the OVCAR3 CP cell lines with ECFCs have a higher MVD (p-value: 0.0024) compared to the parental cell lines (p-value: 0.84525).

### EXAMPLES

The present invention will be further understood by reference to the following experimental examples.

### Example 1

MEK Activation is Associated with a Molecular Subgroup in High Grade Serous Ovarian Cancer

Epithelial ovarian cancer (EOC) ranks among the top ten diagnosed and top five deadliest cancers in most countries (Ferlay et al., 2010). Continental rates are highest in Europe (10.1 per 100,000) with 41,448 deaths from ovarian cancer in 2008, representing 5.5% of all female cancer deaths in Europe. The high death rate is because most patients (>60%) are diagnosed at an advanced stage of disease (Stage III and IV) (Vaugh et al., 2012). The most common type of EOC is high-grade serous ovarian cancer (HGSOC) which accounts for at least 70% of cases, the majority of which are stage III and IV disease (Bowtell, 2010). Currently, the standard treatment used in initial management is cytoreductive surgery and adjuvant chemotherapy with a platinum-based regimen. However, despite an initial complete clinical-response rate of 65%-80%, most stage III and IV ovarian carcinomas relapse with an overall 5-year survival rate of only 10%-30% and a median survival of 2 to 3 years (www.cancerresearchuk.org). Classic clinicopathological factors, such as age, stage, residual tumour after surgery, differentiation grade and histopathological features, are currently the most important prognostic markers, but it is not possible to select optimal chemotherapy on an individual patient basis using these factors. Over the past 20 years there has been very little progress in the treatment of HGSOC, with five-year survival figures remaining unchanged for stage III and IV disease (www.cancerresearchuk.org).

A number of studies have tried to characterise the mechanisms of acquired resistance in ovarian cancer. Analysis of 135 spatially and temporally separated samples from 14 patients with HGSOC who received platinum-based chemotherapy found that NF1 deletion showed a progressive increase in tumour allele fraction during chemotherapy (Schwarz et al., 2015). This suggested that subclonal tumour populations are present in pre-treatment biopsies in HGSOC and can undergo expansion during chemotherapy, causing clinical relapse (Schwarz et al., 2015). Additionally alteration of the NF1 gene has been associated with innate cisplatin resistance in HGSOC, whereby 20% of primary tumours showed inactivation of the NF1 gene by mutation or gene breakage (Patch et al., 2015). Furthermore mutation of the RAS-MAPK has been associated with chemotherapy resistance in relapsed neuroblastomas (Eleveld et al., 2015). Additionally, in cell line models, the MAPK pathway has been implicated in cisplatin resistance in ovarian cancer (Benedetti et al., 2008) and in squamous cell carcinoma (Kong et al., 2015).

### Key Messages

MAPK is a pathway of innate and acquired resistance in High Grade Serous Ovarian Cancer (HGSOC).
After cisplatin treatment, samples switch molecular groups, move into a MAPK/EMT molecular group and become more angiogenic.
We have developed a 45 and 15 gene expression signature which can detect the MAPK molecular subgroup.
45 and 15 gene signatures predict sensitivity to drugs targeting the MAPK pathway: MEK inhibitors.
45 and 15 gene signatures predict resistance to SRC inhibitors.
45 and 15 gene signatures detect a cisplatin resistant group and predicts resistance to cisplatin.
45 and 15 gene signatures predict a bad prognosis molecular subgroup in colon cancer (CRC) and non-small cell lung cancer (NSCLC).

### Materials & Methods

### High Grade Serous Ovarian Cancer (HGSOC) Tumour Material

This study performed gene expression analysis of a cohort of 265 macrodissected ovarian cancer FFPE tissue samples sourced from the Edinburgh Ovarian Cancer Database. Ethical approval for Edinburgh dataset analysis was obtained from Lothian Local Research Ethics Committee 2 (Ref: 07/S1102/33).

This cohort of samples can be further described using the following inclusion criteria:
Primary ovarian, peritoneal or fallopian tube cancer
High grade serous histology
Treatment-naïve FFPE tissue samples
Matched pre chemotherapy and post-chemotherapy samples

### Prostate Tumour Material

Three separate prostate cancer cohorts were sourced and used to assess the association of EMT with Prostate Cancer prognosis.
1) Pilot Cohort—56 prostate biopsy samples with de novo metastatic disease, collected in collaboration with NI Biobank
2) Resection Cohort—multicentre retrospective cohort of 322 prostatectomy specimens collected from Wales Cancer Bank, University College Dublin, University of Surrey and Oslo University Hospital.
3) Biopsy Cohort—retrospective radiation cohort of 248 prostate biopsy specimens collected in collaboration with FASTMAN—Movember Centre of Excellence.

### Gene Expression Profiling of HGSOC and Prostate Samples

Total RNA was extracted from the macrodissected FFPE tumour samples using the Roche High Pure RNA Paraffin Kit (Roche Diagnostics GmbH, Mannheim, Germany) as described previously (Kennedy et al, 2011). Total RNA was amplified using the NuGEN WT-Ovation™ FFPE System (NuGEN Technologies Inc., San Carlos, Calif., USA). It was then hybridised to the Almac Ovarian Cancer DSA™ as described previously (Kennedy et al, 2011) or Prostate DSA™ for prostate samples (Tanney et al, 2008). Arrays were scanned using the Affymetrix Genechip® Scanner 7G (Affymetrix Inc., Santa Clara, Calif.).

### Data Preparation & Hierarchical Clustering

Quality Control (QC) of profiled samples was carried out using MAS5 pre-processing algorithm to assess technical aspects of the samples i.e. average noise and background homogeneity, percentage of present call (array quality), signal quality, RNA quality and hybridization quality. Distributions and Median Absolute Deviation of corresponding parameters were analyzed and used to identify possible outliers. Sample pre-processing was carried out using RMA (Irizarry et al, 2003). The pre-processed data matrix was sorted by decreasing variance, decreasing intensity and increasing correlation to cDNA yield. Following filtering of probe sets (PS) correlated with cDNA yield (to remove any technical bias in the expression data), hierarchical clustering analysis was performed (Pearson correlation distance and Ward's linkage methods (Ward et al, 1963). Subsets of the data matrix were tested for cluster stability using the GAP statistic (Tibshirani et al, 2001), which gives an indication of the within-cluster tightness and between-cluster separateness. The GAP statistic was applied to calculate the optimal number of sample clusters in each sub-matrix, while the stability of cluster composition was assessed using a partition comparison tool (Carriço et al, 2006; Pinto et al, 2008). The smallest number of PS generating the optimal sample cluster number was selected as the list of most variable PS to take forward for hierarchical cluster analysis.

### Functional Analysis of 3 Molecular Gene Clusters

To establish the functional significance of the gene clusters an enrichment analysis, based on the hypergeometric function (False Discovery Rate applied (Benjamini and Hochberg, 1995, J. R. Stat. Soc. 57:289:300)), was performed. Over-representation of biological processes and pathways were analysed for each gene group generated by the hierarchical clustering using Gene Ontology biological processes. Hypergeometric p-values were assessed for each enriched functional entity class. Functional entity classes with the highest p-values were selected as representative of the group and a general functional category representing these functional entities was assigned to the gene clusters based on significance of representation (i.e. p-value).

### Gene Selection for Signature Generation

Genes that are variable and highly expressed across multiple disease indications were determined prior to model development. The disease indications that were included in this evaluation were: ovarian cancer; colon cancer; lung cancer and melanoma. Two data sets per disease indication were assessed with the exception of prostate cancer where only one dataset was evaluated. Data sets were pre-processed using RMA and summarised to Entrez Gene ID level using the median of probe sets for each Entrez Gene ID on the Ovarian Cancer DSA™ Within each data set, Entrez Gene IDs were ranked based on the average rank by variance and mean intensity across samples (high rank=high variance, high mean intensity). A single combined rank value per gene was calculated based on the average variance-intensity rank within each disease indication. Genes with no expression level were removed from further analysis. Scatterplots were generated to show the combined variance-intensity rank of the 19920 Entrez gene IDs in the disease indications evaluated with two datasets (FIG. 17: (a) ovarian cancer; (b) colon cancer; (c) lung cancer; and (d) Melanoma) where the x and y axis represent the rank for the two data sets evaluated within each indication. A final classification of expressed genes as high/low rank was defined within each disease indication. Finally the overlap in high ranking genes across disease indications was determined and the top 75% ranked genes were identified. This list was then used as the starting list for signature generation (FIG. 18 & Table 1).

### Signature Generation of the 45-Gene and 15-Gene Signatures

The genes that had common high expression and variance in ovarian, colon, lung, melanoma and prostate were used as a starting set for model development. The Edinburgh ovarian cancer sample cohort was used to train the signature under 5 fold cross validation (CV) with 10 repeats. Partial least squares (PLS) (de Jong, 1993) was paired with Forward Feature Selection (FFS) to generate signatures for the top 75% ranked list. Table 4 indicates the weightings and bias for each probeset incorporated within the 45-gene signature (A) and the 15-gene signature (B)

### Model Selection and Signature Validation for the 15-Gene Signature

The C-index performance was calculated using the progression free survival (PFS) time endpoint and signature scores generated within cross validation for each evaluated signature length. This data was then used to determine the signature length at which optimal performance is reached with respect to association between signature scores and PFS. The highest C-index values were compared for signatures of length less than 100 and greater than 10 features. The signature with the shortest length and highest C-index within this subset was selected as the final model for identifying the subgroup.

FIG. 19 shows the C-index performance calculated under cross validation for the training set across all feature lengths, from a maximum of 5000 genes, removing 10% at each feature selection iteration until a minimum of 5 features. The C-index performance metric was the primary metric analysed for model selection. The C-index was significant across the majority of feature lengths in the training set (FIG. 19) and the C-index performance was highest at a feature length of 15 (56.62 [57.86-55.55]). Table 2 lists the Entrez Gene ID and corresponding Gene Symbol for the 15 gene signature.

A threshold was generated for classification of signature scores by using the value where the sum of sensitivity and specificity with respect to predicting the subtype in the training data is highest. This threshold was set at 0.5899 using the curve of sensitivity and specificity (FIG. 20). Samples with scores above the selected threshold would be classified as MEK ON whereas samples with scores below or equal to the threshold would be labelled as MEK OFF.

### Functional Analysis

Functional enrichment analysis of the selected model was performed using the Gene Ontology biological processes classification to gain an understanding of the underlying biology behind the selected signature. Table 3 presents the top 20 GO biological processes and GO terms from functional enrichment analysis of the signature, where the top 20 biological processes include:
Angiogenesis (p=2.09e-05)
Blood vessel development (p=5.55e-06)
Cell-cell junction organization (p=2.55e-05)

### Cell Culture and Reagents:

Human epithelial ovarian cancer cell lines OVCAR3 and OVCAR4 were obtained from the American Type Culture Collection. Tumour cells were cultured in RPMI (Gibco™ Life technologies) supplemented with 20% foetal calf serum (FCS) nd maintained in 5% CO2 at 37 C. Pharmaceutical grade cisplatin and bevacizumab were kindly provided by the Belfast City Hospital pharmacy department. Cediranib and Nintedanib were purchased through Selleckchem and re-suspended in DMSO to a stock concentration of 10 mM.

### Colony Formation Assays

Cells were seeded at predetermined densities, 24 hours later treated with drug, which was replenished every 3-4 days. After 10 days, cells were washed with PBS, fixed in methanol, stained with crystal violet and colonies counted. The surviving fraction (SF) for a given dose was calculated and dose-response curves plotted and IC50 generated using Graph Pad Prism™ 5. Receiver operator curves (ROC) were plotted by dicotomising the IC50 values based on the median of the IC50 and defining the higher IC50 values as resistant and the lower IC50 values as sensitive. The gene signatures associated with the cell lines were plotted based on sensitive and resistant cells. Additionally ROCs were plotted by dicotomising the signature scores based on the median of the scores and defining the higher signature score as signature positive and the lower signature scores as signature negative. The IC50s associated with the cell lines were plotted based on signature positive and signature negative cells.

### Migration Assay

The migration assay was performed using the xCELLigence RTCA DP system and carried out with CIM-plate 16 (ACEA Bioscience). Endothelial progenitor cell conditioned media, fresh endothelial media with growth factors (VEGF, IGF-1, bFGF, EGF) with 10% foetal bovine serum (FBS) and endothelial media with 10% foetal bovine serum (FBS) only, were the three chemo-attractant conditions used in the bottom chamber. 160 μl of the chemo-attractant was added to each bottom chamber of a CIM-plate 16. The CIM-Plate 16 is assembled by placing the top chamber onto the bottom chamber and snapping the two together. 30 μl pf serum-free medium is placed in the top chamber to hydrate and pre-incubate the membrane for 2 hours in the CO2 incubator at 37° C. before obtaining a background measurement. The protocol is optimised for the two paired cancer cell lines: OVCAR3, OVCAR4 parental and OVCAR3, OVCAR4 platinum resistant cell lines. Platinum resistant cell lines were washed ×3 with PBS, to remove cisplatin, and fresh platinum free media was added to the cells for 24 hours prior to carrying out the experiment. Cells were then grown in serum free medium for 2 hours prior to seeding. Cells are lightly trypsinised, pelleted and re-suspended at 100 μl, containing 50,000 cells, in serum-free medium. Once the CIM-Plate 16 has been equilibrated, it is placed in the RTCA DP station and the background cell-index values are measured. The CIM-Plate 16 is then removed from the RTCA DP station and the cells are added to the top chamber. The CIM-Plate 16 is placed in the RTCA DP station and migration is monitored every 5 minutes for several hours. Each experimental condition was performed in triplicate. For quantification of the migration rate, the slope of the curve was used to determine the rate if change in cell index. The average and standard deviation slope values were then quantified relative to the controls.

### Invasion Assay

The invasion assay was performed using the xCELLigence RTCA DP system and carried out with CIM-plate 16 (ACEA bioscience).

Normal cell media growth conditions (RPMI 1640, 1% L-Glut and 20% FCS) was the chemoattractant condition used in the bottom chamber. 160 μl of the chemoattractant was added to each bottom chamber of a CIM-plate 16. The CIM-Plate 16 is assembled by placing the top chamber onto the bottom chamber and snapping the two together. 20 μl Matrigel growth factor reduced (GFR) (phenol-red free) basement membrane matrix (Cornig, ref: 356231) was diluted in 400 μl optimem (serum free) giving a final working concentration of GFR Matrigel of 5%. 20 μl of the Matrigel-optimem master mix is placed in the top chamber to hydrate and pre-incubate the membrane for 2 hours in the CO2 incubator at 37° C. before obtaining a background measurement.

The protocol is optimized for the two paired cancer cell lines: OVCAR3, OVCAR4 parental and OVCAR3, OVCAR4 platinum resistant cell lines. Platinum resistant cell lines were grown for 24 hours in media containing 0.1 nM, 1 nM and 10 nM ALM201. On the experimental day, cells are washed ×1 with PBS. Cells are lightly trypsinized, pelleted and re-suspended at 100 μl, containing 50,000 cells, in optimem (serum-free) medium in the presence of 0.1 nM, 1 nM and 10 nM ALM201. Once the CIM-Plate 16 has been equilibrated, it is placed in the RTCA DP station and the background cell-index values are measured. The CIM-Plate 16 is then removed from the RTCA DP station and the cells are added to the top chamber. The CIM-Plate 16 is placed in the RTCA DP station and migration is monitored every 5 minutes for several hours.

Each experimental condition was performed in triplicate. For quantification of the migration rate, the slope of the curve was used to determine the rate if change in cell index. The average and standard deviation slope values were then quantified relative to that at the control condition.

### Proliferation (3-Day) Assay

The proliferation assay was performed using 6-well plates. The experiment was set-up with two controls mechanisms to ensure accuracy of results. Each cell line was seeded in duplicates and the experiment was performed in triplicate. For quantification of proliferation, cell numbers were counted manually using a coulter counter on day 1, day 2 and day 3 in three different concentrations of ALM201 (0.1 nM, 1 nM and 10 nM). Media is changed on day 2 and day 3 with fresh media containing the 3 concentrations of ALM201.

On day 0, each cell line was lightly trypsinized, counted and seeded at a concentration of 5×10⁴ per well in the presence of ALM201 (0.1 nM, 1 nM and 10 nM concentration). 2 mls of cells was added to each well in three 6 well plates (representing day 1, 2 and 3) and left to incubate for 24 hours in the CO2 incubator at 37° C. prior to counting cells for day 1, 48 hour incubation prior to count day 2 and 72 hour incubation prior to counting day 3.

At each time point, media was aspirated from the wells and wells were washed with PBS×1. 500 μl 5% trypsin was added to each well and incubated 3-5 mins. 1.5 mls media was added to each well to neutralise the trypsin. Cells were counted using the coulter counter. To estimate significance, the unpaired, two-tailed student T-test was calculated using the T-test calculator available on GraphPad Prism 5.0 software.

### Therapeutic Agents

Dasatinib (BMS354825), Saracatinib (AZD0530) and Trametinib (GSK1120212) were purchased from Selleck Chemicals, dissolved in DMSO to constitute a 10 mM stock solution, and stored at −20° C. Cisplatin was acquired from Belfast City Hospital Pharmacy department and diluted in PBS to produce a 10 μM stock solution. Cisplatin was stored at room temperature and protected from light.

### Generation of OVCAR3 and OVCAR4 Cisplatin Resistant Cell Lines

OVCAR3 and OVCAR4 cells were trypsinised and relevant cell numbers were seeded into P90 plates. Cells were allowed to adhere overnight. The following day media was removed and replaced with media containing 25 nM cisplatin. The concentration of cisplatin was increased every 2 weeks, doubling the concentration at every increment. Batches of cells were frozen every two weeks upon increasing the concentration of cisplatin. Once cells were stably growing at 200 nM cisplatin, sensitivity to cisplatin was tested by clonogenic assay. Cells were continuously grown in 200 nM cisplatin.

### Cell Viability Assay:

Cells were trysanised and counted using the Countess™ Automated Cell Counter. 5,000 cells were seeded into each well of a 96 well plate. Cells were allowed to adhere overnight and were then treated with titrated concentrations of cisplatin, cedirianib and nintedanib (10 μM to 0.005 μM concentration). Under sterile conditions in tissue culture, at the 24 hour time point, the drug-conditioned media was removed and replaced with 100 μl of fresh media. The 96 well plate was allowed to stand at room temperature for 20-30 mins. In the meantime the CellTiter-Glo Luminescent was allowed to thaw from the −20 freezer. Following the 20-30 minute incubation, 75 μl of the CellTiter-Glo Luminescent was added per well, this was then shaken for 2 minutes and then allowed to stand for 10 minutes. The analysis was performed using the Bioscience BioTek plate reader.

### In-Vivo Matrigel Plug Angiogenesis Assay:

All animal experiments were performed in conformity to UK Home Office Regulations (PPL2729) and with authorization from Queen's University Belfast Animal Welfare and Ethical Review Body (AWERB). Eight week-old male Athymic nude mice (Harlan Laboratories) were used. ECFC were inoculated at a high density of 2.45×106 and co-cultured with GFP-labelled OVCAR3 WT or GFP-labelled OVCAR3-CP at a density of 1.4×106. The GFP-labelled OVCAR3 WT and GFP-labelled OVCAR3-CP were seeded alone at a density of 1.4×106. Each condition was diluted in 10 μL of phenol red-free DMEM and re-suspended in 90 μL of growth factor-reduced Matrigel (Corning) and injected subcutaneously. After 8 days, mice were sacrificed under isoflurane anaesthesia using 31 gauge needless intraperitoneal (IP) administration of sodium pentobarbital at 200 mg/kg, and implants were removed and fixed in 4% formaldehyde overnight. Fixed Matrigel implants were then embedded in paraffin and 10 μm sections were prepared for staining. For 5 mg/Kg bevacizumab was administered IP once weekly for 14 days. Treatment was commenced on day 3. Mice were sacrificed after a 14-day treatment period as previously described.

### Human Angiogenesis Antibody Array

Protein lysate was obtained from OVCAR3 isogenic cell lines and analysed using the proteome profiler human angiogenesis array (R&D Systems, Abingdon, UK) in accordance with the manufacturer guidelines. Briefly, samples were adjusted to a final volume of 1.5 ml with array buffer and mixed with a detection antibody cocktail for 1 hour. After a membrane blocking step, samples containing antibody cocktail were added to membranes and left to incubate on a rocking platform overnight at 4° C. After several washes, membranes were incubated with strepavidin-horseradish peroxidase secondary antibody and spots were detected using a UVP bioimaging system (Millipore). Densitometry was quantified using Image J software.

### Human Cytokine Antibody Array:

Conditioned media was collected from OVCAR3 and OVCAR4 isogenic cell lines and added to the cytokine array (Abcam) membranes. The experiment was carried out in accordance with the manufacturer guidelines. Comparison between samples was performed using Image J densitometry software for a semi-quantitative comparison.

### Xenograft Immunostaining—Histologic and Immunofluorescence Analysis

IHC and immunofluorescence studies were conducted as previously describe [22]. Conventional H&E staining was done and examined by light microscopy. Immunofluorescence was done using anti-mouse CD31 (1:00; Baca), anti-rabbit α-smooth muscle actin (α-SMA; 1:100; Baca). For micro vessel counts, paraffin embedded tissues were sectioned and stained with anti-CD31 and anti-αSMA antibody. CD31 and αSMA stained vessels were then counted per high power field (200×) in three separate fields of three independent tumors from each group. Blood vessels associated with α-SMA-positive cells were considered mature. Sections were stained with α-SMA and with anti-CD31, which stains both mature and immature vessels.

Quantitative Real-Time Polymerase Chain Reaction (qRT-PCR)

Reverse transcription was performed using the First Strand cDNA synthesis kit (Roche). 500 ng of RNA was reverse transcribed according to manufacturer's instructions. Exon-spanning qPCR primers were designed using Roche Universal Probe Library Assay Design Centre and were used at a concentration of 0.5 μM. The following primer sequences were used:

N-cadherin
Forward
(SEQ ID NO. 278)
CTC-CAT-GTG-CCG-GAT-AGC
Reverse
(SEQ ID NO. 279)
CGA-TTT-CAC-CAG-AAG-CCT-CTA-C
SLUG
Forward
(SEQ ID NO. 280)
TGT-TGC-AGT-GAG-GGC-AAG-AA
Reverse
(SEQ ID NO. 281)
GAC-CCT-GGT-TGC-TTC-AAG-GA
SNAIL
Forward
(SEQ ID NO. 282)
ACC-ACT-ATG-CCG-CGC-TCT-T
Reverse
(SEQ ID NO. 283)
GGT-CGT-AGG-GCT-GCT-GGA-A
Vimentin
Forward
(SEQ ID NO. 284)
TGG-TCT-AAC-GGT-TTC-CCC-TA
Reverse
(SEQ ID NO. 285)
GAC-CTC-GGA-GCG-AGA-GTG
TWIST
Forward
(SEQ ID NO. 286)
AGC-TAC-GCC-TTC-TCG-GTC-T
Reverse
(SEQ ID NO. 287)
CCT-TCT-CTG-GAA-ACA-ATG-ACA-TC
TGF-β3
Forward
(SEQ ID NO. 288)
AAG-TGG-GTC-CAT-GAA-CCT-AA
Reverse
(SEQ ID NO. 289)
AAA-TTC-ACT-CTG-CCC-AGG-ACG
PUM1 (Housekeeping gene)
Forward
(SEQ ID NO. 290)
5′ CCA GAA AGC TCT TGA GTT TAT TCC 3′
Reverse
(SEQ ID NO. 291)
5′ CAT CTA GTT CCC GAA CCA TCT C 3′

To preform absolute quantification from qPCR, we used a standard curve method. The efficiency of each primer set was derived from the standard curve using the following equation:

E=10^{∧}(−1/slope)

The product of Reverse Transcription was diluted 1:10 in Nuclease Free Water (NFW). Each 10 μl PCR reaction, consisted of 0.5 μl of 10 μM Forward primer, 0.5 μl of 10 μM Reverse primer, 5 μl of 2× LightCycler® 480 SYBR Green I Master mix (Roche), 1.5 μl NFW and 2.5 μl diluted Reverse Transcription product. These 10 μl reactions were pipetted into wells of a LightCycler® 480 multiwell 96 plate (Roche), the plate was then sealed using clear adhesive film (Roche). The plate was placed into the LightCycler® 480 (Roche) and run with the following protocol. (95° C. for 10 mins, 45 cycles of; 95° C. for 15 secs, 55° C. for 30 secs and 72° C. for 30 secs, finishing with a melt curve for confirmation of primer specificity. All qPCR data was analysed using the LightCycler® 480 software provided by Roche. For analysis, the Cp value from a technical duplicate was calculated and the relative amount of a gene was calculated Cp value to an in-run standard curve. Each mean value was then normalised to the mean concentration of the housekeeping gene PUM1 within the corresponding sample, by dividing the concentration of the target gene by the concentration of the house keeping gene. Relative expression refers to the gene expression levels which have been normalised to the housekeeping gene and made relative to the associated control samples. From these normalized values, the fold changes for each gene were calculated and the average of three individual fold changes were derived from three independent experimental triplicates. The unpaired, two-tailed students T-test available on GraphPad Prism 5.0 software was used to detect statistical significance.

### Western Blotting

30 μg of protein lysates were mixed with LDS loading dye (Invitrogen) and Reducing Agent (Invitrogen) and denatured for 10 minutes at 70° C. Samples were briefly centrifuged, and loaded onto a Bolt 4-12% Bis-Tris gel and electrophoresed at 165 V for 1 hour 30 minutes using MOPS running buffer. SeeBlue Pre Stained protein ladder (Invitrogen) was used as a reference for protein size. After electrophoresis proteins were transferred onto immobilon-P PVDF membrane (Millipore) at 30 V for 2 hours using the XCell surelock mini-cell transfer system (Invitrogen). To ensure proper transferring of proteins onto membrane, the membrane was stained with Ponceau S solution (Sigma). Membranes were incubated in blocking solution (5% bovine serum albumin) for 1 hour at room temperature on a rocking platform in order to prevent non-specific binding of antibody to membrane. Membranes were then incubated in primary antibody overnight (see appendix 3 for dilutions) at 4° C. The following day, the membranes were washed 3 times in TBS-T for 10 minutes and incubated in secondary antibody at a 1:5000 dilution 1 hour 30 minutes at room temperature. Membranes were then washed 6 times for 5 minutes in TBS-T, and incubated for 5 minutes in Luminata Cresendo or Forte (Millipore) detection reagent. Analysis was performed using Alpha Innotech Imager FlourChem Software.

### Antibodies

ERK (Cell Signalling 2496)—monoclonal mouse antibody for total p44/42 MAP Kinase (ERK1/2), used at a 1:1000 dilution in 5% milk.

pERK (Cell Signalling 4370)—polyclonal rabbit antibody for p44 and p42 MAP Kinase (ERK1/2), used at a 1:1000 dilution in 5% BSA.

MEK (Cell Signalling 4694)—mouse monoclonal antibody for total MEK1/2, used at a dilution of 1:1000 in 5% milk.

pMEK (Cell Signalling 9121)—rabbit polyclonal antibody for phosph0-MEK1/2 at Ser217/221, used at a dilution of 1:1000 in 5% BSA.

SRC (Cell Signalling 2123)—rabbit polyclonal antibody for SRC, used at a dilution of 1:000 in 5% BSA.

pSRC (Cell Signalling 2101)—polyclonal rabbit antibody for phosphor-SRC at tyrosine 416, used at a dilution of 1:1000 in 5% BSA.

N-cadherin (Cell Signalling)—rabbit monoclonal, used at a dilution of 1:1000 in 5% milk.

E-cadherin (Cell Signalling 24E10)—rabbit monoclonal antibody used at a dilution of 1:1000 in 5% milk.

Vimentin (Cell Signalling R28)—rabbit monoclonal antibody to detect Vimentin, used at a dilution of 1:1000 in 5% milk.

SLUG (Cell Signalling C10G7)—rabbit monoclonal antibody to detect SLUG EMT marker, used at a dilution of 1:1000 in 5% milk.

VEGFa (Abcam)—rabbit polyclonal antibody to detect VEGFa, used at a dilution of 1:1000 in 5% BSA.

VEGFR1 (Abcam)—rabbit polyclonal antibody, a dilution of 1:500 in 5% BSA.

Phospho-VEGFR2 (Cell Signalling)—rabbit polyclonal antibody, a dilution of 1:500 in 5% BSA.

VEGFR2 (Cell Signalling)—rabbit polyclonal antibody, a dilution of 1:500 in 5% BSA.

PDGFRα (Cell Signalling)—rabbit polyclonal antibody, a dilution of 1:500 in 5% BSA.

Phospho-PDGFRβ (Cell Signalling)—rabbit polyclonal antibody, a dilution of 1:500 in 5% BSA.

Phospho-AXL (Cell Signalling)—rabbit polyclonal antibody, a dilution of 1:500 in 5% BSA.

AXL (Cell Signalling)—rabbit polyclonal antibody, a dilution of 1:500 in 5% BSA.

B-actin (Sigma A2228)—mouse monoclonal antibody detecting the N-terminus of β-actin, used at a dilution of 1:5000 in 5% milk.

Secondary antibodies—anti-rabbit and anti-mouse (Cell Signalling) were used at a dilution of 1:5000 in 5% milk.

### Results

### Identification of Molecular Subgroups of High Grade Serous Ovarian Cancer (HGSOC)

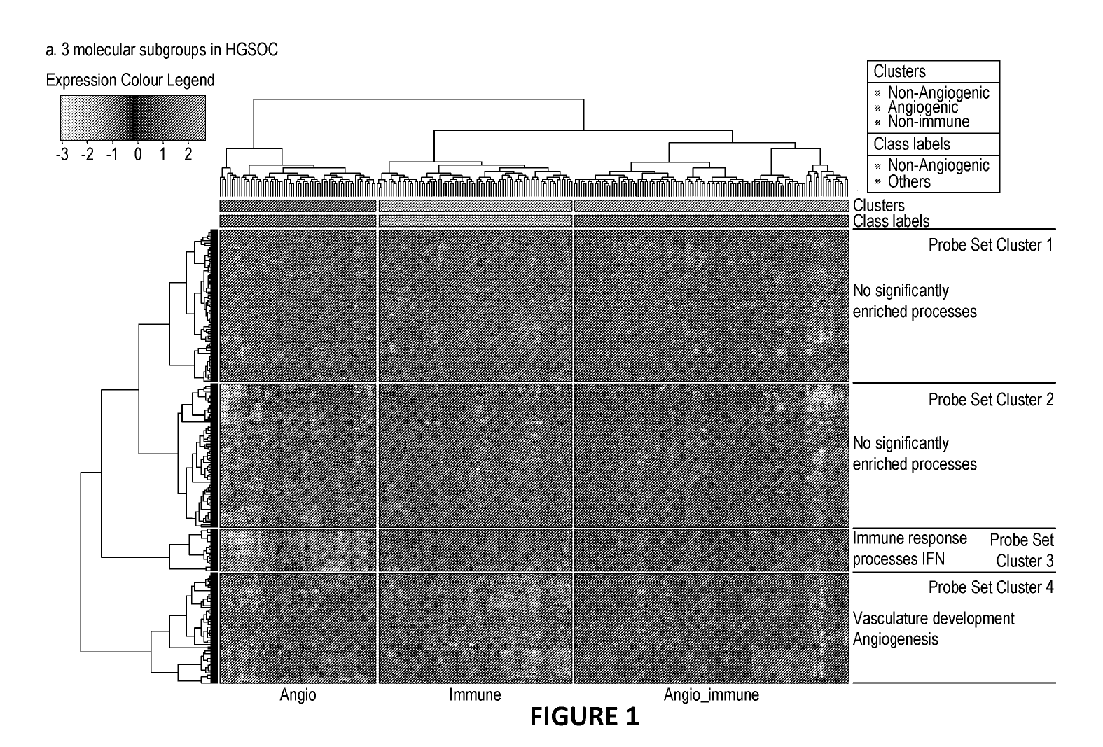

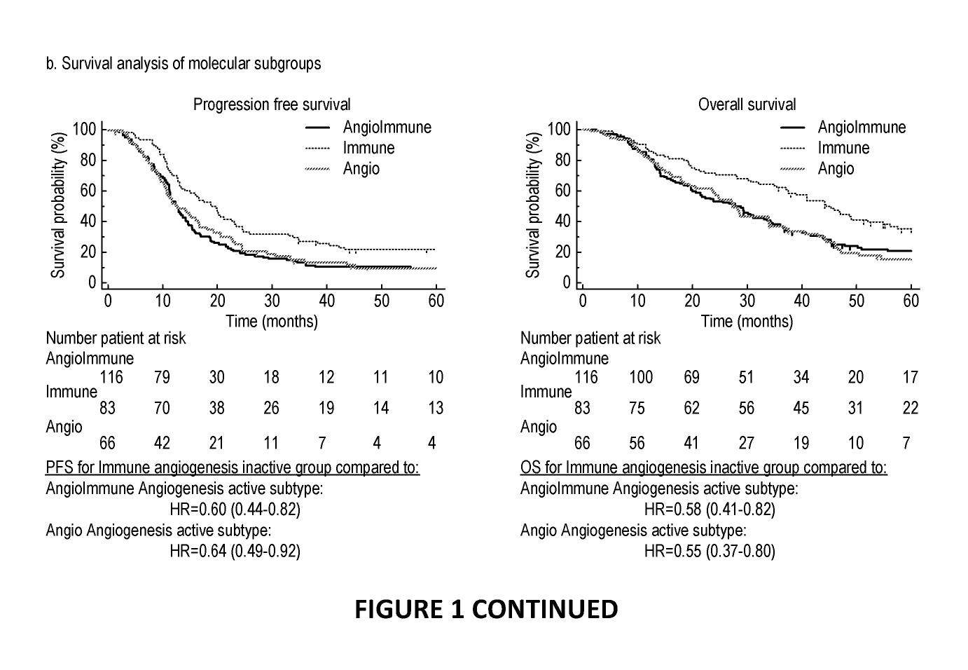

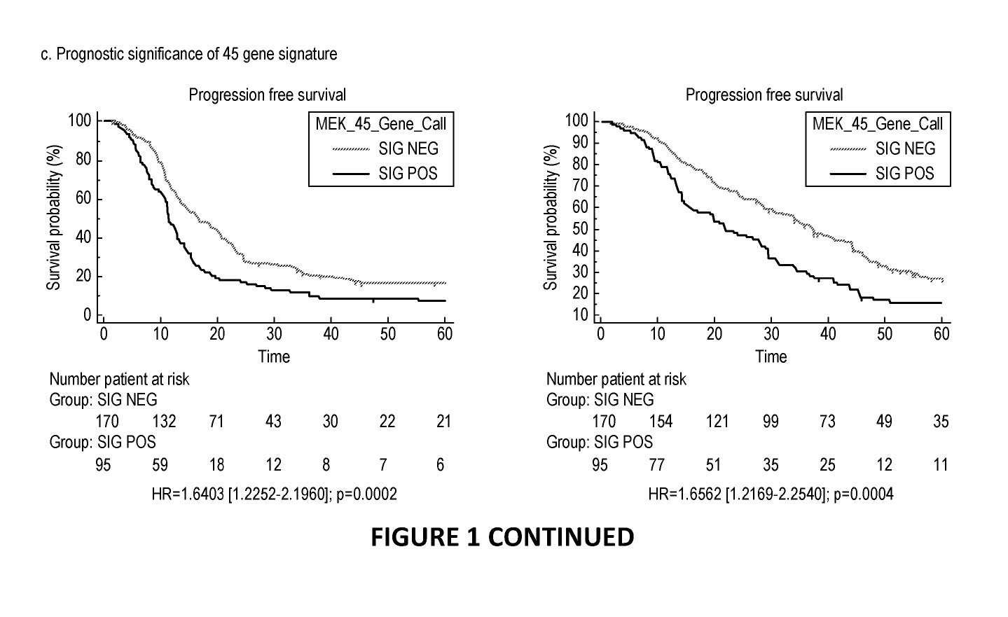

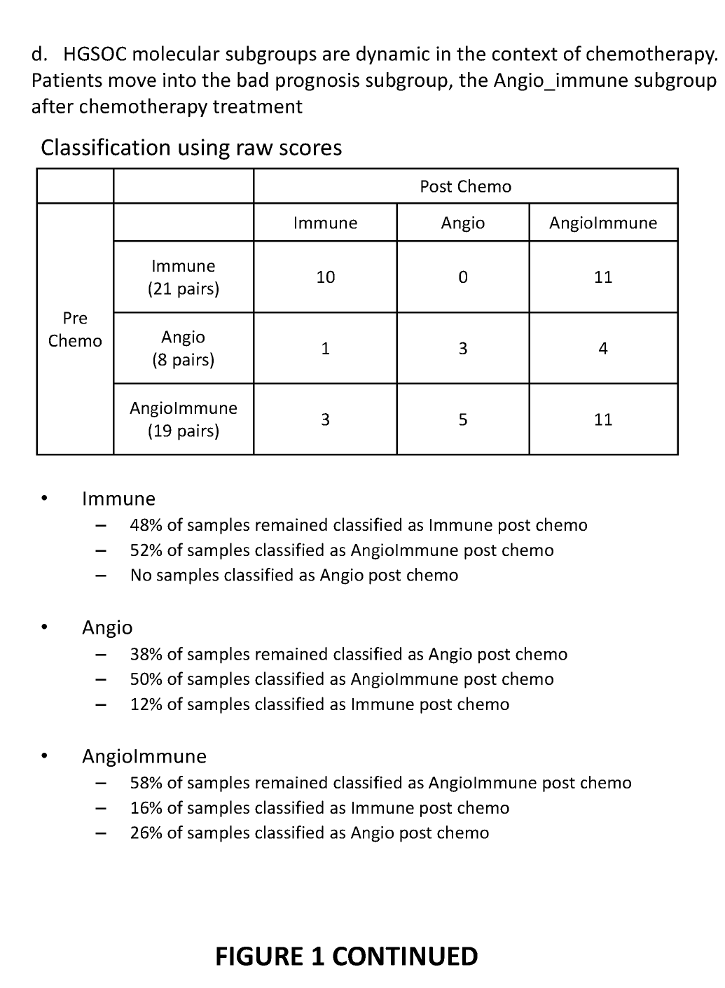

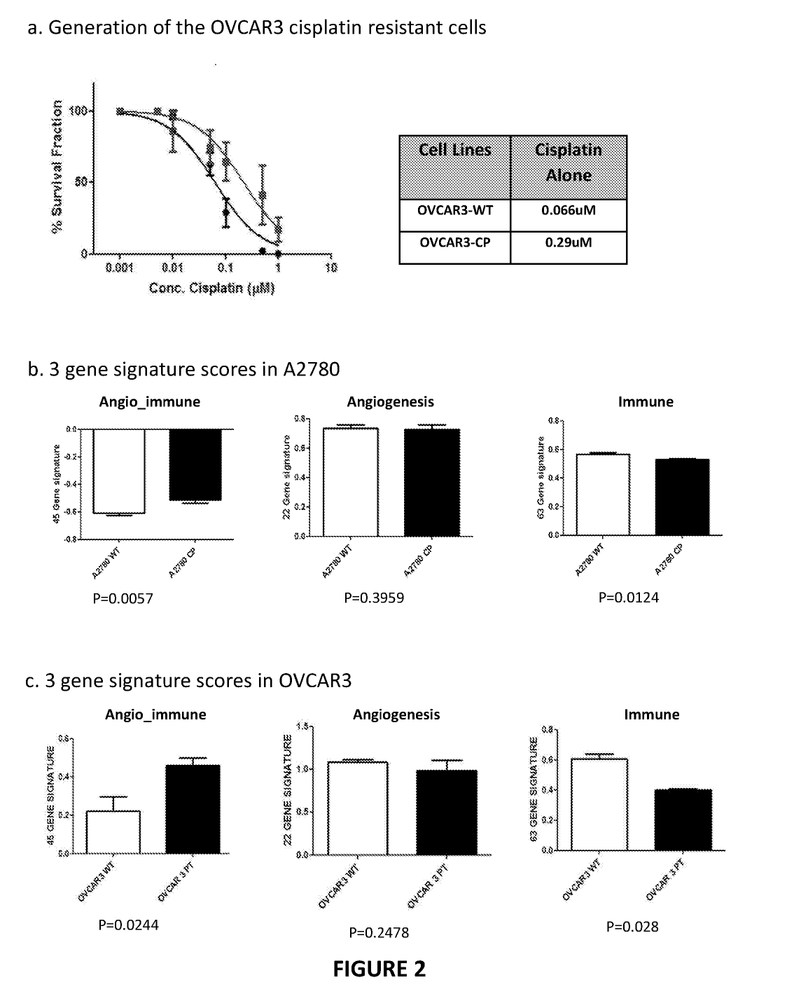

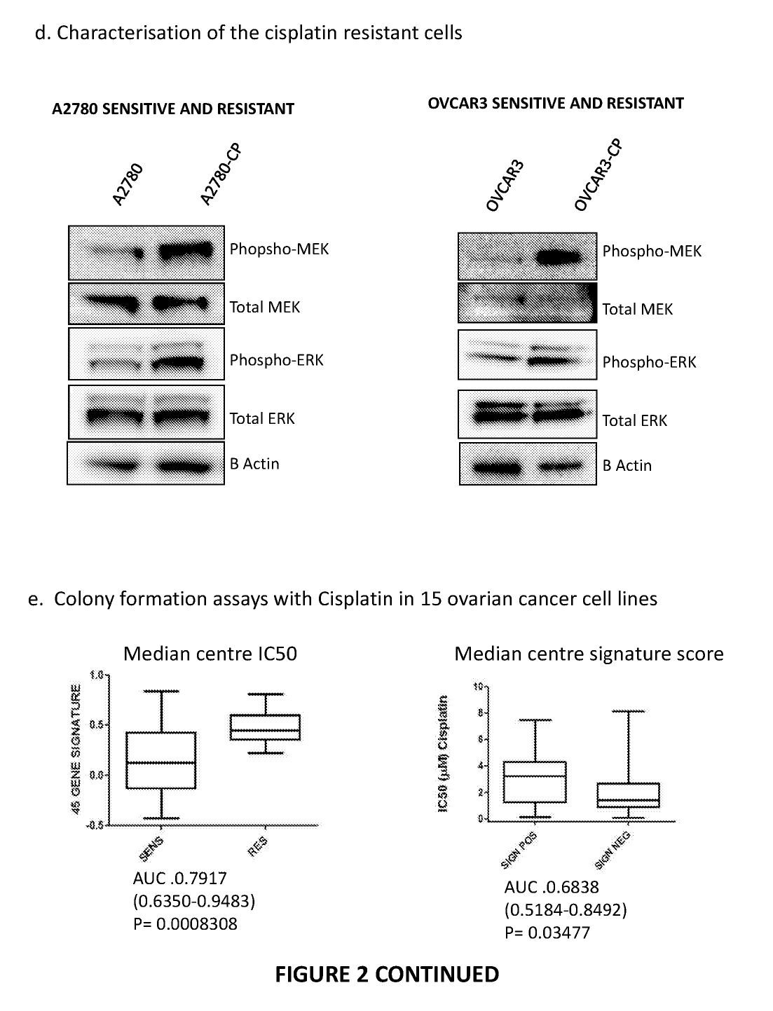

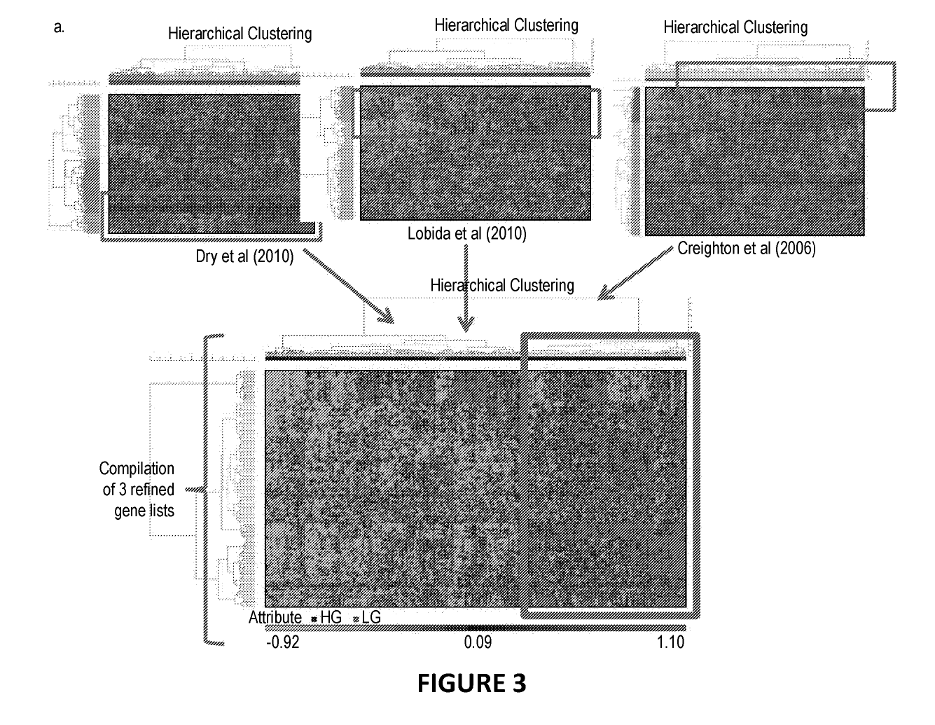

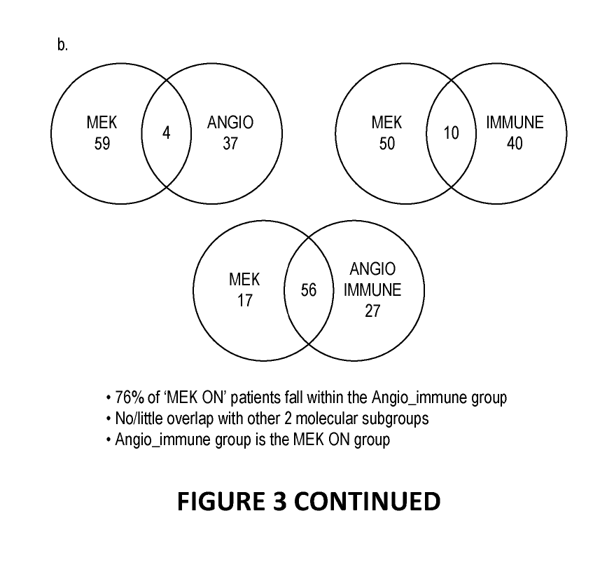

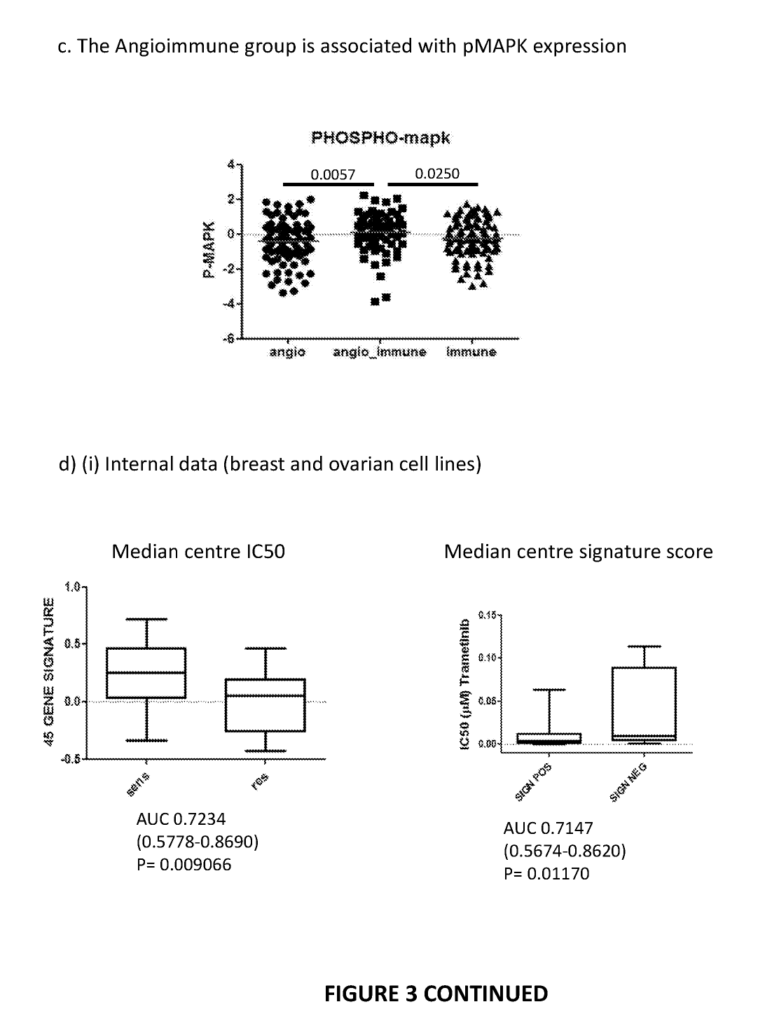

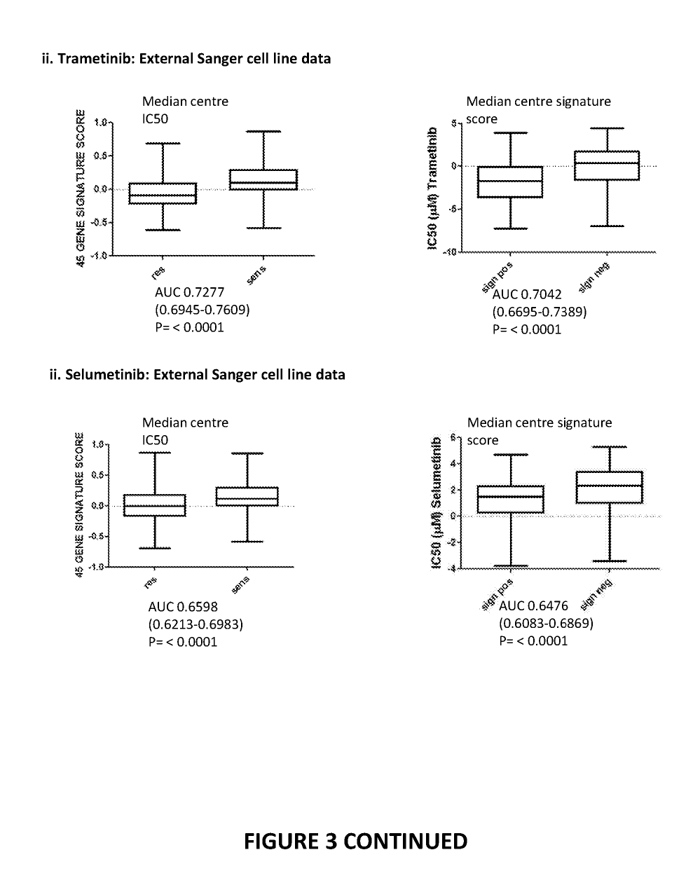

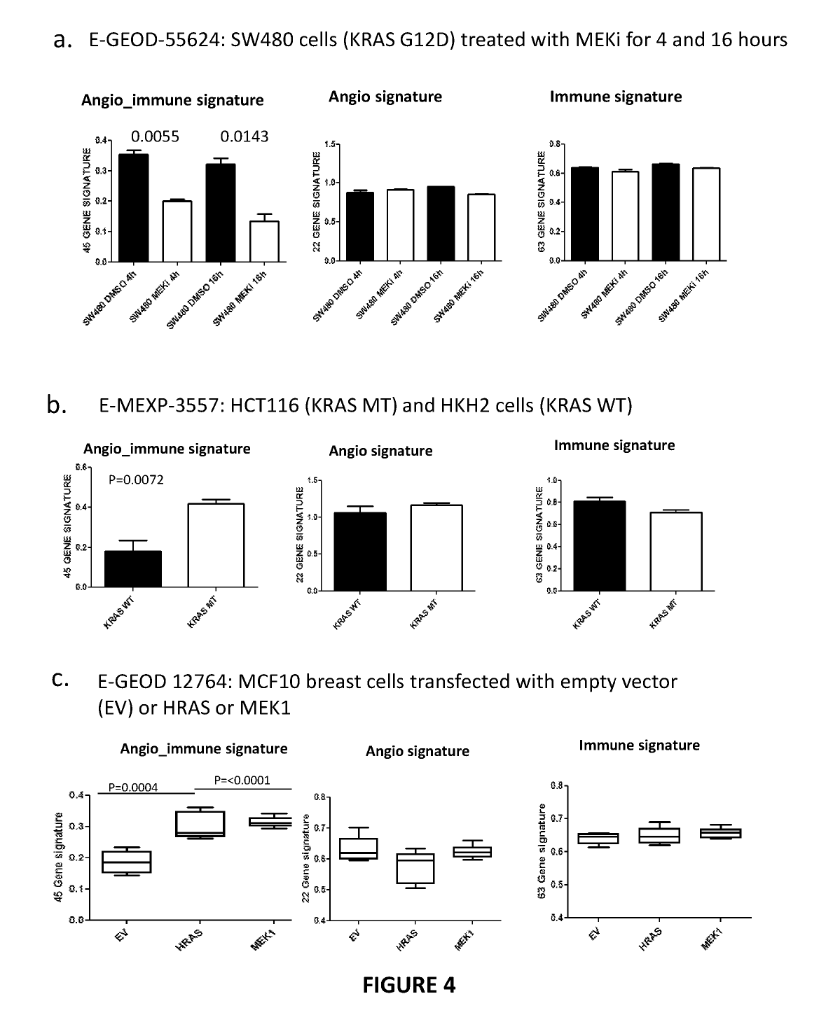

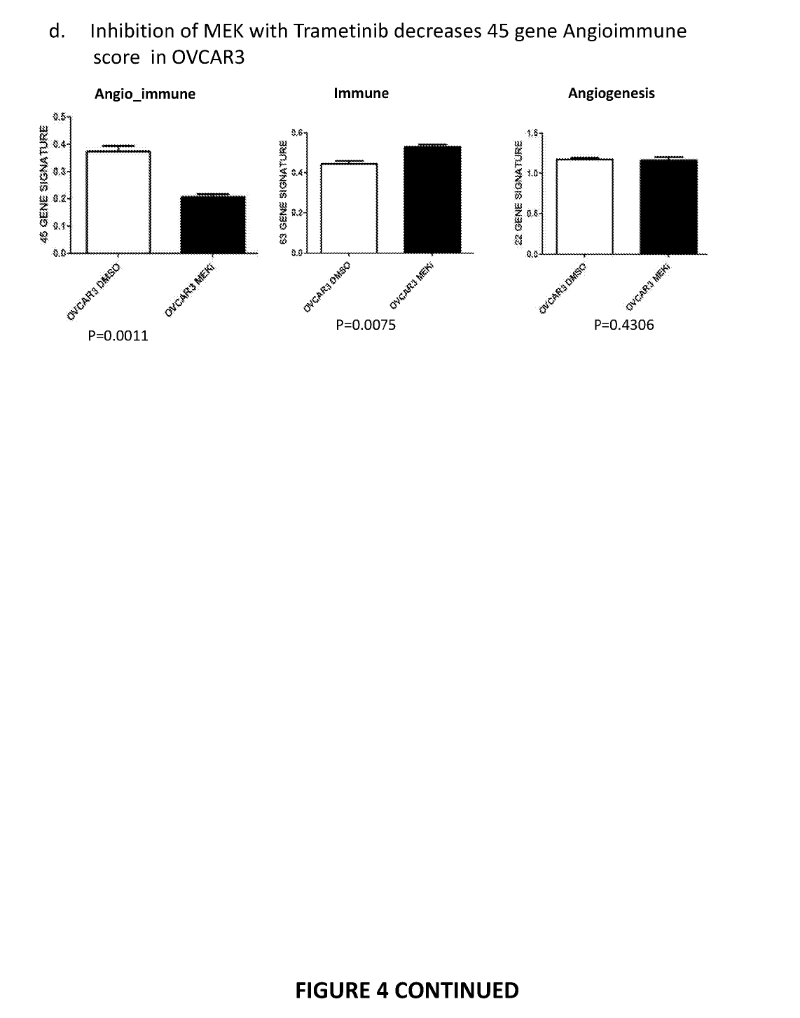

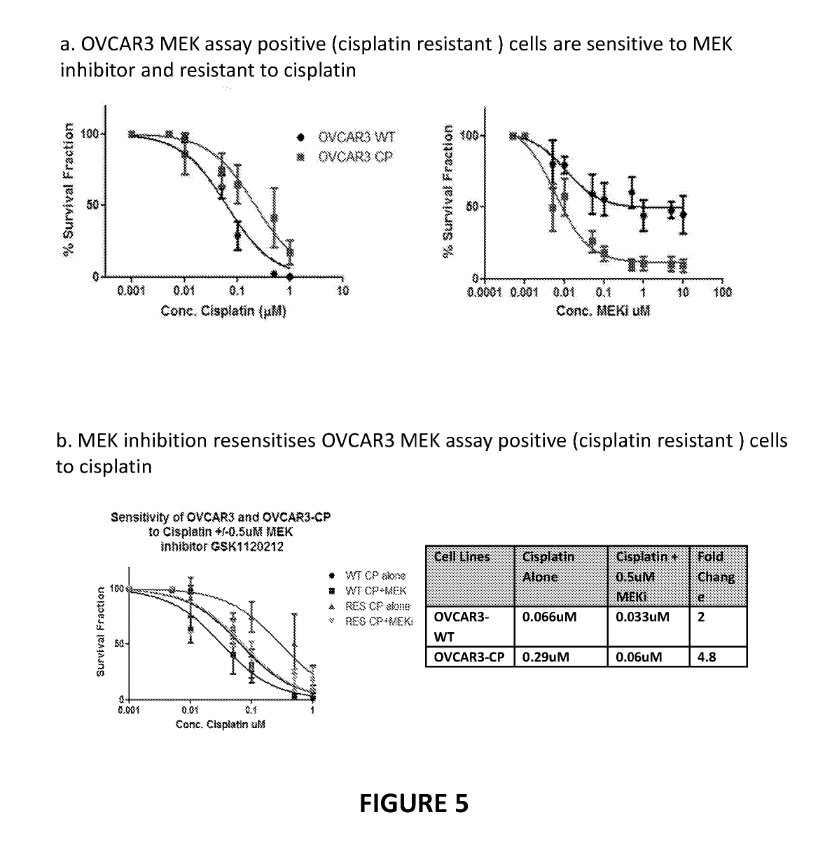

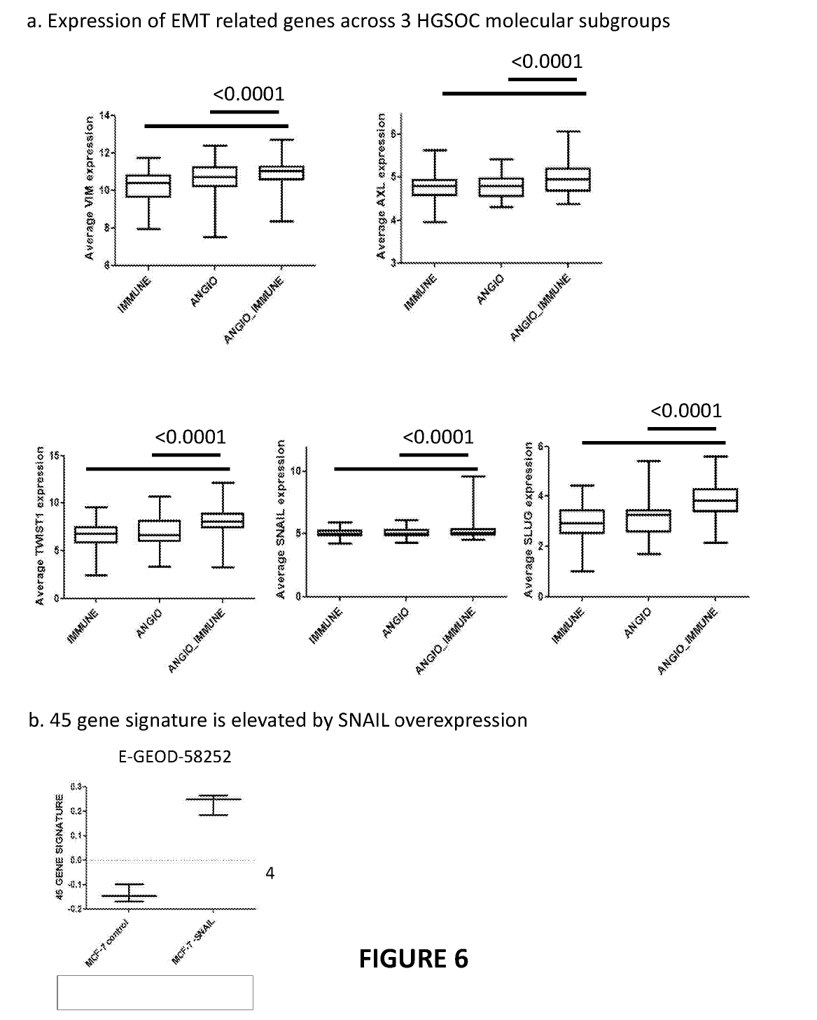

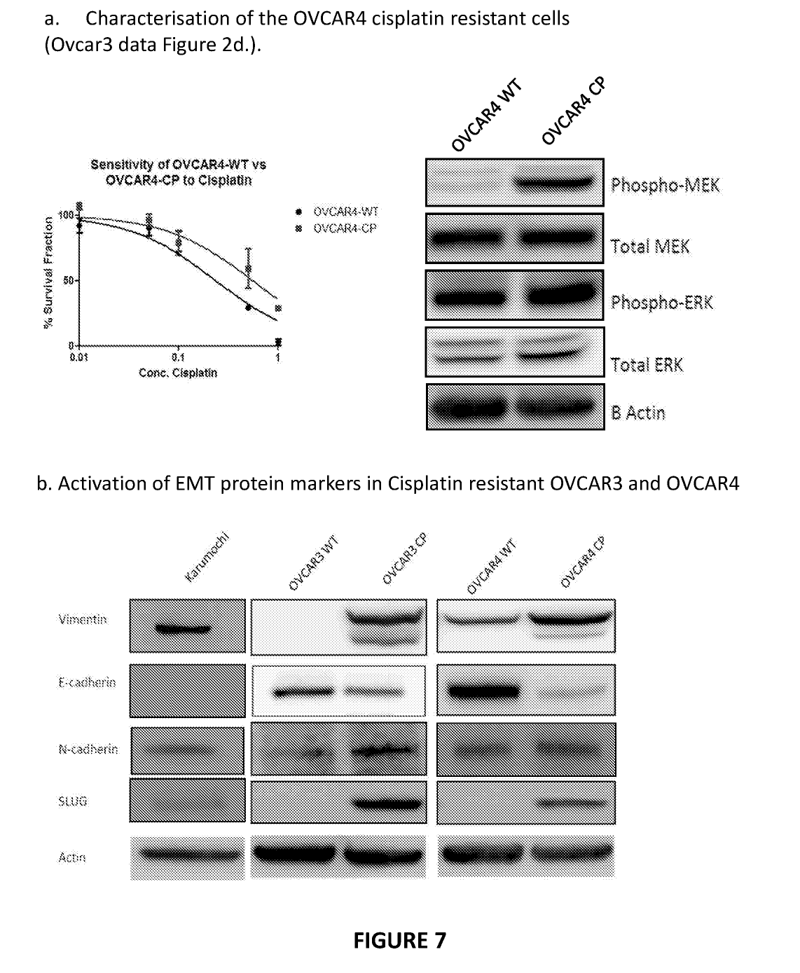

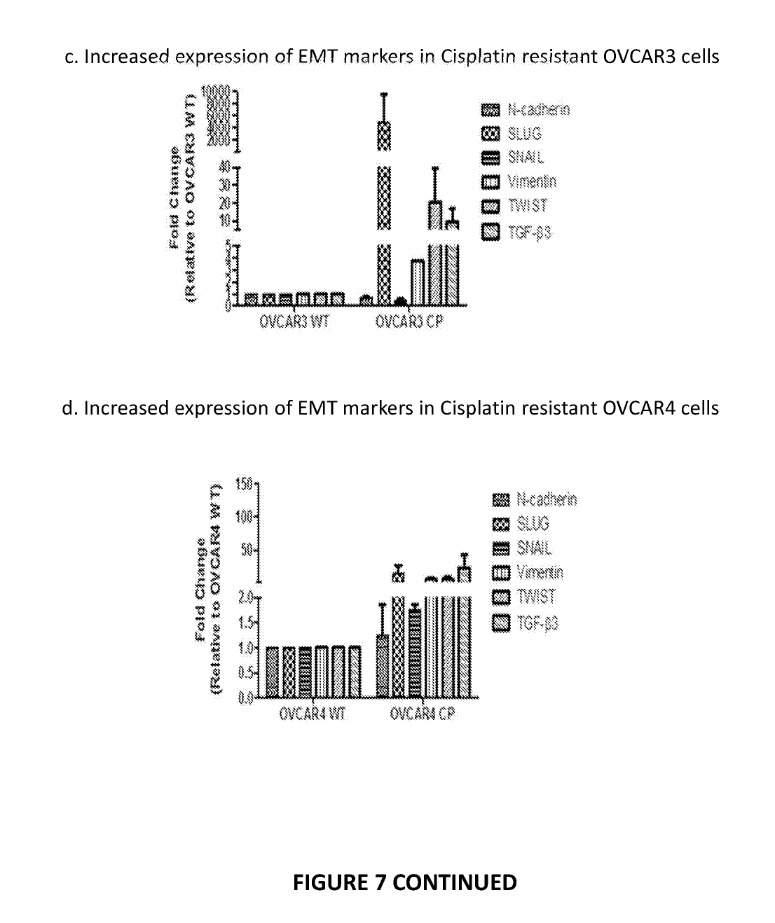

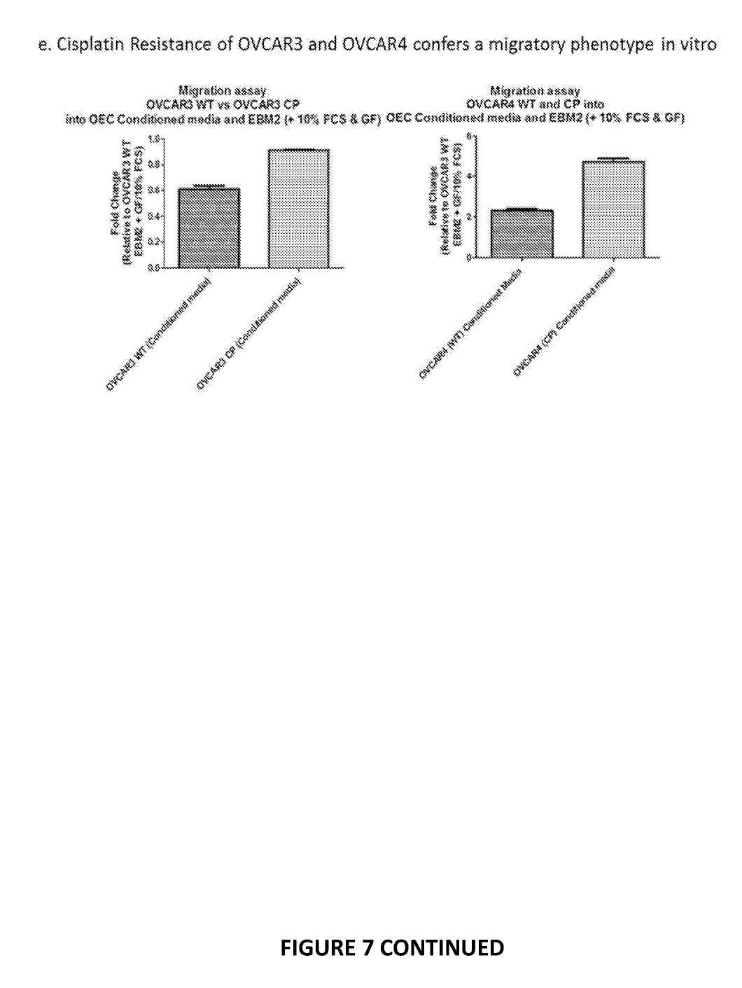

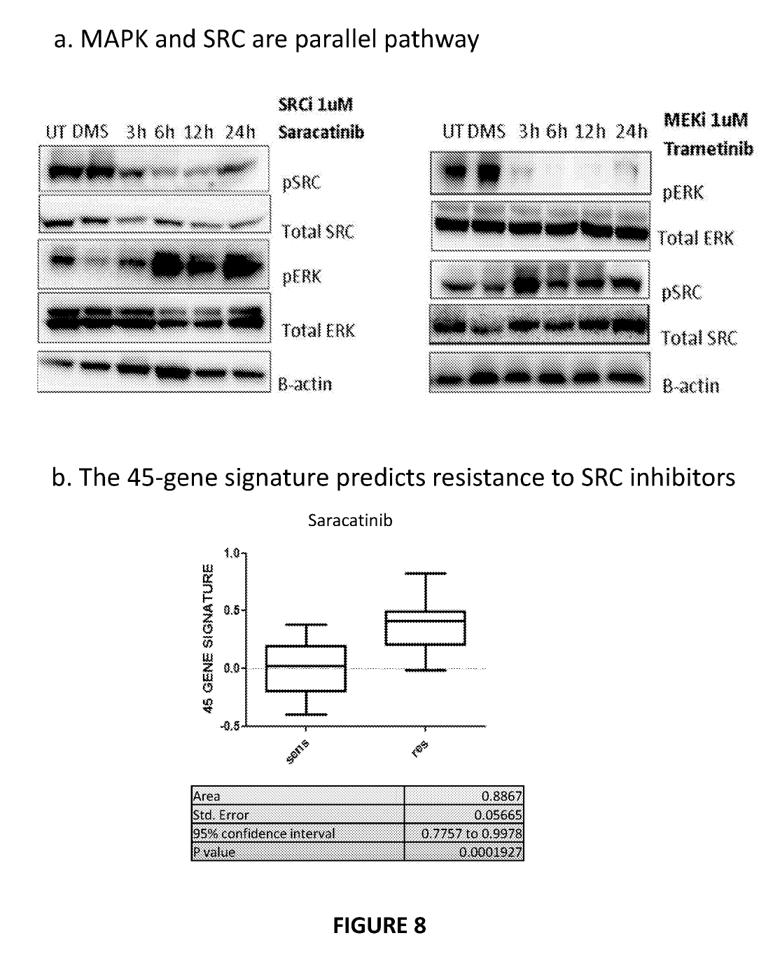

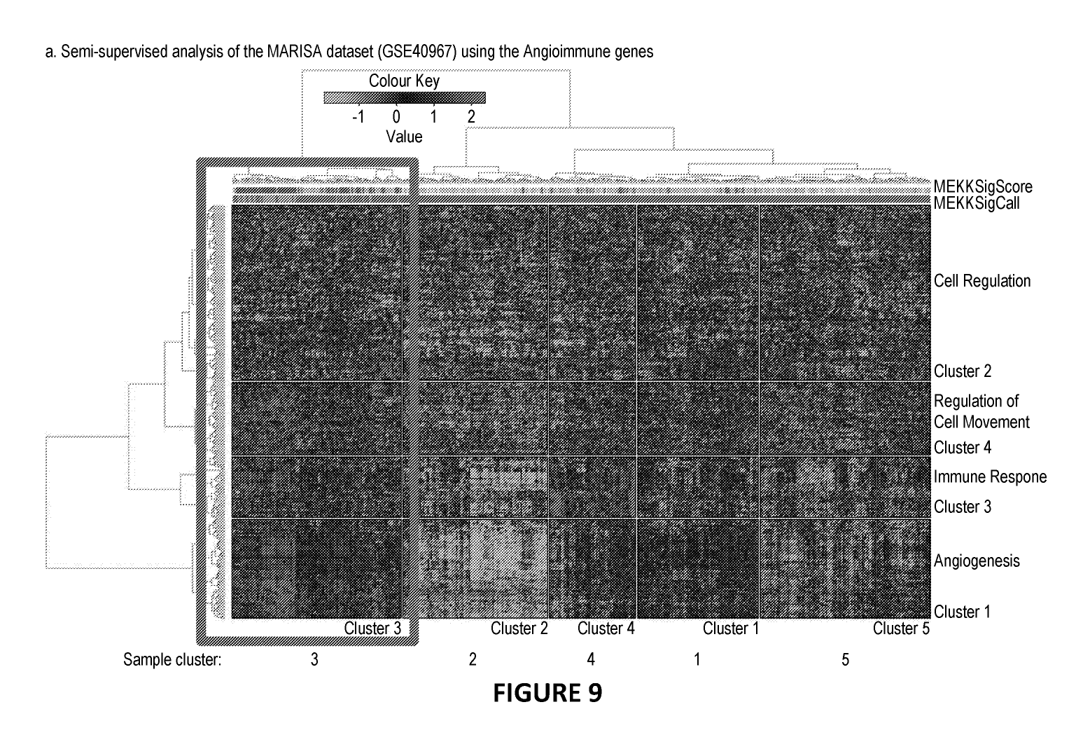

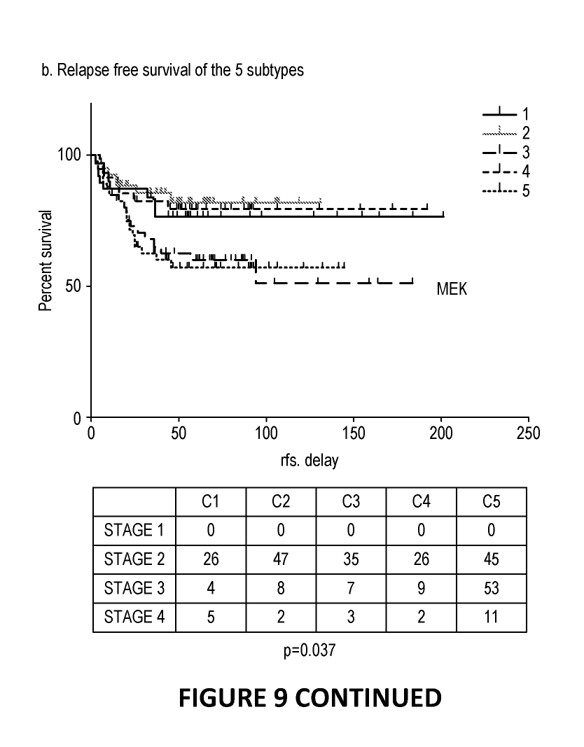

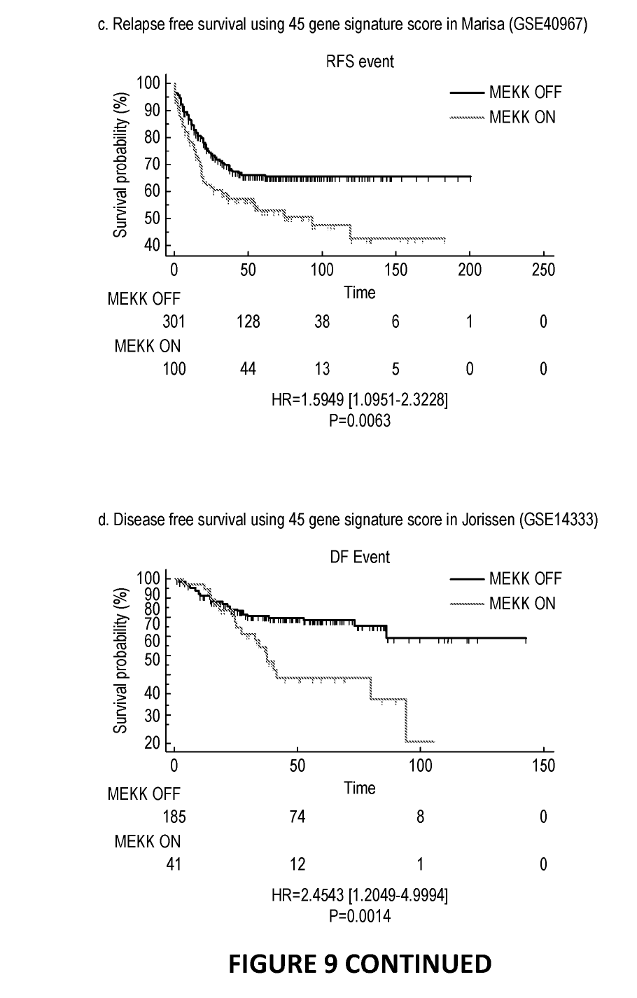

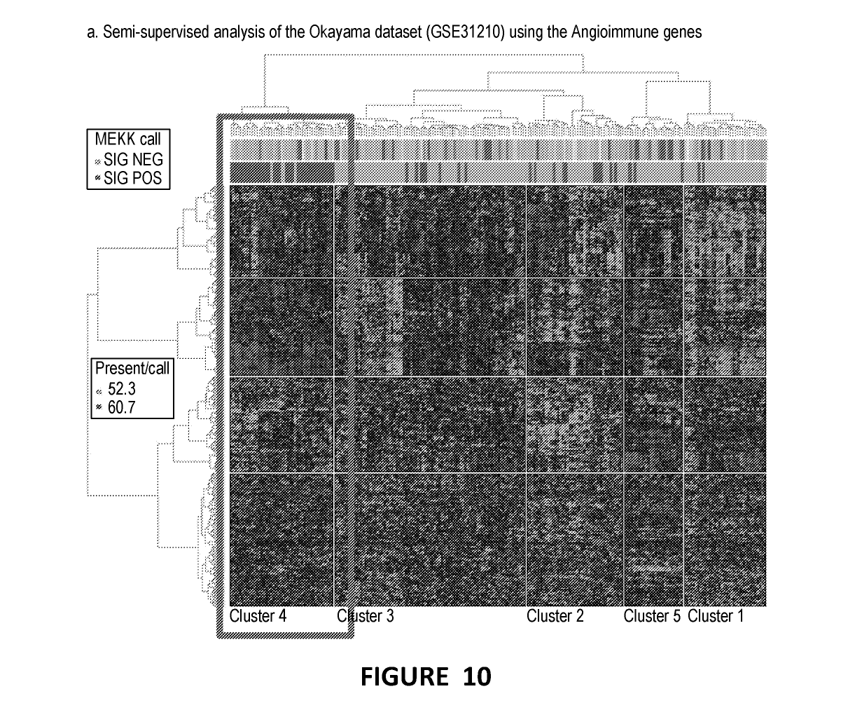

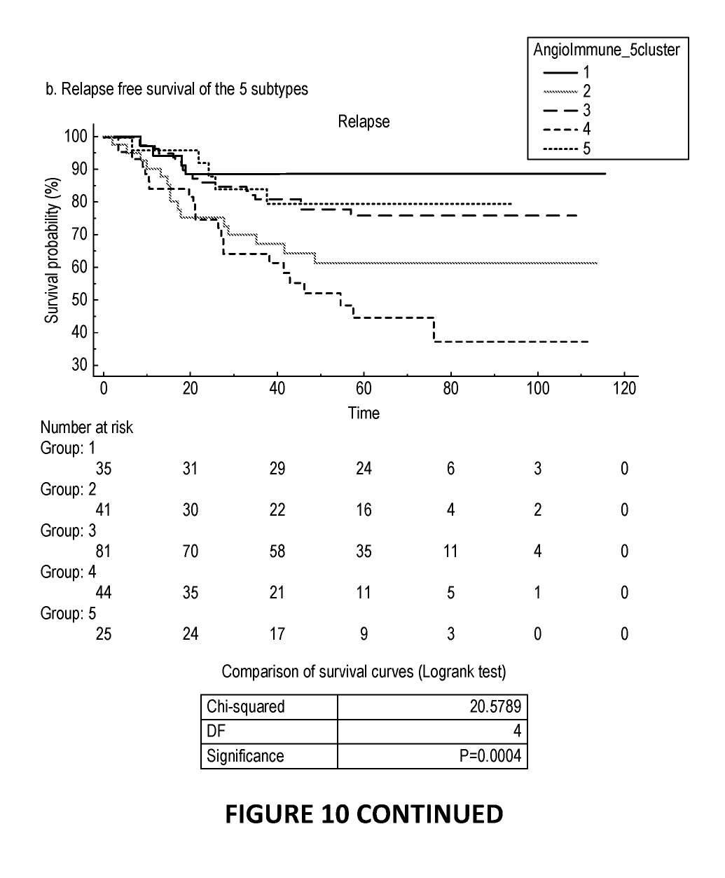

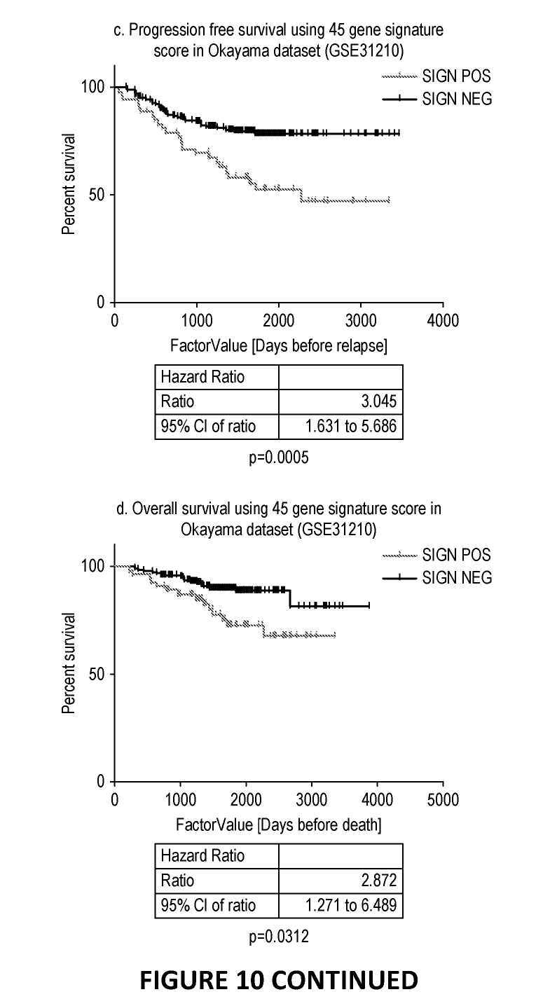

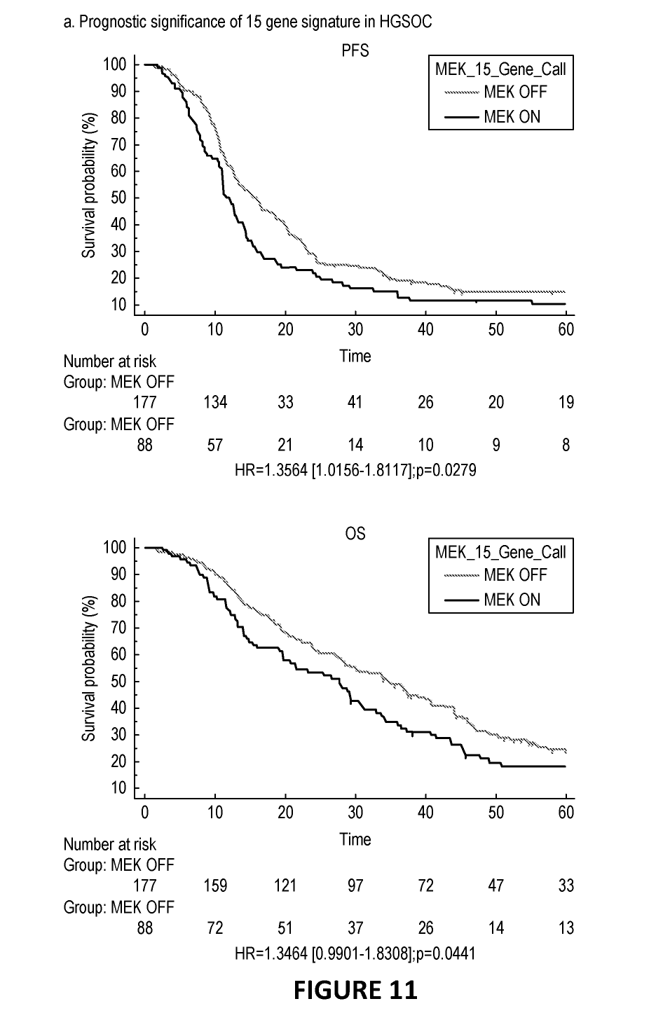

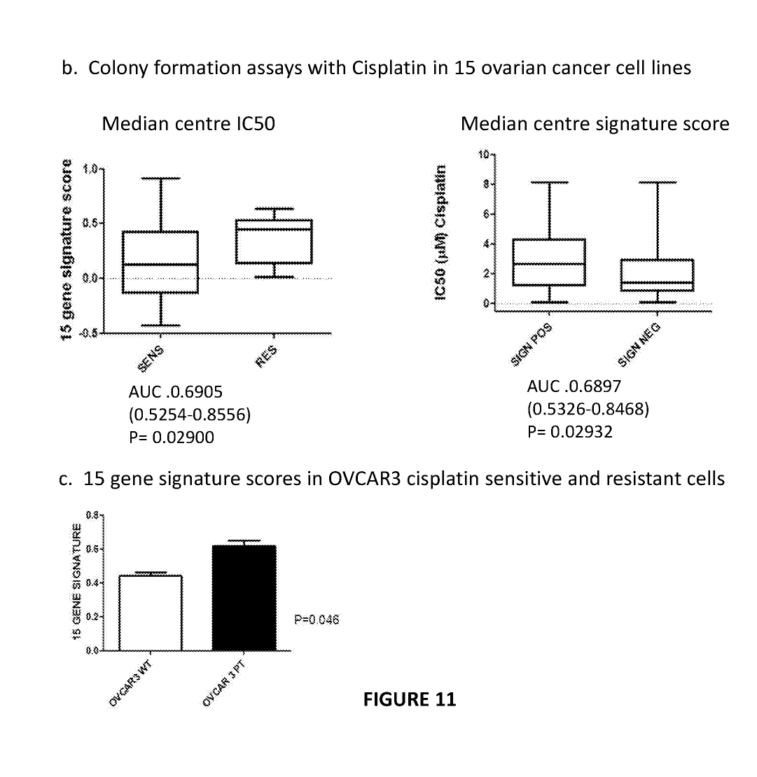

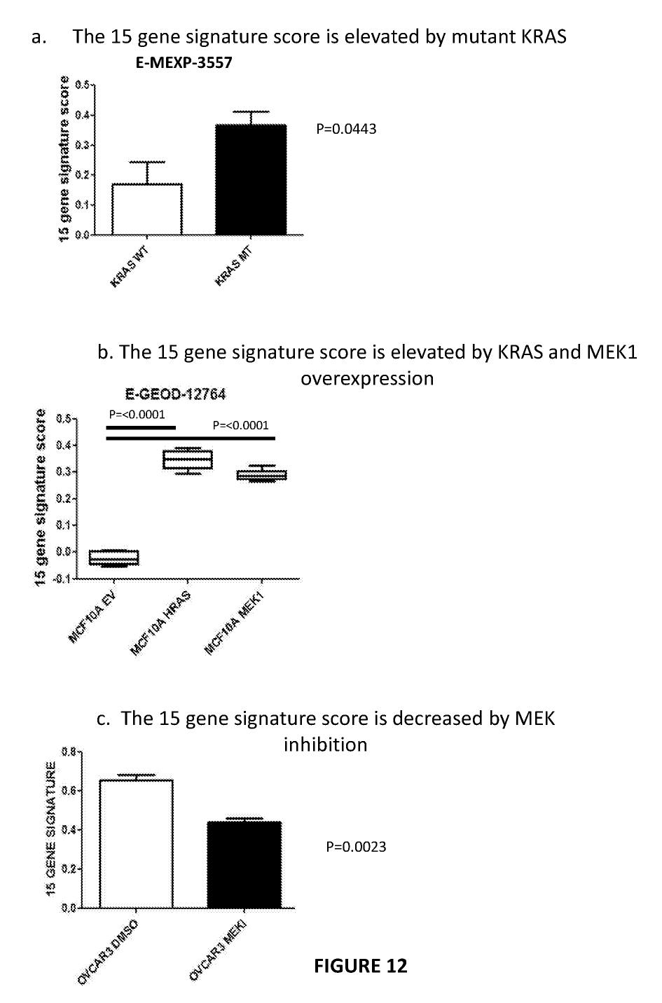

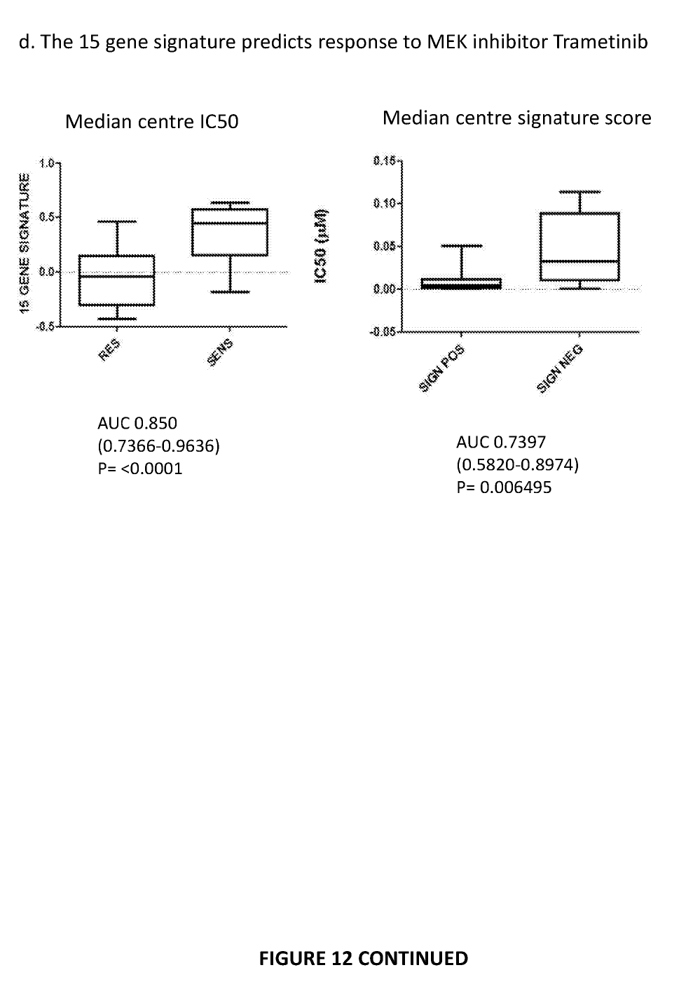

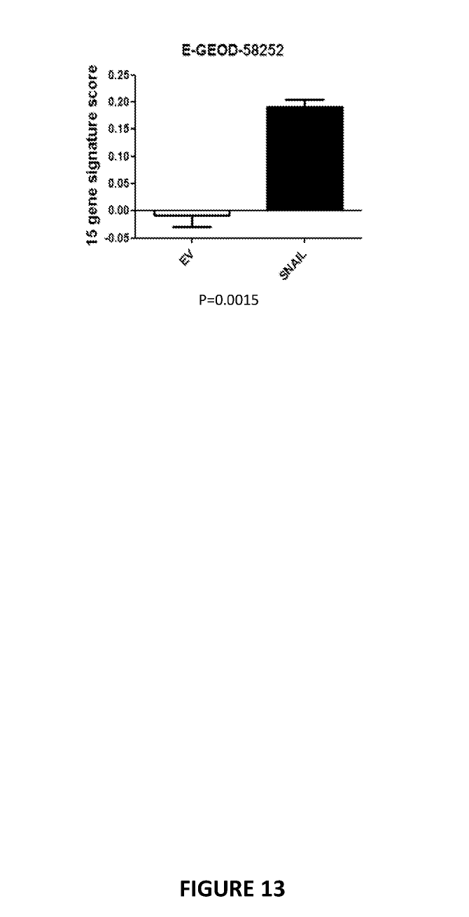

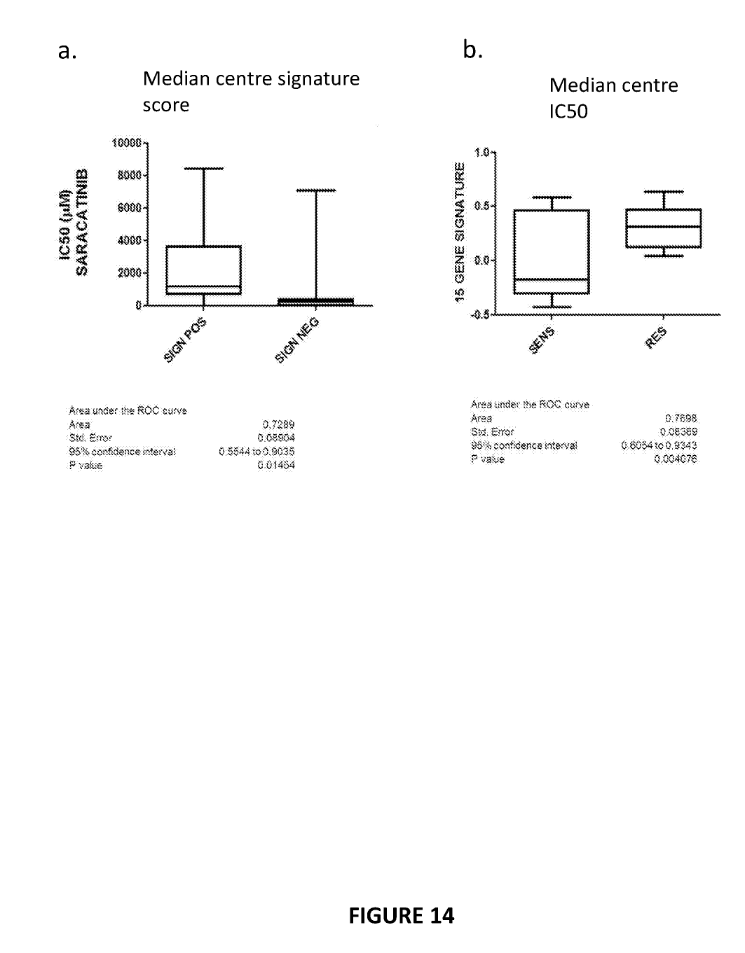

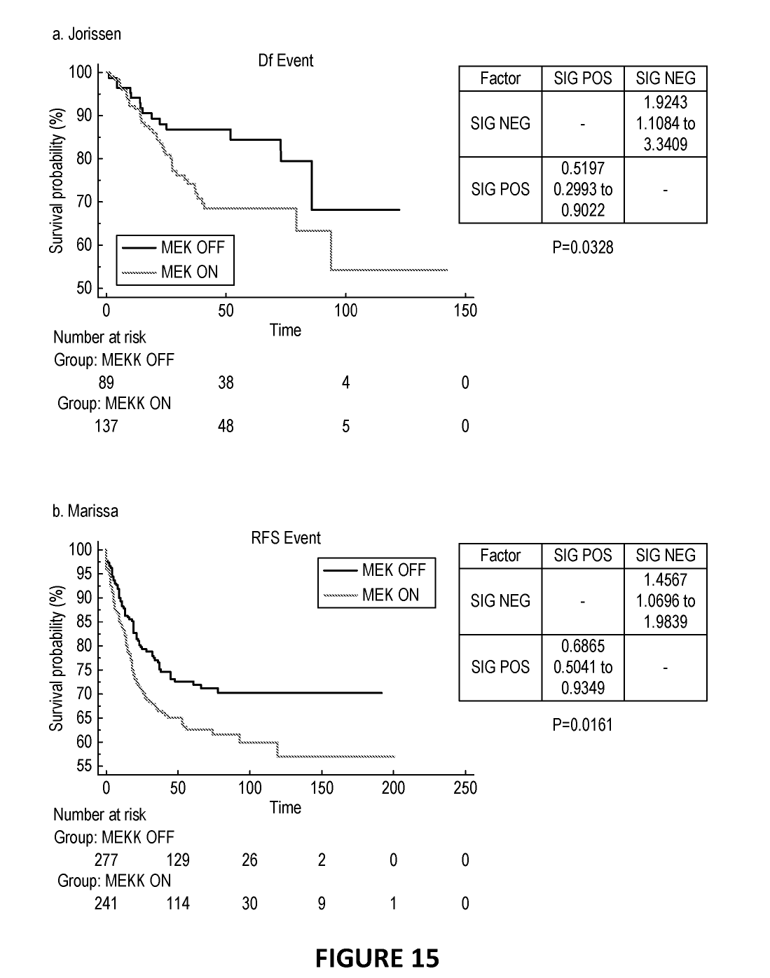

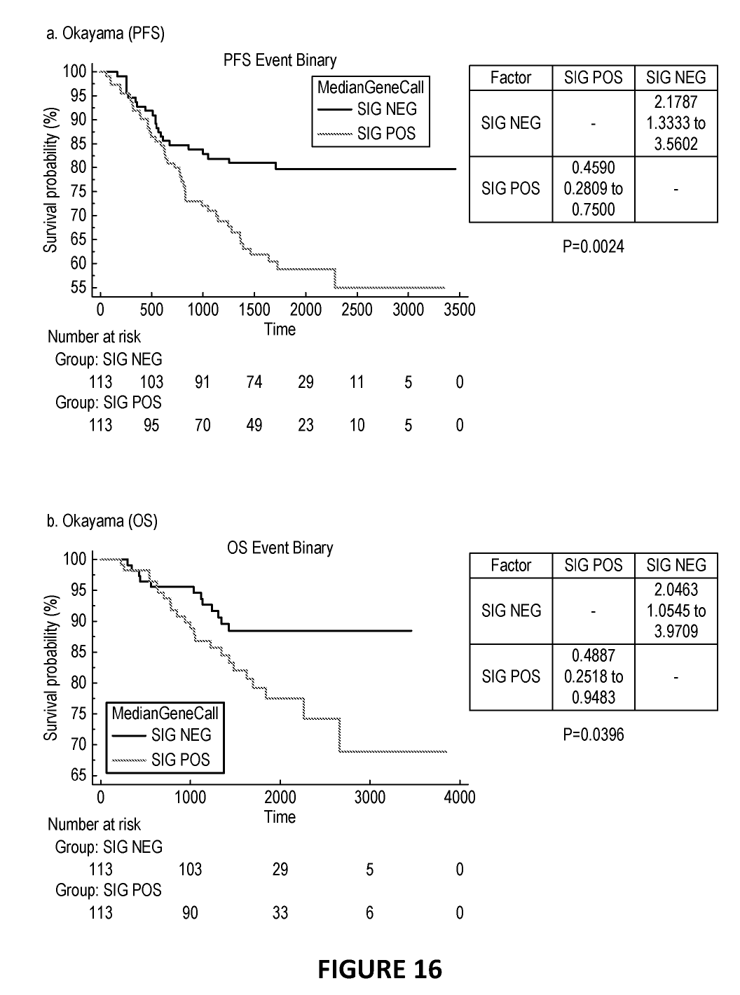

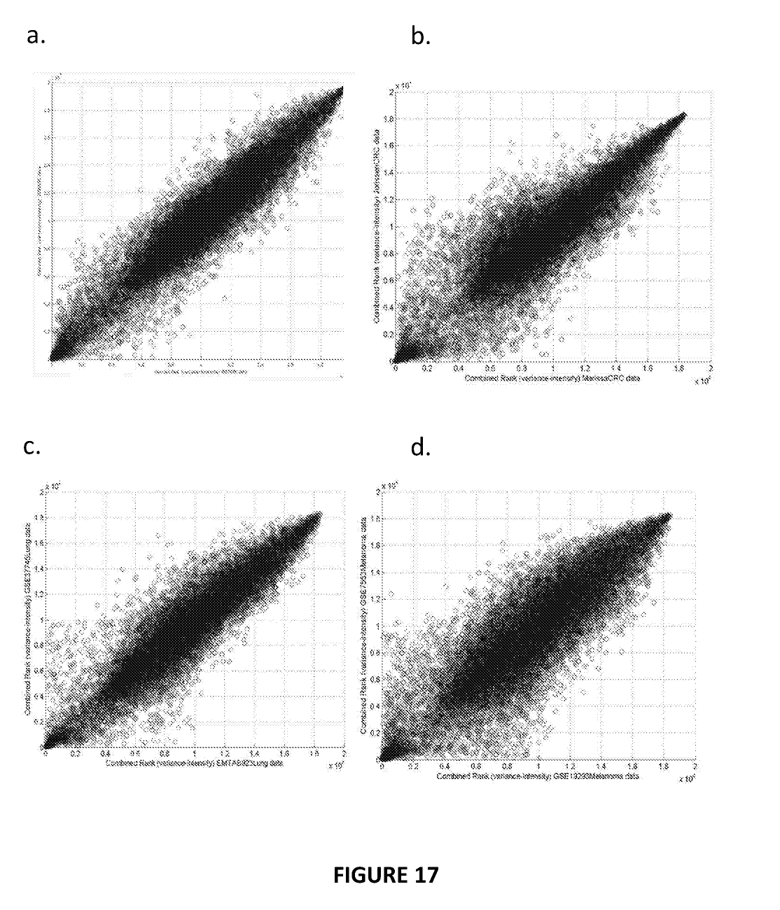

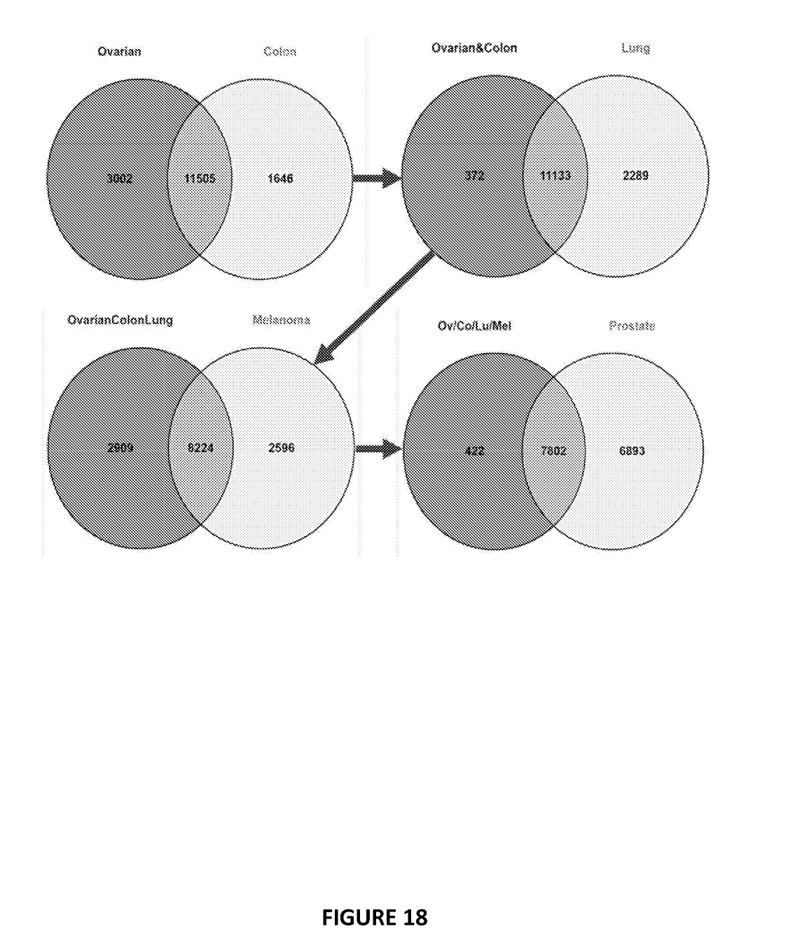

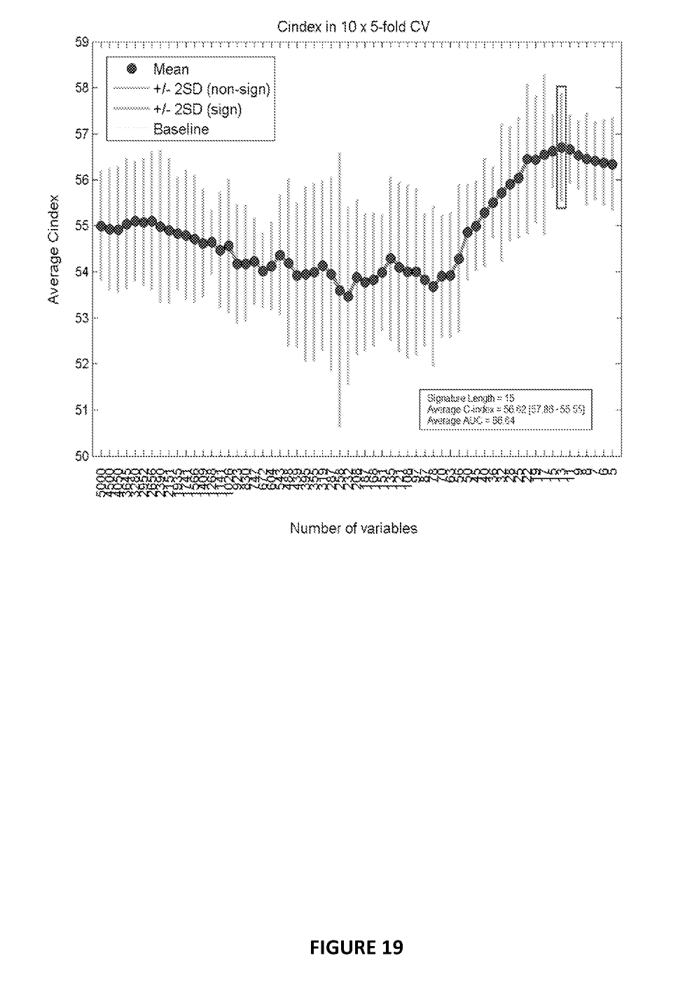

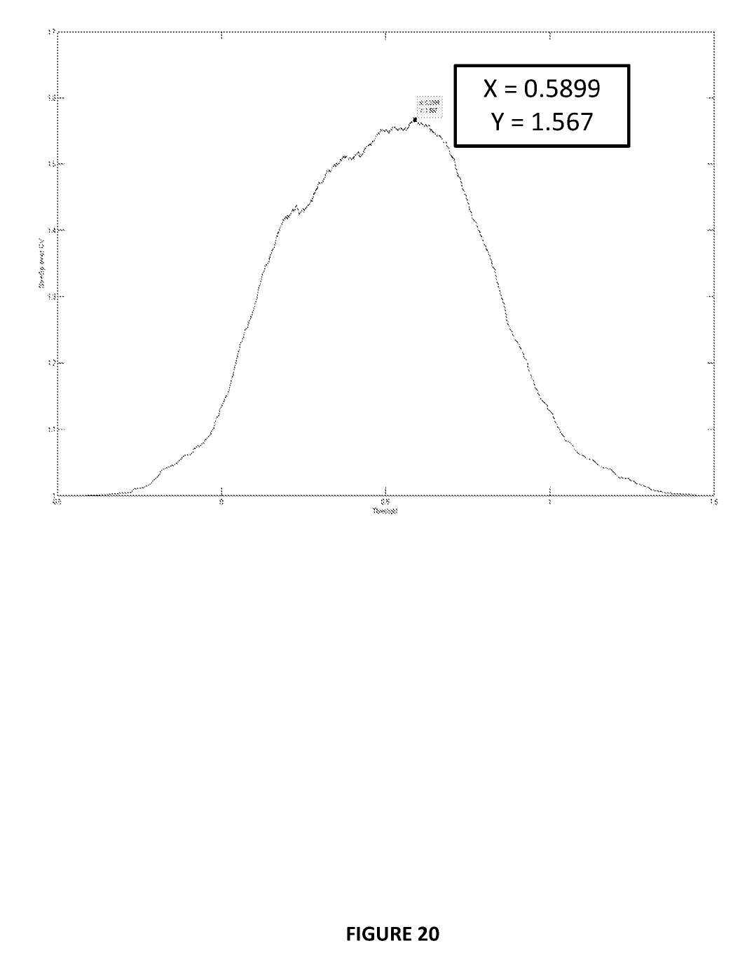

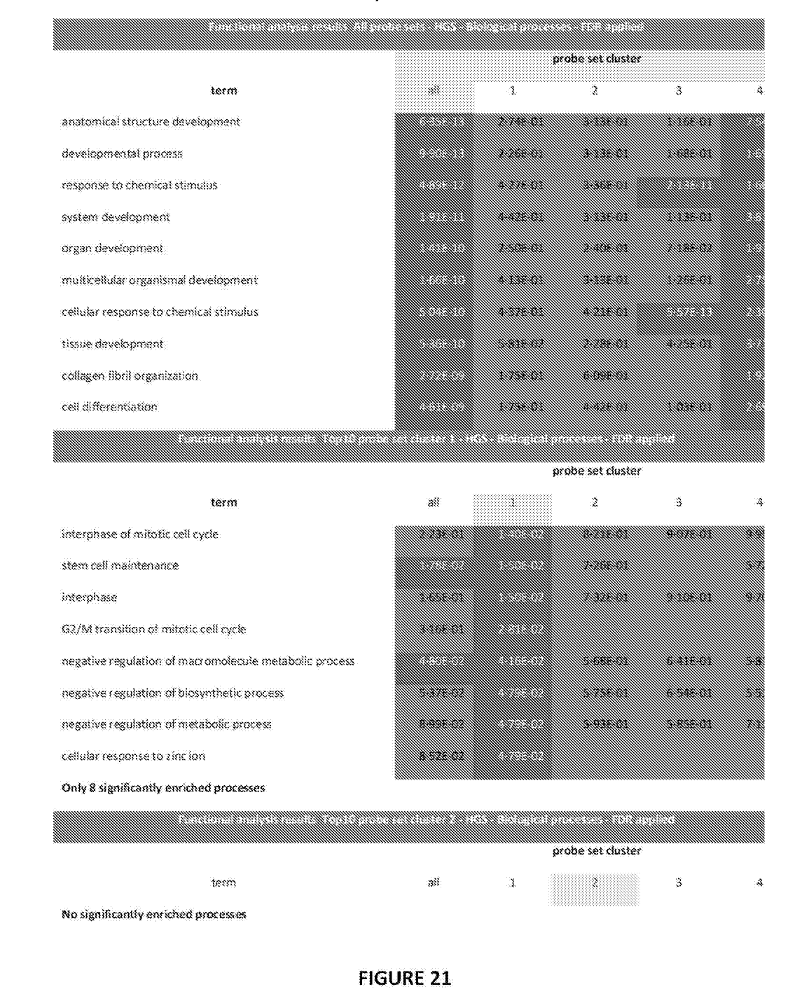

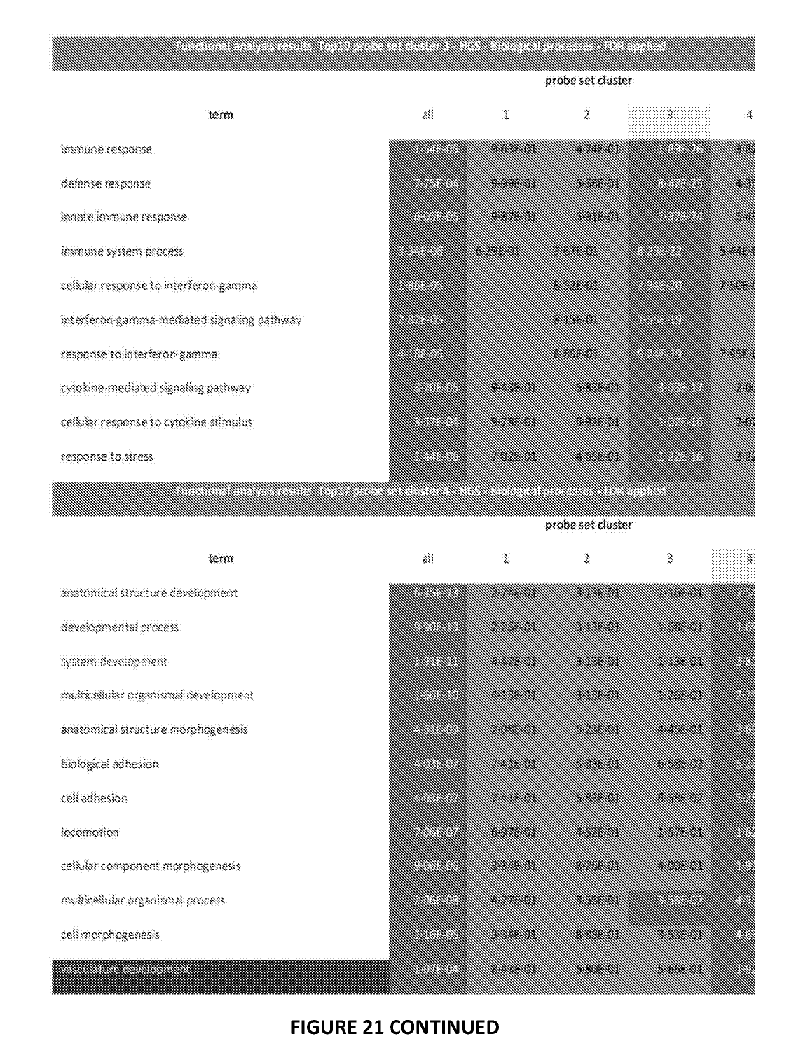

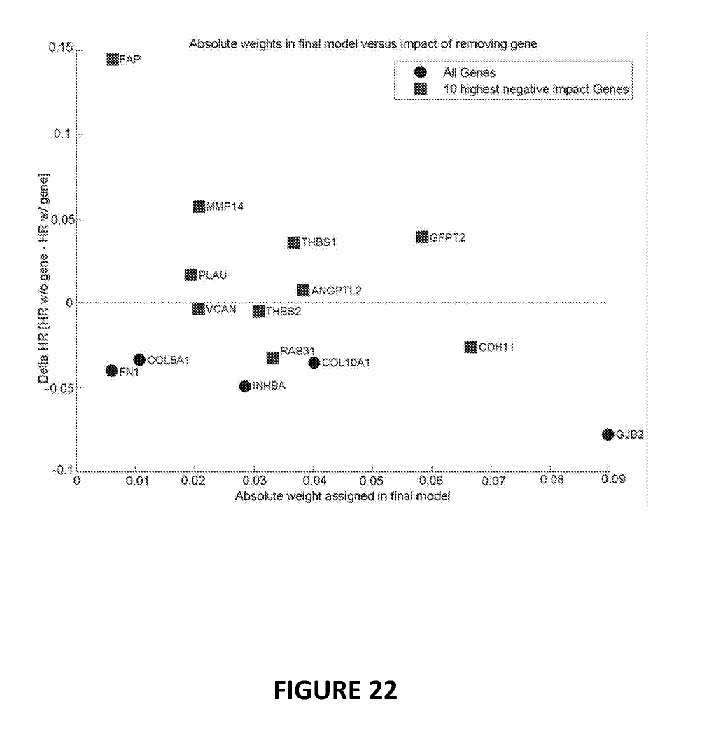

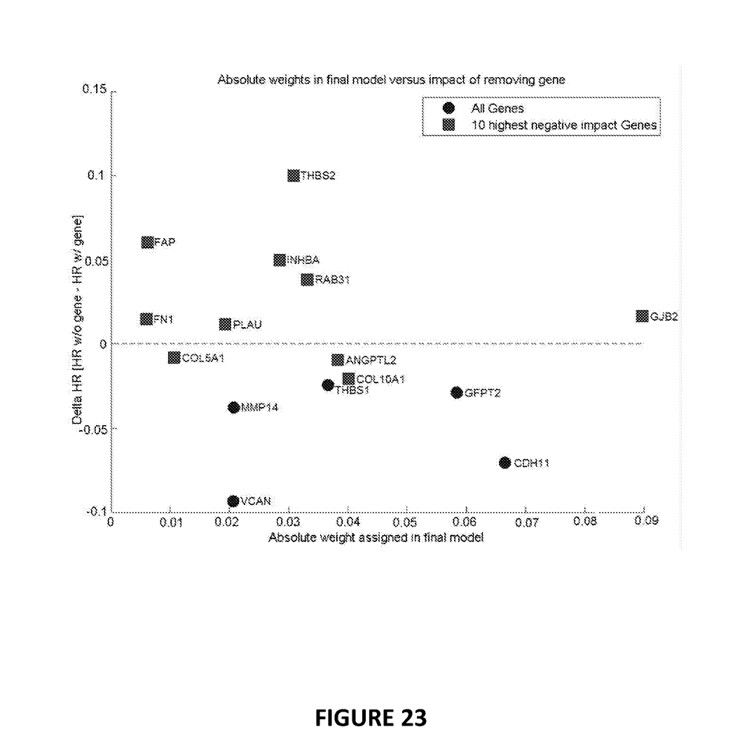

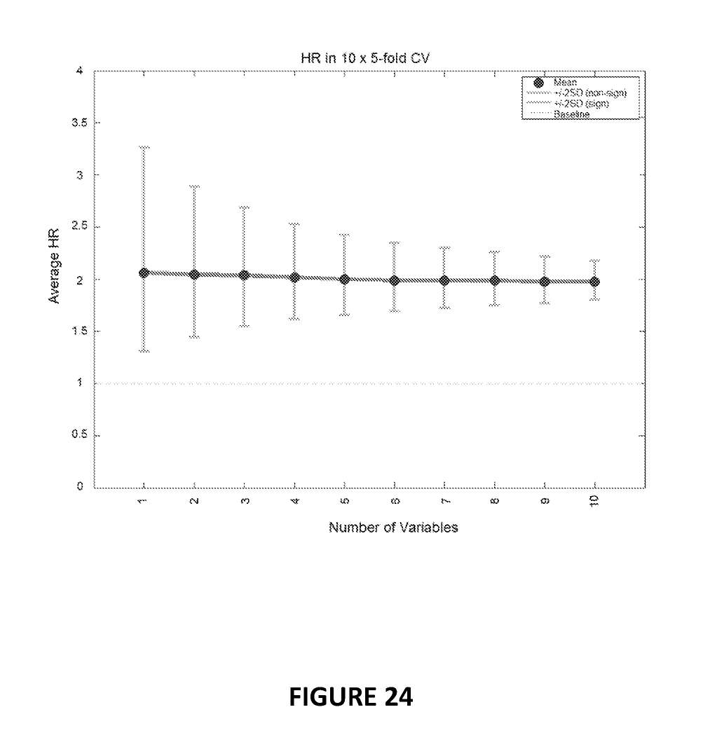

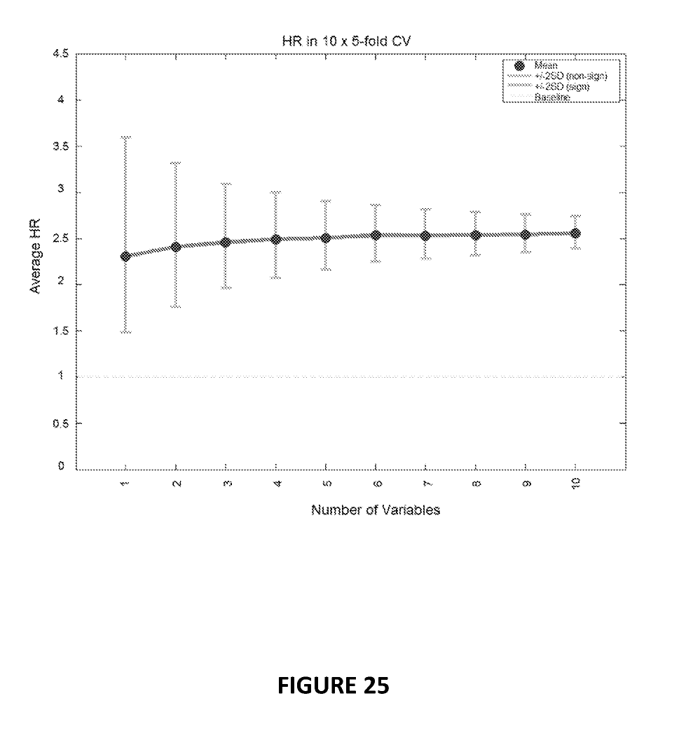

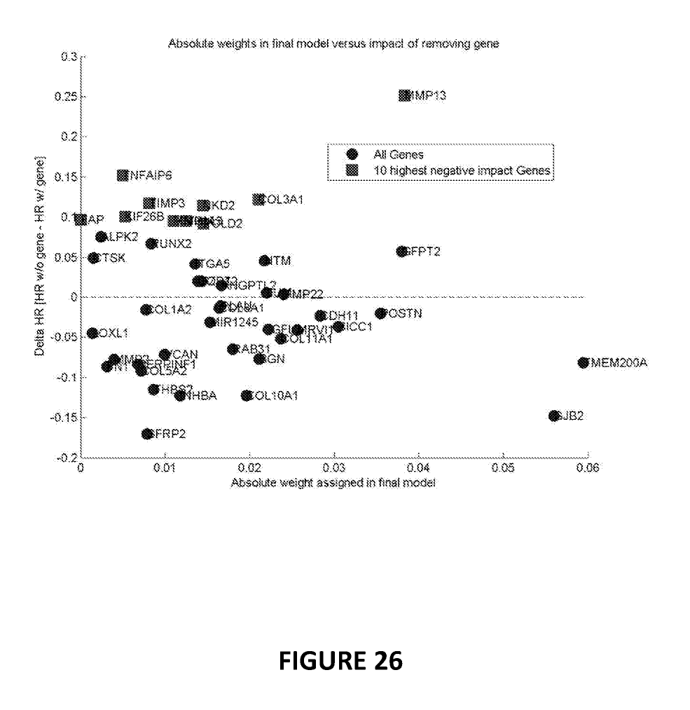

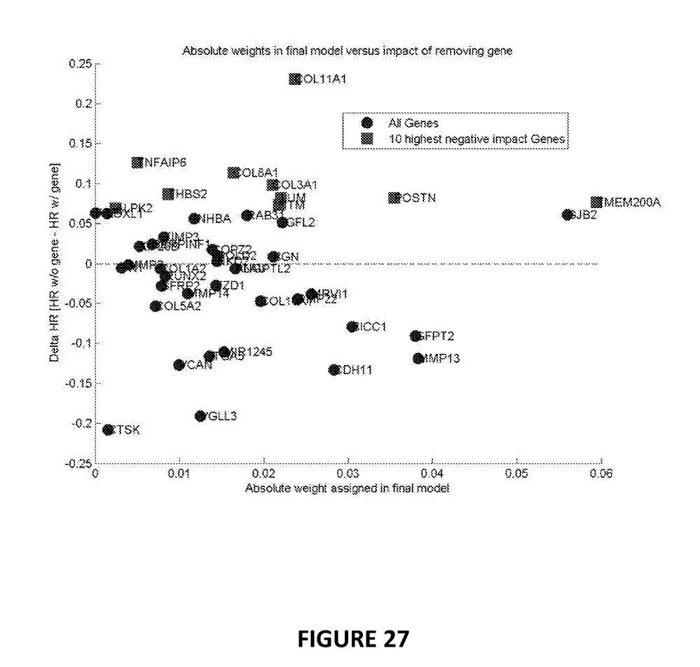

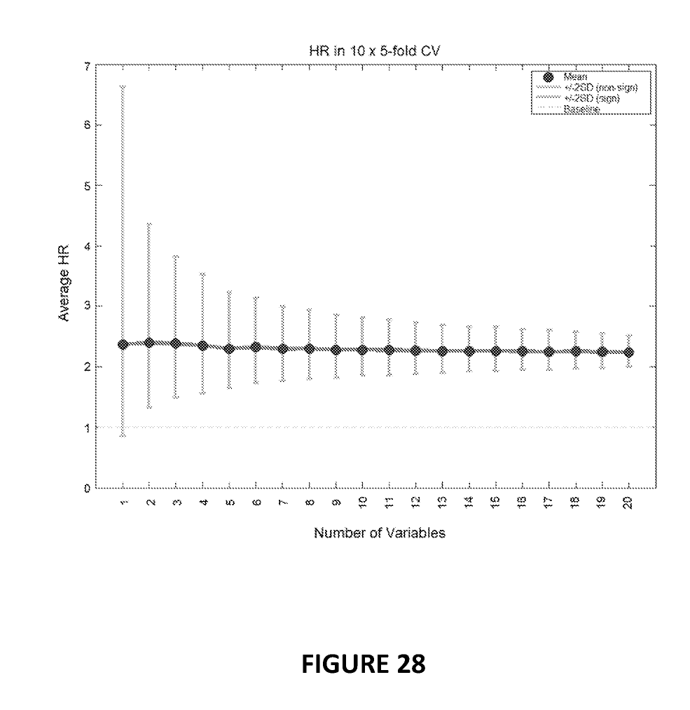

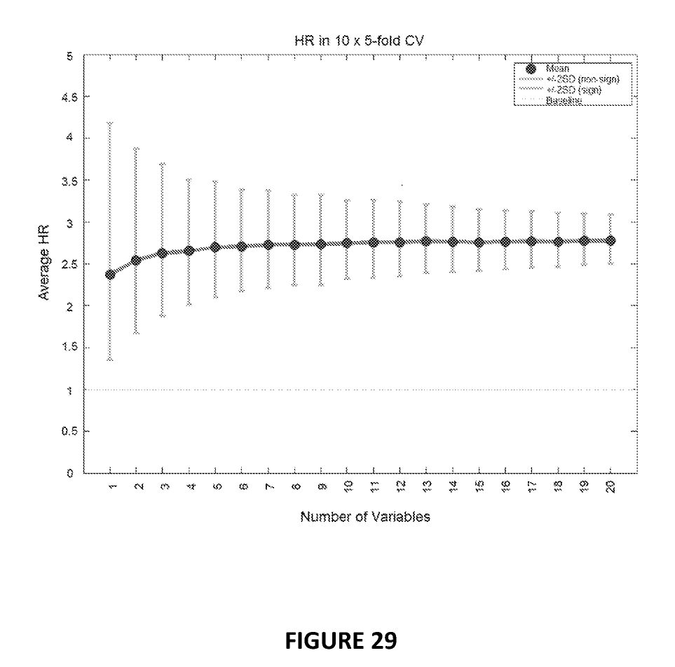

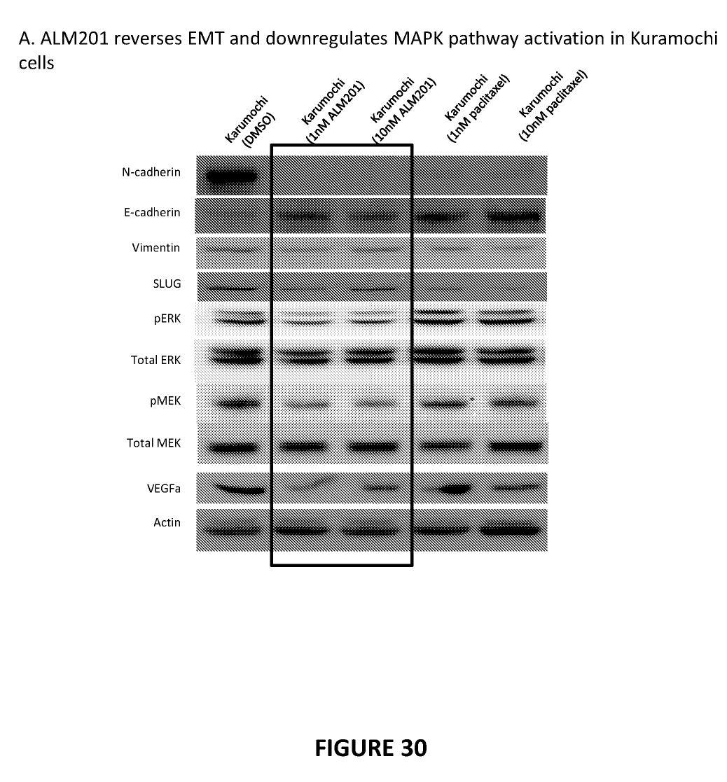

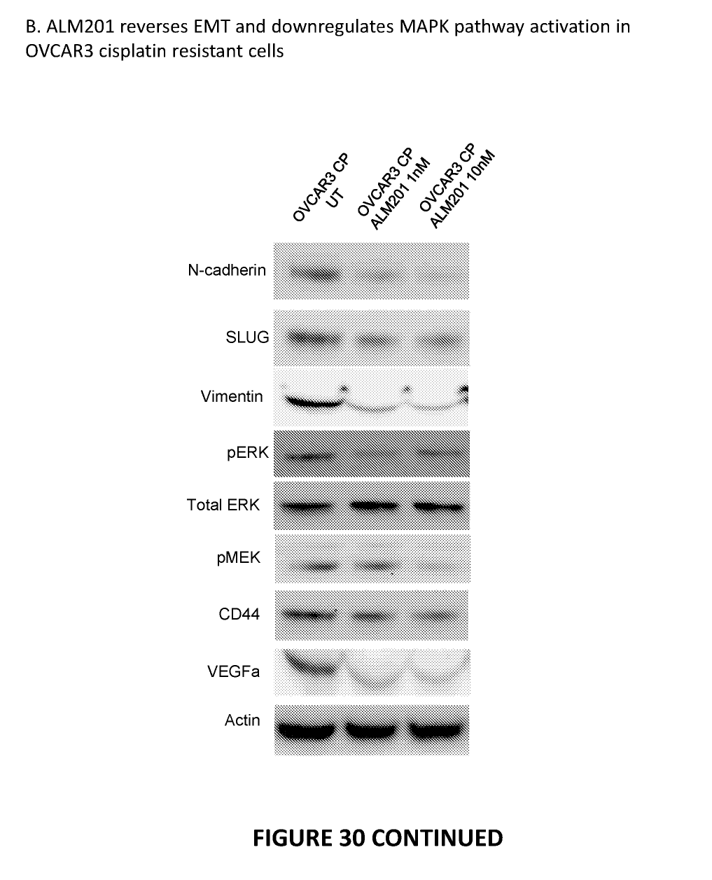

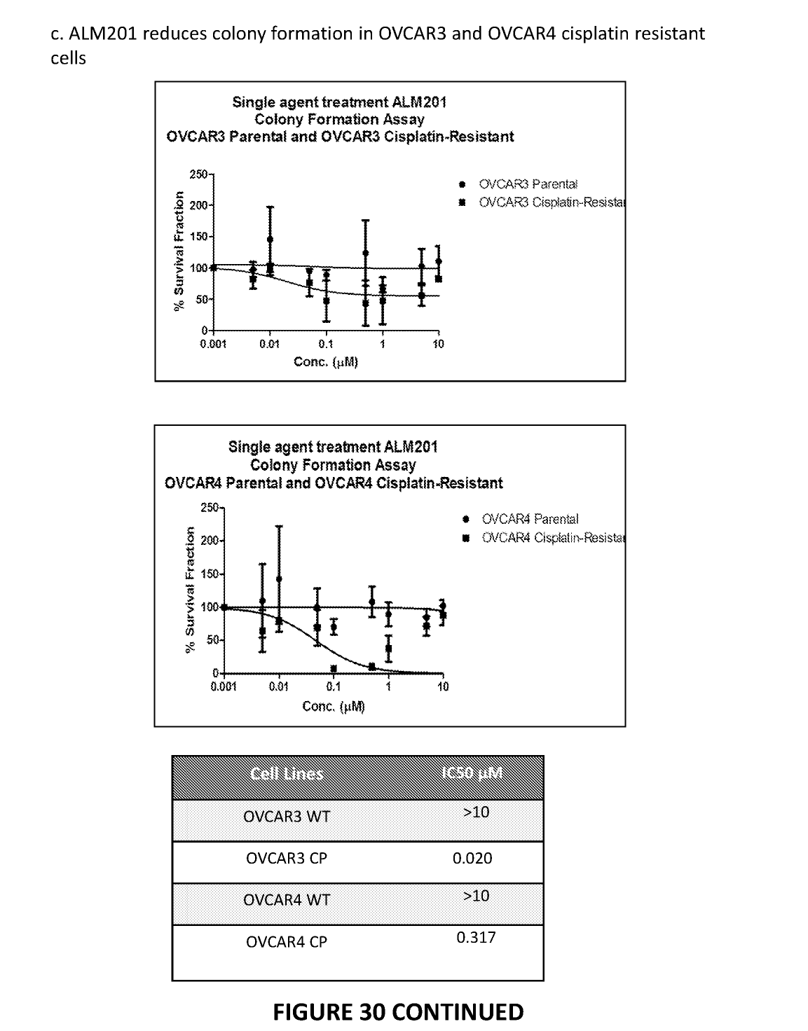

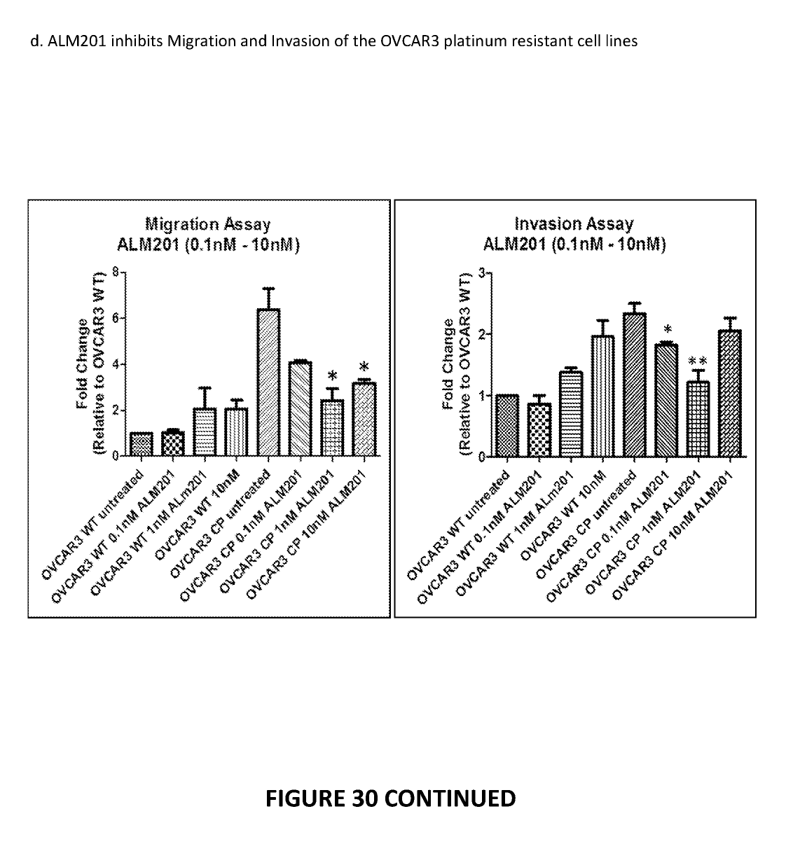

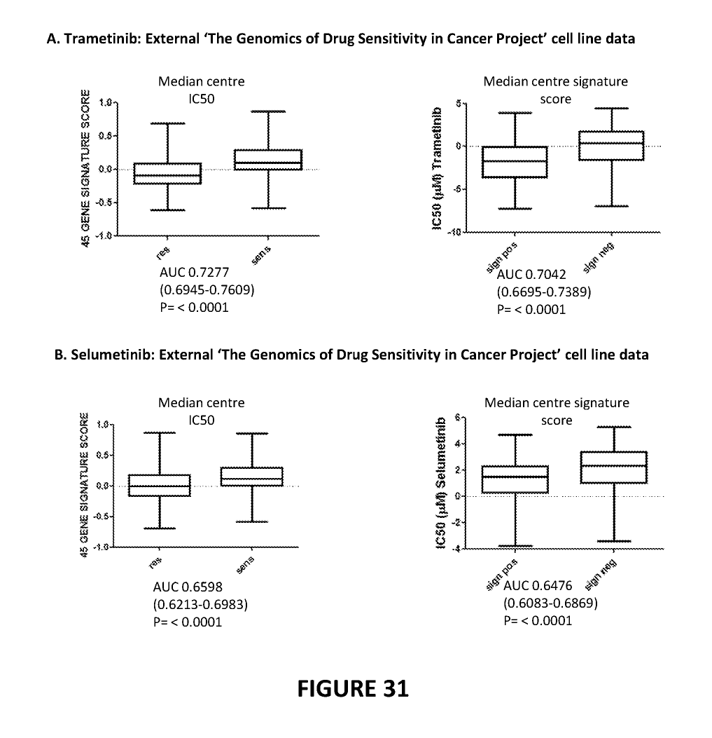

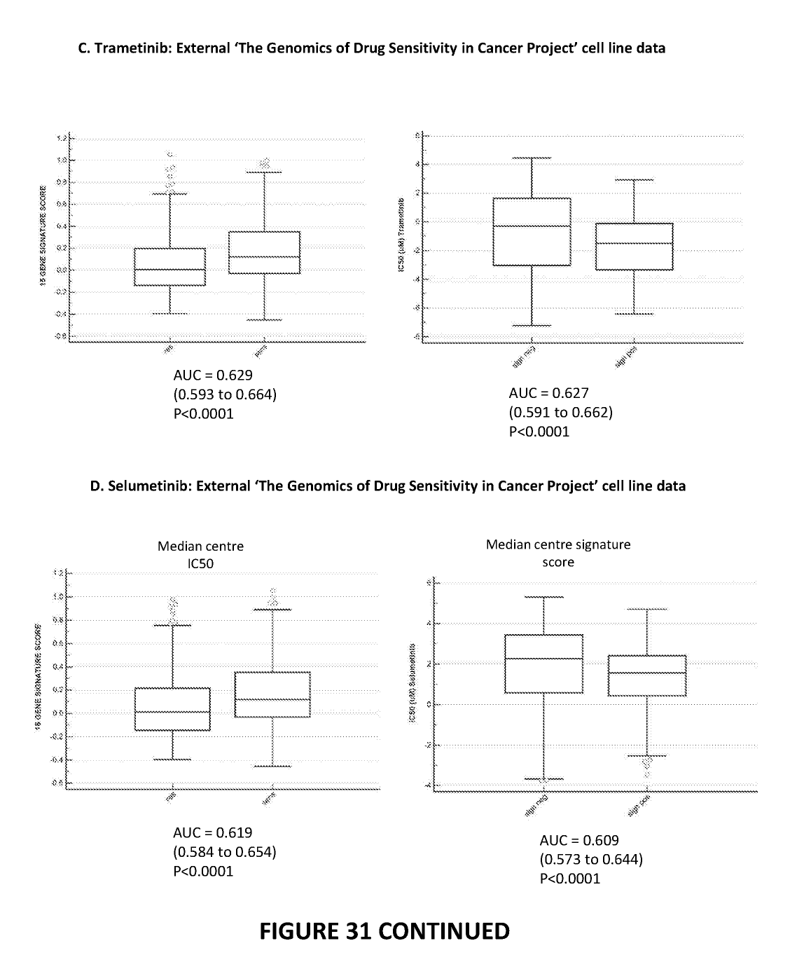

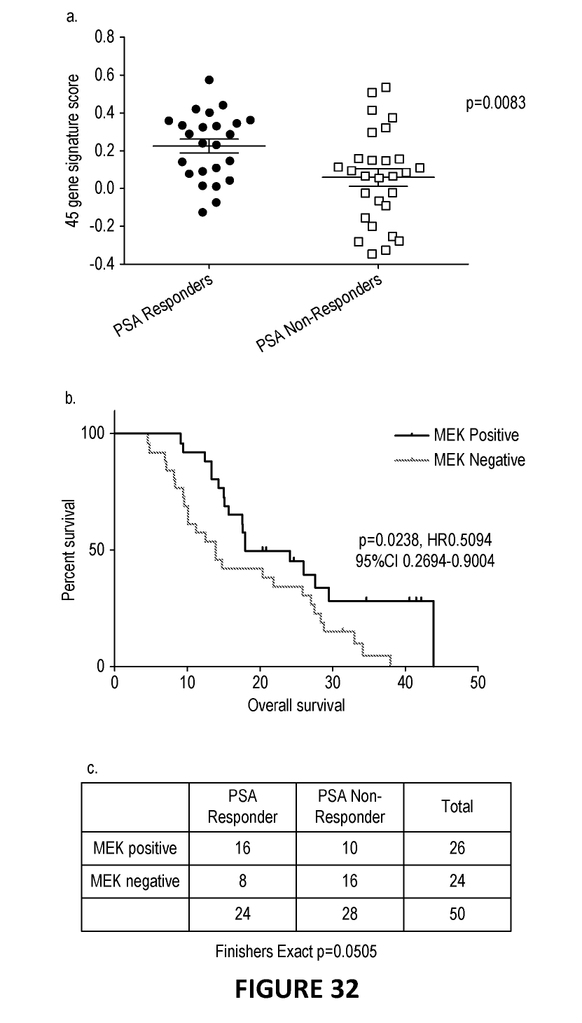

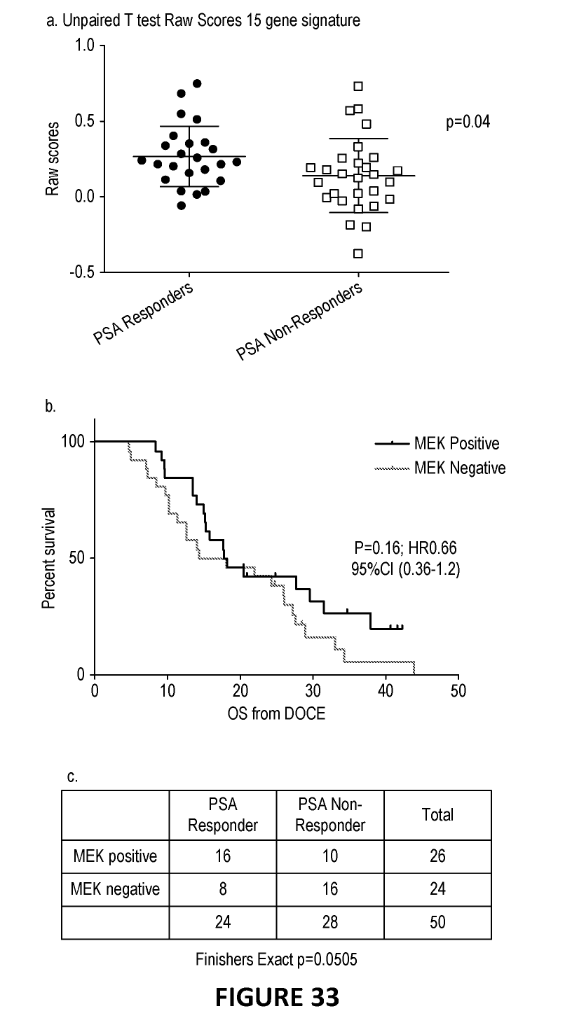

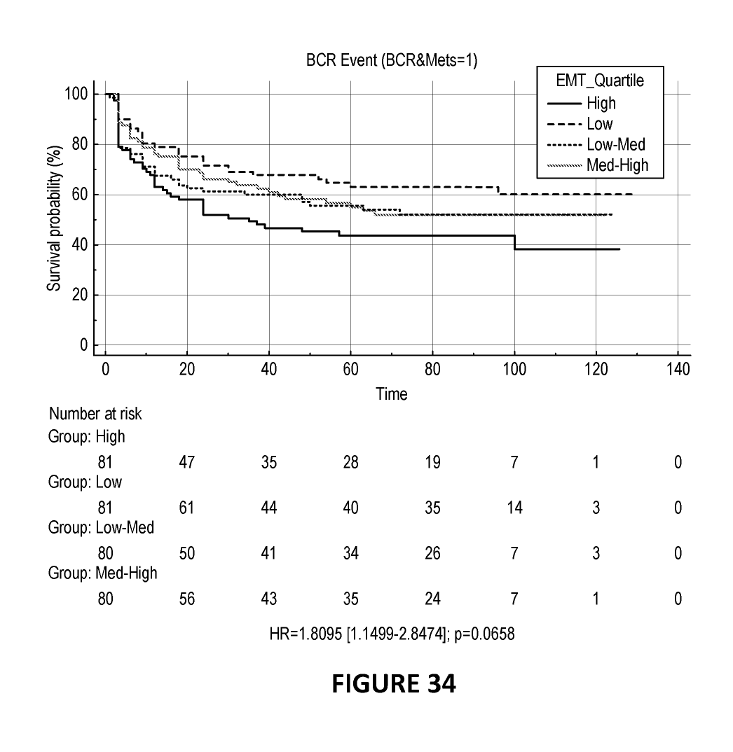

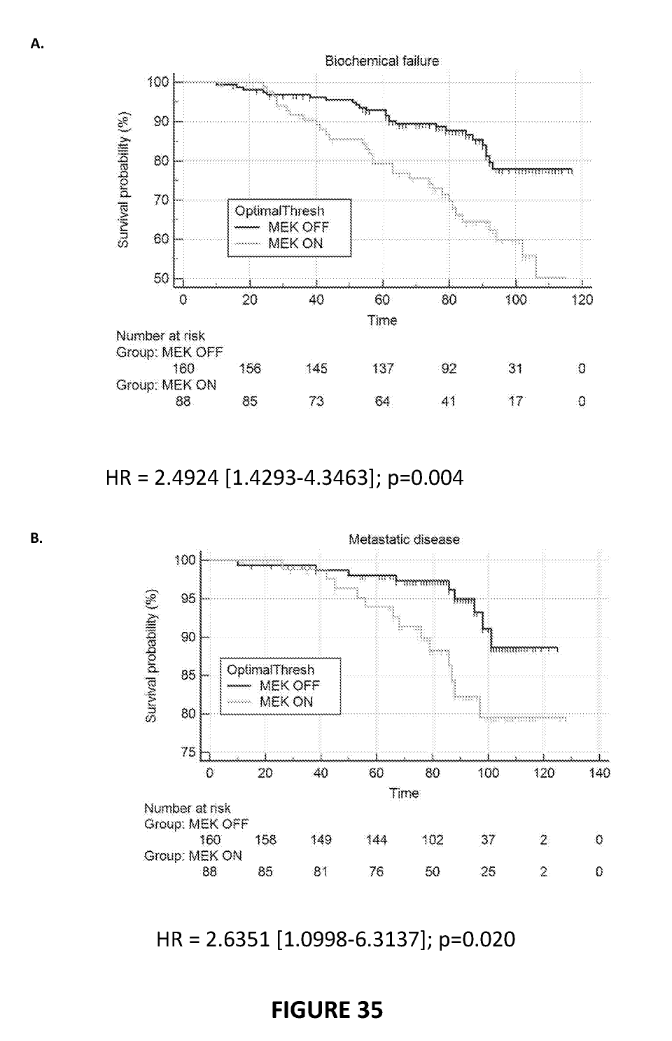

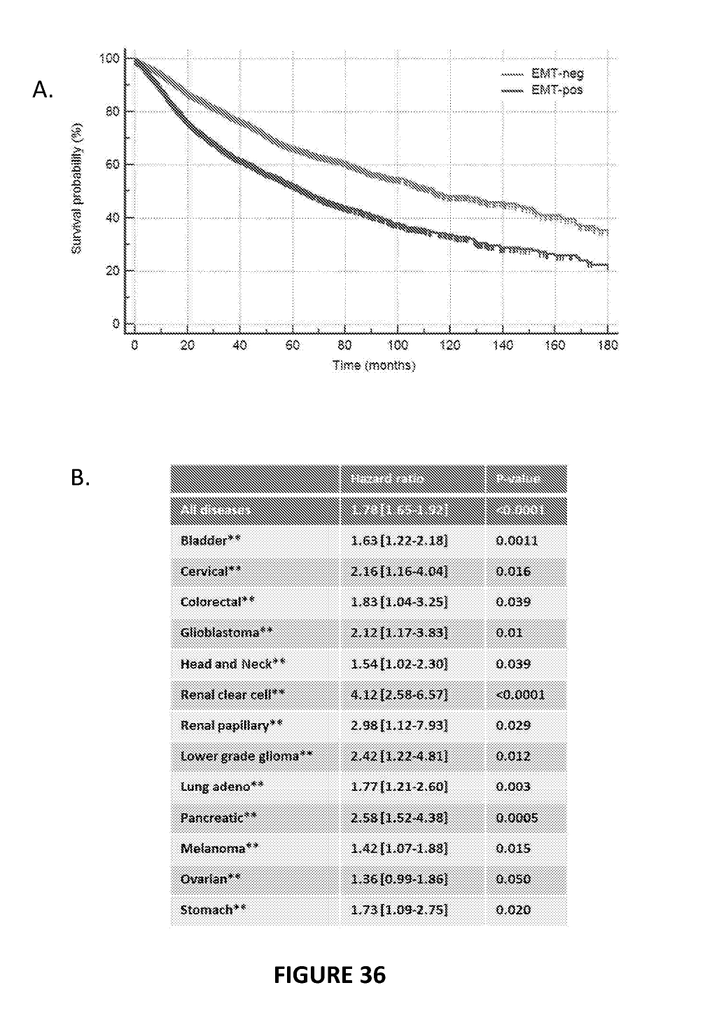

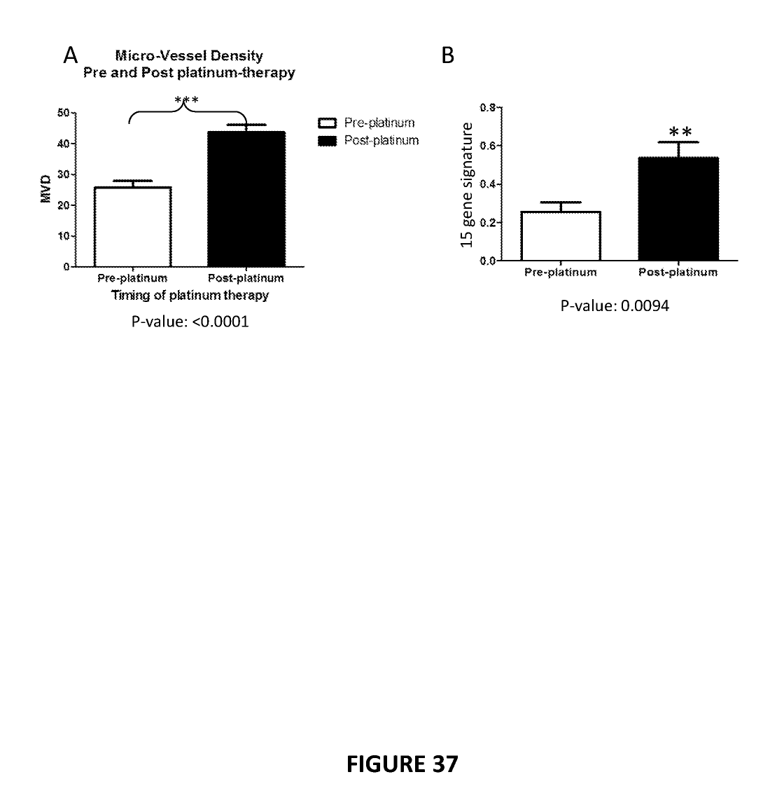

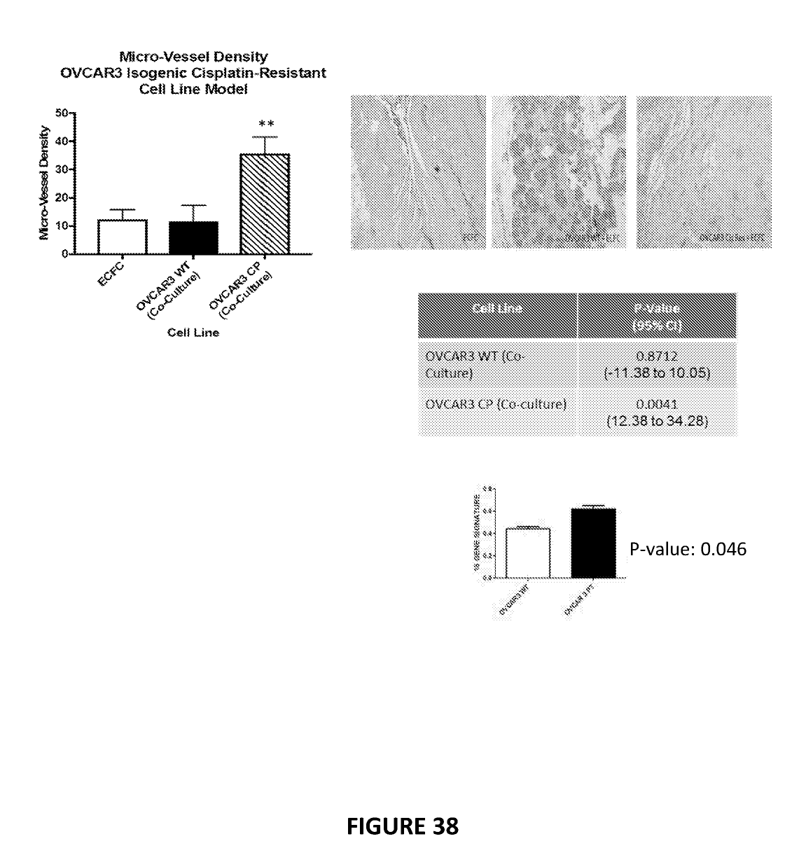

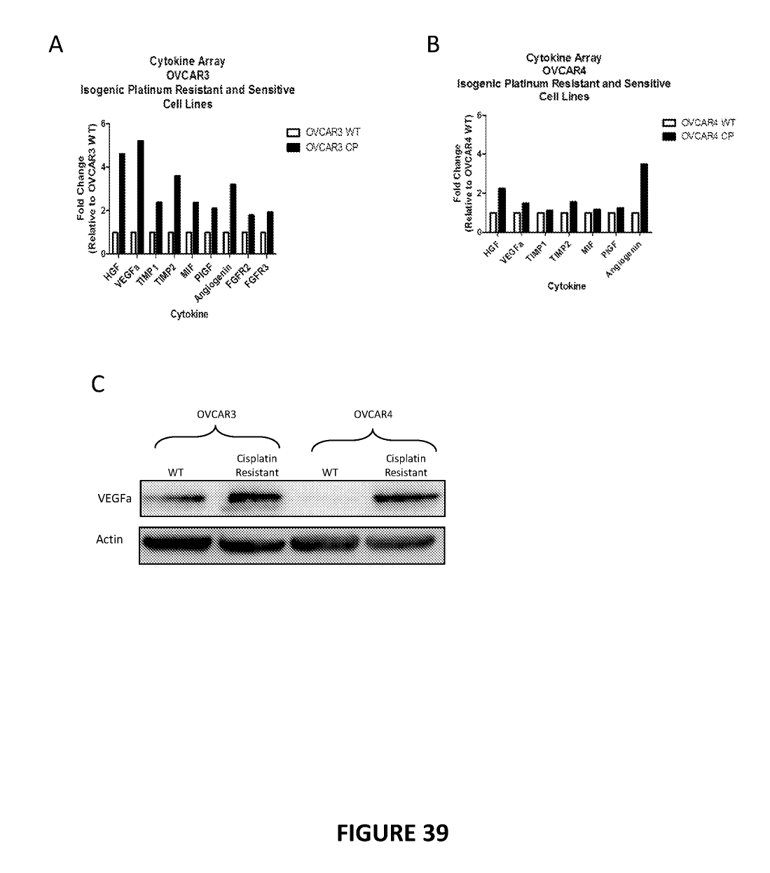

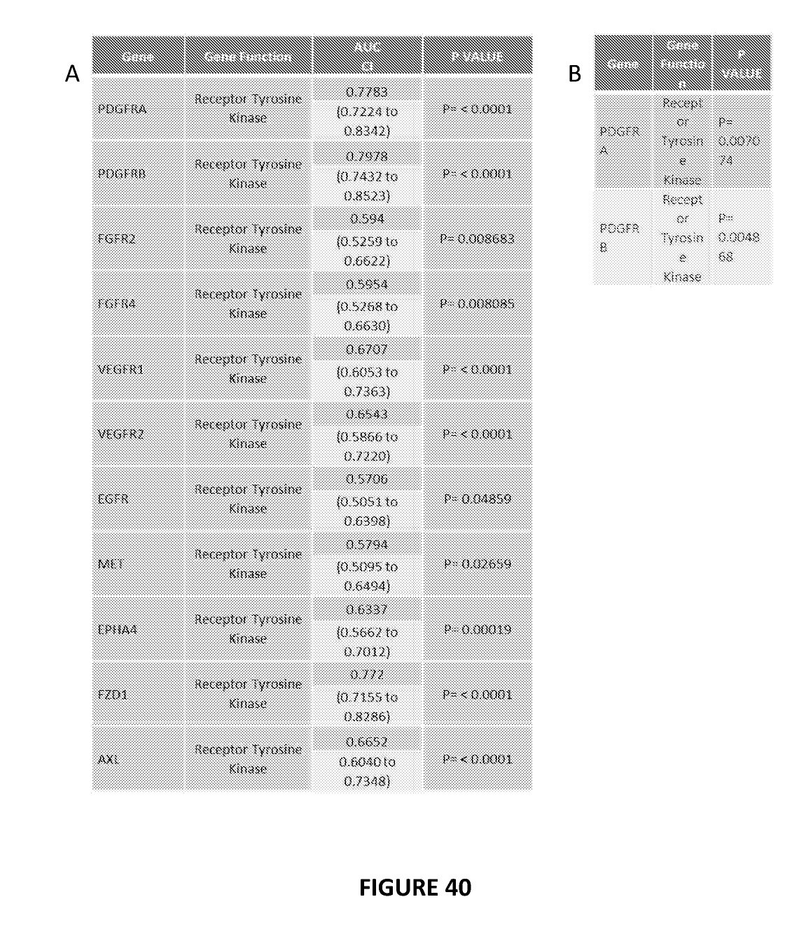

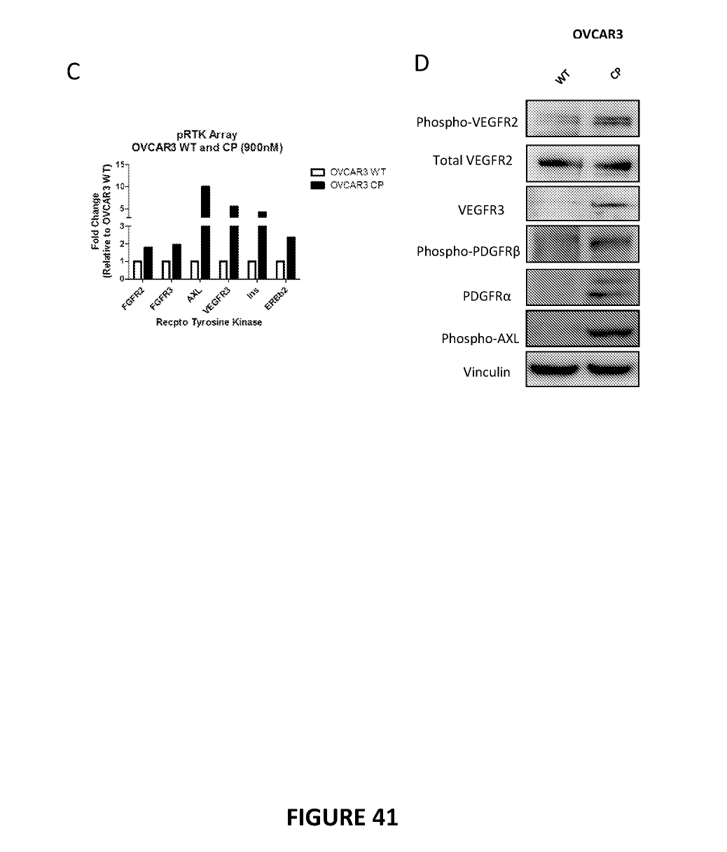

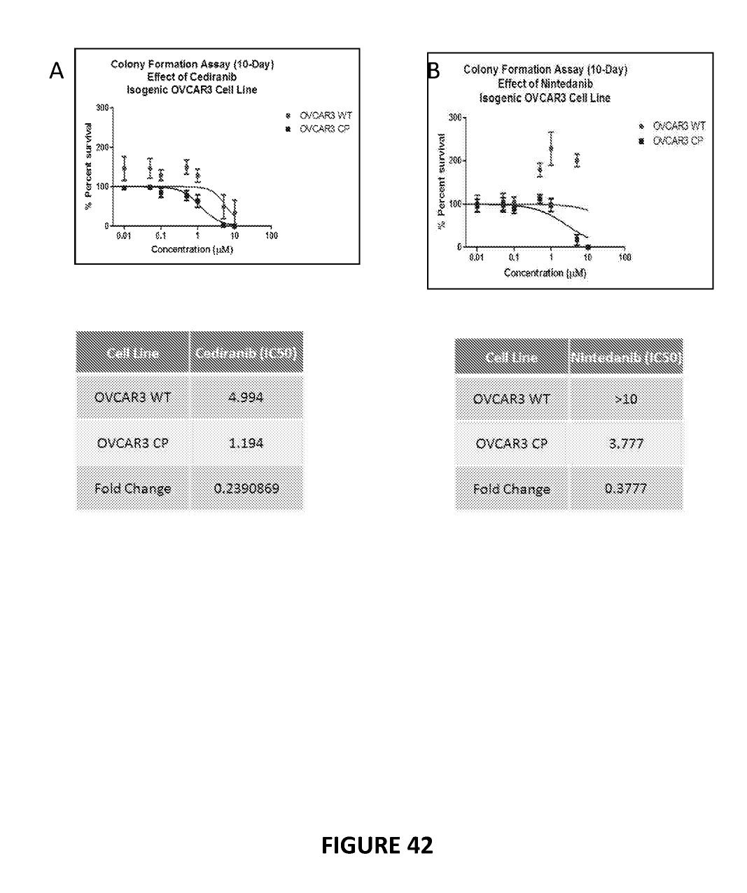

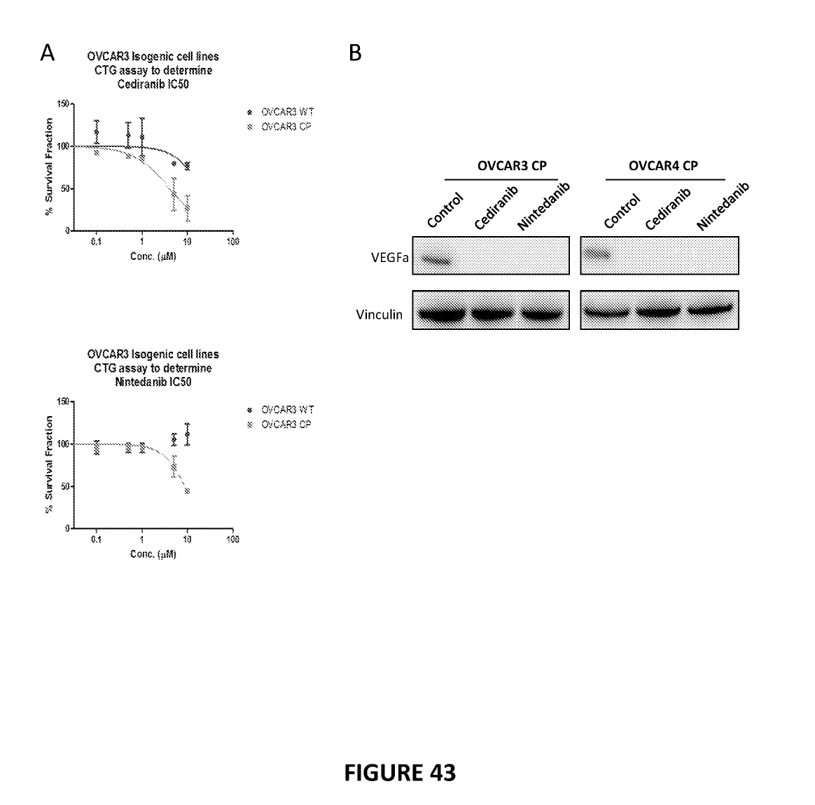

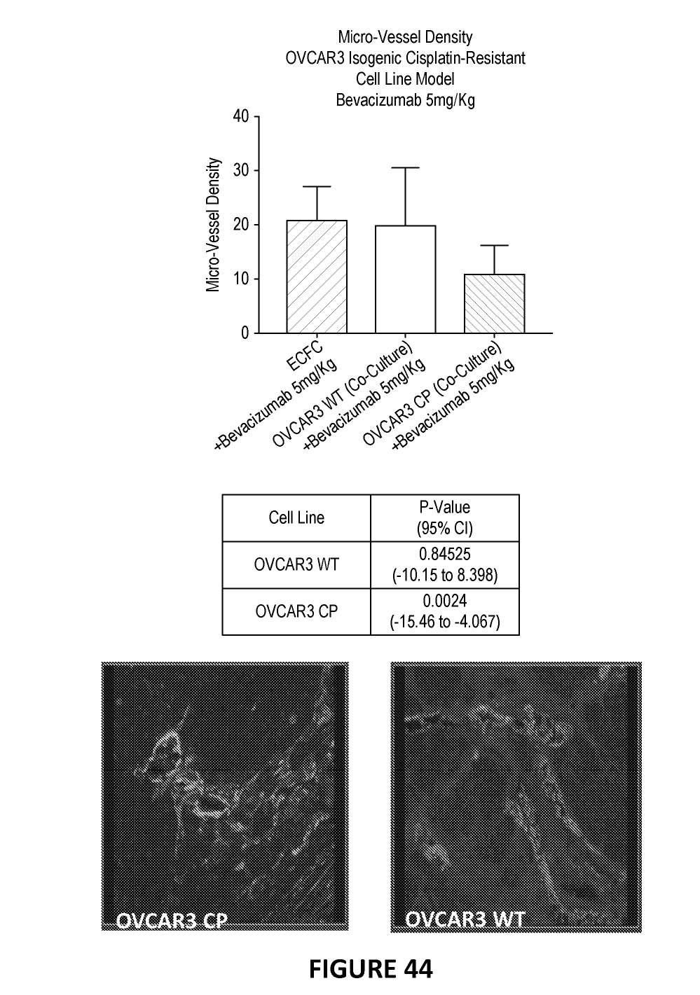

We have defined 3 molecular subgroups of High grade serous ovarian cancer (HGSOC), an Angiogenesis subgroup (HGS1), an Immune subgroup (HGS2) and an Angio_Immune subgroup (HGS3) (FIG. 1a) using gene expression data from 265 FFPE HGSOC samples obtained from treatment naive patients but who were treated with carboplatin+paclitaxel or carboplatin only Standard of Care (SoC) chemotherapy (Gourley, Kennedy et al., manuscript in preparation). Functional analysis of the gene clusters revealed one subgroup was characterised by up-regulation of immune response genes (Immune subgroup), a second by up-regulation of angiogenesis/vascular development genes (Angio subgroup) and a third by up-regulation of both immune response and angiogenesis/vascular development genes (AngioImmune subgroup). Functional analysis of the gene clusters revealed that two of the 4 gene clusters had no significantly enriched processes (PS clusters 1 & 3), the third was characterised by Immune processes (PS cluster 2) and the fourth by Angiogenesis processes (PS cluster 4) (FIG. 21). Patients within these 3 molecular subgroups respond differently to SoC surgery and chemotherapy. The Immune group (green) has the best prognosis, represented by both increased progression-free survival (PFS) (HR=0.60 (0.44-0.82) compared to Angio_Immune subgroup and HR=0.64 (0.49-0.92) compared to Angio subgroup) and overall survival (OS) (HR=0.58 (0.41-0.82) compared to Angio_Immune subgroup and (HR=0.55 (0.37-0.80) compared to Angio subgroup), while the Angio (blue) and Angio_Immune (red) subgroups respond similarly (FIGS. 1b and 1c). We have defined this as the Discovery dataset.

### The 45 Gene Signature is Prognostic in HGSOC

The Angio_Immune subgroup is defined by the 45-gene signature. We hypothesised that the Angio_Immune group would be prognostic in the context of SoC treatment in ovarian cancer. We therefore investigated this in the treatment naive Discovery dataset. The 45-gene signature was associated with worse prognosis (PFS HR 1.6403 (1.2252-2.1960) p=0.0002 and OS HR 1.6562 (1.2169-2.2540) p=0.0004) and therefore predicted response to cisplatin based therapy (FIG. 1c).

### Effect of Cisplatin Treatment on Molecular Subgroups

We wanted to investigate the effect of platinum treatment on the previously identified molecular subgroups. To do this, we analysed 48 matched (from the same patient) pre-chemotherapy and post-chemotherapy samples by gene expression analysis on the ovarian DSA™. Each of the samples were then scored with each of the 3 gene signatures, the 22 Angio signature, the 63 gene Immune signature and the 45 Angio_Immune signature. This analysis allowed us to define which of the 3 molecular subgroups, the samples fell into. This analysis demonstrated that treatment with cisplatin based chemotherapy caused samples to move between subgroups, specifically more of the post-chemotherapy samples were aligned with the Angio_Immune subgroup, rather than the immune and Angio subgroups. 40% of the pre-treatment patient samples were aligned with the Angio_Immune subgroup which increased to 54% post-chemotherapy (FIG. 1d). This was especially evident in the Immune group where 52% of samples previously associated with the good prognosis Immune group pre chemotherapy treatment, were found to be associated with the bad prognosis subgroup, the Angio-Immune group post-chemotherapy. This suggests that samples move into the Angio_Immune group and out of the Immune groups after cisplatin treatment. Hence the Angio_Immune subgroup represents a subgroup of tumours which are cisplatin insensitive upfront but which also provide a mechanism of acquired resistance post-chemotherapy treatment. 12 matched pre and post-platinum chemotherapy patients sample were stained with CD31 to assess micro-vessel density (MVD) and determine whether platinum therapy promotes an increased tumour vascular infiltration. The post-platinum chemotherapy patient samples have a higher 15-gene signature score relative to their paired treatment-naïve pair (paired t-test; p-value: 0.0094) (FIG. 37). Quantification of MVD in the paired samples demonstrates that the post-platinum patient treatment samples have a higher MVD relative to their paired treatment-naïve pair (paired t-test; p-value: 0.0001) (FIG. 37).

Cisplatin Resistant Cell Line Models have Elevated 45 and 15-Gene Signature Scores

We used a number of cisplatin-sensitive and -resistant cell lines to model the Angio_immune group in vitro including the A2780 and A2780CP and a further cisplatin-sensitive and -resistant cell line generated in-house using the HGSOC OVCAR3 cell line. As high-grade serous ovarian cancer accounts for 70% of ovarian cancers (Seidman et al. 2004) we used the OVCAR3 cell line as they have been confirmed as high-grade serous (Domcke et al. 2013). Although this cell line was generated from a patient following treatment with platinum based chemotherapy, our research has demonstrated that this cell line remains sensitive to cisplatin treatment. The cisplatin resistant OVCAR3 cells had an IC50 of 0.29 μM compared to the cisplatin sensitive cells which had an IC50 of 0.066 μM representing a 4.4-fold difference in IC50 doses (FIG. 2a).

Furthermore, both cisplatin resistant cells (A2780CP and OVCAR3PT) had significantly increased 45-gene signature scores (Angio_Immune) compared to their sensitive counterparts (p=0057 and p=0.0244 respectively) (FIGS. 2b and 2c). Additionally the OVCA3PT cells had elevated 15 gene signature score compared to OVCAR3 sensitive counterparts (p=0.045) (FIG. 11c). This was specifically related to the Angio_Immune subgroup as the 22-gene Angio signature scores remained unchanged, and the 63-gene Immune group signature was decreased between resistant and sensitive cells (FIGS. 2b and 2c). It has been previously shown that the MAPK pathway is a mechanism of cisplatin resistance in multiple solid tumours including ovarian cancer (Schwarz et al., 2015, Patch et al., 2015, Benedetti et al., 2008, Eleveld et al., 2015). We therefore hypothesised that MAPK signalling may have been driving the Angio_Immune subgroup. Interestingly, both cisplatin resistant A2780 and OVCAR3 (A2780-CP and OVCAR3-CP) cells had increased MAPK signalling compared to the cisplatin sensitive counterparts, as measured by phospho-MEK and phospho-ERK protein expression (FIG. 2d, top panels).

Since analysis of the clinical samples of the Discovery dataset demonstrated that the 45-gene signature could predict response to cisplatin based SoC treatment, we used a panel of 15 ovarian cell lines to investigate this further. These cells lines were analysed by DNA microarray analysis using the Ovarian Cancer DSA™ and signature scores were generated as previously described. The cell lines were also used to perform colony formation assays with Pharmaceutical grade cisplatin. A ROC curve was generated. Cisplatin resistance (res) or sensitivity (sens) was defined based on the median of the IC50 values and correlation with signature scores and AUC scores determined. This demonstrated that in cell line models the 45-gene signature could predict resistance to cisplatin upfront as shown by the increased 45-gene signature scores in resistant ovarian cell line panels (res) (AUC 0.7917 (0.6350-0.9483), p=0.0008) (FIG. 2e). Additionally ROC generated using the median of the signature scores demonstrated that signature positive (sign pos) were resistant to cisplatin compared to signature negative (sign neg) cells (AUC 0.6838, (0.5184-0.8492) P=0.03477). In contrast the Angio or the Immune signatures did not predict resistance to cisplatin.

### The Angio_Immune Subgroup is Driven by MAPK Signalling

To further investigate whether the Angio_Immune group was driven by this MAPK signalling pathway, we performed in silico analysis of the gene expression data from the Discovery dataset. To do this we firstly identified 3 different gene lists representing MAPK activation from the literature (Dry et al., 2010, Loboda et al., 2010, Creighton et al., 2006). We used these gene lists separately to perform semi-supervised hierarchical clustering of the Discovery dataset which were then compiled to generate a refined gene list representing a MAPK driven patient population (‘MEK ON’, represented by the red box) (FIG. 3a). In total, this analysis identified 101 samples of the 285 Discovery dataset as having increased MAPK signalling. The overlap of these MAPK driven patients with the 3 identified molecular subgroups is represented by the Venn diagrams in FIG. 3b. In total, 77% ( 56/73 patients) of the ‘MEK ON’ patients fall within the Angio_Immune group, whilst only 6% ( 4/63 patients) and 17% (10/60 patients) of the ‘MEK ON’ patients fall into the Angio and Immune subgroups respectively (FIG. 3b). This suggests that the Angio_Immune subgroup represents the ‘MEK ON’ population and this molecular subgroup is driven by MAPK signalling.

Reverse Phase Proteomic Array (RPPA) data was utilised from The Cancer Genome Atlas (TCGA) dataset. The continuous Phospho-MAPK (pMAPK) scores (serine 217/221) and total MAPK scores were down loaded from TCGA (http://bioinformatics.mdanderson.org/main/TCPA). Phospho-MAPK scores were calculated as a ratio of total MAPK. Gene signature scores were then correlated with the RPPA data and the Angio_Immune gene signature was specifically found to correlate with pMAPK serine 217/221 expression using ROC analysis (FIG. 3c). Both the Angio and Immune gene signatures did not significantly correlate with pMEK expression. Significant differences in pMAPK expression were observed between the Angio_Immune subgroup and the Angio and Immune subgroups respectively (p=0.0057 and p=0.0250) (FIG. 3c). This indicates that the Angio_Immune subgroup is being driven by the MAPK pathway and is associated with MEK expression.

Furthermore the 45-gene signature could predict sensitivity to the MEK inhibitor Trametinib (Mekinist, GSK) as demonstrated by the increased 45-gene signature score in sensitive ovarian cell line panels (sens) (AUC 0.72 (0.5778-0.8690) p=0.009066) (FIG. 3d). Additionally ROC generated using the median of the signature scores demonstrated that signature positive (sign pos) were sensitive to Trametinib compared to signature negative (sign neg) cells (AUC 0.7147 (0.5674-0.8620) P=0.01170). This phenotype was specifically related to the 45-gene signature as the Angio and Immune signatures did not predict sensitivity. The Angio_Immune subgroup may now be defined by the 45-gene signature or ‘MEK signature’.

Assessment of 45 Gene Expression Signature with Drug Response in Sanger Cell Lines

Gene expression data (Affymetrix U219 chip) downloaded from https://www.ebi.ac.uk/arrayexpress/experiments/E-MTAB-3610/ and cell lines sensitivity data with regards to the MEK inhibitors Trametinib and Selumetinib downloaded from http://www.cancerrxgene.org/. The cancer cell lines were scored with the 45 gene AngioImmune signature and correlations of signature score and IC50 response were determined as before: based on median centred IC50 doses against signature scores and vice versa plotting median centered signature scores against IC50 doses. ROC analysis demonstrated the 45 gene signature could predict response to both MEK inhibitors in these cell lines. For Trametinib there was 881 solid tumour cell lines which had both gene expression data and drug IC50 data and for Selumetinib there was 760 solid tumour cell lines which had both gene expression data and drug IC50 data.

### The 45-Gene Signature is Altered by KRAS Status and MEK Inhibitors

As mentioned previously, it has been found that 11% of HGSOC have KRAS amplification and 12% have BRAF amplification, highlighting the potential that drugs targeting the RAS/RAF/MEK/ERK pathway may have utility in HGSOC. The link between KRAS status and MEK was assessed using three publically available datasets; E-GEOD-55624 which profiles KRAS mutant cancer cells treated with a MEK inhibitor, E-MEXP-3557 which transcriptionally profiles human KRAS mutant and wildtype cells and E-GEOD-12764 which transcriptionally profiled MCF10 cells with overexpressed HRAS or MEK1. Using the E-GEOD-55624 dataset, following treatment of SW480 cells which harbour a KRAS mutation (G12D), with a MEK inhibitor exhibited a reduction in the 45-gene Angio_Immune signature scores at both 4 and 16 hours (p=0.0055 and p=0.0143), in comparison to DMSO control (FIG. 4a). Of note, the reduction is enhanced post MEK inhibitor treatment at 16 hours. This effect was specific to the Angio_Immune subgroup, as both the Angio and Immune signatures remained unchanged. Furthermore, data from the E-MEXP-3557 dataset showed that KRAS mutant (KRAS MT) HCT116 cells had an association with the Angio_Immune subgroup with higher 45-gene signature scores compared to the wildtype HKH2 cells (KRAS WT) (p=0.0072) (FIG. 4b). No association was observed between KRAS status and either the Angio or Immune gene signatures. Moreover, MCF10 epithelial breast cells transfected with either HRAS or MEK1 also show an association with the 45-gene signature (p=0.0004 and p=<0.0001 respectively) compared to the empty vector (EV) (FIG. 4c). As expected, no significant changes or association were observed between HRAS or MEK1 and the Angio or Immune subgroups. Moreover, inhibition of MEK with Trametinib significantly decreases the 45-gene Angio_Immune signature score in OVCAR3 cells (p=0.0011) (FIG. 4d). This data suggests that the MEK signature is altered by KRAS status and MEK inhibitors.

a) MEK inhibition in cisplatin resistant OVCAR3 cells, re-sensitises cells to cisplatin

b) To further investigate the role of MEK signalling in driving resistance to cisplatin, OVCAR3-WT and OVCAR3-CP cells were treated with either cisplatin alone or a combination of cisplatin and the MEK inhibitor Trametinib. Following treatment of OVCAR3-WT cells with increasing concentrations of cisplatin, OVCAR3-CP cells formed more colonies than OVCAR3-WT cells. However, the addition of 0.5 uM Trametinib to increasing concentrations of cisplatin resulted in a decrease in colony formation of both OVCAR3-WT and OVCAR3-CP cells (FIG. 5a, red line). This resulted in a 2 fold decrease in the IC50 of OVCAR3-WT cells from 0.066 uM to 0.033 uM following the addition of Trametinib to cisplatin. The addition of Trametinib to increasing concentrations of cisplatin resulted in a 4.8 fold decrease in the IC50 of OVCAR3-CP cells from 0.029 uM to 0.06 uM. Importantly, there was a greater fold change in the IC50 cells following the addition of Trametinib to cisplatin for OVCAR3-CP cells (FIG. 5b, orange line).

### The Angio_Immune Subgroup has Increased EMT Signalling

MAPK is known to phosphorylate SLUG and other key players of the SNAIL/SLUG transcription factors, to induce epithelial-mesenchymal transition (EMT) which is known to be a contributing mechanism to angiogenesis and of progressive disease (Virtakoivu et al., 2015). Gene expression profiling showed that a range of EMT associated genes had higher expression levels in the Angio-Immune subgroup as opposed to the Angio and Immune subgroups. This includes significantly increased expression of VIMENTIN, AXL, TWIST1, SNAIL and SLUG in the Angio_Immune subgroup (p=<0.001) compared to the Angio and Immune subgroups (FIG. 6a). Likewise, in the E-GEOD-58582 dataset, MCF7 breast cells transfected with SNAIL show a positive association with the 45-gene signature (p=0004) (FIG. 6b). In sum, the Angio_Immune subgroup has increased EMT signalling. As a result, the Angio_Immune subgroup may now be defined forthwith as the ‘EMT signature’.

### Activation of the EMT Phenotype is Enhanced in Cisplatin Resistant Ovarian Cell Lines

Above we showed that MAPK signalling may be driving the Angio_Immune subgroup. Interestingly, cisplatin resistant OVCAR3 and OVCAR 4 cells had increased MAPK signalling compared to the cisplatin sensitive counterparts, as measured by phospho-MEK and phospho-ERK protein expression (FIG. 7a and FIG. 2d). Moreover, we already demonstrated that the 45- and -15-gene signature not only predicts cisplatin resistance but is also elevated by EMT. Therefore we decided to investigate the relationship between cisplatin resistance in-vitro and an associated EMT phenotype utilising both cisplatin-sensitive and -resistant cell lines generated in-house; HGSOC OVCAR3 and OVCAR4 cell lines. It appears that cisplatin resistant ovarian cells (OVCAR3 CP and OVCAR4 CP) exhibit activation of EMT signalling as demonstrated by the upregulated protein expression of Vimentin, N-cadherin and SLUG in conjunction with decreased levels of E-cadherin, compared to their wildtype counterparts (FIG. 7b). These observed changes in EMT markers are all indicative of EMT activation. In addition, cisplatin resistant OVCAR3 and OVCAR4 cells also exhibit significantly increased mRNA expression levels of some EMT markers, including N-cadherin, SLUG, SNAIL, Vimentin, TWIST and TGF-β3. This is shown by the greater fold change difference in OVCAR3 CP and OVCAR4 CP relative to the wildtype cells (FIGS. 7c and d). Moreover, cisplatin resistance in OVCAR3 and OVCAR4 shows greater cell migration and an enhanced migratory phenotype (FIG. 7e), hence suggesting the activation of EMT and Angiogenic phenotypes.

### ALM201 Reverses the EMT Phenotype in OVCAR3 and 4 Cisplatin Resistant Cells

We used the EMT on cell line Kuramochi and the OVCAR3 and 4 cisplatin resistant cells to examine the effects of ALM201 on EMT markers and associated phenotypes. ALM201 treatment caused reduced MAPK signalling and EMT signalling in the Kuramochi cell line (FIG. 30a). The same effect was seen in the OVCAR3 cisplatin resistant cell line (FIG. 30b). Additionally in the OVCAR3 and OVCAR4 cells treatment with ALM201 inhibited the proliferation capacity (FIG. 30c), and in the OVCAR3 cisplatin resistant cells, ALM201 inhibited the migration and invasion potential of the cells (FIG. 30d).

### The EMT Signature Predicts Resistance to Inhibitors of the SRC Pathway

Further investigation suggests that both the MAPK and SRC signalling pathways signal as parallel pathways and may contribute to drug resistance. Treatment with 1 μM Saracatinib (SRCi) over 24 hours, reduces the protein expression of pSRC (left, top panel) whilst increasing the protein levels of pERK (left, third panel). In contrast to this, treatment with 1 μM Trametinib (MEKi) over 24 hours exhibits the opposite effect, enhancing pSRC protein levels (right, third panel) and decreasing pERK protein expression (right, top panel) (FIG. 8a). Resistance (res) or sensitivity (sens) to SRC inhibitors was defined based on the median of the IC50 values and correlation with 45-gene signature scores and AUC scores determined. This demonstrated that in cell line models the EMT signature could predict resistance to SRC inhibitor, Saracatinib (AUC 0.8867 (0.7757-0.9978), p=0.00019) as shown by the increased 45-gene signature score in resistant cell lines (res) (FIG. 8b). This suggests that the SRC and MAPK pathways act in parallel whereby the inhibition of one signalling cascade leads to the activation of the other. To conclude, the EMT signature predicts response to MEK inhibitors (sensitivity) and SRC inhibitors (resistance). Theoretically, targeting both pathway would be a plausible therapeutic combination.

### The EMT Signature Detects a Poor Prognosis Subgroup in Colon Cancer (CRC) and Non-Small Cell Lung Cancer (NSCLC)

As the Angio_Immune group is driven by MAPK signalling, we hypothesised that the EMT signature may also have prognostic utility in alternative disease indications, namely colon cancer (CRC) and non-small cell lung cancer (NSCLC) which have high incidence of alterations in the MAPK pathway. We therefore investigated this in two publically available colon datasets both in the context of treatment (Marisa GSE40967 and Jorissen GSE14333) and one NSCLC dataset which incorporates an untreated population (Okayama GSE31210). The Marisa dataset consisting of 566 Stage I-IV colon cancers, had the MEK defined subgroup present in sample cluster 3 (C3) following hierarchical clustering (FIG. 9a, red box). The MEK subgroup (C3) was associated with worse prognosis (RFS, p=0.037) (FIG. 9b, red line). Additionally, the 45-gene EMT signature described as ‘MEK ON’ was associated with poor prognosis, (RFS HR 1.594 (1.095-2.323), p=0.0063) (FIG. 9c, red line). The Jorissen dataset consisting of 260 Stage I-IV colon cancers also showed a poor prognostic subgroup detected by the 45-gene EMT signature, (DFS HR 2.454 (1.205-4.999), p=0.0014) (FIG. 9d, red line).

In relation to NSCLC, the Okayama dataset consisting of Stage I and II untreated NSCLC samples also had the MEK defined subgroup present in sample cluster 4 (C4) following hierarchical clustering (FIG. 10a, red box). The MEK subgroup (C4) was associated with worse prognosis (p=0.0004) (FIG. 10b, red line). Additionally, the 45-gene EMT signature described as ‘SIGN POS’ was associated with poor prognosis, (PFS HR 3.045 (1.631-5.686), p=0.0005) (FIG. 10c, red line). The Okayama dataset confirmed a poor prognostic subgroup detected by the 45-gene EMT signature, (OS HR 2.872 (1.271-6.489), p=0.0312) (FIG. 10d, red line). In sum, the 45-gene EMT signature detects a poor prognosis subgroup of patients driven by the MAPK pathway activation and is therefore independent of treatment.

### The 15-Gene Signature Predicts Response to Cisplatin and is Elevated in Cisplatin Resistant Cells

The Angio_Immune subgroup may now be defined forthwith as the 15-gene signature. AS with the 45-gene signature, we hypothesised that the Angio_Immune group would be prognostic in the context of SoC treatment in ovarian cancer. We therefore investigated this in the treatment naive Discovery dataset. The 15-gene signature was also associated with worse prognosis (PFS, HR=1.3564 [1.0156-1.8117]; p=0.0279 and OS, HR=1.3464 [0.9901-1.8308]; p=0.0441) and could predict response to cisplatin based therapy (FIG. 11a).

Since analysis of the clinical samples of the Discovery dataset demonstrated that the 15-gene signature could similarly predict response to cisplatin based standard of care treatment, we used a panel of 15 ovarian cell lines to investigate this further. These cells lines were analysed by DNA microarray analysis using the Ovarian Cancer DSA™ and signature scores were generated as previously described. The cell lines were also used to perform colony formation assays with Pharmaceutical grade cisplatin. A ROC curve was generated. Cisplatin resistance (res) or sensitivity (sens) was defined based on the median of the IC50 values and correlation with signature scores and AUC scores determined. This demonstrated that in cell line models the 15-gene signature could predict resistance to cisplatin upfront as shown by the increased 15-gene signature scores in resistant ovarian cell line panels (res) (AUC 0.6905 (0.5254-0.8556), p=0.2900) (FIG. 11b). Additionally ROC generated using the median of the signature scores demonstrated that signature positive (sign pos) were resistant to cisplatin compared to signature negative (sign neg) cells (AUC 0.0.6897 (0.5326-0.8468) P=0.02932). Furthermore, both OVCAR3 cisplatin resistant cells (OVCAR3PT) had significantly increased 15-gene signature scores (Angio_Immune) compared to their sensitive counterparts (0.046) (FIG. 11c).

The 15-Gene Signature is Also Associated with the MAPK Pathway

The potential link between the MAPK pathway and the 15-gene signature was assessed using two previously mentioned publically available datasets; E-MEXP-3557 and E-GEOD-12764. Data from the E-MEXP-3557 dataset showed that KRAS mutant (KRAS MT) HCT116 cells had an association with the Angio_Immune subgroup with higher 15-gene signature scores compared to the wildtype HKH2 cells (KRAS WT) (p=0.0443) (FIG. 12a). Moreover, MCF10 epithelial breast cells transfected with either HRAS or MEK1 also show an association with the 15-gene signature (p<0.0001) compared to the empty vector (EV) (FIG. 12b). Moreover, inhibition of MEK with Trametinib significantly decreases the 15-gene Angio_Immune signature score in OVCAR3 cells (p=0.0023) (FIG. 12c). This data suggests that the 15-gene signature is elevated by KRAS status or MEK1 overexpression and decreased by MEK inhibitors.

Utilising the Reverse Phase Proteomic Array (RPPA) data from The Cancer Genome Atlas (TCGA) dataset, phospho-MAPK scores were calculated as a ratio of total MAPK. Gene signature scores were then correlated with the RPPA data and AS previously mentioned Angio_Immune subgroup was specifically found to correlate with pMAPK serine 217/221 expression using ROC analysis (FIG. 3c). This confirms that the Angio_Immune subgroup is being driven by the MAPK pathway and the 15-gene signature is associated with MEK expression.

Furthermore the 15-gene signature could predict sensitivity to the MEK inhibitor Trametinib (Mekinist, GSK) as demonstrated by the increased 15-gene signature scores in ovarian cell line panels (sens) (AUC 0.7234 (0.5778-0.8690) p=0.0090) (FIG. 12d). Additionally ROC generated using the median of the signature scores demonstrated that signature positive (sign pos) cells were sensitive to Trametinib compared to signature negative (sign neg) cells (AUC 0.0.6897 (0.5326-0.8468) P=0.02932). The Angio_Immune subgroup may now be defined forthwith as the 15-gene signature or ‘MEK signature’.

### The 15-Gene Signature Exhibits Elevated EMT Signalling

As mentioned previously, MAPK is known to phosphorylate SLUG and other key players of the SNAIL/SLUG transcription factors, to induce epithelial-mesenchymal transition (EMT) which is known to be a contributing mechanism of progressive disease. Using the E-GEOD-58582 dataset, MCF7 breast cells overexpressing SNAIL show a positive association with the 15-gene signature (p=0.0015) (FIG. 13). In sum, the 15-gene signature is also associated with enhanced EMT signalling. As a result, the 15-gene signature may now be defined forthwith as the ‘EMT signature’.

### The 15-Gene EMT Signature Also Predicts Resistance to Inhibitors of the SRC Pathway

Further investigation suggests that both the MAPK and SRC signalling pathways signal as parallel pathways and may contribute to drug resistance. Resistance (res) or sensitivity (sens) to SRC inhibitor, Saracatinib was defined based on the median of the IC50 values and correlation with 15-gene signature scores (Sign Pos vs Sign Neg) and AUC scores determined (AUC 0.7289, (0.5544-0.9035), p=0.01454) (FIG. 14a). This demonstrated that in cell line models the 15-gene EMT signature could predict resistance to SRC inhibitor, Saracatinib (AUC 0.7698 (0.6054-0.9343), p=0.004), as shown by the increased 15-gene signature score in resistant cell lines (res) (FIG. 14b). This suggests that the SRC and MAPK pathways act in parallel whereby the inhibition of one signalling cascade leads to the activation of the other.

### The 15-Gene EMT Signature Detects a Poor Prognosis Subgroup in Colon Cancer (CRC) and Non-Small Cell Lung Cancer (NSCLC)

As the Angio_Immune group is driven by MAPK signalling, we hypothesised that the 15-gene

EMT signature may also exhibit prognostic utility in alternative disease indications; colon cancer and non-small cell lung cancer (NSCLC). We therefore investigated this in the two publically available colon datasets in relation to treatment (Marisa GSE40967 and Jorissen GSE14333) and one untreated NSCLC dataset (Okayama GSE31210). The Jorissen dataset consisting of 260 Stage I-IV colon cancers showed a poor prognostic subgroup detected by the 15-gene EMT signature, (DFS, p=0.0328) (FIG. 15a, red line). The Marisa dataset confirmed that the 15-gene signature was associated with worse prognosis (RFS, p=0.0161) (FIG. 15b, red line).

With regards to NSCLC, the Okayama dataset consisting of Stage I and II untreated NSCLC samples also associated with 15-gene signature with worse prognosis. The 15-gene EMT signature described as ‘SIG POS’ was associated with poor prognosis, (PFS, p=0.0024) (FIG. 16a, red line). The Okayama dataset confirmed a poor prognostic subgroup detected by the 15-gene EMT signature with reduced overall survival in the ‘SIG POS’ cohort, (OS, p=0.0396) (FIG. 16b, red line). Additionally the 15 gene signature is prognostic across a number of tissues p<0.0001 (FIGS. 36a and b). In sum, the 15-gene EMT signature detects a poor prognosis subgroup of patients across multiple diseases and is driven by the MAPK pathway activation and EMT signalling.

### The 45 Gene and 15 Gene Signatures are Predictive of Response to MEK Inhibitors

Furthermore the both the 45 and 15-gene signature could predict sensitivity to the MEK inhibitors, Trametinib (Mekinist, GSK) and Selumetinib from ‘The Genomics of Drug Sensitivity in Cancer Project’ cell line data. This is demonstrated by the increased 45-gene signature scores in a panel of cell lines treated with either Trametinib or Selumetinib (sens) (AUC 0.7277 (0.6945-0.7609) p<0.0001 and AUC 0.6598 [0.6213-0.6983]; p<0.0001 respectively) (FIGS. 31a and b). Additionally ROC generated using the median of the 45-gene signature scores demonstrated that signature positive (sign pos) cells were sensitive to Trametinib and Selumetinib compared to signature negative (sign neg) cells (AUC 0.7042 [0.6695-0.7389]; p<0.0001 and AUC 0.6476 [0.6083-0.6869]; p<0.0001 respectively).

Additionally, this is also demonstrated by the increased 15-gene signature scores in a panel of cell lines treated with either Trametinib or Selumetinib (sens) (AUC 0.629 (0.593-0.664) p<0.0001 and AUC 0.619 [0.584-0.654]; p<0.0001 respectively) (FIGS. 31c and 17d). Additionally ROC generated using the median of the 15-gene signature scores demonstrated that signature positive (sign pos) cells were sensitive to Trametinib and Selumetinib compared to signature negative (sign neg) cells (AUC 0.627 [0.591-0.662]; p<0.0001 and AUC 0.609 [0.573-0.644]; p<0.0001 respectively).

### Platinum Resistance Creates a Selection Pressure for an Angiogenesis Enriched Microenvironment

Microvessel density (MVD) is a measure of the angiogenic capacity of a tumor and as the angiogenic potential increases so too does its MVD. An in-vivo matrigel-plug assay was used to determine the micro-vessel density (MVD) in the OVCAR3 isogenic cell lines in co-culture with Human endothelial colony forming cells (EFCF). The OVCAR3 platinum-resistant cell lines have a higher MVD than the OVCAR3 platinum-naïve pair (p-value: 0.0041) (FIG. 38) suggesting that the OVCAR3-PT cells were more associated with an angiogenic phenotype than the OVCAR3 platinum sensitive/naïve cells.

To investigate if there was any specific chemokines driving the angiogenesis like phenotype in the OVCAR3-PT and OVCAR4-PT cells, we performed a cytokine array. This demonstrated increased expression of a number of cytokines that are key regulators of tumour angiogenesis (HGF, VEGF, TIMP1&2, PIGF and Angiogenin) in the PT resistant cells relative to the treatment-naïve control (FIG. 39). Since angiogenesis is largely driven by the expression of receptor tyrosine kinases, we analysed the gene expression profiling data from the HGSOC dataset and discovered a number of receptor tyrosine kinases (RTKs) which had higher expression levels in the AngioImmune subgroup (compared to the angio and immune groups) including PDGFRα (P=<0.0001), PDGFRβ (P=<0.0001), FGFR2 (P=0.008683), FGFR4 (P=0.008085), AXL (P=<0.0001), VEGFR1 (P=<0.0001), VEGFR2 (P=<0.0001), EGFR (P=0.04859), MET (P=0.02659), EPHA4 (P=0.00019), FZD1 (P=<0.0001). Interestingly PDGFRα (P=0.007074) and PDGFRβ (P=0.004868) were also associated with the post-platinum treated patient samples (FIG. 40). Additionally pRTK array was performed and demonstrated that the OVCAR3 platinum-resistant cells have a higher expression of key RTKs that are key regulators of downstream pathways that regulate tumour angiogenesis (FGFR2, FGFR3, AXL, VEGFR3, Insulin receptor, ERBb2), this was further validated by western blot (FIG. 41).

### Tyrosine Kinase Inhibitors (TKi) Selectively Target Platinum Resistant Cells

As many of the RTK were associated with the AngioImmune group and post-chemotherapy samples, we wanted to examine whether the OVCAR3-PT resistant cells would be sensitive to inhibitors of RTKs. We used 2 RTK inhibitors, Cediranib which targets VEGFR1-3 and PDGFRα/β and Nintedanib which targets VEGFR1-3, PDGFRα/β FGFR1-3 and performed 10-day colony formation assays. This demonstrated that Cediranib (VEGFR1-3 and PDGFRα/β inhibitor) and Nintedanib (VEGFR1-3, PDGFRα/β FGFR1-3 inhibitor) have specificity for the cisplatin-resistant OVCAR3 cells relative to the OVCAR3 cisplatin-naïve cell line (fold change 0.2390869 and 0.377, respectively) (FIG. 42). Additionally Cediranib and Nintedanib downregulated the expression of the VEGFR ligand, VEGFa, in the OVCAR3 and OVCAR4 cisplatin resistant cell lines. (FIG. 43) suggesting that the mechanism of sensitivity if through VEGFa. To examine the dependency of the angiogenesis phenotype on VEGFA, we performed an in-vivo matrigel plug assay where OVCAR3 cells in co-cultured with ECFCs and the mice were treated with 5 mg/kg of the VEGFRA inhibitor Bevacizumab. Quantification of MVD showed that the OVCAR3 cisplatin-resistant cells following treatment with bevacizumab (5 mg/kg) have less MVD in comparison to the OVCAR3 cisplatin-naïve cells (p-value: 0.0024) (FIG. 44). This demonstrates that the OVCAR3-PT cells (which have elevated 45 and 15 gene signature scores) are more sensitive to the VEGFA inhibitor Bevacizumab.

### The 45 and 15-Gene Signatures are Predictive of Response to Taxanes in Prostate Cancer

Within a pilot of 56 biopsy samples with de novo metastatic disease, 50 samples passed all QC metrics and were utilised for data analysis, 24 of which were PSA-responders and 26 non PSA-responders. It was observed that within the proportion of PSA responders, there was a significant increase in the 45-gene signature scores (p=0.0083) (FIG. 32a). Of these PSA-responders, 16 were 45-gene signature positive and 8 were 45-gene signature negative. Conversely to this, within the non PSA-responders 10 were 45-gene positive and 16 were 45-gene negative (FIG. 32c). EMT positive (45-gene signature positive) patients show a significant benefit to receiving taxane based chemotherapy with significantly increased overall survival in comparison to the EMT negative patients (HR 0.5094 [0.2694-0.9004]; p=0.0238 (FIG. 32b).

It was also observed that within the proportion of PSA responders, there was a significant increase in the 15-gene signature scores (p=0.04) (FIG. 33a). Of these PSA-responders, 16 were 15-gene signature positive and 8 were 15-gene signature negative. Conversely to this, within the non PSA-responders 10 were 15-gene positive and 16 were 15-gene negative (FIG. 33c). Additionally, EMT positive (15-gene signature positive) patients show a significant benefit to receiving taxane based chemotherapy with increased overall survival in comparison to the EMT negative patients (HR 0.66 [0.36-1.20]; p=0.160 (FIG. 33b).

### EMT Signature is Prognostic in Prostate Cancer, Predicting Disease Recurrence and Poor Prognosis Post Radical Surgery

In a retrospective cohort of 322 radical prostatectomy specimens, the EMT signature exhibits a prognostic relevance in Prostate Cancer. The EMT assay predicts biochemical disease recurrence whereby high EMT patients demonstrate a significantly poor prognostic group in comparison to low EMT patients (HR=1.8095 [1.1499-2.8474]; p=0.0658) (FIG. 34). Likewise, in a retrospective cohort of 248 radical radiotherapy specimens, the EMT signature also exhibits a prognostic relationship in Prostate Cancer. The EMT assay predicts both biochemical disease recurrence and metastatic disease progression whereby high EMT patients demonstrate a significantly poor prognostic group in comparison to low EMT patients (Biochemical: 2.4924 [1.4293-4.3463]; p=0.004 and Metastatic: HR=2.6351 [1.0998-6.3137]; p=0.020) (FIGS. 35a and b).

### Discussion

We have demonstrated that the MAPK pathway is a mechanism of innate and acquired resistance to SoC cisplatin-based therapy in HGSOC. The Angio_Immune group represents 44% of treatment naive HGSOCs and also represents a subgroup which samples migrate to after platinum-based chemotherapy treatment. At present all HGSOCs are treated the same however even though there are differences in prognosis on SoC platinum based therapy. The Angio_Immune group is a poor prognosis subgroup on SoC therapy and therefore represents an opportunity for treatment with more targeted and effective therapeutic agents. Importantly, the 45 and 15 EMT signatures which detect the Angio_Immune group can act as predictive assays for these targeted therapies and hence should predict response to therapeutics in any cancer disease setting. This is significant as these patients cannot be identified by a pathologist. The Angio_Immune group and the 45 and 15 gene signatures which detect the subgroup have a number of potential utilities.

### Predicting Cisplatin Resistant HGSOC

The Angio_Immune group represents a poor prognosis subgroup (FIGS. 1a & 1b) and the gene signatures which detect the subgroup are significant in SoC platinum based therapy in HGSOC for PFS and OS (FIGS. 1c and 11a). In line with this, this has been demonstrated across multiple cell line models also (FIGS. 2e and 11b). The Angio_Immune group represents a group which is selected for by SoC platinum based chemotherapy (FIG. 1d). This has also been demonstrated in ovarian cell line models which have been made cisplatin resistant in vitro and which have higher 45 and 15 signature scores (FIGS. 2 and 11c).

### Predicting Response to Inhibitors of the MAPK Pathway

We have demonstrated that the Angio_Immune group is driven by MAPK signalling using publically available and internal datasets and methods (FIGS. 3 and 4). The MAPK pathway is currently being targeted by numerous drugs from major Pharmaceutical companies (Table 5). We would predict that the 45 and 15 gene signatures which detect the Angio_Immune group would predicts response to agents targeting the MAPK pathway in human cancer. We have demonstrated that both the 45 and 15 gene signatures predict response to the MEK inhibitor Trametinib across multiple cell line models (FIGS. 3d and 12d). Additionally the combination of MAPK inhibitor with cisplatin may be a viable option for first line therapy or as a therapeutic strategy for cisplatin resistant disease. We have demonstrated this utility by generating novel HGSOC cell line models and shown increased sensitivity to cisplatin after MEK inhibition in the cisplatin resistant cells compared to cisplatin sensitive counterparts (FIG. 5b).

### Predicting Response to Inhibitors of the EMT Pathway

Since the Angio_Immune group is associated with increased expression of EMT pathway related genes (FIG. 7), we would predict that the 45 and 15 gene expression signatures detecting this group would predict response to agents targeting the EMT pathway. Additionally the cisplatin resistant cells generated in this study have elevated EMT signalling as demonstrated by increased expression of vimentin, N-cadherin and slug and decreased E-cadherin expression (FIG. 7). This suggests that the EMT phenotype is related to cisplatin resistance. This has been previously been documented in the literature (Ahmed et al., 2010; Haslehurst et al., 2012; Marchini et al., 2013). Table 7 highlights the major compounds in development which target the EMT pathway, predominantly AXL inhibitors.

### Predicting Response to Inhibitors of the SRC Pathway

We have demonstrated that the MAPK and SRC pathways act in parallel and that activation of the MAPK pathway may predict resistance to inhibitors of the SRC pathway. FIGS. 8 and 14 demonstrate that the 45 and 15 EMT signatures can predict resistance to SRC inhibitors in cell line models. This would be worthy of further investigation in clinical samples where patients have been treated with a SRC inhibitor. We would hypothesise that the 45 and 15 gene expression signatures would predict resistant to agents targeting the SRC pathway. Table 6 outlines the key Pharma players with SRC inhibitors currently in development for a range of relevant disease indications.

### Predicting Response to Anti-Angiogenic Agents

Our data suggest that after chemotherapy treatment, patient tumours move into the Angio_Immune group and therefore become more angiogenic like. The Angio_Immune subgroup is largely driven by angiogenic-like processed (FIG. 1). Therefore the use of anti-angiogenic agents should have better responses in the second line treatment of ovarian cancer following SoC cisplatin-based chemotherapy in the first line setting. Additionally we would hypothesise that since the Angio_Immune group is driven by Receptor Tyrsoine Kinases, the MAPK and EMT pathways and is increased angiogenesis signalling, the 45 and 15 gene signatures should predict response to any agent targeting the angiogenesis process. A number of anti-angiogenics agents are in clinical development, including bevacizumab, pazopanib, cedirinib, nintedanib, aflibercept, trebananib, sunitinib, sorafenib, and (PDGFR) imatinib.

Following a critical review of the various phase III anti-angiogenic clinical trials, there is a clear change in the biology of ovarian cancer following relapse and particularly on platinum resistance. There is a trend to a higher number of positive anti-angiogenic trials in platinum resistant ovarian cancer (see below). This confirms our hypothesis and suggests that the use of anti-angiogenic targeted agents may work better in second line trials after primary cisplatin treatment. The 45 and 15 EMT signatures should therefore predict response to these agents.

### Predicting Response to Taxanes

We have demonstrated that the EMT subgroup can predict response to taxane based chemotherapy. FIGS. 32 and 33 demonstrate that the 45 and 15 EMT signatures can predict sensitivity to taxanes in prostate cancer patients with de novo metastatic disease. This would be worthy of further investigation in clinical samples which incorporate a larger clinical validation cohort. We would hypothesise that the 45 and 15 gene expression signatures would predict response to taxane based chemotherapy whereby MEK positivity will be associated with increased overall survival.

### First-Line Treatment

There has been a number of phase II/III clinical trials focusing on target treatment with an aim to improve the overall survival of patients with high risk stage Ic and II-IV ovarian cancer. GOG 218 and ICON7 explored the role of bevacizumab (Avastin) in combination with upfront chemotherapy. Bevacizumab is a recombinant humanised monoclonal antibody that binds to vascular endothelial growth factor A (VEGF-A). ICON7 showed superior progression free survival (PFS) in the group of patients who received bevacizumab in combination with standard chemotherapy (20.3 months and 21.8 months, standard therapy versus standard therapy plus bevacizumab respectively (hazard ratio (HR) 0.81; 95% confidence interval (CI), 0.70 to 0.94; P=0.004)). Benefit was observed in patients with high risk disease (defined as FIGO III, ≥1.0 cm disease following debulking or FIGO stage IV).

GOG-218 similarly compared three arms; standard chemotherapy, bevacizumab with standard chemotherapy from cycle 2 to cycle 6 and bevacizumab plus standard chemotherapy cycle 2 through to cycle 22. There was again superior PFS when comparing standard chemotherapy and standard treatment with the addition of bevacizumab (10.3, 11.2, and 14.1 months, control group, bevacizumab (cycle 2-6), and bevacizumab (cylce2-22), respectively). The HR for PFS was 0.908; 95% CI, 0.795 to 1.040; P=0.16 for bevacizumab cycle 2-6 and 0.717; 95% CI, 0.625 to 0.824 (P<0.001) for bevacizumab plus chemotherapy from cycle 2-22.

### Recurrent/Relapsed Disease

As previously discussed the prognosis of ovarian cancer is directly correlated with platinum sensitivity and timing or recurrence or relapse following completion of platinum-based chemotherapy. Therefore, anti-angiogenics were explored in recurrent/relapsed ovarian cancer. Two phase III trials OCEANS and AURELIA explored the impact of bevacizumab in relation to the timing of disease relapse following platinum-based chemotherapy.

OCEANS is a phase III trial exploring the efficacy of bevacizumab in combination with gemcitabine and carboplatin (GC) in platinum sensitive recurrent ovarian cancer. Patients were assigned to bevacizumab+GC or placebo+GC, total number of six to ten cycles. Median progression free survival was 12.4 months and 8.4 months respectively (HR 0.484; 95% CI: 0.388-0.605; p-value<0.0001). The role of bevacizumab in platinum resistant epithelial ovarian cancer was explored in the AURELIA trial. In this phase III clinical trials patients were randomly assigned to single agent chemotherapy (investigators choice: peglated doxorubicin, paclitaxel or topotecan) with or without bevacizumab. Progression free survival as a primary endpoint was reached, 6.7 months in the chemotherapy and bevacizumab treated patients and 3.4 months in the chemotherapy alone arm (HA 0.42; 95% CI: 0.32-0.53; p-value<0.001). The objective response rate (ORR) in patients treated with bevacizumab and chemotherapy was 30.9%. Most importantly the benefit in PFS was reflected in the overall survival; 16.6 months in the chemotherapy and bevacizumab arm compared to 13.3 months in the chemotherapy alone arm (HR 0.89; 95% CI: 0.69-1.14; p-value<0.174). However this was not statistically significant. This trial led to the Food Drugs Advisory (FDA) approval of bevacizumab in platinum resistant epithelial ovarian cancer in November 2014, with an added benefit in relation to progression free survival in patients treated with bevacizumab and paclitaxel (PFS 9.6 months).

ICON6 was an international phase III clinical trial, testing the efficacy of cediranib, an oral potent inhibitor of VEGFR 1, 2 and 3 that as a direct effect in stopping the VEGF signal, in relapsed platinum sensitive epithelial ovarian cancer. Patients were randomised into three arms; chemotherapy plus placebo maintenance (reference arm), concurrent platinum chemotherapy and cediranib followed by maintenance cediranib or cediranib plus maintenance cediranib. There was statistically significant progression free and overall survival benefit (PFS 9.4 months and 11.4 months respectively; HR 0.68; p-value=0.0022, Overall survival 17.6 months and 20.3 months respectively; HR 0.70; p-value=0.0419).

### Predicting Poor Prognosis Group in Multiple Solid Tumours

Many cancers are driven by alterations in the MAPK pathway. For example mutations in the RAS genes (KRAS, HRAS, and NRAS), are present in approximately 50% of all patients with colorectal cancer. This results in hyper-activation of RAS proteins and their corresponding downstream pathways such as the MAPK pathway, thereby stimulating the development and progression of malignancy. We have demonstrated that both the 45 and 15 gene signatures predict poor prognosis subgroups in colon, lung and prostate cancer (FIGS. 9-10, 15-16, 32-35). Additionally the 15 gene signature was prognostic across a number of tissues (FIG. 36), suggesting that the EMT subgroup and the signatures which detect it (15 and 45 gene signatures) define a poor prognosis subgroup of patients across multiple diseases and is driven by the MAPK pathway activation and EMT signalling. We would hypothesise that this would be the situation for most solid tumours which have alterations in the MAPK or EMT pathways by mutation or mechanisms other than mutation which can activate the pathway.

### Example 2

### 15 Gene Signature Minimum and Core Gene Analysis

### Samples:

Internal training samples: This sample set comprised of 265 macro-dissected ovarian cancer FFPE tissue samples sourced from the Edinburgh Ovarian Cancer Database
Tothill samples: This is a publically available dataset, from which 278 Ovarian samples were used for analysis
ICON7 samples: This sample set comprised 140 SOC (Standard of Care) samples from a phase III randomized trial of carboplatin and paclitaxel with or without bevacizumab first line cancer treatment which were accessed through the MRC (Medical Research Council). The 140 samples used were patients who did not receive the addition of bevacizumab.

### Methods:

### Subtype Analysis

This analysis evaluates if the gene expression data for each of the signature genes has the ability to significantly detect the MEK subtype independent of the other genes. For each gene an area under the receiver operating curve (AUC) and ANOVA p-values were calculated in the internal training samples. Signature genes with an ANOVA p-value less than 0.05 are statistically significant at detecting the subtype, independent of the other signature genes. C-index values were also generated to determine if the gene expression for each of the signature genes has the ability to significantly detect PFS (Progression Free Survival) independent of the other signature genes. The upper and lower confidence intervals (CI) were derived using bootstrapping with 1000 samplings. If the C-Index lower CI is greater than 0.5 or the upper CI is less than 0.5 then the C-index indicates that the gene expression is significantly associated to the observed survival.

### Core Gene Analysis

The purpose of evaluating the core gene set of the signature is to determine a ranking for the genes based upon their impact on performance when removed from the signature.

This analysis involved 10,000 random samplings of 10 signature genes from the original 15 signature gene set. At each iteration, 10 randomly selected signature genes were removed and the performance of the remaining 5 genes was evaluated using the endpoint to determine the impact on HR (Hazard Ratio) performance when these 10 genes were removed in the following 2 datasets:
Tothill—278 samples
ICON7 SOC (Standard of Care)—140 samples

ICON7 was evaluated using the PFS endpoint and Tothill was evaluated using the OS (Overall Survival) endpoint. Within each of the 2 datasets, the signature genes were weighted based upon the change in HR performance (Delta HR) based upon their inclusion or exclusion. Genes ranked ‘1’ have the most negative impact on performance when removed and those ranked ‘15’ have the least impact on performance when removed.

### Minimum Gene Analysis

The purpose of evaluating the minimum number of genes is to determine if significant performance can be achieved within smaller subsets of the original signature.

This analysis involved 10,000 random samplings of the 15 signature genes starting at 1 gene/feature, up to a maximum of 10 genes/features. For each randomly selected feature length, the signature was redeveloped using the PLS machine learning method under CV and model parameters derived. At each feature length, all randomly selected signatures were applied to calculate signature scores for the following 2 datasets:
Tothill—278 samples
ICON7 SOC (Standard of Care)—140 samples

Continuous signature scores were evaluated with outcome to determine the HR effect; ICON7 was evaluated with PFS and Tothill was evaluated with OS. The HR for all random signatures at each feature length was summarized and figures generated to visualize the performance over CV.

### Results

### Subtype Analysis

The results for the subset analysis of the 15 gene signature in the internal training dataset is provided in this section.
AUC and ANOVA: All genes have a p value significantly less than the 0.05 threshold for ANOVA (See Table 12). This suggests that every individual gene in the 15 gene signature has the ability to detect the subtype independent of the other 14 genes.
C-Index: All genes have expression values that significantly discriminated the survival probability of the patients (See Table 12). The C-Index values for each of these genes is higher than 0.5 therefore indicating that lower expression correlates to higher survival probability for all 15 genes.

### Core Gene Analysis

The results for the core gene analysis of the 15 gene signature in the 2 datasets is provided in this section.
Tothill: Delta HR performance measured in this dataset for the 15 signature genes is shown in FIG. 22. This figure highlights the top 10 ranked genes in the signature which are the most important in retaining a good HR performance within this dataset.
ICON7: Delta HR performance measured in this dataset for the 15 signature genes is shown in FIG. 23. This figure highlights the top 10 ranked genes in the signature which are the most important in retaining a good HR performance within this dataset.
Delta HR across these 2 datasets was evaluated to obtain a combined gene ranking for each of the signature genes. The ranks assigned to the signature genes based on the core set analysis has been outlined in Table B.

### Minimum Gene Analysis

The results for the minimum gene analysis of the 15 gene signature in 2 datasets is provided in this section.
Tothill: The average HR performance measured in this dataset using the random sampling of the signature genes from a feature length of 1 to 10 is shown in FIG. 24. This figure shows that to retain a significant HR performance (i.e. HR<1) a minimum of 1 of the signature genes must be selected.
ICON7 SOC: The average HR performance measured in this dataset using the random sampling of the signature genes from a feature length of 1 to 10 is shown in FIG. 25. This figure shows that to retain a significant HR performance (i.e. HR<1) a minimum of 1 of the signature genes must be selected.
In summary, it is recommended that a minimum of at least 1 gene can be used and significant performance will be retained.

### Example 3

### 45 Gene Signature Minimum and Core Gene Analysis

### Samples:

Internal training samples: This sample set comprised of 265 macro-dissected ovarian cancer FFPE tissue samples sourced from the Edinburgh Ovarian Cancer Database
Tothill samples: This is a publically available dataset, from which 278 Ovarian samples were used for analysis
ICON7 samples: This sample set comprised 140 SOC (Standard of Care) samples from a phase III randomized trial of carboplatin and paclitaxel with or without bevacizumab first line cancer treatment which were accessed through the MRC (Medical Research Council). The 140 samples used were patients who did not receive the addition of bevacizumab.

### Methods:

### Subtype Analysis

This analysis evaluates if the gene expression data for each of the signature genes has the ability to significantly detect the MEK subtype independent of the other genes. For each gene an area under the receiver operating curve (AUC) and ANOVA p-values were calculated in the internal training samples. Signature genes with an ANOVA p-value less than 0.05 are statistically significant at detecting the subtype, independent of the other signature genes. C-index values were also generated to determine if the gene expression for each of the signature genes has the ability to significantly detect PFS (Progression Free Survival) independent of the other signature genes. The upper and lower confidence intervals (CI) were derived using bootstrapping with 1000 samplings. If the C-Index lower CI is greater than 0.5 or the upper CI is less than 0.5 then the C-index indicates that the gene expression is significantly associated to the observed survival.

### Core Gene Analysis

The purpose of evaluating the core gene set of the signature is to determine a ranking for the genes based upon their impact on performance when removed from the signature.

This analysis involved 10,000 random samplings of 10 signature genes from the original 45 signature gene set. At each iteration, 10 randomly selected signature genes were removed and the performance of the remaining 35 genes was evaluated using the endpoint to determine the impact on HR (Hazard Ratio) performance when these 10 genes were removed in the following 2 datasets:
Tothill—278 samples
ICON7 SOC (Standard of Care)—140 samples

ICON7 was evaluated using the PFS endpoint and Tothill was evaluated using the OS (Overall Survival) endpoint. Within each of the 2 datasets, the signature genes were weighted based upon the change in HR performance (Delta HR) based upon their inclusion or exclusion. Genes ranked ‘1’ have the most negative impact on performance when removed and those ranked ‘45’ have the least impact on performance when removed.

### Minimum Gene Analysis

The purpose of evaluating the minimum number of genes is to determine if significant performance can be achieved within smaller subsets of the original signature.

This analysis involved 10,000 random samplings of the 45 signature genes starting at 1 gene/feature, up to a maximum of 20 genes/features. For each randomly selected feature length, the signature was redeveloped using the PLS machine learning method under CV and model parameters derived. At each feature length, all randomly selected signatures were applied to calculate signature scores for the following 2 datasets:
Tothill—278 samples
ICON7 SOC (Standard of Care)—140 samples

Continuous signature scores were evaluated with outcome to determine the HR effect; ICON7 was evaluated with PFS and Tothill was evaluated with OS. The HR for all random signatures at each feature length was summarized and figures generated to visualize the performance over CV.

### Results

### Subtype Analysis

The results for the subset analysis of the 45 gene signature in the internal training dataset is provided in this section.
AUC and ANOVA: All genes have a p value significantly less than the 0.05 threshold for ANOVA (See Table 13). This suggests that every individual gene in the 45 gene signature has the ability to detect the subtype independent of the other 44 genes.
C-Index: 38 of the 45 genes have expression values that significantly discriminate the survival probability of the patients (See Table 13). The C-Index values for each of these genes is higher than 0.5 therefore indicating that lower expression correlates to higher survival probability for the 38 genes.

### Core Gene Analysis

The results for the core gene analysis of the 45 gene signature in the 2 datasets is provided in this section.
Tothill: Delta HR performance measured in this dataset for the 45 signature genes is shown in FIG. 26 (which is also represented as Table 14). This figure highlights the top 10 ranked genes in the signature which are the most important in retaining a good HR performance within this dataset.
ICON7: Delta HR performance measured in this dataset for the 45 signature genes is shown in FIG. 27 (which is also represented as Table 15). This figure highlights the top 10 ranked genes in the signature which are the most important in retaining a good HR performance within this dataset.
Delta HR across these 2 datasets was evaluated to obtain a combined gene ranking for each of the signature genes. The ranks assigned to the signature genes based on the core set analysis has been outlined in Table A.

### Minimum Gene Analysis

The results for the minimum gene analysis of the 45 gene signature in 2 datasets is provided in this section.
Tothill: The average HR performance measured in this dataset using the random sampling of the signature genes from a feature length of 1 to 20 is shown in FIG. 28. This figure shows that to retain a significant HR performance (i.e. HR<1) a minimum of 2 of the signature genes must be selected.
ICON7 SOC: The average HR performance measured in this dataset using the random sampling of the signature genes from a feature length of 1 to 20 is shown in FIG. 29. This figure shows that to retain a significant HR performance (i.e. HR<1) a minimum of 1 of the signature genes must be selected.

### Tables

TABLE 1
Number of dataset overlapping genes defined as high ranking
by combined average variance-intensity in each disease indication

| Disease Indication | # High Ranked Genes |
|---|---|
| Ovarian | 14507 |
| Colon | 13151 |
| Lung | 13422 |
| Melanoma | 10820 |

TABLE 2
List of Entrez gene IDs, gene symbols and description for the
15 gene signature which was selected to predict the MEK subtype

| Entrez Gene ID | Gene Symbol | Description |
|---|---|---|
| 1009 | CDH11 | cadherin 11, type 2, OB-cadherin |
| | | (osteoblast) |
| 11031 | RAB31 | RAB31, member RAS oncogene family |
| 1289 | COL5A1 | collagen, type V, alpha 1 |
| 1300 | COL10A1 | collagen, type X, alpha 1 |
| 1462 | VCAN | versican |
| 2191 | FAP | fibroblast activation protein, alpha |
| 2335 | FN1 | fibronectin 1 |
| 23452 | ANGPTL2 | angiopoietin-like 2 |
| 2706 | GJB2 | gap junction protein, beta 2, 26 kDa |
| 3624 | INHBA | inhibin, beta A |
| 4323 | MMP14 | matrix metallopeptidase 14 |
| 5328 | PLAU | plasminogen activator, urokinase |
| 7057 | THBS1 | thrombospondin 1 |
| 7058 | THBS2 | thrombospondin 2 |
| 9945 | GFPT2 | glutamine-fructose-6-phosphate |
| | | transaminase 2 |

TABLE 3
The top 20 GO biological processes and GO terms from
functional enrichment analysis of the 15-gene signature

| | | | P-value |
|---|---|---|---|
| # | ID | Gene Ontology biological process | (FDR) |
| 1 | GO: 0044243 | multicellular organismal catabolic | 8.68E−07 |
| | | process | |
| 2 | GO: 0022610 | biological adhesion | 9.26E−07 |
| 3 | GO: 0007155 | cell adhesion | 9.11E−07 |
| 4 | GO: 0034329 | cell junction assembly | 8.21E−07 |
| 5 | GO: 0043062 | extracellular structure organization | 6.51E−07 |
| 6 | GO: 0030198 | extracellular matrix organization | 6.51E−07 |
| 7 | GO: 0034330 | cell junction organization | 1.46E−06 |
| 8 | GO: 0072359 | circulatory system development | 4.65E−06 |
| 9 | GO: 0072358 | cardiovascular system development | 4.65E−06 |
| 10 | GO: 0022617 | extracellular matrix disassembly | 4.71E−06 |
| 11 | GO: 0001568 | blood vessel development | 5.55E−06 |
| 12 | GO: 0044236 | multicellular organismal metabolic | 6.06E−06 |
| | | process | |
| 13 | GO: 0001944 | vasculature development | 7.34E−06 |
| 14 | GO: 0007160 | cell-matrix adhesion | 1.41E−05 |
| 15 | GO: 0001525 | angiogenesis | 2.09E−05 |
| 16 | GO: 0045216 | cell-cell junction organization | 2.55E−05 |
| 17 | GO: 0009653 | anatomical structure morphogenesis | 3.04E−05 |
| 18 | GO: 0007044 | cell-substrate junction assembly | 3.41E−05 |
| 19 | GO: 0051895 | negative regulation of focal adhesion | 3.94E−05 |
| | | assembly | |
| 20 | GO: 0001952 | regulation of cell-matrix adhesion | 3.56E−05 |

TABLE 4
A) Weighting and bias breakdown for each probeset within the
45-gene signature. B) Weighting and bias breakdown for each
probeset within the 15-gene signature.
a.
45 Gene Signature

| Rank | Gene Name | Weight | Bias |
|---|---|---|---|
| 1 | TMEM200A | 0.059481295 | 3.681329367 |
| 2 | GJB2 | 0.055985433 | 4.479833955 |
| 3 | MMP13 | 0.038284076 | 3.724107067 |
| 4 | GFPT2 | 0.037990641 | 4.860237265 |
| 5 | POSTN | −0.035480409 | 4.359882019 |
| 6 | BICC1 | 0.030426737 | 3.698203663 |
| 7 | CDH11 | 0.028340142 | 4.996780524 |
| 8 | MRVI1 | 0.025598535 | 5.076083782 |
| 9 | PMP22 | 0.024034610 | 5.564463361 |
| 10 | COL11A1 | −0.023672753 | 3.500170591 |
| 11 | IGFL2 | 0.022225316 | 3.310383445 |
| 12 | LUM | −0.022014619 | 8.336273473 |
| 13 | NTM | −0.021750365 | 4.230245127 |
| 14 | BGN | 0.021089508 | 10.15236225 |
| 15 | COL3A1 | −0.021023256 | 8.323635399 |
| 16 | COL10A1 | 0.019650845 | 6.353832828 |
| 17 | RAB31 | 0.018014921 | 5.317119481 |
| 18 | ANGPTL2 | 0.016630934 | 5.639562288 |
| 19 | PLAU | 0.016596202 | 5.848820224 |
| 20 | COL8A1 | 0.016373799 | 6.419330171 |
| 21 | MIR1245 | 0.015290888 | 5.455187262 |
| 22 | POLD2 | 0.014555548 | 9.38782491 |
| 23 | NKD2 | 0.014468847 | 7.371707677 |
| 24 | FZD1 | 0.014334768 | 4.151874676 |
| 25 | COPZ2 | 0.013866713 | 5.103944696 |
| 26 | ITGA5 | 0.013561913 | 8.36627973 |
| 27 | VGLL3 | 0.012488674 | 4.501866677 |
| 28 | INHBA | −0.011763261 | 4.684272993 |
| 29 | MMP14 | 0.011010832 | 10.08406264 |
| 30 | VCAN | 0.009977966 | 5.551759846 |
| 31 | THBS2 | −0.008700202 | 8.130920944 |
| 32 | RUNX2 | 0.008333275 | 4.73450528 |
| 33 | TIMP3 | 0.008141253 | 6.498316457 |
| 34 | SFRP2 | −0.007890741 | 5.601725816 |
| 35 | COL1A2 | 0.007788938 | 6.01000198 |
| 36 | COL5A2 | −0.007217722 | 3.567060064 |
| 37 | SERPINF1 | 0.006801251 | 10.8333948 |
| 38 | KIF26B | −0.005249312 | 4.97815094 |
| 39 | TNFAIP6 | 0.004963450 | 5.361760185 |
| 40 | MMP2 | 0.003988003 | 5.362247865 |
| 41 | FN1 | 0.003130435 | 4.984016427 |
| 42 | ALPK2 | 0.002394440 | 3.513604572 |
| 43 | CTSK | 0.001542586 | 5.732155915 |
| 44 | LOXL1 | −0.001415170 | 9.593869933 |
| 45 | FAP | −0.000007237 | 5.23E+00 |

b.
15 Gene Signature

| Rank | Gene Name | Entrez Gene ID | Weight | Bias |
|---|---|---|---|---|
| 1 | GJB2 | 2706 | 0.089719778 | 4.478098614 |
| 2 | CDH11 | 1009 | 0.066544238 | 4.990055702 |
| 3 | GFPT2 | 9945 | 0.058421032 | 4.885349473 |
| 4 | COL10A1 | 1300 | 0.040148445 | 6.357258041 |
| 5 | ANGPTL2 | 23452 | 0.038272311 | 5.631697532 |
| 6 | THBS1 | 7057 | 0.036613387 | 6.428114883 |
| 7 | RAB31 | 11031 | 0.033158407 | 5.300536304 |
| 8 | THBS2 | 7058 | −0.030849235 | 8.135441538 |
| 9 | INHBA | 3624 | −0.028500708 | 4.68290899 |
| 10 | MMP14 | 4323 | 0.020727894 | 10.07844987 |
| 11 | VCAN | 1462 | 0.020706504 | 5.529961284 |
| 12 | PLAU | 5328 | 0.019342831 | 5.850016491 |
| 13 | COL5A1 | 1289 | 0.010674165 | 5.569094517 |
| 14 | FAP | 2191 | −0.006101691 | 5.226391586 |
| 15 | FN1 | 2335 | −0.005998124 | 4.982941989 |

TABLE 5
Key Pharma players with major MEK inhibitor compounds which the 45-gene and 15-
gene signatures could predict response for.
A) RAS/RAF/MEK/ERK Inhibitors - Phase II/III Marketed

| DRUG NAME | COMPANY NAME | DISEASE INDICATION |
|---|---|---|
| vemurafenib | Roche | Marketed: mMelanoma |
| (Zelboraf) | | Phase II: Bile Duct, Bladder, CLL, GI, |
| | | Leukemia, Ovarian, Prostate, Sarcomas, |
| | | Thyroid |
| regorafenib | Bayer | Marketed: GIST, mCRC |
| (Stivarga) | | PhIII: HCC |
| | | PhII: Bile Duct, Ovarian, Pancreatic, RCC, |
| | | Salivary Gland, Soft Tissue Sarcoma; Bladder |
| dabrafenib | Novartis | Marketed: mMelanoma |
| (Tafinlar) | | PhII: Thyroid, CRC, NSCLC, (GIST), Glioma, |
| | | Leukemia, Brain, Multiple Myeloma, Germ |
| | | Cell Tumors |
| RAF-265 | Novartis | PhII: mMelanoma |
| encorafenib | Array Biopharma | PhIII: mMelanoma |
| | | PhII: mCRC |
| donafenib | Suzhou Zelgen | PhII: Esophageal, GI, HCC, mCRC |
| | Biopharmaceutical | |
| NEO-100 | Neonc | PhII: Recurrent Glioblastoma Multiforme |
| | Technologies | (GBM) |
| | | Preclinical: Lung |
| PLX-8394 | Plexxikon | phII: Thyroid, Bile Duct, CRC, Melanoma, |
| | | NSCLC |
| | | phI: Leukemias |
| RXDX-105 | Ingnyta | PhII: Colon Carcinoma, Melanoma, mCRC |
| TAK-580 | Millennium | PhII: Metastatic Melanoma; Solid Tumor |
| | Pharmaceuticals | PhI: Nonhematologic Malignancy |
| ulixertinib | BioMed Valley | PhII: AML, CRC, Melanoma, Myelodysplastic |
| | Discoveries | Syndrome; NSCLC |
| | | Preclinical: Metastatic Adenocarcinoma of |
| | | The Pancreas; Pancreatic |

B) RAS/RAF/MEK/ERK Inhibitors - Phase I, Pre-Clinical

| DRUG NAME | COMPANY |
|---|---|
| BAL-3833 | Basilea Pharmaceutica |
| BGB-283 | Merck KGaA |
| HM-95573 | Hanmi Pharmaceuticals |
| LY-3009120 | Eli Lilly |
| RG-7304 | Roche |
| RG-7842 | Genentech |
| salirasib | Ono Pharmaceutical |
| AEZS-136 | Aeterna Zentaris Inc. |
| ARI-4175 | Arisaph Pharmaceuticals |
| ASN-003 | Asana BioSciences |
| CCT-196969 | Basilea Pharmaceutica |
| CCT-241161 | Basilea Pharmaceutica |
| CS-410 | Chipscreen Biosciences |
| Small Molecule to | Genentech, Inc. |
| Inhibit MAP4K4 | |
| for Cancer | |
| Small Molecules | Novartis AG |
| to Inhibit pan- | |
| RAF Kinase for | |
| Oncology | |
| CT-207 | HEC Pharm Co., Ltd. |
| CT-317 | HEC Pharm Co., Ltd. |
| Drugs to Inhibit B- | Ruga Corporation |
| Raf Kinase | |
| for Cancer | |
| EBI-907 | Eternity Bioscience Inc. |
| EBI-945 | Eternity Bioscience Inc. |
| KO-947 | Kura Oncology, Inc. |
| LXH-254 | Novartis AG |
| MDC-1016 | Medicon Pharmaceuticals, |
| | Inc |
| MT-477 | Medisyn Technologies, Inc. |
| NCB-0594 | Carna Biosciences, Inc. |
| NCB-0846 | Carna Biosciences, Inc. |
| NMSP-285 | Nerviano Medical Sciences |
| | S.r.l. |
| ON-108600 | Onconova Therapeutics, Inc. |
| PV-103 | PepVax, Inc. |
| RX-8243 | Rexahn Pharmaceuticals, |
| | Inc. |
| STP-503 | Sirnaomics, Inc. |
| BAL-3833 | Basilea Pharmaceutica |
| BGB-283 | Merck KGaA |
| HM-95573 | Hanmi Pharmaceuticals |
| LY-3009120 | Eli Lilly |
| RG-7304 | Roche |
| RG-7842 | Genentech |
| salirasib | Ono Pharmaceutical |
| AEZS-136 | Aeterna Zentaris Inc. |
| ARI-4175 | Arisaph Pharmaceuticals |
| ASN-003 | Asana BioSciences |
| CCT-196969 | Basilea Pharmaceutica |
| CCT-241161 | Basilea Pharmaceutica |
| CS-410 | Chipscreen Biosciences |
| Small Molecule to | Genentech, Inc. |
| Inhibit MAP4K4 | |
| for Cancer | |
| Small Molecules to | Novartis AG |
| Inhibit pan-RAF | |
| Kinase for | |
| Oncology | |
| CT-207 | HEC Pharm Co., Ltd. |
| CT-317 | HEC Pharm Co., Ltd. |
| Drugs to Inhibit B- | Ruga Corporation |
| Raf Kinase | |
| for Cancer | |
| EBI-907 | Eternity Bioscience Inc. |
| EBI-945 | Eternity Bioscience Inc. |
| KO-947 | Kura Oncology, Inc. |
| LXH-254 | Novartis AG |
| MDC-1016 | Medicon Pharmaceuticals, Inc |
| MT-477 | Medisyn Technologies, Inc. |
| NCB-0594 | Carna Biosciences, Inc. |
| NCB-0846 | Carna Biosciences, Inc. |
| NMSP-285 | Nerviano Medical Sciences |
| | S.r.l. |
| ON-108600 | Onconova Therapeutics, Inc. |
| PV-103 | PepVax, Inc. |
| RX-8243 | Rexahn Pharmaceuticals, Inc. |
| STP-503 | Sirnaomics, Inc. |

TABLE 6
Key Pharma players with major SRC inhibitor compounds which
the 45-gene and 15-gene signatures could predict response for.

| | | | PRODUCT |
|---|---|---|---|
| DRUG NAME | COMPANY | INDICATION | STAGE |
| ilorasertib | AbbVie | Solid Tumor | Ph II |
| | | Ovarian | Pre-clinical |
| KX-01 | Athenex | Leukemia | PhI |
| pazopanib | GSK | mRCC | PhII |
| hydrochloride + | | | |
| pembrolizumab | | | |
| tesevatinib | Kadmon | mBrain, mBreast, | PhII |
| tosylate | Corporation | NSCLC | |
| VAL-201 | ValiRx Plc | Breast, mProstate, | PhII |
| | | Ovarian | |
| AZD-0424 | AZ | Oncology | PhI |
| BGB-102 | BeiGene(Beijing) | Oncology | PhI |
| KX-02 | Athenex, Inc. | Brain, Lymphoma | PhI |
| rebastinib tosylate | Deciphera | Leukemia | PhI |
| | Pharmaceuticals | mBreast | Pre-clinical |
| ASN-006 | Asana BioSciences | Oncology | Pre-clinical |
| CCT-196969 | Basilea | Melanoma | Pre-clinical |
| CCT-241161 | Pharmaceutica AG | | |
| ORB-0001 | OriBase Pharma | Leukemia | Pre-clinical |
| Misc | MI.TO. Technology | Oncology | Pre-clinical |
| | S.r.L. | | |
| Misc | University of | Prostate | Pre-clinical |
| | Toledo | | |
| RK-20449 | Riken Advanced | Leukemia | Pre-clinical |
| | Science Institute | | |

Various others in Discovery stage by Academic institutions

TABLE 7
Key Pharma players with major EMT inhibitor compounds, mainly AXL inhibitors,
which the 45-gene and 15-gene signatures could predict response for.

| DRUG NAME | COMPANY | INDICATION | PRODUCT STAGE |
|---|---|---|---|
| gilteritinib | Astellas | NSCLC | Ph III |
| fumarate | | Leukemia | Ph II |
| MGCD-265 | Mirati Therapeutics | Head & Neck, NSCLC | Ph II |
| MGCD-516 | Mirati Therapeutics | Lung | Ph I |
| S-49076 | Servier | NSCLC, Brain | Ph II |
| | | Colon, HCC | Pre-clinical |
| BPI-9016 | Zhejiang | Gastic, Liver, Lung | Ph I |
| | BetaPharma | | |
| CT-053 | EC Pharm Co., Ltd. | Brain | Ph I |
| | | Bladder, Breast, HCC, RCC, Lung, | Pre-clinical |
| | | Ovarian | |
| BGB-324 | BerGenBio | Leukemia, Lung | Ph I |
| | | Breast, Pancreatic | Pre-clinical |
| BGB-10C9 | BerGenBio | Pancreatic | Pre-clinical |
| Misc AXLi | BerGenBio | Oncology | Pre-clinical |
| HuMax-AXL- | GenMab | Solid Tumor | Pre-clinical |
| ADC | | | |
| LDC-2636 | Lead Discovery | Leukemia | Pre-clinical |
| | Center | | |
| Misc AXLi | Protelica | Oncology | Pre-clinical |
| | Incorporated | | |
| NPS-1034 | NeoPharm | NSCLC | Pre-clinical |
| Q-701 | Clurient Co., Ltd. | Oncology | Pre-clinical |
| RXDX-106 | Ignyta, Inc. | Leukemia, Breast, GI, Melanoma; | Pre-clinical |
| | | mCRC, NSCLC, Ovarian, Pancreatic | |
| SGI-7079 | Tolero | NSCLC | Pre-clinical |
| | Pharmaceuticals, | | |
| | Inc. | | |
| TP-0903 | Tolero | Leukemia; Head And Neck, Lung, | Pre-clinical |
| | Pharmaceuticals, | Pancreatic | |
| | Inc. | | |
| Misc AXLi | SignalChem | Oncology | Pre-clinical |
| | Lifesciences | | |
| Misc AXLi | University of | NSCLC | Pre-clinical |
| | Colorado | | |
| Misc AXLi | Kolltan | Oncology | Discovery |
| | Pharmaceuticals | | |

TABLE 8
EMT gene analysis: Angio_immune/MEK/EMT group vs Rest

| | AUC | |
|---|---|---|
| EMT related gene | (CI) | P VALUE |
| VIMENTIN | 0.6706 | P = <0.0001 |
| | (0.6053 to 0.7359) | |
| AXL RTK | 0.6637 | P = <0.0001 |
| | 0.5971 to 0.7304 | |
| TWIST1 | 0.7674 | P = <0.0001 |
| | (0.7096 to 0.8252) | |
| SNAIL | 0.5748 | P = 0.03922 |
| | (0.5144 to 0.6452) | |
| SLUG | 0.7998 | P = <0.0001 |
| | (0.7471 to 0.8525) | |

TABLE 9
Up-regulated and Down-regulated genes in the Angio-Immune subgroup
AngioImmune

| | Down- |
|---|---|
| Up-regulated | regulated |
| RASGRF2 | RBFOX1 |
| COL1A2 | FNBP1 |
| GDF6 | GABRG2 |
| CNTN1 | AZGP1 |
| BCHE | SLCO1B1 |
| HAND2-AS1 | RBM24 |
| FABP4 | GPR98 |
| GDF6 | SMCO3 |
| EFEMP1 | PTPRT |
| ADIPOQ | IGSF1 |
| SPATS2L | LINC00240 |
| GRP | RMST |
| EFEMP1 | MYB |
| PDLIM5 | CYP19A1 |
| MEGF10 | CYP19A1 |
| ZFHX4 | GNAO1 |
| PTPRC | NPY6R |
| THSD7A | ENDOD1 |
| UCA1 | RMST |
| ADH1B | GJB7 |
| PRKG1 | TPTE2P5 |
| CRISPLD2 | FAM124A |
| GLRX | FCGBP |
| LRRN4CL | SH3GL2 |
| DEPDC7 | DBF4 |
| PIEZO2 | MYH6 |
| HOXA5 | SLC6A15 |
| GFRA1 | PKHD1L1 |
| FYB | SYBU |
| HS3ST3B1 | C11orf70 |
| FMO2 | TPRG1 |
| BEND6 | ZNF98 |
| PPP1R3B | RNF39 |
| GALNT15 | C6orf183 |
| CBLN4 | GLDN |
| ADH1B | OR52L1 |
| TBX5 | CYP4A11 |
| PAPPA | CACHD1 |
| TFEC | CHRM2 |
| C4orf32 | SMARCE1P2 |
| BNC1 | EPHA6 |
| MFAP5 | HIBCH |
| CALB1 | RFX3 |
| MCTP1 | |
| CDH13 | |
| HOXA5 | |
| FEM1B | |
| TDO2 | |
| UCA1 | |
| RUNX1T1 | |
| IGHG3 | |
| CHSY3 | |
| ELN | |
| RAB30 | |
| C1QTNF7 | |
| CBLC | |
| CD36 | |
| MDFIC | |
| TENM3 | |
| LSAMP | |
| TAGAP | |
| LMOD1 | |
| HRH1 | |
| FPR3 | |
| GAPDHP59 | |
| GALNT5 | |
| PDE1A | |
| EMCN | |
| STEAP4 | |
| IL18R1 | |
| CXCL9 | |
| DOCK8 | |
| ASIC2 | |
| HMCN1 | |
| CAMK2D | |
| SPATA9 | |
| CD2 | |
| MDFIC | |
| CXCL5 | |
| FBXO32 | |
| ARHGAP42 | |
| IFNG-AS1 | |
| LCP2 | |
| SLC7A5 | |
| PLIN1 | |
| COL12A1 | |
| P2RY8 | |
| CCR1 | |
| DLX5 | |
| ADAMTS5 | |
| SNORD114-7 | |
| CHRNA1 | |
| TLR1 | |
| NR4A1 | |
| GLRX | |
| DCLK1 | |
| PDE1A | |
| NR4A3 | |
| MMP1 | |
| LRRK2 | |
| CELF2 | |
| GUCY1A3 | |
| SLAMF7 | |
| BIRC3 | |
| DPP4 | |
| ADH1B | |
| SIGLEC10 | |
| LRRK2 | |
| CRLS1 | |
| TFEC | |
| CLEC1A | |
| ADRA2A | |
| NFATC2 | |
| SATB2 | |
| RGCC | |
| IL6 | |
| WNT4 | |
| HIVEP3 | |
| SDC2 | |
| JAKMIP1 | |
| GPR34 | |
| PRELP | |
| HAND2-AS1 | |
| SHOX2 | |
| GPC6 | |
| TSPAN5 | |
| TNFRSF11A | |
| ALOX5AP | |
| SNORD114- | |
| 31 | |
| RUNX1T1 | |
| HGF | |
| TFPI2 | |
| CHRNA1 | |
| AOX1 | |
| PDE1A | |
| ANGPTL2 | |
| FBXO40 | |
| FCRL6 | |
| NAMPT | |
| MS4A6A | |
| MPP4 | |
| PNMA2 | |
| DCLK1 | |
| CAMK2D | |
| ITGB3 | |
| FAM129A | |
| GCNT4 | |
| NPAS2 | |
| STAP1 | |
| MEGF10 | |
| FOSL2 | |
| BNIP3L | |
| SLC22A3 | |
| TNNI2 | |
| SNORD114-7 | |
| SLC24A3 | |
| PSTPIP2 | |
| PLA2R1 | |
| PRDM6 | |
| HGF | |
| AOX1 | |
| HRH1 | |
| BNC2 | |
| LINC00842 | |
| IGJ | |
| ERCC1 | |
| RFX2 | |
| RARRES1 | |
| CPXM2 | |
| ATP11A | |
| BAG2 | |
| IRF4 | |
| SKAP2 | |
| UBAC2 | |
| ADAMTS9- | |
| AS2 | |
| SEMA5A | |
| BMP2 | |
| FOXO1 | |
| LINC00626 | |
| NKD1 | |
| ZMYM5 | |
| NPTX1 | |
| NR4A3 | |

TABLE 10
Angio_Immune molecular subtype gene expression and function data

| Directionality | Group | Description | Query.size | Population | Target.size | Intersection | Pvalue | Pvalue.fdr | Genes |
|---|---|---|---|---|---|---|---|---|---|
| upregulated | GO: 0048870 | cell motility | 139 | 15462 | 978 | 30 | 2.24E−09 | 2.25E−06 | GPC6, DCLK1, SOS1, SEMA5A, |
| | | | | | | | | | ADRA2A, CD2, HOXA5, NFATC2, MMP1, |
| | | | | | | | | | SATB2, LRRK2, NKD1, NR4A1, PRKG1, |
| | | | | | | | | | CDH13, DPP4, CCR1, COL1A2, PTPRC, |
| | | | | | | | | | BMP2, RGCC, TNFRSF11A, ITGB3, |
| | | | | | | | | | WNT4, CXCL5, IL6, TBX5, SLC7A5, CXCL9, ADIPOQ |
| upregulated | GO: 0051674 | localization of | 139 | 15462 | 978 | 30 | 2.24E−09 | 2.25E−06 | GPC6, DCLK1, SOS1, SEMA5A, |
| | | cell | | | | | | | ADRA2A, CD2, HOXA5, NFATC2, MMP1, |
| | | | | | | | | | SATB2, LRRK2, NKD1, NR4A1, PRKG1, |
| | | | | | | | | | CDH13, DPP4, CCR1, COL1A2, PTPRC, |
| | | | | | | | | | BMP2, RGCC, TNFRSF11A, ITGB3, |
| | | | | | | | | | WNT4, CXCL5, IL6, TBX5, SLC7A5, CXCL9, ADIPOQ |
| upregulated | GO: 0040011 | locomotion | 139 | 15462 | 1296 | 35 | 2.25E−09 | 2.25E−06 | GPC6, DCLK1, ADRA2A, CD2, HOXA5, |
| | | | | | | | | | NFATC2, NKD1, GFRA1, DPP4, COL1A2, |
| | | | | | | | | | ITGB3, RGCC, CXCL9, CNTN1, NR4A3, |
| | | | | | | | | | FPR3, SEMA5A, SOS1, SATB2, MMP1, |
| | | | | | | | | | LRRK2, NR4A1, PRKG1, DLX5, CDH13, |
| | | | | | | | | | PTPRC, BMP2, CCR1, TNFRSF11A, |
| | | | | | | | | | CXCL5, WNT4, IL6, TBX5, SLC7A5, ADIPOQ |
| upregulated | GO: 0016477 | cell migration | 139 | 15462 | 934 | 29 | 3.33E−09 | 2.50E−06 | GPC6, DCLK1, SOS1, SEMA5A, |
| | | | | | | | | | ADRA2A, CD2, HOXA5, NFATC2, MMP1, |
| | | | | | | | | | SATB2, LRRK2, NR4A1, PRKG1, CDH13, |
| | | | | | | | | | DPP4, CCR1, COL1A2, PTPRC, BMP2, |
| | | | | | | | | | RGCC, TNFRSF11A, ITGB3, WNT4, |
| | | | | | | | | | CXCL5, IL6, TBX5, SLC7A5, CXCL9, ADIPOQ |
| upregulated | GO: 0006928 | cellular | 139 | 15462 | 1413 | 35 | 2.10E−08 | 1.26E−05 | GPC6, DCLK1, ADRA2A, CD2, HOXA5, |
| | | component | | | | | | | NFATC2, NKD1, GFRA1, DPP4, COL1A2, |
| | | movement | | | | | | | ITGB3, RGCC, CXCL9, CNTN1, NR4A3, |
| | | | | | | | | | FPR3, SEMA5A, SOS1, SATB2, MMP1, |
| | | | | | | | | | LRRK2, NR4A1, PRKG1, DLX5, CDH13, |
| | | | | | | | | | PTPRC, BMP2, CCR1, TNFRSF11A, |
| | | | | | | | | | CXCL5, WNT4, IL6, TBX5, SLC7A5, ADIPOQ |
| upregulated | GO: 0048584 | positive | 139 | 15462 | 1359 | 34 | 2.76E−08 | 1.38E−05 | IRF4, SHOX2, FABP4, ADRA2A, |
| | | regulation of | | | | | | | NFATC2, NPAS2, NKD1, GDF6, FYB, |
| | | response to | | | | | | | ITGB3, RGCC, LCP2, CXCL9, TSPAN5, |
| | | stimulus | | | | | | | TLR1, SEMA5A, SOS1, PLA2R1, MDFIC, |
| | | | | | | | | | ALOX5AP, CD36, LRRK2, STAP1, BIRC3, |
| | | | | | | | | | DLX5, CDH13, PTPRC, BMP2, CCR1, |
| | | | | | | | | | TNFRSF11A, WNT4, IL6, SKAP2, ADIPOQ |
| upregulated | GO: 0032501 | multicellular | 139 | 15462 | 6071 | 84 | 3.32E−07 | 0.000138277 | SHOX2, IRF4, SPATA9, FABP4, TNNI2, |
| | | organismal | | | | | | | NPAS2, NPTX1, COL1A2, LCP2, ASIC2, |
| | | process | | | | | | | BCHE, FOXO1, CNTN1, PAPPA, TLR1, |
| | | | | | | | | | PRELP, TENM3, SOS1, CHRNA1, |
| | | | | | | | | | PLA2R1, FEM1B, ALOX5AP, FOSL2, |
| | | | | | | | | | PIEZO2, CD36, MMP1, SATB2, PRKG1, |
| | | | | | | | | | PDE1A, GUCY1A3, TNFRSF11A, IL6, |
| | | | | | | | | | TBX5, SLC7A5, DCLK1, ADRA2A, CD2, |
| | | | | | | | | | COL12A1, THSD7A, HOXA5, IGJ, |
| | | | | | | | | | NFATC2, PDLIM5, LSAMP, NKD1, GDF6, |
| | | | | | | | | | CELF2, GFRA1, IL18R1, JAKMIP1, DPP4, |
| | | | | | | | | | HIVEP3, CAMK2D, ITGB3, RGCC, |
| | | | | | | | | | PRDM6, BNC2, ERCC1, TFPI2, NR4A3, |
| | | | | | | | | | HMCN1, SEMA5A, EFEMP1, LMOD1, |
| | | | | | | | | | CALB1, DOCK8, CRISPLD2, FBXO32, |
| | | | | | | | | | ELN, LRRK2, BIRC3, NR4A1, DLX5, |
| | | | | | | | | | MEGF10, SDC2, GCNT4, CDH13, |
| | | | | | | | | | ANGPTL4, PTPRC, CCR1, BMP2, WNT4, NAMPT, ADIPOQ |
| upregulated | GO: 0044707 | single- | 139 | 15462 | 5870 | 82 | 3.69E−07 | 0.000138277 | SHOX2, IRF4, SPATA9, FABP4, TNNI2, |
| | | multicellular | | | | | | | NPAS2, NPTX1, COL1A2, LCP2, ASIC2, |
| | | organism | | | | | | | BCHE, FOXO1, CNTN1, TLR1, PRELP, |
| | | process | | | | | | | TENM3, SOS1, CHRNA1, PLA2R1, |
| | | | | | | | | | FEM1B, ALOX5AP, PIEZO2, CD36, |
| | | | | | | | | | MMP1, SATB2, PRKG1, PDE1A, |
| | | | | | | | | | GUCY1A3, TNFRSF11A, IL6, TBX5, |
| | | | | | | | | | SLC7A5, DCLK1, ADRA2A, CD2, |
| | | | | | | | | | COL12A1, THSD7A, HOXA5, IGJ, |
| | | | | | | | | | NFATC2, PDLIM5, LSAMP, NKD1, GDF6, |
| | | | | | | | | | CELF2, GFRA1, IL18R1, JAKMIP1, DPP4, |
| | | | | | | | | | CAMK2D, HIVEP3, ITGB3, RGCC, |
| | | | | | | | | | PRDM6, BNC2, ERCC1, TFPI2, NR4A3, |
| | | | | | | | | | HMCN1, SEMA5A, EFEMP1, LMOD1, |
| | | | | | | | | | CALB1, DOCK8, CRISPLD2, FBXO32, |
| | | | | | | | | | ELN, LRRK2, BIRC3, NR4A1, DLX5, |
| | | | | | | | | | MEGF10, SDC2, GCNT4, CDH13, |
| | | | | | | | | | ANGPTL4, PTPRC, CCR1, BMP2, WNT4, NAMPT, ADIPOQ |
| upregulated | GO: 0009611 | response to | 139 | 15462 | 1138 | 28 | 8.51E−07 | 0.000283846 | TLR1, SOS1, FABP4, ADRA2A, CD2, |
| | | wounding | | | | | | | ALOX5AP, CD36, MMP1, DOCK8, BIRC3, |
| | | | | | | | | | PRKG1, PDE1A, SDC2, AOX1, CCR1, |
| | | | | | | | | | BMP2, COL1A2, GUCY1A3, TNFRSF11A, |
| | | | | | | | | | ITGB3, WNT4, LCP2, IL6, ASIC2, |
| | | | | | | | | | SLC7A5, TFPI2, CXCL9, ADIPOQ |
| upregulated | GO: 0050896 | response to | 139 | 15462 | 7230 | 93 | 1.19E−06 | 0.000357699 | TAGAP, FAM129A, P2RY8, SHOX2, |
| | | stimulus | | | | | | | IRF4, FABP4, RAB30, BNIP3L, TFEC, |
| | | | | | | | | | NPAS2, GRP, NPTX1, AOX1, COL1A2, |
| | | | | | | | | | LCP2, ASIC2, BCHE, FOXO1, |
| | | | | | | | | | ARHGAP42, CNTN1, PAPPA, TSPAN5, |
| | | | | | | | | | TLR1, TENM3, SOS1, CHRNA1, PLA2R1, |
| | | | | | | | | | MDFIC, FEM1B, ALOX5AP, FOSL2, |
| | | | | | | | | | CD36, PIEZO2, MMP1, SATB2, GPR34, |
| | | | | | | | | | PRKG1, PDE1A, GUCY1A3, TNFRSF11A, |
| | | | | | | | | | IL6, SLC7A5, GPC6, CBLC, DCLK1, |
| | | | | | | | | | ADRA2A, CD2, NFATC2, IGJ, NKD1, |
| | | | | | | | | | GDF6, GFRA1, RASGRF2, IL18R1, |
| | | | | | | | | | DEPDC7, DPP4, FYB, CAMK2D, ITGB3, |
| | | | | | | | | | RGCC, ERCC1, TFPI2, ADH1B, CXCL9, |
| | | | | | | | | | SLC22A3, NR4A3, SLAMF7, HMCN1, |
| | | | | | | | | | FPR3, SEMA5A, CLEC1A, EFEMP1, |
| | | | | | | | | | MCTP1, CALB1, DOCK8, LRRK2, |
| | | | | | | | | | FBXO32, STAP1, BIRC3, NR4A1, DLX5, |
| | | | | | | | | | SDC2, GCNT4, CDH13, ANGPTL4, |
| | | | | | | | | | CCR1, BMP2, PTPRC, WNT4, CXCL5, NAMPT, SKAP2, ADIPOQ |
| upregulated | GO: 0048731 | system | 139 | 15462 | 3615 | 57 | 2.69E−06 | 0.000685492 | IRF4, DCLK1, SHOX2, CD2, COL12A1, |
| | | development | | | | | | | THSD7A, HOXA5, PDLIM5, LSAMP, |
| | | | | | | | | | NPAS2, NKD1, GDF6, NPTX1, GFRA1, |
| | | | | | | | | | IL18R1, COL1A2, HIVEP3, ITGB3, RGCC, |
| | | | | | | | | | PRDM6, BNC2, ERCC1, ASIC2, BCHE, |
| | | | | | | | | | CNTN1, FOXO1, NR4A3, PRELP, |
| | | | | | | | | | CHRNA1, TENM3, SEMA5A, SOS1, |
| | | | | | | | | | EFEMP1, FEM1B, CALB1, SATB2, |
| | | | | | | | | | CRISPLD2, ELN, LRRK2, NR4A1, |
| | | | | | | | | | PRKG1, DLX5, MEGF10, SDC2, GCNT4, |
| | | | | | | | | | CDH13, ANGPTL4, PTPRC, BMP2, |
| | | | | | | | | | CCR1, TNFRSF11A, WNT4, IL6, TBX5, NAMPT, SLC7A5, ADIPOQ |
| upregulated | GO: 0006950 | response to | 139 | 15462 | 3072 | 51 | 2.80E−06 | 0.000685492 | FAM129A, IRF4, FABP4, ADRA2A, CD2, |
| | | stress | | | | | | | IGJ, NFATC2, BNIP3L, TFEC, NPAS2, |
| | | | | | | | | | NKD1, DPP4, AOX1, COL1A2, CAMK2D, |
| | | | | | | | | | ITGB3, RGCC, LCP2, ERCC1, ASIC2, |
| | | | | | | | | | TFPI2, CXCL9, FOXO1, NR4A3, SLAMF7, |
| | | | | | | | | | TLR1, SOS1, CLEC1A, MDFIC, PLA2R1, |
| | | | | | | | | | FEM1B, ALOX5AP, CD36, MMP1, |
| | | | | | | | | | DOCK8, LRRK2, BIRC3, NR4A1, PRKG1, |
| | | | | | | | | | SDC2, PDE1A, ANGPTL4, PTPRC, |
| | | | | | | | | | GUCY1A3, BMP2, CCR1, TNFRSF11A, |
| | | | | | | | | | WNT4, IL6, SLC7A5, ADIPOQ |
| upregulated | GO: 0030154 | cell | 139 | 15462 | 2989 | 50 | 2.97E−06 | 0.000685492 | IRF4, DCLK1, SHOX2, FABP4, SPATA9, |
| | | differentiation | | | | | | | CD2, THSD7A, HOXA5, PDLIM5, NKD1, |
| | | | | | | | | | GDF6, NPTX1, GFRA1, IL18R1, HIVEP3, |
| | | | | | | | | | ITGB3, RGCC, PRDM6, ERCC1, BCHE, |
| | | | | | | | | | FOXO1, CNTN1, PAPPA, NR4A3, |
| | | | | | | | | | STEAP4, TENM3, SOS1, SEMA5A, |
| | | | | | | | | | RUNX1T1, EFEMP1, FEM1B, CD36, |
| | | | | | | | | | SATB2, LRRK2, NR4A1, PRKG1, DLX5, |
| | | | | | | | | | MEGF10, SDC2, ANGPTL4, PTPRC, |
| | | | | | | | | | BMP2, CCR1, TNFRSF11A, FBXO40, |
| | | | | | | | | | WNT4, IL6, TBX5, SLC7A5, ADIPOQ |
| upregulated | GO: 0009967 | positive | 139 | 15462 | 946 | 24 | 3.50E−06 | 0.000750038 | TSPAN5, SOS1, SEMA5A, SHOX2, |
| | | regulation of | | | | | | | ADRA2A, MDFIC, PLA2R1, CD36, |
| | | signal | | | | | | | LRRK2, NKD1, STAP1, GDF6, DLX5, |
| | | transduction | | | | | | | CDH13, CCR1, PTPRC, BMP2, |
| | | | | | | | | | TNFRSF11A, ITGB3, WNT4, IL6, CXCL9, SKAP2, ADIPOQ |
| upregulated | GO: 0009605 | response to | 139 | 15462 | 1775 | 35 | 4.95E−06 | 0.000943396 | GPC6, DCLK1, FABP4, IGJ, BNIP3L, |
| | | external stimulus | | | | | | | GFRA1, ITGB3, ERCC1, ASIC2, CXCL9, |
| | | | | | | | | | SLC22A3, CNTN1, FOXO1, NR4A3, |
| | | | | | | | | | FPR3, TLR1, SEMA5A, SOS1, ALOX5AP, |
| | | | | | | | | | FOSL2, PIEZO2, CD36, BIRC3, NR4A1, |
| | | | | | | | | | DLX5, SDC2, CDH13, CCR1, GUCY1A3, |
| | | | | | | | | | PTPRC, TNFRSF11A, CXCL5, WNT4, IL6, ADIPOQ |
| upregulated | GO: 0048518 | positive | 139 | 15462 | 3873 | 59 | 5.03E−06 | 0.000943396 | FAM129A, IRF4, SHOX2, FABP4, |
| | | regulation of | | | | | | | ADRA2A, CD2, TNNI2, HOXA5, IGJ, |
| | | biological | | | | | | | NFATC2, BNIP3L, NPAS2, NKD1, GDF6, |
| | | process | | | | | | | IL18R1, RASGRF2, DPP4, PNMA2, |
| | | | | | | | | | HIVEP3, FYB, CAMK2D, ITGB3, RGCC, |
| | | | | | | | | | LCP2, ASIC2, CXCL9, CNTN1, FOXO1, |
| | | | | | | | | | NR4A3, TSPAN5, TLR1, TENM3, |
| | | | | | | | | | SEMA5A, SOS1, PLA2R1, MDFIC, |
| | | | | | | | | | ALOX5AP, FOSL2, CD36, SATB2, |
| | | | | | | | | | LRRK2, STAP1, BIRC3, NR4A1, DLX5, |
| | | | | | | | | | CDH13, ANGPTL4, PTPRC, GUCY1A3, |
| | | | | | | | | | BMP2, CCR1, TNFRSF11A, CXCL5, |
| | | | | | | | | | WNT4, IL6, TBX5, NAMPT, SKAP2, ADIPOQ |
| upregulated | GO: 0044699 | single-organism | 139 | 15462 | 12224 | 129 | 7.63E−06 | 0.00132686 | TAGAP, P2RY8, SHOX2, IRF4, SPATA9, |
| | | process | | | | | | | FABP4, HS3ST3B1, TNNI2, RAB30, |
| | | | | | | | | | BNIP3L, NPAS2, MFAP5, GRP, NPTX1, |
| | | | | | | | | | PNMA2, AOX1, COL1A2, LCP2, ASIC2, |
| | | | | | | | | | BCHE, FOXO1, ARHGAP42, CNTN1, |
| | | | | | | | | | PAPPA, TSPAN5, CRLS1, TLR1, PRELP, |
| | | | | | | | | | STEAP4, TENM3, CHRNA1, SOS1, |
| | | | | | | | | | MDFIC, PLA2R1, FEM1B, ALOX5AP, |
| | | | | | | | | | FOSL2, PIEZO2, CD36, MMP1, SATB2, |
| | | | | | | | | | GPR34, PRKG1, PDE1A, GUCY1A3, |
| | | | | | | | | | TNFRSF11A, GALNT14, CHSY3, IL6, |
| | | | | | | | | | CBLN4, TBX5, SLC7A5, GPC6, CBLC, |
| | | | | | | | | | DCLK1, CD2, ADRA2A, COL12A1, |
| | | | | | | | | | THSD7A, HOXA5, IGJ, NFATC2, PDLIM5, |
| | | | | | | | | | LSAMP, NKD1, GDF6, CELF2, GLRX, |
| | | | | | | | | | GFRA1, RASGRF2, GALNT5, IL18R1, |
| | | | | | | | | | DPP4, DEPDC7, JAKMIP1, FYB, |
| | | | | | | | | | CAMK2D, HIVEP3, RGCC, ITGB3, |
| | | | | | | | | | PRDM6, FM04, BNC2, ERCC1, TFPI2, |
| | | | | | | | | | CXCL9, ADH1B, SLC22A3, NR4A3, |
| | | | | | | | | | HMCN1, FPR3, SLAMF7, SEMA5A, |
| | | | | | | | | | CLEC1A, ADAMTS5, RUNX1T1, |
| | | | | | | | | | EFEMP1, PPP1R3B, LMOD1, MCTP1, |
| | | | | | | | | | DOCK8, CALB1, ATP11A, CRISPLD2, |
| | | | | | | | | | ELN, FBXO32, LRRK2, BIRC3, STAP1, |
| | | | | | | | | | NR4A1, DLX5, CDH13, SDC2, GCNT4, |
| | | | | | | | | | MEGF10, ANGPTL4, PLIN1, BMP2, |
| | | | | | | | | | PTPRC, CCR1, FBXO40, WNT4, CXCL5, |
| | | | | | | | | | SLC24A3, RARRES1, NAMPT, SKAP2, TDO2, ADIPOQ |
| upregulated | GO: 0010647 | positive | 139 | 15462 | 993 | 24 | 7.99E−06 | 0.00132686 | TSPAN5, SOS1, SEMA5A, SHOX2, |
| | | regulation of cell | | | | | | | ADRA2A, MDFIC, PLA2R1, CD36, |
| | | communication | | | | | | | LRRK2, NKD1, STAP1, GDF6, DLX5, |
| | | | | | | | | | CDH13, CCR1, PTPRC, BMP2, |
| | | | | | | | | | TNFRSF11A, ITGB3, WNT4, IL6, CXCL9, SKAP2, ADIPOQ |
| upregulated | GO: 0023056 | positive | 139 | 15462 | 996 | 24 | 8.40E−06 | 0.00132686 | TSPAN5, SOS1, SEMA5A, SHOX2, |
| | | regulation of | | | | | | | ADRA2A, MDFIC, PLA2R1, CD36, |
| | | signaling | | | | | | | LRRK2, NKD1, STAP1, GDF6, DLX5, |
| | | | | | | | | | CDH13, CCR1, PTPRC, BMP2, |
| | | | | | | | | | TNFRSF11A, ITGB3, WNT4, IL6, CXCL9, SKAP2, ADIPOQ |
| upregulated | GO: 0051716 | cellular response | 139 | 15462 | 5866 | 78 | 9.63E−06 | 0.001444542 | TAGAP, FAM129A, P2RY8, SHOX2, |
| | | to stimulus | | | | | | | IRF4, FABP4, RAB30, BNIP3L, TFEC, |
| | | | | | | | | | NPAS2, GRP, NPTX1, COL1A2, LCP2, |
| | | | | | | | | | ASIC2, FOXO1, ARHGAP42, CNTN1, |
| | | | | | | | | | TSPAN5, TLR1, TENM3, SOS1, CHRNA1, |
| | | | | | | | | | PLA2R1, MDFIC, FEM1B, ALOX5AP, |
| | | | | | | | | | FOSL2, CD36, SATB2, GPR34, PRKG1, |
| | | | | | | | | | PDE1A, GUCY1A3, TNFRSF11A, IL6, |
| | | | | | | | | | GPC6, CBLC, DCLK1, ADRA2A, CD2, |
| | | | | | | | | | NFATC2, NKD1, GDF6, GFRA1, IL18R1, |
| | | | | | | | | | RASGRF2, DEPDC7, FYB, CAMK2D, |
| | | | | | | | | | ITGB3, RGCC, ERCC1, ADH1B, CXCL9, |
| | | | | | | | | | NR4A3, FPR3, SEMA5A, CLEC1A, |
| | | | | | | | | | EFEMP1, MCTP1, CALB1, DOCK8, |
| | | | | | | | | | LRRK2, STAP1, BIRC3, NR4A1, DLX5, |
| | | | | | | | | | SDC2, CDH13, CCR1, BMP2, PTPRC, |
| | | | | | | | | | CXCL5, WNT4, NAMPT, SKAP2, ADIPOQ |
| upregulated | GO: 0048869 | cellular | 139 | 15462 | 3117 | 50 | 1.01E−05 | 0.001450236 | IRF4, DCLK1, SHOX2, FABP4, SPATA9, |
| | | developmental | | | | | | | CD2, THSD7A, HOXA5, PDLIM5, NKD1, |
| | | process | | | | | | | GDF6, NPTX1, GFRA1, IL18R1, HIVEP3, |
| | | | | | | | | | ITGB3, RGCC, PRDM6, ERCC1, BCHE, |
| | | | | | | | | | FOXO1, CNTN1, PAPPA, NR4A3, |
| | | | | | | | | | STEAP4, TENM3, SOS1, SEMA5A, |
| | | | | | | | | | RUNX1T1, EFEMP1, FEM1B, CD36, |
| | | | | | | | | | SATB2, LRRK2, NR4A1, PRKG1, DLX5, |
| | | | | | | | | | MEGF10, SDC2, ANGPTL4, PTPRC, |
| | | | | | | | | | BMP2, CCR1, TNFRSF11A, FBXO40, |
| | | | | | | | | | WNT4, IL6, TBX5, SLC7A5, ADIPOQ |
| upregulated | GO: 0007596 | blood | 139 | 15462 | 500 | 16 | 1.09E−05 | 0.001468108 | SOS1, ADRA2A, CD2, CD36, MMP1, |
| | | coagulation | | | | | | | DOCK8, PRKG1, PDE1A, COL1A2, |
| | | | | | | | | | GUCY1A3, ITGB3, LCP2, IL6, ASIC2, TFPI2, SLC7A5 |
| upregulated | GO: 0042060 | wound healing | 139 | 15462 | 620 | 18 | 1.13E−05 | 0.001468108 | SOS1, ADRA2A, CD2, CD36, MMP1, |
| | | | | | | | | | DOCK8, PRKG1, PDE1A, SDC2, |
| | | | | | | | | | COL1A2, GUCY1A3, ITGB3, LCP2, |
| | | | | | | | | | WNT4, IL6, ASIC2, SLC7A5, TFPI2 |
| upregulated | GO: 0050817 | coagulation | 139 | 15462 | 503 | 16 | 1.18E−05 | 0.001470427 | SOS1, ADRA2A, CD2, CD36, MMP1, |
| | | | | | | | | | DOCK8, PRKG1, PDE1A, COL1A2, |
| | | | | | | | | | GUCY1A3, ITGB3, LCP2, IL6, ASIC2, TFPI2, SLC7A5 |
| upregulated | GO: 0007599 | hemostasis | 139 | 15462 | 505 | 16 | 1.24E−05 | 0.001482804 | SOS1, ADRA2A, CD2, CD36, MMP1, |
| | | | | | | | | | DOCK8, PRKG1, PDE1A, COL1A2, |
| | | | | | | | | | GUCY1A3, ITGB3, LCP2, IL6, ASIC2, TFPI2, SLC7A5 |
| upregulated | GO: 0051239 | regulation of | 139 | 15462 | 1943 | 36 | 1.42E−05 | 0.001641822 | IRF4, SHOX2, ADRA2A, CD2, HOXA5, |
| | | multicellular | | | | | | | PDLIM5, NKD1, CELF2, GDF6, IL18R1, |
| | | organismal | | | | | | | CAMK2D, ITGB3, RGCC, ASIC2, CNTN1, |
| | | process | | | | | | | FOXO1, TLR1, SEMA5A, TENM3, |
| | | | | | | | | | EFEMP1, CD36, LRRK2, FBXO32, |
| | | | | | | | | | BIRC3, DLX5, SDC2, MEGF10, |
| | | | | | | | | | ANGPTL4, GUCY1A3, BMP2, CCR1, |
| | | | | | | | | | TNFRSF11A, WNT4, IL6, TBX5, ADIPOQ |
| upregulated | GO: 0001775 | cell activation | 139 | 15462 | 763 | 20 | 1.58E−05 | 0.001753348 | SLAMF7, TLR1, SOS1, IRF4, ADRA2A, |
| | | | | | | | | | CD2, NFATC2, CD36, DOCK8, IL18R1, |
| | | | | | | | | | DPP4, COL1A2, PTPRC, RGCC, ITGB3, |
| | | | | | | | | | WNT4, LCP2, IL6, ERCC1, SKAP2 |
| upregulated | GO: 0043068 | positive | 139 | 15462 | 352 | 13 | 1.74E−05 | 0.001865462 | NR4A3, LRRK2, NR4A1, RASGRF2, |
| | | regulation of | | | | | | | PNMA2, SOS1, BMP2, RGCC, IL6, |
| | | programmed cell | | | | | | | HOXA5, BNIP3L, ADIPOQ, FOXO1 |
| | | death | | | | | | | |
| upregulated | GO: 0042127 | regulation of cell | 139 | 15462 | 1263 | 27 | 1.88E−05 | 0.001869591 | SHOX2, SEMA5A, ADRA2A, HOXA5, |
| | | proliferation | | | | | | | NFATC2, FOSL2, LRRK2, NR4A1, DLX5, |
| | | | | | | | | | PDE1A, CDH13, DPP4, BMP2, PTPRC, |
| | | | | | | | | | TNFRSF11A, ITGB3, RGCC, CXCL5, IL6, |
| | | | | | | | | | RARRES1, TBX5, NAMPT, CXCL9, |
| | | | | | | | | | BCHE, SKAP2, FOXO1, ADIPOQ |
| upregulated | GO: 0044700 | single organism | 139 | 15462 | 5438 | 73 | 1.93E−05 | 0.001869591 | TAGAP, P2RY8, SHOX2, IRF4, RAB30, |
| | | signaling | | | | | | | BNIP3L, NPAS2, GRP, NPTX1, COL1A2, |
| | | | | | | | | | LCP2, ASIC2, BCHE, FOXO1, |
| | | | | | | | | | ARHGAP42, CNTN1, TSPAN5, TLR1, |
| | | | | | | | | | TENM3, SOS1, CHRNA1, PLA2R1, |
| | | | | | | | | | MDFIC, FEM1B, CD36, GPR34, PRKG1, |
| | | | | | | | | | PDE1A, GUCY1A3, TNFRSF11A, IL6, |
| | | | | | | | | | TBX5, GPC6, CBLC, DCLK1, ADRA2A, |
| | | | | | | | | | CD2, HOXA5, NFATC2, PDLIM5, NKD1, |
| | | | | | | | | | GDF6, GFRA1, IL18R1, RASGRF2, |
| | | | | | | | | | DEPDC7, FYB, CAMK2D, ITGB3, CXCL9, |
| | | | | | | | | | NR4A3, FPR3, SEMA5A, CLEC1A, |
| | | | | | | | | | EFEMP1, MCTP1, CALB1, DOCK8, |
| | | | | | | | | | LRRK2, STAP1, BIRC3, NR4A1, DLX5, |
| | | | | | | | | | SDC2, CDH13, CCR1, BMP2, PTPRC, |
| | | | | | | | | | CXCL5, WNT4, NAMPT, SKAP2, ADIPOQ |
| upregulated | GO: 0023052 | signaling | 139 | 15462 | 5438 | 73 | 1.93E−05 | 0.001869591 | TAGAP, P2RY8, SHOX2, IRF4, RAB30, |
| | | | | | | | | | BNIP3L, NPAS2, GRP, NPTX1, COL1A2, |
| | | | | | | | | | LCP2, ASIC2, BCHE, FOXO1, |
| | | | | | | | | | ARHGAP42, CNTN1, TSPAN5, TLR1, |
| | | | | | | | | | TENM3, SOS1, CHRNA1, PLA2R1, |
| | | | | | | | | | MDFIC, FEM1B, CD36, GPR34, PRKG1, |
| | | | | | | | | | PDE1A, GUCY1A3, TNFRSF11A, IL6, |
| | | | | | | | | | TBX5, GPC6, CBLC, DCLK1, ADRA2A, |
| | | | | | | | | | CD2, HOXA5, NFATC2, PDLIM5, NKD1, |
| | | | | | | | | | GDF6, GFRA1, IL18R1, RASGRF2, |
| | | | | | | | | | DEPDC7, FYB, CAMK2D, ITGB3, CXCL9, |
| | | | | | | | | | NR4A3, FPR3, SEMA5A, CLEC1A, |
| | | | | | | | | | EFEMP1, MCTP1, CALB1, DOCK8, |
| | | | | | | | | | LRRK2, STAP1, BIRC3, NR4A1, DLX5, |
| | | | | | | | | | SDC2, CDH13, CCR1, BMP2, PTPRC, |
| | | | | | | | | | CXCL5, WNT4, NAMPT, SKAP2, ADIPOQ |
| upregulated | GO: 0030198 | extracellular | 139 | 15462 | 361 | 13 | 2.27E−05 | 0.002063385 | CRISPLD2, MFAP5, ELN, SDC2, DPP4, |
| | | matrix | | | | | | | BMP2, ADAMTS5, COL1A2, RGCC, |
| | | organization | | | | | | | EFEMP1, ITGB3, COL12A1, MMP1 |
| upregulated | GO: 0043062 | extracellular | 139 | 15462 | 361 | 13 | 2.27E−05 | 0.002063385 | CRISPLD2, MFAP5, ELN, SDC2, DPP4, |
| | | structure | | | | | | | BMP2, ADAMTS5, COL1A2, RGCC, |
| | | organization | | | | | | | EFEMP1, ITGB3, COL12A1, MMP1 |
| upregulated | GO: 0007154 | cell | 139 | 15462 | 5493 | 73 | 2.85E−05 | 0.002514438 | TAGAP, P2RY8, SHOX2, IRF4, RAB30, |
| | | communication | | | | | | | BNIP3L, NPAS2, GRP, NPTX1, COL1A2, |
| | | | | | | | | | LCP2, ASIC2, BCHE, FOXO1, |
| | | | | | | | | | ARHGAP42, CNTN1, TSPAN5, TLR1, |
| | | | | | | | | | TENM3, SOS1, CHRNA1, PLA2R1, |
| | | | | | | | | | MDFIC, FEM1B, CD36, GPR34, PRKG1, |
| | | | | | | | | | PDE1A, GUCY1A3, TNFRSF11A, IL6, |
| | | | | | | | | | TBX5, GPC6, CBLC, DCLK1, ADRA2A, |
| | | | | | | | | | CD2, HOXA5, NFATC2, PDLIM5, NKD1, |
| | | | | | | | | | GDF6, GFRA1, IL18R1, RASGRF2, |
| | | | | | | | | | DEPDC7, FYB, CAMK2D, ITGB3, CXCL9, |
| | | | | | | | | | NR4A3, FPR3, SEMA5A, CLEC1A, |
| | | | | | | | | | EFEMP1, MCTP1, CALB1, DOCK8, |
| | | | | | | | | | LRRK2, STAP1, BIRC3, NR4A1, DLX5, |
| | | | | | | | | | SDC2, CDH13, CCR1, BMP2, PTPRC, |
| | | | | | | | | | CXCL5, WNT4, NAMPT, SKAP2, ADIPOQ |
| upregulated | GO: 0010942 | positive | 139 | 15462 | 372 | 13 | 3.10E−05 | 0.002659811 | NR4A3, LRRK2, NR4A1, RASGRF2, |
| | | regulation of cell | | | | | | | PNMA2, SOS1, BMP2, RGCC, IL6, |
| | | death | | | | | | | HOXA5, BNIP3L, ADIPOQ, FOXO1 |
| upregulated | GO: 0045595 | regulation of cell | 139 | 15462 | 1153 | 25 | 3.20E−05 | 0.002668879 | IRF4, SHOX2, TENM3, SEMA5A, |
| | | differentiation | | | | | | | EFEMP1, CD2, HOXA5, PDLIM5, CD36, |
| | | | | | | | | | LRRK2, GDF6, DLX5, MEGF10, SDC2, |
| | | | | | | | | | CCR1, BMP2, ITGB3, RGCC, WNT4, |
| | | | | | | | | | PRDM6, IL6, TBX5, FOXO1, ADIPOQ, CNTN1 |
| upregulated | GO: 0048583 | regulation of | 139 | 15462 | 2724 | 44 | 3.85E−05 | 0.003122791 | TAGAP, CBLC, IRF4, SHOX2, FABP4, |
| | | response to | | | | | | | ADRA2A, NFATC2, NPAS2, NKD1, GDF6, |
| | | stimulus | | | | | | | IL18R1, RASGRF2, DEPDC7, FYB, |
| | | | | | | | | | CAMK2D, ITGB3, RGCC, LCP2, ASIC2, |
| | | | | | | | | | CXCL9, FOXO1, TSPAN5, TLR1, SOS1, |
| | | | | | | | | | SEMA5A, MDFIC, PLA2R1, FEM1B, |
| | | | | | | | | | ALOX5AP, CD36, FBXO32, LRRK2, |
| | | | | | | | | | STAP1, BIRC3, DLX5, CDH13, PTPRC, |
| | | | | | | | | | BMP2, CCR1, TNFRSF11A, WNT4, IL6, SKAP2, ADIPOQ |
| upregulated | GO: 0007165 | signal | 139 | 15462 | 4935 | 67 | 4.33E−05 | 0.003415746 | TAGAP, P2RY8, SHOX2, IRF4, RAB30, |
| | | transduction | | | | | | | BNIP3L, NPAS2, GRP, COL1A2, LCP2, |
| | | | | | | | | | ASIC2, FOXO1, ARHGAP42, CNTN1, |
| | | | | | | | | | TSPAN5, TLR1, TENM3, SOS1, CHRNA1, |
| | | | | | | | | | PLA2R1, MDFIC, FEM1B, CD36, GPR34, |
| | | | | | | | | | PRKG1, PDE1A, GUCY1A3, TNFRSF11A, |
| | | | | | | | | | IL6, GPC6, CBLC, DCLK1, CD2, |
| | | | | | | | | | ADRA2A, NFATC2, NKD1, GDF6, |
| | | | | | | | | | GFRA1, IL18R1, RASGRF2, DEPDC7, |
| | | | | | | | | | FYB, CAMK2D, ITGB3, CXCL9, NR4A3, |
| | | | | | | | | | FPR3, SEMA5A, CLEC1A, EFEMP1, |
| | | | | | | | | | MCTP1, DOCK8, LRRK2, STAP1, BIRC3, |
| | | | | | | | | | NR4A1, DLX5, SDC2, CDH13, PTPRC, |
| | | | | | | | | | CCR1, BMP2, CXCL5, WNT4, NAMPT, SKAP2, ADIPOQ |
| upregulated | GO: 0044767 | single-organism | 139 | 15462 | 4741 | 65 | 4.59E−05 | 0.003529038 | SHOX2, IRF4, SPATA9, FABP4, NPAS2, |
| | | developmental | | | | | | | NPTX1, COL1A2, ASIC2, BCHE, FOXO1, |
| | | process | | | | | | | CNTN1, PAPPA, STEAP4, PRELP, |
| | | | | | | | | | TENM3, SOS1, CHRNA1, PLA2R1, |
| | | | | | | | | | FEM1B, CD36, SATB2, PRKG1, |
| | | | | | | | | | TNFRSF11A, IL6, TBX5, SLC7A5, |
| | | | | | | | | | DCLK1, CD2, COL12A1, THSD7A, |
| | | | | | | | | | HOXA5, PDLIM5, LSAMP, NKD1, GDF6, |
| | | | | | | | | | GFRA1, IL18R1, HIVEP3, RGCC, ITGB3, |
| | | | | | | | | | PRDM6, BNC2, ERCC1, NR4A3, |
| | | | | | | | | | SEMA5A, RUNX1T1, EFEMP1, CALB1, |
| | | | | | | | | | ELN, LRRK2, CRISPLD2, NR4A1, DLX5, |
| | | | | | | | | | SDC2, GCNT4, CDH13, MEGF10, |
| | | | | | | | | | ANGPTL4, PTPRC, BMP2, CCR1, |
| | | | | | | | | | FBXO40, WNT4, NAMPT, ADIPOQ |
| upregulated | GO: 0065008 | regulation of | 139 | 15462 | 2755 | 44 | 5.09E−05 | 0.003822176 | FABP4, ADRA2A, CD2, HOXA5, IGJ, |
| | | biological quality | | | | | | | BNIP3L, PDLIM5, GLRX, NPTX1, |
| | | | | | | | | | COL1A2, CAMK2D, ITGB3, LCP2, |
| | | | | | | | | | ERCC1, ASIC2, TFPI2, CXCL9, SLC22A3, |
| | | | | | | | | | FOXO1, STEAP4, CHRNA1, SOS1, |
| | | | | | | | | | SEMA5A, CD36, PIEZO2, MMP1, DOCK8, |
| | | | | | | | | | CALB1, ATP11A, ELN, LRRK2, PRKG1, |
| | | | | | | | | | GCNT4, PDE1A, ANGPTL4, PTPRC, |
| | | | | | | | | | BMP2, CCR1, GUCY1A3, TNFRSF11A, |
| | | | | | | | | | WNT4, IL6, SLC7A5, ADIPOQ |
| upregulated | GO: 0008283 | cell proliferation | 139 | 15462 | 1668 | 31 | 6.25E−05 | 0.004541682 | SHOX2, SEMA5A, ADRA2A, HOXA5, |
| | | | | | | | | | NFATC2, FOSL2, DOCK8, LRRK2, ELN, |
| | | | | | | | | | NR4A1, DLX5, CDH13, PDE1A, DPP4, |
| | | | | | | | | | BMP2, PTPRC, TNFRSF11A, ITGB3, |
| | | | | | | | | | RGCC, WNT4, CXCL5, IL6, RARRES1, |
| | | | | | | | | | ERCC1, TBX5, NAMPT, CXCL9, BCHE, SKAP2, FOXO1, ADIPOQ |
| upregulated | GO: 0050878 | regulation of | 139 | 15462 | 580 | 16 | 6.59E−05 | 0.004541682 | SOS1, CD2, ADRA2A, CD36, MMP1, |
| | | body fluid levels | | | | | | | DOCK8, PRKG1, PDE1A, COL1A2, |
| | | | | | | | | | GUCY1A3, ITGB3, LCP2, IL6, ASIC2, TFPI2, SLC7A5 |
| upregulated | GO: 0032502 | developmental | 139 | 15462 | 4794 | 65 | 6.67E−05 | 0.004541682 | SHOX2, IRF4, SPATA9, FABP4, NPAS2, |
| | | process | | | | | | | NPTX1, COL1A2, ASIC2, BCHE, FOXO1, |
| | | | | | | | | | CNTN1, PAPPA, STEAP4, PRELP, |
| | | | | | | | | | TENM3, SOS1, CHRNA1, PLA2R1, |
| | | | | | | | | | FEM1B, CD36, SATB2, PRKG1, |
| | | | | | | | | | TNFRSF11A, IL6, TBX5, SLC7A5, |
| | | | | | | | | | DCLK1, CD2, COL12A1, THSD7A, |
| | | | | | | | | | HOXA5, PDLIM5, LSAMP, NKD1, GDF6, |
| | | | | | | | | | GFRA1, IL18R1, HIVEP3, RGCC, ITGB3, |
| | | | | | | | | | PRDM6, BNC2, ERCC1, NR4A3, |
| | | | | | | | | | SEMA5A, RUNX1T1, EFEMP1, CALB1, |
| | | | | | | | | | ELN, LRRK2, CRISPLD2, NR4A1, DLX5, |
| | | | | | | | | | SDC2, GCNT4, CDH13, MEGF10, |
| | | | | | | | | | ANGPTL4, PTPRC, BMP2, CCR1, |
| | | | | | | | | | FBXO40, WNT4, NAMPT, ADIPOQ |
| upregulated | GO: 0065007 | biological | 139 | 15462 | 974 | 109 | 6.70E−05 | 0.004541682 | TAGAP, FAM129A, P2RY8, SHOX2, |
| | | regulation | | | | | | | IRF4, FABP4, TNNI2, RAB30, BNIP3L, |
| | | | | | | | | | TFEC, NPAS2, GRP, NPTX1, PNMA2, |
| | | | | | | | | | COL1A2, LCP2, ASIC2, BCHE, FOXO1, |
| | | | | | | | | | ARHGAP42, CNTN1, TSPAN5, TLR1, |
| | | | | | | | | | STEAP4, TENM3, CHRNA1, SOS1, |
| | | | | | | | | | MDFIC, PLA2R1, FEM1B, ALOX5AP, |
| | | | | | | | | | FOSL2, PIEZO2, CD36, MMP1, SATB2, |
| | | | | | | | | | ZFHX4, GPR34, PRKG1, PDE1A, |
| | | | | | | | | | GUCY1A3, TNFRSF11A, IL6, TBX5, |
| | | | | | | | | | SLC7A5, GPC6, CBLC, DCLK1, CD2, |
| | | | | | | | | | ADRA2A, HOXA5, IGJ, NFATC2, PDLIM5, |
| | | | | | | | | | NKD1, GDF6, CELF2, GLRX, GFRA1, |
| | | | | | | | | | RASGRF2, IL18R1, DPP4, DEPDC7, |
| | | | | | | | | | DHX34, CAMK2D, HIVEP3, FYB, RGCC, |
| | | | | | | | | | ITGB3, PRDM6, BNC2, ERCC1, TFPI2, |
| | | | | | | | | | CXCL9, SLC22A3, NR4A3, FPR3, |
| | | | | | | | | | SLAMF7, SEMA5A, CLEC1A, RFX2, |
| | | | | | | | | | RUNX1T1, EFEMP1, PPP1R3B, MCTP1, |
| | | | | | | | | | DOCK8, CALB1, ATP11A, ELN, FBXO32, |
| | | | | | | | | | LRRK2, BIRC3, STAP1, NR4A1, DLX5, |
| | | | | | | | | | CDH13, SDC2, GCNT4, MEGF10, |
| | | | | | | | | | ANGPTL4, BMP2, PTPRC, CCR1, WNT4, |
| | | | | | | | | | CXCL5, RARRES1, NAMPT, SKAP2, ADIPOQ |
| upregulated | GO: 0043065 | positive | 139 | 15462 | 346 | 12 | 6.81E−05 | 0.004541682 | NR4A3, NR4A1, RASGRF2, PNMA2, |
| | | regulation of | | | | | | | SOS1, BMP2, RGCC, IL6, HOXA5, |
| | | apoptotic | | | | | | | BNIP3L, FOXO1, ADIPOQ |
| | | process | | | | | | | |
| upregulated | GO: 0048522 | positive | 139 | 15462 | 3443 | 51 | 7.47E−05 | 0.004871868 | FAM129A, IRF4, SHOX2, ADRA2A, CD2, |
| | | regulation of | | | | | | | TNNI2, HOXA5, NFATC2, BNIP3L, |
| | | cellular process | | | | | | | NPAS2, NKD1, GDF6, RASGRF2, DPP4, |
| | | | | | | | | | PNMA2, HIVEP3, ITGB3, RGCC, ASIC2, |
| | | | | | | | | | CXCL9, CNTN1, FOXO1, NR4A3, |
| | | | | | | | | | TSPAN5, TLR1, TENM3, SEMA5A, SOS1, |
| | | | | | | | | | PLA2R1, MDFIC, FOSL2, CD36, SATB2, |
| | | | | | | | | | LRRK2, STAP1, BIRC3, NR4A1, DLX5, |
| | | | | | | | | | CDH13, PTPRC, GUCY1A3, BMP2, |
| | | | | | | | | | CCR1, TNFRSF11A, CXCL5, WNT4, IL6, |
| | | | | | | | | | TBX5, NAMPT, SKAP2, ADIPOQ |
| upregulated | GO: 0055095 | lipoprotein | 139 | 15462 | 2 | 2 | 8.02E−05 | 0.005016668 | CDH13, CD36 |
| | | particle mediated | | | | | | | |
| | | signaling | | | | | | | |
| upregulated | GO: 0055096 | low-density | 139 | 15462 | 2 | 2 | 8.02E−05 | 0.005016668 | CDH13, CD36 |
| | | lipoprotein | | | | | | | |
| | | particle mediated | | | | | | | |
| | | signaling | | | | | | | |
| upregulated | GO: 0007169 | transmembrane | 139 | 15462 | 663 | 17 | 9.47E−05 | 0.00576639 | CBLC, SOS1, ADRA2A, EFEMP1, STAP1, |
| | | receptor protein | | | | | | | NR4A1, GFRA1, CDH13, PDE1A, |
| | | tyrosine kinase | | | | | | | RASGRF2, BMP2, ITGB3, LCP2, WNT4, |
| | | signaling | | | | | | | NAMPT, FOXO1, ADIPOQ |
| | | pathway | | | | | | | |
| upregulated | GO: 0002376 | immune system | 139 | 15462 | 2043 | 35 | 9.99E−05 | 0.00576639 | IRF4, CD2, HOXA5, IGJ, NFATC2, |
| | | process | | | | | | | BNIP3L, IL18R1, DPP4, COL1A2, FYB, |
| | | | | | | | | | CAMK2D, ITGB3, RGCC, LCP2, ERCC1, |
| | | | | | | | | | CXCL9, FOXO1, SLAMF7, TLR1, SOS1, |
| | | | | | | | | | CD36, DOCK8, MMP1, BIRC3, NR4A1, |
| | | | | | | | | | PDE1A, PTPRC, CCR1, TNFRSF11A, |
| | | | | | | | | | CXCL5, WNT4, IL6, SLC7A5, SKAP2, ADIPOQ |
| upregulated | GO: 0006935 | Chemotaxis | 139 | 15462 | 601 | 16 | 9.99E−05 | 0.00576639 | NR4A3, FPR3, NR4A1, DLX5, GFRA1, |
| | | | | | | | | | CDH13, SOS1, SEMA5A, CCR1, PTPRC, |
| | | | | | | | | | TNFRSF11A, ITGB3, CXCL5, IL6, CXCL9, CNTN1 |
| upregulated | GO: 0042330 | taxis | 139 | 15462 | 601 | 16 | 9.99E−05 | 0.00576639 | NR4A3, FPR3, NR4A1, DLX5, GFRA1, |
| | | | | | | | | | CDH13, SOS1, SEMA5A, CCR1, PTPRC, |
| | | | | | | | | | TNFRSF11A, ITGB3, CXCL5, IL6, CXCL9, CNTN1 |
| upregulated | GO: 0070887 | cellular response | 139 | 15462 | 2050 | 35 | 0.000107044 | 0.006061141 | IRF4, FABP4, ADRA2A, NPTX1, IL18R1, |
| | | to chemical | | | | | | | RASGRF2, CAMK2D, COL1A2, ITGB3, |
| | | stimulus | | | | | | | RGCC, ADH1B, CXCL9, FOXO1, SOS1, |
| | | | | | | | | | SEMA5A, PLA2R1, FOSL2, ALOX5AP, |
| | | | | | | | | | CD36, SATB2, CALB1, LRRK2, NR4A1, |
| | | | | | | | | | DLX5, CDH13, PDE1A, CCR1, BMP2, |
| | | | | | | | | | PTPRC, TNFRSF11A, WNT4, CXCL5, IL6, NAMPT, ADIPOQ |
| upregulated | GO: 0000904 | cell | 139 | 15462 | 741 | 18 | 0.000115442 | 0.006379291 | NR4A3, DCLK1, SOS1, SEMA5A, |
| | | morphogenesis | | | | | | | SHOX2, PDLIM5, LRRK2, GFRA1, DLX5, |
| | | involved in | | | | | | | NPTX1, SDC2, PTPRC, BMP2, RGCC, |
| | | differentiation | | | | | | | ITGB3, WNT4, TBX5, CNTN1 |
| upregulated | GO: 0009653 | anatomical | 139 | 15462 | 2059 | 35 | 0.000116915 | 0.006379291 | DCLK1, SHOX2, THSD7A, HOXA5, |
| | | structure | | | | | | | PDLIM5, NKD1, NPTX1, GFRA1, |
| | | morphogenesis | | | | | | | COL1A2, ITGB3, RGCC, ERCC1, CNTN1, |
| | | | | | | | | | NR4A3, SOS1, SEMA5A, TENM3, |
| | | | | | | | | | FEM1B, SATB2, CALB1, ELN, LRRK2, |
| | | | | | | | | | CRISPLD2, NR4A1, DLX5, CDH13, |
| | | | | | | | | | GCNT4, SDC2, ANGPTL4, BMP2, |
| | | | | | | | | | PTPRC, WNT4, IL6, TBX5, ADIPOQ |
| upregulated | GO: 0007275 | multicellular | 139 | 15462 | 4180 | 58 | 0.000119924 | 0.006426646 | DCLK1, SHOX2, IRF4, SPATA9, CD2, |
| | | organismal | | | | | | | COL12A1, HOXA5, THSD7A, PDLIM5, |
| | | development | | | | | | | LSAMP, NPAS2, NKD1, GDF6, GFRA1, |
| | | | | | | | | | NPTX1, IL18R1, COL1A2, HIVEP3, |
| | | | | | | | | | ITGB3, RGCC, PRDM6, BNC2, ERCC1, |
| | | | | | | | | | ASIC2, BCHE, FOXO1, CNTN1, NR4A3, |
| | | | | | | | | | PRELP, TENM3, SEMA5A, SOS1, |
| | | | | | | | | | CHRNA1, EFEMP1, FEM1B, CALB1, |
| | | | | | | | | | SATB2, CRISPLD2, LRRK2, ELN, NR4A1, |
| | | | | | | | | | PRKG1, DLX5, MEGF10, CDH13, |
| | | | | | | | | | GCNT4, SDC2, ANGPTL4, CCR1, BMP2, |
| | | | | | | | | | PTPRC, TNFRSF11A, WNT4, IL6, TBX5, |
| | | | | | | | | | NAMPT, SLC7A5, ADIPOQ |
| upregulated | GO: 0001936 | regulation of | 139 | 15462 | 86 | 6 | 0.000123459 | 0.006499984 | RGCC, ITGB3, NR4A1, CDH13, SEMA5A, |
| | | endothelial cell | | | | | | | BMP2 |
| | | proliferation | | | | | | | |
| upregulated | GO: 0048856 | anatomical | 139 | 15462 | 4193 | 58 | 0.00013143 | 0.006800369 | DCLK1, SHOX2, IRF4, CD2, COL12A1, |
| | | structure | | | | | | | HOXA5, THSD7A, PDLIM5, LSAMP, |
| | | development | | | | | | | NPAS2, NKD1, GDF6, GFRA1, NPTX1, |
| | | | | | | | | | IL18R1, COL1A2, HIVEP3, ITGB3, RGCC, |
| | | | | | | | | | PRDM6, BNC2, ERCC1, ASIC2, BCHE, |
| | | | | | | | | | FOXO1, CNTN1, NR4A3, PRELP, |
| | | | | | | | | | TENM3, SEMA5A, SOS1, CHRNA1, |
| | | | | | | | | | EFEMP1, FEM1B, CALB1, SATB2, |
| | | | | | | | | | CRISPLD2, LRRK2, ELN, NR4A1, |
| | | | | | | | | | PRKG1, DLX5, MEGF10, CDH13, |
| | | | | | | | | | GCNT4, SDC2, ANGPTL4, CCR1, BMP2, |
| | | | | | | | | | PTPRC, TNFRSF11A, FBXO40, WNT4, |
| | | | | | | | | | IL6, TBX5, NAMPT, SLC7A5, ADIPOQ |
| upregulated | GO: 0006952 | defense | 139 | 15462 | 1342 | 26 | 0.000142131 | 0.007229409 | SLAMF7, TLR1, IRF4, SOS1, FABP4, |
| | | response | | | | | | | CLEC1A, ADRA2A, IGJ, NFATC2, |
| | | | | | | | | | BNIP3L, ALOX5AP, CD36, BIRC3, |
| | | | | | | | | | NR4A1, PDE1A, AOX1, CCR1, CAMK2D, |
| | | | | | | | | | PTPRC, BMP2, TNFRSF11A, LCP2, IL6, |
| | | | | | | | | | CXCL9, ADIPOQ, FOXO1 |
| upregulated | GO: 0061061 | muscle structure | 139 | 15462 | 442 | 13 | 0.000176874 | 0.008757845 | ELN, NKD1, NR4A1, MEGF10, CHRNA1, |
| | | development | | | | | | | SHOX2, BMP2, HIVEP3, FBXO40, WNT4, PRDM6, IL6, TBX5 |
| upregulated | GO: 0050793 | regulation of | 139 | 15462 | 1520 | 28 | 0.000178017 | 0.008757845 | IRF4, SHOX2, TENM3, SEMA5A, |
| | | developmental | | | | | | | EFEMP1, CD2, HOXA5, PDLIM5, CD36, |
| | | process | | | | | | | LRRK2, NKD1, GDF6, DLX5, MEGF10, |
| | | | | | | | | | SDC2, ANGPTL4, CCR1, BMP2, ITGB3, |
| | | | | | | | | | RGCC, WNT4, PRDM6, IL6, TBX5, |
| | | | | | | | | | ASIC2, CNTN1, FOXO1, ADIPOQ |
| upregulated | GO: 0009893 | positive | 139 | 15462 | 2291 | 37 | 0.000203413 | 0.009750426 | FAM129A, IRF4, SHOX2, ADRA2A, |
| | | regulation of | | | | | | | TNNI2, HOXA5, IGJ, NFATC2, NPAS2, |
| | | metabolic | | | | | | | NKD1, GDF6, HIVEP3, ITGB3, RGCC, |
| | | process | | | | | | | CXCL9, FOXO1, CNTN1, NR4A3, TLR1, |
| | | | | | | | | | MDFIC, CD36, SATB2, LRRK2, BIRC3, |
| | | | | | | | | | NR4A1, DLX5, CDH13, BMP2, CCR1, |
| | | | | | | | | | GUCY1A3, PTPRC, TNFRSF11A, WNT4, |
| | | | | | | | | | IL6, TBX5, NAMPT, ADIPOQ |
| upregulated | GO: 0007167 | enzyme linked | 139 | 15462 | 918 | 20 | 0.000204691 | 0.009750426 | CBLC, SOS1, ADRA2A, EFEMP1, STAP1, |
| | | receptor protein | | | | | | | NR4A1, GDF6, GFRA1, DLX5, CDH13, |
| | | signaling | | | | | | | PDE1A, RASGRF2, COL1A2, BMP2, |
| | | pathway | | | | | | | ITGB3, WNT4, LCP2, NAMPT, FOXO1, ADIPOQ |
| upregulated | GO: 0007166 | cell surface | 139 | 15462 | 3018 | 45 | 0.000214452 | 0.010055783 | P2RY8, CBLC, IRF4, SHOX2, ADRA2A, |
| | | receptor | | | | | | | CD2, NFATC2, NKD1, GDF6, GRP, |
| | | signaling | | | | | | | GFRA1, IL18R1, RASGRF2, COL1A2, |
| | | pathway | | | | | | | FYB, CAMK2D, ITGB3, LCP2, CXCL9, |
| | | | | | | | | | CNTN1, FOXO1, FPR3, TSPAN5, SOS1, |
| | | | | | | | | | CLEC1A, EFEMP1, MDFIC, FEM1B, |
| | | | | | | | | | CD36, LRRK2, STAP1, BIRC3, NR4A1, |
| | | | | | | | | | GPR34, DLX5, CDH13, PDE1A, PTPRC, |
| | | | | | | | | | BMP2, CCR1, TNFRSF11A, WNT4, IL6, NAMPT, ADIPOQ |
| upregulated | GO: 0032844 | regulation of | 139 | 15462 | 281 | 10 | 0.000227344 | 0.010496303 | LRRK2, PTPRC, CAMK2D, ADRA2A, |
| | | homeostatic | | | | | | | ITGB3, TNFRSF11A, HOXA5, ERCC1, CXCL9, FOXO1 |
| | | process | | | | | | | |
| upregulated | GO: 0050679 | positive | 139 | 15462 | 137 | 7 | 0.000237439 | 0.010560772 | ITGB3, IL6, NR4A1, DLX5, CDH13, SEMA5A, BMP2 |
| | | regulation of | | | | | | | |
| | | epithelial cell | | | | | | | |
| | | proliferation | | | | | | | |
| upregulated | GO: 0045715 | negative | 139 | 15462 | 3 | 2 | 0.000239298 | 0.010560772 | ITGB3, ADIPOQ |
| | | regulation of low- | | | | | | | |
| | | density | | | | | | | |
| | | lipoprotein | | | | | | | |
| | | particle receptor | | | | | | | |
| | | biosynthetic | | | | | | | |
| | | process | | | | | | | |
| upregulated | GO: 0071404 | cellular response | 139 | 15462 | 3 | 2 | 0.000239298 | 0.010560772 | CDH13, CD36 |
| | | to low-density | | | | | | | |
| | | lipoprotein | | | | | | | |
| | | particle stimulus | | | | | | | |
| upregulated | GO: 0001938 | positive | 139 | 15462 | 63 | 5 | 0.000252651 | 0.010988488 | ITGB3, NR4A1, CDH13, SEMA5A, BMP2 |
| | | regulation of | | | | | | | |
| | | endothelial cell | | | | | | | |
| | | proliferation | | | | | | | |
| upregulated | GO: 0001816 | cytokine | 139 | 15462 | 522 | 14 | 0.000257435 | 0.011036593 | BIRC3, TLR1, IL18R1, IRF4, FABP4, |
| | | production | | | | | | | RGCC, ADRA2A, CD2, LCP2, IL6, |
| | | | | | | | | | PLA2R1, NFATC2, CD36, ADIPOQ |
| upregulated | GO: 0050678 | regulation of | 139 | 15462 | 234 | 9 | 0.000266093 | 0.011247128 | NR4A1, DLX5, CDH13, SEMA5A, BMP2, |
| | | epithelial cell | | | | | | | ITGB3, RGCC, IL6, HOXA5 |
| | | proliferation | | | | | | | |
| upregulated | GO: 0001935 | endothelial cell | 139 | 15462 | 100 | 6 | 0.000282483 | 0.011672916 | RGCC, ITGB3, NR4A1, CDH13, SEMA5A, |
| | | proliferation | | | | | | | BMP2 |
| upregulated | GO: 0022008 | neurogenesis | 139 | 15462 | 1244 | 24 | 0.000283946 | 0.011672916 | NR4A3, SHOX2, TENM3, DCLK1, SOS1, |
| | | | | | | | | | SEMA5A, PDLIM5, SATB2, LRRK2, |
| | | | | | | | | | NKD1, GDF6, PRKG1, NPTX1, DLX5, |
| | | | | | | | | | GFRA1, SDC2, PTPRC, BMP2, ITGB3, |
| | | | | | | | | | WNT4, PRDM6, IL6, BCHE, CNTN1 |
| upregulated | GO: 0048699 | generation of | 139 | 15462 | 1180 | 23 | 0.000334218 | 0.013553886 | NR4A3, SHOX2, TENM3, DCLK1, SOS1, |
| | | neurons | | | | | | | SEMA5A, PDLIM5, SATB2, LRRK2, |
| | | | | | | | | | NKD1, GDF6, PRKG1, NPTX1, DLX5, |
| | | | | | | | | | GFRA1, SDC2, PTPRC, BMP2, ITGB3, |
| | | | | | | | | | WNT4, IL6, BCHE, CNTN1 |
| upregulated | GO: 0006024 | glycosaminoglycan | 139 | 15462 | 104 | 6 | 0.000349195 | 0.013972438 | GPC6, CHSY3, PRELP, SDC2, GALNT5, |
| | | biosynthetic | | | | | | | HS3ST3B1 |
| | | process | | | | | | | |
| upregulated | GO: 0006023 | aminoglycan | 139 | 15462 | 105 | 6 | 0.000367656 | 0.014517573 | GPC6, CHSY3, PRELP, SDC2, GALNT5, |
| | | biosynthetic | | | | | | | HS3ST3B1 |
| | | process | | | | | | | |
| upregulated | GO: 0040012 | regulation of | 139 | 15462 | 542 | 14 | 0.000376474 | 0.01467272 | LRRK2, NKD1, CDH13, SEMA5A, CCR1, |
| | | locomotion | | | | | | | BMP2, PTPRC, RGCC, ITGB3, ADRA2A, |
| | | | | | | | | | WNT4, IL6, TBX5, ADIPOQ |
| upregulated | GO: 0045444 | fat cell | 139 | 15462 | 149 | 7 | 0.000395959 | 0.015234268 | IL6, STEAP4, RUNX1T1, ADIPOQ, BMP2, |
| | | differentiation | | | | | | | FOXO1, FABP4 |
| upregulated | GO: 0031175 | neuron | 139 | 15462 | 754 | 17 | 0.000431254 | 0.016382191 | NR4A3, TENM3, DCLK1, SOS1, |
| | | projection | | | | | | | SEMA5A, SHOX2, PDLIM5, LRRK2, |
| | | development | | | | | | | PRKG1, GFRA1, DLX5, NPTX1, SDC2, |
| | | | | | | | | | PTPRC, ITGB3, IL6, CNTN1 |
| upregulated | GO: 2000026 | regulation of | 139 | 15462 | 1204 | 23 | 0.000444446 | 0.016672269 | IRF4, SHOX2, TENM3, SEMA5A, |
| | | multicellular | | | | | | | EFEMP1, CD2, HOXA5, PDLIM5, LRRK2, |
| | | organismal | | | | | | | NKD1, GDF6, MEGF10, SDC2, |
| | | development | | | | | | | ANGPTL4, CCR1, BMP2, RGCC, WNT4, |
| | | | | | | | | | IL6, TBX5, ASIC2, ADIPOQ, CNTN1 |
| upregulated | GO: 0071402 | cellular response | 139 | 15462 | 4 | 2 | 0.00047577 | 0.017626986 | CDH13, CD36 |
| | | to lipoprotein | | | | | | | |
| | | particle stimulus | | | | | | | |
| upregulated | GO: 0032963 | collagen | 139 | 15462 | 111 | 6 | 0.000495045 | 0.01811743 | RGCC, WNT4, COL12A1, IL6, COL1A2, MMP1 |
| | | metabolic | | | | | | | |
| | | process | | | | | | | |
| upregulated | GO: 2000145 | regulation of cell | 139 | 15462 | 493 | 13 | 0.000504839 | 0.018253264 | NKD1, CDH13, SEMA5A, CCR1, BMP2, |
| | | motility | | | | | | | PTPRC, RGCC, ADRA2A, ITGB3, WNT4, |
| | | | | | | | | | IL6, TBX5, ADIPOQ |
| upregulated | GO: 0010869 | regulation of | 139 | 15462 | 18 | 3 | 0.000525516 | 0.018457263 | ITGB3, HOXA5, ADIPOQ |
| | | receptor | | | | | | | |
| | | biosynthetic | | | | | | | |
| | | process | | | | | | | |
| upregulated | GO: 0048468 | cell development | 139 | 15462 | 1624 | 28 | 0.000527379 | 0.018457263 | NR4A3, SHOX2, TENM3, DCLK1, SOS1, |
| | | | | | | | | | SEMA5A, HOXA5, FEM1B, PDLIM5, |
| | | | | | | | | | SATB2, LRRK2, GDF6, PRKG1, NPTX1, |
| | | | | | | | | | DLX5, GFRA1, MEGF10, SDC2, BMP2, |
| | | | | | | | | | PTPRC, ITGB3, RGCC, WNT4, IL6, TBX5, |
| | | | | | | | | | ERCC1, CNTN1, ADIPOQ |
| upregulated | GO: 0044763 | single-organism | 139 | 15462 | 10871 | 115 | 0.000534352 | 0.018457263 | TAGAP, P2RY8, SHOX2, IRF4, SPATA9, |
| | | cellular process | | | | | | | FABP4, HS3ST3B1, RAB30, BNIP3L, |
| | | | | | | | | | NPAS2, MFAP5, GRP, NPTX1, PNMA2, |
| | | | | | | | | | AOX1, COL1A2, LCP2, ASIC2, BCHE, |
| | | | | | | | | | FOXO1, ARHGAP42, CNTN1, PAPPA, |
| | | | | | | | | | TSPAN5, CRLS1, TLR1, PRELP, |
| | | | | | | | | | STEAP4, TENM3, CHRNA1, SOS1, |
| | | | | | | | | | MDFIC, PLA2R1, FEM1B, ALOX5AP, |
| | | | | | | | | | FOSL2, PIEZO2, CD36, MMP1, SATB2, |
| | | | | | | | | | GPR34, PRKG1, PDE1A, GUCY1A3, |
| | | | | | | | | | TNFRSF11A, GALNT14, CHSY3, IL6, |
| | | | | | | | | | CBLN4, TBX5, SLC7A5, GPC6, CBLC, |
| | | | | | | | | | DCLK1, CD2, ADRA2A, COL12A1, |
| | | | | | | | | | THSD7A, HOXA5, NFATC2, PDLIM5, |
| | | | | | | | | | NKD1, GDF6, GLRX, GFRA1, RASGRF2, |
| | | | | | | | | | GALNT5, IL18R1, DPP4, DEPDC7, |
| | | | | | | | | | CAMK2D, HIVEP3, FYB, RGCC, ITGB3, |
| | | | | | | | | | PRDM6, ERCC1, CXCL9, SLC22A3, |
| | | | | | | | | | NR4A3, FPR3, SLAMF7, SEMA5A, |
| | | | | | | | | | CLEC1A, ADAMTS5, RUNX1T1, |
| | | | | | | | | | EFEMP1, PPP1R3B, MCTP1, DOCK8, |
| | | | | | | | | | CALB1, CRISPLD2, ELN, LRRK2, BIRC3, |
| | | | | | | | | | STAP1, NR4A1, DLX5, CDH13, GCNT4, |
| | | | | | | | | | SDC2, MEGF10, PLIN1, ANGPTL4, |
| | | | | | | | | | BMP2, PTPRC, CCR1, FBXO40, WNT4, |
| | | | | | | | | | CXCL5, SLC24A3, NAMPT, SKAP2, TDO2, ADIPOQ |
| upregulated | GO: 0022407 | regulation of cell- | 139 | 15462 | 74 | 5 | 0.000535082 | 0.018457263 | RGCC, WNT4, DPP4, ADIPOQ, BMP2 |
| | | cell adhesion | | | | | | | |
| upregulated | GO: 0080134 | regulation of | 139 | 15462 | 842 | 18 | 0.000545998 | 0.018619761 | TLR1, IRF4, FABP4, ADRA2A, PLA2R1, |
| | | response to | | | | | | | MDFIC, FEM1B, ALOX5AP, CD36, |
| | | stress | | | | | | | NPAS2, BIRC3, BMP2, TNFRSF11A, |
| | | | | | | | | | WNT4, IL6, ASIC2, FOXO1, ADIPOQ |
| upregulated | GO: 0048513 | organ | 139 | 15462 | 2597 | 39 | 0.000586126 | 0.01976364 | IRF4, DCLK1, SHOX2, CD2, HOXA5, |
| | | development | | | | | | | NKD1, IL18R1, COL1A2, HIVEP3, RGCC, |
| | | | | | | | | | BNC2, ERCC1, FOXO1, CNTN1, NR4A3, |
| | | | | | | | | | CHRNA1, SEMA5A, TENM3, EFEMP1, |
| | | | | | | | | | FEM1B, SATB2, CALB1, ELN, LRRK2, |
| | | | | | | | | | CRISPLD2, NR4A1, PRKG1, DLX5, |
| | | | | | | | | | MEGF10, GCNT4, CCR1, BMP2, PTPRC, |
| | | | | | | | | | TNFRSF11A, WNT4, IL6, TBX5, NAMPT, ADIPOQ |
| upregulated | GO: 0009888 | tissue | 139 | 15462 | 1474 | 26 | 0.00061424 | 0.02048149 | NR4A3, CHRNA1, SHOX2, SEMA5A, |
| | | development | | | | | | | EFEMP1, HOXA5, FEM1B, CALB1, |
| | | | | | | | | | SATB2, ELN, NKD1, NR4A1, DLX5, |
| | | | | | | | | | MEGF10, GCNT4, CCR1, BMP2, HIVEP3, |
| | | | | | | | | | PTPRC, RGCC, WNT4, IL6, BNC2, TBX5, NAMPT, ADIPOQ |
| upregulated | GO: 0048667 | cell | 139 | 15462 | 572 | 14 | 0.000642114 | 0.020548546 | NR4A3, LRRK2, NPTX1, DLX5, GFRA1, |
| | | morphogenesis | | | | | | | SDC2, DCLK1, SOS1, SEMA5A, SHOX2, |
| | | involved in | | | | | | | PTPRC, ITGB3, PDLIM5, CNTN1 |
| | | neuron | | | | | | | |
| | | differentiation | | | | | | | |
| upregulated | GO: 0022603 | regulation of | 139 | 15462 | 572 | 14 | 0.000642114 | 0.020548546 | LRRK2, NKD1, SDC2, ANGPTL4, |
| | | anatomical | | | | | | | SEMA5A, SHOX2, BMP2, RGCC, WNT4, |
| | | structure | | | | | | | IL6, HOXA5, TBX5, PDLIM5, ADIPOQ |
| | | morphogenesis | | | | | | | |
| upregulated | GO: 1903034 | regulation of | 139 | 15462 | 321 | 10 | 0.000648465 | 0.020548546 | BIRC3, FABP4, TNFRSF11A, ADRA2A, |
| | | response to | | | | | | | WNT4, IL6, ASIC2, ALOX5AP, CD36, |
| | | wounding | | | | | | | ADIPOQ |
| upregulated | GO: 0044259 | multicellular | 139 | 15462 | 117 | 6 | 0.000654448 | 0.020548546 | RGCC, WNT4, COL12A1, IL6, COL1A2, |
| | | organismal | | | | | | | MMP1 |
| | | macromolecule | | | | | | | |
| | | metabolic | | | | | | | |
| | | process | | | | | | | |
| upregulated | GO: 0031325 | positive | 139 | 15462 | 2163 | 34 | 0.000656493 | 0.020548546 | FAM129A, IRF4, SHOX2, ADRA2A, |
| | | regulation of | | | | | | | TNNI2, HOXA5, NFATC2, NPAS2, GDF6, |
| | | cellular | | | | | | | HIVEP3, ITGB3, RGCC, CXCL9, FOXO1, |
| | | metabolic | | | | | | | NR4A3, TLR1, MDFIC, CD36, SATB2, |
| | | process | | | | | | | LRRK2, BIRC3, NR4A1, DLX5, CDH13, |
| | | | | | | | | | CCR1, BMP2, GUCY1A3, PTPRC, |
| | | | | | | | | | TNFRSF11A, WNT4, IL6, TBX5, NAMPT, ADIPOQ |
| upregulated | GO: 0007517 | muscle organ | 139 | 15462 | 265 | 9 | 0.000657334 | 0.020548546 | ELN, NR4A1, MEGF10, CHRNA1, |
| | | development | | | | | | | SHOX2, BMP2, HIVEP3, IL6, TBX5 |
| upregulated | GO: 0030182 | neuron | 139 | 15462 | 1083 | 21 | 0.000667325 | 0.020645792 | NR4A3, SHOX2, TENM3, DCLK1, SOS1, |
| | | differentiation | | | | | | | SEMA5A, PDLIM5, LRRK2, NKD1, GDF6, |
| | | | | | | | | | PRKG1, NPTX1, GFRA1, DLX5, SDC2, |
| | | | | | | | | | PTPRC, BMP2, ITGB3, WNT4, IL6, CNTN1 |
| upregulated | GO: 0048634 | regulation of | 139 | 15462 | 78 | 5 | 0.000681429 | 0.020867036 | IL6, TBX5, MEGF10, SHOX2, BMP2 |
| | | muscle organ | | | | | | | |
| | | development | | | | | | | |
| upregulated | GO: 0060993 | kidney | 139 | 15462 | 45 | 4 | 0.000700174 | 0.021224456 | WNT4, LRRK2, GCNT4, CALB1 |
| | | morphogenesis | | | | | | | |
| upregulated | GO: 0045321 | leukocyte | 139 | 15462 | 578 | 14 | 0.000711007 | 0.021282911 | SLAMF7, TLR1, IL18R1, IRF4, DPP4, |
| | | activation | | | | | | | PTPRC, CD2, WNT4, LCP2, IL6, ERCC1, |
| | | | | | | | | | NFATC2, SKAP2, DOCK8 |
| upregulated | GO: 0008284 | positive | 139 | 15462 | 717 | 16 | 0.000716286 | 0.021282911 | NR4A1, DLX5, CDH13, DPP4, SEMA5A, |
| | | regulation of cell | | | | | | | SHOX2, BMP2, PTPRC, TNFRSF11A, |
| | | proliferation | | | | | | | ITGB3, ADRA2A, CXCL5, IL6, NAMPT, NFATC2, FOSL2 |
| upregulated | GO: 0010604 | positive | 139 | 15462 | 2087 | 33 | 0.000725994 | 0.021359896 | FAM129A, IRF4, SHOX2, ADRA2A, |
| | | regulation of | | | | | | | TNNI2, HOXA5, NFATC2, NPAS2, NKD1, |
| | | macromolecule | | | | | | | GDF6, HIVEP3, ITGB3, RGCC, FOXO1, |
| | | metabolic | | | | | | | CNTN1, NR4A3, TLR1, MDFIC, CD36, |
| | | process | | | | | | | SATB2, LRRK2, BIRC3, NR4A1, DLX5, |
| | | | | | | | | | CDH13, BMP2, PTPRC, TNFRSF11A, |
| | | | | | | | | | WNT4, IL6, TBX5, NAMPT, ADIPOQ |
| upregulated | GO: 0032103 | positive | 139 | 15462 | 167 | 7 | 0.000781967 | 0.021709469 | TNFRSF11A, IL6, CDH13, ALOX5AP, |
| | | regulation of | | | | | | | SEMA5A, CCR1, FABP4 |
| | | response to | | | | | | | |
| | | external stimulus | | | | | | | |
| upregulated | GO: 0048812 | neuron | 139 | 15462 | 584 | 14 | 0.000786078 | 0.021709469 | NR4A3, LRRK2, NPTX1, DLX5, GFRA1, |
| | | projection | | | | | | | SDC2, DCLK1, SOS1, SEMA5A, SHOX2, |
| | | morphogenesis | | | | | | | PTPRC, ITGB3, PDLIM5, CNTN1 |
| upregulated | GO: 2000064 | regulation of | 139 | 15462 | 5 | 2 | 0.000788272 | 0.021709469 | WNT4, BMP2 |
| | | cortisol | | | | | | | |
| | | biosynthetic | | | | | | | |
| | | process | | | | | | | |
| upregulated | GO: 0071221 | cellular response | 139 | 15462 | 5 | 2 | 0.000788272 | 0.021709469 | TLR1, CD36 |
| | | to bacterial | | | | | | | |
| | | lipopeptide | | | | | | | |
| upregulated | GO: 0071220 | cellular response | 139 | 15462 | 5 | 2 | 0.000788272 | 0.021709469 | TLR1, CD36 |
| | | to bacterial | | | | | | | |
| | | lipoprotein | | | | | | | |
| upregulated | GO: 0070339 | response to | 139 | 15462 | 5 | 2 | 0.000788272 | 0.021709469 | TLR1, CD36 |
| | | bacterial | | | | | | | |
| | | lipopeptide | | | | | | | |
| upregulated | GO: 0050900 | leukocyte | 139 | 15462 | 272 | 9 | 0.000791305 | 0.021709469 | SOS1, CCR1, COL1A2, CD2, ITGB3, |
| | | migration | | | | | | | TNFRSF11A, IL6, SLC7A5, MMP1 |
| upregulated | GO: 0051270 | regulation of | 139 | 15462 | 518 | 13 | 0.000799703 | 0.021709469 | NKD1, CDH13, SEMA5A, CCR1, BMP2, |
| | | cellular | | | | | | | PTPRC, RGCC, ADRA2A, ITGB3, WNT4, |
| | | component | | | | | | | IL6, TBX5, ADIPOQ |
| | | movement | | | | | | | |
| upregulated | GO: 0009966 | regulation of | 139 | 15462 | 2099 | 33 | 0.000802983 | 0.021709469 | TAGAP, CBLC, IRF4, SHOX2, ADRA2A, |
| | | signal | | | | | | | NKD1, GDF6, IL18R1, RASGRF2, |
| | | transduction | | | | | | | DEPDC7, ITGB3, CXCL9, FOXO1, |
| | | | | | | | | | TSPAN5, SOS1, SEMA5A, MDFIC, |
| | | | | | | | | | PLA2R1, FEM1B, CD36, LRRK2, STAP1, |
| | | | | | | | | | BIRC3, DLX5, CDH13, BMP2, CCR1, |
| | | | | | | | | | PTPRC, TNFRSF11A, WNT4, IL6, SKAP2, ADIPOQ |
| upregulated | GO: 0022617 | extracellular | 139 | 15462 | 122 | 6 | 0.000815464 | 0.021850054 | ELN, COL12A1, DPP4, COL1A2, |
| | | matrix | | | | | | | ADAMTS5, MMP1 |
| | | disassembly | | | | | | | |
| upregulated | GO: 0042592 | homeostatic | 139 | 15462 | 1180 | 22 | 0.000829631 | 0.022032942 | STEAP4, CHRNA1, FABP4, ADRA2A, |
| | | process | | | | | | | HOXA5, IGJ, CALB1, LRRK2, GLRX, |
| | | | | | | | | | NPTX1, GCNT4, ANGPTL4, CCR1, |
| | | | | | | | | | CAMK2D, PTPRC, TNFRSF11A, ITGB3, |
| | | | | | | | | | IL6, ERCC1, CXCL9, ADIPOQ, FOXO1 |
| upregulated | GO: 0002675 | positive | 139 | 15462 | 21 | 3 | 0.000839803 | 0.022107452 | TNFRSF11A, IL6, ALOX5AP |
| | | regulation of | | | | | | | |
| | | acute | | | | | | | |
| | | inflammatory | | | | | | | |
| | | response | | | | | | | |
| upregulated | GO: 0043542 | endothelial cell | 139 | 15462 | 123 | 6 | 0.000851042 | 0.0222033 | RGCC, ITGB3, NR4A1, CDH13, DPP4, |
| | | migration | | | | | | | SEMA5A |
| upregulated | GO: 0001568 | blood vessel | 139 | 15462 | 522 | 13 | 0.000858242 | 0.0222033 | NR4A1, CDH13, ANGPTL4, SEMA5A, |
| | | development | | | | | | | COL1A2, RGCC, ITGB3, WNT4, IL6, |
| | | | | | | | | | HOXA5, TBX5, THSD7A, FOXO1 |
| upregulated | GO: 0007155 | cell adhesion | 139 | 15462 | 957 | 19 | 0.000930194 | 0.023859071 | SLAMF7, TENM3, SEMA5A, CD2, |
| | | | | | | | | | COL12A1, LSAMP, CD36, CDH13, |
| | | | | | | | | | MEGF10, DPP4, CCR1, BMP2, RGCC, |
| | | | | | | | | | ITGB3, WNT4, CPXM2, SIGLEC10, CNTN1, ADIPOQ |
| upregulated | GO: 0022610 | biological | 139 | 15462 | 959 | 19 | 0.000953505 | 0.024249745 | SLAMF7, TENM3, SEMA5A, CD2, |
| | | adhesion | | | | | | | COL12A1, LSAMP, CD36, CDH13, |
| | | | | | | | | | MEGF10, DPP4, CCR1, BMP2, RGCC, |
| | | | | | | | | | ITGB3, WNT4, CPXM2, SIGLEC10, CNTN1, ADIPOQ |
| upregulated | GO: 0032800 | receptor | 139 | 15462 | 22 | 3 | 0.000966034 | 0.024361908 | ITGB3, HOXA5, ADIPOQ |
| | | biosynthetic | | | | | | | |
| | | process | | | | | | | |
| upregulated | GO: 0050673 | epithelial cell | 139 | 15462 | 281 | 9 | 0.000995471 | 0.024895065 | NR4A1, DLX5, CDH13, SEMA5A, BMP2, |
| | | proliferation | | | | | | | ITGB3, RGCC, IL6, HOXA5 |
| upregulated | GO: 0051093 | negative | 139 | 15462 | 600 | 14 | 0.001019855 | 0.025294101 | NKD1, SEMA5A, CCR1, BMP2, RGCC, |
| | | regulation of | | | | | | | ITGB3, EFEMP1, WNT4, PRDM6, IL6, |
| | | developmental | | | | | | | HOXA5, TBX5, ADIPOQ, FOXO1 |
| | | process | | | | | | | |
| upregulated | GO: 0007173 | epidermal growth | 139 | 15462 | 227 | 8 | 0.00103415 | 0.025438403 | NR4A1, CBLC, CDH13, PDE1A, SOS1, |
| | | factor receptor | | | | | | | EFEMP1, ADRA2A, FOXO1 |
| | | signaling | | | | | | | |
| | | pathway | | | | | | | |
| upregulated | GO: 0007267 | cell-cell signaling | 139 | 15462 | 1122 | 21 | 0.001048771 | 0.025588301 | CHRNA1, SEMA5A, ADRA2A, HOXA5, |
| | | | | | | | | | PDLIM5, CALB1, LRRK2, NPTX1, |
| | | | | | | | | | RASGRF2, CAMK2D, BMP2, CCR1, |
| | | | | | | | | | TNFRSF11A, CXCL5, IL6, TBX5, NAMPT, |
| | | | | | | | | | ASIC2, CXCL9, BCHE, ADIPOQ |
| upregulated | GO: 0030334 | regulation of cell | 139 | 15462 | 469 | 12 | 0.001079656 | 0.026129416 | CDH13, SEMA5A, CCR1, BMP2, PTPRC, |
| | | migration | | | | | | | RGCC, ADRA2A, ITGB3, WNT4, IL6, TBX5, ADIPOQ |
| upregulated | GO: 1902533 | positive | 139 | 15462 | 605 | 14 | 0.001103969 | 0.026504087 | LRRK2, CDH13, SOS1, SEMA5A, CCR1, |
| | | regulation of | | | | | | | BMP2, TNFRSF11A, ADRA2A, IL6, |
| | | intracellular | | | | | | | PLA2R1, MDFIC, CXCL9, CD36, ADIPOQ |
| | | signal | | | | | | | |
| | | transduction | | | | | | | |
| upregulated | GO: 0044236 | multicellular | 139 | 15462 | 130 | 6 | 0.001134807 | 0.026760646 | RGCC, WNT4, COL12A1, IL6, MMP1, |
| | | organismal | | | | | | | COL1A2 |
| | | metabolic | | | | | | | |
| | | process | | | | | | | |
| upregulated | GO: 0006955 | immune | 139 | 15462 | 1292 | 23 | 0.001168924 | 0.026760646 | SLAMF7, TLR1, IRF4, SOS1, IGJ, |
| | | response | | | | | | | NFATC2, CD36, BIRC3, NR4A1, PDE1A, |
| | | | | | | | | | IL18R1, CCR1, FYB, CAMK2D, PTPRC, |
| | | | | | | | | | TNFRSF11A, RGCC, CXCL5, LCP2, IL6, |
| | | | | | | | | | ERCC1, CXCL9, FOXO1 |
| upregulated | GO: 0055098 | response to low- | 139 | 15462 | 6 | 2 | 0.001175437 | 0.026760646 | CDH13, CD36 |
| | | density | | | | | | | |
| | | lipoprotein | | | | | | | |
| | | particle | | | | | | | |
| upregulated | GO: 0032493 | response to | 139 | 15462 | 6 | 2 | 0.001175437 | 0.026760646 | TLR1, CD36 |
| | | bacterial | | | | | | | |
| | | lipoprotein | | | | | | | |
| upregulated | GO: 0032347 | regulation of | 139 | 15462 | 6 | 2 | 0.001175437 | 0.026760646 | WNT4, BMP2 |
| | | aldosterone | | | | | | | |
| | | biosynthetic | | | | | | | |
| | | process | | | | | | | |
| upregulated | GO: 0010871 | negative | 139 | 15462 | 6 | 2 | 0.001175437 | 0.026760646 | ITGB3, ADIPOQ |
| | | regulation of | | | | | | | |
| | | receptor | | | | | | | |
| | | biosynthetic | | | | | | | |
| | | process | | | | | | | |
| upregulated | GO: 0010628 | positive | 139 | 15462 | 1212 | 22 | 0.001177076 | 0.026760646 | NR4A3, IRF4, SHOX2, TNNI2, MDFIC, |
| | | regulation of | | | | | | | HOXA5, NFATC2, SATB2, NPAS2, |
| | | gene expression | | | | | | | NR4A1, GDF6, DLX5, CDH13, HIVEP3, |
| | | | | | | | | | BMP2, RGCC, WNT4, IL6, TBX5, NAMPT, FOXO1, CNTN1 |
| upregulated | GO: 0038127 | ERBB signaling | 139 | 15462 | 232 | 8 | 0.001189141 | 0.02683168 | NR4A1, CBLC, CDH13, PDE1A, SOS1, |
| | | pathway | | | | | | | EFEMP1, ADRA2A, FOXO1 |
| upregulated | GO: 0050789 | regulation of | 139 | 15462 | 9283 | 101 | 0.001231664 | 0.027425621 | TAGAP, FAM129A, P2RY8, SHOX2, |
| | | biological | | | | | | | IRF4, FABP4, TNNI2, RAB30, BNIP3L, |
| | | process | | | | | | | TFEC, NPAS2, GRP, PNMA2, COL1A2, |
| | | | | | | | | | LCP2, ASIC2, BCHE, FOXO1, |
| | | | | | | | | | ARHGAP42, CNTN1, TSPAN5, TLR1, |
| | | | | | | | | | TENM3, CHRNA1, SOS1, MDFIC, |
| | | | | | | | | | PLA2R1, FEM1B, ALOX5AP, FOSL2, |
| | | | | | | | | | CD36, SATB2, ZFHX4, GPR34, PRKG1, |
| | | | | | | | | | PDE1A, GUCY1A3, TNFRSF11A, IL6, |
| | | | | | | | | | TBX5, GPC6, CBLC, DCLK1, CD2, |
| | | | | | | | | | ADRA2A, HOXA5, IGJ, NFATC2, PDLIM5, |
| | | | | | | | | | NKD1, GDF6, CELF2, GLRX, GFRA1, |
| | | | | | | | | | RASGRF2, IL18R1, DPP4, DEPDC7, |
| | | | | | | | | | DHX34, CAMK2D, HIVEP3, FYB, RGCC, |
| | | | | | | | | | ITGB3, PRDM6, BNC2, ERCC1, TFPI2, |
| | | | | | | | | | CXCL9, NR4A3, FPR3, SLAMF7, |
| | | | | | | | | | SEMA5A, RUNX1T1, RFX2, CLEC1A, |
| | | | | | | | | | EFEMP1, PPP1R3B, MCTP1, DOCK8, |
| | | | | | | | | | CALB1, ELN, FBXO32, LRRK2, BIRC3, |
| | | | | | | | | | STAP1, NR4A1, DLX5, CDH13, SDC2, |
| | | | | | | | | | MEGF10, ANGPTL4, PTPRC, CCR1, |
| | | | | | | | | | BMP2, WNT4, CXCL5, RARRES1, |
| | | | | | | | | | NAMPT, SKAP2, ADIPOQ |
| upregulated | GO: 0010646 | regulation of cell | 139 | 15462 | 2333 | 35 | 0.001251576 | 0.027425621 | TAGAP, CBLC, IRF4, SHOX2, ADRA2A, |
| | | communication | | | | | | | NKD1, GDF6, IL18R1, RASGRF2, |
| | | | | | | | | | DEPDC7, ITGB3, CXCL9, BCHE, FOXO1, |
| | | | | | | | | | TSPAN5, SOS1, SEMA5A, MDFIC, |
| | | | | | | | | | PLA2R1, FEM1B, CD36, CALB1, LRRK2, |
| | | | | | | | | | STAP1, BIRC3, DLX5, CDH13, BMP2, |
| | | | | | | | | | CCR1, PTPRC, TNFRSF11A, WNT4, IL6, SKAP2, ADIPOQ |
| upregulated | GO: 0003170 | heart valve | 139 | 15462 | 24 | 3 | 0.001253074 | 0.027425621 | TBX5, SHOX2, BMP2 |
| | | development | | | | | | | |
| upregulated | GO: 0010769 | regulation of cell | 139 | 15462 | 234 | 8 | 0.001256102 | 0.027425621 | LRRK2, SDC2, SHOX2, SEMA5A, BMP2, |
| | | morphogenesis | | | | | | | RGCC, TBX5, PDLIM5 |
| | | involved in | | | | | | | |
| | | differentiation | | | | | | | |
| upregulated | GO: 0023051 | regulation of | 139 | 15462 | 2334 | 35 | 0.001261158 | 0.027425621 | TAGAP, CBLC, IRF4, SHOX2, ADRA2A, |
| | | signaling | | | | | | | NKD1, GDF6, IL18R1, RASGRF2, |
| | | | | | | | | | DEPDC7, ITGB3, CXCL9, BCHE, FOXO1, |
| | | | | | | | | | TSPAN5, SOS1, SEMA5A, MDFIC, |
| | | | | | | | | | PLA2R1, FEM1B, CD36, CALB1, LRRK2, |
| | | | | | | | | | STAP1, BIRC3, DLX5, CDH13, BMP2, |
| | | | | | | | | | CCR1, PTPRC, TNFRSF11A, WNT4, IL6, |
| | | | | | | | | | SKAP2, ADIPOQ |
| upregulated | GO: 0071310 | cellular response | 139 | 15462 | 1634 | 27 | 0.00128323 | 0.027704834 | IRF4, SOS1, ADRA2A, FOSL2, CD36, |
| | | to organic | | | | | | | CALB1, SATB2, LRRK2, NR4A1, NPTX1, |
| | | substance | | | | | | | DLX5, RASGRF2, PDE1A, CDH13, |
| | | | | | | | | | IL18R1, CCR1, COL1A2, CAMK2D, |
| | | | | | | | | | PTPRC, BMP2, TNFRSF11A, ITGB3, |
| | | | | | | | | | WNT4, IL6, NAMPT, ADIPOQ, FOXO1 |
| upregulated | GO: 0006029 | proteoglycan | 139 | 15462 | 90 | 5 | 0.001301884 | 0.027906805 | GPC6, CHSY3, SDC2, BMP2, HS3ST3B1 |
| | | metabolic | | | | | | | |
| | | process | | | | | | | |
| upregulated | GO: 0001944 | vasculature | 139 | 15462 | 548 | 13 | 0.001333538 | 0.028382606 | NR4A1, CDH13, ANGPTL4, SEMA5A, |
| | | development | | | | | | | COL1A2, RGCC, ITGB3, WNT4, IL6, |
| | | | | | | | | | HOXA5, TBX5, THSD7A, FOXO1 |
| upregulated | GO: 0010557 | positive | 139 | 15462 | 1306 | 23 | 0.001348983 | 0.028509136 | FAM129A, NR4A3, TLR1, IRF4, SHOX2, |
| | | regulation of | | | | | | | TNNI2, MDFIC, HOXA5, NFATC2, SATB2, |
| | | macromolecule | | | | | | | NPAS2, NR4A1, GDF6, DLX5, CDH13, |
| | | biosynthetic | | | | | | | HIVEP3, BMP2, RGCC, WNT4, IL6, TBX5, |
| | | process | | | | | | | NAMPT, FOXO1 |
| upregulated | GO: 0032101 | regulation of | 139 | 15462 | 482 | 12 | 0.001362888 | 0.028601591 | BIRC3, CDH13, SEMA5A, CCR1, FABP4, |
| | | response to | | | | | | | TNFRSF11A, WNT4, IL6, ASIC2, |
| | | external stimulus | | | | | | | ALOX5AP, CD36, ADIPOQ |
| upregulated | GO: 0060326 | cell chemotaxis | 139 | 15462 | 184 | 7 | 0.001375394 | 0.028663592 | TNFRSF11A, CXCL5, IL6, NR4A1, |
| | | | | | | | | | CXCL9, SEMA5A, CCR1 |
| upregulated | GO: 1903036 | positive | 139 | 15462 | 92 | 5 | 0.001436074 | 0.02972178 | ADRA2A, TNFRSF11A, IL6, ALOX5AP, |
| | | regulation of | | | | | | | FABP4 |
| | | response to | | | | | | | |
| | | wounding | | | | | | | |
| upregulated | GO: 0032965 | regulation of | 139 | 15462 | 26 | 3 | 0.00158869 | 0.031673473 | RGCC, WNT4, IL6 |
| | | collagen | | | | | | | |
| | | biosynthetic | | | | | | | |
| | | process | | | | | | | |
| upregulated | GO: 0031328 | positive | 139 | 15462 | 1407 | 24 | 0.001620828 | 0.031673473 | FAM129A, NR4A3, TLR1, IRF4, SHOX2, |
| | | regulation of | | | | | | | TNNI2, MDFIC, HOXA5, NFATC2, SATB2, |
| | | cellular | | | | | | | NPAS2, NR4A1, GDF6, DLX5, CDH13, |
| | | biosynthetic | | | | | | | HIVEP3, GUCY1A3, BMP2, RGCC, |
| | | process | | | | | | | WNT4, IL6, TBX5, NAMPT, FOXO1 |
| upregulated | GO: 0010566 | regulation of | 139 | 15462 | 7 | 2 | 0.001635917 | 0.031673473 | WNT4, BMP2 |
| | | ketone | | | | | | | |
| | | biosynthetic | | | | | | | |
| | | process | | | | | | | |
| upregulated | GO: 0072540 | T-helper 17 cell | 139 | 15462 | 7 | 2 | 0.001635917 | 0.031673473 | IL6, IRF4 |
| | | lineage | | | | | | | |
| | | commitment | | | | | | | |
| upregulated | GO: 0031946 | regulation of | 139 | 15462 | 7 | 2 | 0.001635917 | 0.031673473 | WNT4, BMP2 |
| | | glucocorticoid | | | | | | | |
| | | biosynthetic | | | | | | | |
| | | process | | | | | | | |
| upregulated | GO: 0045714 | regulation of low- | 139 | 15462 | 7 | 2 | 0.001635917 | 0.031673473 | ITGB3, ADIPOQ |
| | | density | | | | | | | |
| | | lipoprotein | | | | | | | |
| | | particle receptor | | | | | | | |
| | | biosynthetic | | | | | | | |
| | | process | | | | | | | |
| upregulated | GO: 1901522 | positive | 139 | 15462 | 7 | 2 | 0.001635917 | 0.031673473 | DLX5, BMP2 |
| | | regulation of | | | | | | | |
| | | transcription | | | | | | | |
| | | from RNA | | | | | | | |
| | | polymerase II | | | | | | | |
| | | promoter | | | | | | | |
| | | involved in | | | | | | | |
| | | cellular response | | | | | | | |
| | | to chemical | | | | | | | |
| | | stimulus | | | | | | | |
| upregulated | GO: 0032353 | negative | 139 | 15462 | 7 | 2 | 0.001635917 | 0.031673473 | WNT4, BMP2 |
| | | regulation of | | | | | | | |
| | | hormone | | | | | | | |
| | | biosynthetic | | | | | | | |
| | | process | | | | | | | |
| upregulated | GO: 0038030 | non-canonical | 139 | 15462 | 7 | 2 | 0.001635917 | 0.031673473 | WNT4, NKD1 |
| | | Wnt signaling | | | | | | | |
| | | pathway via | | | | | | | |
| | | MAPK cascade | | | | | | | |
| upregulated | GO: 0032344 | regulation of | 139 | 15462 | 7 | 2 | 0.001635917 | 0.031673473 | WNT4, BMP2 |
| | | aldosterone | | | | | | | |
| | | metabolic | | | | | | | |
| | | process | | | | | | | |
| upregulated | GO: 0007162 | negative | 139 | 15462 | 96 | 5 | 0.001734679 | 0.033370338 | RGCC, CDH13, SEMA5A, ADIPOQ, |
| | | regulation of cell | | | | | | | BMP2 |
| | | adhesion | | | | | | | |
| upregulated | GO: 0022408 | negative | 139 | 15462 | 27 | 3 | 0.001775592 | 0.033939815 | RGCC, ADIPOQ, BMP2 |
| | | regulation of cell- | | | | | | | |
| | | cell adhesion | | | | | | | |
| upregulated | GO: 0033002 | muscle cell | 139 | 15462 | 97 | 5 | 0.001815946 | 0.034389893 | IL6, TBX5, CDH13, PDE1A, ADIPOQ |
| | | proliferation | | | | | | | |
| upregulated | GO: 0045669 | positive | 139 | 15462 | 58 | 4 | 0.001822057 | 0.034389893 | WNT4, IL6, DLX5, BMP2 |
| | | regulation of | | | | | | | |
| | | osteoblast | | | | | | | |
| | | differentiation | | | | | | | |
| upregulated | GO: 0045935 | positive | 139 | 15462 | 1339 | 23 | 0.001870772 | 0.035088672 | NR4A3, IRF4, SHOX2, TNNI2, MDFIC, |
| | | regulation of | | | | | | | HOXA5, NFATC2, SATB2, NPAS2, |
| | | nucleobase- | | | | | | | NR4A1, GDF6, DLX5, CDH13, HIVEP3, |
| | | containing | | | | | | | GUCY1A3, BMP2, RGCC, WNT4, IL6, |
| | | compound | | | | | | | TBX5, NAMPT, CXCL9, FOXO1 |
| | | metabolic | | | | | | | |
| | | process | | | | | | | |
| upregulated | GO: 0050794 | regulation of | 139 | 15462 | 8782 | 96 | 0.001938017 | 0.035923289 | TAGAP, FAM129A, P2RY8, SHOX2, |
| | | cellular process | | | | | | | IRF4, FABP4, TNNI2, RAB30, BNIP3L, |
| | | | | | | | | | TFEC, NPAS2, GRP, PNMA2, COL1A2, |
| | | | | | | | | | LCP2, ASIC2, BCHE, FOXO1, |
| | | | | | | | | | ARHGAP42, CNTN1, TSPAN5, TLR1, |
| | | | | | | | | | TENM3, CHRNA1, SOS1, MDFIC, |
| | | | | | | | | | PLA2R1, FEM1B, FOSL2, CD36, SATB2, |
| | | | | | | | | | ZFHX4, GPR34, PRKG1, PDE1A, |
| | | | | | | | | | GUCY1A3, TNFRSF11A, IL6, TBX5, |
| | | | | | | | | | GPC6, CBLC, DCLK1, CD2, ADRA2A, |
| | | | | | | | | | HOXA5, NFATC2, PDLIM5, NKD1, GDF6, |
| | | | | | | | | | GLRX, GFRA1, RASGRF2, IL18R1, |
| | | | | | | | | | DPP4, DEPDC7, DHX34, CAMK2D, |
| | | | | | | | | | HIVEP3, FYB, RGCC, ITGB3, PRDM6, |
| | | | | | | | | | BNC2, ERCC1, CXCL9, NR4A3, FPR3, |
| | | | | | | | | | SLAMF7, SEMA5A, RUNX1T1, RFX2, |
| | | | | | | | | | CLEC1A, EFEMP1, PPP1R3B, MCTP1, |
| | | | | | | | | | DOCK8, CALB1, ELN, LRRK2, BIRC3, |
| | | | | | | | | | STAP1, NR4A1, DLX5, CDH13, SDC2, |
| | | | | | | | | | MEGF10, ANGPTL4, PTPRC, CCR1, |
| | | | | | | | | | BMP2, WNT4, CXCL5, RARRES1, |
| | | | | | | | | | NAMPT, SKAP2, ADIPOQ |
| upregulated | GO: 0048666 | neuron | 139 | 15462 | 866 | 17 | 0.001974063 | 0.035923289 | NR4A3, TENM3, DCLK1, SOS1, |
| | | development | | | | | | | SEMA5A, SHOX2, PDLIM5, LRRK2, |
| | | | | | | | | | PRKG1, GFRA1, DLX5, NPTX1, SDC2, |
| | | | | | | | | | PTPRC, ITGB3, IL6, CNTN1 |
| upregulated | GO: 0010743 | regulation of | 139 | 15462 | 28 | 3 | 0.001975667 | 0.035923289 | ITGB3, CD36, ADIPOQ |
| | | macrophage | | | | | | | |
| | | derived foam cell | | | | | | | |
| | | differentiation | | | | | | | |
| upregulated | GO: 0046649 | lymphocyte | 139 | 15462 | 504 | 12 | 0.001981399 | 0.035923289 | SLAMF7, IL18R1, IRF4, DPP4, PTPRC, |
| | | activation | | | | | | | CD2, WNT4, IL6, ERCC1, NFATC2, |
| | | | | | | | | | SKAP2, DOCK8 |
| upregulated | GO: 0007519 | skeletal muscle | 139 | 15462 | 145 | 6 | 0.001982918 | 0.035923289 | ELN, NR4A1, MEGF10, SHOX2, |
| | | tissue | | | | | | | CHRNA1, HIVEP3 |
| | | development | | | | | | | |
| upregulated | GO: 0009891 | positive | 139 | 15462 | 1429 | 24 | 0.001994951 | 0.035923289 | FAM129A, NR4A3, TLR1, IRF4, SHOX2, |
| | | regulation of | | | | | | | TNNI2, MDFIC, HOXA5, NFATC2, SATB2, |
| | | biosynthetic | | | | | | | NPAS2, NR4A1, GDF6, DLX5, CDH13, |
| | | process | | | | | | | HIVEP3, GUCY1A3, BMP2, RGCC, |
| | | | | | | | | | WNT4, IL6, TBX5, NAMPT, FOXO1 |
| upregulated | GO: 0000902 | cell | 139 | 15462 | 944 | 18 | 0.001999063 | 0.035923289 | NR4A3, DCLK1, SOS1, SEMA5A, |
| | | morphogenesis | | | | | | | SHOX2, PDLIM5, LRRK2, GFRA1, DLX5, |
| | | | | | | | | | NPTX1, SDC2, PTPRC, BMP2, RGCC, |
| | | | | | | | | | ITGB3, WNT4, TBX5, CNTN1 |
| upregulated | GO: 0045664 | regulation of | 139 | 15462 | 374 | 10 | 0.002042815 | 0.036051073 | LRRK2, GDF6, SDC2, TENM3, SEMA5A, |
| | | neuron | | | | | | | SHOX2, BMP2, IL6, PDLIM5, CNTN1 |
| | | differentiation | | | | | | | |
| upregulated | GO: 0009887 | organ | 139 | 15462 | 719 | 15 | 0.002048118 | 0.036051073 | ELN, LRRK2, NKD1, DLX5, GCNT4, |
| | | morphogenesis | | | | | | | TENM3, SHOX2, BMP2, COL1A2, WNT4, |
| | | | | | | | | | HOXA5, FEM1B, TBX5, CALB1, SATB2 |
| upregulated | GO: 0060538 | skeletal muscle | 139 | 15462 | 146 | 6 | 0.002052896 | 0.036051073 | ELN, NR4A1, MEGF10, SHOX2, |
| | | organ | | | | | | | CHRNA1, HIVEP3 |
| | | development | | | | | | | |
| upregulated | GO: 0022604 | regulation of cell | 139 | 15462 | 253 | 8 | 0.002054226 | 0.036051073 | LRRK2, SDC2, SHOX2, SEMA5A, BMP2, |
| | | morphogenesis | | | | | | | RGCC, TBX5, PDLIM5 |
| upregulated | GO: 1902531 | regulation of | 139 | 15462 | 1268 | 22 | 0.002090967 | 0.036482508 | TAGAP, CBLC, SOS1, SEMA5A, |
| | | intracellular | | | | | | | ADRA2A, MDFIC, PLA2R1, CD36, |
| | | signal | | | | | | | LRRK2, BIRC3, CDH13, IL18R1, |
| | | transduction | | | | | | | RASGRF2, DEPDC7, CCR1, BMP2, |
| | | | | | | | | | TNFRSF11A, WNT4, IL6, CXCL9, |
| | | | | | | | | | ADIPOQ, FOXO1 |
| upregulated | GO: 0034650 | cortisol | 139 | 15462 | 8 | 2 | 0.002168382 | 0.036557948 | WNT4, BMP2 |
| | | metabolic | | | | | | | |
| | | process | | | | | | | |
| upregulated | GO: 0045713 | low-density | 139 | 15462 | 8 | 2 | 0.002168382 | 0.036557948 | ITGB3, ADIPOQ |
| | | lipoprotein | | | | | | | |
| | | particle receptor | | | | | | | |
| | | biosynthetic | | | | | | | |
| | | process | | | | | | | |
| upregulated | GO: 0055094 | response to | 139 | 15462 | 8 | 2 | 0.002168382 | 0.036557948 | CDH13, CD36 |
| | | lipoprotein | | | | | | | |
| | | particle | | | | | | | |
| upregulated | GO: 0034651 | cortisol | 139 | 15462 | 8 | 2 | 0.002168382 | 0.036557948 | WNT4, BMP2 |
| | | biosynthetic | | | | | | | |
| | | process | | | | | | | |
| upregulated | GO: 0032351 | negative | 139 | 15462 | 8 | 2 | 0.002168382 | 0.036557948 | WNT4, BMP2 |
| | | regulation of | | | | | | | |
| | | hormone | | | | | | | |
| | | metabolic | | | | | | | |
| | | process | | | | | | | |
| upregulated | GO: 0006910 | phagocytosis, | 139 | 15462 | 8 | 2 | 0.002168382 | 0.036557948 | MEGF10, CD36 |
| | | recognition | | | | | | | |
| upregulated | GO: 0010712 | regulation of | 139 | 15462 | 29 | 3 | 0.002189235 | 0.036703316 | RGCC, WNT4, IL6 |
| | | collagen | | | | | | | |
| | | metabolic | | | | | | | |
| | | process | | | | | | | |
| upregulated | GO: 0045893 | positive | 139 | 15462 | 1112 | 20 | 0.002232572 | 0.037221934 | NR4A3, IRF4, SHOX2, TNNI2, MDFIC, |
| | | regulation of | | | | | | | HOXA5, NFATC2, SATB2, NPAS2, |
| | | transcription, | | | | | | | NR4A1, GDF6, DLX5, CDH13, HIVEP3, |
| | | DNA-templated | | | | | | | BMP2, WNT4, IL6, TBX5, NAMPT, |
| | | | | | | | | | FOXO1 |
| upregulated | GO: 0010941 | regulation of cell | 139 | 15462 | 1276 | 22 | 0.002261453 | 0.037337936 | NR4A3, SOS1, SEMA5A, HOXA5, |
| | | death | | | | | | | FEM1B, BNIP3L, LRRK2, NPAS2, BIRC3, |
| | | | | | | | | | NR4A1, RASGRF2, PDE1A, ANGPTL4, |
| | | | | | | | | | PNMA2, BMP2, RGCC, WNT4, IL6, TBX5, |
| | | | | | | | | | ASIC2, FOXO1, ADIPOQ |
| upregulated | GO: 0014706 | striated muscle | 139 | 15462 | 257 | 8 | 0.002264413 | 0.037337936 | ELN, NR4A1, MEGF10, CHRNA1, |
| | | tissue | | | | | | | SHOX2, BMP2, HIVEP3, TBX5 |
| | | development | | | | | | | |
| upregulated | GO: 0000165 | MAPK cascade | 139 | 15462 | 583 | 13 | 0.002303419 | 0.037773554 | LRRK2, NKD1, CBLC, CCR1, BMP2, |
| | | | | | | | | | TNFRSF11A, ADRA2A, WNT4, IL6, |
| | | | | | | | | | MDFIC, CD36, ADIPOQ, FOXO1 |
| upregulated | GO: 0051173 | positive | 139 | 15462 | 1362 | 23 | 0.002329571 | 0.037994796 | NR4A3, IRF4, SHOX2, TNNI2, MDFIC, |
| | | regulation of | | | | | | | HOXA5, NFATC2, SATB2, NPAS2, |
| | | nitrogen | | | | | | | NR4A1, GDF6, DLX5, CDH13, HIVEP3, |
| | | compound | | | | | | | GUCY1A3, BMP2, RGCC, WNT4, IL6, |
| | | metabolic | | | | | | | TBX5, NAMPT, CXCL9, FOXO1 |
| | | process | | | | | | | |
| upregulated | GO: 0019220 | regulation of | 139 | 15462 | 1705 | 27 | 0.002381057 | 0.038624607 | TAGAP, FAM129A, CBLC, SOS1, FABP4, |
| | | phosphate | | | | | | | ADRA2A, MDFIC, CD36, DOCK8, LRRK2, |
| | | metabolic | | | | | | | BIRC3, GDF6, PRKG1, RASGRF2, CCR1, |
| | | process | | | | | | | BMP2, GUCY1A3, PTPRC, TNFRSF11A, |
| | | | | | | | | | ITGB3, RGCC, WNT4, IL6, CXCL9, |
| | | | | | | | | | ADIPOQ, FOXO1, ARHGAP42 |
| upregulated | GO: 0002548 | monocyte | 139 | 15462 | 30 | 3 | 0.002416601 | 0.038781915 | TNFRSF11A, IL6, CCR1 |
| | | chemotaxis | | | | | | | |
| upregulated | GO: 2000351 | regulation of | 139 | 15462 | 30 | 3 | 0.002416601 | 0.038781915 | RGCC, SEMA5A, ANGPTL4 |
| | | endothelial cell | | | | | | | |
| | | apoptotic | | | | | | | |
| | | process | | | | | | | |
| upregulated | GO: 0032386 | regulation of | 139 | 15462 | 320 | 9 | 0.002429793 | 0.038786217 | LRRK2, IL18R1, SEMA5A, CAMK2D, IL6, |
| | | intracellular | | | | | | | MDFIC, CXCL9, CD36, ADIPOQ |
| | | transport | | | | | | | |
| upregulated | GO: 0051223 | regulation of | 139 | 15462 | 322 | 9 | 0.002533423 | 0.040226465 | TLR1, IL18R1, SEMA5A, CD2, RGCC, |
| | | protein transport | | | | | | | IL6, MDFIC, CD36, ADIPOQ |
| upregulated | GO: 0060284 | regulation of cell | 139 | 15462 | 590 | 13 | 0.002554323 | 0.04034486 | LRRK2, GDF6, SDC2, TENM3, SEMA5A, |
| | | development | | | | | | | SHOX2, BMP2, RGCC, IL6, TBX5, |
| | | | | | | | | | PDLIM5, ADIPOQ, CNTN1 |
| upregulated | GO: 0051051 | negative | 139 | 15462 | 323 | 9 | 0.002586539 | 0.040639812 | LRRK2, RGCC, ITGB3, ADRA2A, IL6, |
| | | regulation of | | | | | | | PLA2R1, MDFIC, CD36, ADIPOQ |
| | | transport | | | | | | | |
| upregulated | GO: 0048729 | tissue | 139 | 15462 | 453 | 11 | 0.002615755 | 0.04073483 | NR4A3, NKD1, GCNT4, SEMA5A, |
| | | morphogenesis | | | | | | | SHOX2, BMP2, WNT4, IL6, HOXA5, |
| | | | | | | | | | TBX5, FEM1B |
| upregulated | GO: 0051174 | regulation of | 139 | 15462 | 1718 | 27 | 0.002651998 | 0.04073483 | TAGAP, FAM129A, CBLC, SOS1, FABP4, |
| | | phosphorus | | | | | | | ADRA2A, MDFIC, CD36, DOCK8, LRRK2, |
| | | metabolic | | | | | | | BIRC3, GDF6, PRKG1, RASGRF2, CCR1, |
| | | process | | | | | | | BMP2, GUCY1A3, PTPRC, TNFRSF11A, |
| | | | | | | | | | ITGB3, RGCC. WNT4, IL6, CXCL9, |
| | | | | | | | | | ADIPOQ, FOXO1, ARHGAP42 |
| upregulated | GO: 0090077 | foam cell | 139 | 15462 | 31 | 3 | 0.00265806 | 0.04073483 | ITGB3, CD36, ADIPOQ |
| | | differentiation | | | | | | | |
| upregulated | GO: 0010742 | macrophage | 139 | 15462 | 31 | 3 | 0.00265806 | 0.04073483 | ITGB3, CD36, ADIPOQ |
| | | derived foam cell | | | | | | | |
| | | differentiation | | | | | | | |
| upregulated | GO: 0048514 | blood vessel | 139 | 15462 | 454 | 11 | 0.002660455 | 0.04073483 | NR4A1, CDH13, SEMA5A, ANGPTL4, |
| | | morphogenesis | | | | | | | RGCC, ITGB3, WNT4, IL6, HOXA5, |
| | | | | | | | | | THSD7A, TBX5 |
| upregulated | GO: 0031349 | positive | 139 | 15462 | 265 | 8 | 0.002735354 | 0.041586635 | TNFRSF11A, BIRC3, IL6, TLR1, |
| | | regulation of | | | | | | | ALOX5AP, IRF4, CD36, FABP4 |
| | | defense | | | | | | | |
| | | response | | | | | | | |
| upregulated | GO: 0031943 | regulation of | 139 | 15462 | 9 | 2 | 0.002771519 | 0.041586635 | WNT4, BMP2 |
| | | glucocorticoid | | | | | | | |
| | | metabolic | | | | | | | |
| | | process | | | | | | | |
| upregulated | GO: 0045721 | negative | 139 | 15462 | 9 | 2 | 0.002771519 | 0.041586635 | IL6, ADIPOQ |
| | | regulation of | | | | | | | |
| | | gluconeogenesis | | | | | | | |
| upregulated | GO: 0032342 | aldosterone | 139 | 15462 | 9 | 2 | 0.002771519 | 0.041586635 | WNT4, BMP2 |
| | | biosynthetic | | | | | | | |
| | | process | | | | | | | |
| upregulated | GO: 0030203 | glycosaminoglycan | 139 | 15462 | 156 | 6 | 0.002859745 | 0.042656604 | GPC6, CHSY3, PRELP, SDC2, GALNT5, |
| | | metabolic | | | | | | | HS3ST3B1 |
| | | process | | | | | | | |
| upregulated | GO: 0042221 | response to | 139 | 15462 | 3399 | 45 | 0.002903186 | 0.042656604 | IRF4, FABP4, ADRA2A, NFATC2, NPTX1, |
| | | chemical | | | | | | | GFRA1, IL18R1, RASGRF2, COL1A2, |
| | | | | | | | | | CAMK2D, ITGB3, RGCC, ERCC1, ASIC2, |
| | | | | | | | | | ADH1B, CXCL9, BCHE, FOXO1, CNTN1, |
| | | | | | | | | | PAPPA, NR4A3, FPR3, TLR1, SOS1, |
| | | | | | | | | | SEMA5A, PLA2R1, ALOX5AP, FOSL2, |
| | | | | | | | | | CD36, CALB1, SATB2, LRRK2, NR4A1, |
| | | | | | | | | | DLX5, CDH13, PDE1A, PTPRC, BMP2, |
| | | | | | | | | | CCR1, TNFRSF11A, CXCL5, WNT4, IL6, |
| | | | | | | | | | NAMPT, ADIPOQ |
| upregulated | GO: 0050663 | cytokine | 139 | 15462 | 108 | 5 | 0.002904014 | 0.042656604 | RGCC, CD2, LCP2, IL6, TLR1 |
| | | secretion | | | | | | | |
| upregulated | GO: 0032964 | collagen | 139 | 15462 | 32 | 3 | 0.002913897 | 0.042656604 | RGCC, WNT4, IL6 |
| | | biosynthetic | | | | | | | |
| | | process | | | | | | | |
| upregulated | GO: 0001954 | positive | 139 | 15462 | 32 | 3 | 0.002913897 | 0.042656604 | WNT4, CDH13, CD36 |
| | | regulation of cell- | | | | | | | |
| | | matrix adhesion | | | | | | | |
| upregulated | GO: 0006022 | aminoglycan | 139 | 15462 | 158 | 6 | 0.003046131 | 0.044031472 | GPC6, CHSY3, PRELP, SDC2, GALNT5, |
| | | metabolic | | | | | | | HS3ST3B1 |
| | | process | | | | | | | |
| upregulated | GO: 0030168 | platelet | 139 | 15462 | 212 | 7 | 0.003061618 | 0.044031472 | ADRA2A, ITGB3, LCP2, IL6, SOS1, |
| | | activation | | | | | | | CD36, COL1A2 |
| upregulated | GO: 0030335 | positive | 139 | 15462 | 270 | 8 | 0.003066504 | 0.044031472 | CDH13, SEMA5A, CCR1, BMP2, PTPRC, |
| | | regulation of cell | | | | | | | ITGB3, ADRA2A, IL6 |
| | | migration | | | | | | | |
| upregulated | GO: 0060537 | muscle tissue | 139 | 15462 | 270 | 8 | 0.003066504 | 0.044031472 | ELN, NR4A1, MEGF10, CHRNA1, |
| | | development | | | | | | | SHOX2, BMP2, HIVEP3, TBX5 |
| upregulated | GO: 0043067 | regulation of | 139 | 15462 | 1227 | 21 | 0.003125952 | 0.044671343 | NR4A3, SOS1, SEMA5A, HOXA5, |
| | | programmed cell | | | | | | | FEM1B, BNIP3L, LRRK2, BIRC3, NR4A1, |
| | | death | | | | | | | RASGRF2, PDE1A, ANGPTL4, PNMA2, |
| | | | | | | | | | BMP2, RGCC, WNT4, IL6, TBX5, ASIC2, |
| | | | | | | | | | FOXO1, ADIPOQ |
| upregulated | GO: 0072577 | endothelial cell | 139 | 15462 | 33 | 3 | 0.003184384 | 0.045265421 | RGCC, SEMA5A, ANGPTL4 |
| | | apoptotic | | | | | | | |
| | | process | | | | | | | |
| upregulated | GO: 0001501 | skeletal system | 139 | 15462 | 398 | 10 | 0.003197691 | 0.045265421 | PRELP, DLX5, SHOX2, BMP2, PTPRC, |
| | | development | | | | | | | COL1A2, EFEMP1, COL12A1, HOXA5, |
| | | | | | | | | | SATB2 |
| upregulated | GO: 0042058 | regulation of | 139 | 15462 | 68 | 4 | 0.003264038 | 0.045987687 | ADRA2A, CBLC, CDH13, SOS1 |
| | | epidermal growth | | | | | | | |
| | | factor receptor | | | | | | | |
| | | signaling | | | | | | | |
| | | pathway | | | | | | | |
| upregulated | GO: 0006954 | inflammatory | 139 | 15462 | 537 | 12 | 0.003326775 | 0.046140796 | BIRC3, TLR1, AOX1, CCR1, BMP2, |
| | | response | | | | | | | FABP4, TNFRSF11A, ADRA2A, IL6, |
| | | | | | | | | | CXCL9, ALOX5AP, ADIPOQ |
| upregulated | GO: 0051247 | positive | 139 | 15462 | 990 | 18 | 0.003335043 | 0.046140796 | FAM129A, TLR1, IRF4, ADRA2A, MDFIC, |
| | | regulation of | | | | | | | CD36, LRRK2, BIRC3, NKD1, GDF6, |
| | | protein metabolic | | | | | | | PTPRC, BMP2, RGCC, TNFRSF11A, |
| | | process | | | | | | | ITGB3, IL6, FOXO1, ADIPOQ |
| upregulated | GO: 1902680 | positive | 139 | 15462 | 1152 | 20 | 0.003352627 | 0.046140796 | NR4A3, IRF4, SHOX2, TNNI2, MDFIC, |
| | | regulation of | | | | | | | HOXA5, NFATC2, SATB2, NPAS2, |
| | | RNA biosynthetic | | | | | | | NR4A1, GDF6, DLX5, CDH13, HIVEP3, |
| | | process | | | | | | | BMP2, WNT4, IL6, TBX5, NAMPT, |
| | | | | | | | | | FOXO1 |
| upregulated | GO: 2000147 | positive | 139 | 15462 | 274 | 8 | 0.003353275 | 0.046140796 | CDH13, SEMA5A, CCR1, BMP2, PTPRC, |
| | | regulation of cell | | | | | | | ITGB3, ADRA2A, IL6 |
| | | motility | | | | | | | |
| upregulated | GO: 0035556 | intracellular | 139 | 15462 | 2195 | 32 | 0.003365295 | 0.046140796 | TAGAP, CBLC, DCLK1, SEMA5A, SOS1, |
| | | signal | | | | | | | ADRA2A, PLA2R1, MDFIC, RAB30, |
| | | transduction | | | | | | | CD36, MCTP1, DOCK8, LRRK2, BIRC3, |
| | | | | | | | | | NKD1, NR4A1, IL18R1, CDH13, |
| | | | | | | | | | RASGRF2, DEPDC7, GUCY1A3, |
| | | | | | | | | | COL1A2, CCR1, FYB, CAMK2D, BMP2, |
| | | | | | | | | | TNFRSF11A, WNT4, IL6, CXCL9, |
| | | | | | | | | | ADIPOQ, FOXO1 |
| upregulated | GO: 0045597 | positive | 139 | 15462 | 539 | 12 | 0.003427628 | 0.046140796 | GDF6, DLX5, SEMA5A, SHOX2, CCR1, |
| | | regulation of cell | | | | | | | BMP2, RGCC, WNT4, IL6, HOXA5, CD36, |
| | | differentiation | | | | | | | ADIPOQ |
| upregulated | GO: 0032341 | aldosterone | 139 | 15462 | 10 | 2 | 0.003444031 | 0.046140796 | WNT4, BMP2 |
| | | metabolic | | | | | | | |
| | | process | | | | | | | |
| upregulated | GO: 0003181 | atrioventricular | 139 | 15462 | 10 | 2 | 0.003444031 | 0.046140796 | TBX5, BMP2 |
| | | valve | | | | | | | |
| | | morphogenesis | | | | | | | |
| upregulated | GO: 0072539 | T-helper 17 cell | 139 | 15462 | 10 | 2 | 0.003444031 | 0.046140796 | IL6, IRF4 |
| | | differentiation | | | | | | | |
| upregulated | GO: 0002295 | T-helper cell | 139 | 15462 | 10 | 2 | 0.003444031 | 0.046140796 | IL6, IRF4 |
| | | lineage | | | | | | | |
| | | commitment | | | | | | | |
| upregulated | GO: 0043373 | CD4-positive, | 139 | 15462 | 10 | 2 | 0.003444031 | 0.046140796 | IL6, IRF4 |
| | | alpha-beta T cell | | | | | | | |
| | | lineage | | | | | | | |
| | | commitment | | | | | | | |
| upregulated | GO: 0051050 | positive | 139 | 15462 | 541 | 12 | 0.003530947 | 0.047094981 | IL18R1, SEMA5A, CCR1, RGCC, |
| | | regulation of | | | | | | | TNFRSF11A, CD2, PLA2R1, IL6, CXCL9, |
| | | transport | | | | | | | CD36, CNTN1, ADIPOQ |
| upregulated | GO: 0045937 | positive | 139 | 15462 | 840 | 16 | 0.003608223 | 0.047701662 | FAM129A, ADRA2A, MDFIC, CD36, |
| | | regulation of | | | | | | | LRRK2, GDF6, PTPRC, BMP2, |
| | | phosphate | | | | | | | GUCY1A3, CCR1, ITGB3, TNFRSF11A, |
| | | metabolic | | | | | | | RGCC, IL6, CXCL9, ADIPOQ |
| | | process | | | | | | | |
| upregulated | GO: 0010562 | positive | 139 | 15462 | 840 | 16 | 0.003608223 | 0.047701662 | FAM129A, ADRA2A, MDFIC, CD36, |
| | | regulation of | | | | | | | LRRK2, GDF6, PTPRC, BMP2, |
| | | phosphorus | | | | | | | GUCY1A3, CCR1, ITGB3, TNFRSF11A, |
| | | metabolic | | | | | | | RGCC, IL6, CXCL9, ADIPOQ |
| | | process | | | | | | | |
| upregulated | GO: 0007399 | nervous system | 139 | 15462 | 1848 | 28 | 0.003701949 | 0.048726093 | NR4A3, SHOX2, TENM3, DCLK1, SOS1, |
| | | development | | | | | | | SEMA5A, PDLIM5, LSAMP, SATB2, |
| | | | | | | | | | LRRK2, NPAS2, NKD1, GDF6, PRKG1, |
| | | | | | | | | | NPTX1, DLX5, GFRA1, SDC2, BMP2, |
| | | | | | | | | | PTPRC, ITGB3, WNT4, PRDM6, IL6, |
| | | | | | | | | | ASIC2, SLC7A5, BCHE, CNTN1 |
| upregulated | GO: 0002294 | CD4-positive, | 139 | 15462 | 35 | 3 | 0.003770349 | 0.048774172 | IL6, IL18R1, IRF4 |
| | | alpha-beta T cell | | | | | | | |
| | | differentiation | | | | | | | |
| | | involved in | | | | | | | |
| | | immune | | | | | | | |
| | | response | | | | | | | |
| upregulated | GO: 0042093 | T-helper cell | 139 | 15462 | 35 | 3 | 0.003770349 | 0.048774172 | IL6, IL18R1, IRF4 |
| | | differentiation | | | | | | | |
| upregulated | GO: 0090132 | epithelium | 139 | 15462 | 165 | 6 | 0.003770612 | 0.048774172 | RGCC, ITGB3. NR4A1, CDH13, DPP4, |
| | | migration | | | | | | | SEMA5A |
| upregulated | GO: 0010631 | epithelial cell | 139 | 15462 | 165 | 6 | 0.003770612 | 0.048774172 | RGCC, ITGB3. NR4A1, CDH13, DPP4, |
| | | migration | | | | | | | SEMA5A |
| upregulated | GO: 1901184 | regulation of | 139 | 15462 | 71 | 4 | 0.003814578 | 0.048808552 | ADRA2A, CBLC, CDH13, SOS1 |
| | | ERBB signaling | | | | | | | |
| | | pathway | | | | | | | |
| upregulated | GO: 0006979 | response to | 139 | 15462 | 280 | 8 | 0.003822063 | 0.048808552 | NR4A3, LRRK2, IL6, PLA2R1, ERCC1, |
| | | oxidative stress | | | | | | | CD36, FOXO1, ADIPOQ |
| upregulated | GO: 0051272 | positive | 139 | 15462 | 280 | 8 | 0.003822063 | 0.048808552 | CDH13, SEMA5A, CCR1, BMP2, PTPRC, |
| | | regulation of | | | | | | | ITGB3, ADRA2A, IL6 |
| | | cellular | | | | | | | |
| | | component | | | | | | | |
| | | movement | | | | | | | |
| upregulated | GO: 0023014 | signal | 139 | 15462 | 619 | 13 | 0.003845017 | 0.048893623 | LRRK2, NKD1, CBLC, CCR1, BMP2, |
| | | transduction by | | | | | | | TNFRSF11A, ADRA2A, WNT4, IL6, |
| | | phosphorylation | | | | | | | MDFIC, CD36, ADIPOQ, FOXO1 |
| upregulated | GO: 0022411 | cellular | 139 | 15462 | 410 | 10 | 0.003944418 | 0.049945981 | ELN, DPP4, SEMA5A, ADAMTS5, |
| | | component | | | | | | | COL1A2, COL12A1, TBX5, CD36, MMP1, |
| | | disassembly | | | | | | | ADIPOQ |
| upregulated | GO: 0070206 | protein | 139 | 15462 | 36 | 3 | 0.004086319 | 0.051167232 | ALOX5AP, COL1A2, ADIPOQ |
| | | trimerization | | | | | | | |
| upregulated | GO: 0045599 | negative | 139 | 15462 | 36 | 3 | 0.004086319 | 0.051167232 | IL6, ADIPOQ, FOXO1 |
| | | regulation of fat | | | | | | | |
| | | cell | | | | | | | |
| | | differentiation | | | | | | | |
| upregulated | GO: 0051224 | negative | 139 | 15462 | 117 | 5 | 0.004094233 | 0.051167232 | RGCC, IL6, MDFIC, CD36, ADIPOQ |
| | | regulation of | | | | | | | |
| | | protein transport | | | | | | | |
| upregulated | GO: 0051254 | positive | 139 | 15462 | 1173 | 20 | 0.004109065 | 0.051167232 | NR4A3, IRF4, SHOX2, TNNI2, MDFIC, |
| | | regulation of | | | | | | | HOXA5, NFATC2, SATB2, NPAS2, |
| | | RNA metabolic | | | | | | | NR4A1, GDF6, DLX5, CDH13, HIVEP3, |
| | | process | | | | | | | BMP2, WNT4, IL6, TBX5, NAMPT, |
| | | | | | | | | | FOXO1 |
| upregulated | GO: 0070208 | protein | 139 | 15462 | 11 | 2 | 0.004184643 | 0.051440901 | ADIPOQ, COL1A2 |
| | | heterotrimerization | | | | | | | |
| upregulated | GO: 0002363 | alpha-beta T cell | 139 | 15462 | 11 | 2 | 0.004184643 | 0.051440901 | IL6, IRF4 |
| | | lineage | | | | | | | |
| | | commitment | | | | | | | |
| upregulated | GO: 0045596 | negative | 139 | 15462 | 482 | 11 | 0.004186018 | 0.051440901 | SEMA5A, BMP2, ITGB3, EFEMP1, WNT4, |
| | | regulation of cell | | | | | | | PRDM6, IL6, HOXA5, TBX5, ADIPOQ, |
| | | differentiation | | | | | | | FOXO1 |
| upregulated | GO: 0032989 | cellular | 139 | 15462 | 1012 | 18 | 0.004199607 | 0.051440901 | NR4A3, DCLK1, SOS1, SEMA5A, |
| | | component | | | | | | | SHOX2, PDLIM5, LRRK2, GFRA1, DLX5, |
| | | morphogenesis | | | | | | | NPTX1, SDC2, PTPRC, BMP2, RGCC, |
| | | | | | | | | | ITGB3, WNT4, TBX5, CNTN1 |
| upregulated | GO: 0043270 | positive | 139 | 15462 | 118 | 5 | 0.004245107 | 0.051786854 | TNFRSF11A, PLA2R1, CXCL9, CCR1, |
| | | regulation of ion | | | | | | | CNTN1 |
| | | transport | | | | | | | |
| upregulated | GO: 0048646 | anatomical | 139 | 15462 | 855 | 16 | 0.004280852 | 0.05201148 | NR4A3, SEMA5A, HOXA5, THSD7A, |
| | | structure | | | | | | | NKD1, NR4A1, DLX5, CDH13, ANGPTL4, |
| | | formation | | | | | | | BMP2, ITGB3, RGCC, WNT4, IL6, TBX5, |
| | | involved in | | | | | | | ERCC1 |
| | | morphogenesis | | | | | | | |
| upregulated | GO: 0090130 | tissue migration | 139 | 15462 | 170 | 6 | 0.0043614 | 0.052152179 | RGCC, ITGB3. NR4A1, CDH13, DPP4, |
| | | | | | | | | | SEMA5A |
| upregulated | GO: 0002287 | alpha-beta T cell | 139 | 15462 | 37 | 3 | 0.004417926 | 0.052152179 | IL6, IL18R1, IRF4 |
| | | activation | | | | | | | |
| | | involved in | | | | | | | |
| | | immune | | | | | | | |
| | | response | | | | | | | |
| upregulated | GO: 0002293 | alpha-beta T cell | 139 | 15462 | 37 | 3 | 0.004417926 | 0.052152179 | IL6, IL18R1, IRF4 |
| | | differentiation | | | | | | | |
| | | involved in | | | | | | | |
| | | immune | | | | | | | |
| | | response | | | | | | | |
| upregulated | GO: 0044246 | regulation of | 139 | 15462 | 37 | 3 | 0.004417926 | 0.052152179 | RGCC, WNT4, IL6 |
| | | multicellular | | | | | | | |
| | | organismal | | | | | | | |
| | | metabolic | | | | | | | |
| | | process | | | | | | | |
| upregulated | GO: 0048660 | regulation of | 139 | 15462 | 74 | 4 | 0.004424895 | 0.052152179 | IL6, CDH13, PDE1A, ADIPOQ |
| | | smooth muscle | | | | | | | |
| | | cell proliferation | | | | | | | |
| upregulated | GO: 0032368 | regulation of lipid | 139 | 15462 | 74 | 4 | 0.004424895 | 0.052152179 | TNFRSF11A, ITGB3, PLA2R1, ADIPOQ |
| | | transport | | | | | | | |
| upregulated | GO: 0050729 | positive | 139 | 15462 | 74 | 4 | 0.004424895 | 0.052152179 | TNFRSF11A, IL6, ALOX5AP, FABP4 |
| | | regulation of | | | | | | | |
| | | inflammatory | | | | | | | |
| | | response | | | | | | | |
| upregulated | GO: 0042326 | negative | 139 | 15462 | 287 | 8 | 0.004431458 | 0.052152179 | FAM129A, BIRC3, CBLC, FABP4, |
| | | regulation of | | | | | | | PTPRC, IL6, FOXO1, ADIPOQ |
| | | phosphorylation | | | | | | | |
| upregulated | GO: 0030217 | T cell | 139 | 15462 | 171 | 6 | 0.004487318 | 0.052603285 | CD2, WNT4, IL6, IL18R1, IRF4, PTPRC |
| | | differentiation | | | | | | | |
| upregulated | GO: 0040017 | positive | 139 | 15462 | 289 | 8 | 0.004618619 | 0.053931815 | CDH13, SEMA5A, CCR1, BMP2, PTPRC, |
| | | regulation of | | | | | | | ITGB3, ADRA2A, IL6 |
| | | locomotion | | | | | | | |
| upregulated | GO: 0001934 | positive | 139 | 15462 | 635 | 13 | 0.004758046 | 0.055215982 | FAM129A, LRRK2, GDF6, BMP2, PTPRC, |
| | | regulation of | | | | | | | RGCC, TNFRSF11A, ITGB3, ADRA2A, |
| | | protein | | | | | | | IL6, MDFIC, CD36, ADIPOQ |
| | | phosphorylation | | | | | | | |
| upregulated | GO: 0010883 | regulation of lipid | 139 | 15462 | 38 | 3 | 0.004765391 | 0.055215982 | ITGB3, IL6, CD36 |
| | | storage | | | | | | | |
| upregulated | GO: 0030030 | cell projection | 139 | 15462 | 1026 | 18 | 0.004841049 | 0.055683941 | NR4A3, SHOX2, TENM3, DCLK1, SOS1, |
| | | organization | | | | | | | SEMA5A, PDLIM5, LRRK2, PRKG1, |
| | | | | | | | | | NPTX1, GFRA1, DLX5, SDC2, CDH13, |
| | | | | | | | | | PTPRC, ITGB3, IL6, CNTN1 |
| upregulated | GO: 0051240 | positive | 139 | 15462 | 563 | 12 | 0.004842889 | 0.055683941 | BIRC3, IL18R1, CAMK2D, BMP2, RGCC, |
| | | regulation of | | | | | | | TNFRSF11A, CD2, ADRA2A, WNT4, IL6, |
| | | multicellular | | | | | | | CD36, ADIPOQ |
| | | organismal | | | | | | | |
| | | process | | | | | | | |
| upregulated | GO: 0016202 | regulation of | 139 | 15462 | 76 | 4 | 0.004866405 | 0.055692445 | TBX5, MEGF10, SHOX2, BMP2 |
| | | striated muscle | | | | | | | |
| | | tissue | | | | | | | |
| | | development | | | | | | | |
| upregulated | GO: 0002684 | positive | 139 | 15462 | 638 | 13 | 0.004947293 | 0.055692445 | BIRC3, TLR1, IRF4, DPP4, CCR1, FYB, |
| | | regulation of | | | | | | | PTPRC, RGCC, CD2, LCP2, IL6, |
| | | immune system | | | | | | | NFATC2, CD36 |
| | | process | | | | | | | |
| upregulated | GO: 0032305 | positive | 139 | 15462 | 12 | 2 | 0.004992092 | 0.055692445 | TNFRSF11A, PLA2R1 |
| | | regulation of | | | | | | | |
| | | icosanoid | | | | | | | |
| | | secretion | | | | | | | |
| upregulated | GO: 0006705 | mineralocorticoid | 139 | 15462 | 12 | 2 | 0.004992092 | 0.055692445 | WNT4, BMP2 |
| | | biosynthetic | | | | | | | |
| | | process | | | | | | | |
| upregulated | GO: 0043369 | CD4-positive or | 139 | 15462 | 12 | 2 | 0.004992092 | 0.055692445 | IL6, IRF4 |
| | | CD8-positive, | | | | | | | |
| | | alpha-beta T cell | | | | | | | |
| | | lineage | | | | | | | |
| | | commitment | | | | | | | |
| upregulated | GO: 1902931 | negative | 139 | 15462 | 12 | 2 | 0.004992092 | 0.055692445 | WNT4, BMP2 |
| | | regulation of | | | | | | | |
| | | alcohol | | | | | | | |
| | | biosynthetic | | | | | | | |
| | | process | | | | | | | |
| upregulated | GO: 0032799 | low-density | 139 | 15462 | 12 | 2 | 0.004992092 | 0.055692445 | ITGB3, ADIPOQ |
| | | lipoprotein | | | | | | | |
| | | receptor particle | | | | | | | |
| | | metabolic | | | | | | | |
| | | process | | | | | | | |
| upregulated | GO: 0072538 | T-helper 17 type | 139 | 15462 | 12 | 2 | 0.004992092 | 0.055692445 | IL6, IRF4 |
| | | immune | | | | | | | |
| | | response | | | | | | | |
| upregulated | GO: 1901861 | regulation of | 139 | 15462 | 77 | 4 | 0.005097861 | 0.055970574 | TBX5, MEGF10, SHOX2, BMP2 |
| | | muscle tissue | | | | | | | |
| | | development | | | | | | | |
| upregulated | GO: 0032680 | regulation of | 139 | 15462 | 77 | 4 | 0.005097861 | 0.055970574 | CD2, TLR1, CD36, ADIPOQ |
| | | tumor necrosis | | | | | | | |
| | | factor production | | | | | | | |
| upregulated | GO: 0048659 | smooth muscle | 139 | 15462 | 77 | 4 | 0.005097861 | 0.055970574 | IL6, CDH13, PDE1A, ADIPOQ |
| | | cell proliferation | | | | | | | |
| upregulated | GO: 0032640 | tumor necrosis | 139 | 15462 | 77 | 4 | 0.005097861 | 0.055970574 | CD2, TLR1, CD36, ADIPOQ |
| | | factor production | | | | | | | |
| upregulated | GO: 0048858 | cell projection | 139 | 15462 | 716 | 14 | 0.005119761 | 0.055970574 | NR4A3, LRRK2, NPTX1, DLX5, GFRA1, |
| | | morphogenesis | | | | | | | SDC2, DCLK1, SOS1, SEMA5A, SHOX2, |
| | | | | | | | | | PTPRC, ITGB3, PDLIM5, CNTN1 |
| upregulated | GO: 0002292 | Tcell | | | | | | | |
| | | differentiation | 139 | 15462 | 39 | 3 | 0.005128926 | 0.055970574 | IL6, IL18R1, IRF4 |
| | | involved in | | | | | | | |
| | | immune | | | | | | | |
| | | response | | | | | | | |
| upregulated | GO: 0061138 | morphogenesis | 139 | 15462 | 176 | 6 | 0.005157543 | 0.056078932 | WNT4, IL6, HOXA5, FEM1B, SEMA5A, |
| | | of a branching | | | | | | | BMP2 |
| | | epithelium | | | | | | | |
| upregulated | GO: 0002521 | leukocyte | 139 | 15462 | 361 | 9 | 0.005356257 | 0.057947221 | IL18R1, IRF4, CCR1, PTPRC, |
| | | differentiation | | | | | | | TNFRSF11A, CD2, WNT4, IL6, ADIPOQ |
| upregulated | GO: 0016337 | single | 139 | 15462 | 235 | 7 | 0.005367986 | 0.057947221 | RGCC, CD2, WNT4, MEGF10, DPP4, |
| | | organismal cell- | | | | | | | ADIPOQ, BMP2 |
| | | cell adhesion | | | | | | | |
| upregulated | GO: 0030155 | regulation of cell | 139 | 15462 | 298 | 8 | 0.005537253 | 0.05947035 | RGCC, WNT4, CDH13, DPP4, SEMA5A, |
| | | adhesion | | | | | | | CD36, ADIPOQ, BMP2 |
| upregulated | GO: 0070201 | regulation of | 139 | 15462 | 363 | 9 | 0.005548716 | 0.05947035 | TLR1, IL18R1, SEMA5A, CD2, RGCC, |
| | | establishment of | | | | | | | IL6, MDFIC, CD36, ADIPOQ |
| | | protein | | | | | | | |
| | | localization | | | | | | | |
| upregulated | GO: 0045598 | regulation of fat | 139 | 15462 | 79 | 4 | 0.005582683 | 0.059621465 | IL6, ADIPOQ, BMP2, FOXO1 |
| | | cell | | | | | | | |
| | | differentiation | | | | | | | |
| upregulated | GO: 0033157 | regulation of | 139 | 15462 | 180 | 6 | 0.005744484 | 0.06089662 | IL6, MDFIC, IL18R1, SEMA5A, CD36, |
| | | intracellular | | | | | | | ADIPOQ |
| | | protein transport | | | | | | | |
| upregulated | GO: 0061448 | connective tissue | 139 | 15462 | 180 | 6 | 0.005744484 | 0.06089662 | EFEMP1, HOXA5, NAMPT, SHOX2, |
| | | development | | | | | | | SATB2, BMP2 |
| upregulated | GO: 0090030 | regulation of | 139 | 15462 | 13 | 2 | 0.005865135 | 0.06089662 | WNT4, BMP2 |
| | | steroid hormon | | | | | | | |
| | | biosynthetic | | | | | | | |
| | | process | | | | | | | |
| upregulated | GO: 0045986 | negative | 139 | 15462 | 13 | 2 | 0.005865135 | 0.06089662 | ADRA2A, GUCY1A3 |
| | | regulation of | | | | | | | |
| | | smooth muscle | | | | | | | |
| | | contraction | | | | | | | |
| upregulated | GO: 0006700 | C21-steroid | 139 | 15462 | 13 | 2 | 0.005865135 | 0.06089662 | WNT4, BMP2 |
| | | hormone | | | | | | | |
| | | biosynthetic | | | | | | | |
| | | process | | | | | | | |
| upregulated | GO: 0010745 | negative | 139 | 15462 | 13 | 2 | 0.005865135 | 0.06089662 | ITGB3, ADIPOQ |
| | | regulation of | | | | | | | |
| | | macrophage | | | | | | | |
| | | derived foam cell | | | | | | | |
| | | differentiation | | | | | | | |
| upregulated | GO: 0008212 | mineralocorticoid | 139 | 15462 | 13 | 2 | 0.005865135 | 0.06089662 | WNT4, BMP2 |
| | | metabolic | | | | | | | |
| | | process | | | | | | | |
| upregulated | GO: 0050730 | regulation of | 139 | 15462 | 181 | 6 | 0.005898547 | 0.06089662 | ADRA2A, ITGB3, IL6, CBLC, CD36, |
| | | peptidyl-tyrosine | | | | | | | ADIPOQ |
| | | phosphorylation | | | | | | | |
| upregulated | GO: 0001763 | morphogenesis | 139 | 15462 | 181 | 6 | 0.005898547 | 0.06089662 | WNT4, IL6, HOXA5, FEM1B, SEMA5A, |
| | | of a branching | | | | | | | BMP2 |
| | | structure | | | | | | | |
| upregulated | GO: 0032370 | positive | 139 | 15462 | 41 | 3 | 0.005905004 | 0.06089662 | TNFRSF11A, PLA2R1, ADIPOQ |
| | | regulation of lipid | | | | | | | |
| | | transport | | | | | | | |
| upregulated | GO: 0010033 | response to | 139 | 15462 | 2187 | 31 | 0.005989336 | 0.061554781 | PAPPA, NR4A3, TLR1, IRF4, SOS1, |
| | | organic | | | | | | | ADRA2A, FOSL2, CD36, CALB1, SATB2, |
| | | substance | | | | | | | LRRK2, NR4A1, NPTX1, DLX5, IL18R1, |
| | | | | | | | | | PDE1A, CDH13, RASGRF2, PTPRC, |
| | | | | | | | | | CAMK2D, BMP2, CCR1, COL1A2, |
| | | | | | | | | | TNFRSF11A, ITGB3, WNT4, IL6, NAMPT, |
| | | | | | | | | | BCHE, ADIPOQ, FOXO1 |
| upregulated | GO: 0010563 | negative | 139 | 15462 | 368 | 9 | 0.006053359 | 0.061789554 | FAM129A, BIRC3, CBLC, FABP4, |
| | | regulation of | | | | | | | PTPRC, ADRA2A, IL6, FOXO1, ADIPOQ |
| | | phosphorus | | | | | | | |
| | | metabolic | | | | | | | |
| | | process | | | | | | | |
| upregulated | GO: 0045936 | negative | 139 | 15462 | 368 | 9 | 0.006053359 | 0.061789554 | FAM129A, BIRC3, CBLC, FABP4, |
| | | regulation of | | | | | | | PTPRC, ADRA2A, IL6, FOXO1, ADIPOQ |
| | | phosphate | | | | | | | |
| | | metabolic | | | | | | | |
| | | process | | | | | | | |
| upregulated | GO: 0071706 | tumor necrosis | 139 | 15462 | 81 | 4 | 0.006097382 | 0.061801978 | CD2, TLR1, CD36, ADIPOQ |
| | | factor | | | | | | | |
| | | superfamily | | | | | | | |
| | | cytokine | | | | | | | |
| | | production | | | | | | | |
| upregulated | GO: 0032990 | cell part | 139 | 15462 | 731 | 14 | 0.006119827 | 0.061801978 | NR4A3, LRRK2, NPTX1, DLX5, GFRA1, |
| | | morphogenesis | | | | | | | SDC2, DCLK1, SOS1, SEMA5A, SHOX2, |
| | | | | | | | | | PTPRC, ITGB3, PDLIM5, CNTN1 |
| upregulated | GO: 0072359 | circulatory | 139 | 15462 | 809 | 15 | 0.006136951 | 0.061801978 | NR4A1, CDH13, ANGPTL4, SEMA5A, |
| | | system | | | | | | | SHOX2, BMP2, COL1A2, RGCC, ITGB3, |
| | | development | | | | | | | WNT4, IL6, HOXA5, THSD7A, TBX5, |
| | | | | | | | | | FOXO1 |
| upregulated | GO: 0072358 | cardiovascular | 139 | 15462 | 809 | 15 | 0.006136951 | 0.061801978 | NR4A1, CDH13, ANGPTL4, SEMA5A, |
| | | system | | | | | | | SHOX2, BMP2, COL1A2, RGCC, ITGB3, |
| | | development | | | | | | | WNT4, IL6, HOXA5, THSD7A, TBX5, |
| | | | | | | | | | FOXO1 |
| upregulated | GO: 0042981 | regulation of | 139 | 15462 | 1217 | 20 | 0.006162322 | 0.061849932 | NR4A3, SOS1, SEMA5A, HOXA5, |
| | | apoptotic | | | | | | | FEM1B, BNIP3L, BIRC3, NR4A1, PDE1A, |
| | | process | | | | | | | RASGRF2, ANGPTL4, PNMA2, BMP2, |
| | | | | | | | | | RGCC, WNT4, IL6, TBX5, ASIC2, FOXO1, |
| | | | | | | | | | ADIPOQ |
| upregulated | GO: 0043367 | CD4-positive, | 139 | 15462 | 42 | 3 | 0.006317922 | 0.063200282 | IL6, IL18R1, IRF4 |
| | | alpha-beta T cell | | | | | | | |
| | | differentiation | | | | | | | |
| upregulated | GO: 0007409 | axonogenesis | 139 | 15462 | 511 | 11 | 0.006433084 | 0.06413849 | NR4A3, NPTX1, DLX5, GFRA1, DCLK1, |
| | | | | | | | | | SEMA5A, SHOX2, SOS1, PTPRC, ITGB3, |
| | | | | | | | | | CNTN1 |
| upregulated | GO: 0051960 | regulation of | 139 | 15462 | 512 | 11 | 0.006524777 | 0.064837272 | LRRK2, GDF6, SDC2, TENM3, SEMA5A, |
| | | nervous system | | | | | | | SHOX2, BMP2, IL6, ASIC2, PDLIM5, |
| | | development | | | | | | | CNTN1 |
| upregulated | GO: 0071902 | positive | 139 | 15462 | 244 | 7 | 0.006558312 | 0.064955423 | RGCC, ADRA2A, TNFRSF11A, LRRK2, |
| | | regulation of | | | | | | | MDFIC, ADIPOQ, BMP2 |
| | | protein | | | | | | | |
| | | serine/threonine | | | | | | | |
| | | kinase activity | | | | | | | |
| upregulated | GO: 0043408 | regulation of | 139 | 15462 | 513 | 11 | 0.006617496 | 0.065146536 | LRRK2, CBLC, CCR1, BMP2, |
| | | MAPK cascade | | | | | | | TNFRSF11A, ADRA2A, MDFIC, IL6, |
| | | | | | | | | | CD36, ADIPOQ, FOXO1 |
| upregulated | GO: 0045944 | positive | 139 | 15462 | 816 | 15 | 0.006627218 | 0.065146536 | NR4A3, NPAS2, NR4A1, DLX5, CDH13, |
| | | regulation of | | | | | | | IRF4, SHOX2, BMP2, IL6, HOXA5, TBX5, |
| | | transcription | | | | | | | NAMPT, NFATC2, SATB2, FOXO1 |
| | | from RNA | | | | | | | |
| | | polymerase II | | | | | | | |
| | | promoter | | | | | | | |
| upregulated | GO: 0030177 | positive | 139 | 15462 | 83 | 4 | 0.006642732 | 0.065146536 | WNT4, NKD1, DLX5, BMP2 |
| | | regulation of Wnt | | | | | | | |
| | | signaling | | | | | | | |
| | | pathway | | | | | | | |
| upregulated | GO: 0050790 | regulation of | 139 | 15462 | 2020 | 29 | 0.006694661 | 0.065210999 | TAGAP, CBLC, SOS1, FABP4, ADRA2A, |
| | | catalytic activity | | | | | | | MDFIC, PLA2R1, PPP1R3B, FEM1B, |
| | | | | | | | | | ALOX5AP, DOCK8, LRRK2, BIRC3, |
| | | | | | | | | | NR4A1, PRKG1, RASGRF2, PDE1A, |
| | | | | | | | | | ANGPTL4, BMP2, PTPRC, CAMK2D, |
| | | | | | | | | | TNFRSF11A, ITGB3, RGCC, WNT4, IL6, |
| | | | | | | | | | TFPI2, ADIPOQ, ARHGAP42 |
| upregulated | GO: 0002252 | immune effector | 139 | 15462 | 514 | 11 | 0.006711248 | 0.065210999 | SLAMF7, BIRC3, IL18R1, IRF4, PTPRC, |
| | | process | | | | | | | RGCC, IL6, ERCC1, BNIP3L, CXCL9, |
| | | | | | | | | | CD36 |
| upregulated | GO: 0002250 | adaptive immune | 139 | 15462 | 186 | 6 | 0.006714495 | 0.065210999 | TNFRSF11A, IL6, ERCC1, IGJ, IL18R1, |
| | | response | | | | | | | IRF4 |
| upregulated | GO: 2000193 | positive | 139 | 15462 | 14 | 2 | 0.006802544 | 0.065641265 | TNFRSF11A, PLA2R1 |
| | | regulation of fatty | | | | | | | |
| | | acid transport | | | | | | | |
| upregulated | GO: 0060192 | negative | 139 | 15462 | 14 | 2 | 0.006802544 | 0.065641265 | PLA2R1, ANGPTL4 |
| | | regulation of | | | | | | | |
| | | lipase activity | | | | | | | |
| upregulated | GO: 0042327 | positive | 139 | 15462 | 741 | 14 | 0.006869897 | 0.066078722 | FAM129A, LRRK2, GDF6, CCR1, BMP2, |
| | | regulation of | | | | | | | PTPRC, RGCC, TNFRSF11A, ITGB3, |
| | | phosphorylation | | | | | | | ADRA2A, IL6, MDFIC, CD36, ADIPOQ |
| upregulated | GO: 0071241 | cellular response | 139 | 15462 | 84 | 4 | 0.006927139 | 0.066416437 | ALOX5AP, FOXO1, FABP4, CAMK2D |
| | | to inorganic | | | | | | | |
| | | substance | | | | | | | |
| upregulated | GO: 0050731 | positive | 139 | 15462 | 133 | 5 | 0.007010556 | 0.067002163 | ADRA2A, ITGB3, IL6, CD36, ADIPOQ |
| | | regulation of | | | | | | | |
| | | peptidyl-tyrosine | | | | | | | |
| | | phosphorylation | | | | | | | |
| upregulated | GO: 0051094 | positive | 139 | 15462 | 744 | 14 | 0.007108753 | 0.067724979 | GDF6, DLX5, ANGPTL4, SEMA5A, |
| | | regulation of | | | | | | | SHOX2, CCR1, BMP2, RGCC, WNT4, |
| | | developmental | | | | | | | IL6, HOXA5, ASIC2, CD36, ADIPOQ |
| | | process | | | | | | | |
| upregulated | GO: 0001525 | angiogenesis | 139 | 15462 | 378 | 9 | 0.007168498 | 0.068078046 | NR4A1, CDH13, SEMA5A, ANGPTL4, |
| | | | | | | | | | ITGB3, RGCC, IL6, HOXA5, THSD7A |
| upregulated | GO: 0032879 | regulation of | 139 | 15462 | 1670 | 25 | 0.007220919 | 0.068341281 | TLR1, SEMA5A, ADRA2A, CD2, MDFIC, |
| | | localization | | | | | | | PLA2R1, CD36, LRRK2, NKD1, CDH13, |
| | | | | | | | | | IL18R1, CCR1, CAMK2D, PTPRC, BMP2, |
| | | | | | | | | | TNFRSF11A, RGCC, ITGB3, WNT4, IL6, |
| | | | | | | | | | TBX5, ASIC2, CXCL9, CNTN1, ADIPOQ |
| upregulated | GO: 0001649 | osteoblast | 139 | 15462 | 189 | 6 | 0.007241762 | 0.068341281 | WNT4, IL6, DLX5, SHOX2, SATB2, BMP2 |
| | | differentiation | | | | | | | |
| upregulated | GO: 0050921 | positive | 139 | 15462 | 86 | 4 | 0.007519877 | 0.070743417 | IL6, CDH13, SEMA5A, CCR1 |
| | | regulation of | | | | | | | |
| | | chemotaxis | | | | | | | |
| upregulated | GO: 1901342 | regulation of | 139 | 15462 | 191 | 6 | 0.007609531 | 0.071166555 | RGCC, WNT4, IL6, HOXA5, ANGPTL4, |
| | | vasculature | | | | | | | SEMA5A |
| | | development | | | | | | | |
| upregulated | GO: 0070482 | response to | 139 | 15462 | 251 | 7 | 0.007612284 | 0.071166555 | RGCC, SDC2, ANGPTL4, DPP4, |
| | | oxygen levels | | | | | | | ADIPOQ, BMP2, FOXO1 |
| upregulated | GO: 0002253 | activation of | 139 | 15462 | 382 | 9 | 0.007656253 | 0.071182939 | RGCC, LCP2, BIRC3, TLR1, NFATC2, |
| | | immune | | | | | | | IRF4, CD36, PTPRC, FYB |
| | | response | | | | | | | |
| upregulated | GO: 0035767 | endothelial cell | 139 | 15462 | 15 | 2 | 0.007803108 | 0.071182939 | NR4A1, SEMA5A |
| | | Chemotaxis | | | | | | | |
| upregulated | GO: 0072202 | cell | 139 | 15462 | 15 | 2 | 0.007803108 | 0.071182939 | WNT4, ADIPOQ |
| | | differentiation | | | | | | | |
| | | involved in | | | | | | | |
| | | metanephros | | | | | | | |
| | | development | | | | | | | |
| upregulated | GO: 1903053 | regulation of | 139 | 15462 | 15 | 2 | 0.007803108 | 0.071182939 | RGCC, DPP4 |
| | | extracellular | | | | | | | |
| | | matrix | | | | | | | |
| | | organization | | | | | | | |
| upregulated | GO: 0032303 | regulation of | 139 | 15462 | 15 | 2 | 0.007803108 | 0.071182939 | TNFRSF11A, PLA2R1 |
| | | icosanoid | | | | | | | |
| | | secretion | | | | | | | |
| upregulated | GO: 0002070 | epithelial cell | 139 | 15462 | 15 | 2 | 0.007803108 | 0.071182939 | FEM1B, HOXA5 |
| | | maturation | | | | | | | |
| upregulated | GO: 0061564 | axon | 139 | 15462 | 525 | 11 | 0.007812989 | 0.071182939 | NR4A3, NPTX1, DLX5, GFRA1, DCLK1, |
| | | development | | | | | | | SOS1, SEMA5A, SHOX2, PTPRC, ITGB3, |
| | | | | | | | | | CNTN1 |
| upregulated | GO: 0051216 | cartilage | 139 | 15462 | 137 | 5 | 0.00792075 | 0.071182939 | EFEMP1, HOXA5, SHOX2, SATB2, BMP2 |
| | | development | | | | | | | |
| upregulated | GO: 0051049 | regulation of | 139 | 15462 | 1248 | 20 | 0.008067085 | 0.071182939 | TLR1, SEMA5A, ADRA2A, CD2, MDFIC, |
| | | transport | | | | | | | PLA2R1, CD36, LRRK2, CDH13, IL18R1, |
| | | | | | | | | | CCR1, CAMK2D, TNFRSF11A, RGCC, |
| | | | | | | | | | ITGB3, IL6, ASIC2, CXCL9, ADIPOQ, |
| | | | | | | | | | CNTN1 |
| upregulated | GO: 0035710 | CD4-positive, | 139 | 15462 | 46 | 3 | 0.008139429 | 0.071182939 | IL6, IL18R1, IRF4 |
| | | alpha-beta T cell | | | | | | | |
| | | activation | | | | | | | |
| upregulated | GO: 0048813 | dendrite | 139 | 15462 | 88 | 4 | 0.008145111 | 0.071182939 | LRRK2, PDLIM5, SDC2, DCLK1 |
| | | morphogenesis | | | | | | | |
| upregulated | GO: 0001837 | epithelial to | 139 | 15462 | 88 | 4 | 0.008145111 | 0.071182939 | RGCC, WNT4, TBX5, BMP2 |
| | | mesenchymal | | | | | | | |
| | | transition | | | | | | | |
| upregulated | GO: 0009306 | protein secretion | 139 | 15462 | 194 | 6 | 0.008186227 | 0.071182939 | RGCC, CD2, LCP2, IL6, TLR1, CBLN4 |
| upregulated | GO: 0051048 | negative | 139 | 15462 | 139 | 5 | 0.008405328 | 0.071182939 | RGCC, ADRA2A, IL6, PLA2R1, ADIPOQ |
| | | regulation of | | | | | | | |
| | | secretion | | | | | | | |
| upregulated | GO: 0010975 | regulation of | 139 | 15462 | 257 | 7 | 0.008611379 | 0.071182939 | LRRK2, SDC2, PDLIM5, TENM3, |
| | | neuron | | | | | | | SEMA5A, SHOX2, CNTN1 |
| | | projection | | | | | | | |
| | | development | | | | | | | |
| upregulated | GO: 0050871 | positive | 139 | 15462 | 47 | 3 | 0.008638028 | 0.071182939 | IL6, NFATC2, PTPRC |
| | | regulation of B | | | | | | | |
| | | cell activation | | | | | | | |
| upregulated | GO: 0050767 | regulation of | 139 | 15462 | 460 | 10 | 0.008657802 | 0.071182939 | LRRK2, GDF6, SDC2, TENM3, SEMA5A, |
| | | neurogenesis | | | | | | | SHOX2, BMP2, IL6, PDLIM5, CNTN1 |
| upregulated | GO: 0051179 | localization | 139 | 15462 | 4718 | 56 | 0.008795002 | 0.071182939 | GPC6, DCLK1, FABP4, ADRA2A, CD2, |
| | | | | | | | | | HOXA5, RAB30, NFATC2, BNIP3L, |
| | | | | | | | | | NKD1, NPTX1, IL18R1, DPP4, JAKMIP1, |
| | | | | | | | | | COL1A2, CAMK2D, FYB, ITGB3, RGCC, |
| | | | | | | | | | LCP2, ASIC2, CXCL9, SLC22A3, CNTN1, |
| | | | | | | | | | TSPAN5, TLR1, STEAP4, SEMA5A, |
| | | | | | | | | | CHRNA1, SOS1, PLA2R1, MDFIC, |
| | | | | | | | | | PIEZO2, CD36, MMP1, SATB2, ATP11A, |
| | | | | | | | | | UBAC2, LRRK2, STAP1, NR4A1, PRKG1, |
| | | | | | | | | | MEGF10, CDH13, CCR1, BMP2, PTPRC, |
| | | | | | | | | | TNFRSF11A, WNT4, CXCL5, IL6, TBX5, |
| | | | | | | | | | CBLN4, SLC24A3, SLC7A5, ADIPOQ |
| upregulated | GO: 0032387 | negative | 139 | 15462 | 90 | 4 | 0.008803541 | 0.071182939 | LRRK2, MDFIC, CD36, ADIPOQ |
| | | regulation of | | | | | | | |
| | | intracellular | | | | | | | |
| | | transport | | | | | | | |
| upregulated | GO: 0050707 | regulation of | 139 | 15462 | 90 | 4 | 0.008803541 | 0.071182939 | RGCC, CD2, IL6, TLR1 |
| | | cytokine | | | | | | | |
| | | secretion | | | | | | | |
| upregulated | GO: 0046716 | muscle cell | 139 | 15462 | 16 | 2 | 0.008865634 | 0.071182939 | IL6, CHRNA1 |
| | | cellular | | | | | | | |
| | | homeostasis | | | | | | | |
| upregulated | GO: 0010888 | negative | 139 | 15462 | 16 | 2 | 0.008865634 | 0.071182939 | ITGB3, IL6 |
| | | regulation of lipid | | | | | | | |
| | | storage | | | | | | | |
| upregulated | GO: 0008207 | C21-steroid | 139 | 15462 | 16 | 2 | 0.008865634 | 0.071182939 | WNT4, BMP2 |
| | | hormone | | | | | | | |
| | | metabolic | | | | | | | |
| | | process | | | | | | | |
| upregulated | GO: 0002360 | T cell lineage | 139 | 15462 | 16 | 2 | 0.008865634 | 0.071182939 | IL6, IRF4 |
| | | commitment | | | | | | | |
| upregulated | GO: 2000066 | positive | 139 | 15462 | 1 | 1 | 0.008989781 | 0.071182939 | WNT4 |
| | | regulation of | | | | | | | |
| | | cortisol | | | | | | | |
| | | biosynthetic | | | | | | | |
| | | process | | | | | | | |
| upregulated | GO: 1900139 | negative | 139 | 15462 | 1 | 1 | 0.008989781 | 0.071182939 | PLA2R1 |
| | | regulation of | | | | | | | |
| | | arachidonic acid | | | | | | | |
| | | secretion | | | | | | | |
| upregulated | GO: 0051042 | negative | 139 | 15462 | 1 | 1 | 0.008989781 | 0.071182939 | BMP2 |
| | | regulation of | | | | | | | |
| | | calcium- | | | | | | | |
| | | independent cell- | | | | | | | |
| | | cell adhesion | | | | | | | |
| upregulated | GO: 1901231 | positive | 139 | 15462 | 1 | 1 | 0.008989781 | 0.071182939 | NKD1 |
| | | regulation of | | | | | | | |
| | | non-canonical | | | | | | | |
| | | Wnt signaling | | | | | | | |
| | | pathway via JNK | | | | | | | |
| | | cascade | | | | | | | |
| upregulated | GO: 2000590 | negative | 139 | 15462 | 1 | 1 | 0.008989781 | 0.071182939 | ADIPOQ |
| | | regulation of | | | | | | | |
| | | metanephric | | | | | | | |
| | | mesenchymal | | | | | | | |
| | | cell migration | | | | | | | |
| upregulated | GO: 0060980 | cell migration | 139 | 15462 | 1 | 1 | 0.008989781 | 0.071182939 | TBX5 |
| | | involved in | | | | | | | |
| | | coronary | | | | | | | |
| | | vasculogenesis | | | | | | | |
| upregulated | GO: 0045368 | positive | 139 | 15462 | 1 | 1 | 0.008989781 | 0.071182939 | IRF4 |
| | | regulation of | | | | | | | |
| | | interleukin-13 | | | | | | | |
| | | biosynthetic | | | | | | | |
| | | process | | | | | | | |
| upregulated | GO: 0003026 | regulation of | 139 | 15462 | 1 | 1 | 0.008989781 | 0.071182939 | ASIC2 |
| | | systemic arterial | | | | | | | |
| | | blood pressure | | | | | | | |
| | | by aortic arch | | | | | | | |
| | | baroreceptor | | | | | | | |
| | | feedback | | | | | | | |
| upregulated | GO: 2000534 | positive | 139 | 15462 | 1 | 1 | 0.008989781 | 0.071182939 | ADIPOQ |
| | | regulation of | | | | | | | |
| | | renal albumin | | | | | | | |
| | | absorption | | | | | | | |
| upregulated | GO: 2000181 | negative | 139 | 15462 | 1 | 1 | 0.008989781 | 0.071182939 | WNT4 |
| | | regulation of | | | | | | | |
| | | blood vessel | | | | | | | |
| | | morphogenesis | | | | | | | |
| upregulated | GO: 2000584 | negative | 139 | 15462 | 1 | 1 | 0.008989781 | 0.071182939 | ADIPOQ |
| | | regulation of | | | | | | | |
| | | platelet-derived | | | | | | | |
| | | growth factor | | | | | | | |
| | | receptor-alpha | | | | | | | |
| | | signaling | | | | | | | |
| | | pathway | | | | | | | |
| upregulated | GO: 2000583 | regulation of | 139 | 15462 | 1 | 1 | 0.008989781 | 0.071182939 | ADIPOQ |
| | | platelet-derived | | | | | | | |
| | | growth factor | | | | | | | |
| | | receptor-alpha | | | | | | | |
| | | signaling | | | | | | | |
| | | pathway | | | | | | | |
| upregulated | GO: 0070994 | detection of | 139 | 15462 | 1 | 1 | 0.008989781 | 0.071182939 | ADIPOQ |
| | | oxidative stress | | | | | | | |
| upregulated | GO: 2000180 | negative | 139 | 15462 | 1 | 1 | 0.008989781 | 0.071182939 | WNT4 |
| | | regulation of | | | | | | | |
| | | androgen | | | | | | | |
| | | biosynthetic | | | | | | | |
| | | process | | | | | | | |
| upregulated | GO: 0045366 | regulation of | 139 | 15462 | 1 | 1 | 0.008989781 | 0.071182939 | IRF4 |
| | | interleukin-13 | | | | | | | |
| | | biosynthetic | | | | | | | |
| | | process | | | | | | | |
| upregulated | GO: 0002121 | inter-male | 139 | 15462 | 1 | 1 | 0.008989781 | 0.071182939 | GCNT4 |
| | | aggressive | | | | | | | |
| | | behavior | | | | | | | |
| upregulated | GO: 1902823 | negative | 139 | 15462 | 1 | 1 | 0.008989781 | 0.071182939 | LRRK2 |
| | | regulation of late | | | | | | | |
| | | endosome to | | | | | | | |
| | | lysosome | | | | | | | |
| | | transport | | | | | | | |
| upregulated | GO: 2000477 | regulation of | 139 | 15462 | 1 | 1 | 0.008989781 | 0.071182939 | ADIPOQ |
| | | metanephric | | | | | | | |
| | | glomerular | | | | | | | |
| | | visceral epithelial | | | | | | | |
| | | cell development | | | | | | | |
| upregulated | GO: 1902715 | positive | 139 | 15462 | 1 | 1 | 0.008989781 | 0.071182939 | CD2 |
| | | regulation of | | | | | | | |
| | | interferon- | | | | | | | |
| | | gamma secretion | | | | | | | |
| upregulated | GO: 1902713 | regulation of | 139 | 15462 | 1 | 1 | 0.008989781 | 0.071182939 | CD2 |
| | | interferon- | | | | | | | |
| | | gamma secretion | | | | | | | |
| upregulated | GO: 0060804 | positive | 139 | 15462 | 1 | 1 | 0.008989781 | 0.071182939 | BMP2 |
| | | regulation of Wnt | | | | | | | |
| | | signaling | | | | | | | |
| | | pathway by BMP | | | | | | | |
| | | signaling | | | | | | | |
| | | pathway | | | | | | | |
| upregulated | GO: 0061485 | memory T cell | 139 | 15462 | 1 | 1 | 0.008989781 | 0.071182939 | DOCK8 |
| | | proliferation | | | | | | | |
| upregulated | GO: 1901232 | regulation of | 139 | 15462 | 1 | 1 | 0.008989781 | 0.071182939 | NKD1 |
| | | convergent | | | | | | | |
| | | extension | | | | | | | |
| | | involved in axis | | | | | | | |
| | | elongation | | | | | | | |
| upregulated | GO: 0030887 | positive | 139 | 15462 | 1 | 1 | 0.008989781 | 0.071182939 | CD2 |
| | | regulation of | | | | | | | |
| | | myeloid dendritic | | | | | | | |
| | | cell activation | | | | | | | |
| upregulated | GO: 0031948 | positive | 139 | 15462 | 1 | 1 | 0.008989781 | 0.071182939 | WNT4 |
| | | regulation of | | | | | | | |
| | | glucocorticoid | | | | | | | |
| | | biosynthetic | | | | | | | |
| | | process | | | | | | | |
| upregulated | GO: 1901233 | negative | 139 | 15462 | 1 | 1 | 0.008989781 | 0.071182939 | NKD1 |
| | | regulation of | | | | | | | |
| | | convergent | | | | | | | |
| | | extension | | | | | | | |
| | | involved in axis | | | | | | | |
| | | elongation | | | | | | | |
| upregulated | GO: 1903125 | negative | 139 | 15462 | 1 | 1 | 0.008989781 | 0.071182939 | LRRK2 |
| | | regulation of | | | | | | | |
| | | thioredoxin | | | | | | | |
| | | peroxidase | | | | | | | |
| | | activity by | | | | | | | |
| | | peptidyl- | | | | | | | |
| | | threonine | | | | | | | |
| | | phosphorylation | | | | | | | |
| upregulated | GO: 1901229 | regulation of | 139 | 15462 | 1 | 1 | 0.008989781 | 0.071182939 | NKD1 |
| | | non-canonical | | | | | | | |
| | | Wnt signaling | | | | | | | |
| | | pathway via JNK | | | | | | | |
| | | cascade | | | | | | | |
| upregulated | GO: 0061369 | negative | 139 | 15462 | 1 | 1 | 0.008989781 | 0.071182939 | WNT4 |
| | | regulation of | | | | | | | |
| | | testicular blood | | | | | | | |
| | | vessel | | | | | | | |
| | | morphogenesis | | | | | | | |
| upregulated | GO: 0071882 | phospholipase | 139 | 15462 | 1 | 1 | 0.008989781 | 0.071182939 | ADRA2A |
| | | C-activating | | | | | | | |
| | | adrenergic | | | | | | | |
| | | receptor | | | | | | | |
| | | signaling | | | | | | | |
| | | pathway | | | | | | | |
| upregulated | GO: 2000225 | negative | 139 | 15462 | 1 | 1 | 0.008989781 | 0.071182939 | WNT4 |
| | | regulation of | | | | | | | |
| | | testosterone | | | | | | | |
| | | biosynthetic | | | | | | | |
| | | process | | | | | | | |
| upregulated | GO: 1990256 | signal clustering | 139 | 15462 | 1 | 1 | 0.008989781 | 0.071182939 | SEMA5A |
| upregulated | GO: 0031945 | positive | 139 | 15462 | 1 | 1 | 0.008989781 | 0.071182939 | WNT4 |
| | | regulation of | | | | | | | |
| | | glucocorticoid | | | | | | | |
| | | metabolic | | | | | | | |
| | | process | | | | | | | |
| upregulated | GO: 2000478 | positive | 139 | 15462 | 1 | 1 | 0.008989781 | 0.071182939 | ADIPOQ |
| | | regulation of | | | | | | | |
| | | metanephric | | | | | | | |
| | | glomerular | | | | | | | |
| | | visceral epithelial | | | | | | | |
| | | cell development | | | | | | | |
| upregulated | GO: 0001817 | regulation of | 139 | 15462 | 464 | 10 | 0.009168841 | 0.072409719 | BIRC3, TLR1, IL18R1, IRF4, RGCC, CD2, |
| | | cytokine | | | | | | | ADRA2A, IL6, CD36, ADIPOQ |
| | | production | | | | | | | |
| upregulated | GO: 0061041 | regulation of | 139 | 15462 | 92 | 4 | 0.009495841 | 0.074795321 | ADRA2A, WNT4, ASIC2, CD36 |
| | | wound healing | | | | | | | |
| upregulated | GO: 0009100 | glycoprotein | 139 | 15462 | 328 | 8 | 0.00963074 | 0.075659296 | GPC6, SDC2, GCNT4, GALNT5, BMP2, |
| | | metabolic | | | | | | | HS3ST3B1, GALNT14, CHSY3 |
| | | process | | | | | | | |
| upregulated | GO: 0042306 | regulation of | 139 | 15462 | 144 | 5 | 0.009705996 | 0.07605142 | IL6, MDFIC, IL18R1, SEMA5A, CD36 |
| | | protein import | | | | | | | |
| | | into nucleus | | | | | | | |
| upregulated | GO: 0042181 | ketone | 139 | 15462 | 17 | 2 | 0.009988942 | 0.076667046 | WNT4, BMP2 |
| | | biosynthetic | | | | | | | |
| | | process | | | | | | | |
| upregulated | GO: 0034383 | low-density | 139 | 15462 | 17 | 2 | 0.009988942 | 0.076667046 | CD36, ADIPOQ |
| | | lipoprotein | | | | | | | |
| | | particle | | | | | | | |
| | | clearance | | | | | | | |
| upregulated | GO: 0006704 | glucocorticoid | 139 | 15462 | 17 | 2 | 0.009988942 | 0.076667046 | WNT4, BMP2 |
| | | biosynthetic | | | | | | | |
| | | process | | | | | | | |
| upregulated | GO: 0060039 | pericardium | 139 | 15462 | 17 | 2 | 0.009988942 | 0.076667046 | TBX5, BMP2 |
| | | development | | | | | | | |
| upregulated | GO: 0061377 | mammary gland | 139 | 15462 | 17 | 2 | 0.009988942 | 0.076667046 | TNFRSF11A, HOXA5 |
| | | lobule | | | | | | | |
| | | development | | | | | | | |
| upregulated | GO: 0060749 | mammary gland | 139 | 15462 | 17 | 2 | 0.009988942 | 0.076667046 | TNFRSF11A, HOXA5 |
| | | alveolus | | | | | | | |
| | | development | | | | | | | |
| upregulated | GO: 0072207 | metanephric | 139 | 15462 | 17 | 2 | 0.009988942 | 0.076667046 | ADIPOQ, CALB1 |
| | | epithelium | | | | | | | |
| | | development | | | | | | | |
| upregulated | GO: 0090495 | low-density | 139 | 15462 | 17 | 2 | 0.009988942 | 0.076667046 | CD36, ADIPOQ |
| | | lipoprotein | | | | | | | |
| | | particle | | | | | | | |
| | | disassembly | | | | | | | |
| upregulated | GO: 0001819 | positive | 139 | 15462 | 265 | 7 | 0.0100897 | 0.077242829 | ADRA2A, CD2, BIRC3, IL6, IL18R1, |
| | | regulation of | | | | | | | CD36, ADIPOQ |
| | | cytokine | | | | | | | |
| | | production | | | | | | | |
| upregulated | GO: 0042325 | regulation of | 139 | 15462 | 1105 | 18 | 0.010152814 | 0.077528231 | FAM129A, CBLC, FABP4, ADRA2A, |
| | | phosphorylation | | | | | | | MDFIC, CD36, LRRK2, BIRC3, GDF6, |
| | | | | | | | | | CCR1, PTPRC, BMP2, TNFRSF11A, |
| | | | | | | | | | RGCC, ITGB3, IL6, ADIPOQ, FOXO1 |
| upregulated | GO: 0061035 | regulation of | 139 | 15462 | 50 | 3 | 0.010239708 | 0.077993309 | EFEMP1, SHOX2, BMP2 |
| | | cartilage | | | | | | | |
| | | development | | | | | | | |
| upregulated | GO: 0043405 | regulation of | 139 | 15462 | 266 | 7 | 0.010286739 | 0.078153177 | ADRA2A, TNFRSF11A, LRRK2, MDFIC, |
| | | MAP kinase | | | | | | | CBLC, ADIPOQ, BMP2 |
| | | activity | | | | | | | |
| upregulated | GO: 0051336 | regulation of | 139 | 15462 | 1108 | 18 | 0.01042223 | 0.078982609 | TAGAP, SOS1, ADRA2A, PLA2R1, |
| | | hydrolase activity | | | | | | | DOCK8, LRRK2, BIRC3, NR4A1, PRKG1, |
| | | | | | | | | | PDE1A, RASGRF2, ANGPTL4, CAMK2D, |
| | | | | | | | | | BMP2, WNT4, IL6, TFPI2, ARHGAP42 |
| upregulated | GO: 0044057 | regulation of | 139 | 15462 | 333 | 8 | 0.010488174 | 0.079282139 | ADRA2A, FBXO32, IL6, CELF2, ASIC2, |
| | | system process | | | | | | | GUCY1A3, ADIPOQ, CAMK2D |
| upregulated | GO: 0016358 | dendrite | 139 | 15462 | 147 | 5 | 0.010549546 | 0.079545699 | LRRK2, PRKG1, PDLIM5, SDC2, DCLK1 |
| | | development | | | | | | | |
| upregulated | GO: 0010876 | lipid localization | 139 | 15462 | 268 | 7 | 0.010689229 | 0.080195938 | TNFRSF11A, ITGB3, ATP11A, PLA2R1, |
| | | | | | | | | | IL6, CD36, ADIPOQ |
| upregulated | GO: 0042692 | muscle cell | 139 | 15462 | 268 | 7 | 0.010689229 | 0.080195938 | FBXO40, WNT4, PRDM6, TBX5, |
| | | differentiation | | | | | | | MEGF10, SHOX2, BMP2 |
| upregulated | GO: 0010717 | regulation of | 139 | 15462 | 51 | 3 | 0.010809284 | 0.080693184 | RGCC, TBX5, BMP2 |
| | | epithelial to | | | | | | | |
| | | mesenchymal | | | | | | | |
| | | transition | | | | | | | |
| upregulated | GO: 0072009 | nephron | 139 | 15462 | 51 | 3 | 0.010809284 | 0.080693184 | WNT4, ADIPOQ, CALB1 |
| | | epithelium | | | | | | | |
| | | development | | | | | | | |
| upregulated | GO: 0001503 | ossification | 139 | 15462 | 335 | 8 | 0.010846578 | 0.080770668 | TNFRSF11A, WNT4, IL6, DLX5, SHOX2, |
| | | | | | | | | | CCR1, BMP2, SATB2 |
| upregulated | GO: 1900180 | regulation of | 139 | 15462 | 149 | 5 | 0.011139025 | 0.082578282 | IL6, MDFIC, IL18R1, SEMA5A, CD36 |
| | | protein | | | | | | | |
| | | localization to | | | | | | | |
| | | nucleus | | | | | | | |
| upregulated | GO: 0003171 | atrioventricular | 139 | 15462 | 18 | 2 | 0.01117187 | 0.082578282 | TBX5, BMP2 |
| | | valve | | | | | | | |
| | | development | | | | | | | |
| upregulated | GO: 0010894 | negative | 139 | 15462 | 18 | 2 | 0.01117187 | 0.082578282 | WNT4, BMP2 |
| | | regulation of | | | | | | | |
| | | steroid | | | | | | | |
| | | biosynthetic | | | | | | | |
| | | process | | | | | | | |
| upregulated | GO: 0002757 | immune | 139 | 15462 | 337 | 8 | 0.011213995 | 0.082685992 | LCP2, BIRC3, TLR1, NFATC2, IRF4, |
| | | response- | | | | | | | CD36, PTPRC, FYB |
| | | activating signal | | | | | | | |
| | | transduction | | | | | | | |
| upregulated | GO: 0016310 | phosphorylation | 139 | 15462 | 1733 | 25 | 0.011323334 | 0.082813616 | FAM129A, CBLC, DCLK1, FABP4, |
| | | | | | | | | | ADRA2A, EFEMP1, MDFIC, CD36, |
| | | | | | | | | | LRRK2, NKD1, BIRC3, GDF6, PRKG1, |
| | | | | | | | | | CCR1, BMP2, FYB, CAMK2D, PTPRC, |
| | | | | | | | | | TNFRSF11A, ITGB3, RGCC, WNT4, IL6, |
| | | | | | | | | | FOXO1, ADIPOQ |
| upregulated | GO: 0051384 | response to | 139 | 15462 | 97 | 4 | 0.011378978 | 0.082813616 | PAPPA, IL6, BCHE, ADIPOQ |
| | | glucocorticoid | | | | | | | |
| upregulated | GO: 0048814 | regulation of | 139 | 15462 | 52 | 3 | 0.011396876 | 0.082813616 | LRRK2, PDLIM5, SDC2 |
| | | dendrite | | | | | | | |
| | | morphogenesis | | | | | | | |
| upregulated | GO: 0006027 | glycosaminoglycan | 139 | 15462 | 52 | 3 | 0.011396876 | 0.082813616 | GPC6, PRELP, SDC2 |
| | | catabolic | | | | | | | |
| | | process | | | | | | | |
| upregulated | GO: 0030101 | natural killer cell | 139 | 15462 | 52 | 3 | 0.011396876 | 0.082813616 | CD2, SLAMF7, IL18R1 |
| | | activation | | | | | | | |
| upregulated | GO: 0006026 | aminoglycan | 139 | 15462 | 52 | 3 | 0.011396876 | 0.082813616 | GPC6, PRELP, SDC2 |
| | | catabolic | | | | | | | |
| | | process | | | | | | | |
| upregulated | GO: 0070372 | regulation of | 139 | 15462 | 151 | 5 | 0.011750628 | 0.085177863 | TNFRSF11A, IL6, CCR1, ADIPOQ, BMP2 |
| | | ERK1 and ERK2 | | | | | | | |
| | | cascade | | | | | | | |
| upregulated | GO: 0012501 | programmed cell | 139 | 15462 | 1651 | 24 | 0.011995691 | 0.086744743 | NR4A3, SOS1, SEMA5A, CD2, HOXA5, |
| | | death | | | | | | | FEM1B, BNIP3L, LRRK2, BIRC3, NR4A1, |
| | | | | | | | | | GDF6, NPTX1, RASGRF2, PDE1A, |
| | | | | | | | | | ANGPTL4, PNMA2, BMP2, RGCC, WNT4, |
| | | | | | | | | | IL6, TBX5, ASIC2, FOXO1, ADIPOQ |
| upregulated | GO: 0080135 | regulation of | 139 | 15462 | 342 | 8 | 0.012172828 | 0.08781408 | TNFRSF11A, NPAS2, MDFIC, PLA2R1, |
| | | cellular response | | | | | | | FEM1B, CD36, BMP2, FOXO1 |
| | | to stress | | | | | | | |
| upregulated | GO: 0019932 | second- | 139 | 15462 | 343 | 8 | 0.012371631 | 0.088284405 | ADRA2A, CXCL9, CDH13, MCTP1, CD36, |
| | | messenger- | | | | | | | CCR1, GUCY1A3, CAMK2D |
| | | mediated | | | | | | | |
| | | signaling | | | | | | | |
| upregulated | GO: 0045742 | positive | 139 | 15462 | 19 | 2 | 0.012413272 | 0.088284405 | ADRA2A, SOS1 |
| | | regulation of | | | | | | | |
| | | epidermal growth | | | | | | | |
| | | factor receptor | | | | | | | |
| | | signaling | | | | | | | |
| | | pathway | | | | | | | |
| upregulated | GO: 2000352 | negative | 139 | 15462 | 19 | 2 | 0.012413272 | 0.088284405 | SEMA5A, ANGPTL4 |
| | | regulation of | | | | | | | |
| | | endothelial cell | | | | | | | |
| | | apoptotic | | | | | | | |
| | | process | | | | | | | |
| upregulated | GO: 0045932 | negative | 139 | 15462 | 19 | 2 | 0.012413272 | 0.088284405 | ADRA2A, GUCY1A3 |
| | | regulation of | | | | | | | |
| | | muscle | | | | | | | |
| | | contraction | | | | | | | |
| upregulated | GO: 0007263 | nitric oxide | 139 | 15462 | 19 | 2 | 0.012413272 | 0.088284405 | CD36, GUCY1A3 |
| | | mediated signal | | | | | | | |
| | | transduction | | | | | | | |
| upregulated | GO: 0098602 | single organism | 139 | 15462 | 276 | 7 | 0.012414535 | 0.088284405 | RGCC, CD2, WNT4, MEGF10, DPP4, |
| | | cell adhesion | | | | | | | ADIPOQ, BMP2 |
| upregulated | GO: 0031401 | positive | 139 | 15462 | 797 | 14 | 0.012538238 | 0.088953318 | FAM129A, LRRK2, BIRC3, GDF6, BMP2, |
| | | regulation of | | | | | | | PTPRC, RGCC, TNFRSF11A, ADRA2A, |
| | | protein | | | | | | | ITGB3, IL6, MDFIC, CD36, ADIPOQ |
| | | modification | | | | | | | |
| | | process | | | | | | | |
| upregulated | GO: 0048520 | positive | 139 | 15462 | 100 | 4 | 0.012616379 | 0.089157789 | IL6, CDH13, SEMA5A, CCR1 |
| | | regulation of | | | | | | | |
| | | behavior | | | | | | | |
| upregulated | GO: 0002040 | sprouting | 139 | 15462 | 54 | 3 | 0.012626478 | 0.089157789 | NR4A1, CDH13, SEMA5A |
| | | angiogenesis | | | | | | | |
| upregulated | GO: 0009628 | response to | 139 | 15462 | 880 | 15 | 0.012734503 | 0.08970949 | GPC6, SDC2, ANGPTL4, DPP4, BMP2, |
| | | abiotic stimulus | | | | | | | RGCC, TNFRSF11A, IL6, ERCC1, ASIC2, |
| | | | | | | | | | TFEC, FOSL2, PIEZO2, ADIPOQ, FOXO1 |
| upregulated | GO: 0016266 | O-glycan | 139 | 15462 | 55 | 3 | 0.013268655 | 0.093035595 | GALNT14, GCNT4, GALNT5 |
| | | processing | | | | | | | |
| upregulated | GO: 0046888 | negative | 139 | 15462 | 55 | 3 | 0.013268655 | 0.093035595 | ADRA2A, IL6, ADIPOQ |
| | | regulation of | | | | | | | |
| | | hormone | | | | | | | |
| | | secretion | | | | | | | |
| upregulated | GO: 0002764 | immune | 139 | 15462 | 348 | 8 | 0.013401719 | 0.093046824 | LCP2, BIRC3, TLR1, NFATC2, IRF4, |
| | | response- | | | | | | | CD36, PTPRC, FYB |
| | | regulating | | | | | | | |
| | | signaling | | | | | | | |
| | | pathway | | | | | | | |
| upregulated | GO: 0007411 | axon guidance | 139 | 15462 | 348 | 8 | 0.013401719 | 0.093046824 | NR4A3, DLX5, GFRA1, SEMA5A, SOS1, |
| | | | | | | | | | PTPRC, ITGB3, CNTN1 |
| upregulated | GO: 0097485 | neuron | 139 | 15462 | 348 | 8 | 0.013401719 | 0.093046824 | NR4A3, DLX5, GFRA1, SEMA5A, SOS1, |
| | | projection | | | | | | | PTPRC, ITGB3, CNTN1 |
| | | guidance | | | | | | | |
| upregulated | GO: 0002682 | regulation of | 139 | 15462 | 969 | 16 | 0.013480638 | 0.093046824 | SLAMF7, BIRC3, TLR1, IRF4, DPP4, |
| | | immune system | | | | | | | CCR1, FYB, PTPRC, RGCC, CD2, LCP2, |
| | | process | | | | | | | IL6, HOXA5, NFATC2, CD36, ADIPOQ |
| upregulated | GO: 0045667 | regulation of | 139 | 15462 | 102 | 4 | 0.013487294 | 0.093046824 | WNT4, IL6, DLX5, BMP2 |
| | | osteoblast | | | | | | | |
| | | differentiation | | | | | | | |
| upregulated | GO: 0051147 | regulation of | 139 | 15462 | 102 | 4 | 0.013487294 | 0.093046824 | PRDM6, MEGF10, SHOX2, BMP2 |
| | | muscle cell | | | | | | | |
| | | differentiation | | | | | | | |
| upregulated | GO: 0045732 | positive | 139 | 15462 | 102 | 4 | 0.013487294 | 0.093046824 | ADRA2A, LRRK2, NKD1, FOXO1 |
| | | regulation of | | | | | | | |
| | | protein catabolic | | | | | | | |
| | | process | | | | | | | |
| upregulated | GO: 0001655 | urogenital | 139 | 15462 | 281 | 7 | 0.013590097 | 0.093098999 | WNT4, LRRK2, FEM1B, GCNT4, CALB1, |
| | | system | | | | | | | ADIPOQ, BMP2 |
| | | development | | | | | | | |
| upregulated | GO: 0043410 | positive | 139 | 15462 | 349 | 8 | 0.013615064 | 0.093098999 | LRRK2, CCR1, BMP2, TNFRSF11A, |
| | | regulation of | | | | | | | ADRA2A, IL6, MDFIC, CD36 |
| | | MAPK cascade | | | | | | | |
| upregulated | GO: 0006111 | regulation of | 139 | 15462 | 20 | 2 | 0.013712015 | 0.093098999 | IL6, ADIPOQ |
| | | gluconeogenesis | | | | | | | |
| upregulated | GO: 0046885 | regulation of | 139 | 15462 | 20 | 2 | 0.013712015 | 0.093098999 | WNT4, BMP2 |
| | | hormone | | | | | | | |
| | | biosynthetic | | | | | | | |
| | | process | | | | | | | |
| upregulated | GO: 1901186 | positive | 139 | 15462 | 20 | 2 | 0.013712015 | 0.093098999 | ADRA2A, SOS1 |
| | | regulation of | | | | | | | |
| | | ERBB signaling | | | | | | | |
| | | pathway | | | | | | | |
| upregulated | GO: 0045939 | negative | 139 | 15462 | 20 | 2 | 0.013712015 | 0.093098999 | WNT4, BMP2 |
| | | regulation of | | | | | | | |
| | | steroid metabolic | | | | | | | |
| | | process | | | | | | | |
| upregulated | GO: 0061001 | regulation of | 139 | 15462 | 20 | 2 | 0.013712015 | 0.093098999 | LRRK2, PDLIM5 |
| | | dendritic spine | | | | | | | |
| | | morphogenesis | | | | | | | |
| upregulated | GO: 0019915 | lipid storage | 139 | 15462 | 56 | 3 | 0.013929183 | 0.094359998 | ITGB3, IL6, CD36 |
| upregulated | GO: 0031960 | response to | 139 | 15462 | 104 | 4 | 0.014395611 | 0.097179955 | PAPPA, IL6, BCHE, ADIPOQ |
| | | corticosteroid | | | | | | | |
| upregulated | GO: 0032880 | regulation of | 139 | 15462 | 424 | 9 | 0.01444223 | 0.097179955 | TLR1, IL18R1, SEMA5A, CD2, RGCC, |
| | | protein | | | | | | | IL6, MDFIC, CD36, ADIPOQ |
| | | localization | | | | | | | |
| upregulated | GO: 0065009 | regulation of | 139 | 15462 | 2424 | 32 | 0.014442606 | 0.097179955 | TAGAP, CBLC, IRF4, FABP4, ADRA2A, |
| | | molecular | | | | | | | IL18R1, RASGRF2, CAMK2D, ITGB3, |
| | | function | | | | | | | RGCC, TFPI2, ARHGAP42, SOS1, |
| | | | | | | | | | RUNX1T1, MDFIC, PLA2R1, PPP1R3B, |
| | | | | | | | | | FEM1B, ALOX5AP, DOCK8, LRRK2, |
| | | | | | | | | | BIRC3, PRKG1, NR4A1, PDE1A, |
| | | | | | | | | | ANGPTL4, BMP2, PTPRC, TNFRSF11A, |
| | | | | | | | | | WNT4, IL6, ADIPOQ |
| upregulated | GO: 0045833 | negative | 139 | 15462 | 57 | 3 | 0.014608128 | 0.098073811 | ADRA2A, WNT4, BMP2 |
| | | regulation of lipid | | | | | | | |
| | | metabolic | | | | | | | |
| | | process | | | | | | | |
| upregulated | GO: 0002285 | lymphocyte | 139 | 15462 | 105 | 4 | 0.014863954 | 0.099375872 | IL6, ERCC1, IL18R1, IRF4 |
| | | activation | | | | | | | |
| | | involved in | | | | | | | |
| | | immune | | | | | | | |
| | | response | | | | | | | |
| upregulated | GO: 0031399 | regulation of | 139 | 15462 | 1151 | 18 | 0.01495358 | 0.099375872 | FAM129A, CBLC, FABP4, ADRA2A, |
| | | protein | | | | | | | MDFIC, FEM1B, CD36, LRRK2, BIRC3, |
| | | modification | | | | | | | GDF6, CAMK2D, PTPRC, BMP2, |
| | | process | | | | | | | TNFRSF11A, ITGB3, RGCC, IL6, |
| | | | | | | | | | ADIPOQ |
| upregulated | GO: 0019222 | regulation of | 139 | 15462 | 5790 | 65 | 0.015012091 | 0.099375872 | TAGAP, FAM129A, SHOX2, IRF4, |
| | | metabolic | | | | | | | FABP4, TNNI2, TFEC, NPAS2, ASIC2, |
| | | process | | | | | | | FOXO1, ARHGAP42, CNTN1, TLR1, |
| | | | | | | | | | SOS1, PLA2R1, MDFIC, FEM1B, |
| | | | | | | | | | ALOX5AP, FOSL2, CD36, SATB2, |
| | | | | | | | | | ZFHX4, PRKG1, PDE1A, GUCY1A3, |
| | | | | | | | | | TNFRSF11A, IL6, TBX5, CBLC, ADRA2A, |
| | | | | | | | | | HOXA5, IGJ, NFATC2, NKD1, GDF6, |
| | | | | | | | | | IL18R1, RASGRF2, DHX34, CAMK2D, |
| | | | | | | | | | HIVEP3, RGCC, ITGB3, PRDM6, BNC2, |
| | | | | | | | | | ERCC1, CXCL9, TFPI2, NR4A3, RFX2, |
| | | | | | | | | | RUNX1T1, EFEMP1, PPP1R3B, DOCK8, |
| | | | | | | | | | LRRK2, BIRC3, NR4A1, DLX5, CDH13, |
| | | | | | | | | | ANGPTL4, PTPRC, CCR1, BMP2, WNT4, |
| | | | | | | | | | NAMPT, ADIPOQ |
| upregulated | GO: 0071772 | response to BMP | 139 | 15462 | 21 | 2 | 0.015066985 | 0.099375872 | DLX5, BMP2 |
| upregulated | GO: 0090075 | relaxation of | 139 | 15462 | 21 | 2 | 0.015066985 | 0.099375872 | GUCY1A3, CAMK2D |
| | | muscle | | | | | | | |
| upregulated | GO: 0050805 | negative | 139 | 15462 | 21 | 2 | 0.015066985 | 0.099375872 | BCHE, ADIPOQ |
| | | regulation of | | | | | | | |
| | | synaptic | | | | | | | |
| | | transmission | | | | | | | |
| upregulated | GO: 0042745 | circadian | 139 | 15462 | 21 | 2 | 0.015066985 | 0.099375872 | NPAS2, IL6 |
| | | sleep/wake cycle | | | | | | | |
| upregulated | GO: 0071773 | cellular response | 139 | 15462 | 21 | 2 | 0.015066985 | 0.099375872 | DLX5, BMP2 |
| | | to BMP stimulus | | | | | | | |
| upregulated | GO: 0006606 | protein import | 139 | 15462 | 222 | 6 | 0.015169908 | 0.099399332 | IL6, MDFIC, IL18R1, SEMA5A, CD36, |
| | | into nucleus | | | | | | | FYB |
| upregulated | GO: 0044744 | protein targeting | 139 | 15462 | 222 | 6 | 0.015169908 | 0.099399332 | IL6, MDFIC, IL18R1, SEMA5A, CD36, |
| | | to nucleus | | | | | | | FYB |
| upregulated | GO: 1902593 | single-organism | 139 | 15462 | 222 | 6 | 0.015169908 | 0.099399332 | IL6, MDFIC, IL18R1, SEMA5A, CD36, |
| | | nuclear import | | | | | | | FYB |
| upregulated | GO: 0030204 | chondroitin | 139 | 15462 | 58 | 3 | 0.015305551 | 0.100069623 | GPC6, CHSY3, SDC2 |
| | | sulfate metabolic | | | | | | | |
| | | process | | | | | | | |
| upregulated | GO: 0046822 | regulation of | 139 | 15462 | 163 | 5 | 0.015904817 | 0.101611538 | IL6, MDFIC, IL18R1, SEMA5A, CD36 |
| | | nucleocytoplasmic | | | | | | | |
| | | transport | | | | | | | |
| upregulated | GO: 0043149 | stress fiber | 139 | 15462 | 59 | 3 | 0.016021503 | 0.101611538 | RGCC, ELN, WNT4 |
| | | assembly | | | | | | | |
| upregulated | GO: 0051170 | nuclear import | 139 | 15462 | 225 | 6 | 0.01610584 | 0.101611538 | IL6, MDFIC, IL18R1, SEMA5A, CD36, |
| | | | | | | | | | FYB |
| upregulated | GO: 0008219 | cell death | 139 | 15462 | 1880 | 26 | 0.016279069 | 0.101611538 | NR4A3, SOS1, SEMA5A, CD2, HOXA5, |
| | | | | | | | | | FEM1B, BNIP3L, FOSL2, LRRK2, NPAS2, |
| | | | | | | | | | BIRC3, NR4A1, GDF6, NPTX1, |
| | | | | | | | | | RASGRF2, PDE1A, ANGPTL4, PNMA2, |
| | | | | | | | | | BMP2, RGCC, WNT4, IL6, TBX5, ASIC2, |
| | | | | | | | | | FOXO1, ADIPOQ |
| upregulated | GO: 0070371 | ERK1 and ERK2 | 139 | 15462 | 164 | 5 | 0.016289875 | 0.101611538 | TNFRSF11A, IL6, CCR1, ADIPOQ, BMP2 |
| | | cascade | | | | | | | |
| upregulated | GO: 0002440 | production of | 139 | 15462 | 108 | 4 | 0.016326434 | 0.101611538 | IL6, ERCC1, CD36, PTPRC |
| | | molecular | | | | | | | |
| | | mediator of | | | | | | | |
| | | immune | | | | | | | |
| | | response | | | | | | | |
| upregulated | GO: 2000191 | regulation of fatty | 139 | 15462 | 22 | 2 | 0.01647708 | 0.101611538 | TNFRSF11A, PLA2R1 |
| | | acid transport | | | | | | | |
| upregulated | GO: 0055024 | regulation of | 139 | 15462 | 22 | 2 | 0.01647708 | 0.101611538 | TBX5, BMP2 |
| | | cardiac muscle | | | | | | | |
| | | tissue | | | | | | | |
| | | development | | | | | | | |
| upregulated | GO: 0003179 | heart valve | 139 | 15462 | 22 | 2 | 0.01647708 | 0.101611538 | TBX5, BMP2 |
| | | morphogenesis | | | | | | | |
| upregulated | GO: 0032892 | positive | 139 | 15462 | 22 | 2 | 0.01647708 | 0.101611538 | TNFRSF11A, PLA2R1 |
| | | regulation of | | | | | | | |
| | | organic acid | | | | | | | |
| | | transport | | | | | | | |
| upregulated | GO: 0045622 | regulation of T- | 139 | 15462 | 22 | 2 | 0.01647708 | 0.101611538 | IL6, IRF4 |
| | | helper cell | | | | | | | |
| | | differentiation | | | | | | | |
| upregulated | GO: 0032967 | positive | 139 | 15462 | 22 | 2 | 0.01647708 | 0.101611538 | RGCC, WNT4 |
| | | regulation of | | | | | | | |
| | | collagen | | | | | | | |
| | | biosynthetic | | | | | | | |
| | | process | | | | | | | |
| upregulated | GO: 0016265 | death | 139 | 15462 | 1883 | 26 | 0.016586739 | 0.101611538 | NR4A3, SOS1, SEMA5A, CD2, HOXA5, |
| | | | | | | | | | FEM1B, BNIP3L, FOSL2, LRRK2, NPAS2, |
| | | | | | | | | | BIRC3, NR4A1, GDF6, NPTX1, |
| | | | | | | | | | RASGRF2, PDE1A, ANGPTL4, PNMA2, |
| | | | | | | | | | BMP2, RGCC, WNT4, IL6, TBX5, ASIC2, |
| | | | | | | | | | FOXO1, ADIPOQ |
| upregulated | GO: 0030278 | regulation of | 139 | 15462 | 165 | 5 | 0.016681102 | 0.101611538 | WNT4, IL6, DLX5, CCR1, BMP2 |
| | | ossification | | | | | | | |
| upregulated | GO: 0007266 | Rho protein | 139 | 15462 | 227 | 6 | 0.016751574 | 0.101611538 | ADRA2A, WNT4, CDH13, RASGRF2, |
| | | signal | | | | | | | SOS1, COL1A2 |
| | | transduction | | | | | | | |
| upregulated | GO: 0050654 | chondroitin | 139 | 15462 | 60 | 3 | 0.016756034 | 0.101611538 | GPC6, CHSY3, SDC2 |
| | | sulfate | | | | | | | |
| | | proteoglycan | | | | | | | |
| | | metabolic | | | | | | | |
| | | process | | | | | | | |
| upregulated | GO: 0043255 | regulation of | 139 | 15462 | 60 | 3 | 0.016756034 | 0.101611538 | IL6, PPP1R3B, ADIPOQ |
| | | carbohydrate | | | | | | | |
| | | biosynthetic | | | | | | | |
| | | process | | | | | | | |
| upregulated | GO: 0002286 | T cell activation | 139 | 15462 | 60 | 3 | 0.016756034 | 0.101611538 | IL6, IL18R1, IRF4 |
| | | involved in | | | | | | | |
| | | immune | | | | | | | |
| | | response | | | | | | | |
| upregulated | GO: 0003012 | muscle system | 139 | 15462 | 294 | 7 | 0.017018123 | 0.101611538 | ADRA2A, TNNI2, FBXO32, CHRNA1, |
| | | process | | | | | | | LMOD1, GUCY1A3, CAMK2D |
| upregulated | GO: 0051607 | defense | 139 | 15462 | 166 | 5 | 0.017078536 | 0.101611538 | IL6, BIRC3, BNIP3L, CXCL9, PTPRC |
| | | response to virus | | | | | | | |
| upregulated | GO: 0001932 | regulation of | 139 | 15462 | 912 | 15 | 0.0171161 | 0.101611538 | FAM129A, LRRK2, CBLC, GDF6, BMP2, |
| | | protein | | | | | | | FABP4, PTPRC, RGCC, TNFRSF11A, |
| | | phosphorylation | | | | | | | ITGB3, ADRA2A, IL6, MDFIC, CD36, |
| | | | | | | | | | ADIPOQ |
| upregulated | GO: 0002673 | regulation of | 139 | 15462 | 61 | 3 | 0.017509183 | 0.101611538 | TNFRSF11A, IL6, ALOX5AP |
| | | acute | | | | | | | |
| | | inflammatory | | | | | | | |
| | | response | | | | | | | |
| upregulated | GO: 0050727 | regulation of | 139 | 15462 | 230 | 6 | 0.017753388 | 0.101611538 | TNFRSF11A, IL6, BIRC3, ALOX5AP, |
| | | inflammatory | | | | | | | ADIPOQ, FABP4 |
| | | response | | | | | | | |
| upregulated | GO: 0006468 | protein | 139 | 15462 | 1349 | 20 | 0.017831568 | 0.101611538 | FAM129A, CBLC, DCLK1, FABP4, |
| | | phosphorylation | | | | | | | ADRA2A, EFEMP1, MDFIC, CD36, |
| | | | | | | | | | LRRK2, GDF6, PRKG1, FYB, CAMK2D, |
| | | | | | | | | | PTPRC, BMP2, TNFRSF11A, ITGB3, |
| | | | | | | | | | RGCC, IL6, ADIPOQ |
| upregulated | GO: 0045082 | positive | 139 | 15462 | 2 | 1 | 0.017899323 | 0.101611538 | IRF4 |
| | | regulation of | | | | | | | |
| | | interleukin-10 | | | | | | | |
| | | biosynthetic | | | | | | | |
| | | process | | | | | | | |
| upregulated | GO: 0003218 | cardiac left | 139 | 15462 | 2 | 1 | 0.017899323 | 0.101611538 | TBX5 |
| | | ventricle | | | | | | | |
| | | formation | | | | | | | |
| upregulated | GO: 2000532 | regulation of | 139 | 15462 | 2 | 1 | 0.017899323 | 0.101611538 | ADIPOQ |
| | | renal albumin | | | | | | | |
| | | absorption | | | | | | | |
| upregulated | GO: 2000019 | negative | 139 | 15462 | 2 | 1 | 0.017899323 | 0.101611538 | WNT4 |
| | | regulation of | | | | | | | |
| | | male gonad | | | | | | | |
| | | development | | | | | | | |
| upregulated | GO: 0030237 | female sex | 139 | 15462 | 2 | 1 | 0.017899323 | 0.101611538 | WNT4 |
| | | determination | | | | | | | |
| upregulated | GO: 0071848 | positive | 139 | 15462 | 2 | 1 | 0.017899323 | 0.101611538 | TNFRSF11A |
| | | regulation of | | | | | | | |
| | | ERK1 and ERK2 | | | | | | | |
| | | cascade via | | | | | | | |
| | | TNFSF11- | | | | | | | |
| | | mediated | | | | | | | |
| | | signaling | | | | | | | |
| upregulated | GO: 0070473 | negative | 139 | 15462 | 2 | 1 | 0.017899323 | 0.101611538 | ADRA2A |
| | | regulation of | | | | | | | |
| | | uterine smooth | | | | | | | |
| | | muscle | | | | | | | |
| | | contraction | | | | | | | |
| upregulated | GO: 0060086 | circadian | 139 | 15462 | 2 | 1 | 0.017899323 | 0.101611538 | TNFRSF11A |
| | | temperature | | | | | | | |
| | | homeostasis | | | | | | | |
| upregulated | GO: 0060748 | tertiary | 139 | 15462 | 2 | 1 | 0.017899323 | 0.101611538 | WNT4 |
| | | branching | | | | | | | |
| | | involved in | | | | | | | |
| | | mammary gland | | | | | | | |
| | | duct | | | | | | | |
| | | morphogenesis | | | | | | | |
| upregulated | GO: 0042495 | detection of | 139 | 15462 | 2 | 1 | 0.017899323 | 0.101611538 | TLR1 |
| | | triacyl bacterial | | | | | | | |
| | | lipopeptide | | | | | | | |
| upregulated | GO: 0060979 | vasculogenesis | 139 | 15462 | 2 | 1 | 0.017899323 | 0.101611538 | TBX5 |
| | | involved in | | | | | | | |
| | | coronary | | | | | | | |
| | | vascular | | | | | | | |
| | | morphogenesis | | | | | | | |
| upregulated | GO: 2000676 | positive | 139 | 15462 | 2 | 1 | 0.017899323 | 0.101611538 | IL6 |
| | | regulation of type | | | | | | | |
| | | B pancreatic cell | | | | | | | |
| | | apoptotic | | | | | | | |
| | | process | | | | | | | |
| upregulated | GO: 0052551 | response to | 139 | 15462 | 2 | 1 | 0.017899323 | 0.101611538 | GUCY1A3 |
| | | defense-related | | | | | | | |
| | | nitric oxide | | | | | | | |
| | | production by | | | | | | | |
| | | other organism | | | | | | | |
| | | involved in | | | | | | | |
| | | symbiotic | | | | | | | |
| | | interaction | | | | | | | |
| upregulated | GO: 2000224 | regulation of | 139 | 15462 | 2 | 1 | 0.017899323 | 0.101611538 | WNT4 |
| | | testosterone | | | | | | | |
| | | biosynthetic | | | | | | | |
| | | process | | | | | | | |
| upregulated | GO: 0014016 | neuroblast | 139 | 15462 | 2 | 1 | 0.017899323 | 0.101611538 | BCHE |
| | | differentiation | | | | | | | |
| upregulated | GO: 0051040 | regulation of | 139 | 15462 | 2 | 1 | 0.017899323 | 0.101611538 | BMP2 |
| | | calcium- | | | | | | | |
| | | independent cell- | | | | | | | |
| | | cell adhesion | | | | | | | |
| upregulated | GO: 0090272 | negative | 139 | 15462 | 2 | 1 | 0.017899323 | 0.101611538 | RGCC |
| | | regulation of | | | | | | | |
| | | fibroblast growth | | | | | | | |
| | | factor production | | | | | | | |
| upregulated | GO: 0002384 | hepatic immune | 139 | 15462 | 2 | 1 | 0.017899323 | 0.101611538 | IL6 |
| | | response | | | | | | | |
| upregulated | GO: 1902774 | late endosome to | 139 | 15462 | 2 | 1 | 0.017899323 | 0.101611538 | LRRK2 |
| | | lysosome | | | | | | | |
| | | transport | | | | | | | |
| upregulated | GO: 0042231 | interleukin-13 | 139 | 15462 | 2 | 1 | 0.017899323 | 0.101611538 | IRF4 |
| | | biosynthetic | | | | | | | |
| | | process | | | | | | | |
| upregulated | GO: 0097017 | renal protein | 139 | 15462 | 2 | 1 | 0.017899323 | 0.101611538 | ADIPOQ |
| | | absorption | | | | | | | |
| upregulated | GO: 0071810 | regulation of | 139 | 15462 | 2 | 1 | 0.017899323 | 0.101611538 | TNFRSF11A |
| | | fever generation | | | | | | | |
| | | by regulation of | | | | | | | |
| | | prostaglandin | | | | | | | |
| | | secretion | | | | | | | |
| upregulated | GO: 0032349 | positive | 139 | 15462 | 2 | 1 | 0.017899323 | 0.101611538 | WNT4 |
| | | regulation of | | | | | | | |
| | | aldosterone | | | | | | | |
| | | biosynthetic | | | | | | | |
| | | process | | | | | | | |
| upregulated | GO: 0071847 | TNFSF11- | 139 | 15462 | 2 | 1 | 0.017899323 | 0.101611538 | TNFRSF11A |
| | | mediated | | | | | | | |
| | | signaling | | | | | | | |
| | | pathway | | | | | | | |
| upregulated | GO: 1900138 | negative | 139 | 15462 | 2 | 1 | 0.017899323 | 0.101611538 | PLA2R1 |
| | | regulation of | | | | | | | |
| | | phospholipase | | | | | | | |
| | | A2 activity | | | | | | | |
| upregulated | GO: 2000473 | positive | 139 | 15462 | 2 | 1 | 0.017899323 | 0.101611538 | PTPRC |
| | | regulation of | | | | | | | |
| | | hematopoietic | | | | | | | |
| | | stem cell | | | | | | | |
| | | migration | | | | | | | |
| upregulated | GO: 0052565 | response to | 139 | 15462 | 2 | 1 | 0.017899323 | 0.101611538 | GUCY1A3 |
| | | defense-related | | | | | | | |
| | | host nitric oxide | | | | | | | |
| | | production | | | | | | | |
| upregulated | GO: 1902822 | regulation of late | 139 | 15462 | 2 | 1 | 0.017899323 | 0.101611538 | LRRK2 |
| | | endosome to | | | | | | | |
| | | lysosome | | | | | | | |
| | | transport | | | | | | | |
| upregulated | GO: 0071725 | response to | 139 | 15462 | 2 | 1 | 0.017899323 | 0.101611538 | TLR1 |
| | | triacyl bacterial | | | | | | | |
| | | lipopeptide | | | | | | | |
| upregulated | GO: 0090249 | regulation of cell | 139 | 15462 | 2 | 1 | 0.017899323 | 0.101611538 | NKD1 |
| | | motility involved | | | | | | | |
| | | in somitogenic | | | | | | | |
| | | axis elongation | | | | | | | |
| upregulated | GO: 2000471 | regulation of | 139 | 15462 | 2 | 1 | 0.017899323 | 0.101611538 | PTPRC |
| | | hematopoietic | | | | | | | |
| | | stem cell | | | | | | | |
| | | migration | | | | | | | |
| upregulated | GO: 0019442 | tryptophan | 139 | 15462 | 2 | 1 | 0.017899323 | 0.101611538 | TDO2 |
| | | catabolic | | | | | | | |
| | | process to | | | | | | | |
| | | acetyl-CoA | | | | | | | |
| upregulated | GO: 0071727 | cellular response | 139 | 15462 | 2 | 1 | 0.017899323 | 0.101611538 | TLR1 |
| | | to triacyl | | | | | | | |
| | | bacterial | | | | | | | |
| | | lipopeptide | | | | | | | |
| upregulated | GO: 0097018 | renal albumin | 139 | 15462 | 2 | 1 | 0.017899323 | 0.101611538 | ADIPOQ |
| | | absorption | | | | | | | |
| upregulated | GO: 0032346 | positive | 139 | 15462 | 2 | 1 | 0.017899323 | 0.101611538 | WNT4 |
| | | regulation of | | | | | | | |
| | | aldosterone | | | | | | | |
| | | metabolic | | | | | | | |
| | | process | | | | | | | |
| upregulated | GO: 0003331 | positive | 139 | 15462 | 2 | 1 | 0.017899323 | 0.101611538 | RGCC |
| | | regulation of | | | | | | | |
| | | extracellular | | | | | | | |
| | | matrix | | | | | | | |
| | | constituent | | | | | | | |
| | | secretion | | | | | | | |
| upregulated | GO: 0090247 | cell motility | 139 | 15462 | 2 | 1 | 0.017899323 | 0.101611538 | NKD1 |
| | | involved in | | | | | | | |
| | | somitogenic axis | | | | | | | |
| | | elongation | | | | | | | |
| upregulated | GO: 0003330 | regulation of | 139 | 15462 | 2 | 1 | 0.017899323 | 0.101611538 | RGCC |
| | | extracellular | | | | | | | |
| | | matrix | | | | | | | |
| | | constituent | | | | | | | |
| | | secretion | | | | | | | |
| upregulated | GO: 0035655 | interleukin-18- | 139 | 15462 | 2 | 1 | 0.017899323 | 0.101611538 | IL18R1 |
| | | mediated | | | | | | | |
| | | signaling | | | | | | | |
| | | pathway | | | | | | | |
| upregulated | GO: 0060764 | cell-cell signaling | 139 | 15462 | 2 | 1 | 0.017899323 | 0.101611538 | HOXA5 |
| | | involved in | | | | | | | |
| | | mammary gland | | | | | | | |
| | | development | | | | | | | |
| upregulated | GO: 0071812 | positive | 139 | 15462 | 2 | 1 | 0.017899323 | 0.101611538 | TNFRSF11A |
| | | regulation of | | | | | | | |
| | | fever generation | | | | | | | |
| | | by positive | | | | | | | |
| | | regulation of | | | | | | | |
| | | prostaglandin | | | | | | | |
| | | secretion | | | | | | | |
| upregulated | GO: 0035790 | platelet-derived | 139 | 15462 | 2 | 1 | 0.017899323 | 0.101611538 | ADIPOQ |
| | | growth factor | | | | | | | |
| | | receptor-alpha | | | | | | | |
| | | signaling | | | | | | | |
| | | pathway | | | | | | | |
| upregulated | GO: 0010714 | positive | 139 | 15462 | 23 | 2 | 0.017941215 | 0.101611538 | RGCC, WNT4 |
| | | regulation of | | | | | | | |
| | | collagen | | | | | | | |
| | | metabolic | | | | | | | |
| | | process | | | | | | | |
| upregulated | GO: 0043368 | positive T cell | 139 | 15462 | 23 | 2 | 0.017941215 | 0.101611538 | IL6, IRF4 |
| | | selection | | | | | | | |
| upregulated | GO: 0001960 | negative | 139 | 15462 | 23 | 2 | 0.017941215 | 0.101611538 | ADIPOQ, PTPRC |
| | | regulation of | | | | | | | |
| | | cytokine- | | | | | | | |
| | | mediated | | | | | | | |
| | | signaling | | | | | | | |
| | | pathway | | | | | | | |
| upregulated | GO: 0008211 | glucocorticoid | 139 | 15462 | 23 | 2 | 0.017941215 | 0.101611538 | WNT4, BMP2 |
| | | metabolic | | | | | | | |
| | | process | | | | | | | |
| upregulated | GO: 0046903 | secretion | 139 | 15462 | 834 | 14 | 0.01794539 | 0.101611538 | LRRK2, TLR1, CCR1, RGCC, |
| | | | | | | | | | TNFRSF11A, CD2, ITGB3, ADRA2A, |
| | | | | | | | | | LCP2, IL6, PLA2R1, CBLN4, CD36, |
| | | | | | | | | | ADIPOQ |
| upregulated | GO: 0001822 | kidney | 139 | 15462 | 231 | 6 | 0.01809629 | 0.102273006 | WNT4, LRRK2, GCNT4, CALB1, ADIPOQ, |
| | | development | | | | | | | BMP2 |
| upregulated | GO: 0070374 | positive | 139 | 15462 | 112 | 4 | 0.018412549 | 0.103691634 | TNFRSF11A, IL6, CCR1, BMP2 |
| | | regulation of | | | | | | | |
| | | ERK1 and ERK2 | | | | | | | |
| | | cascade | | | | | | | |
| upregulated | GO: 0071495 | cellular response | 139 | 15462 | 837 | 14 | 0.01845167 | 0.103691634 | NR4A1, DLX5, PDE1A, CDH13, SOS1, |
| | | to endogenous | | | | | | | BMP2, COL1A2, ADRA2A, WNT4, |
| | | stimulus | | | | | | | NAMPT, FOSL2, CD36, ADIPOQ, FOXO1 |
| upregulated | GO: 0007264 | small GTPase | 139 | 15462 | 595 | 11 | 0.018479493 | 0.103691634 | TAGAP, LRRK2, RASGRF2, CDH13, |
| | | mediated signal | | | | | | | SOS1, DEPDC7, COL1A2, ADRA2A, |
| | | transduction | | | | | | | WNT4, RAB30, DOCK8 |
| upregulated | GO: 0048878 | chemical | 139 | 15462 | 755 | 13 | 0.018485513 | 0.103691634 | LRRK2, NPTX1, STEAP4, ANGPTL4, |
| | | homeostasis | | | | | | | CCR1, CAMK2D, FABP4, PTPRC, |
| | | | | | | | | | ADRA2A, CXCL9, CALB1, ADIPOQ, |
| | | | | | | | | | FOXO1 |
| upregulated | GO: 0042110 | T cell activation | 139 | 15462 | 370 | 8 | 0.018692286 | 0.104655877 | IL18R1, IRF4, DPP4, PTPRC, CD2, |
| | | | | | | | | | WNT4, IL6, DOCK8 |
| upregulated | GO: 0018108 | peptidyl-tyrosine | 139 | 15462 | 300 | 7 | 0.01879065 | 0.104843656 | ADRA2A, EFEMP1, ITGB3, IL6, CBLC, |
| | | phosphorylation | | | | | | | CD36, ADIPOQ |
| upregulated | GO: 0001667 | ameboidal cell | 139 | 15462 | 233 | 6 | 0.018795697 | 0.104843656 | RGCC, ITGB3, NR4A1, CDH13, DPP4, |
| | | migration | | | | | | | SEMA5A |
| upregulated | GO: 0030500 | regulation of | 139 | 15462 | 63 | 3 | 0.019071475 | 0.106184593 | WNT4, CCR1, BMP2 |
| | | bone | | | | | | | |
| | | mineralization | | | | | | | |
| upregulated | GO: 1902105 | regulation of | 139 | 15462 | 171 | 5 | 0.01916013 | 0.106283825 | CD2, IL6, IRF4, CCR1, ADIPOQ |
| | | leukocyte | | | | | | | |
| | | differentiation | | | | | | | |
| upregulated | GO: 0042180 | cellular ketone | 139 | 15462 | 171 | 5 | 0.01916013 | 0.106283825 | WNT4, IL6, TDO2, ADIPOQ, BMP2 |
| | | metabolic | | | | | | | |
| | | process | | | | | | | |
| upregulated | GO: 0048519 | negative | 139 | 15462 | 3460 | 42 | 0.019211119 | 0.106323359 | FAM129A, CBLC, IRF4, SHOX2, FABP4, |
| | | regulation of | | | | | | | ADRA2A, HOXA5, BNIP3L, NPAS2, |
| | | biological | | | | | | | NKD1, DPP4, DHX34, CAMK2D, ITGB3, |
| | | process | | | | | | | RGCC, PRDM6, ERCC1, ASIC2, CXCL9, |
| | | | | | | | | | BCHE, FOXO1, NR4A3, SEMA5A, |
| | | | | | | | | | EFEMP1, MDFIC, PLA2R1, CD36, |
| | | | | | | | | | SATB2, LRRK2, BIRC3, CDH13, |
| | | | | | | | | | ANGPTL4, PTPRC, GUCY1A3, BMP2, |
| | | | | | | | | | CCR1, WNT4, IL6, TBX5, RARRES1, |
| | | | | | | | | | SKAP2, ADIPOQ |
| upregulated | GO: 0019221 | cytokine- | 139 | 15462 | 372 | 8 | 0.019238115 | 0.106323359 | TNFRSF11A, IL6, IL18R1, IRF4, CCR1, |
| | | mediated | | | | | | | ADIPOQ, PTPRC, CAMK2D |
| | | signaling | | | | | | | |
| | | pathway | | | | | | | |
| upregulated | GO: 0018212 | peptidyl-tyrosine | 139 | 15462 | 302 | 7 | 0.019409319 | 0.106868929 | ADRA2A, EFEMP1, ITGB3, IL6, CBLC, |
| | | modification | | | | | | | CD36, ADIPOQ |
| upregulated | GO: 0007176 | regulation of | 139 | 15462 | 24 | 2 | 0.019458319 | 0.106868929 | ADRA2A, CBLC |
| | | epidermal growth | | | | | | | |
| | | factor-activated | | | | | | | |
| | | receptor activity | | | | | | | |
| upregulated | GO: 0050974 | detection of | 139 | 15462 | 24 | 2 | 0.019458319 | 0.106868929 | ASIC2, PIEZO2 |
| | | mechanical | | | | | | | |
| | | stimulus involved | | | | | | | |
| | | in sensory | | | | | | | |
| | | perception | | | | | | | |
| upregulated | GO: 0030098 | lymphocyte | 139 | 15462 | 235 | 6 | 0.019513414 | 0.106868929 | CD2, WNT4, IL6, IL18R1, IRF4, PTPRC |
| | | differentiation | | | | | | | |
| upregulated | GO: 0050920 | regulation of | 139 | 15462 | 114 | 4 | 0.019514886 | 0.106868929 | IL6, CDH13, SEMA5A, CCR1 |
| | | Chemotaxis | | | | | | | |
| upregulated | GO: 0008015 | blood circulation | 139 | 15462 | 374 | 8 | 0.01979528 | 0.108206987 | ADRA2A, ELN, CELF2, ASIC2, |
| | | | | | | | | | GUCY1A3, ADIPOQ, CAMK2D, COL1A2 |
| upregulated | GO: 0006869 | lipid transport | 139 | 15462 | 236 | 6 | 0.019879202 | 0.108279291 | ATP11A, TNFRSF11A, ITGB3, PLA2R1, |
| | | | | | | | | | CD36, ADIPOQ |
| upregulated | GO: 0070167 | regulation of | 139 | 15462 | 64 | 3 | 0.01988067 | 0.108279291 | WNT4, CCR1, BMP2 |
| | | biomineral tissue | | | | | | | |
| | | development | | | | | | | |
| upregulated | GO: 0045765 | regulation of | 139 | 15462 | 173 | 5 | 0.020037503 | 0.10893577 | RGCC, IL6, HOXA5, ANGPTL4, SEMA5A |
| | | angiogenesis | | | | | | | |
| upregulated | GO: 0006915 | apoptotic | 139 | 15462 | 1637 | 23 | 0.020094062 | 0.109045716 | NR4A3, SOS1, SEMA5A, CD2, HOXA5, |
| | | process | | | | | | | FEM1B, BNIP3L, BIRC3, NR4A1, GDF6, |
| | | | | | | | | | NPTX1, RASGRF2, PDE1A, ANGPTL4, |
| | | | | | | | | | PNMA2, BMP2, RGCC, WNT4, IL6, TBX5, |
| | | | | | | | | | ASIC2, FOXO1, ADIPOQ |
| upregulated | GO: 0002520 | immune system | 139 | 15462 | 603 | 11 | 0.020177137 | 0.109298894 | IL18R1, IRF4, CCR1, PTPRC, |
| | | development | | | | | | | TNFRSF11A, CD2, WNT4, IL6, HOXA5, |
| | | | | | | | | | ERCC1, ADIPOQ |
| upregulated | GO: 0001666 | response to | 139 | 15462 | 237 | 6 | 0.020249643 | 0.109494015 | RGCC, SDC2, ANGPTL4, DPP4, |
| | | hypoxia | | | | | | | ADIPOQ, BMP2 |
| upregulated | GO: 0060341 | regulation of | 139 | 15462 | 765 | 13 | 0.020358335 | 0.109519816 | LRRK2, TLR1, IL18R1, SEMA5A, |
| | | cellular | | | | | | | CAMK2D, RGCC, CD2, ADRA2A, MDFIC, |
| | | localization | | | | | | | IL6, CXCL9, CD36, ADIPOQ |
| upregulated | GO: 0009894 | regulation of | 39 | 15462 | 765 | 13 | 0.020358335 | 0.109519816 | TAGAP, LRRK2, NKD1, PRKG1, |
| | | catabolic | | | | | | | RASGRF2, SOS1, DHX34, ADRA2A, |
| | | process | | | | | | | WNT4, PPP1R3B, ARHGAP42, DOCK8, |
| | | | | | | | | | FOXO1 |
| upregulated | GO: 0003013 | circulatory | 139 | 15462 | 376 | 8 | 0.020363898 | 0.109519816 | ADRA2A, ELN, CELF2, ASIC2, |
| | | system process | | | | | | | GUCY1A3, ADIPOQ, CAMK2D, COL1A2 |
| upregulated | GO: 0042113 | B cell activation | 139 | 15462 | 174 | 5 | 0.020485914 | 0.109978941 | IL6, ERCC1, NFATC2, SKAP2, PTPRC |
| upregulated | GO: 0001933 | negative | 139 | 15462 | 238 | 6 | 0.020624762 | 0.110311858 | FAM129A, IL6, CBLC, PTPRC, ADIPOQ, |
| | | regulation of | | | | | | | FABP4 |
| | | protein | | | | | | | |
| | | phosphorylation | | | | | | | |
| upregulated | GO: 0002009 | morphogenesis | 139 | 15462 | 377 | 8 | 0.020652538 | 0.110311858 | NKD1, SEMA5A, BMP2, WNT4, IL6, |
| | | of an epithelium | | | | | | | TBX5, FEM1B, HOXA5 |
| upregulated | GO: 0019219 | regulation of | 139 | 15462 | 4085 | 48 | 0.020658202 | 0.110311858 | TAGAP, IRF4, SHOX2, FABP4, ADRA2A, |
| | | nucleobase- | | | | | | | TNNI2, HOXA5, NFATC2, TFEC, NPAS2, |
| | | containing | | | | | | | GDF6, IL18R1, RASGRF2, HIVEP3, |
| | | compound | | | | | | | DHX34, CAMK2D, RGCC, PRDM6, BNC2, |
| | | metabolic | | | | | | | ERCC1, CXCL9, ARHGAP42, FOXO1, |
| | | process | | | | | | | NR4A3, SOS1, RFX2, RUNX1T1, |
| | | | | | | | | | EFEMP1, MDFIC, FOSL2, CD36, SATB2, |
| | | | | | | | | | DOCK8, LRRK2, ZFHX4, BIRC3, NR4A1, |
| | | | | | | | | | PRKG1, DLX5, CDH13, GUCY1A3, |
| | | | | | | | | | BMP2, TNFRSF11A, WNT4, IL6, TBX5, |
| | | | | | | | | | NAMPT, ADIPOQ |
| upregulated | GO: 0009719 | response to | 139 | 15462 | 1194 | 18 | 0.020915062 | 0.110707074 | PAPPA, NR4A3, NR4A1, DLX5, CDH13, |
| | | endogenous | | | | | | | PDE1A, SOS1, BMP2, COL1A2, ADRA2A, |
| | | stimulus | | | | | | | WNT4, IL6, NAMPT, FOSL2, BCHE, |
| | | | | | | | | | CD36, FOXO1, ADIPOQ |
| upregulated | GO: 0036293 | response to | 139 | 15462 | 239 | 6 | 0.021004583 | 0.110707074 | RGCC, SDC2, ANGPTL4, DPP4, |
| | | decreased | | | | | | | ADIPOQ, BMP2 |
| | | oxygen levels | | | | | | | |
| upregulated | GO: 0051249 | regulation of | 139 | 15462 | 307 | 7 | 0.021018312 | 0.110707074 | CD2, SLAMF7, IL6, NFATC2, IRF4, DPP4, |
| | | lymphocyte | | | | | | | PTPRC |
| | | activation | | | | | | | |
| upregulated | GO: 0032722 | positive | 139 | 15462 | 25 | 2 | 0.021027335 | 0.110707074 | IL6, ADIPOQ |
| | | regulation of | | | | | | | |
| | | chemokine | | | | | | | |
| | | production | | | | | | | |
| upregulated | GO: 0019433 | triglyceride | 139 | 15462 | 25 | 2 | 0.021027335 | 0.110707074 | PLIN1, FABP4 |
| | | catabolic | | | | | | | |
| | | process | | | | | | | |
| upregulated | GO: 0034113 | heterotypic cell- | 139 | 15462 | 25 | 2 | 0.021027335 | 0.110707074 | CD2, ADIPOQ |
| | | cell adhesion | | | | | | | |
| upregulated | GO: 0032369 | negative | 139 | 15462 | 25 | 2 | 0.021027335 | 0.110707074 | ITGB3, PLA2R1 |
| | | regulation of lipid | | | | | | | |
| | | transport | | | | | | | |
| upregulated | GO: 0060415 | muscle tissue | 139 | 15462 | 25 | 2 | 0.021027335 | 0.110707074 | SHOX2, BMP2 |
| | | morphogenesis | | | | | | | |
| upregulated | GO: 0019725 | cellular | 139 | 15462 | 607 | 11 | 0.021068161 | 0.11072776 | GLRX, NPTX1, CHRNA1, CCR1, |
| | | homeostasis | | | | | | | CAMK2D, PTPRC, ADRA2A, IL6, CXCL9, |
| | | | | | | | | | FOXO1, CALB1 |
| upregulated | GO: 0002526 | acute | 139 | 15462 | 117 | 4 | 0.021243551 | 0.111454365 | ADRA2A, TNFRSF11A, IL6, ALOX5AP |
| | | inflammatory | | | | | | | |
| | | response | | | | | | | |
| upregulated | GO: 0040013 | negative | 139 | 15462 | 176 | 5 | 0.021402354 | 0.112091561 | RGCC, WNT4, TBX5, SEMA5A, ADIPOQ |
| | | regulation of | | | | | | | |
| | | locomotion | | | | | | | |
| upregulated | GO: 0098542 | defense | 139 | 15462 | 309 | 7 | 0.021687227 | 0.113385661 | BIRC3, IL6, IGJ, BNIP3L, CXCL9, CD36, |
| | | response to | | | | | | | PTPRC |
| | | other organism | | | | | | | |
| upregulated | GO: 0002377 | immunoglobulin | 139 | 15462 | 67 | 3 | 0.022420652 | 0.115782475 | IL6, ERCC1, PTPRC |
| | | production | | | | | | | |
| upregulated | GO: 0046632 | alpha-beta T cell | 139 | 15462 | 67 | 3 | 0.022420652 | 0.115782475 | IL6, IL18R1, IRF4 |
| | | differentiation | | | | | | | |
| upregulated | GO: 0015908 | fatty acid | 139 | 15462 | 67 | 3 | 0.022420652 | 0.115782475 | TNFRSF11A, PLA2R1, CD36 |
| | | transport | | | | | | | |
| upregulated | GO: 0051707 | response to | 139 | 15462 | 613 | 11 | 0.022458889 | 0.115782475 | BIRC3, TLR1, DCLK1, GUCY1A3, |
| | | other organism | | | | | | | PTPRC, TNFRSF11A, IL6, BNIP3L, IGJ, |
| | | | | | | | | | CXCL9, CD36 |
| upregulated | GO: 0043207 | response to | 139 | 15462 | 613 | 11 | 0.022458889 | 0.115782475 | BIRC3, TLR1, DCLK1, GUCY1A3, |
| | | external biotic | | | | | | | PTPRC, TNFRSF11A, IL6, BNIP3L, IGJ, |
| | | stimulus | | | | | | | CXCL9, CD36 |
| upregulated | GO: 0072001 | renal system | 139 | 15462 | 243 | 6 | 0.022571372 | 0.115782475 | WNT4, LRRK2, GCNT4, CALB1, ADIPOQ, |
| | | development | | | | | | | BMP2 |
| upregulated | GO: 0040007 | growth | 139 | 15462 | 776 | 13 | 0.022580145 | 0.115782475 | NKD1, GDF6, CDH13, CHRNA1, DCLK1, |
| | | | | | | | | | SEMA5A, CAMK2D, BMP2, IL6, HOXA5, |
| | | | | | | | | | ERCC1, TBX5, CD36 |
| upregulated | GO: 0060997 | dendritic spine | 139 | 15462 | 26 | 2 | 0.022647222 | 0.115782475 | LRRK2, PDLIM5 |
| | | morphogenesis | | | | | | | |
| upregulated | GO: 0010862 | positive | 139 | 15462 | 26 | 2 | 0.022647222 | 0.115782475 | GDF6, BMP2 |
| | | regulation of | | | | | | | |
| | | pathway- | | | | | | | |
| | | restricted SMAD | | | | | | | |
| | | protein | | | | | | | |
| | | phosphorylation | | | | | | | |
| upregulated | GO: 0003338 | metanephros | 139 | 15462 | 26 | 2 | 0.022647222 | 0.115782475 | WNT4, CALB1 |
| | | morphogenesis | | | | | | | |
| upregulated | GO: 0043370 | regulation of | 139 | 15462 | 26 | 2 | 0.022647222 | 0.115782475 | IL6, IRF4 |
| | | CD4-positive, | | | | | | | |
| | | alpha-beta T cell | | | | | | | |
| | | differentiation | | | | | | | |
| upregulated | GO: 0003209 | cardiac atrium | 139 | 15462 | 26 | 2 | 0.022647222 | 0.115782475 | TBX5, SHOX2 |
| | | morphogenesis | | | | | | | |
| upregulated | GO: 0048512 | circadian | 139 | 15462 | 26 | 2 | 0.022647222 | 0.115782475 | NPAS2, IL6 |
| | | behavior | | | | | | | |
| upregulated | GO: 0050778 | positive | 139 | 15462 | 460 | 9 | 0.02315782 | 0.118191529 | RGCC, LCP2, BIRC3, TLR1, NFATC2, |
| | | regulation of | | | | | | | IRF4, CD36, PTPRC, FYB |
| | | immune | | | | | | | |
| | | response | | | | | | | |
| upregulated | GO: 0001952 | regulation of cell- | 139 | 15462 | 68 | 3 | 0.023304804 | 0.118588312 | WNT4, CDH13, CD36 |
| | | matrix adhesion | | | | | | | |
| upregulated | GO: 0008543 | fibroblast growth | 139 | 15462 | 180 | 5 | 0.023314597 | 0.118588312 | WNT4, NR4A1, PDE1A, SOS1, FOXO1 |
| | | factor receptor | | | | | | | |
| | | signaling | | | | | | | |
| | | pathway | | | | | | | |
| upregulated | GO: 0060485 | mesenchyme | 139 | 15462 | 181 | 5 | 0.023809371 | 0.120695813 | RGCC, WNT4, BNC2, TBX5, BMP2 |
| | | development | | | | | | | |
| upregulated | GO: 0051222 | positive | 139 | 15462 | 181 | 5 | 0.023809371 | 0.120695813 | RGCC, CD2, IL6, IL18R1, SEMA5A |
| | | regulation of | | | | | | | |
| | | protein transport | | | | | | | |
| upregulated | GO: 0030097 | hemopoiesis | 139 | 15462 | 541 | 10 | 0.024227305 | 0.121679816 | IL18R1, IRF4, CCR1, PTPRC, |
| | | | | | | | | | TNFRSF11A, CD2, WNT4, IL6, HOXA5, |
| | | | | | | | | | ADIPOQ |
| upregulated | GO: 0046461 | neutral lipid | 139 | 15462 | 27 | 2 | 0.024316952 | 0.121679816 | PLIN1, FABP4 |
| | | catabolic | | | | | | | |
| | | process | | | | | | | |
| upregulated | GO: 0003009 | skeletal muscle | 139 | 15462 | 27 | 2 | 0.024316952 | 0.121679816 | TNNI2, CHRNA1 |
| | | contraction | | | | | | | |
| upregulated | GO: 0046464 | acylglycerol | 139 | 15462 | 27 | 2 | 0.024316952 | 0.121679816 | PLIN1, FABP4 |
| | | catabolic | | | | | | | |
| | | process | | | | | | | |
| upregulated | GO: 0003197 | endocardial | 139 | 15462 | 27 | 2 | 0.024316952 | 0.121679816 | TBX5, BMP2 |
| | | cushion | | | | | | | |
| | | development | | | | | | | |
| upregulated | GO: 0060761 | negative | 139 | 15462 | 27 | 2 | 0.024316952 | 0.121679816 | ADIPOQ, PTPRC |
| | | regulation of | | | | | | | |
| | | response to | | | | | | | |
| | | cytokine stimulus | | | | | | | |
| upregulated | GO: 0010718 | positive | 139 | 15462 | 27 | 2 | 0.024316952 | 0.121679816 | RGCC, BMP2 |
| | | regulation of | | | | | | | |
| | | epithelial to | | | | | | | |
| | | mesenchymal | | | | | | | |
| | | transition | | | | | | | |
| upregulated | GO: 0050851 | antigen receptor- | 139 | 15462 | 122 | 4 | 0.024327854 | 0.121679816 | LCP2, NFATC2, PTPRC, FYB |
| | | mediated | | | | | | | |
| | | signaling | | | | | | | |
| | | pathway | | | | | | | |
| upregulated | GO: 0031344 | regulation of cell | 139 | 15462 | 317 | 7 | 0.024511172 | 0.122392725 | LRRK2, SDC2, PDLIM5, TENM3, |
| | | projection | | | | | | | SEMA5A, SHOX2, CNTN1 |
| | | organization | | | | | | | |
| upregulated | GO: 0002062 | chondrocyte | 139 | 15462 | 70 | 3 | 0.025129333 | 0.122652501 | EFEMP1, SHOX2, BMP2 |
| | | differentiation | | | | | | | |
| upregulated | GO: 0072073 | kidney | 139 | 15462 | 124 | 4 | 0.025633552 | 0.122652501 | WNT4, ADIPOQ, BMP2, CALB1 |
| | | epithelium | | | | | | | |
| | | development | | | | | | | |
| upregulated | GO: 0007622 | rhythmic | 139 | 15462 | 28 | 2 | 0.026035513 | 0.122652501 | NPAS2, IL6 |
| | | behavior | | | | | | | |
| upregulated | GO: 0032350 | regulation of | 139 | 15462 | 28 | 2 | 0.026035513 | 0.122652501 | WNT4, BMP2 |
| | | hormone | | | | | | | |
| | | metabolic | | | | | | | |
| | | process | | | | | | | |
| upregulated | GO: 0033028 | myeloid cell | 139 | 15462 | 28 | 2 | 0.026035513 | 0.122652501 | IL6, ADIPOQ |
| | | apoptotic | | | | | | | |
| | | process | | | | | | | |
| upregulated | GO: 0010677 | negative | 139 | 15462 | 28 | 2 | 0.026035513 | 0.122652501 | IL6, ADIPOQ |
| | | regulation of | | | | | | | |
| | | cellular | | | | | | | |
| | | carbohydrate | | | | | | | |
| | | metabolic | | | | | | | |
| | | process | | | | | | | |
| upregulated | GO: 0010906 | regulation of | 139 | 15462 | 71 | 3 | 0.026069689 | 0.122652501 | IL6, PPP1R3B, ADIPOQ |
| | | glucose | | | | | | | |
| | | metabolic | | | | | | | |
| | | process | | | | | | | |
| upregulated | GO: 0035270 | endocrine | 139 | 15462 | 125 | 4 | 0.026301955 | 0.122652501 | WNT4, IL6, HOXA5, FOXO1 |
| | | system | | | | | | | |
| | | development | | | | | | | |
| upregulated | GO: 0045859 | regulation of | 139 | 15462 | 710 | 12 | 0.026361918 | 0.122652501 | LRRK2, CBLC, BMP2, FABP4, PTPRC, |
| | | protein kinase | | | | | | | RGCC, TNFRSF11A, ITGB3, ADRA2A, |
| | | activity | | | | | | | MDFIC, IL6, ADIPOQ |
| upregulated | GO: 0006469 | negative | 139 | 15462 | 186 | 5 | 0.026384972 | 0.122652501 | IL6, CBLC, PTPRC, ADIPOQ, FABP4 |
| | | regulation of | | | | | | | |
| | | protein kinase | | | | | | | |
| | | activity | | | | | | | |
| upregulated | GO: 0042091 | interleukin-10 | 139 | 15462 | 3 | 1 | 0.026729335 | 0.122652501 | IRF4 |
| | | biosynthetic | | | | | | | |
| | | process | | | | | | | |
| upregulated | GO: 0048312 | intracellular | 139 | 15462 | 3 | 1 | 0.026729335 | 0.122652501 | LRRK2 |
| | | distribution of | | | | | | | |
| | | mitochondria | | | | | | | |
| upregulated | GO: 0060166 | olfactory pit | 139 | 15462 | 3 | 1 | 0.026729335 | 0.122652501 | DLX5 |
| | | development | | | | | | | |
| upregulated | GO: 0003130 | BMP signaling | 139 | 15462 | 3 | 1 | 0.026729335 | 0.122652501 | BMP2 |
| | | pathway involved | | | | | | | |
| | | in heart induction | | | | | | | |
| upregulated | GO: 0072313 | metanephric | 139 | 15462 | 3 | 1 | 0.026729335 | 0.122652501 | ADIPOQ |
| | | glomerular | | | | | | | |
| | | epithelial cell | | | | | | | |
| | | development | | | | | | | |
| upregulated | GO: 0006295 | nucleotide- | 139 | 15462 | 3 | 1 | 0.026729335 | 0.122652501 | ERCC1 |
| | | excision repair, | | | | | | | |
| | | DNA incision, 3′- | | | | | | | |
| | | to lesion | | | | | | | |
| upregulated | GO: 2000481 | positive | 139 | 15462 | 3 | 1 | 0.026729335 | 0.122652501 | ADIPOQ |
| | | regulation of | | | | | | | |
| | | cAMP- | | | | | | | |
| | | dependent | | | | | | | |
| | | protein kinase | | | | | | | |
| | | activity | | | | | | | |
| upregulated | GO: 0072249 | metanephric | 139 | 15462 | 3 | 1 | 0.026729335 | 0.122652501 | ADIPOQ |
| | | glomerular | | | | | | | |
| | | visceral epithelial | | | | | | | |
| | | cell development | | | | | | | |
| upregulated | GO: 0072244 | metanephric | 139 | 15462 | 3 | 1 | 0.026729335 | 0.122652501 | ADIPOQ |
| | | glomerular | | | | | | | |
| | | epithelium | | | | | | | |
| | | development | | | | | | | |
| upregulated | GO: 0061209 | cell proliferation | 139 | 15462 | 3 | 1 | 0.026729335 | 0.122652501 | BMP2 |
| | | involved in | | | | | | | |
| | | mesonephros | | | | | | | |
| | | development | | | | | | | |
| upregulated | GO: 2000623 | negative | 139 | 15462 | 3 | 1 | 0.026729335 | 0.122652501 | DHX34 |
| | | regulation of | | | | | | | |
| | | nuclear- | | | | | | | |
| | | transcribed | | | | | | | |
| | | mRNA catabolic | | | | | | | |
| | | process, | | | | | | | |
| | | nonsense- | | | | | | | |
| | | mediated decay | | | | | | | |
| upregulated | GO: 0045324 | late endosome to | 139 | 15462 | 3 | 1 | 0.026729335 | 0.122652501 | LRRK2 |
| | | vacuole transport | | | | | | | |
| upregulated | GO: 2000726 | negative | 139 | 15462 | 3 | 1 | 0.026729335 | 0.122652501 | BMP2 |
| | | regulation of | | | | | | | |
| | | cardiac muscle | | | | | | | |
| | | cell | | | | | | | |
| | | differentiation | | | | | | | |
| upregulated | GO: 0071883 | activation of | 139 | 15462 | 3 | 1 | 0.026729335 | 0.122652501 | ADRA2A |
| | | MAPK activity by | | | | | | | |
| | | adrenergic | | | | | | | |
| | | receptor | | | | | | | |
| | | signaling | | | | | | | |
| | | pathway | | | | | | | |
| upregulated | GO: 0045404 | positive | 139 | 15462 | 3 | 1 | 0.026729335 | 0.122652501 | IRF4 |
| | | regulation of | | | | | | | |
| | | interleukin-4 | | | | | | | |
| | | biosynthetic | | | | | | | |
| | | process | | | | | | | |
| upregulated | GO: 0060161 | positive | 139 | 15462 | 3 | 1 | 0.026729335 | 0.122652501 | LRRK2 |
| | | regulation of | | | | | | | |
| | | dopamine | | | | | | | |
| | | receptor | | | | | | | |
| | | signaling | | | | | | | |
| | | pathway | | | | | | | |
| upregulated | GO: 0090270 | regulation of | 139 | 15462 | 3 | 1 | 0.026729335 | 0.122652501 | RGCC |
| | | fibroblast growth | | | | | | | |
| | | factor production | | | | | | | |
| upregulated | GO: 0061184 | positive | 139 | 15462 | 3 | 1 | 0.026729335 | 0.122652501 | WNT4 |
| | | regulation of | | | | | | | |
| | | dermatome | | | | | | | |
| | | development | | | | | | | |
| upregulated | GO: 0000720 | pyrimidine dimer | 139 | 15462 | 3 | 1 | 0.026729335 | 0.122652501 | ERCC1 |
| | | repair by | | | | | | | |
| | | nucleotide- | | | | | | | |
| | | excision repair | | | | | | | |
| upregulated | GO: 0035694 | mitochondrial | 139 | 15462 | 3 | 1 | 0.026729335 | 0.122652501 | BNIP3L |
| | | protein catabolic | | | | | | | |
| | | process | | | | | | | |
| upregulated | GO: 0072138 | mesenchymal | 139 | 15462 | 3 | 1 | 0.026729335 | 0.122652501 | BMP2 |
| | | cell proliferation | | | | | | | |
| | | involved in | | | | | | | |
| | | ureteric bud | | | | | | | |
| | | development | | | | | | | |
| upregulated | GO: 0072248 | metanephric | 139 | 15462 | 3 | 1 | 0.026729335 | 0.122652501 | ADIPOQ |
| | | glomerular | | | | | | | |
| | | visceral epithelial | | | | | | | |
| | | cell | | | | | | | |
| | | differentiation | | | | | | | |
| upregulated | GO: 0003133 | endodermal- | 139 | 15462 | 3 | 1 | 0.026729335 | 0.122652501 | BMP2 |
| | | mesodermal cell | | | | | | | |
| | | signaling | | | | | | | |
| upregulated | GO: 0071351 | cellular response | 139 | 15462 | 3 | 1 | 0.026729335 | 0.122652501 | IL18R1 |
| | | to interleukin-18 | | | | | | | |
| upregulated | GO: 2000622 | regulation of | 139 | 15462 | 3 | 1 | 0.026729335 | 0.122652501 | DHX34 |
| | | nuclear- | | | | | | | |
| | | transcribed | | | | | | | |
| | | mRNA catabolic | | | | | | | |
| | | process, | | | | | | | |
| | | nonsense- | | | | | | | |
| | | mediated decay | | | | | | | |
| upregulated | GO: 2000300 | regulation of | 139 | 15462 | 3 | 1 | 0.026729335 | 0.122652501 | LRRK2 |
| | | synaptic vesicle | | | | | | | |
| | | exocytosis | | | | | | | |
| upregulated | GO: 0035441 | cell migration | 139 | 15462 | 3 | 1 | 0.026729335 | 0.122652501 | TBX5 |
| | | involved in | | | | | | | |
| | | vasculogenesis | | | | | | | |
| upregulated | GO: 2000366 | positive | 139 | 15462 | 3 | 1 | 0.026729335 | 0.122652501 | IL6 |
| | | regulation of | | | | | | | |
| | | STAT protein | | | | | | | |
| | | import into | | | | | | | |
| | | nucleus | | | | | | | |
| upregulated | GO: 0090269 | fibroblast growth | 139 | 15462 | 3 | 1 | 0.026729335 | 0.122652501 | RGCC |
| | | factor production | | | | | | | |
| upregulated | GO: 0045074 | regulation of | 139 | 15462 | 3 | 1 | 0.026729335 | 0.122652501 | IRF4 |
| | | interleukin-10 | | | | | | | |
| | | biosynthetic | | | | | | | |
| | | process | | | | | | | |
| upregulated | GO: 0060435 | bronchiole | 139 | 15462 | 3 | 1 | 0.026729335 | 0.122652501 | HOXA5 |
| | | development | | | | | | | |
| upregulated | GO: 0002378 | immunoglobulin | 139 | 15462 | 3 | 1 | 0.026729335 | 0.122652501 | PTPRC |
| | | biosynthetic | | | | | | | |
| | | process | | | | | | | |
| upregulated | GO: 0060574 | intestinal | 139 | 15462 | 3 | 1 | 0.026729335 | 0.122652501 | HOXA5 |
| | | epithelial cell | | | | | | | |
| | | maturation | | | | | | | |
| upregulated | GO: 2000364 | regulation of | 139 | 15462 | 3 | 1 | 0.026729335 | 0.122652501 | IL6 |
| | | STAT protein | | | | | | | |
| | | import into | | | | | | | |
| | | nucleus | | | | | | | |
| upregulated | GO: 0070340 | detection of | 139 | 15462 | 3 | 1 | 0.026729335 | 0.122652501 | TLR1 |
| | | bacterial | | | | | | | |
| | | lipopeptide | | | | | | | |
| upregulated | GO: 0003134 | endodermal- | 139 | 15462 | 3 | 1 | 0.026729335 | 0.122652501 | BMP2 |
| | | mesodermal cell | | | | | | | |
| | | signaling | | | | | | | |
| | | involved in heart | | | | | | | |
| | | induction | | | | | | | |
| upregulated | GO: 0048743 | positive | 139 | 15462 | 3 | 1 | 0.026729335 | 0.122652501 | SHOX2 |
| | | regulation of | | | | | | | |
| | | skeletal muscle | | | | | | | |
| | | fiber | | | | | | | |
| | | development | | | | | | | |
| upregulated | GO: 0006296 | nucleotide- | 139 | 15462 | 3 | 1 | 0.026729335 | 0.122652501 | ERCC1 |
| | | excision repair, | | | | | | | |
| | | DNA incision, 5′- | | | | | | | |
| | | to lesion | | | | | | | |
| upregulated | GO: 0072312 | metanephric | 139 | 15462 | 3 | 1 | 0.026729335 | 0.122652501 | ADIPOQ |
| | | glomerular | | | | | | | |
| | | epithelial cell | | | | | | | |
| | | differentiation | | | | | | | |
| upregulated | GO: 0015015 | heparan sulfate | 139 | 15462 | 3 | 1 | 0.026729335 | 0.122652501 | HS3ST3B1 |
| | | proteoglycan | | | | | | | |
| | | biosynthetic | | | | | | | |
| | | process, | | | | | | | |
| | | enzymatic | | | | | | | |
| | | modification | | | | | | | |
| upregulated | GO: 0002540 | leukotriene | 139 | 15462 | 3 | 1 | 0.026729335 | 0.122652501 | ALOX5AP |
| | | production | | | | | | | |
| | | involved in | | | | | | | |
| | | inflammatory | | | | | | | |
| | | response | | | | | | | |
| upregulated | GO: 0071639 | positive | 139 | 15462 | 3 | 1 | 0.026729335 | 0.122652501 | ADIPOQ |
| | | regulation of | | | | | | | |
| | | monocyte | | | | | | | |
| | | chemotactic | | | | | | | |
| | | protein-1 | | | | | | | |
| | | production | | | | | | | |
| upregulated | GO: 0002538 | arachidonic acid | 139 | 15462 | 3 | 1 | 0.026729335 | 0.122652501 | ALOX5AP |
| | | metabolite | | | | | | | |
| | | production | | | | | | | |
| | | involved in | | | | | | | |
| | | inflammatory | | | | | | | |
| | | response | | | | | | | |
| upregulated | GO: 0010035 | response to | 139 | 15462 | 323 | 7 | 0.026788702 | 0.122737242 | NR4A3, IL6, ALOX5AP, CD36, FOXO1, |
| | | inorganic | | | | | | | FABP4, CAMK2D |
| | | substance | | | | | | | |
| upregulated | GO: 0090317 | negative | 139 | 15462 | 72 | 3 | 0.027028753 | 0.123648303 | MDFIC, CD36, ADIPOQ |
| | | regulation of | | | | | | | |
| | | intracellular | | | | | | | |
| | | protein transport | | | | | | | |
| upregulated | GO: 0031347 | regulation of | 139 | 15462 | 473 | 9 | 0.027079118 | 0.123690156 | TNFRSF11A, BIRC3, IL6, TLR1, |
| | | defense | | | | | | | ALOX5AP, IRF4, CD36, ADIPOQ, FABP4 |
| | | response | | | | | | | |
| upregulated | GO: 0017038 | protein import | 139 | 15462 | 254 | 6 | 0.027281991 | 0.124427441 | IL6, MDFIC, IL18R1, SEMA5A, CD36, |
| | | | | | | | | | FYB |
| upregulated | GO: 0044710 | single-organism | 139 | 15462 | 5094 | 57 | 0.027609815 | 0.125463929 | TAGAP, GPC6, CBLC, IRF4, HS3ST3B1, |
| | | metabolic | | | | | | | FABP4, ADRA2A, COL12A1, RAB30, IGJ, |
| | | process | | | | | | | NPAS2, NKD1, GLRX, RASGRF2, |
| | | | | | | | | | GALNT5, AOX1, COL1A2, CAMK2D, |
| | | | | | | | | | RGCC, PRDM6, FMO4, ERCC1, CXCL9, |
| | | | | | | | | | ADH1B, BCHE, SLC22A3, FOXO1, |
| | | | | | | | | | ARHGAP42, CRLS1, STEAP4, PRELP, |
| | | | | | | | | | SOS1, MDFIC, PPP1R3B, ALOX5AP, |
| | | | | | | | | | CD36, MMP1, DOCK8, LRRK2, PRKG1, |
| | | | | | | | | | GCNT4, SDC2, PDE1A, PLIN1, |
| | | | | | | | | | ANGPTL4, GUCY1A3, CCR1, BMP2, |
| | | | | | | | | | TNFRSF11A, GALNT14, WNT4, IL6, |
| | | | | | | | | | CHSY3, NAMPT, SLC7A5, TDO2, |
| | | | | | | | | | ADIPOQ |
| upregulated | GO: 0072089 | stem cell | 139 | 15462 | 127 | 4 | 0.027670013 | 0.125463929 | LRRK2, SHOX2, PTPRC, BMP2 |
| | | proliferation | | | | | | | |
| upregulated | GO: 0043277 | apoptotic cell | 139 | 15462 | 29 | 2 | 0.027801904 | 0.125463929 | MEGF10, CD36 |
| | | clearance | | | | | | | |
| upregulated | GO: 0003230 | cardiac atrium | 139 | 15462 | 29 | 2 | 0.027801904 | 0.125463929 | TBX5, SHOX2 |
| | | development | | | | | | | |
| upregulated | GO: 0051148 | negative | 139 | 15462 | 29 | 2 | 0.027801904 | 0.125463929 | PRDM6, BMP2 |
| | | regulation of | | | | | | | |
| | | muscle cell | | | | | | | |
| | | differentiation | | | | | | | |
| upregulated | GO: 0032309 | icosanoid | 139 | 15462 | 29 | 2 | 0.027801904 | 0.125463929 | TNFRSF11A, PLA2R1 |
| | | secretion | | | | | | | |
| upregulated | GO: 2000514 | regulation of | 139 | 15462 | 29 | 2 | 0.027801904 | 0.125463929 | IL6, IRF4 |
| | | CD4-positive, | | | | | | | |
| | | alpha-beta T cell | | | | | | | |
| | | activation | | | | | | | |
| upregulated | GO: 0071248 | cellular response | 139 | 15462 | 73 | 3 | 0.028006501 | 0.12619746 | ALOX5AP, FABP4, CAMK2D |
| | | to metal ion | | | | | | | |
| upregulated | GO: 0071900 | regulation of | 139 | 15462 | 400 | 8 | 0.028121756 | 0.126526823 | LRRK2, CBLC, BMP2, TNFRSF11A, |
| | | protein | | | | | | | ADRA2A, RGCC, MDFIC, ADIPOQ |
| | | serine/threonine | | | | | | | |
| | | kinase activity | | | | | | | |
| upregulated | GO: 0006936 | muscle | 139 | 15462 | 256 | 6 | 0.028203395 | 0.126704173 | ADRA2A, TNNI2, CHRNA1, LMOD1, |
| | | contraction | | | | | | | CAMK2D, GUCY1A3 |
| upregulated | GO: 2000021 | regulation of ion | 139 | 15462 | 128 | 4 | 0.02836972 | 0.12707094 | LRRK2, CXCL9, PTPRC, CAMK2D |
| | | homeostasis | | | | | | | |
| upregulated | GO: 0007623 | circadian rhythm | 139 | 15462 | 128 | 4 | 0.02836972 | 0.12707094 | TNFRSF11A, NPAS2, IL6, ADIPOQ |
| upregulated | GO: 0051171 | regulation of | 139 | 15462 | 4162 | 48 | 0.028514832 | 0.127530568 | TAGAP, SHOX2, IRF4, FABP4, ADRA2A, |
| | | nitrogen | | | | | | | TNNI2, HOXA5, NFATC2, TFEC, NPAS2, |
| | | compound | | | | | | | GDF6, RASGRF2, IL18R1, HIVEP3, |
| | | metabolic | | | | | | | CAMK2D, DHX34, RGCC, PRDM6, BNC2, |
| | | process | | | | | | | ERCC1, CXCL9, FOXO1, ARHGAP42, |
| | | | | | | | | | NR4A3, SOS1, RFX2, RUNX1T1, |
| | | | | | | | | | EFEMP1, MDFIC, FOSL2, CD36, SATB2, |
| | | | | | | | | | DOCK8, LRRK2, ZFHX4, BIRC3, NR4A1, |
| | | | | | | | | | PRKG1, DLX5, CDH13, GUCY1A3, |
| | | | | | | | | | BMP2, TNFRSF11A, WNT4, IL6, TBX5, |
| | | | | | | | | | NAMPT, ADIPOQ |
| upregulated | GO: 0042307 | positive | 139 | 15462 | 74 | 3 | 0.029002906 | 0.129095881 | IL6, IL18R1, SEMA5A |
| | | regulation of | | | | | | | |
| | | protein import | | | | | | | |
| | | into nucleus | | | | | | | |
| upregulated | GO: 0045666 | positive | 139 | 15462 | 74 | 3 | 0.029002906 | 0.129095881 | IL6, GDF6, BMP2 |
| | | regulation of | | | | | | | |
| | | neuron | | | | | | | |
| | | differentiation | | | | | | | |
| upregulated | GO: 0030574 | collagen | 139 | 15462 | 74 | 3 | 0.029002906 | 0.129095881 | COL12A1, COL1A2, MMP1 |
| | | catabolic | | | | | | | |
| | | process | | | | | | | |
| upregulated | GO: 0045860 | positive | 139 | 15462 | 479 | 9 | 0.029039124 | 0.129095881 | LRRK2, BMP2, PTPRC, RGCC, ITGB3, |
| | | regulation of | | | | | | | TNFRSF11A, ADRA2A, MDFIC, ADIPOQ |
| | | protein kinase | | | | | | | |
| | | activity | | | | | | | |
| upregulated | GO: 0000187 | activation of | 139 | 15462 | 129 | 4 | 0.029079912 | 0.129095881 | ADRA2A, LRRK2, MDFIC, BMP2 |
| | | MAPK activity | | | | | | | |
| upregulated | GO: 0051235 | maintenance of | 139 | 15462 | 258 | 6 | 0.029145179 | 0.129194509 | ITGB3, IL6, CXCL9, CD36, PTPRC, |
| | | location | | | | | | | CAMK2D |
| upregulated | GO: 0044253 | positive | 139 | 15462 | 30 | 2 | 0.02961514 | 0.130891066 | RGCC, WNT4 |
| | | regulation of | | | | | | | |
| | | multicellular | | | | | | | |
| | | organismal | | | | | | | |
| | | metabolic | | | | | | | |
| | | process | | | | | | | |
| upregulated | GO: 0050710 | negative | 139 | 15462 | 30 | 2 | 0.02961514 | 0.130891066 | RGCC, IL6 |
| | | regulation of | | | | | | | |
| | | cytokine | | | | | | | |
| | | secretion | | | | | | | |
| upregulated | GO: 0043406 | positive | 139 | 15462 | 192 | 5 | 0.029703002 | 0.131086338 | ADRA2A, TNFRSF11A, LRRK2, MDFIC, |
| | | regulation of | | | | | | | BMP2 |
| | | MAP kinase | | | | | | | |
| | | activity | | | | | | | |
| upregulated | GO: 0051282 | regulation of | 139 | 15462 | 75 | 3 | 0.030017939 | 0.131317545 | CXCL9, PTPRC, CAMK2D |
| | | sequestering of | | | | | | | |
| | | calcium ion | | | | | | | |
| upregulated | GO: 0050864 | regulation of B | 139 | 15462 | 75 | 3 | 0.030017939 | 0.131317545 | IL6, NFATC2, PTPRC |
| | | cell activation | | | | | | | |
| upregulated | GO: 0051209 | release of | 139 | 15462 | 75 | 3 | 0.030017939 | 0.131317545 | CXCL9, PTPRC, CAMK2D |
| | | sequestered | | | | | | | |
| | | calcium ion into | | | | | | | |
| | | cytosol | | | | | | | |
| upregulated | GO: 0051283 | negative | 139 | 15462 | 75 | 3 | 0.030017939 | 0.131317545 | CXCL9, PTPRC, CAMK2D |
| | | regulation of | | | | | | | |
| | | sequestering of | | | | | | | |
| | | calcium ion | | | | | | | |
| upregulated | GO: 0046824 | positive | 139 | 15462 | 75 | 3 | 0.030017939 | 0.131317545 | IL6, IL18R1, SEMA5A |
| | | regulation of | | | | | | | |
| | | nucleocytoplasmic | | | | | | | |
| | | transport | | | | | | | |
| upregulated | GO: 0007569 | cell aging | 139 | 15462 | 75 | 3 | 0.030017939 | 0.131317545 | PLA2R1, ERCC1, PRELP |
| upregulated | GO: 0009607 | response to | 139 | 15462 | 643 | 11 | 0.030443846 | 0.132986873 | BIRC3, TLR1, DCLK1, GUCY1A3, |
| | | biotic stimulus | | | | | | | PTPRC, TNFRSF11A, IL6, BNIP3L, IGJ, |
| | | | | | | | | | CXCL9, CD36 |
| upregulated | GO: 0051924 | regulation of | 139 | 15462 | 131 | 4 | 0.03053184 | 0.1331774 | ADRA2A, CXCL9, CCR1, CAMK2D |
| | | calcium ion | | | | | | | |
| | | transport | | | | | | | |
| upregulated | GO: 0090257 | regulation of | 139 | 15462 | 132 | 4 | 0.031273616 | 0.134550171 | ADRA2A, FBXO32, CAMK2D, GUCY1A3 |
| | | muscle system | | | | | | | |
| | | process | | | | | | | |
| upregulated | GO: 0032270 | positive | 139 | 15462 | 898 | 14 | 0.031293759 | 0.134550171 | FAM129A, LRRK2, BIRC3, GDF6, BMP2, |
| | | regulation of | | | | | | | PTPRC, RGCC, TNFRSF11A, ADRA2A, |
| | | cellular protein | | | | | | | ITGB3, IL6, MDFIC, CD36, ADIPOQ |
| | | metabolic | | | | | | | |
| | | process | | | | | | | |
| upregulated | GO: 0007610 | behavior | 139 | 15462 | 565 | 10 | 0.031365402 | 0.134550171 | NR4A3, NPAS2, LRRK2, CDH13, GCNT4, |
| | | | | | | | | | SEMA5A, CCR1, IL6, BCHE, CALB1 |
| upregulated | GO: 0051046 | regulation of | 139 | 15462 | 486 | 9 | 0.031449748 | 0.134550171 | LRRK2, TLR1, CD2, TNFRSF11A, |
| | | secretion | | | | | | | ADRA2A, RGCC, IL6, PLA2R1, ADIPOQ |
| upregulated | GO: 1901571 | fatty acid | 139 | 15462 | 31 | 2 | 0.031474249 | 0.134550171 | TNFRSF11A, PLA2R1 |
| | | derivative | | | | | | | |
| | | transport | | | | | | | |
| upregulated | GO: 0071829 | plasma | 139 | 15462 | 31 | 2 | 0.031474249 | 0.134550171 | CD36, ADIPOQ |
| | | lipoprotein | | | | | | | |
| | | particle | | | | | | | |
| | | disassembly | | | | | | | |
| upregulated | GO: 0034381 | plasma | 139 | 15462 | 31 | 2 | 0.031474249 | 0.134550171 | CD36, ADIPOQ |
| | | lipoprotein | | | | | | | |
| | | particle | | | | | | | |
| | | clearance | | | | | | | |
| upregulated | GO: 0097061 | dendritic spine | 139 | 15462 | 31 | 2 | 0.031474249 | 0.134550171 | LRRK2, PDLIM5 |
| | | organization | | | | | | | |
| upregulated | GO: 0032987 | protein-lipid | 139 | 15462 | 31 | 2 | 0.031474249 | 0.134550171 | CD36, ADIPOQ |
| | | complex | | | | | | | |
| | | disassembly | | | | | | | |
| upregulated | GO: 0051055 | negative | 139 | 15462 | 31 | 2 | 0.031474249 | 0.134550171 | WNT4, BMP2 |
| | | regulation of lipid | | | | | | | |
| | | biosynthetic | | | | | | | |
| | | process | | | | | | | |
| upregulated | GO: 2000278 | regulation of | 139 | 15462 | 31 | 2 | 0.031474249 | 0.134550171 | RGCC, ADIPOQ |
| | | DNA biosynthetic | | | | | | | |
| | | process | | | | | | | |
| upregulated | GO: 0003254 | regulation of | 139 | 15462 | 31 | 2 | 0.031474249 | 0.134550171 | LRRK2, CAMK2D |
| | | membrane | | | | | | | |
| | | depolarization | | | | | | | |
| upregulated | GO: 0048644 | muscle organ | 139 | 15462 | 31 | 2 | 0.031474249 | 0.134550171 | SHOX2, BMP2 |
| | | morphogenesis | | | | | | | |
| upregulated | GO: 0071715 | icosanoid | 139 | 15462 | 31 | 2 | 0.031474249 | 0.134550171 | TNFRSF11A, PLA2R1 |
| | | transport | | | | | | | |
| upregulated | GO: 0034504 | protein | 139 | 15462 | 263 | 6 | 0.031589791 | 0.134852008 | MDFIC, IL6, IL18R1, SEMA5A, CD36, |
| | | localization to | | | | | | | FYB |
| | | nucleus | | | | | | | |
| upregulated | GO: 0045087 | innate immune | 139 | 15462 | 814 | 13 | 0.031665011 | 0.134981104 | SLAMF7, BIRC3, NR4A1, TLR1, PDE1A, |
| | | response | | | | | | | SOS1, IRF4, CAMK2D, LCP2, IGJ, |
| | | | | | | | | | NFATC2, CD36, FOXO1 |
| upregulated | GO: 0048534 | hematopoietic or | 139 | 15462 | 567 | 10 | 0.032020907 | 0.135694827 | IL18R1, IRF4, CCR1, PTPRC, |
| | | lymphoid organ | | | | | | | TNFRSF11A, CD2, WNT4, IL6, HOXA5, |
| | | development | | | | | | | ADIPOQ |
| upregulated | GO: 0014031 | mesenchymal | 139 | 15462 | 133 | 4 | 0.032025959 | 0.135694827 | RGCC, WNT4, TBX5, BMP2 |
| | | cell development | | | | | | | |
| upregulated | GO: 0010959 | regulation of | 139 | 15462 | 196 | 5 | 0.032055193 | 0.135694827 | ADRA2A, CXCL9, CCR1, CNTN1, |
| | | metal ion | | | | | | | CAMK2D |
| | | transport | | | | | | | |
| upregulated | GO: 0009615 | response to virus | 139 | 15462 | 264 | 6 | 0.03209431 | 0.135694827 | BIRC3, IL6, BNIP3L, CXCL9, DCLK1, |
| | | | | | | | | | PTPRC |
| upregulated | GO: 0060021 | palate | 139 | 15462 | 77 | 3 | 0.032103741 | 0.135694827 | BNC2, DLX5, SATB2 |
| | | development | | | | | | | |
| upregulated | GO: 2000736 | regulation of | 139 | 15462 | 77 | 3 | 0.032103741 | 0.135694827 | RGCC, TBX5, BMP2 |
| | | stem cell | | | | | | | |
| | | differentiation | | | | | | | |
| upregulated | GO: 0010720 | positive | 139 | 15462 | 197 | 5 | 0.032660959 | 0.136159913 | RGCC, SHOX2, SEMA5A, ADIPOQ, |
| | | regulation of cell | | | | | | | BMP2 |
| | | development | | | | | | | |
| upregulated | GO: 0072593 | reactive oxygen | 139 | 15462 | 134 | 4 | 0.032788885 | 0.136159913 | BIRC3, PLA2R1, CD36, AOX1 |
| | | species | | | | | | | |
| | | metabolic | | | | | | | |
| | | process | | | | | | | |
| upregulated | GO: 0001656 | metanephros | 139 | 15462 | 78 | 3 | 0.033174427 | 0.136159913 | WNT4, ADIPOQ, CALB1 |
| | | development | | | | | | | |
| upregulated | GO: 0051208 | sequestering of | 139 | 15462 | 78 | 3 | 0.033174427 | 0.136159913 | CXCL9, PTPRC, CAMK2D |
| | | calcium ion | | | | | | | |
| upregulated | GO: 0044344 | cellular response | 139 | 15462 | 198 | 5 | 0.033273853 | 0.136159913 | WNT4, NR4A1, PDE1A, SOS1, FOXO1 |
| | | to fibroblast | | | | | | | |
| | | growth factor | | | | | | | |
| | | stimulus | | | | | | | |
| upregulated | GO: 0030501 | positive | 139 | 15462 | 32 | 2 | 0.033378272 | 0.136159913 | WNT4, BMP2 |
| | | regulation of | | | | | | | |
| | | bone | | | | | | | |
| | | mineralization | | | | | | | |
| upregulated | GO: 1901019 | regulation of | 139 | 15462 | 32 | 2 | 0.033378272 | 0.136159913 | ADRA2A, CAMK2D |
| | | calcium ion | | | | | | | |
| | | transmembrane | | | | | | | |
| | | transporter | | | | | | | |
| | | activity | | | | | | | |
| upregulated | GO: 0050873 | brown fat cell | 139 | 15462 | 32 | 2 | 0.033378272 | 0.136159913 | FABP4, ADIPOQ |
| | | differentiation | | | | | | | |
| upregulated | GO: 1903169 | regulation of | 139 | 15462 | 32 | 2 | 0.033378272 | 0.136159913 | ADRA2A, CAMK2D |
| | | calcium ion | | | | | | | |
| | | transmembrane | | | | | | | |
| | | transport | | | | | | | |
| upregulated | GO: 0035115 | embryonic | 139 | 15462 | 32 | 2 | 0.033378272 | 0.136159913 | TBX5, SHOX2 |
| | | forelimb | | | | | | | |
| | | morphogenesis | | | | | | | |
| upregulated | GO: 0070169 | positive | 139 | 15462 | 32 | 2 | 0.033378272 | 0.136159913 | WNT4, BMP2 |
| | | regulation of | | | | | | | |
| | | biomineral tissue | | | | | | | |
| | | development | | | | | | | |
| upregulated | GO: 0060998 | regulation of | 139 | 15462 | 32 | 2 | 0.033378272 | 0.136159913 | LRRK2, PDLIM5 |
| | | dendritic spine | | | | | | | |
| | | development | | | | | | | |
| upregulated | GO: 1900542 | regulation of | 139 | 15462 | 653 | 11 | 0.033510327 | 0.136159913 | TAGAP, LRRK2, PRKG1, RASGRF2, |
| | | purine nucleotide | | | | | | | SOS1, GUCY1A3, ADRA2A, WNT4, |
| | | metabolic | | | | | | | CXCL9, ARHGAP42, DOCK8 |
| | | process | | | | | | | |
| upregulated | GO: 0010565 | regulation of | 139 | 15462 | 135 | 4 | 0.03356241 | 0.136159913 | WNT4, IL6, ADIPOQ, BMP2 |
| | | cellular ketone | | | | | | | |
| | | metabolic | | | | | | | |
| | | process | | | | | | | |
| upregulated | GO: 0033673 | negative | 139 | 15462 | 199 | 5 | 0.033893892 | 0.136159913 | IL6, CBLC, PTPRC, ADIPOQ, FABP4 |
| | | regulation of | | | | | | | |
| | | kinase activity | | | | | | | |
| upregulated | GO: 0006493 | protein O-linked | 139 | 15462 | 79 | 3 | 0.034263575 | 0.136159913 | GALNT14, GCNT4, GALNT5 |
| | | glycosylation | | | | | | | |
| upregulated | GO: 0032675 | regulation of | 139 | 15462 | 79 | 3 | 0.034263575 | 0.136159913 | IL6, TLR1, CD36 |
| | | interleukin-6 | | | | | | | |
| | | production | | | | | | | |
| upregulated | GO: 0010811 | positive | 139 | 15462 | 79 | 3 | 0.034263575 | 0.136159913 | WNT4, CDH13, CD36 |
| | | regulation of cell- | | | | | | | |
| | | substrate | | | | | | | |
| | | adhesion | | | | | | | |
| upregulated | GO: 0006140 | regulation of | 139 | 15462 | 656 | 11 | 0.034471728 | 0.136159913 | TAGAP, LRRK2, PRKG1, RASGRF2, |
| | | nucleotide | | | | | | | SOS1, GUCY1A3, ADRA2A, WNT4, |
| | | metabolic | | | | | | | CXCL9, ARHGAP42, DOCK8 |
| | | process | | | | | | | |
| upregulated | GO: 0002694 | regulation of | 139 | 15462 | 341 | 7 | 0.034478745 | 0.136159913 | CD2, SLAMF7, IL6, NFATC2, IRF4, DPP4, |
| | | leukocyte | | | | | | | PTPRC |
| | | activation | | | | | | | |
| upregulated | GO: 0043087 | regulation of | 139 | 15462 | 417 | 8 | 0.034726426 | 0.136159913 | TAGAP, LRRK2, PRKG1, RASGRF2, |
| | | GTPase activity | | | | | | | SOS1, WNT4, ARHGAP42, DOCK8 |
| upregulated | GO: 0032388 | positive | 139 | 15462 | 137 | 4 | 0.035141307 | 0.136159913 | IL6, CXCL9, IL18R1, SEMA5A |
| | | regulation of | | | | | | | |
| | | intracellular | | | | | | | |
| | | transport | | | | | | | |
| upregulated | GO: 0033124 | regulation of | 139 | 15462 | 418 | 8 | 0.035145116 | 0.136159913 | TAGAP, LRRK2, PRKG1, RASGRF2, |
| | | GTP catabolic | | | | | | | SOS1, WNT4, ARHGAP42, DOCK8 |
| | | process | | | | | | | |
| upregulated | GO: 0071774 | response to | 139 | 15462 | 201 | 5 | 0.035155478 | 0.136159913 | WNT4, NR4A1, PDE1A, SOS1, FOXO1 |
| | | fibroblast growth | | | | | | | |
| | | factor | | | | | | | |
| upregulated | GO: 0071277 | cellular response | 139 | 15462 | 33 | 2 | 0.035326265 | 0.136159913 | ALOX5AP, CAMK2D |
| | | to calcium ion | | | | | | | |
| upregulated | GO: 0060325 | face | 139 | 15462 | 33 | 2 | 0.035326265 | 0.136159913 | CRISPLD2, DLX5 |
| | | morphogenesis | | | | | | | |
| upregulated | GO: 0044243 | multicellular | 139 | 15462 | 80 | 3 | 0.035371133 | 0.136159913 | COL12A1, COL1A2, MMP1 |
| | | organismal | | | | | | | |
| | | catabolic | | | | | | | |
| | | process | | | | | | | |
| upregulated | GO: 0072091 | regulation of | 139 | 15462 | 80 | 3 | 0.035371133 | 0.136159913 | LRRK2, SHOX2, PTPRC |
| | | stem cell | | | | | | | |
| | | proliferation | | | | | | | |
| upregulated | GO: 0051291 | protein | 139 | 15462 | 80 | 3 | 0.035371133 | 0.136159913 | BIRC3, COL1A2, ADIPOQ |
| | | heterooligomerization | | | | | | | |
| upregulated | GO: 0030900 | forebrain | 139 | 15462 | 343 | 7 | 0.035415114 | 0.136159913 | WNT4, NR4A3, LRRK2, PRKG1, DCLK1, |
| | | development | | | | | | | SEMA5A, BMP2 |
| upregulated | GO: 0003164 | His-Purkinje | 139 | 15462 | 4 | 1 | 0.035480524 | 0.136159913 | TBX5 |
| | | system | | | | | | | |
| | | development | | | | | | | |
| upregulated | GO: 1901311 | regulation of | 139 | 15462 | 4 | 1 | 0.035480524 | 0.136159913 | RGCC |
| | | gene expression | | | | | | | |
| | | involved in | | | | | | | |
| | | extracellular | | | | | | | |
| | | matrix | | | | | | | |
| | | organization | | | | | | | |
| upregulated | GO: 0035789 | metanephric | 139 | 15462 | 4 | 1 | 0.035480524 | 0.136159913 | ADIPOQ |
| | | mesenchymal | | | | | | | |
| | | cell migration | | | | | | | |
| upregulated | GO: 0061183 | regulation of | 139 | 15462 | 4 | 1 | 0.035480524 | 0.136159913 | WNT4 |
| | | dermatome | | | | | | | |
| | | development | | | | | | | |
| upregulated | GO: 2000467 | positive | 139 | 15462 | 4 | 1 | 0.035480524 | 0.136159913 | ADIPOQ |
| | | regulation of | | | | | | | |
| | | glycogen (starch) | | | | | | | |
| | | synthase activity | | | | | | | |
| upregulated | GO: 0030885 | regulation of | 139 | 15462 | 4 | 1 | 0.035480524 | 0.136159913 | CD2 |
| | | myeloid dendritic | | | | | | | |
| | | cell activation | | | | | | | |
| upregulated | GO: 0042816 | vitamin B6 | 139 | 15462 | 4 | 1 | 0.035480524 | 0.136159913 | AOX1 |
| | | metabolic | | | | | | | |
| | | process | | | | | | | |
| upregulated | GO: 0070050 | neuron cellular | 139 | 15462 | 4 | 1 | 0.035480524 | 0.136159913 | CHRNA1 |
| | | homeostasis | | | | | | | |
| upregulated | GO: 0060743 | epithelial cell | 139 | 15462 | 4 | 1 | 0.035480524 | 0.136159913 | FEM1B |
| | | maturation | | | | | | | |
| | | involved in | | | | | | | |
| | | prostate gland | | | | | | | |
| | | development | | | | | | | |
| upregulated | GO: 0060159 | regulation of | 139 | 15462 | 4 | 1 | 0.035480524 | 0.136159913 | LRRK2 |
| | | dopamine | | | | | | | |
| | | receptor | | | | | | | |
| | | signaling | | | | | | | |
| | | pathway | | | | | | | |
| upregulated | GO: 0042097 | interleukin-4 | 139 | 15462 | 4 | 1 | 0.035480524 | 0.136159913 | IRF4 |
| | | biosynthetic | | | | | | | |
| | | process | | | | | | | |
| upregulated | GO: 0061054 | dermatome | 139 | 15462 | 4 | 1 | 0.035480524 | 0.136159913 | WNT4 |
| | | development | | | | | | | |
| upregulated | GO: 0032966 | negative | 139 | 15462 | 4 | 1 | 0.035480524 | 0.136159913 | IL6 |
| | | regulation of | | | | | | | |
| | | collagen | | | | | | | |
| | | biosynthetic | | | | | | | |
| | | process | | | | | | | |
| upregulated | GO: 0035625 | epidermal growth | 139 | 15462 | 4 | 1 | 0.035480524 | 0.136159913 | ADRA2A |
| | | factor-activated | | | | | | | |
| | | receptor | | | | | | | |
| | | transactivation | | | | | | | |
| | | by G-protein | | | | | | | |
| | | coupled receptor | | | | | | | |
| | | signaling | | | | | | | |
| | | pathway | | | | | | | |
| upregulated | GO: 0014041 | regulation of | 139 | 15462 | 4 | 1 | 0.035480524 | 0.136159913 | LRRK2 |
| | | neuron | | | | | | | |
| | | maturation | | | | | | | |
| upregulated | GO: 0090403 | oxidative stress- | 139 | 15462 | 4 | 1 | 0.035480524 | 0.136159913 | PLA2R1 |
| | | induced | | | | | | | |
| | | premature | | | | | | | |
| | | senescence | | | | | | | |
| upregulated | GO: 0070278 | extracellular | 139 | 15462 | 4 | 1 | 0.035480524 | 0.136159913 | RGCC |
| | | matrix | | | | | | | |
| | | constituent | | | | | | | |
| | | secretion | | | | | | | |
| upregulated | GO: 1901725 | regulation of | 139 | 15462 | 4 | 1 | 0.035480524 | 0.136159913 | CAMK2D |
| | | histone | | | | | | | |
| | | deacetylase | | | | | | | |
| | | activity | | | | | | | |
| upregulated | GO: 0032345 | negative | 139 | 15462 | 4 | 1 | 0.035480524 | 0.136159913 | BMP2 |
| | | regulation of | | | | | | | |
| | | aldosterone | | | | | | | |
| | | metabolic | | | | | | | |
| | | process | | | | | | | |
| upregulated | GO: 2000589 | regulation of | 139 | 15462 | 4 | 1 | 0.035480524 | 0.136159913 | ADIPOQ |
| | | metanephric | | | | | | | |
| | | mesenchymal | | | | | | | |
| | | cell migration | | | | | | | |
| upregulated | GO: 0003166 | bundle of His | 139 | 15462 | 4 | 1 | 0.035480524 | 0.136159913 | TBX5 |
| | | development | | | | | | | |
| upregulated | GO: 0007525 | somatic muscle | 139 | 15462 | 4 | 1 | 0.035480524 | 0.136159913 | NKD1 |
| | | development | | | | | | | |
| upregulated | GO: 0060535 | trachea cartilage | 139 | 15462 | 4 | 1 | 0.035480524 | 0.136159913 | HOXA5 |
| | | morphogenesis | | | | | | | |
| upregulated | GO: 2000065 | negative | 139 | 15462 | 4 | 1 | 0.035480524 | 0.136159913 | BMP2 |
| | | regulation of | | | | | | | |
| | | cortisol | | | | | | | |
| | | biosynthetic | | | | | | | |
| | | process | | | | | | | |
| upregulated | GO: 0090238 | positive | 139 | 15462 | 4 | 1 | 0.035480524 | 0.136159913 | PLA2R1 |
| | | regulation of | | | | | | | |
| | | arachidonic acid | | | | | | | |
| | | secretion | | | | | | | |
| upregulated | GO: 0060546 | negative | 139 | 15462 | 4 | 1 | 0.035480524 | 0.136159913 | BIRC3 |
| | | regulation of | | | | | | | |
| | | necroptotic | | | | | | | |
| | | process | | | | | | | |
| upregulated | GO: 1902373 | negative | 139 | 15462 | 4 | 1 | 0.035480524 | 0.136159913 | DHX34 |
| | | regulation of | | | | | | | |
| | | mRNA catabolic | | | | | | | |
| | | process | | | | | | | |
| upregulated | GO: 0071447 | cellular response | 139 | 15462 | 4 | 1 | 0.035480524 | 0.136159913 | CD36 |
| | | to hydroperoxide | | | | | | | |
| upregulated | GO: 0070673 | response to | 139 | 15462 | 4 | 1 | 0.035480524 | 0.136159913 | IL18R1 |
| | | interleukin-18 | | | | | | | |
| upregulated | GO: 1903054 | negative | 139 | 15462 | 4 | 1 | 0.035480524 | 0.136159913 | DPP4 |
| | | regulation of | | | | | | | |
| | | extracellular | | | | | | | |
| | | matrix | | | | | | | |
| | | organization | | | | | | | |
| upregulated | GO: 1901148 | gene expression | 139 | 15462 | 4 | 1 | 0.035480524 | 0.136159913 | RGCC |
| | | involved in | | | | | | | |
| | | extracellular | | | | | | | |
| | | matrix | | | | | | | |
| | | organization | | | | | | | |
| upregulated | GO: 0010716 | negative | 139 | 15462 | 4 | 1 | 0.035480524 | 0.136159913 | DPP4 |
| | | regulation of | | | | | | | |
| | | extracellular | | | | | | | |
| | | matrix | | | | | | | |
| | | disassembly | | | | | | | |
| upregulated | GO: 0035788 | cell migration | 139 | 15462 | 4 | 1 | 0.035480524 | 0.136159913 | ADIPOQ |
| | | involved in | | | | | | | |
| | | metanephros | | | | | | | |
| | | development | | | | | | | |
| upregulated | GO: 0043654 | recognition of | 139 | 15462 | 4 | 1 | 0.035480524 | 0.136159913 | MEGF10 |
| | | apoptotic cell | | | | | | | |
| upregulated | GO: 0035701 | hematopoietic | 139 | 15462 | 4 | 1 | 0.035480524 | 0.136159913 | PTPRC |
| | | stem cell | | | | | | | |
| | | migration | | | | | | | |
| upregulated | GO: 0032348 | negative | 139 | 15462 | 4 | 1 | 0.035480524 | 0.136159913 | BMP2 |
| | | regulation of | | | | | | | |
| | | aldosterone | | | | | | | |
| | | biosynthetic | | | | | | | |
| | | process | | | | | | | |
| upregulated | GO: 0061205 | paramesonephric | 139 | 15462 | 4 | 1 | 0.035480524 | 0.136159913 | WNT4 |
| | | duct | | | | | | | |
| | | development | | | | | | | |
| upregulated | GO: 0042494 | detection of | 139 | 15462 | 4 | 1 | 0.035480524 | 0.136159913 | TLR1 |
| | | bacterial | | | | | | | |
| | | lipoprotein | | | | | | | |
| upregulated | GO: 0045402 | regulation of | 139 | 15462 | 4 | 1 | 0.035480524 | 0.136159913 | IRF4 |
| | | interleukin-4 | | | | | | | |
| | | biosynthetic | | | | | | | |
| | | process | | | | | | | |
| upregulated | GO: 1901313 | positive | 139 | 15462 | 4 | 1 | 0.035480524 | 0.136159913 | RGCC |
| | | regulation of | | | | | | | |
| | | gene expression | | | | | | | |
| | | involved in | | | | | | | |
| | | extracellular | | | | | | | |
| | | matrix | | | | | | | |
| | | organization | | | | | | | |
| upregulated | GO: 2000048 | negative | 139 | 15462 | 4 | 1 | 0.035480524 | 0.136159913 | RGCC |
| | | regulation of cell- | | | | | | | |
| | | cell adhesion | | | | | | | |
| | | mediated by | | | | | | | |
| | | cadherin | | | | | | | |
| upregulated | GO: 0032304 | negative | 139 | 15462 | 4 | 1 | 0.035480524 | 0.136159913 | PLA2R1 |
| | | regulation of | | | | | | | |
| | | icosanoid | | | | | | | |
| | | secretion | | | | | | | |
| upregulated | GO: 0016043 | cellular | 139 | 15462 | 4746 | 53 | 0.036428803 | 0.139171077 | DCLK1, SHOX2, IRF4, ADRA2A, |
| | | component | | | | | | | COL12A1, RAB30, BNIP3L, PDLIM5, |
| | | organization | | | | | | | MFAP5, GFRA1, NPTX1, DPP4, COL1A2, |
| | | | | | | | | | CAMK2D, ITGB3, RGCC, PRDM6, |
| | | | | | | | | | ERCC1, ASIC2, CNTN1, NR4A3, |
| | | | | | | | | | TSPAN5, TENM3, SEMA5A, CHRNA1, |
| | | | | | | | | | SOS1, ADAMTS5, EFEMP1, PLA2R1, |
| | | | | | | | | | ALOX5AP, CD36, MMP1, SATB2, |
| | | | | | | | | | ATP11A, CRISPLD2, LRRK2, ELN, |
| | | | | | | | | | STAP1, BIRC3, PRKG1, DLX5, MEGF10, |
| | | | | | | | | | CDH13, SDC2, ANGPTL4, BMP2, |
| | | | | | | | | | PTPRC, WNT4, IL6, TBX5, C1QTNF7, |
| | | | | | | | | | SKAP2, ADIPOQ |
| upregulated | GO: 0030111 | regulation of Wnt | 139 | 15462 | 203 | 5 | 0.036445854 | 0.139171077 | WNT4, MDFIC, NKD1, DLX5, BMP2 |
| | | signaling | | | | | | | |
| | | pathway | | | | | | | |
| upregulated | GO: 0032635 | interleukin-6 | 139 | 15462 | 81 | 3 | 0.036497047 | 0.139171077 | IL6, TLR1, CD36 |
| | | production | | | | | | | |
| upregulated | GO: 0045995 | regulation of | 139 | 15462 | 81 | 3 | 0.036497047 | 0.139171077 | WNT4, NKD1, ADIPOQ |
| | | embryonic | | | | | | | |
| | | development | | | | | | | |
| upregulated | GO: 0001678 | cellular glucose | 139 | 15462 | 81 | 3 | 0.036497047 | 0.139171077 | ADRA2A, NPTX1, FOXO1 |
| | | homeostasis | | | | | | | |
| upregulated | GO: 0097285 | cell-type specific | 139 | 15462 | 346 | 7 | 0.036851153 | 0.140343031 | RGCC, NR4A3, IL6, PDE1A, SEMA5A, |
| | | apoptotic | | | | | | | ANGPTL4, ADIPOQ |
| | | process | | | | | | | |
| upregulated | GO: 0001659 | temperature | 139 | 15462 | 34 | 2 | 0.037317295 | 0.141400508 | TNFRSF11A, FOXO1 |
| | | homeostasis | | | | | | | |
| upregulated | GO: 0051496 | positive | 139 | 15462 | 34 | 2 | 0.037317295 | 0.141400508 | RGCC, WNT4 |
| | | regulation of | | | | | | | |
| | | stress fiber | | | | | | | |
| | | assembly | | | | | | | |
| upregulated | GO: 0043502 | regulation of | 139 | 15462 | 34 | 2 | 0.037317295 | 0.141400508 | FBXO32, CAMK2D |
| | | muscle | | | | | | | |
| | | adaptation | | | | | | | |
| upregulated | GO: 0072210 | metanephric | 139 | 15462 | 34 | 2 | 0.037317295 | 0.141400508 | WNT4, ADIPOQ |
| | | nephron | | | | | | | |
| | | development | | | | | | | |
| upregulated | GO: 0033674 | positive | 139 | 15462 | 502 | 9 | 0.037478148 | 0.141830924 | LRRK2, BMP2, PTPRC, RGCC, ITGB3, |
| | | regulation of | | | | | | | TNFRSF11A, ADRA2A, MDFIC, ADIPOQ |
| | | kinase activity | | | | | | | |
| upregulated | GO: 0050773 | regulation of | 139 | 15462 | 82 | 3 | 0.037641257 | 0.142089827 | LRRK2, PDLIM5, SDC2 |
| | | dendrite | | | | | | | |
| | | development | | | | | | | |
| upregulated | GO: 0072006 | nephron | 139 | 15462 | 82 | 3 | 0.037641257 | 0.142089827 | WNT4, ADIPOQ, CALB1 |
| | | development | | | | | | | |
| upregulated | GO: 0043549 | regulation of | 139 | 15462 | 751 | 12 | 0.038142595 | 0.143801416 | LRRK2, CBLC, BMP2, FABP4, PTPRC, |
| | | kinase activity | | | | | | | RGCC, TNFRSF11A, ITGB3, ADRA2A, |
| | | | | | | | | | MDFIC, IL6, ADIPOQ |
| upregulated | GO: 0002224 | toll-like receptor | 139 | 15462 | 141 | 4 | 0.03842679 | 0.144511337 | BIRC3, TLR1, IRF4, CD36 |
| | | signaling | | | | | | | |
| | | pathway | | | | | | | |
| upregulated | GO: 0009059 | macromolecule | 139 | 15462 | 4446 | 50 | 0.038427207 | 0.144511337 | GPC6, FAM129A, SHOX2, IRF4, |
| | | biosynthetic | | | | | | | HS3ST3B1, FABP4, ADRA2A, TNNI2, |
| | | process | | | | | | | HOXA5, NFATC2, TFEC, NPAS2, GDF6, |
| | | | | | | | | | GALNT5, IL18R1, HIVEP3, CAMK2D, |
| | | | | | | | | | ITGB3, RGCC, PRDM6, BNC2, FOXO1, |
| | | | | | | | | | NR4A3, TLR1, PRELP, RFX2, RUNX1T1, |
| | | | | | | | | | EFEMP1, MDFIC, PPP1R3B, FOSL2, |
| | | | | | | | | | CD36, SATB2, ZFHX4, BIRC3, NR4A1, |
| | | | | | | | | | DLX5, SDC2, GCNT4, CDH13, BMP2, |
| | | | | | | | | | PTPRC, TNFRSF11A, GALNT14, WNT4, |
| | | | | | | | | | IL6, CHSY3, TBX5, NAMPT, ADIPOQ |
| upregulated | GO: 0044281 | small molecule | 139 | 15462 | 3115 | 37 | 0.038783441 | 0.145668468 | TAGAP, GPC6, HS3ST3B1, FABP4, |
| | | metabolic | | | | | | | ADRA2A, RAB30, NPAS2, GLRX, |
| | | process | | | | | | | RASGRF2, AOX1, ADH1B, CXCL9, |
| | | | | | | | | | BCHE, ARHGAP42, SLC22A3, CRLS1, |
| | | | | | | | | | PRELP, SOS1, ALOX5AP, CD36, DOCK8, |
| | | | | | | | | | LRRK2, PRKG1, GCNT4, PDE1A, SDC2, |
| | | | | | | | | | PLIN1, ANGPTL4, BMP2, GUCY1A3, |
| | | | | | | | | | WNT4, IL6, CHSY3, NAMPT, SLC7A5, |
| | | | | | | | | | TDO2, ADIPOQ |
| upregulated | GO: 0060828 | regulation of | 139 | 15462 | 142 | 4 | 0.039274808 | 0.146696497 | WNT4, NKD1, DLX5, BMP2 |
| | | canonical Wnt | | | | | | | |
| | | signaling | | | | | | | |
| | | pathway | | | | | | | |
| upregulated | GO: 0030890 | positive | 139 | 15462 | 35 | 2 | 0.039350443 | 0.146696497 | NFATC2, PTPRC |
| | | regulation of B | | | | | | | |
| | | cell proliferation | | | | | | | |
| upregulated | GO: 0034121 | regulation of toll- | 139 | 15462 | 35 | 2 | 0.039350443 | 0.146696497 | BIRC3, IRF4 |
| | | like receptor | | | | | | | |
| | | signaling | | | | | | | |
| | | pathway | | | | | | | |
| upregulated | GO: 0045912 | negative | 139 | 15462 | 35 | 2 | 0.039350443 | 0.146696497 | IL6, ADIPOQ |
| | | regulation of | | | | | | | |
| | | carbohydrate | | | | | | | |
| | | metabolic | | | | | | | |
| | | process | | | | | | | |
| upregulated | GO: 0051963 | regulation of | 139 | 15462 | 35 | 2 | 0.039350443 | 0.146696497 | ASIC2, PDLIM5 |
| | | synapse | | | | | | | |
| | | assembly | | | | | | | |
| upregulated | GO: 0050853 | B cell receptor | 139 | 15462 | 35 | 2 | 0.039350443 | 0.146696497 | NFATC2, PTPRC |
| | | signaling | | | | | | | |
| | | pathway | | | | | | | |
| upregulated | GO: 0017145 | stem cell division | 139 | 15462 | 143 | 4 | 0.040133494 | 0.149244876 | LRRK2, SHOX2, PTPRC, BMP2 |
| upregulated | GO: 0000302 | response to | 139 | 15462 | 143 | 4 | 0.040133494 | 0.149244876 | NR4A3, IL6, CD36, FOXO1 |
| | | reactive oxygen | | | | | | | |
| | | species | | | | | | | |
| upregulated | GO: 0048863 | stem cell | 139 | 15462 | 280 | 6 | 0.040889709 | 0.151868832 | RGCC, WNT4, TBX5, BCHE, BMP2, |
| | | differentiation | | | | | | | PTPRC |
| upregulated | GO: 0009896 | positive | 139 | 15462 | 144 | 4 | 0.041002851 | 0.151913031 | ADRA2A, LRRK2, NKD1, FOXO1 |
| | | regulation of | | | | | | | |
| | | catabolic | | | | | | | |
| | | process | | | | | | | |
| upregulated | GO: 0030336 | negative | 139 | 15462 | 144 | 4 | 0.041002851 | 0.151913031 | RGCC, WNT4, TBX5, ADIPOQ |
| | | regulation of cell | | | | | | | |
| | | migration | | | | | | | |
| upregulated | GO: 0046503 | glycerolipid | 139 | 15462 | 36 | 2 | 0.041424803 | 0.152161365 | PLIN1, FABP4 |
| | | catabolic | | | | | | | |
| | | process | | | | | | | |
| upregulated | GO: 0051101 | regulation of | 139 | 15462 | 36 | 2 | 0.041424803 | 0.152161365 | IRF4, RUNX1T1 |
| | | DNA binding | | | | | | | |
| upregulated | GO: 0060393 | regulation of | 139 | 15462 | 36 | 2 | 0.041424803 | 0.152161365 | GDF6, BMP2 |
| | | pathway- | | | | | | | |
| | | restricted SMAD | | | | | | | |
| | | protein | | | | | | | |
| | | phosphorylation | | | | | | | |
| upregulated | GO: 0032890 | regulation of | 139 | 15462 | 36 | 2 | 0.041424803 | 0.152161365 | TNFRSF11A, PLA2R1 |
| | | organic acid | | | | | | | |
| | | transport | | | | | | | |
| upregulated | GO: 0032760 | positive | 139 | 15462 | 36 | 2 | 0.041424803 | 0.152161365 | CD2, CD36 |
| | | regulation of | | | | | | | |
| | | tumor necrosis | | | | | | | |
| | | factor production | | | | | | | |
| upregulated | GO: 0045058 | T cell selection | 139 | 15462 | 36 | 2 | 0.041424803 | 0.152161365 | IL6, IRF4 |
| upregulated | GO: 0014909 | smooth muscle | 139 | 15462 | 36 | 2 | 0.041424803 | 0.152161365 | ITGB3, ADIPOQ |
| | | cell migration | | | | | | | |
| upregulated | GO: 0042098 | T cell | 139 | 15462 | 145 | 4 | 0.04188288 | 0.152362678 | WNT4, IL6, PTPRC, DOCK8 |
| | | proliferation | | | | | | | |
| upregulated | GO: 0048762 | mesenchymal | 139 | 15462 | 145 | 4 | 0.04188288 | 0.152362678 | RGCC, WNT4, TBX5, BMP2 |
| | | cell | | | | | | | |
| | | differentiation | | | | | | | |
| upregulated | GO: 0006816 | calcium ion | 139 | 15462 | 282 | 6 | 0.042086442 | 0.152362678 | ADRA2A, SLC24A3, CXCL9, CCR1, |
| | | transport | | | | | | | PTPRC, CAMK2D |
| upregulated | GO: 0044093 | positive | 139 | 15462 | 1573 | 21 | 0.042104745 | 0.152362678 | TAGAP, IRF4, SOS1, ADRA2A, MDFIC, |
| | | regulation of | | | | | | | ALOX5AP, DOCK8, LRRK2, BIRC3, |
| | | molecular | | | | | | | PDE1A, IL18R1, RASGRF2, PTPRC, |
| | | function | | | | | | | BMP2, TNFRSF11A, RGCC, ITGB3, |
| | | | | | | | | | WNT4, IL6, ARHGAP42, ADIPOQ |
| upregulated | GO: 0032940 | secretion by cell | 139 | 15462 | 763 | 12 | 0.04220459 | 0.152362678 | LRRK2, TLR1, CCR1, RGCC, CD2, |
| | | | | | | | | | ITGB3, ADRA2A, LCP2, IL6, CBLN4, |
| | | | | | | | | | CD36, ADIPOQ |
| upregulated | GO: 0045580 | regulation of T | 139 | 15462 | 86 | 3 | 0.042399769 | 0.152362678 | CD2, IL6, IRF4 |
| | | cell | | | | | | | |
| | | differentiation | | | | | | | |
| upregulated | GO: 0031329 | regulation of | 139 | 15462 | 679 | 11 | 0.042501579 | 0.152362678 | TAGAP, LRRK2, PRKG1, RASGRF2, |
| | | cellular catabolic | | | | | | | SOS1, DHX34, ADRA2A, WNT4, |
| | | process | | | | | | | PPP1R3B, ARHGAP42, DOCK8 |
| upregulated | GO: 0070838 | divalent metal | 139 | 15462 | 283 | 6 | 0.042693036 | 0.152362678 | ADRA2A, SLC24A3, CXCL9, CCR1, |
| | | ion transport | | | | | | | PTPRC, CAMK2D |
| upregulated | GO: 0050708 | regulation of | 139 | 15462 | 146 | 4 | 0.042773581 | 0.152362678 | RGCC, CD2, IL6, TLR1 |
| | | protein secretion | | | | | | | |
| upregulated | GO: 0051241 | negative | 139 | 15462 | 358 | 7 | 0.042979081 | 0.152362678 | RGCC, ADRA2A, IL6, TBX5, CCR1, |
| | | regulation of | | | | | | | GUCY1A3, ADIPOQ |
| | | multicellular | | | | | | | |
| | | organismal | | | | | | | |
| | | process | | | | | | | |
| upregulated | GO: 0046651 | lymphocyte | 139 | 15462 | 213 | 5 | 0.043333634 | 0.152362678 | WNT4, IL6, NFATC2, PTPRC, DOCK8 |
| | | proliferation | | | | | | | |
| upregulated | GO: 0051246 | regulation of | 139 | 15462 | 1862 | 24 | 0.043348754 | 0.152362678 | FAM129A, CBLC, TLR1, IRF4, FABP4, |
| | | protein metabolic | | | | | | | ADRA2A, MDFIC, FEM1B, CD36, LRRK2, |
| | | process | | | | | | | NKD1, BIRC3, NR4A1, GDF6, CAMK2D, |
| | | | | | | | | | PTPRC, BMP2, TNFRSF11A, ITGB3, |
| | | | | | | | | | RGCC, IL6, TFPI2, FOXO1, ADIPOQ |
| upregulated | GO: 0032755 | positive | 139 | 15462 | 37 | 2 | 0.043539482 | 0.152362678 | IL6, CD36 |
| | | regulation of | | | | | | | |
| | | interleukin-6 | | | | | | | |
| | | production | | | | | | | |
| upregulated | GO: 0014812 | muscle cell | 139 | 15462 | 37 | 2 | 0.043539482 | 0.152362678 | ITGB3, ADIPOQ |
| | | migration | | | | | | | |
| upregulated | GO: 0050881 | musculoskeletal | 139 | 15462 | 37 | 2 | 0.043539482 | 0.152362678 | TNNI2, CHRNA1 |
| | | movement | | | | | | | |
| upregulated | GO: 0050879 | multicellular | 139 | 15462 | 37 | 2 | 0.043539482 | 0.152362678 | TNNI2, CHRNA1 |
| | | organismal | | | | | | | |
| | | movement | | | | | | | |
| upregulated | GO: 0090090 | negative | 139 | 15462 | 87 | 3 | 0.043634461 | 0.152362678 | WNT4, NKD1, BMP2 |
| | | regulation of | | | | | | | |
| | | canonical Wnt | | | | | | | |
| | | signaling | | | | | | | |
| | | pathway | | | | | | | |
| upregulated | GO: 0034599 | cellular response | 139 | 15462 | 147 | 4 | 0.043674953 | 0.152362678 | IL6, PLA2R1, CD36, FOXO1 |
| | | to oxidative | | | | | | | |
| | | stress | | | | | | | |
| upregulated | GO: 1901897 | regulation of | 139 | 15462 | 5 | 1 | 0.044153587 | 0.152362678 | CAMK2D |
| | | relaxation of | | | | | | | |
| | | cardiac muscle | | | | | | | |
| upregulated | GO: 0060745 | mammary gland | 139 | 15462 | 5 | 1 | 0.044153587 | 0.152362678 | WNT4 |
| | | branching | | | | | | | |
| | | involved in | | | | | | | |
| | | pregnancy | | | | | | | |
| upregulated | GO: 2000096 | positive | 139 | 15462 | 5 | 1 | 0.044153587 | 0.152362678 | NKD1 |
| | | regulation of Wnt | | | | | | | |
| | | signaling | | | | | | | |
| | | pathway, planar | | | | | | | |
| | | cell polarity | | | | | | | |
| | | pathway | | | | | | | |
| upregulated | GO: 0010739 | positive | 139 | 15462 | 5 | 1 | 0.044153587 | 0.152362678 | ADIPOQ |
| | | regulation of | | | | | | | |
| | | protein kinase A | | | | | | | |
| | | signaling | | | | | | | |
| upregulated | GO: 1902803 | regulation of | 139 | 15462 | 5 | 1 | 0.044153587 | 0.152362678 | LRRK2 |
| | | synaptic vesicle | | | | | | | |
| | | transport | | | | | | | |
| upregulated | GO: 0060385 | axonogenesis | 139 | 15462 | 5 | 1 | 0.044153587 | 0.152362678 | NPTX1 |
| | | involved in | | | | | | | |
| | | innervation | | | | | | | |
| upregulated | GO: 0001781 | neutrophil | 139 | 15462 | 5 | 1 | 0.044153587 | 0.152362678 | IL6 |
| | | apoptotic | | | | | | | |
| | | process | | | | | | | |
| upregulated | GO: 0060534 | trachea cartilage | 139 | 15462 | 5 | 1 | 0.044153587 | 0.152362678 | HOXA5 |
| | | development | | | | | | | |
| upregulated | GO: 0072162 | metanephric | 139 | 15462 | 5 | 1 | 0.044153587 | 0.152362678 | WNT4 |
| | | mesenchymal | | | | | | | |
| | | cell | | | | | | | |
| | | differentiation | | | | | | | |
| upregulated | GO: 0060100 | positive | 139 | 15462 | 5 | 1 | 0.044153587 | 0.152362678 | CD36 |
| | | regulation of | | | | | | | |
| | | phagocytosis, | | | | | | | |
| | | engulfment | | | | | | | |
| upregulated | GO: 0070424 | regulation of | 139 | 15462 | 5 | 1 | 0.044153587 | 0.152362678 | BIRC3 |
| | | nucleotide- | | | | | | | |
| | | binding | | | | | | | |
| | | oligomerization | | | | | | | |
| | | domain | | | | | | | |
| | | containing | | | | | | | |
| | | signaling | | | | | | | |
| | | pathway | | | | | | | |
| upregulated | GO: 0060126 | somatotropin | 139 | 15462 | 5 | 1 | 0.044153587 | 0.152362678 | WNT4 |
| | | secreting cell | | | | | | | |
| | | differentiation | | | | | | | |
| upregulated | GO: 0061312 | BMP signaling | 139 | 15462 | 5 | 1 | 0.044153587 | 0.152362678 | BMP2 |
| | | pathway involved | | | | | | | |
| | | in heart | | | | | | | |
| | | development | | | | | | | |
| upregulated | GO: 0072221 | metanephric | 139 | 15462 | 5 | 1 | 0.044153587 | 0.152362678 | CALB1 |
| | | distal convoluted | | | | | | | |
| | | tubule | | | | | | | |
| | | development | | | | | | | |
| upregulated | GO: 0001302 | replicative cell | 139 | 15462 | 5 | 1 | 0.044153587 | 0.152362678 | ERCC1 |
| | | aging | | | | | | | |
| upregulated | GO: 0031944 | negative | 139 | 15462 | 5 | 1 | 0.044153587 | 0.152362678 | BMP2 |
| | | regulation of | | | | | | | |
| | | glucocorticoid | | | | | | | |
| | | metabolic | | | | | | | |
| | | process | | | | | | | |
| upregulated | GO: 0060480 | lung goblet cell | 139 | 15462 | 5 | 1 | 0.044153587 | 0.152362678 | HOXA5 |
| | | differentiation | | | | | | | |
| upregulated | GO: 0090032 | negative | 139 | 15462 | 5 | 1 | 0.044153587 | 0.152362678 | BMP2 |
| | | regulation of | | | | | | | |
| | | steroid hormone | | | | | | | |
| | | biosynthetic | | | | | | | |
| | | process | | | | | | | |
| upregulated | GO: 0072025 | distal convoluted | 139 | 15462 | 5 | 1 | 0.044153587 | 0.152362678 | CALB1 |
| | | tubule | | | | | | | |
| | | development | | | | | | | |
| upregulated | GO: 0072537 | fibroblast | 139 | 15462 | 5 | 1 | 0.044153587 | 0.152362678 | RGCC |
| | | activation | | | | | | | |
| upregulated | GO: 0010713 | negative | 139 | 15462 | 5 | 1 | 0.044153587 | 0.152362678 | IL6 |
| | | regulation of | | | | | | | |
| | | collagen | | | | | | | |
| | | metabolic | | | | | | | |
| | | process | | | | | | | |
| upregulated | GO: 0035564 | regulation of | 139 | 15462 | 5 | 1 | 0.044153587 | 0.152362678 | LRRK2 |
| | | kidney size | | | | | | | |
| upregulated | GO: 0051971 | positive | 139 | 15462 | 5 | 1 | 0.044153587 | 0.152362678 | IL6 |
| | | regulation of | | | | | | | |
| | | transmission of | | | | | | | |
| | | nerve impulse | | | | | | | |
| upregulated | GO: 0045475 | locomotor | 139 | 15462 | 5 | 1 | 0.044153587 | 0.152362678 | NPAS2 |
| | | rhythm | | | | | | | |
| upregulated | GO: 0035787 | cell migration | 139 | 15462 | 5 | 1 | 0.044153587 | 0.152362678 | ADIPOQ |
| | | involved in | | | | | | | |
| | | kidney | | | | | | | |
| | | development | | | | | | | |
| upregulated | GO: 0051005 | negative | 139 | 15462 | 5 | 1 | 0.044153587 | 0.152362678 | ANGPTL4 |
| | | regulation of | | | | | | | |
| | | lipoprotein lipase | | | | | | | |
| | | activity | | | | | | | |
| upregulated | GO: 0072033 | renal vesicle | 139 | 15462 | 5 | 1 | 0.044153587 | 0.152362678 | WNT4 |
| | | formation | | | | | | | |
| upregulated | GO: 0071455 | cellular response | 139 | 15462 | 5 | 1 | 0.044153587 | 0.152362678 | FOXO1 |
| | | to hyperoxia | | | | | | | |
| upregulated | GO: 1900121 | negative | 139 | 15462 | 5 | 1 | 0.044153587 | 0.152362678 | ADIPOQ |
| | | regulation of | | | | | | | |
| | | receptor binding | | | | | | | |
| upregulated | GO: 0031947 | negative | 139 | 15462 | 5 | 1 | 0.044153587 | 0.152362678 | BMP2 |
| | | regulation of | | | | | | | |
| | | glucocorticoid | | | | | | | |
| | | biosynthetic | | | | | | | |
| | | process | | | | | | | |
| upregulated | GO: 0035624 | receptor | 139 | 15462 | 5 | 1 | 0.044153587 | 0.152362678 | ADRA2A |
| | | transactivation | | | | | | | |
| upregulated | GO: 0090237 | regulation of | 139 | 15462 | 5 | 1 | 0.044153587 | 0.152362678 | PLA2R1 |
| | | arachidonic acid | | | | | | | |
| | | secretion | | | | | | | |
| upregulated | GO: 0036295 | cellular response | 139 | 15462 | 5 | 1 | 0.044153587 | 0.152362678 | FOXO1 |
| | | to increased | | | | | | | |
| | | oxygen levels | | | | | | | |
| upregulated | GO: 0060481 | lobar bronchus | 139 | 15462 | 5 | 1 | 0.044153587 | 0.152362678 | HOXA5 |
| | | epithelium | | | | | | | |
| | | development | | | | | | | |
| upregulated | GO: 0051128 | regulation of | 139 | 15462 | 1395 | 19 | 0.044170453 | 0.152362678 | SHOX2, TENM3, SEMA5A, PDLIM5, |
| | | cellular | | | | | | | CD36, LRRK2, ELN, SDC2, CDH13, |
| | | component | | | | | | | DPP4, CAMK2D, BMP2, RGCC, WNT4, |
| | | organization | | | | | | | ERCC1, TBX5, ASIC2, CNTN1, ADIPOQ |
| upregulated | GO: 0080090 | regulation of | 139 | 15462 | 5224 | 57 | 0.044364708 | 0.152857046 | TAGAP, FAM129A, CBLC, SHOX2, IRF4, |
| | | primary | | | | | | | FABP4, ADRA2A, TNNI2, HOXA5, |
| | | metabolic | | | | | | | NFATC2, TFEC, NPAS2, NKD1, GDF6, |
| | | process | | | | | | | RASGRF2, IL18R1, HIVEP3, CAMK2D, |
| | | | | | | | | | DHX34, ITGB3, RGCC, PRDM6, BNC2, |
| | | | | | | | | | ERCC1, TFPI2, CXCL9, FOXO1, |
| | | | | | | | | | ARHGAP42, NR4A3, TLR1, SOS1, |
| | | | | | | | | | RUNX1T1, RFX2, EFEMP1, MDFIC, |
| | | | | | | | | | FEM1B, PPP1R3B, FOSL2, CD36, |
| | | | | | | | | | SATB2, DOCK8, LRRK2, ZFHX4, BIRC3, |
| | | | | | | | | | NR4A1, PRKG1, DLX5, CDH13, BMP2, |
| | | | | | | | | | GUCY1A3, PTPRC, TNFRSF11A, WNT4, |
| | | | | | | | | | IL6, TBX5, NAMPT, ADIPOQ |
| upregulated | GO: 0051338 | regulation of | 139 | 15462 | 856 | 13 | 0.044527552 | 0.153242183 | LRRK2, CBLC, BMP2, FABP4, PTPRC, |
| | | transferase | | | | | | | RGCC, TNFRSF11A, ITGB3, ADRA2A, |
| | | activity | | | | | | | IL6, MDFIC, FEM1B, ADIPOQ |
| upregulated | GO: 2000146 | negative | 139 | 15462 | 148 | 4 | 0.044586992 | 0.153270977 | RGCC, WNT4, TBX5, ADIPOQ |
| | | regulation of cell | | | | | | | |
| | | motility | | | | | | | |
| upregulated | GO: 0032943 | mononuclear cell | 139 | 15462 | 215 | 5 | 0.044799042 | 0.153773934 | WNT4, IL6, NFATC2, PTPRC, DOCK8 |
| | | proliferation | | | | | | | |
| upregulated | GO: 0051238 | sequestering of | 139 | 15462 | 88 | 3 | 0.044887026 | 0.153773934 | CXCL9, PTPRC, CAMK2D |
| | | metal ion | | | | | | | |
| upregulated | GO: 0010594 | regulation of | 139 | 15462 | 88 | 3 | 0.044887026 | 0.153773934 | RGCC, ITGB3, SEMA5A |
| | | endothelial cell | | | | | | | |
| | | migration | | | | | | | |
| upregulated | GO: 0050865 | regulation of cell | 139 | 15462 | 362 | 7 | 0.045160341 | 0.154533848 | CD2, SLAMF7, IL6, NFATC2, IRF4, DPP4, |
| | | activation | | | | | | | PTPRC |
| upregulated | GO: 0002064 | epithelial cell | 139 | 15462 | 149 | 4 | 0.045509695 | 0.155101203 | WNT4, HOXA5, FEM1B, ADIPOQ |
| | | development | | | | | | | |
| upregulated | GO: 0051480 | cytosolic calcium | 139 | 15462 | 216 | 5 | 0.04554279 | 0.155101203 | CXCL9, PTPRC, CCR1, CALB1, CAMK2D |
| | | ion homeostasis | | | | | | | |
| upregulated | GO: 0035567 | non-canonical | 139 | 15462 | 38 | 2 | 0.045693598 | 0.155101203 | WNT4, NKD1 |
| | | Wnt signaling | | | | | | | |
| | | pathway | | | | | | | |
| upregulated | GO: 0060323 | head | 139 | 15462 | 38 | 2 | 0.045693598 | 0.155101203 | CRISPLD2, DLX5 |
| | | morphogenesis | | | | | | | |
| upregulated | GO: 1902930 | regulation of | 139 | 15462 | 38 | 2 | 0.045693598 | 0.155101203 | WNT4, BMP2 |
| | | alcohol | | | | | | | |
| | | biosynthetic | | | | | | | |
| | | process | | | | | | | |
| upregulated | GO: 0050982 | detection of | 139 | 15462 | 38 | 2 | 0.045693598 | 0.155101203 | ASIC2, PIEZO2 |
| | | mechanical | | | | | | | |
| | | stimulus | | | | | | | |
| upregulated | GO: 0032233 | positive | 139 | 15462 | 38 | 2 | 0.045693598 | 0.155101203 | RGCC, WNT4 |
| | | regulation of | | | | | | | |
| | | actin filament | | | | | | | |
| | | bundle assembly | | | | | | | |
| upregulated | GO: 0043269 | regulation of ion | 139 | 15462 | 441 | 8 | 0.045739608 | 0.155101203 | CCR1, CAMK2D, TNFRSF11A, ADRA2A, |
| | | transport | | | | | | | PLA2R1, ASIC2, CXCL9, CNTN1 |
| upregulated | GO: 0072511 | divalent | 139 | 15462 | 288 | 6 | 0.045808699 | 0.155160165 | ADRA2A, SLC24A3, CXCL9, CCR1, |
| | | inorganic cation | | | | | | | PTPRC, CAMK2D |
| | | transport | | | | | | | |
| upregulated | GO: 0061572 | actin filament | 139 | 15462 | 89 | 3 | 0.046157383 | 0.155813618 | RGCC, WNT4, ELN |
| | | bundle | | | | | | | |
| | | organization | | | | | | | |
| upregulated | GO: 0051017 | actin filament | 139 | 15462 | 89 | 3 | 0.046157383 | 0.155813618 | RGCC, WNT4, ELN |
| | | bundle assembly | | | | | | | |
| upregulated | GO: 0046631 | alpha-beta T cell | 139 | 15462 | 89 | 3 | 0.046157383 | 0.155813618 | IL6, IL18R1, IRF4 |
| | | activation | | | | | | | |
| upregulated | GO: 0002366 | leukocyte | 139 | 15462 | 150 | 4 | 0.046443055 | 0.156250682 | IL6, ERCC1, IL18R1, IRF4 |
| | | activation | | | | | | | |
| | | involved in | | | | | | | |
| | | immune | | | | | | | |
| | | response | | | | | | | |
| upregulated | GO: 0008217 | regulation of | 139 | 15462 | 150 | 4 | 0.046443055 | 0.156250682 | ASIC2, GUCY1A3, COL1A2, ADIPOQ |
| | | blood pressure | | | | | | | |
| upregulated | GO: 0002263 | cell activation | 139 | 15462 | 150 | 4 | 0.046443055 | 0.156250682 | IL6, ERCC1, IL18R1, IRF4 |
| | | involved in | | | | | | | |
| | | immune | | | | | | | |
| | | response | | | | | | | |
| upregulated | GO: 0048704 | embryonic | 139 | 15462 | 90 | 3 | 0.047445446 | 0.159444328 | HOXA5, SHOX2, SATB2 |
| | | skeletal system | | | | | | | |
| | | morphogenesis | | | | | | | |
| upregulated | GO: 0046637 | regulation of | 139 | 15462 | 39 | 2 | 0.047886283 | 0.160029773 | IL6, IRF4 |
| | | alpha-beta T cell | | | | | | | |
| | | differentiation | | | | | | | |
| upregulated | GO: 0030199 | collagen fibril | 139 | 15462 | 39 | 2 | 0.047886283 | 0.160029773 | COL12A1, COL1A2 |
| | | organization | | | | | | | |
| upregulated | GO: 0032330 | regulation of | 139 | 15462 | 39 | 2 | 0.047886283 | 0.160029773 | EFEMP1, SHOX2 |
| | | chondrocyte | | | | | | | |
| | | differentiation | | | | | | | |
| upregulated | GO: 0035136 | forelimb | 139 | 15462 | 39 | 2 | 0.047886283 | 0.160029773 | TBX5, SHOX2 |
| | | morphogenesis | | | | | | | |
| upregulated | GO: 0060996 | dendritic spine | 139 | 15462 | 39 | 2 | 0.047886283 | 0.160029773 | LRRK2, PDLIM5 |
| | | development | | | | | | | |
| upregulated | GO: 0050808 | synapse | 139 | 15462 | 152 | 4 | 0.048341721 | 0.16065726 | LRRK2, ASIC2, PDLIM5, CHRNA1 |
| | | organization | | | | | | | |
| upregulated | GO: 0002460 | adaptive immune | 139 | 15462 | 152 | 4 | 0.048341721 | 0.16065726 | IL6, ERCC1, IL18R1, IRF4 |
| | | response based | | | | | | | |
| | | on somatic | | | | | | | |
| | | recombination of | | | | | | | |
| | | immune | | | | | | | |
| | | receptors built | | | | | | | |
| | | from | | | | | | | |
| | | immunoglobulin | | | | | | | |
| | | superfamily | | | | | | | |
| | | domains | | | | | | | |
| upregulated | GO: 0042593 | glucose | 139 | 15462 | 152 | 4 | 0.048341721 | 0.16065726 | ADRA2A, NPTX1, ADIPOQ, FOXO1 |
| | | homeostasis | | | | | | | |
| upregulated | GO: 0048754 | branching | 139 | 15462 | 152 | 4 | 0.048341721 | 0.16065726 | WNT4, HOXA5, SEMA5A, BMP2 |
| | | morphogenesis | | | | | | | |
| | | of an epithelial | | | | | | | |
| | | tube | | | | | | | |
| upregulated | GO: 0033500 | carbohydrate | 139 | 15462 | 152 | 4 | 0.048341721 | 0.16065726 | ADRA2A, NPTX1, ADIPOQ, FOXO1 |
| | | homeostasis | | | | | | | |
| upregulated | GO: 0015850 | organic hydroxy | 139 | 15462 | 153 | 4 | 0.04930701 | 0.162432521 | ADRA2A, CD36, ADIPOQ, SLC22A3 |
| | | compound | | | | | | | |
| | | transport | | | | | | | |
| upregulated | GO: 0051271 | negative | 139 | 15462 | 153 | 4 | 0.04930701 | 0.162432521 | RGCC, WNT4, TBX5, ADIPOQ |
| | | regulation of | | | | | | | |
| | | cellular | | | | | | | |
| | | component | | | | | | | |
| | | movement | | | | | | | |
| upregulated | GO: 0002696 | positive | 139 | 15462 | 221 | 5 | 0.049372245 | 0.162432521 | CD2, IL6, NFATC2, DPP4, PTPRC |
| | | regulation of | | | | | | | |
| | | leukocyte | | | | | | | |
| | | activation | | | | | | | |
| downregulated | GO: 0008016 | regulation of | 30 | 15462 | 130 | 3 | 0.001997243 | 0.322191584 | CHRM2, MYH6, GNAO1 |
| | | heart contraction | | | | | | | |
| downregulated | GO: 0060047 | heart contraction | 30 | 15462 | 153 | 3 | 0.003170858 | 0.322191584 | CHRM2, MYH6, GNAO1 |
| downregulated | GO: 0003015 | heart process | 30 | 15462 | 154 | 3 | 0.003229644 | 0.322191584 | CHRM2, MYH6, GNAO1 |
| downregulated | GO: 0072560 | type B | 30 | 15462 | 2 | 1 | 0.003876842 | 0.322191584 | RFX3 |
| | | pancreatic cell | | | | | | | |
| | | maturation | | | | | | | |
| downregulated | GO: 0001895 | retina | 30 | 15462 | 55 | 2 | 0.005070011 | 0.322191584 | GPR98, AZGP1 |
| | | homeostasis | | | | | | | |
| downregulated | GO: 0002071 | glandular | 30 | 15462 | 3 | 1 | 0.005809811 | 0.322191584 | RFX3 |
| | | epithelial cell | | | | | | | |
| | | maturation | | | | | | | |
| downregulated | GO: 0007207 | phospholipase | 30 | 15462 | 3 | 1 | 0.005809811 | 0.322191584 | CHRM2 |
| | | C-activating G- | | | | | | | |
| | | protein coupled | | | | | | | |
| | | acetylcholine | | | | | | | |
| | | receptor | | | | | | | |
| | | signaling | | | | | | | |
| | | pathway | | | | | | | |
| downregulated | GO: 2000078 | positive | 30 | 15462 | 3 | 1 | 0.005809811 | 0.322191584 | RFX3 |
| | | regulation of type | | | | | | | |
| | | B pancreatic cell | | | | | | | |
| | | development | | | | | | | |
| downregulated | GO: 0060359 | response to | 30 | 15462 | 63 | 2 | 0.006604074 | 0.322191584 | GABRG2, GNAO1 |
| | | ammonium ion | | | | | | | |
| downregulated | GO: 0036101 | leukotriene B4 | 30 | 15462 | 4 | 1 | 0.007739153 | 0.322191584 | CYP4A11 |
| | | catabolic | | | | | | | |
| | | process | | | | | | | |
| downregulated | GO: 1901523 | icosanoid | 30 | 15462 | 4 | 1 | 0.007739153 | 0.322191584 | CYP4A11 |
| | | catabolic | | | | | | | |
| | | process | | | | | | | |
| downregulated | GO: 0036100 | leukotriene | 30 | 15462 | 4 | 1 | 0.007739153 | 0.322191584 | CYP4A11 |
| | | catabolic | | | | | | | |
| | | process | | | | | | | |
| downregulated | GO: 0036102 | leukotriene B4 | 30 | 15462 | 4 | 1 | 0.007739153 | 0.322191584 | CYP4A11 |
| | | metabolic | | | | | | | |
| | | process | | | | | | | |
| downregulated | GO: 0035524 | proline | 30 | 15462 | 4 | 1 | 0.007739153 | 0.322191584 | SLC6A15 |
| | | transmembrane | | | | | | | |
| | | transport | | | | | | | |
| downregulated | GO: 1901569 | fatty acid | 30 | 15462 | 4 | 1 | 0.007739153 | 0.322191584 | CYP4A11 |
| | | derivative | | | | | | | |
| | | catabolic | | | | | | | |
| | | process | | | | | | | |
| downregulated | GO: 0051570 | regulation of | 30 | 15462 | 5 | 1 | 0.009664876 | 0.322191584 | MYB |
| | | histone H3-K9 | | | | | | | |
| | | methylation | | | | | | | |
| downregulated | GO: 0051574 | positive | 30 | 15462 | 5 | 1 | 0.009664876 | 0.322191584 | MYB |
| | | regulation of | | | | | | | |
| | | histone H3-K9 | | | | | | | |
| | | methylation | | | | | | | |
| downregulated | GO: 0002069 | columnar/cuboidal | 30 | 15462 | 6 | 1 | 0.011586986 | 0.322191584 | RFX3 |
| | | epithelial cell | | | | | | | |
| | | maturation | | | | | | | |
| downregulated | GO: 0055009 | atrial cardiac | 30 | 15462 | 6 | 1 | 0.011586986 | 0.322191584 | MYH6 |
| | | muscle tissue | | | | | | | |
| | | morphogenesis | | | | | | | |
| downregulated | GO: 0003228 | atrial cardiac | 30 | 15462 | 6 | 1 | 0.011586986 | 0.322191584 | MYH6 |
| | | muscle tissue | | | | | | | |
| | | development | | | | | | | |
| downregulated | GO: 0042758 | long-chain fatty | 30 | 15462 | 7 | 1 | 0.013505489 | 0.322191584 | CYP4A11 |
| | | acid catabolic | | | | | | | |
| | | process | | | | | | | |
| downregulated | GO: 0071420 | cellular response | 30 | 15462 | 7 | 1 | 0.013505489 | 0.322191584 | GABRG2 |
| | | to histamine | | | | | | | |
| downregulated | GO: 2000074 | regulation of type | 30 | 15462 | 7 | 1 | 0.013505489 | 0.322191584 | RFX3 |
| | | B pancreatic cell | | | | | | | |
| | | development | | | | | | | |
| downregulated | GO: 0045162 | clustering of | 30 | 15462 | 7 | 1 | 0.013505489 | 0.322191584 | GLDN |
| | | voltage-gated | | | | | | | |
| | | sodium channels | | | | | | | |
| downregulated | GO: 0060287 | epithelial cilium | 30 | 15462 | 7 | 1 | 0.013505489 | 0.322191584 | RFX3 |
| | | movement | | | | | | | |
| | | involved in | | | | | | | |
| | | determination of | | | | | | | |
| | | left/right | | | | | | | |
| | | asymmetry | | | | | | | |
| downregulated | GO: 0003008 | system process | 30 | 15462 | 1705 | 8 | 0.013653606 | 0.322191584 | OR52L1, CHRM2, CYP4A11, MYH6, |
| | | | | | | | | | GPR98, GNAO1, RBFOX1, AZGP1 |
| downregulated | GO: 0048739 | cardiac muscle | 30 | 15462 | 8 | 1 | 0.015420393 | 0.322191584 | MYH6 |
| | | fiber | | | | | | | |
| | | development | | | | | | | |
| downregulated | GO: 0032528 | microvillus | 30 | 15462 | 8 | 1 | 0.015420393 | 0.322191584 | GLDN |
| | | organization | | | | | | | |
| downregulated | GO: 0015824 | proline transport | 30 | 15462 | 8 | 1 | 0.015420393 | 0.322191584 | SLC6A15 |
| downregulated | GO: 0060285 | cilium-dependent | 30 | 15462 | 8 | 1 | 0.015420393 | 0.322191584 | RFX3 |
| | | cell motility | | | | | | | |
| downregulated | GO: 0007186 | G-protein | 30 | 15462 | 1087 | 6 | 0.016448265 | 0.322191584 | GABRG2, OR52L1, NPY6R, CHRM2, |
| | | coupled receptor | | | | | | | GNAO1, GPR98 |
| | | signaling | | | | | | | |
| | | pathway | | | | | | | |
| downregulated | GO: 0006816 | calcium ion | 30 | 15462 | 282 | 3 | 0.016942615 | 0.322191584 | MYB, GNAO1, CACHD1 |
| | | transport | | | | | | | |
| downregulated | GO: 0070838 | divalent metal | 30 | 15462 | 283 | 3 | 0.017101974 | 0.322191584 | MYB, GNAO1, CACHD1 |
| | | ion transport | | | | | | | |
| downregulated | GO: 0097267 | omega- | 30 | 15462 | 9 | 1 | 0.017331703 | 0.322191584 | CYP4A11 |
| | | hydroxylase | | | | | | | |
| | | P450 pathway | | | | | | | |
| downregulated | GO: 0003091 | renal water | 30 | 15462 | 9 | 1 | 0.017331703 | 0.322191584 | CYP4A11 |
| | | homeostasis | | | | | | | |
| downregulated | GO: 0006703 | estrogen | 30 | 15462 | 9 | 1 | 0.017331703 | 0.322191584 | CYP19A1 |
| | | biosynthetic | | | | | | | |
| | | process | | | | | | | |
| downregulated | GO: 0002699 | positive | 30 | 15462 | 106 | 2 | 0.017873443 | 0.322191584 | MYB, AZGP1 |
| | | regulation of | | | | | | | |
| | | immune effector | | | | | | | |
| | | process | | | | | | | |
| downregulated | GO: 0072511 | divalent | 30 | 15462 | 288 | 3 | 0.017911507 | 0.322191584 | MYB, GNAO1, CACHD1 |
| | | inorganic cation | | | | | | | |
| | | transport | | | | | | | |
| downregulated | GO: 0048469 | cell maturation | 30 | 15462 | 109 | 2 | 0.018836785 | 0.322191584 | GLDN, RFX3 |
| downregulated | GO: 0034776 | response to | 30 | 15462 | 10 | 1 | 0.019239427 | 0.322191584 | GABRG2 |
| | | histamine | | | | | | | |
| downregulated | GO: 0019373 | epoxygenase | 30 | 15462 | 10 | 1 | 0.019239427 | 0.322191584 | CYP4A11 |
| | | P450 pathway | | | | | | | |
| downregulated | GO: 0051571 | positive | 30 | 15462 | 10 | 1 | 0.019239427 | 0.322191584 | MYB |
| | | regulation of | | | | | | | |
| | | histone H3-K4 | | | | | | | |
| | | methylation | | | | | | | |
| downregulated | GO: 0060736 | prostate gland | 30 | 15462 | 10 | 1 | 0.019239427 | 0.322191584 | CYP19A1 |
| | | growth | | | | | | | |
| downregulated | GO: 0007188 | adenylate | 30 | 15462 | 118 | 2 | 0.021855358 | 0.322191584 | CHRM2, GNAO1 |
| | | cyclase- | | | | | | | |
| | | modulating G- | | | | | | | |
| | | protein coupled | | | | | | | |
| | | receptor | | | | | | | |
| | | signaling | | | | | | | |
| | | pathway | | | | | | | |
| downregulated | GO: 0007213 | G-protein | 30 | 15462 | 12 | 1 | 0.023044139 | 0.322191584 | CHRM2 |
| | | coupled | | | | | | | |
| | | acetylcholine | | | | | | | |
| | | receptor | | | | | | | |
| | | signaling | | | | | | | |
| | | pathway | | | | | | | |
| downregulated | GO: 0032305 | positive | 30 | 15462 | 12 | 1 | 0.023044139 | 0.322191584 | CYP4A11 |
| | | regulation of | | | | | | | |
| | | icosanoid | | | | | | | |
| | | secretion | | | | | | | |
| downregulated | GO: 0045624 | positive | 30 | 15462 | 12 | 1 | 0.023044139 | 0.322191584 | MYB |
| | | regulation of T- | | | | | | | |
| | | helper cell | | | | | | | |
| | | differentiation | | | | | | | |
| downregulated | GO: 0045161 | neuronal ion | 30 | 15462 | 12 | 1 | 0.023044139 | 0.322191584 | GLDN |
| | | channel | | | | | | | |
| | | clustering | | | | | | | |
| downregulated | GO: 0060122 | inner ear | 30 | 15462 | 12 | 1 | 0.023044139 | 0.322191584 | GPR98 |
| | | receptor | | | | | | | |
| | | stereocilium | | | | | | | |
| | | organization | | | | | | | |
| downregulated | GO: 0071242 | cellular response | 30 | 15462 | 13 | 1 | 0.02494114 | 0.322191584 | GABRG2 |
| | | to ammonium ion | | | | | | | |
| downregulated | GO: 0044057 | regulation of | 30 | 15462 | 333 | 3 | 0.026158901 | 0.322191584 | CHRM2, MYH6, GNAO1 |
| | | system process | | | | | | | |
| downregulated | GO: 0050910 | detection of | 30 | 15462 | 14 | 1 | 0.026834581 | 0.322191584 | GPR98 |
| | | mechanical | | | | | | | |
| | | stimulus involved | | | | | | | |
| | | in sensory | | | | | | | |
| | | perception of | | | | | | | |
| | | sound | | | | | | | |
| downregulated | GO: 2000193 | positive | 30 | 15462 | 14 | 1 | 0.026834581 | 0.322191584 | CYP4A11 |
| | | regulation of fatty | | | | | | | |
| | | acid transport | | | | | | | |
| downregulated | GO: 0007512 | adult heart | 30 | 15462 | 14 | 1 | 0.026834581 | 0.322191584 | MYH6 |
| | | development | | | | | | | |
| downregulated | GO: 0043949 | regulation of | 30 | 15462 | 133 | 2 | 0.027299117 | 0.322191584 | CHRM2, GNAO1 |
| | | cAMP-mediated | | | | | | | |
| | | signaling | | | | | | | |
| downregulated | GO: 0032303 | regulation of | 30 | 15462 | 15 | 1 | 0.028724467 | 0.322191584 | CYP4A11 |
| | | icosanoid | | | | | | | |
| | | secretion | | | | | | | |
| downregulated | GO: 0002070 | epithelial cell | 30 | 15462 | 15 | 1 | 0.028724467 | 0.322191584 | RFX3 |
| | | maturation | | | | | | | |
| downregulated | GO: 0043372 | positive | 30 | 15462 | 15 | 1 | 0.028724467 | 0.322191584 | MYB |
| | | regulation of | | | | | | | |
| | | CD4-positive, | | | | | | | |
| | | alpha-beta T cell | | | | | | | |
| | | differentiation | | | | | | | |
| downregulated | GO: 0071705 | nitrogen | 30 | 15462 | 608 | 4 | 0.028833028 | 0.322191584 | SLC6A15, RFX3, SYBU, RBFOX1 |
| | | compound | | | | | | | |
| | | transport | | | | | | | |
| downregulated | GO: 0003323 | type B | 30 | 15462 | 16 | 1 | 0.030610806 | 0.322191584 | RFX3 |
| | | pancreatic cell | | | | | | | |
| | | development | | | | | | | |
| downregulated | GO: 0051567 | histone H3-K9 | 30 | 15462 | 16 | 1 | 0.030610806 | 0.322191584 | MYB |
| | | methylation | | | | | | | |
| downregulated | GO: 0045761 | regulation of | 30 | 15462 | 147 | 2 | 0.032821306 | 0.322191584 | CHRM2, GNAO1 |
| | | adenylate | | | | | | | |
| | | cyclase activity | | | | | | | |
| downregulated | GO: 0006805 | xenobiotic | 30 | 15462 | 148 | 2 | 0.033231367 | 0.322191584 | CYP19A1, CYP4A11 |
| | | metabolic | | | | | | | |
| | | process | | | | | | | |
| downregulated | GO: 0071466 | cellular response | 30 | 15462 | 149 | 2 | 0.033643463 | 0.322191584 | CYP19A1, CYP4A11 |
| | | to xenobiotic | | | | | | | |
| | | stimulus | | | | | | | |
| downregulated | GO: 0007187 | G-protein | 30 | 15462 | 149 | 2 | 0.033643463 | 0.322191584 | CHRM2, GNAO1 |
| | | coupled receptor | | | | | | | |
| | | signaling | | | | | | | |
| | | pathway, | | | | | | | |
| | | coupled to cyclic | | | | | | | |
| | | nucleotide | | | | | | | |
| | | second | | | | | | | |
| | | messenger | | | | | | | |
| downregulated | GO: 0031062 | positive | 30 | 15462 | 18 | 1 | 0.034372863 | 0.322191584 | MYB |
| | | regulation of | | | | | | | |
| | | histone | | | | | | | |
| | | methylation | | | | | | | |
| downregulated | GO: 0006691 | leukotriene | 30 | 15462 | 18 | 1 | 0.034372863 | 0.322191584 | CYP4A11 |
| | | metabolic | | | | | | | |
| | | process | | | | | | | |
| downregulated | GO: 0036465 | synaptic vesicle | 30 | 15462 | 18 | 1 | 0.034372863 | 0.322191584 | SH3GL2 |
| | | recycling | | | | | | | |
| downregulated | GO: 0048488 | synaptic vesicle | 30 | 15462 | 18 | 1 | 0.034372863 | 0.322191584 | SH3GL2 |
| | | endocytosis | | | | | | | |
| downregulated | GO: 0009083 | branched-chain | 30 | 15462 | 18 | 1 | 0.034372863 | 0.322191584 | HIBCH |
| | | amino acid | | | | | | | |
| | | catabolic | | | | | | | |
| | | process | | | | | | | |
| downregulated | GO: 2000516 | positive | 30 | 15462 | 18 | 1 | 0.034372863 | 0.322191584 | MYB |
| | | regulation of | | | | | | | |
| | | CD4-positive, | | | | | | | |
| | | alpha-beta T cell | | | | | | | |
| | | activation | | | | | | | |
| downregulated | GO: 0051569 | regulation of | 30 | 15462 | 18 | 1 | 0.034372863 | 0.322191584 | MYB |
| | | histone H3-K4 | | | | | | | |
| | | methylation | | | | | | | |
| downregulated | GO: 0002068 | glandular | 30 | 15462 | 18 | 1 | 0.034372863 | 0.322191584 | RFX3 |
| | | epithelial cell | | | | | | | |
| | | development | | | | | | | |
| downregulated | GO: 0019933 | cAMP-mediated | 30 | 15462 | 151 | 2 | 0.034473722 | 0.322191584 | CHRM2, GNAO1 |
| | | signaling | | | | | | | |
| downregulated | GO: 0009410 | response to | 30 | 15462 | 152 | 2 | 0.034891869 | 0.322191584 | CYP19A1, CYP4A11 |
| | | xenobiotic | | | | | | | |
| | | stimulus | | | | | | | |
| downregulated | GO: 0008015 | blood circulation | 30 | 15462 | 374 | 3 | 0.035184232 | 0.322191584 | CHRM2, MYH6, GNAO1 |
| downregulated | GO: 0003013 | circulatory | 30 | 15462 | 376 | 3 | 0.035661162 | 0.322191584 | CHRM2, MYH6, GNAO1 |
| | | system process | | | | | | | |
| downregulated | GO: 0055008 | cardiac muscle | 30 | 15462 | 19 | 1 | 0.036248596 | 0.322191584 | MYH6 |
| | | tissue | | | | | | | |
| | | morphogenesis | | | | | | | |
| downregulated | GO: 0001916 | positive | 30 | 15462 | 19 | 1 | 0.036248596 | 0.322191584 | AZGP1 |
| | | regulation of T | | | | | | | |
| | | cell mediated | | | | | | | |
| | | cytotoxicity | | | | | | | |
| downregulated | GO: 0060420 | regulation of | 30 | 15462 | 19 | 1 | 0.036248596 | 0.322191584 | MYH6 |
| | | heart growth | | | | | | | |
| downregulated | GO: 0015804 | neutral amino | 30 | 15462 | 19 | 1 | 0.036248596 | 0.322191584 | SLC6A15 |
| | | acid transport | | | | | | | |
| downregulated | GO: 0003351 | epithelial cilium | 30 | 15462 | 19 | 1 | 0.036248596 | 0.322191584 | RFX3 |
| | | movement | | | | | | | |
| downregulated | GO: 0006811 | ion transport | 30 | 15462 | 1311 | 6 | 0.037364547 | 0.322191584 | GABRG2, SLC6A15, CYP4A11, MYB, |
| | | | | | | | | | GNAO1, CACHD1 |
| downregulated | GO: 0098656 | anion | 30 | 15462 | 158 | 2 | 0.037442429 | 0.322191584 | GABRG2, SLC6A15 |
| | | transmembrane | | | | | | | |
| | | transport | | | | | | | |
| downregulated | GO: 0008210 | estrogen | 30 | 15462 | 20 | 1 | 0.038120806 | 0.322191584 | CYP19A1 |
| | | metabolic | | | | | | | |
| | | process | | | | | | | |
| downregulated | GO: 0003309 | type B | 30 | 15462 | 20 | 1 | 0.038120806 | 0.322191584 | RFX3 |
| | | pancreatic cell | | | | | | | |
| | | differentiation | | | | | | | |
| downregulated | GO: 0001914 | regulation of T | 30 | 15462 | 20 | 1 | 0.038120806 | 0.322191584 | AZGP1 |
| | | cell mediated | | | | | | | |
| | | cytotoxicity | | | | | | | |
| downregulated | GO: 0030001 | metal ion | 30 | 15462 | 672 | 4 | 0.039537437 | 0.322191584 | SLC6A15, MYB, GNAO1, CACHD1 |
| | | transport | | | | | | | |
| downregulated | GO: 0050796 | regulation of | 30 | 15462 | 163 | 2 | 0.039621523 | 0.322191584 | RFX3, SYBU |
| | | insulin secretion | | | | | | | |
| downregulated | GO: 0009081 | branched-chain | 30 | 15462 | 21 | 1 | 0.0399895 | 0.322191584 | HIBCH |
| | | amino acid | | | | | | | |
| | | metabolic | | | | | | | |
| | | process | | | | | | | |
| downregulated | GO: 0007214 | gamma- | 30 | 15462 | 21 | 1 | 0.0399895 | 0.322191584 | GABRG2 |
| | | aminobutyric | | | | | | | |
| | | acid signaling | | | | | | | |
| | | pathway | | | | | | | |
| downregulated | GO: 0002026 | regulation of the | 30 | 15462 | 21 | 1 | 0.0399895 | 0.322191584 | MYH6 |
| | | force of heart | | | | | | | |
| | | contraction | | | | | | | |
| downregulated | GO: 0031279 | regulation of | 30 | 15462 | 166 | 2 | 0.040951901 | 0.322191584 | CHRM2, GNAO1 |
| | | cyclase activity | | | | | | | |
| downregulated | GO: 0030817 | regulation of | 30 | 15462 | 168 | 2 | 0.041848227 | 0.322191584 | CHRM2, GNAO1 |
| | | cAMP | | | | | | | |
| | | biosynthetic | | | | | | | |
| | | process | | | | | | | |
| downregulated | GO: 2000191 | regulation of fatty | 30 | 15462 | 22 | 1 | 0.041854685 | 0.322191584 | CYP4A11 |
| | | acid transport | | | | | | | |
| downregulated | GO: 0035883 | enteroendocrine | 30 | 15462 | 22 | 1 | 0.041854685 | 0.322191584 | RFX3 |
| | | cell | | | | | | | |
| | | differentiation | | | | | | | |
| downregulated | GO: 0032892 | positive | 30 | 15462 | 22 | 1 | 0.041854685 | 0.322191584 | CYP4A11 |
| | | regulation of | | | | | | | |
| | | organic acid | | | | | | | |
| | | transport | | | | | | | |
| downregulated | GO: 0045622 | regulation of T- | 30 | 15462 | 22 | 1 | 0.041854685 | 0.322191584 | MYB |
| | | helper cell | | | | | | | |
| | | differentiation | | | | | | | |
| downregulated | GO: 0051926 | negative | 30 | 15462 | 22 | 1 | 0.041854685 | 0.322191584 | GNAO1 |
| | | regulation of | | | | | | | |
| | | calcium ion | | | | | | | |
| | | transport | | | | | | | |
| downregulated | GO: 0007212 | dopamine | 30 | 15462 | 22 | 1 | 0.041854685 | 0.322191584 | GNAO1 |
| | | receptor | | | | | | | |
| | | signaling | | | | | | | |
| | | pathway | | | | | | | |
| downregulated | GO: 0001539 | cilium or | 30 | 15462 | 22 | 1 | 0.041854685 | 0.322191584 | RFX3 |
| | | flagellum- | | | | | | | |
| | | dependent cell | | | | | | | |
| | | motility | | | | | | | |
| downregulated | GO: 0051339 | regulation of | 30 | 15462 | 169 | 2 | 0.042299188 | 0.322191584 | CHRM2, GNAO1 |
| | | lyase activity | | | | | | | |
| downregulated | GO: 0006171 | cAMP | 30 | 15462 | 170 | 2 | 0.042752003 | 0.322191584 | CHRM2, GNAO1 |
| | | biosynthetic | | | | | | | |
| | | process | | | | | | | |
| downregulated | GO: 0010927 | cellular | 30 | 15462 | 171 | 2 | 0.043206663 | 0.322191584 | RFX3, MYH6 |
| | | component | | | | | | | |
| | | assembly | | | | | | | |
| | | involved in | | | | | | | |
| | | morphogenesis | | | | | | | |
| downregulated | GO: 0001894 | tissue | 30 | 15462 | 172 | 2 | 0.043663159 | 0.322191584 | GPR98, AZGP1 |
| | | homeostasis | | | | | | | |
| downregulated | GO: 0021700 | developmental | 30 | 15462 | 174 | 2 | 0.044581631 | 0.322191584 | GLDN, RFX3 |
| | | maturation | | | | | | | |
| downregulated | GO: 0046395 | carboxylic acid | 30 | 15462 | 175 | 2 | 0.045043589 | 0.322191584 | CYP4A11, HIBCH |
| | | catabolic | | | | | | | |
| | | process | | | | | | | |
| downregulated | GO: 0016054 | organic acid | 30 | 15462 | 175 | 2 | 0.045043589 | 0.322191584 | CYP4A11, HIBCH |
| | | catabolic | | | | | | | |
| | | process | | | | | | | |
| downregulated | GO: 0030030 | cell projection | 30 | 15462 | 1026 | 5 | 0.045494482 | 0.322191584 | GLDN, RFX3, GNAO1, GPR98, SH3GL2 |
| | | organization | | | | | | | |
| downregulated | GO: 0060119 | inner ear | 30 | 15462 | 24 | 1 | 0.045574551 | 0.322191584 | GPR98 |
| | | receptor cell | | | | | | | |
| | | development | | | | | | | |
| downregulated | GO: 0050974 | detection of | 30 | 15462 | 24 | 1 | 0.045574551 | 0.322191584 | GPR98 |
| | | mechanical | | | | | | | |
| | | stimulus involved | | | | | | | |
| | | in sensory | | | | | | | |
| | | perception | | | | | | | |
| downregulated | GO: 0010817 | regulation of | 30 | 15462 | 416 | 3 | 0.04590643 | 0.322191584 | CYP19A1, RFX3, SYBU |
| | | hormone levels | | | | | | | |
| downregulated | GO: 0090276 | regulation of | 30 | 15462 | 179 | 2 | 0.046909379 | 0.322191584 | RFX3, SYBU |
| | | peptide hormone | | | | | | | |
| | | secretion | | | | | | | |
| downregulated | GO: 0043278 | response to | 30 | 15462 | 25 | 1 | 0.047429245 | 0.322191584 | GNAO1 |
| | | morphine | | | | | | | |
| downregulated | GO: 0001913 | T cell mediated | 30 | 15462 | 25 | 1 | 0.047429245 | 0.322191584 | AZGP1 |
| | | cytotoxicity | | | | | | | |
| downregulated | GO: 0034113 | heterotypic cell- | 30 | 15462 | 25 | 1 | 0.047429245 | 0.322191584 | GLDN |
| | | cell adhesion | | | | | | | |
| downregulated | GO: 0060415 | muscle tissue | 30 | 15462 | 25 | 1 | 0.047429245 | 0.322191584 | MYH6 |
| | | morphogenesis | | | | | | | |
| downregulated | GO: 0030814 | regulation of | 30 | 15462 | 181 | 2 | 0.047852934 | 0.322191584 | CHRM2, GNAO1 |
| | | cAMP metabolic | | | | | | | |
| | | process | | | | | | | |
| downregulated | GO: 0006810 | transport | 30 | 15462 | 3838 | 12 | 0.048055387 | 0.322191584 | SLC6A15, FNBP1, SH3GL2, GABRG2, |
| | | | | | | | | | RFX3, CYP4A11, SYBU, MYB, RBFOX1, |
| | | | | | | | | | GNAO1, CACHD1, AZGP1 |
| downregulated | GO: 0048002 | antigen | 30 | 15462 | 183 | 2 | 0.048803509 | 0.322191584 | SH3GL2, AZGP1 |
| | | processing and | | | | | | | |
| | | presentation of | | | | | | | |
| | | peptide antigen | | | | | | | |
| downregulated | GO: 0050906 | detection of | 30 | 15462 | 428 | 3 | 0.049239168 | 0.322191584 | OR52L1, GPR98, AZGP1 |
| | | stimulus involved | | | | | | | |
| | | in sensory | | | | | | | |
| | | perception | | | | | | | |
| downregulated | GO: 0002090 | regulation of | 30 | 15462 | 26 | 1 | 0.049280454 | 0.322191584 | SH3GL2 |
| | | receptor | | | | | | | |
| | | internalization | | | | | | | |
| downregulated | GO: 0043370 | regulation of | 30 | 15462 | 26 | 1 | 0.049280454 | 0.322191584 | MYB |
| | | CD4-positive, | | | | | | | |
| | | alpha-beta T cell | | | | | | | |
| | | differentiation | | | | | | | |
| downregulated | GO: 0003209 | cardiac atrium | 30 | 15462 | 26 | 1 | 0.049280454 | 0.322191584 | MYH6 |
| | | morphogenesis | | | | | | | |
| downregulated | GO: 0014072 | response to | 30 | 15462 | 26 | 1 | 0.049280454 | 0.322191584 | GNAO1 |
| | | isoquinoline | | | | | | | |
| | | alkaloid | | | | | | | |
| downregulated | GO: 0045494 | photoreceptor | 30 | 15462 | 26 | 1 | 0.049280454 | 0.322191584 | GPR98 |
| | | cell maintenance | | | | | | | |
| downregulated | GO: 0002791 | regulation of | 30 | 15462 | 184 | 2 | 0.049281409 | 0.322191584 | RFX3, SYBU |
| | | peptide secretion | | | | | | | |

TABLE 11
AngioImmune Probeset Information

| | | | No. | | | | | | | |
|---|---|---|---|---|---|---|---|---|---|---|
| | | | pro- | | | | | | | |
| Probe | | | bes | | Gene | Entrez | | Chro- | | SEQ |
| Set | | Orien- | alig- | Ensembl | Sym- | Gene | | mo- | | ID |
| ID | Type | tation | ned | Gene | bol | ID | Description | some | Strand | NO |
| OC3P. | Expression | Sense | 11 | ENSG00000- | ADAM8 | 101 | ADAM metallopeptidase | Chr | Reverse | 303 |
| 7013. | probeset | (Fully | | 151651 | | | domain 8 [Source: HGNC | 10 | Strand | |
| C1_s_at | | Exonic) | | | | | Symbol; Acc: 215] | | | |
| OC3P. | Expression | Sense | 11 | ENSG00000- | ALOX5 | 240 | arachidonate 5- | Chr | Forward | 304 |
| 10546. | probeset | (Fully | | 012779 | | | lipoxygenase | 10 | Strand | |
| C1_s_at | | Exonic) | | | | | [Source: HGNC | | | |
| | | | | | | | Symbol; Acc: 435] | | | |
| OC3SNGn. | Expression | Sense | 11 | ENSG00000- | APOC1 | 341 | apolipoprotein C-I | Chr | Forward | 305 |
| 8557- | probeset | (Fully | | 130208 | | | [Source: HGNC | 19 | Strand | |
| 31a_x_at | | Exonic) | | | | | Symbol; Acc: 607] | | | |
| OCMXSNG. | Expression | Sense | 11 | ENSG00000- | B2M | 567 | beta-2-microglobulin | Chr | Forward | 306 |
| 5067_s_at | probeset | (Fully | | 166710 | | | [Source: HGNC | 15 | Strand | |
| | | Exonic) | | | | | Symbol; Acc: 914] | | | |
| OCADNP. | Expression | Sense | 11 | ENSG00000- | B2M | 567 | beta-2-microglobulin | Chr | Forward | 307 |
| 3105_s_at | probeset | (includes | | 166710 | | | [Source: HGNC | 15 | Strand | |
| | | Intronic) | | | | | Symbol; Acc: 914] | | | |
| OC3SNG. | Expression | Sense | 11 | ENSG00000- | BST2 | 684 | bone marrow stromal cell | Chr | Forward | 308 |
| 1495- | probeset | (Fully | | 130303 | | | antigen 2 [Source: HGNC | 19 | Strand | |
| 79a_s_at | | Exonic) | | | | | Symbol; Acc: 1119] | | | |
| OC3SNGnh. | Expression | Sense | 7 | ENSG00000- | BST2 | 684 | bone marrow stromal cell | Chr | Forward | 309 |
| 10611_x_at | probeset | (Fully | | 130303 | | | antigen 2 [Source: HGNC | 19 | Strand | |
| | | Exonic) | | | | | Symbol; Acc: 1119] | | | |
| OC3P.1545. | Expression | Sense | 7 | ENSG00000- | BST2 | 684 | bone marrow stromal cell | Chr | Forward | 310 |
| CB1_x_at | probeset | (Fully | | 130303 | | | antigen 2 [Source: HGNC | 19 | Strand | |
| | | Exonic) | | | | | Symbol; Acc: 1119] | | | |
| OC3P.5468. | Expression | Sense | 11 | ENSG00000- | C1QB | 713 | complement component 1, | Chr | Forward | 311 |
| C1_s_at | probeset | (Fully | | 173369 | | | q subcomponent, B chain | 1 | Strand | |
| | | Exonic) | | | | | [Source: HGNC | | | |
| | | | | | | | Symbol; Acc: 1242] | | | |
| OCMXSNG. | Expression | Anti- | 11 | ENSG00000- | C1QC | 714 | complement component 1, | Chr | Forward | 312 |
| 5528_s_at | probeset | Sense | | 159189 | | | q subcomponent, C chain | 1 | Strand | |
| | | | | | | | [Source: HGNC | | | |
| | | | | | | | Symbol; Acc: 1245] | | | |
| OC3SNG. | Expression | Sense | 8 | ENSG00000- | C1QC | 714 | complement component 1, | Chr | Forward | 313 |
| 856- | probeset | (Fully | | 159189 | | | q subcomponent, C chain | 1 | Strand | |
| 35a_x_at | | Exonic) | | | | | [Source: HGNC | | | |
| | | | | | | | Symbol; Acc: 1245] | | | |
| OC3SNGn. | Expression | Sense | 7 | ENSG00000- | C1S | 716 | complement component 1, | Chr | Forward | 314 |
| 6006- | probeset | (Fully | | 182326 | | | q subcomponent, | 12 | Strand | |
| 1022a_s_at | | Exonic) | | | | | [Source: HGNC | | | |
| | | | | | | | Symbol; Acc: 1247] | | | |
| OCHP.887_ | Expression | Sense | 11 | ENSG00000- | CRB3 | 874 | carbonyl reductase 3 | Chr | Forward | 315 |
| s_at | probeset | (Fully | | 159231 | | | [Source: HGNC | 21 | Strand | |
| | | Exonic) | | | | | Symbol; Acc: 1549] | | | |
| OC3P.9251. | Expression | Sense | 11 | ENSG00000- | CD4 | 920 | CD4 molecule | Chr | Forward | 316 |
| C1_s_at | probeset | (Fully | | 010610 | | | [Source: HGNC | 12 | Strand | |
| | | Exonic) | | | | | Symbol; Acc: 1678] | | | |
| OC3P.4732. | Expression | Sense | 11 | ENSG00000- | CD44 | 960 | CD4 molecule | Chr | Forward | 317 |
| C1_s_at | probeset | (Fully | | 026508 | | | (Indian blood group) | 11 | Strand | |
| | | Exonic) | | | | | [Source: HGNC | | | |
| | | | | | | | Symbol; Acc: 1681] | | | |
| OCMX.670. | Expression | Anti- | 9 | ENSG00000- | CD74 | 972 | CD74 molecule, major | Chr | Reverse | 318 |
| CB2_at | probeset | Sense | | 019582 | | | histocompatibility complex, | 5 | Strand | |
| | | | | | | | class II invariant chain | | | |
| | | | | | | | [Source: HGNC | | | |
| | | | | | | | Symbol; Acc: 1697] | | | |
| OC3SNG. | Expression | Sense | 11 | ENSG00000- | CD74 | 972 | CD74 molecule, major | Chr | Reverse | 319 |
| 3064- | probeset | (Fully | | 019582 | | | histocompatibility complex, | 5 | Strand | |
| 21a_x_at | | Exonic) | | | | | class II invariant chain | | | |
| | | | | | | | [Source: HGNC | | | |
| | | | | | | | Symbol; Acc: 1697] | | | |
| OCHP.366_ | Expression | Sense | 11 | ENSG00000- | CTSB | 1508 | cathepsin B [Source: HGNC | Chr | Reverse | 320 |
| s_at | probeset | (Fully | | 164733 | | | Symbol; Acc: 2527] | 8 | Strand | |
| | | Exonic) | | | | | | | | |
| OC3P.77. | Expression | Sense | 11 | ENSG00000- | CTSB | 1508 | cathepsin B [Source: HGNC | Chr | Reverse | 321 |
| C1_s_at | probeset | (Fully | | 164733 | | | Symbol; Acc: 2527] | 8 | Strand | |
| | | Exonic) | | | | | | | | |
| OCMX. | Expression | Sense | 11 | ENSG00000- | CTSB | 1508 | cathepsin B [Source: HGNC | Chr | Reverse | 322 |
| 2432. | probeset | (Fully | | 164733 | | | Symbol; Acc: 2527] | 8 | Strand | |
| C4_s_at | | Exonic) | | | | | | | | |
| OCADNP. | Expression | Sense | 10 | ENSG00000- | CTSS | 1520 | cathepsin S [Source: HGNC | Chr | Reverse | 323 |
| 7474_s_at | probeset | (Fully | | 163131 | | | Symbol; Acc: 2545] | 1 | Strand | |
| | | Exonic) | | | | | | | | |
| OC3SNG. | Expression | Sense | 11 | ENSG00000- | CYLD | 1540 | cylindromatosis (turban | Chr | Forward | 324 |
| 3595- | probeset | (Fully | | 083799 | | | tumor syndrome) | 16 | Strand | |
| 3338a_s_at | | Exonic) | | | | | [Source: HGNC | | | |
| | | | | | | | Symbol; Acc: 2584] | | | |
| OCRS2. | Expression | Sense | 11 | ENSG00000- | DGKA | 1606 | diacylglycerol kinase, alpha | Chr | Forward | 325 |
| 2290_s_at | probeset | (Fully | | 065357 | | | 80 kDa [Source: HGNC | 12 | Strand | |
| | | Exonic) | | | | | Symbol; Acc: 2849] | | | |
| OC3SNG. | Expression | Sense | 11 | ENSG00000- | FBP1 | 2203 | fructose-1,6-bisphosphatase | Chr | Reverse | 326 |
| 2053- | probeset | (Fully | | 165140 | | | 1 [Source: HGNC | 9 | Strand | |
| 58a_s_at | | Exonic) | | | | | Symbol; Acc: 3606] | | | |
| C3SNGnh. | Expression | Sense | 11 | ENSG00000- | FCER1G | 2207 | Fc fragment of IgE, | Chr | Forward | 327 |
| 2550_s_at | probeset | (Fully | | 158869 | | | high affinity | 1 | Strand | |
| | | Exonic) | | | | | I, receptor for; gamma | | | |
| | | | | | | | polypeptide [Source: HGNC | | | |
| | | | | | | | Symbol; Acc: 3611] | | | |
| OC3P. | Expression | Sense | 9 | ENSG00000- | FCGR2A | 2212 | Fc fragment of IgE, | Chr | Forward | 328 |
| 4815. | probeset | (Fully | | 143226 | | | low affinity | 1 | Strand | |
| C1_s_at | | Exonic) | | | | | IIa, receptor (CD32) | | | |
| | | | | | | | [Source: HGNC | | | |
| | | | | | | | Symbol; Acc: 3616] | | | |
| OCMX.125. | Expression | Anti- | 10 | ENSG00000- | GBP1 | 2633 | guanylate binding protein 1, | Chr | Reverse | 329 |
| C1_s_at | probeset | Sense | | 117228 | | | interferon-inducible | 1 | Strand | |
| | | | | | | | [Source: HGNC | | | |
| | | | | | | | Symbol; Acc: 4182] | | | |
| OCADA. | Expression | Sense | 11 | ENSG00000- | GBP1 | 2633 | guanylate binding protein 1, | Chr | Reverse | 330 |
| 10565_s_at | probeset | (Fully | | 117228 | | | interferon-inducible | 1 | Strand | |
| | | Exonic) | | | | | [Source: HGNC | | | |
| | | | | | | | Symbol; Acc: 4182] | | | |
| OC3P.3169. | Expression | Sense | 11 | ENSG00000- | GBP2 | 2634 | guanylate binding protein 1, | Chr | Reverse | 331 |
| C1_s_at | probeset | (Fully | | 162645 | | | interferon-inducible | 1 | Strand | |
| | | Exonic) | | | | | [Source: HGNC | | | |
| | | | | | | | Symbol; Acc: 4183] | | | |
| OCHP. | Expression | Sense | 11 | ENSG00000- | GLRX | 2745 | glutaredoxin | Chr | Reverse | 332 |
| 1436_s_at | probeset | (Fully | | 173221 | | | (thioltransferase) | 5 | Strand | |
| | | Exonic) | | | | | [Source: HGNC | | | |
| | | | | | | | Symbol; Acc: 4330] | | | |
| OCHP. | Expression | Sense | 11 | ENSG00000- | SFN | 2810 | stratifin [Source: HGNC | Chr | Forward | 333 |
| 345_s_at | probeset | (Fully | | 175793 | | | Symbol; Acc: 10773] | 1 | Strand | |
| | | Exonic) | | | | | | | | |
| OC3P.5227. | Expression | Sense | 11 | ENSG00000- | HCLS1 | 3059 | hematopoietic | Chr | Reverse | 334 |
| C1_s_at | probeset | (Fully | | 180353 | | | cell-specific Lyn | 3 | Strand | |
| | | Exonic) | | | | | substrate 1 [Source: HGNC | | | |
| | | | | | | | Symbol; Acc: 4844] | | | |
| OCRS2. | Expression | Sense | 11 | ENSG00000- | HLA-A | 3059 | hematopoietic | Chr | Reverse | 335 |
| 2571_s_at | probeset | (Fully | | 180353 | | | cell-specific Lyn | 3 | Strand | |
| | | Exonic) | | | | | substrate 1 [Source: HGNC | | | |
| | | | | | | | Symbol; Acc: 4844] | | | |
| OC3SNGn. | Expression | Sense | 9 | ENSG00000- | HLA-A | 3105 | major histocompatibility | Chr | Forward | 336 |
| 6880-3480a_ | probeset | (Fully | | 206503 | | | complex, class I, A | 6 | Strand | |
| x_at | | Exonic) | | | | | [Source: HGNC | | | |
| | | | | | | | Symbol; Acc: 4931] | | | |
| OC3SNGn. | Expression | Sense | 11 | ENSG00000- | HLA-A | 3105 | major histocompatibility | Chr | Forward | 337 |
| 1244-62a_ | probeset | (Fully | | 206503 | | | complex, class I, A | 6 | Strand | |
| x_at | | Exonic) | | | | | [Source: HGNC | | | |
| | | | | | | | Symbol; Acc: 4931] | | | |
| OC3SNGn. | Expression | Sense | 9 | ENSG00000- | HLA-A | 3105 | major histocompatibility | Chr | Forward | 338 |
| 6460-38a_ | probeset | (Fully | | 206503 | | | complex, class I, A | 6 | Strand | |
| x_at | | Exonic) | | | | | [Source: HGNC | | | |
| | | | | | | | Symbol; Acc: 4931] | | | |
| OCRS2. | Expression | Sense | 11 | ENSG00000- | HLA-B | 3106 | major histocompatibility | Chr | Reverse | 339 |
| 731_x_at | probeset | (Fully | | 234745 | | | complex, class I, B | 6 | Strand | |
| | | Exonic) | | | | | [Source: HGNC | | | |
| | | | | | | | Symbol; Acc: 4932] | | | |
| OC3P.141. | Expression | Sense | 9 | ENSG00000- | HLA-B | 3106 | major histocompatibility | Chr | Reverse | 340 |
| C12_x_at | probeset | (Fully | | 234745 | | | complex, class I, B | 6 | Strand | |
| | | Exonic) | | | | | [Source: HGNC | | | |
| | | | | | | | Symbol; Acc: 4932] | | | |
| OC3P.4729. | Expression | Sense | 11 | ENSG00000- | HLA- | 3109 | major histocompatibility | Chr | Reverse | 341 |
| C1_s_at | probeset | (Fully | | 242574 | DMB | | complex, class II, DM beta | 6 | Strand | |
| | | Exonic) | | | | | [Source: HGNC | | | |
| | | | | | | | Symbol; Acc: 4935] | | | |
| OC3SNGn. | Expression | Sense | 11 | ENSG00000- | HLA- | 3113 | major histocompatibility | Chr | Reverse | 342 |
| 2735-12a_ | probeset | (Fully | | 231389 | DPA1 | | complex, class II, | 6 | Strand | |
| s_at | | Exonic) | | | | | DP alpha 1 | | | |
| | | | | | | | [Source: HGNC | | | |
| | | | | | | | Symbol; Acc: 4938] | | | |
| OC3P.1664. | Expression | Sense | 11 | ENSG00000- | HLA- | 3115 | major histocompatibility | Chr | Forward | 343 |
| C1_s_at | probeset | (Fully | | 223865 | DPA1 | | complex, class II, | 6 | Strand | |
| | | Exonic) | | | | | DP beta 1 | | | |
| | | | | | | | [Source: HGNC | | | |
| | | | | | | | Symbol; Acc: 4940] | | | |
| OC3P.141. | Expression | Sense | 11 | ENSG00000- | HLA- | 3134 | major histocompatibility | Chr | Forward | 344 |
| C13_s_at | probeset | (Fully | | 204642 | F | | complex, class I, F | 6 | Strand | |
| | | Exonic) | | | | | [Source: HGNC | | | |
| | | | | | | | Symbol; Acc: 4963] | | | |
| OCRS2. | Expression | Sense | 11 | ENSG00000- | HLA- | 3134 | major histocompatibility | Chr | Forward | 345 |
| 2819_x_at | probeset | (Fully | | 204642 | F | | complex, class I, F | 6 | Strand | |
| | | Exonic) | | | | | [Source: HGNC | | | |
| | | | | | | | Symbol; Acc: 4963] | | | |
| OCRS2. | Expression | Sense | 11 | ENSG00000- | HLA- | 3134 | major histocompatibility | Chr | Forward | 346 |
| 2819_at | probeset | (Fully | | 204642 | F | | complex, class I, F | 6 | Strand | |
| | | Exonic) | | | | | [Source: HGNC | | | |
| | | | | | | | Symbol; Acc: 4963] | | | |
| OC3P.2460. | Expression | Sense | 11 | ENSG00000- | IFIT2 | 3433 | interferon-induced protein | Chr | Forward | 347 |
| C1_s_at | probeset | (Fully | | 119922 | | | with tetratricopeptide | 10 | Strand | |
| | | Exonic) | | | | | repeats | | | |
| | | | | | | | 2 [Source: HGNC | | | |
| | | | | | | | Symbol; Acc: 5409] | | | |
| OCHP. | Expression | Sense | 11 | ENSG00000- | IL1RN | 3557 | interleukin 1 receptor | Chr | Forward | 348 |
| 489_s_at | probeset | (Fully | | 136689 | | | antagonsit [Source: HGNC | 2 | Strand | |
| | | Exonic) | | | | | Symbol; Acc: 6000] | | | |
| OC3P.4435. | Expression | Sense | 11 | ENSG00000- | IRF1 | 3659 | interferon regulatory factor | Chr | Reverse | 349 |
| C1_401a_ | probeset | (Fully | | 125347 | | | 1 [Source: HGNC | 5 | Strand | |
| s_at | | Exonic) | | | | | Symbol; Acc: 6116] | | | |
| OCRS2. | Expression | Sense | 11 | ENSG00000- | ITGB2 | 3689 | integrin, beta 2 (complement | Chr | Reverse | 350 |
| 4310_s_at | probeset | (Fully | | 160255 | | | component 3 | 21 | Strand | |
| | | Exonic) | | | | | receptor 3 and 4 subunit) | | | |
| | | | | | | | [Source: HGNC | | | |
| | | | | | | | Symbol; Acc: 6155] | | | |
| OC3P.8722. | Expression | Sense | 11 | ENSG00000- | ITGB2 | 3689 | integrin, beta 2 (complement | Chr | Reverse | 351 |
| C1_s_at | probeset | (Fully | | 160255 | | | component 3 receptor | 21 | Strand | |
| | | Exonic) | | | | | 3 and 4 subunit) | | | |
| | | | | | | | [Source: HGNC | | | |
| | | | | | | | Symbol; Acc: 6155] | | | |
| OC3P.1033. | Expression | Sense | 11 | ENSG00000- | LGAL59 | 3965 | lectin, galactoside-binding, | Chr | Forward | 352 |
| C1_s_at | probeset | (Fully | | 168961 | | | soluble, 9 [Source: HGNC | 17 | Strand | |
| | | Exonic) | | | | | Symbol; Acc: 6570] | | | |
| OC3P.5449. | Expression | Sense | 8 | ENSG00000- | LRMP | 4033 | lymphoid-restricted | Chr | Forward | 353 |
| C1_at | probeset | (Fully | | 118308 | | | membrane protein | 12 | Strand | |
| | | | | | | | [Source: HGNC | | | |
| | | Exonic) | | | | | Symbol; Acc: 6690] | | | |
| OCMX. | Expression | Anti- | 11 | ENSG00000- | LTB | 4050 | lymphotoxin beta (TNF | Chr | Reverse | 354 |
| 5583. | probeset | Sense | | 227507 | | | superfamily, member 3) | 6 | Strand | |
| C1_s_at | | | | | | | [Source: HGNC | | | |
| | | | | | | | Symbol; Acc: 6711] | | | |
| OC3P.805. | Expression | Sense | 11 | ENSG00000- | CIITA | 4261 | class II, major | Chr | Forward | 355 |
| C1_s_at | probeset | (Fully | | 179583 | | | histocompatibility complex, | 16 | Strand | |
| | | Exonic) | | | | | transactivator | | | |
| | | | | | | | [Source: HGNC | | | |
| | | | | | | | Symbol; Acc: 7067] | | | |
| OC3SNGn. | Expression | Sense | 10 | ENSG00000- | MMP7 | 4316 | matrix metallopeptidase 7 | Chr | Reverse | 356 |
| 5100- | probeset | (Fully | | 137673 | | | (matrilysin, uterine) | 11 | Strand | |
| 4676a_s_at | | Exonic) | | | | | [Source: HGNC | | | |
| | | | | | | | Symbol; Acc: 7174] | | | |
| OC3SNGnh. | Expression | Sense | 10 | ENSG00000- | MX1 | 4599 | myxovirus (influenza virus) | Chr | Forward | 357 |
| 19645_s_at | probeset | (Fully | | 157601 | | | resistance 1, interferon- | 21 | Strand | |
| | | Exonic) | | | | | inducible protein p78 | | | |
| | | | | | | | (mouse) | | | |
| | | | | | | | [Source: HGNC | | | |
| | | | | | | | Symbol; Acc: 7532] | | | |
| OCHP.1640_ | Expression | Sense | 11 | ENSG00000- | NNMT | 4837/// | nicotinamide N- | Chr | Forward | 358 |
| s_at | probeset | (Fully | | 166741 | | 101928916 | methyltransferase | 11 | Strand | |
| | | Exonic) | | | | | [Source: HGNC | | | |
| | | | | | | | Symbol; Acc: 7861] | | | |
| OC3P.6011. | Expression | Sense | 11 | ENSG00000- | PLCG2 | 5336 | phospholipase C, gamma 2 | Chr | Forward | 359 |
| C1_s_at | probeset | (Fully | | 197943 | | | (phosphatidylinositol- | 16 | Strand | |
| | | Exonic) | | | | | specific) | | | |
| | | | | | | | [Source: HGNC | | | |
| | | | | | | | Symbol; Acc: 9066] | | | |
| OCRS2. | Expression | Sense | 11 | ENSG00000- | PML | 5371 | promyelocytic leukemia | Chr | Forward | 360 |
| 4548_s_at | probeset | (Fully | | 140464 | | | [Source: HGNC | 15 | Strand | |
| | | Exonic) | | | | | Symbol; Acc: 9113] | | | |
| OC3SNG. | Expression | Sense | 11 | ENSG00000- | SAT1 | 6303 | spermidine/spermine N1- | Chr | Forward | 361 |
| 1855- | probeset | (Fully | | 130066 | | | acetyltransferase 1 | X | Strand | |
| 2142a_s_at | | Exonic) | | | | | [Source: HGNC | | | |
| | | | | | | | Symbol; Acc: 10540] | | | |
| OCHPRC. | Expression | Anti- | 9 | ENSG00000- | SAT1 | 6303 | spermidine/spermine N1- | Chr | Forward | 362 |
| 804_s_at | probeset | Sense | | 130066 | | | acetyltransferase 1 | X | Strand | |
| | | | | | | | [Source: HGNC | | | |
| | | | | | | | Symbol; Acc: 10540] | | | |
| OC3P. | Expression | Anti- | 11 | ENSG00000- | SAT1 | 6303 | spermidine/spermine N1- | Chr | Forward | 363 |
| 9355. | probeset | Sense | | 130066 | | | acetyltransferase 1 | X | Strand | |
| C1_s_at | | | | | | | [Source: HGNC | | | |
| | | | | | | | Symbol; Acc: 10540] | | | |
| OCHP. | Expression | Sense | 11 | ENSG00000- | SIGLEC1 | 6614 | sialic acid binding Ig-like | Chr | Reverse | 364 |
| 1827_s_at | probeset | (Fully | | 088827 | | | lectin 1, sialoadhesin | 20 | Strand | |
| | | Exonic) | | | | | [Source: HGNC | | | |
| | | | | | | | Symbol; Acc: 11127] | | | |
| OCHP. | Expression | Sense | 11 | ENSG00000- | STAT1 | 6772 | signal transducer and activator | Chr | Reverse | 365 |
| 1588_s_at | probeset | (Fully | | 115415 | | | of transcription 1, 91 kDa | 2 | Strand | |
| | | Exonic) | | | | | [Source: HGNC | | | |
| | | | | | | | Symbol; Acc: 11362] | | | |
| Adx-Hs- | Almac | Sense | 10 | ENSG00000- | STAT1 | 6772 | signal transducer and activator | Chr | Reverse | 366 |
| ISGF3A- | redesigned | (Fully | | 115415 | | | of transcription 1, 91 kDa | 2 | Strand | |
| 300- | standard | Exonic) | | | | | [Source: HGNC | | | |
| 3_x_at | human | | | | | | Symbol; Acc: 11362] | | | |
| | control | | | | | | | | | |
| Adx-Hs- | Almac | Sense | 9 | ENSG00000- | STAT1 | 6772 | signal transducer and activator | Chr | Reverse | 367 |
| ISGF3A- | redesigned | (Fully | | 115415 | | | of transcription 1, 91 kDa | 2 | Strand | |
| 400- | standard | Exonic) | | | | | [Source: HGNC | | | |
| 3_x_at | human | | | | | | Symbol; Acc: 11362] | | | |
| | control | | | | | | | | | |
| OCADNP. | Expression | Sense | 11 | ENSG00000- | STAT1 | 6772 | signal transducer and activator | Chr | Reverse | 368 |
| 3111_s_at | probeset | (includes | | 115415 | | | of transcription 1, 91 kDa | 2 | Strand | |
| | | Intronic) | | | | | [Source: HGNC | | | |
| | | | | | | | Symbol; Acc: 11362] | | | |
| OC3SNG. | Expression | Sense | 10 | ENSG00000- | TYROBP | 7305 | TYRO protein tyrosine | Chr | Reverse | 369 |
| 2984- | probeset | (Fully | | 011600 | | | kinase binding protein | 19 | Strand | |
| 24a_s_at | | Exonic) | | | | | [Source: HGNC | | | |
| | | | | | | | Symbol; Acc: 12449] | | | |
| OC3P.7284. | Expression | Sense | 11 | ENSG00000- | VCAM1 | 7412 | vascular cell | Chr | Forward | 370 |
| C1_s_at | probeset | (Fully | | 162692 | | | adhesion molecule | 1 | Strand | |
| | | Exonic) | | | | | [Source: HGNC | | | |
| | | | | | | | Symbol; Acc: 12663] | | | |
| OC3P.530. | Expression | Sense | 11 | ENSG00000- | XBP1 | 7494 | X-box binding protein 1 | Chr | Reverse | 371 |
| C1- | probeset | (Fully | | 100219 | | | [Source: HGNC | 22 | Strand | |
| 561a_s_at | | Exonic) | | | | | Symbol; Acc: 12801] | | | |
| OC3P.11519. | Expression | Sense | 11 | ENSG00000- | KCNAB2 | 8514 | potassium voltage-gated | Chr | Forward | 372 |
| C1_s_at | probeset | (Fully | | 069424 | | | channel, shaker-related | 1 | Strand | |
| | | Exonic) | | | | | subfamily, beta member 2 | | | |
| | | | | | | | [Source: HGNC | | | |
| | | | | | | | Symbol; Acc: 6229] | | | |
| OCMXSNG. | Expression | Anti- | 11 | ENSG00000- | APOL1 | 8542 | apolipoprotein L, 1 | Chr | Forward | 373 |
| 5608_at | probeset | Sense | | 100342 | | | [Source: HGNC | 22 | Strand | |
| | | | | | | | Symbol; Acc: 618] | | | |
| OC3P.1177. | Expression | Sense | 11 | ENSG00000- | APOL1 | 8542 | apolipoprotein L, 1 | Chr | Forward | 374 |
| C1_x_at | probeset | (Fully | | 100342 | | | [Source: HGNC | 22 | Strand | |
| | | Exonic) | | | | | Symbol; Acc: 618] | | | |
| OC3P.7068. | Expression | Sense | 11 | ENSG00000- | UBE2L6 | 9246 | ubiquitin-conjugating enzyme | Chr | Reverse | 375 |
| C1_s_at | probeset | (Fully | | 156587 | | | E2L 6 [Source: HGNC | 11 | Strand | |
| | | Exonic) | | | | | Symbol; Acc: 12490] | | | |
| OC3SNGn. | Expression | Sense | 11 | ENSG00000- | CD163 | 9332 | CD163 molecule | Chr | Reverse | 376 |
| 2005- | probeset | (Fully | | 177575 | | | [Source: HGNC | 12 | Strand | |
| 402a_s_at | | Exonic) | | | | | Symbol; Acc: 1631] | | | |
| OC3P.5930. | Expression | Sense | 11 | ENSG00000- | UTAF | 9516 | lipopolysaccharide-induced | Chr | Reverse | 377 |
| C1_at | probeset | (Fully | | 189067 | | | TNF factor [Source: HGNC | 16 | Strand | |
| | | Exonic) | | | | | Symbol; Acc: 16841] | | | |
| OC3P.9869. | Expression | Sense | 11 | ENSG00000- | MAFB | 9935 | v-maf avian | Chr | Reverse | 378 |
| C1_s_at | probeset | (Fully | | 204103 | | | musculoapon eurotic | 20 | Strand | |
| | | Exonic) | | | | | fibrosarcoma oncogene | | | |
| | | | | | | | homolog B [Source: HGNC | | | |
| | | | | | | | Symbol; Acc: 6408] | | | |
| OC3SNG. | Expression | Sense | 10 | ENSG00000- | RASGRP2 | 10235 | RAS guanyl releasing | Chr | Reverse | 379 |
| 4002- | probeset | (Fully | | 068831 | | | protein 2 | 11 | Strand | |
| 20a_s_at | | Exonic) | | | | | (calcium and | | | |
| | | | | | | | DAG-regulated) | | | |
| | | | | | | | [Source: HGNC | | | |
| | | | | | | | Symbol; Acc: 9879] | | | |
| OC3P.616. | Expression | Sense | 11 | ENSG00000- | GPNMB | 10457 | glycoprotein | Chr | Forward | 380 |
| C1_s_at | probeset | (Fully | | 136235 | | | (transmembrane) | 7 | Strand | |
| | | Exonic) | | | | | nmb [Source: HGNC | | | |
| | | | | | | | Symbol; Acc: 4462] | | | |
| OC3P.13391. | Expression | Sense | 11 | ENSG00000- | ARID5A | 10865 | AT rich interactive | Chr | Forward | 381 |
| C1_s_at | probeset | (Fully | | 196843 | | | domain 5A (MRF1-like) | 2 | Strand | |
| | | Exonic) | | | | | [Source: HGNC | | | |
| | | | | | | | Symbol; Acc: 17361] | | | |
| OC3P.13144. | Expression | Sense | 11 | ENSG00000- | HMHA1 | 23526 | histocompatibility (minor) | Chr | Forward | 382 |
| C1-468a_ | probeset | (Fully | | 180448 | | | HA-1 [Source: HGNC | 19 | Strand | |
| s_at | | Exonic) | | | | | Symbol; Acc: 17102] | | | |
| OC3SNGn. | Expression | Sense | 11 | ENSG00000- | IFIT5 | 24138 | interferon-induced protein | Chr | Forward | 383 |
| 1535- | probeset | (Fully | | 152778 | | | with tetratricopeptide repeats | 10 | Strand | |
| 1076a_s_at | | Exonic) | | | | | 5 [Source: HGNC | | | |
| | | | | | | | Symbol; Acc: 13328] | | | |
| OC3P.11092. | Expression | Sense | 11 | ENSG00000- | CCDC69 | 26112 | coiled-coil domain | Chr | Reverse | 384 |
| C1_s_at | probeset | (Fully | | 198624 | | | containing | 5 | Strand | |
| | | Exonic) | | | | | 69 [Source: HGNC | | | |
| | | | | | | | Symbol; Acc: 24487] | | | |
| OC3P.2293. | Expression | Sense | 9 | ENSG00000- | NUPR1 | 26471 | nuclear protein, | Chr | Reverse | 385 |
| C2_x_at | probeset | (Fully | | 176046 | | | transcriptional | 16 | Strand | |
| | | Exonic) | | | | | regulator, 1 | | | |
| | | | | | | | [Source: HGNC | | | |
| | | | | | | | Symbol; Acc: 29990] | | | |
| OC3SNGn. | Expression | Sense | 9 | ENSG00000- | TREM2 | 54209 | triggering receptor | Chr | Reverse | 386 |
| 8835a_s_at | probeset | (Fully | | 095970 | | | expressed | 6 | Strand | |
| | | Exonic) | | | | | on myeloid cells 2 | | | |
| | | | | | | | [Source: HGNC | | | |
| | | | | | | | Symbol; Acc: 17761] | | | |
| OC3SNGnh. | Expression | Sense | 11 | ENSG00000- | PARP14 | 54625 | poly (ADP-ribose) | Chr | Forward | 387 |
| 2575_s_at | probeset | (includes | | 173193 | | | polymerase | 3 | Strand | |
| | | Intronic) | | | | | family, member 14 | | | |
| | | | | | | | [Source: HGNC | | | |
| | | | | | | | Symbol; Acc: 29232] | | | |
| OC3SNGn. | Expression | Sense | 11 | ENSG00000- | XAF1 | 54739 | XIAP associated factor 1 | Chr | Forward | 388 |
| 2605- | probeset | (Fully | | 132530 | | | [Source: HGNC | 17 | Strand | |
| 236a_x_at | | Exonic) | | | | | Symbol; Acc: 30932] | | | |
| OC3P.4873. | Expression | Sense | 11 | ENSG00000- | XAF1 | 54739 | XIAP associated factor 1 | Chr | Forward | 389 |
| C1_s_at | probeset | (Fully | | 132530 | | | [Source: HGNC | 17 | Strand | |
| | | Exonic) | | | | | Symbol; Acc: 30932] | | | |
| OC3P.8320. | Expression | Sense | 11 | ENSG00000- | FAM20A | 54757 | family with sequence | Chr | Reverse | 390 |
| C1_s_at | probeset | (Fully | | 108950 | | | similarity 20, member A | 17 | Strand | |
| | | Exonic) | | | | | [Source: HGNC | | | |
| | | | | | | | Symbol; Acc: 23015] | | | |
| OC3P.8314. | Expression | Sense | 11 | ENSG00000- | SAMD9 | 54809 | sterile alpha motif domain | Chr | Reverse | 391 |
| C1_s_at | probeset | (Fully | | 205413 | | | containing 9 [Source: HGNC | 7 | Strand | |
| | | Exonic) | | | | | Symbol; Acc: 1348] | | | |
| OC3P.6316. | Expression | Sense | 11 | ENSG00000- | FAM46C | 54855 | family with sequence | Chr | Forward | 392 |
| C1_s_at | probeset | (Fully | | 183508 | | | similarity 46, member C | 1 | Strand | |
| | | Exonic) | | | | | [Source: HGNC | | | |
| | | | | | | | Symbol; Acc: 24712] | | | |
| OCADA. | Expression | Sense | 11 | ENSG00000- | FAM46C | 54855 | family with sequence | Chr | Forward | 393 |
| 10345_s_at | probeset | (Fully | | 183508 | | | similarity 46, member C | 1 | Strand | |
| | | Exonic) | | | | | [Source: HGNC | | | |
| | | | | | | | Symbol; Acc: 24712] | | | |
| OC3SNGn. | Expression | Sense | 11 | ENSG00000- | FLVCR2 | 55640 | feline leukemia virus | Chr | Forward | 394 |
| 2106- | probeset | (Fully | | 119686 | | | subgroup | 14 | Strand | |
| 17a_s_at | | Exonic) | | | | | C cellular receptor | | | |
| | | | | | | | family, member 2 | | | |
| | | | | | | | [Source: HGNC | | | |
| | | | | | | | Symbol; Acc: 20105] | | | |
| OCADNP. | Expression | Sense | 11 | ENSG00000- | AKiP1 | 56672 | A kinase (PRKA) interacting | Chr | Forward | 395 |
| 5178_s_at | probeset | (Fully | | 166452 | | | protein 1 [Source: HGNC | 11 | Strand | |
| | | Exonic) | | | | | Symbol; Acc: 1170] | | | |
| OCADA.1 | Expression | Sense | 8 | ENSG00000- | SLAMF7 | 57823 | SLAM family member 7 | Chr | Forward | 396 |
| 0811_s_at | probeset | (Fully | | 026751 | | | [Source: HGNC | 1 | Strand | |
| | | Exonic) | | | | | Symbol; Acc: 21394] | | | |
| OC3P. | Expression | Sense | 11 | ENSG00000- | IFIH1 | 64135 | interferon induced with | Chr | Reverse | 397 |
| 10280. | probeset | (Fully | | 115267 | | | helicase C domain 1 | 2 | Strand | |
| C1_s_at | | Exonic) | | | | | [Source: HGNC | | | |
| | | | | | | | Symbol; Acc: 18873] | | | |
| OCRS. | Expression | Sense | 11 | ENSG00000- | GAL3ST4 | 79690 | galactose-3-O- | Chr | Reverse | 398 |
| 727_s_at | probeset | (Fully | | 197093 | | | sulfotransferase 4 | 7 | Strand | |
| | | Exonic) | | | | | [Source: HGNC | | | |
| | | | | | | | Symbol; Acc: 24145] | | | |
| OC3P. | Expression | Sense | 9 | ENSG00000- | NLRC5 | 84166 | NLR family, CARD domain | Chr | Forward | 399 |
| 7557. | probeset | (Fully | | 140853 | | | containing 5 [Source: HGNC | 16 | Strand | |
| C1_s_at | | Exonic) | | | | | Symbol; Acc: 29933] | | | |
| OCADA. | Expression | Sense | 11 | ENSG00000- | DERL3 | 91319 | derlin 3 [Source: HGNC | Chr | Reverse | 400 |
| 3339_s_at | probeset | (Fully | | 099958 | | | Symbol; Acc: 14236] | 22 | Strand | |
| | | Exonic) | | | | | | | | |
| OC3SNGn. | Expression | Sense | 11 | ENSG00000- | GBP5 | 115362 | guanylate binding protein 5 | Chr | Reverse | 401 |
| 3058- | probeset | (Fully | | 154451 | | | [Source: HGNC | 1 | Strand | |
| 31a_s_at | | Exonic) | | | | | Symbol; Acc: 19895] | | | |
| OCMXSNG. | Expression | Anti- | 11 | ENSG00000- | BATF2 | 116071 | basic leucine zipper | Chr | Reverse | 402 |
| 2584_s_at | probeset | Sense | | 168062 | | | transcription factor, | 11 | Strand | |
| | | | | | | | ATF-like 2 | | | |
| | | | | | | | [Source: HGNC | | | |
| | | | | | | | Symbol; Acc: 25163] | | | |
| OCADA. | Expression | Sense | 11 | ENSG00000- | EMB | 133418 | embigin [Source: HGNC | Chr | Reverse | 403 |
| 8743_s_at | probeset | (Fully | | 170571 | | | Symbol; Acc: 30465] | 5 | Strand | |
| | | Exonic) | | | | | | | | |
| OCMXSNG. | Expression | Anti- | 11 | ENSG00000- | SAMD9L | 219285 | sterile alpha motif domain | Chr | Reverse | 404 |
| 448_s_at | probeset | Sense | | 177409 | | | containing 9-like | 7 | Strand | |
| | | | | | | | [Source: HGNC | | | |
| | | | | | | | Symbol; Acc: 1349] | | | |
| OC3P. | Expression | Sense | 11 | ENSG00000- | SAMD9L | 219285 | sterile alpha motif domain | Chr | Reverse | 405 |
| 10487. | probeset | (Fully | | 177409 | | | containing 9-like | 7 | Strand | |
| C1_s_at | | Exonic) | | | | | [Source: HGNC | | | |
| | | | | | | | Symbol; Acc: 1349] | | | |
| OC3P. | Expression | Sense | 11 | ENSG00000- | IL4I1 | 259308 | interleukin 4 induced 1 | Chr | Reverse | 406 |
| 969. | probeset | (Fully | | 104951 | | | [Source: HGNC | 19 | Strand | |
| C1_s_at | | Exonic) | | | | | Symbol; Acc: 19094] | | | |
| OCADA. | Expression | Sense | 11 | ENSG00000- | ODF3B | 440836 | outer dense fiber of | Chr | Reverse | 407 |
| 5772_s_at | probeset | (Fully | | 177989 | | | sperm tails 3B | 22 | Strand | |
| | | Exonic) | | | | | [Source: HGNC | | | |
| | | | | | | | Symbol; Acc: 34388] | | | |
| OCMXSNG. | Expression | Anti- | 11 | ENSG00000- | ODF3B | 440836 | outer dense fiber of | Chr | Reverse | 408 |
| 5626_s_at | probeset | Sense | | 177989 | | | sperm tails 3B | 22 | Strand | |
| | | | | | | | [Source: HGNC | | | |
| | | | | | | | Symbol; Acc: 34388] | | | |
| OCADA. | Expression | Sense | 11 | ENSG00000- | FAM26F | 441168 | family with sequence | Chr | Forward | 409 |
| 8654_s_at | probeset | (Fully | | 188820 | | | similarity 26, member F | 6 | Strand | |
| | | Exonic) | | | | | [Source: HGNC | | | |
| | | | | | | | Symbol; Acc: 33391] | | | |
| OC3P. | Expression | Sense | 11 | ENSG00000- | SOD2 | 6648/// | superoxide dismutase 2, | Chr | Reverse | 410 |
| 14483. | probeset | (Fully | | 112096 | | 100129518 | mitochondrial | 6 | Strand | |
| C1_s_at | | Exonic) | | | | | [Source: HGNC | | | |
| | | | | | | | Symbol; Acc: 11180] | | | |
| OCMX. | Expression | Sense | 11 | ENSG00000- | IFI44L | 10964 | interferon-induced protein | Chr | Forward | 411 |
| 15525. | probeset | (Fully | | 137959 | | | 44-like | 1 | Strand | |
| C1_s_at | | Exonic) | | | | | [Source: HGNC | | | |
| | | | | | | | Symbol; Acc: 17817] | | | |
| OC3P. | Expression | Sense | 10 | ENSG00000- | IFI44L | 10964 | interferon-induced protein | Chr | Forward | 412 |
| 6903.C1_at | probeset | (Fully | | 137959 | | | 44-like | 1 | Strand | |
| | | Exonic) | | | | | [Source: HGNC | | | |
| | | | | | | | Symbol; Acc: 17817] | | | |
| OC3SNG. | Expression | Sense | 10 | ENSG00000- | APOL6 | 80830 | apolipoprotein L, 6 | Chr | Forward | 413 |
| 3004- | probeset | (Fully | | 221963 | | | [Source: HGNC | 22 | Strand | |
| 20a_s_at | | Exonic) | | | | | Symbol; Acc: 14870] | | | |
| OCMXSNG. | Expression | Sense | 11 | ENSG00000- | N/A | N/A | NOVEL antisense | Chr | Forward | 414 |
| 1782_at | probeset | (includes | | 236449 | | | (Clone_based_vega_gene) | 2 | Strand | |
| | | Intronic) | | | | | | | | |
| OC3SNGn. | Expression | Insu- | 0 | 0 | 0 | 0 | 0 | 0 | 0 | 415 |
| 6005- | probeset | fficient | | | | | | | | |
| 4a_x_at | | probes | | | | | | | | |
| | | (<6) | | | | | | | | |
| OC3P. | Expression | Insu- | 0 | 0 | 0 | 0 | 0 | 0 | 0 | 416 |
| 264. | probeset | fficient | | | | | | | | |
| C16_x_at | | probes | | | | | | | | |
| | | (<6) | | | | | | | | |
| OC3SNG. | Expression | Insu- | 0 | 0 | 0 | 0 | 0 | 0 | 0 | 417 |
| 1434- | probeset | fficient | | | | | | | | |
| 27a_x_at | | probes | | | | | | | | |
| | | (<6) | | | | | | | | |
| OC3P. | Expression | Insu- | 0 | 0 | 0 | 0 | 0 | 0 | 0 | 418 |
| 264. | probeset | fficient | | | | | | | | |
| C2_x_at | | probes | | | | | | | | |
| | | (<6) | | | | | | | | |
| OC3P. | Expression | Insu- | 0 | 0 | 0 | 0 | 0 | 0 | 0 | 419 |
| 264. | probeset | fficient | | | | | | | | |
| CB1_x_at | | probes | | | | | | | | |
| | | (<6) | | | | | | | | |
| OC3P. | Expression | Insu- | 0 | 0 | 0 | 0 | 0 | 0 | 0 | 420 |
| 264. | probeset | fficient | | | | | | | | |
| CB5_x_at | | probes | | | | | | | | |
| | | (<6) | | | | | | | | |
| OC3SNGn. | Expression | No | 0 | 0 | 0 | 0 | 0 | 0 | 0 | 421 |
| 5999- | probeset | Ge- | | | | | | | | |
| 435a_x_at | | nome | | | | | | | | |
| | | match | | | | | | | | |
| OC3P. | Expression | Insu- | 0 | 0 | 0 | 0 | 0 | 0 | 0 | 422 |
| 264. | probeset | fficient | | | | | | | | |
| C10_x_at | | probes | | | | | | | | |
| | | (<6) | | | | | | | | |
| OC3P. | Expression | Insu- | 0 | 0 | 0 | 0 | 0 | 0 | 0 | 423 |
| 42. | probeset | fficient | | | | | | | | |
| CB2_x_at | | probes | | | | | | | | |
| | | (<6) | | | | | | | | |
| OCADNP. | Expression | Insu- | 0 | 0 | 0 | 0 | 0 | 0 | 0 | 424 |
| 9529_ | probeset | fficient | | | | | | | | |
| x_at | | probes | | | | | | | | |
| | | (<6) | | | | | | | | |
| OC3P. | Expression | Insu- | 0 | 0 | 0 | 0 | 0 | 0 | 0 | 425 |
| 121. | probeset | fficient | | | | | | | | |
| C9_x_at | | probes | | | | | | | | |
| | | (<6) | | | | | | | | |
| OCADNP. | Expression | Insu- | 0 | 0 | 0 | 0 | 0 | 0 | 0 | 426 |
| 6175_ | probeset | fficient | | | | | | | | |
| x_at | | probes | | | | | | | | |
| | | (<6) | | | | | | | | |
| OC3P. | Expression | Sense | 11 | ENSG00000- | IGLC3 | N/A | immunoglobulin lambda | Chr | Forward | 427 |
| 264. | probeset | (Fully | | 211679 | | | constant 3 (Kern-Oz+ | 22 | Strand | |
| C21_x_at | | Exonic) | | | | | marker) | | | |
| | | | | | | | [Source: HGNC | | | |
| | | | | | | | Symbol; Acc: 5857] | | | |
| OC3SNG. | Expression | Sense | 8 | ENSG00000- | IGLC3 | N/A | immunoglobulin lambda | Chr | Forward | 428 |
| 1849- | probeset | (Fully | | 211679 | | | constant 3 (Kern-Oz+ | 22 | Strand | |
| 16a_x_at | | Exonic) | | | | | marker) | | | |
| | | | | | | | [Source: HGNC | | | |
| | | | | | | | Symbol; Acc: 5857] | | | |
| OC3SNG. | Expression | Sense | 11 | ENSG00000- | IGLC3 | N/A | immunoglobulin lambda | Chr | Forward | 429 |
| 1049- | probeset | (Fully | | 211679 | | | constant 3 (Kern-Oz+ | 22 | Strand | |
| 17a_x_at | | Exonic) | | | | | marker) | | | |
| | | | | | | | [Source: HGNC | | | |
| | | | | | | | Symbol; Acc: 5857] | | | |
| OCMX. | Expression | Sense | 11 | ENSG00000- | N/A | N/A | KNOWN lincRNA | Chr | Reverse | 430 |
| 2630. | probeset | (Fully | | 253364 | | | (Clone_based_vega_gene) | 14 | Strand | |
| C25_s_at | | Exonic) | | | | | | | | |
| OC3SNGn. | Expression | Sense | 11 | ENSG00000- | IGHG1 | 101930374/// | immunoglobulin heavy | Chr | Reverse | 431 |
| 7538- | probeset | (Fully | | 211896 | | 102465871/// | constant gamma 1 (G1m | 14 | Strand | |
| 8a_s_at | | Exonic) | | | | 102466889 | marker) [Source: HGNC | | | |
| | | | | | | | Symbol; Acc: 5525] | | | |
| OCADNP. | Expression | Anti- | 11 | ENSG00000- | N/A | N/A | NOVEL miRNA | Chr | Forward | 432 |
| 6338_x_at | probeset | Sense | | 207835 | | | (Clone_based_ensembl_ | 22 | Strand | |
| | | | | | | | gene) | | | |
| OCMX. | Expression | Sense | 11 | ENSG00000- | N/A | N/A | KNOWN lincRNA | Chr | Reverse | 433 |
| 2630. | probeset | (Fully | | 253364 | | | (Clone_based_vega_gene) | 14 | Strand | |
| C11_s_at | | Exonic) | | | | | | | | |
| OC3P. | Expression | Sense | 6 | ENSG00000- | IGKC | N/A | immunoglobulin kappa | Chr | Reverse | 434 |
| 121. | probeset | (Fully | | 211592 | | | constant [Source: HGNC | 2 | Strand | |
| CB2_x_at | | Exonic) | | | | | Symbol; Acc: 5716] | | | |
| OC3SNGn. | Expression | Sense | 6 | ENSG00000- | IGKC | N/A | immunoglobulin kappa | Chr | Reverse | 435 |
| 6560- | probeset | (Fully | | 211592 | | | constant [Source: HGNC | 2 | Strand | |
| 270a_x_at | | Exonic) | | | | | Symbol; Acc: 5716] | | | |
| OC3P. | Expression | Sense | 11 | ENSG00000- | N/A | N/A | KNOWN pseudogene | Chr | Reverse | 436 |
| 10347. | probeset | (Fully | | 255594 | | | (Clone_based_ensembl_ | 22 | Strand | |
| C1_s_at | | Exonic) | | | | | gene) | | | |
| OC3P. | Expression | Sense | 11 | ENSG00000- | FCGR1B | 2209/// | Fc fragment of IgG, high | Chr | Reverse | 437 |
| 13221. | probeset | (Fully | | 198019 | | 2210/// | affinity Ib, receptor | 1 | Strand | |
| C1_s_at | | Exonic) | | | | 100132417 | (CD64) [Source: HGNC | | | |
| | | | | | | | Symbol; Acc: 3614] | | | |
| OCMXSNG. | Expression | Sense | 11 | ENSG00000- | RPL23- | N/A | ribosomal protein L23a | Chr | Reverse | 438 |
| 5045_s_at | probeset | (Fully | | 239257 | AP1 | | pseudogene 1 [Source: | 6 | Strand | |
| | | Exonic) | | | | | HGNC Symbol; | | | |
| | | | | | | | Acc: 10318] | | | |
| OCHP.1216_ | Expression | Sense | 11 | ENSG00000- | ACTA2 | 59 | actin, alpha 2, smooth | Chr | Reverse | 439 |
| s_at | probeset | (Fully | | 107796 | | | muscle aorta | 10 | Strand | |
| | | | | | | | [Source: HGNC | | | |
| | | Exonic) | | | | | Symbol; Acc: 130] | | | |
| OC3P. | Expression | Sense | 10 | ENSG00000- | ADH5 | 128 | alcohol dehydrogenase 5 | Chr | Reverse | 440 |
| 12692. | probeset | (Fully | | 197894 | | | (class III), chi polypeptide | 4 | Strand | |
| C1_s_at | | Exonic) | | | | | [Source: HGNC | | | |
| | | | | | | | Symbol; Acc: 253] | | | |
| OC3P. | Expression | Sense | 11 | ENSG00000- | AEBP1 | 165 | AE binding protein 1 | Chr | Forward | 441 |
| 3458. | probeset | (Fully | | 106624 | | | [Source: HGNC | 7 | Strand | |
| C1_s_at | | Exonic) | | | | | Symbol; Acc: 303] | | | |
| OC3SNGnh. | Expression | Sense | 10 | ENSG00000- | APBB2 | 323 | amyloid beta (A4) precursor | Chr | Reverse | 442 |
| 5051_at | probeset | (includes | | 163697 | | | protein-binding, family B, | 4 | Strand | |
| | | Intronic) | | | | | member 2 [Source: HGNC | | | |
| | | | | | | | Symbol; Acc: 582] | | | |
| OC3SNGnh. | Expression | Sense | 10 | ENSG00000- | APBB2 | 323 | amyloid beta (A4) precursor | Chr | Reverse | 443 |
| 5051_x_at | probeset | (includes | | 163697 | | | protein-binding, family B, | 4 | Strand | |
| | | Intronic) | | | | | member 2 [Source: HGNC | | | |
| | | | | | | | Symbol; Acc: 582] | | | |
| OCHP.1016_ | Expression | Sense | 11 | ENSG00000- | APOD | 347 | apolipoprotein D | Chr | Reverse | 444 |
| s_at | probeset | (Fully | | 189058 | | | [Source: HGNC | 3 | Strand | |
| | | Exonic) | | | | | Symbol; Acc: 612] | | | |
| OC3P.373. | Expression | Sense | 11 | ENSG00000- | RHOB | 388 | ras homolog family | Chr | Forward | 445 |
| C1- | probeset | (Fully | | 143878 | | | member B [Source: HGNC | 2 | Strand | |
| 533a_s_at | | Exonic) | | | | | Symbol; Acc: 668] | | | |
| OCMX. | Expression | Anti- | 8 | ENSG00000- | ATF3 | 467 | activating transcription | Chr | Forward | 446 |
| 4359. | probeset | Sense | | 162772 | | | factor 3 [Source: HGNC | 1 | Strand | |
| C1_x_at | | | | | | | Symbol; Acc: 785] | | | |
| OC3P.2321. | Expression | Sense | 11 | ENSG00000- | ATF3 | 467 | activating transcription | Chr | Forward | 447 |
| C1_s_at | probeset | (Fully | | 162772 | | | factor 3 [Source: HGNC | 1 | Strand | |
| | | Exonic) | | | | | Symbol; Acc: 785] | | | |
| OCMX. | Expression | Anti- | 11 | ENSG00000- | ATF3 | 467 | activating transcription | Chr | Forward | 448 |
| 4359.C1_at | probeset | Sense | | 162772 | | | factor 3 [Source: HGNC | 1 | Strand | |
| | | | | | | | Symbol; Acc: 785] | | | |
| OCADNP. | Expression | Sense | 11 | ENSG00000- | CA3 | 761 | carbonic anhydrase III, | Chr | Forward | 449 |
| 7610_s_at | probeset | (Fully | | 164879 | | | muscle specific | 8 | Strand | |
| | | Exonic) | | | | | [Source: HGNC | | | |
| | | | | | | | Symbol; Acc: 1374] | | | |
| OC3SNGnh. | Expression | Sense | 11 | ENSG00000- | CA3 | 761 | carbonic anhydrase III, | Chr | Forward | 450 |
| 4098_at | probeset | (includes | | 164879 | | | muscle specific | 8 | Strand | |
| | | Intronic) | | | | | [Source: HGNC | | | |
| | | | | | | | Symbol; Acc: 1374] | | | |
| OC3P.11093. | Expression | Sense | 9 | ENSG00000- | Ca12 | 771 | carbonic anhydrase XII | Chr | Reverse | 451 |
| C1_s_at | probeset | (Fully | | 074410 | | | [Source: HGNC | 14 | Strand | |
| | | Exonic) | | | | | Symbol; Acc: 1371] | | | |
| OCMX. | Expression | Anti- | 11 | ENSG00000- | CALD1 | 800 | caldesmon 1 | Chr | Forward | 452 |
| 517.C1_at | probeset | Sense | | 122786 | | | [Source: HGNC | 7 | Strand | |
| | | | | | | | Symbol; Acc: 1441] | | | |
| OCMX. | Expression | Anti- | 11 | ENSG00000- | CALD1 | 800 | caldesmon 1 | Chr | Forward | 453 |
| 517. | probeset | Sense | | 122786 | | | [Source: HGNC | 7 | Strand | |
| C1_x_at | | | | | | | Symbol; Acc: 1441] | | | |
| OCADNP. | Expression | Sense | 11 | ENSG00000- | CALD1 | 800 | caldesmon 1 | Chr | Forward | 454 |
| 2089_s_at | probeset | (Fully | | 122786 | | | [Source: HGNC | 7 | Strand | |
| | | Exonic) | | | | | Symbol; Acc: 1441] | | | |
| OCHP. | Expression | Sense | 11 | ENSG00000- | CDH11 | 1009 | cadherin 11, type 2, OB- | Chr | Reverse | 455 |
| 148_s_at | probeset | (Fully | | 140937 | | | cadherin (osteoblast) | 14 | Strand | |
| | | Exonic) | | | | | [Source: HGNC | | | |
| | | | | | | | Symbol; Acc: 1750] | | | |
| OC3SNGnh. | Expression | Sense | 11 | ENSG00000- | CHN1 | 1123 | chimerin 1 [Source: HGNC | Chr | Reverse | 456 |
| 3154_s_at | probeset | (Fully | | 128656 | | | Symbol; Acc: 1943] | 2 | Strand | |
| | | Exonic) | | | | | | | | |
| OC3P. | Expression | Anti- | 11 | ENSG00000- | CLNS1A | 1207 | chloride channel, | Chr | Reverse | 457 |
| 1738. | probeset | Sense | | 074201 | | | nucleotide-sensitive, 1A | 11 | Strand | |
| C3_x_at | | | | | | | [Source: HGNC | | | |
| | | | | | | | Symbol; Acc: 2080] | | | |
| OC3P. | Expression | Anti- | 11 | ENSG00000- | CLNS1A | 1207 | chloride channel, | Chr | Reverse | 458 |
| 1738. | probeset | Sense | | 074201 | | | nucleotide-sensitive, 1A | 11 | Strand | |
| | | | | | | | [Source: HGNC | | | |
| C3_at | | | | | | | Symbol; Acc: 2080] | | | |
| OC3P. | Expression | Sense | 11 | ENSG00000- | COL1A1 | 1277 | collagen, type I, alpha 1 | Chr | Reverse | 459 |
| 354. | probeset | (Fully | | 108821 | | | [Source: HGNC | 17 | Strand | |
| CB1_s_at | | Exonic) | | | | | Symbol; Acc: 2197] | | | |
| OCMXSNG. | Expression | Anti- | 11 | ENSG00000- | COL1A1 | 1277 | collagen, type I, alpha 1 | Chr | Reverse | 460 |
| 5132_s_at | probeset | Sense | | 108821 | | | [Source: HGNC | 17 | Strand | |
| | | | | | | | Symbol; Acc: 2197] | | | |
| OCMXSNG. | Expression | Anti- | 11 | ENSG00000- | COL1A1 | 1277 | collagen, type I, alpha 1 | Chr | Reverse | 461 |
| 5132_x_at | probeset | Sense | | 108821 | | | [Source: HGNC | 17 | Strand | |
| | | | | | | | Symbol; Acc: 2197] | | | |
| OC3SNGn. | Expression | Sense | 11 | ENSG00000- | COL1A2 | 1278 | collagen, type I, alpha 2 | Chr | Forward | 462 |
| 2538- | probeset | (Fully | | 164692 | | | [Source: HGNC | 7 | Strand | |
| 539a_x_at | | Exonic) | | | | | Symbol; Acc: 2198] | | | |
| OC3SNGn. | Expression | Sense | 9 | ENSG00000- | COL1A2 | 1278 | collagen, type I, alpha 2 | Chr | Forward | 463 |
| 8474- | probeset | (Fully | | 164692 | | | [Source: HGNC | 7 | Strand | |
| 50a_x_at | | Exonic) | | | | | Symbol; Acc: 2198] | | | |
| OC3SNGn. | Expression | Sense | 11 | ENSG00000- | COL3A1 | 1281 | collagen, type III, alpha 1 | Chr | Forward | 464 |
| 1211- | probeset | (Fully | | 168542 | | | [Source: HGNC | 2 | Strand | |
| 6a_s_at | | Exonic) | | | | | Symbol; Acc: 2201] | | | |
| OC3P.81. | Expression | Sense | 11 | ENSG00000- | COL3A1 | 1281 | collagen, type III, alpha 1 | Chr | Forward | 465 |
| CB2_s_at | probeset | (Fully | | 168542 | | | [Source: HGNC | 2 | Strand | |
| | | Exonic) | | | | | Symbol; Acc: 2201] | | | |
| OC3P.850. | Expression | Sense | 11 | ENSG00000- | COL4A1 | 1282 | collagen, type IV, alpha 1 | Chr | Reverse | 466 |
| C1- | probeset | (Fully | | 187498 | | | [Source: HGNC | 13 | Strand | |
| 1145a_s_at | | Exonic) | | | | | Symbol; Acc: 2202] | | | |
| OC3P.4984. | Expression | Sense | 11 | ENSG00000- | COL5A1 | 1289 | collagen, type V, alpha 1 | Chr | Forward | 467 |
| C1- | probeset | (Fully | | 130635 | | | [Source: HGNC | 9 | Strand | |
| 787a_s_at | | Exonic) | | | | | Symbol; Acc: 2209] | | | |
| OCHP.1005_ | Expression | Sense | 11 | ENSG00000- | COL5A1 | 1289 | collagen, type V, alpha 1 | Chr | Forward | 468 |
| s_at | probeset | (Fully | | 130635 | | | [Source: HGNC | 9 | Strand | |
| | | Exonic) | | | | | Symbol; Acc: 2209] | | | |
| OC3P.2713. | Expression | Sense | 11 | ENSG00000- | COL5A2 | 1290 | collagen, type V, alpha 2 | Chr | Reverse | 469 |
| C1_s_at | probeset | (Fully | | 204262 | | | [Source: HGNC | 2 | Strand | |
| | | Exonic) | | | | | Symbol; Acc: 2210] | | | |
| OC3P.13652. | Expression | Sense | 11 | ENSG00000- | COL8A1 | 1295 | collagen, type VIII, alpha 1 | Chr | Forward | 470 |
| C1_s_at | probeset | (Fully | | 144810 | | | [Source: HGNC | 3 | Strand | |
| | | Exonic) | | | | | Symbol; Acc: 2215] | | | |
| OC3P.10562. | Expression | Sense | 11 | ENSG00000- | COL8A1 | 1295 | collagen, type VIII, alpha 1 | Chr | Forward | 471 |
| C1_s_at | probeset | (Fully | | 144810 | | | [Source: HGNC | 3 | Strand | |
| | | Exonic) | | | | | Symbol; Acc: 2215] | | | |
| OC3SNG. | Expression | Sense | 11 | ENSG00000- | COL10A1 | 1300 | collagen, type X, alpha 1 | Chr | Reverse | 472 |
| 1834- | probeset | (Fully | | 123500 | | | [Source: HGNC | 6 | Strand | |
| 947a_s_at | | Exonic) | | | | | Symbol; Acc: 2185] | | | |
| OCRS.383_ | Expression | Sense | 11 | ENSG00000- | COL10A1 | 1300 | collagen, type X, alpha 1 | Chr | Reverse | 473 |
| s_at | probeset | (Fully | | 123500 | | | [Source: HGNC | 6 | Strand | |
| | | Exonic) | | | | | Symbol; Acc: 2185] | | | |
| OC3P.14073. | Expression | Sense | 11 | ENSG00000- | COL12A1 | 1303 | collagen, type XII, alpha 1 | Chr | Reverse | 474 |
| C1_s_at | probeset | (Fully | | 111799 | | | [Source: HGNC | 6 | Strand | |
| | | Exonic) | | | | | Symbol; Acc: 2188] | | | |
| OC3SNGnh. | Expression | Sense | 8 | ENSG00000- | COL12A1 | 1303 | collagen, type XII, alpha 1 | Chr | Reverse | 475 |
| 11427_ | probeset | (includes | | 111799 | | | [Source: HGNC | 6 | Strand | |
| x_at | | Intronic) | | | | | Symbol; Acc: 2188] | | | |
| OC3SNGnh. | Expression | Sense | 6 | ENSG00000- | COL12A1 | 1303 | collagen, type XII, alpha 1 | Chr | Reverse | 476 |
| 11427_ | probeset | (includes | | 111799 | | | [Source: HGNC | 6 | Strand | |
| at | | Intronic) | | | | | Symbol; Acc: 2188] | | | |
| OC3P.10157. | Expression | Sense | 11 | ENSG00000- | COL15A1 | 1306 | collagen, type XV, alpha 1 | Chr | Forward | 477 |
| C1_s_at | probeset | (Fully | | 204291 | | | [Source: HGNC | 9 | Strand | |
| | | Exonic) | | | | | Symbol; Acc: 2192] | | | |
| OCMX. | Expression | Anti- | 11 | ENSG00000- | KLF6 | 1316 | Kruppel-like factor 6 | Chr | Reverse | 478 |
| 4393. | probeset | Sense | | 067082 | | | [Source: HGNC | 10 | Strand | |
| C1_s_at | | | | | | | Symbol; Acc: 2235] | | | |
| OCMX. | Expression | Sense | 11 | ENSG00000- | VCAN | 1462 | versican | Chr | Forward | 479 |
| 15173. | probeset | (Fully | | 038427 | | | [Source: HGNC | 5 | Strand | |
| C1_s_at | | Exonic) | | | | | Symbol; Acc: 2464] | | | |
| OC3P.1200. | Expression | Sense | 11 | ENSG00000- | VCAN | 1462 | versican | Chr | Forward | 480 |
| C1_s_at | probeset | (Fully | | 038427 | | | [Source: HGNC | 5 | Strand | |
| | | Exonic) | | | | | Symbol; Acc: 2464] | | | |
| OCADNP. | Expression | Sense | 11 | ENSG00000- | CTGF | 1490 | connective tissue growth | Chr | Reverse | 481 |
| 9526_ | probeset | (Fully | | 118523 | | | factor [Source: HGNC | 6 | Strand | |
| s_at | | Exonic) | | | | | Symbol; Acc: 2500] | | | |
| OC3P.1178. | Expression | Sense | 9 | ENSG00000- | CTGF | 1490 | connective tissue growth | Chr | Reverse | 482 |
| C1_x_at | probeset | (Fully | | 118523 | | | factor [Source: HGNC | 6 | Strand | |
| | | Exonic) | | | | | Symbol; Acc: 2500] | | | |
| OC3P.1178. | Expression | Sense | 10 | ENSG00000- | CTGF | 1490 | connective tissue growth | Chr | Reverse | 483 |
| C1_at | probeset | (Fully | | 118523 | | | factor [Source: HGNC | 6 | Strand | |
| | | Exonic) | | | | | Symbol; Acc: 2500] | | | |
| OC3P.2822. | Expression | Sense | 11 | ENSG00000- | CYP27A1 | 1593 | cytochrome P450, family 27, | Chr | Forward | 484 |
| C1_s_at | probeset | (Fully | | 135929 | | | subfamily A, polypeptide 1 | 2 | Strand | |
| | | Exonic) | | | | | [Source: HGNC | | | |
| | | | | | | | Symbol; Acc: 2605] | | | |
| OC3P.694. | Expression | Sense | 8 | ENSG00000- | DCN | 1634 | decorin | Chr | Reverse | 485 |
| CB1- | probeset | (Fully | | 014665 | | | [Source: HGNC | 12 | Strand | |
| 490a_s_at | | Exonic) | | | | | Symbol; Acc: 2705] | | | |
| OC3SNG. | Expression | Sense | 11 | ENSG00000- | DCN | 1634 | decorin | Chr | Reverse | 486 |
| 461- | probeset | (Fully | | 011465 | | | [Source: HGNC | 12 | Strand | |
| 892a_s_at | | Exonic) | | | | | Symbol; Acc: 2705] | | | |
| OC3SNGnh. | Expression | Sense | 11 | ENSG00000- | DMD | 1756 | dystrophin | Chr | Reverse | 487 |
| 5811_at | probeset | (includes | | 198947 | | | [Source: HGNC | X | Strand | |
| | | Intronic) | | | | | Symbol; Acc: 2928] | | | |
| OCADNP. | Expression | Sense | 11 | ENSG00000- | DPYSL3 | 1809 | dihydropyrimidinase-like 3 | Chr | Reverse | 488 |
| 13759_ | probeset | (includes | | 113657 | | | [Source: HGNC | 5 | Strand | |
| s_at | | Intronic) | | | | | Symbol; Acc: 3015] | | | |
| OC3SNGnh. | Expression | Sense | 11 | ENSG00000- | DUSP4 | 1846 | dual specificity phosphate 4 | Chr | Reverse | 489 |
| 8739_s_at | probeset | (Fully | | 120875 | | | [Source: HGNC | 8 | Strand | |
| | | Exonic) | | | | | Symbol; Acc: 3070] | | | |
| OCADA. | Expression | Sense | 11 | ENSG00000- | EFNA5 | 1946 | ephrin-A5 | Chr | Reverse | 490 |
| 7893_s_at | probeset | (Fully | | 184349 | | | [Source: HGNC | 5 | Strand | |
| | | Exonic) | | | | | Symbol; Acc: 3225] | | | |
| OC3SNGnh. | Expression | Anti- | 11 | ENSG00000- | EFNA5 | 1946 | ephrin-A5 | Chr | Reverse | 491 |
| 9087_at | probeset | Sense | | 184349 | | | [Source: HGNC | 5 | Strand | |
| | | | | | | | Symbol; Acc: 3225] | | | |
| OC3P.1910. | Expression | Sense | 11 | ENSG00000- | EGR1 | 1958 | early growth response 1 | Chr | Forward | 492 |
| C1_s_at | probeset | (Fully | | 120738 | | | [Source: HGNC | 5 | Strand | |
| | | Exonic) | | | | | Symbol; Acc: 3238] | | | |
| OCADNP. | Expression | Anti- | 11 | ENSG00000- | EGR1 | 1958 | early growth response 1 | Chr | Forward | 493 |
| 2432_s_at | probeset | Sense | | 120738 | | | [Source: HGNC | 5 | Strand | |
| | | | | | | | Symbol; Acc: 3238] | | | |
| OC3SNGnh. | Expression | Anti- | 11 | ENSG00000- | EGR1 | 1958 | early growth response 1 | Chr | Forward | 494 |
| 19479_ | probeset | Sense | | 120738 | | | [Source: HGNC | 5 | Strand | |
| s_at | | | | | | | Symbol; Acc: 3238] | | | |
| OCMX.8. | Expression | Anti- | 11 | ENSG00000- | EGR1 | 1958 | early growth response 1 | Chr | Forward | 495 |
| C2_s_at | probeset | Sense | | 120738 | | | [Source: HGNC | 5 | Strand | |
| | | | | | | | Symbol; Acc: 3238] | | | |
| OC3SNGn. | Expression | Sense | 11 | ENSG00000- | EGR1 | 1958 | early growth response 1 | Chr | Forward | 496 |
| 469-921a_ | probeset | (Fully | | 120738 | | | [Source: HGNC | 5 | Strand | |
| s_at | | Exonic) | | | | | Symbol; Acc: 3238] | | | |
| OC3P.89. | Expression | Sense | 11 | ENSG00000- | ELN | 2006 | elastin | Chr | Forward | 497 |
| C6_s_at | probeset | (Fully | | 049540 | | | [Source: HGNC | 7 | Strand | |
| | | Exonic) | | | | | Symbol; Acc: 3327] | | | |
| OC3P.1292. | Expression | Sense | 11 | ENSG00000- | EMP1 | 2012 | epithelial membrane | Chr | Forward | 498 |
| C1_s_at | probeset | (Fully | | 134531 | | | protein 1 | 12 | Strand | |
| | | Exonic) | | | | | [Source: HGNC | | | |
| | | | | | | | Symbol; Acc: 3333] | | | |
| OC3SNG. | Expression | Sense | 11 | ENSG00000- | ETV1 | 2115 | ets variane 1 | Chr | Reverse | 499 |
| 2163- | probeset | (Fully | | 006468 | | | [Source: HGNC | 7 | Strand | |
| 2941a_s_at | | Exonic) | | | | | Symbol; Acc: 3490] | | | |
| OC3SNGnh. | Expression | Sense | 9 | ENSG00000- | ACSL4 | 2182 | acyl-CoA synthetase long- | Chr | Reverse | 500 |
| 1613_at | probeset | (includes | | 068366 | | | chain family member 4 | X | Strand | |
| | | Intronic) | | | | | [Source: HGNC | | | |
| | | | | | | | Symbol; Acc: 3571] | | | |
| OC3P.3499. | Expression | Sense | 11 | ENSG00000- | FAT1 | 2195 | FAT atypical cadherin 1 | Chr | Reverse | 501 |
| C1_s_at | probeset | (Fully | | 083857 | | | [Source: HGNC | 4 | Strand | |
| | | Exonic) | | | | | Symbol; Acc: 3595] | | | |
| OCHP. | Expression | Sense | 11 | ENSG00000- | FBLN2 | 2199 | fibulin 2 | Chr | Forward | 502 |
| 1078_s_at | probeset | (Fully | | 163520 | | | [Source: HGNC | 3 | Strand | |
| | | Exonic) | | | | | Symbol; Acc: 3601] | | | |
| OC3SNG. | Expression | Sense | 11 | ENSG00000- | FGFR1 | 2260 | fibroblast growth factor | Chr | Reverse | 503 |
| 6042- | probeset | (Fully | | 077782 | | | receptor 1 [Source: HGNC | 8 | Strand | |
| 23a_x_at | | Exonic) | | | | | Symbol; Acc: 3688] | | | |
| OCADNP. | Expression | Sense | 11 | ENSG00000- | FGFR1 | 2260 | fibroblast growth factor | Chr | Reverse | 504 |
| 8535_s_at | probeset | (Fully | | 077782 | | | receptor 1 [Source: HGNC | 8 | Strand | |
| | | Exonic) | | | | | Symbol; Acc: 3688] | | | |
| OC3SNGn. | Expression | Sense | 7 | ENSG00000- | FGFR1 | 2260 | fibroblast growth factor | Chr | Reverse | 505 |
| 6036- | probeset | (Fully | | 077782 | | | receptor 1 [Source: HGNC | 8 | Strand | |
| 20a_x_at | | Exonic) | | | | | Symbol; Acc: 3688] | | | |
| OCMXSNG. | Expression | Anti- | 11 | ENSG00000- | FN1 | 2335 | fibronectin 1 | Chr | Reverse | 506 |
| 5052_s_at | probeset | Sense | | 115414 | | | [Source: HGNC | 2 | Strand | |
| | | | | | | | Symbol; Acc: 3778] | | | |
| OCMX.493. | Expression | Sense | 11 | ENSG00000- | FN1 | 2335 | fibronectin 1 | Chr | Reverse | 507 |
| C1_s_at | probeset | (Fully | | 115414 | | | [Source: HGNC | 2 | Strand | |
| | | Exonic) | | | | | Symbol; Acc: 3778] | | | |
| OCADA. | Expression | Sense | 11 | ENSG00000- | FOS | 2353 | FBJ murine | Chr | Forward | 508 |
| 9921_s_at | probeset | (Fully | | 170345 | | | osteosarcoma viral | 14 | Strand | |
| | | Exonic) | | | | | oncogene homolog | | | |
| | | | | | | | [Source: HGNC | | | |
| | | | | | | | Symbol; Acc: 3796] | | | |
| OC3SNGnh. | Expression | Sense | 7 | ENSG00000- | FYN | 2534 | FYN oncogene related | Chr | Reverse | 509 |
| 12139_at | probeset | (Fully | | 010810 | | | to SRC, FGR, YES | 6 | Strand | |
| | | Exonic) | | | | | [Source: HGNC | | | |
| | | | | | | | Symbol; Acc: 4037] | | | |
| OCMX. | Expression | Anti- | 11 | ENSG00000- | GAS1 | 2619 | growth arrest-specific 1 | Chr | Reverse | 510 |
| 11023. | probeset | Sense | | 180447 | | | [Source: HGNC | 9 | Strand | |
| C1_s_at | | | | | | | Symbol; Acc: 4165] | | | |
| OCHP. | Expression | Sense | 11 | ENSG00000- | GJA1 | 2697 | gap junction protein, | Chr | Forward | 511 |
| 1836_s_at | probeset | (Fully | | 152661 | | | alpha 1, 43 kDa | 6 | Strand | |
| | | Exonic) | | | | | [Source: HGNC | | | |
| | | | | | | | Symbol; Acc: 4274] | | | |
| OCADA. | Expression | Sense | 11 | ENSG00000- | GSN | 2934 | gelsolin [Source: HGNC | Chr | Forward | 512 |
| 7782_s_at | probeset | (includes | | 148180 | | | Symbol; Acc: 4620] | 9 | Strand | |
| | | Intronic) | | | | | | | | |
| OC3SNGn. | Expression | Sense | 11 | ENSG00000- | HOXC6 | 3223 | homeobox C6 | Chr | Forward | 513 |
| 484- | probeset | (Fully | | 197757 | | | [Source: HGNC | 12 | Strand | |
| 1a_s_at | | Exonic) | | | | | Symbol; Acc: 5128] | | | |
| OC3P. | Expression | Sense | 11 | ENSG00000- | HOXC6 | 3223 | homeobox C6 | Chr | Forward | 514 |
| 10127. | probeset | (Fully | | 197757 | | | [Source: HGNC | 12 | Strand | |
| C1_s_at | | Exonic) | | | | | Symbol; Acc: 5128] | | | |
| OC3P.1878. | Expression | Sense | 11 | ENSG00000- | TNC | 3371 | tenascin C [Source: HGNC | Chr | Reverse | 515 |
| C1_s_at | probeset | (Fully | | 041982 | | | Symbol; Acc: 5318] | 9 | Strand | |
| | | Exonic) | | | | | | | | |
| OC3P. | Expression | Sense | 11 | ENSG00000- | IGF1 | 3479 | insulin-like growth factor 1 | Chr | Reverse | 516 |
| 12939. | probeset | (Fully | | 017427 | | | (somatomedin C) | 12 | Strand | |
| C1_s_at | | Exonic) | | | | | [Source: HGNC | | | |
| | | | | | | | Symbol; Acc: 5464] | | | |
| OCMX. | Expression | Anti- | 8 | ENSG00000- | IGF1 | 3479 | insulin-like growth factor 1 | Chr | Reverse | 517 |
| 11138. | probeset | Sense | | 017427 | | | (somatomedin C) | 12 | Strand | |
| C1_x_at | | | | | | | [Source: HGNC | | | |
| | | | | | | | Symbol; Acc: 5464] | | | |
| OCMX. | Expression | Anti- | 8 | ENSG00000- | IGF1 | 3479 | insulin-like growth factor 1 | Chr | Reverse | 518 |
| 11138. | probeset | Sense | | 017427 | | | (somatomedin C) | 12 | Strand | |
| C1_at | | | | | | | [Source: HGNC | | | |
| | | | | | | | Symbol; Acc: 5464] | | | |
| OC3P.460. | Expression | Anti- | 11 | ENSG00000- | IGF2 | 3481 | insulin-like growth factor 2 | Chr | Reverse | 519 |
| C1_s_at | probeset | Sense | | 167244 | | | (somatomedin A) | 11 | Strand | |
| | | | | | | | [Source: HGNC | | | |
| | | | | | | | Symbol; Acc: 5466] | | | |
| OC3SNG. | Expression | Sense | 11 | ENSG00000- | IGFBP4 | 3487 | insulin-like growth factor | Chr | Forward | 520 |
| 5134- | probeset | (Fully | | 141753 | | | binding protein 4 | 17 | Strand | |
| 22a_s_at | | Exonic) | | | | | [Source: HGNC | | | |
| | | | | | | | Symbol; Acc: 5473] | | | |
| OC3P. | Expression | Sense | 6 | ENSG00000- | IGFBP5 | 3488 | insulin-like growth factor | Chr | Reverse | 521 |
| 1987. | probeset | (Fully | | 115461 | | | binding protein 5 | 2 | Strand | |
| C1_x_at | | Exonic) | | | | | [Source: HGNC | | | |
| | | | | | | | Symbol; Acc: 5474] | | | |
| OC3P. | Expression | Sense | 9 | ENSG00000- | IGFBP5 | 3488 | insulin-like growth factor | Chr | Reverse | 522 |
| 1987. | probeset | (Fully | | 115461 | | | binding protein 5 | 2 | Strand | |
| CB1_x_at | | Exonic) | | | | | [Source: HGNC | | | |
| | | | | | | | Symbol; Acc: 5474] | | | |
| OC3SNG. | Expression | Sense | 9 | ENSG00000- | IGFBP5 | 3488 | insulin-like growth factor | Chr | Reverse | 523 |
| 2502- | probeset | (Fully | | 115461 | | | binding protein 5 | 2 | Strand | |
| 79a_s_at | | Exonic) | | | | | [Source: HGNC | | | |
| | | | | | | | Symbol; Acc: 5474] | | | |
| OCADNP. | Expression | Sense | 11 | ENSG00000- | IGFBP5 | 3488 | insulin-like growth factor | Chr | Reverse | 524 |
| 830_s_at | probeset | (Fully | | 115461 | | | binding protein 5 | 2 | Strand | |
| | | Exonic) | | | | | [Source: HGNC | | | |
| | | | | | | | Symbol; Acc: 5474] | | | |
| OC3SNGnh. | Expression | Anti- | 11 | ENSG00000- | IGFBP5 | 3488 | insulin-like growth factor | Chr | Reverse | 525 |
| 6980_s_at | probeset | Sense | | 115461 | | | binding protein 5 | 2 | Strand | |
| | | | | | | | [Source: HGNC | | | |
| | | | | | | | Symbol; Acc: 5474] | | | |
| OCHP. | Expression | Sense | 11 | ENSG00000- | IGFBP6 | 3489 | insulin-like growth factor | Chr | Forward | 526 |
| 1301_s_at | probeset | (Fully | | 167779 | | | binding protein 6 | 12 | Strand | |
| | | Exonic) | | | | | [Source: HGNC | | | |
| | | | | | | | Symbol; Acc: 5475] | | | |
| OCADNP. | Expression | Sense | 6 | ENSG00000- | IGFBP7 | 3490 | insulin-like growth factor | Chr | Reverse | 527 |
| 3131_x_at | probeset | (includes | | 163453 | | | binding protein 7 | 4 | Strand | |
| | | Intronic) | | | | | [Source: HGNC | | | |
| | | | | | | | Symbol; Acc: 5476] | | | |
| OC3P. | Expression | Sense | 10 | ENSG00000- | IL1R1 | 3554 | interleukin 1 | Chr | Forward | 528 |
| 1843. | probeset | (Fully | | 115594 | | | receptor, type I | 2 | Strand | |
| C1_s_at | | Exonic) | | | | | [Source: HGNC | | | |
| | | | | | | | Symbol; Acc: 5993] | | | |
| OC3P. | Expression | Sense | 11 | ENSG00000- | IRS1 | 3667 | insulin | Chr | Reverse | 529 |
| 5893. | probeset | (Fully | | 169047 | | | receptor substrate 1 | 2 | Strand | |
| C1_s_at | | Exonic) | | | | | [Source: HGNC | | | |
| | | | | | | | Symbol; Acc: 6125] | | | |
| OCHP. | Expression | Sense | 11 | ENSG00000- | KIT | 3815 | v-kit Hardy-Zuckerman 4 | Chr | Forward | 530 |
| 1881_s_at | probeset | (Fully | | 157404 | | | feline sarcoma viral | 4 | Strand | |
| | | Exonic) | | | | | oncogene homolog | | | |
| | | | | | | | [Source: HGNC | | | |
| | | | | | | | Symbol; Acc: 6342] | | | |
| OC3SNGnh. | Expression | Sense | 11 | ENSG00000- | ABLIM1 | 3983 | actin binding LIM | Chr | Reverse | 531 |
| 985_s_at | probeset | (Fully | | 099204 | | | protein 1 | 10 | Strand | |
| | | Exonic) | | | | | [Source: HGNC | | | |
| | | | | | | | Symbol; Acc: 78] | | | |
| OCHP. | Expression | Sense | 11 | ENSG00000- | LOXL1 | 4016 | lysl oxidase-like 1 | Chr | Forward | 532 |
| 1306_s_at | probeset | (Fully | | 129038 | | | [Source: HGNC | 15 | Strand | |
| | | Exonic) | | | | | Symbol; Acc: 6665] | | | |
| OCMXSNG. | Expression | Anti- | 7 | ENSG00000- | LOXL1 | 4016 | lysl oxidase-like 1 | Chr | Forward | 533 |
| 3759_x_at | probeset | Sense | | 129038 | | | [Source: HGNC | 15 | Strand | |
| | | | | | | | Symbol; Acc: 6665] | | | |
| OC3P. | Expression | Sense | 11 | ENSG00000- | LTBP2 | 4053 | latent transforming growth | Chr | Reverse | 534 |
| 5700. | probeset | (Fully | | 119681 | | | factor beta binding protein | 14 | Strand | |
| C1_s_at | | Exonic) | | | | | 2 [Source: HGNC | | | |
| | | | | | | | Symbol; Acc: 6715] | | | |
| OCHP. | Expression | Sense | 11 | ENSG00000- | LUM | 4060 | lumican | Chr | Reverse | 535 |
| 1534_s_at | probeset | (Fully | | 139329 | | | [Source: HGNC | 12 | Strand | |
| | | Exonic) | | | | | Symbol; Acc: 6724] | | | |
| OC3P. | Expression | Sense | 11 | ENSG00000- | MARCKS | 4082 | myristoylated alanine-rich | Chr | Forward | 536 |
| 9248. | probeset | (Fully | | 155130 | | | protein kinase C substrate | 6 | Strand | |
| C1_s_at | | Exonic) | | | | | [Source: HGNC | | | |
| | | | | | | | Symbol; Acc: 6759] | | | |
| OC3P. | Expression | Sense | 11 | ENSG00000- | MARCKS | 4082 | myristoylated alanine-rich | Chr | Forward | 537 |
| 10485. | probeset | (Fully | | 155130 | | | protein kinase C substrate | 6 | Strand | |
| C1_s_at | | Exonic) | | | | | [Source: HGNC | | | |
| | | | | | | | Symbol; Acc: 6759] | | | |
| OCADA. | Expression | Sense | 11 | ENSG00000- | MAP3K1 | 4214 | mitogen-activated protein | Chr | Forward | 538 |
| 6829_s_at | probeset | (includes | | 095015 | | | kinase kinase kinase 1, E3 | 5 | Strand | |
| | | Intronic) | | | | | ubiquitin protein ligase | | | |
| | | | | | | | [Source: HGNC | | | |
| | | | | | | | Symbol; Acc: 6848] | Chr | Reverse | 539 |
| OC3SNGn. | Expression | Sense | 9 | ENSG00000- | MGP | 4256 | matrix Gla protein | 12 | Strand | |
| 8705- | probeset | (Fully | | 111341 | | | [Source: HGNC | | | |
| 760a_x_at | | Exonic) | | | | | Symbol; Acc: 7060] | | | |
| OCADNP. | Expression | Sense | 11 | ENSG00000- | MMP2 | 4313 | matrix metallopeptidase 2 | Chr | Forward | 540 |
| 7251_s_at | probeset | (Fully | | 087245 | | | (gelatinase A 72 kDa | 16 | Strand | |
| | | Exonic) | | | | | gelatinase, 72 kDa type IV | | | |
| | | | | | | | collagenase [Source: HGNC | | | |
| | | | | | | | Symbol; Acc: 7166] | | | |
| OC3SNGn. | Expression | Sense | 11 | ENSG00000- | MMP11 | 4320 | matrix metallopeptidase 11 | Chr | Forward | 541 |
| 2375- | probeset | (Fully | | 099953 | | | (stromelysin 3) | 22 | Strand | |
| 26a_s_at | | Exonic) | | | | | [Source: HGNC | | | |
| | | | | | | | Symbol; Acc: 7157] | | | |
| OC3P. | Expression | Sense | 11 | ENSG00000- | MMP11 | 4320 | matrix metallopeptidase 11 | Chr | Forward | 542 |
| 3764. | probeset | (Fully | | 099953 | | | (stromelysin 3) | 22 | Strand | |
| C1_s_at | | Exonic) | | | | | [Source: HGNC | | | |
| | | | | | | | Symbol; Acc: 7157] | | | |
| OC3P. | Expression | Sense | 9 | ENSG00000- | MMP14 | 4323 | matrix metallopeptidase 14 | Chr | Forward | 543 |
| 4123. | probeset | (Fully | | 157227 | | | (membrane-inserted) | 14 | Strand | |
| C1_x_at | | Exonic) | | | | | [Source: HGNC | | | |
| | | | | | | | Symbol; Acc: 7160] | | | |
| OCADA. | Expression | Sense | 11 | ENSG00000- | MSN | 4478 | moesin | Chr | Forward | 544 |
| 6468_s_at | probeset | (includes | | 147065 | | | [Source: HGNC | X | Strand | |
| | | Intronic) | | | | | Symbol; Acc: 7373] | | | |
| OCHPRC. | Expression | Sense | 11 | ENSG00000- | MSX1 | 4487 | msh homeobox 1 | Chr | Forward | 545 |
| 15_s_at | probeset | (Fully | | 163132 | | | [Source: HGNC | 4 | Strand | |
| | | Exonic) | | | | | Symbol; Acc: 7391] | | | |
| OCADNP. | Expression | Sense | 6 | ENSG00000- | MUC6 | 4588 | mucin 6, oligomeric mucus/ | Chr | Reverse | 546 |
| 1685_s_at | probeset | (Fully | | 184956 | | | gel-forming | 11 | Strand | |
| | | Exonic) | | | | | [Source: HGNC | | | |
| | | | | | | | Symbol; Acc: 7517] | | | |
| OC3P. | Expression | Sense | 11 | ENSG00000- | GADD45B | 4616 | growth arrest and DNA- | Chr | Forward | 547 |
| 4001. | probeset | (Fully | | 099860 | | | damage-inducible, beta | 19 | Strand | |
| C1_s_at | | Exonic) | | | | | [Source: HGNC | | | |
| | | | | | | | Symbol; Acc: 4096] | | | |
| OC3P. | Expression | Sense | 11 | ENSG00000- | NDN | 4692 | necdin, melanoma antigen | Chr | Reverse | 548 |
| 3034. | probeset | (Fully | | 182636 | | | (MAGE) family member | 15 | Strand | |
| C1_s_at | | Exonic) | | | | | [Source: HGNC | | | |
| | | | | | | | Symbol; Acc: 7675] | | | |
| OC3P. | Expression | Sense | 11 | ENSG00000- | NFATC1 | 4772 | nuclear factor of activated | Chr | Forward | 549 |
| 12852. | probeset | (Fully | | 131196 | | | T-cells, cytoplasmic, | 18 | Strand | |
| C1_s_at | | Exonic) | | | | | calcineurin-dependent 1 | | | |
| | | | | | | | [Source: HGNC | | | |
| | | | | | | | Symbol; Acc: 7775] | | | |
| OC3P. | Expression | Sense | 11 | ENSG00000- | NPAS2 | 4862 | neuronal PAS domain | Chr | Forward | 550 |
| 6842. | probeset | (Fully | | 170485 | | | protein 2 | 2 | Strand | |
| C1_s_at | | Exonic) | | | | | [Source: HGNC | | | |
| | | | | | | | Symbol; Acc: 7895] | | | |
| OC3SNG. | Expression | Sense | 11 | ENSG00000- | DDR2 | 4921 | discoidin domain receptor | Chr | Forward | 551 |
| 1306- | probeset | (Fully | | 162733 | | | tyrosine kinase 2 | 1 | Strand | |
| 60a_s_at | | Exonic) | | | | | [Source: HGNC | | | |
| | | | | | | | Symbol; Acc: 2731] | | | |
| OCHP. | Expression | Sense | 11 | ENSG00000- | PDGFRA | 5156 | platelet-derived growth | Chr | Forward | 552 |
| 769_s_at | probeset | (Fully | | 134853 | | | factor receptor, | 4 | Strand | |
| | | Exonic) | | | | | alpha polypeptide | | | |
| | | | | | | | [Source: HGNC | | | |
| | | | | | | | Symbol; Acc: 8803] | | | |
| OC3P. | Expression | Sense | 11 | ENSG00000- | PFKFB3 | 5209 | 6-phosphofructo-2- | Chr | Forward | 553 |
| 4849. | probeset | (Fully | | 170525 | | | kinase/fructose-2,6- | 10 | Strand | |
| C1_s_at | | Exonic) | | | | | biphosphatase 2 [Source: | | | |
| | | | | | | | HGNC Symbol; Acc: 8874] | | | |
| OCHP. | Expression | Sense | 11 | ENSG00000- | PLAU | 5328 | plasminogen activator, | Chr | Forward | 554 |
| 739_s_at | probeset | (Fully | | 122861 | | | urokinase | 10 | Strand | |
| | | Exonic) | | | | | [Source: HGNC | | | |
| | | | | | | | Symbol; Acc: 9052] | | | |
| OC3P. | Expression | Sense | 11 | ENSG00000- | RASGRF2 | 5924 | Ras protein-specific guanine | Chr | Forward | 555 |
| 12892. | probeset | (Fully | | 113319 | | | nucleotide-releasing factor 2 | 5 | Strand | |
| C1_s_at | | Exonic) | | | | | [Source: HGNC | | | |
| | | | | | | | Symbol; Acc: 9876] | | | |
| OCRS. | Expression | Sense | 11 | ENSG00000- | RFXAP | 5994 | regulatory factor X- | Chr | Forward | 556 |
| 1674_s_at | probeset | (Fully | | 133111 | | | associated protein | 13 | Strand | |
| | | Exonic) | | | | | [Source: HGNC | | | |
| | | | | | | | Symbol; Acc: 9988] | | | |
| OC3P. | Expression | Sense | 11 | ENSG00000- | RGS2 | 5997 | regulatory of G-protein | Chr | Forward | 557 |
| 3100. | probeset | (Fully | | 116741 | | | signalling 2, 24 kDa | 1 | Strand | |
| C1_s_at | | Exonic) | | | | | [Source: HGNC | | | |
| | | | | | | | Symbol; Acc: 9988] | | | |
| OC3P. | Expression | Sense | 11 | ENSG00000- | ROBO1 | 6091 | roundabout, axon guidance | Chr | Reverse | 558 |
| 13061. | probeset | (Fully | | 169855 | | | receptor, homolog 1 | 3 | Strand | |
| C1_s_at | | Exonic) | | | | | (Drosophila) | | | |
| | | | | | | | [Source: HGNC | | | |
| | | | | | | | Symbol; Acc: 10249] | | | |
| OS3SNGnh. | Expression | Sense | 7 | ENSG00000- | RORA | 6095 | RAR-related orphan | Chr | Reverse | 559 |
| 14507_x_at | probeset | (includes | | 069667 | | | receptor A | 15 | Strand | |
| | | Intronic) | | | | | [Source: HGNC | | | |
| | | | | | | | Symbol; Acc: 9988] | | | |
| OS3SNGnh. | Expression | Sense | 7 | ENSG00000- | RORA | 6095 | RAR-related orphan | Chr | Reverse | 560 |
| 14507_x_at | probeset | (includes | | 069667 | | | receptor A | 15 | Strand | |
| | | Intronic) | | | | | [Source: HGNC | | | |
| | | | | | | | Symbol; Acc: 9988] | | | |
| OS3SNGnh. | Expression | Sense | 11 | ENSG00000- | RORA | 6095 | RAR-related orphan | Chr | Reverse | 561 |
| 5170_x_at | probeset | (includes | | 069667 | | | receptor A | 15 | Strand | |
| | | Intronic) | | | | | [Source: HGNC | | | |
| | | | | | | | Symbol; Acc: 10258] | | | |
| OC3P. | Expression | Sense | 11 | ENSG00000- | SDC2 | 6383 | syndecan 2 | Chr | Forward | 562 |
| 8373. | probeset | (Fully | | 169439 | | | [Source: HGNC | 8 | Strand | |
| C1_s_at | | Exonic) | | | | | Symbol; Acc: 10659] | | | |
| OC3P. | Expression | Sense | 11 | ENSG00000- | SFRP2 | 6423 | secreted frizzled-related | Chr | Reverse | 563 |
| 13621. | probeset | (Fully | | 145423 | | | protein 2 | 4 | Strand | |
| C1_s_at | | Exonic) | | | | | [Source: HGNC | | | |
| | | | | | | | Symbol; Acc: 10777] | | | |
| OC3P. | Expression | Sense | 11 | ENSG00000- | SGCB | 6443 | sarcogylcan. beta (43 kDa | Chr | Reverse | 564 |
| 7062. | probeset | (Fully | | 163069 | | | dystrophin-associated | 4 | Strand | |
| C1_s_at | | Exonic) | | | | | glycoprotein) | | | |
| | | | | | | | [Source: HGNC | | | |
| | | | | | | | Symbol; Acc: 10806] | | | |
| OC3SNG. | Expression | Sense | 11 | ENSG00000- | SMARCA1 | 6594 | SWI/SNF related, matrix | Chr | Reverse | 565 |
| 1640. | probeset | (Fully | | 102038 | | | associated, actin dependent | X | Strand | |
| 14a_s_at | | Exonic) | | | | | regulator of chromatin, | | | |
| | | | | | | | subfamily a, member 1 | | | |
| | | | | | | | [Source: HGNC | | | |
| | | | | | | | Symbol; Acc: 11097] | | | |
| OCRS2. | Expression | Sense | 11 | ENSG00000- | TAGLN | 6876 | transgelin | Chr | Forward | 566 |
| 11009_ | probeset | (Fully | | 149591 | | | [Source: HGNC | 11 | Strand | |
| x_at | | Exonic) | | | | | Symbol; Acc: | | | |
| | | | | | | | 11553] | | | |
| OC3P. | Expression | Sense | 8 | ENSG00000- | NR2F1 | 7025 | nuclear receptor subfamily | Chr | Forward | 567 |
| 5101. | probeset | (Fully | | 175745 | | | 2, group F, member 1 | 5 | Strand | |
| C1_s_at | | Exonic) | | | | | [Source: HGNC | | | |
| | | | | | | | Symbol; Acc: 7975] | | | |
| OS3SNGnh. | Expression | Sense | 11 | ENSG00000- | TGFB2 | 7042 | transforming growth factor, | Chr | Forward | 568 |
| 3734_s_at | probeset | (Fully | | 092969 | | | beta 2 | 1 | Strand | |
| | | Exonic) | | | | | [Source: HGNC | | | |
| | | | | | | | Symbol; Acc: 11768] | | | |
| OC3P. | Expression | Sense | 11 | ENSG00000- | TGFB3 | 7043 | transforming growth factor, | Chr | Reverse | 569 |
| 10233. | probeset | (Fully | | 119699 | | | beta 3 | 14 | Strand | |
| C1_s_at | | Exonic) | | | | | [Source: HGNC | | | |
| | | | | | | | Symbol; Acc: 11769] | | | |
| OC3P. | Expression | Sense | 9 | ENSG00000- | THBS1 | 7057 | thrombospondin 1 | Chr | Forward | |
| 11604. | probeset | (Fully | | 137801 | | | [Source: HGNC | 15 | Strand | |
| C1_s_at | | Exonic) | | | | | Symbol; | | | |
| | | | | | | | Acc: 11785] | | | |
| OC3P. | Expression | Sense | 11 | ENSG00000- | THY1 | 7070 | Thy-1 cell | Chr | Forward | 571 |
| 2790. | probeset | (Fully | | 154096 | | | surface antigen | 11 | Strand | |
| C1_s_at | | Exonic) | | | | | [Source: HGNC | | | |
| | | | | | | | Symbol; Acc: 11801] | | | |
| OC3P. | Expression | Sense | 11 | ENSG00000- | TIMP2 | 7077 | TIMP metallopeptidase | Chr | Forward | 572 |
| 543. | probeset | (Fully | | 035862 | | | inhibitor 2 | 17 | Strand | |
| CB1- | | Exonic) | | | | | [Source: HGNC | | | |
| 699a_s_at | | | | | | | Symbol; Acc: 11821] | | | |
| OC3SNGnh. | Expression | Sense | 11 | ENSG00000- | TIMP2 | 7077 | TIMP metallopeptidase | Chr | Forward | 573 |
| 19238_s_at | probeset | (Fully | | 035862 | | | inhibitor 2 | 17 | Strand | |
| | | Exonic) | | | | | [Source: HGNC | | | |
| | | | | | | | Symbol; Acc: 11821] | | | |
| OC3P. | Expression | Sense | 11 | ENSG00000- | TIMP3 | 7078 | TIMP metallopeptidase | Chr | Forward | 574 |
| 10470. | probeset | (Fully | | 100234 | | | inhibitor 3 | 22 | Strand | |
| C1_s_at | | Exonic) | | | | | [Source: HGNC | | | |
| | | | | | | | Symbol; Acc: 11822] | | | |
| OCADA. | Expression | Sense | 11 | ENSG00000- | TPM1 | 7168 | tropomyosin 1 | Chr | Forward | 575 |
| 8344_s_at | probeset | (includes | | 140416 | | | (alpha) | 15 | Strand | |
| | | Intronic) | | | | | [Source: HGNC | | | |
| | | | | | | | Symbol; Acc: 12010] | | | |
| OC3SNGnh. | Expression | Sense | 7 | ENSG00000- | TPM1 | 7168 | tropomyosin 1 | Chr | Forward | 576 |
| 487_at | probeset | (Fully | | 140416 | | | (alpha) | 15 | Strand | |
| | | Exonic) | | | | | [Source: HGNC | | | |
| | | | | | | | Symbol; Acc: 12010] | | | |
| OC3SNGnh. | Expression | Sense | 10 | ENSG00000- | TPM1 | 7168 | tropomyosin 1 | Chr | Forward | 577 |
| 5090_at | probeset | (Fully | | 140416 | | | (alpha) | 15 | Strand | |
| | | Exonic) | | | | | [Source: HGNC | | | |
| | | | | | | | Symbol; Acc: 12010] | | | |
| OCHP. | Expression | Sense | 11 | ENSG00000- | TRAF5 | 7188 | TNF receptor-associated | Chr | Forward | 578 |
| 643_s_at | probeset | (Fully | | 082512 | | | factor 5 | 1 | Strand | |
| | | Exonic) | | | | | [Source: HGNC | | | |
| | | | | | | | Symbol; Acc: 12035] | | | |
| OCADNP. | Expression | Anti- | 11 | ENSG00000- | TRIO | 7204 | trio Rho guanine nucleotide | Chr | Forward | 579 |
| 14769_s_at | probeset | Sense | | 038382 | | | exchange factor | 5 | Strand | |
| | | | | | | | [Source: HGNC | | | |
| | | | | | | | Symbol; Acc: 12303] | | | |
| OCRS2. | Expression | Sense | 11 | ENSG00000- | TWIST1 | 7291 | twist family bHLH | Chr | Reverse | 580 |
| 11542_s_at | probeset | (Fully | | 122691 | | | transcription factor 1 | 7 | Strand | |
| | | Exonic) | | | | | [Source: HGNC | | | |
| | | | | | | | Symbol; Acc: 12428] | | | |
| OC3SNGn. | Expression | Sense | 11 | ENSG00000- | TWIST1 | 7291 | twist family bHLH | Chr | Reverse | 581 |
| 2801- | probeset | (Fully | | 122691 | | | transcription factor 1 | 7 | Strand | |
| 166a_s_at | | Exonic) | | | | | [Source: HGNC | | | |
| | | | | | | | Symbol; Acc: 12428] | | | |
| OCMXSNG. | Expression | Anti- | 10 | ENSG00000- | TWIST1 | 7291 | twist family bHLH | Chr | Reverse | 582 |
| 2027_x_at | probeset | Sense | | 122691 | | | transcription factor 1 | 7 | Strand | |
| | | | | | | | [Source: HGNC | | | |
| | | | | | | | Symbol; Acc: 12428] | | | |
| OCMXSNG. | Expression | Anti- | 9 | ENSG00000- | TWIST1 | 7291 | twist family bHLH | Chr | Reverse | 583 |
| 2027_at | probeset | Sense | | 122691 | | | transcription factor 1 | 7 | Strand | |
| | | | | | | | [Source: HGNC | | | |
| | | | | | | | Symbol; Acc: 12428] | | | |
| OC3P. | Expression | Sense | 11 | ENSG00000- | TYRO3 | 7301 | TYRO3 protein | Chr | Forward | 584 |
| 5849. | probeset | (Fully | | 092445 | | | tyrosine kinase | 15 | Strand | |
| C1_s_at | | Exonic) | | | | | [Source: HGNC | | | |
| | | | | | | | Symbol; Acc: 12446] | | | |
| OC3P. | Expression | Sense | 11 | ENSG00000- | COL14A1 | 7373 | collagen, type XIV, | Chr | Forward | 585 |
| 7845. | probeset | (Fully | | 187955 | | | alpha 1 | 8 | Strand | |
| C1_s_at | | Exonic) | | | | | [Source: HGNC | | | |
| | | | | | | | Symbol; Acc: 12191 | | | |
| OC3SNGn. | Expression | Sense | 11 | ENSG00000- | COL14A1 | 7373 | collagen, type XIV, | Chr | Forward | 586 |
| 6594- | probeset | (Fully | | 187955 | | | alpha 1 | 8 | Strand | |
| 7a_s_at | | Exonic) | | | | | [Source: HGNC | | | |
| | | | | | | | Symbol; Acc: 12191 | | | |
| OCADA. | Expression | Anti- | 11 | ENSG00000- | WNT5A | 7474 | wingless-type | Chr | Reverse | 587 |
| 7569_s_at | probeset | Sense | | 114251 | | | MMTV integration | 3 | Strand | |
| | | | | | | | site family, member 5A | | | |
| | | | | | | | [Source: HGNC | | | |
| | | | | | | | Symbol; Acc: 12784] | | | |
| OC3SNG. | Expression | Sense | 11 | ENSG00000- | WNT7A | 7476 | wingless-type | Chr | Reverse | 588 |
| 1705- | probeset | (Fully | | 154764 | | | MMTV integration | 3 | Strand | |
| 33a_s_at | | Exonic) | | | | | site family, member 7A | | | |
| | | | | | | | [Source: HGNC | | | |
| | | | | | | | Symbol; Acc: 12786] | | | |
| OCMXSNG. | Expression | Sense | 11 | ENSG00000- | XIST | 7503 | X inactive specific transcript | Chr | Reverse | 589 |
| 4570_s_at | probeset | (Fully | | 229807 | | | (non-protein coding) | X | Strand | |
| | | Exonic) | | | | | [Source: HGNC | | | |
| | | | | | | | Symbol; Acc: 12810] | | | |
| OC3P. | Expression | Sense | 11 | ENSG00000- | XIST | 7503 | X inactive specific transcript | Chr | Reverse | 590 |
| 4145. | probeset | (Fully | | 229807 | | | (non-protein coding) | X | Strand | |
| C1_s_at | | Exonic) | | | | | [Source: HGNC | | | |
| | | | | | | | Symbol; Acc: 12810] | | | |
| OCMXSNG. | Expression | Sense | 11 | ENSG00000- | XIST | 7503 | X inactive specific transcript | Chr | Reverse | 591 |
| 427_s_at | probeset | (Fully | | 229807 | | | (non-protein coding) | X | Strand | |
| | | Exonic) | | | | | [Source: HGNC | | | |
| | | | | | | | Symbol; Acc: 12810] | | | |
| OCMX. | Expression | Sense | 11 | ENSG00000- | TSIX | 9383 | TSIX transcript. XIST | Chr | Forward | 592 |
| 1166. | probeset | (Fully | | 270641 | | | antisense RNA | X | Strand | |
| C2_at | | Exonic) | | | | | [Source: HGNC | | | |
| | | | | | | | Symbol; Acc: 12377] | | | |
| OCADNP. | Expression | Sense | 11 | ENSG00000- | XIST | 7503 | X inactive specific transcript | Chr | Reverse | 593 |
| 5655_s_at | probeset | (Fully | | 229807 | | | (non-protein coding) | X | Strand | |
| | | Exonic) | | | | | [Source: HGNC | | | |
| | | | | | | | Symbol; Acc: 12810] | | | |
| OCMXSNG. | Expression | Sense | 11 | ENSG00000- | XIST | 7503 | X inactive specific transcript | Chr | Reverse | 594 |
| 4891_s_at | probeset | (Fully | | 229807 | | | (non-protein coding) | X | Strand | |
| | | Exonic) | | | | | [Source: HGNC | | | |
| | | | | | | | Symbol; Acc: 12810] | | | |
| OC3SNGn. | Expression | Sense | 11 | ENSG00000- | ZFP36 | 7538 | ZFP36 ring | Chr | Forward | 595 |
| 1637- | probeset | (Fully | | 128016 | | | finger protein | 19 | Strand | |
| 35a_s_at | | Exonic) | | | | | [Source: HGNC | | | |
| | | | | | | | Symbol; Acc: 12862] | | | |
| OC3P. | Expression | Sense | 11 | ENSG00000- | ZNF175 | 7728 | zinc finger | Chr | Forward | 596 |
| 4040. | probeset | (Fully | | 105497 | | | protein 175 | 19 | Strand | |
| C1_s_at | | Exonic) | | | | | [Source: HGNC | | | |
| | | | | | | | Symbol; Acc: 12964] | | | |
| OC3P. | Expression | Sense | 11 | ENSG00000- | BTG2 | 7832 | BTG family, | Chr | Forward | 597 |
| 305.C1_at | probeset | (Fully | | 159388 | | | member 2 | 1 | Strand | |
| | | Exonic) | | | | | [Source: HGNC | | | |
| | | | | | | | Symbol; Acc: 1131] | | | |
| OCMX. | Expression | Anti- | 11 | ENSG00000- | PPFIBP1 | 8496 | PTPRF interacting protein, | Chr | Forward | 598 |
| 12937. | probeset | Sense | | 110841 | | | binding protein | 12 | Strand | |
| C1_s_at | | | | | | | 1 (liprin beta 1) | | | |
| | | | | | | | [Source: HGNC | | | |
| | | | | | | | Symbol; Acc: 9249] | | | |
| OCMX. | Expression | Sense | 11 | ENSG00000- | ENC1 | 8507 | ectodermal-neural cortex 1 | Chr | Reverse | 599 |
| 2061. | probeset | (Fully | | 171617 | | | (with BTB domain) | 5 | Strand | |
| C1_s_at | | Exonic) | | | | | [Source: HGNC | | | |
| | | | | | | | Symbol; Acc: 3345] | | | |
| OC3P. | Expression | Sense | 11 | ENSG00000- | GAS7 | 8522 | growth arrest- | Chr | Reverse | 600 |
| 8087. | probeset | (Fully | | 007237 | | | specific 7 | 17 | Strand | |
| C1_s_at | | Exonic) | | | | | [Source: HGNC | | | |
| | | | | | | | Symbol; Acc: 4169] | | | |
| OC3P. | Expression | Sense | 11 | ENSG00000- | IRS2 | 8660 | insulin receptor | Chr | Reverse | 601 |
| 13634. | probeset | (Fully | | 185950 | | | substrate 2 | 13 | Strand | |
| C1_s_at | | Exonic) | | | | | [Source: HGNC | | | |
| | | | | | | | Symbol; Acc: 6126] | | | |
| OCADNP. | Expression | Anti- | 6 | ENSG00000- | NRP2 | 8828 | neuropilin 2 | Chr | Forward | 602 |
| 2524_s_at | probeset | Sense | | 118257 | | | [Source: HGNC | 2 | Strand | |
| | | | | | | | Symbol; Acc: 8005] | | | |
| OC3P. | Expression | Sense | 11 | ENSG00000- | NRP1 | 8829 | neuropilin 1 | Chr | Reverse | 603 |
| 8445. | probeset | (Fully | | 099250 | | | [Source: HGNC | 10 | Strand | |
| C1_s_at | | Exonic) | | | | | Symbol; Acc: 8004] | | | |
| OCADA. | Expression | Sense | 11 | ENSG00000- | P4HA2 | 8974/// | prolyl 4-hydroxylase, alpha | Chr | Reverse | 604 |
| 8635_s_at | probeset | (includes | | 072682 | | 101927705 | polypeptide II | 5 | Strand | |
| | | Intronic) | | | | | [Source: HGNC | | | |
| | | | | | | | Symbol; Acc: 8547] | | | |
| OCADNP. | Expression | Sense | 11 | ENSG00000- | ASH2L | 9070 | ash2 (absent, small, or | Chr | Forward | 605 |
| 2893_s_at | probeset | (includes | | 129691 | | | homeotic)-like (Drosophila) | 8 | Strand | |
| | | Intronic) | | | | | [Source: HGNC | | | |
| | | | | | | | Symbol; Acc: 744] | | | |
| OC3P. | Expression | Sense | 11 | ENSG00000- | RGN | 9104 | regucalcin | Chr | Forward | 606 |
| 10790. | probeset | (Fully | | 130988 | | | [Source: HGNC | X | Strand | |
| C1_s_at | | Exonic) | | | | | Symbol; Acc: 9989] | | | |
| OC3P. | Expression | Sense | 11 | ENSG00000- | ECEL1 | 9427 | endothelin converting | Chr | Reverse | 607 |
| 12529. | probeset | (Fully | | 171551 | | | enzyme-like 1 | 2 | Strand | |
| C1_s_at | | Exonic) | | | | | [Source: HGNC | | | |
| | | | | | | | Symbol; Acc: 3147] | | | |
| OCHPRC. | Expression | Sense | 11 | ENSG00000- | ADAMTS2 | 9509 | ADAM metallopeptidase | Chr | Reverse | 608 |
| 106_s_at | probeset | (Fully | | 087116 | | | with thrombospondin | 5 | Strand | |
| | | Exonic) | | | | | type 1 motif, | | | |
| | | | | | | | 2 [Source: HGNC | | | |
| | | | | | | | Symbol; Acc: 218] | | | |
| OCHP. | Expression | Sense | 11 | ENSG00000- | CXCL14 | 9547 | chemokine (C-X-C motif) | Chr | Reverse | 609 |
| 1072_s_at | probeset | (Fully | | 145824 | | | ligand 14 | 5 | Strand | |
| | | Exonic) | | | | | [Source: HGNC | | | |
| | | | | | | | Symbol; Acc: 10640] | | | |
| OC3SNGnh. | Expression | Sense | 11 | ENSG00000- | DOCK4 | 9732 | dedicator of | Chr | Reverse | 610 |
| 868_x_at | probeset | (includes | | 128512 | | | cytokinesis 4 | 7 | Strand | |
| | | Intronic) | | | | | [Source: HGNC | | | |
| | | | | | | | Symbol; Acc: 19192] | | | |
| OC3P. | Expression | Sense | 11 | ENSG00000- | HEPH | 9843 | hephaestin | Chr | Forward | 611 |
| 6175. | probeset | (Fully | | 089472 | | | [Source: HGNC | X | Strand | |
| C1_s_at | | Exonic) | | | | | Symbol; Acc: 4866] | | | |
| OC3SNGn. | Expression | Sense | 11 | ENSG00000- | AKT3 | 10000 | v-akt murine thymoma viral | Chr | Reverse | 612 |
| 1784- | probeset | (Fully | | 117020 | | | oncogene homolog 3 | 1 | Strand | |
| 2686a_s_at | | Exonic) | | | | | [Source: HGNC | | | |
| | | | | | | | Symbol; Acc: 393] | | | |
| OC3P. | Expression | Sense | 11 | ENSG00000- | UST | 10090 | uronyl-2- | Chr | Forward | 613 |
| 12648. | probeset | (Fully | | 111962 | | | sulfotransferase | 6 | Strand | |
| C1_s_at | | Exonic) | | | | | [Source: HGNC | | | |
| | | | | | | | Symbol; Acc: 17223] | | | |
| OC3SNGn. | Expression | Sense | 11 | ENSG00000- | ARL4A | 10124 | ADP-ribosylation | Chr | Forward | 614 |
| 2612- | probeset | (Fully | | 122644 | | | factor-like 4A | 7 | Strand | |
| 800a_s_at | | Exonic) | | | | | [Source: HGNC | | | |
| | | | | | | | Symbol; Acc: 695] | | | |
| OCADA. | Expression | Sense | 11 | ENSG00000- | MBNL2 | 10150 | muscleblind-like splicing | Chr | Forward | 615 |
| 3083_s_at | probeset | (Fully | | 139793 | | | regulator 2 | 13 | Strand | |
| | | Exonic) | | | | | [Source: HGNC | | | |
| | | | | | | | Symbol; Acc: 16746] | | | |
| OCADNP. | Expression | Sense | 11 | ENSG00000- | AASS | 10157 | aminoadipate-semialdehyde | Chr | Reverse | 616 |
| 8513_s_at | probeset | (Fully | | 008311 | | | synthase | 7 | Strand | |
| | | Exonic) | | | | | [Source: HGNC | | | |
| | | | | | | | Symbol; Acc: 17366] | | | |
| OCADA. | Expression | Sense | 11 | ENSG00000- | TRIM13 | 10206 | tripsrtite motif | Chr | Forward | 617 |
| 3572_s_at | probeset | (Fully | | 204977 | | | containing 13 | 13 | Strand | |
| | | Exonic) | | | | | [Source: HGNC | | | |
| | | | | | | | Symbol; Acc: 9976] | | | |
| OC3P. | Expression | Sense | 11 | ENSG00000- | MYL9 | 10398 | myosin, light chain 9, | Chr | Forward | 618 |
| 2537. | probeset | (Fully | | 101335 | | | regulatory | 20 | Strand | |
| CB1_s_at | | Exonic) | | | | | [Source: HGNC | | | |
| | | | | | | | Symbol; Acc: 15754] | | | |
| OCHP. | Expression | Sense | 11 | ENSG00000- | PROCR | 10544 | protein C receptor, | Chr | Forward | 619 |
| 164_s_at | probeset | (Fully | | 101000 | | | endothelial | 20 | Strand | |
| | | Exonic) | | | | | [Source: HGNC | | | |
| | | | | | | | Symbol; Acc: 9452] | | | |
| OC3SNGnh. | Expression | Sense | 11 | ENSG00000- | NUDT4 | 11163 | nudix (nucleoside | Chr | Forward | 620 |
| 17196_s_at | probeset | (Fully | | 173598 | | | diphosphate | 12 | Strand | |
| | | Exonic) | | | | | linked moitey | | | |
| | | | | | | | X)-type motif 4 | | | |
| | | | | | | | [Source: HGNC | | | |
| | | | | | | | Symbol; Acc: 8051] | | | |
| OC3P. | Expression | Sense | 6 | ENSG00000- | FSTL1 | 11167 | follistatin-like 1 | Chr | Reverse | 621 |
| 211. | probeset | (Fully | | 163430 | | | [Source: HGNC | 3 | Strand | |
| C1_x_at | | Exonic) | | | | | Symbol; Acc: | | | |
| | | | | | | | 3972] | | | |
| OCMXSNG. | Expression | Sense | 10 | ENSG00000- | TREH | 11181 | trehalase (brush-border | Chr | Reverse | 622 |
| 5380_x_at | probeset | (Fully | | 118094 | | | membrane glycoprotein | 11 | Strand | |
| | | Exonic) | | | | | [Source: HGNC | | | |
| | | | | | | | Symbol; Acc: 12266] | | | |
| OC3P. | Expression | Sense | 11 | ENSG00000- | GPR176 | 11245 | G protein-coupled | Chr | Reverse | 623 |
| 11725.C1_at | probeset | (Fully | | 166073 | | | receptor 176 | 15 | Strand | |
| | | Exonic) | | | | | [Source: HGNC | | | |
| | | | | | | | Symbol; Acc: 32370] | | | |
| OCMX. | Expression | Sense | 11 | ENSG00000- | KLF12 | 11278 | Kruppel-like | Chr | Reverse | 624 |
| 3883. | probeset | (includes | | 118922 | | | factor 12 | 13 | Strand | |
| C1_s_at | | Intronic) | | | | | [Source: HGNC | | | |
| | | | | | | | Symbol; Acc: 6346] | | | |
| OC3SNG. | Expression | Sense | 11 | ENSG00000- | KLF8 | 11279 | Kruppel-like | Chr | Forward | 625 |
| 3488- | probeset | (Fully | | 102349 | | | factor 8 | X | Strand | |
| 18a_s_at | | Exonic) | | | | | [Source: HGNC | | | |
| | | | | | | | Symbol; Acc: 6351] | | | |
| OC3P. | Expression | Sense | 11 | ENSG00000- | NID2 | 22795 | nidogen 2 | Chr | Reverse | 626 |
| 164. | probeset | (Fully | | 087303 | | | (osteonidogen) | 14 | Strand | |
| C1_s_at | | Exonic) | | | | | [Source: HGNC | | | |
| | | | | | | | Symbol; Acc: 13389] | | | |
| OC3SNG. | Expression | Sense | 11 | ENSG00000- | RHOBTB3 | 22836 | Rho-related BTB domain | Chr | Forward | 627 |
| 2402- | probeset | (Fully | | 164292 | | | containing 3 | 5 | Strand | |
| 2883a_s_at | | Exonic) | | | | | [Source: HGNC | | | |
| | | | | | | | Symbol; Acc: 18757] | | | |
| OC3SNGnh. | Expression | Sense | 11 | ENSG00000- | PLA2R1 | 22925 | phospholipase A2 | Chr | Reverse | 628 |
| 14944_at | probeset | (includes | | 153246 | | | receptor 1, 180 kDa | 2 | Strand | |
| | | Intronic) | | | | | [Source: HGNC | | | |
| | | | | | | | Symbol; Acc: 9042] | | | |
| OC3SNGnh. | Expression | Anti- | 11 | ENSG00000- | DAAM1 | 23002 | dishevelled associated | Chr | Forward | 629 |
| 9821_s_at | probeset | Sense | | 100592 | | | activator of morphogenesis 1 | 14 | Strand | |
| | | | | | | | [Source: HGNC | | | |
| | | | | | | | Symbol; Acc: 18142] | | | |
| OC3P. | Expression | Sense | 11 | ENSG00000- | KIAA0922 | 23240 | KIAA0922 | Chr | Forward | 630 |
| 10089. | probeset | (Fully | | 121210 | | | [Source: HGNC | 4 | Strand | |
| C1_at | | Exonic) | | | | | Symbol; Acc: | | | |
| | | | | | | | 29146] | | | |
| OC3P. | Expression | Sense | 11 | ENSG00000- | PSD3 | 23362 | pleckstrin and Sec7 domain | Chr | Reverse | 631 |
| 11485. | probeset | (Fully | | 156011 | | | containing 3 | 8 | Strand | |
| C1_s_at | | Exonic) | | | | | [Source: HGNC | | | |
| | | | | | | | Symbol; Acc: 19093] | | | |
| OC3P. | Expression | Sense | 11 | ENSG00000- | ANGPTL2 | 23452 | angiopoietin- | Chr | Reverse | 632 |
| 2679. | probeset | (Fully | | 136859 | | | like 2 | 9 | Strand | |
| C1_s_at | | Exonic) | | | | | [Source: HGNC | | | |
| | | | | | | | Symbol; Acc: 490] | | | |
| OC3SNGnh. | Expression | Sense | 11 | ENSG00000- | SPIDR | 23514 | scaffolding protein involved | Chr | Forward | 633 |
| 20422_s_at | probeset | (Fully | | 272972 | | | in DNA repair | HG1699_ | Strand | |
| | | Exonic) | | | | | [Source: HGNC | PATCH | | |
| | | | | | | | Symbol; Acc: 28971] | | | |
| OCADA. | Expression | Sense | 11 | ENSG00000- | RBMS3 | 27303 | RNA binding motif, single | Chr | Forward | 634 |
| 2087_s_at | probeset | (includes | | 144642 | | | stranded interacting protein 3 | 3 | Strand | |
| | | Intronic) | | | | | [Source: HGNC | | | |
| | | | | | | | Symbol; Acc: 13427] | | | |
| OCRS. | Expression | Sense | 11 | ENSG00000- | NOX4 | 50507 | NADPH oxidase 3 | Chr | Reverse | 635 |
| 320_s_at | probeset | (Fully | | 086991 | | | [Source: HGNC | 11 | Strand | |
| | | Exonic) | | | | | Symbol; Acc: | | | |
| | | | | | | | 7891] | | | |
| OC3SNGn. | Expression | Sense | 11 | ENSG00000- | STMN3 | 50861 | stathmin-like 3 | Chr | Reverse | 636 |
| 793- | probeset | (Fully | | 197457 | | | [Source: HGNC | 20 | Strand | |
| 57a_s_at | | Exonic) | | | | | Symbol; Acc: | | | |
| | | | | | | | 15926] | | | |
| OC3P. | Expression | Sense | 11 | ENSG00000- | VPS36 | 51028 | vacuolar protein sorting 36 | Chr | Reverse | 637 |
| 3460. | probeset | (Fully | | 136100 | | | homolog (S. cerevisiae) | 13 | Strand | |
| C1_s_at | | Exonic) | | | | | [Source: HGNC | | | |
| | | | | | | | Symbol; Acc: 20312] | | | |
| OC3P. | Expression | Sense | 11 | ENSG00000- | FAM198B | 51313 | family with sequence | Chr | Reverse | 638 |
| 6417. | probeset | (Fully | | 164125 | | | similarity 198, member B | 4 | Strand | |
| C1_s_at | | Exonic) | | | | | [Source: HGNC | | | |
| | | | | | | | Symbol; Acc: 25312] | | | |
| OC3P. | Expression | Sense | 11 | ENSG00000- | CHST15 | 51363 | carbohydrate (N- | Chr | Reverse | 639 |
| 6659. | probeset | (Fully | | 182022 | | | acetylgalactosamine 4-sulfate | 10 | Strand | |
| C1_s_at | | Exonic) | | | | | 6-O) sulfotransferase 15 | | | |
| | | | | | | | [Source: HGNC | | | |
| | | | | | | | Symbol; Acc: 18137] | | | |
| OC3SNGn. | Expression | Sense | 11 | ENSG00000- | WNT4 | 54361 | wingless-type MMTV | Chr | Reverse | 640 |
| 7890- | probeset | (Fully | | 162552 | | | integration site | 1 | Strand | |
| 859a_x_at | | Exonic) | | | | | family, member 4 | | | |
| | | | | | | | [Source: HGNC | | | |
| | | | | | | | Symbol; Acc: 12783] | | | |
| OC3P. | Expression | Sense | 11 | ENSG00000- | SMOX | 54498 | spermine oxidease | Chr | Forward | 641 |
| 11993. | probeset | (Fully | | 088826 | | | [Source: HGNC | 20 | Strand | |
| C1_s_at | | Exonic) | | | | | Symbol; Acc: | | | |
| | | | | | | | 15862] | | | |
| OC3P. | Expression | Sense | 11 | ENSG00000- | EPDR1 | 54749 | ependymin related 1 | Chr | Forward | 642 |
| 5230. | probeset | (Fully | | 086289 | | | [Source: HGNC | 7 | Strand | |
| C1_s_at | | Exonic) | | | | | Symbol; Acc: | | | |
| | | | | | | | 17572] | | | |
| OC3P. | Expression | Sense | 11 | ENSG00000- | FBLIM1 | 54751 | filamin binding LIM | Chr | Forward | 643 |
| 9910. | probeset | (Fully | | 162458 | | | protein 1 | 1 | Strand | |
| C1_s_at | | Exonic) | | | | | [Source: HGNC | | | |
| | | | | | | | Symbol; Acc: 24686] | | | |
| OC3SNG. | Expression | Sense | 9 | ENSG00000- | TOR4A | 54863 | torsin family 4, | Chr | Forward | 644 |
| 4208- | probeset | (Fully | | 198113 | | | member A | 9 | Strand | |
| 25a_x_at | | Exonic) | | | | | [Source: HGNC | | | |
| | | | | | | | Symbol; Acc: 25981] | | | |
| OCMX. | Expression | Anti- | 11 | ENSG00000- | WHSC1L1 | 54904 | Wolf-Hirschhorn syndrome | Chr | Reverse | 645 |
| 1760. | probeset | Sense | | 147548 | | | candidate 1-like 1 | 8 | Strand | |
| C2_s_at | | | | | | | [Source: HGNC | | | |
| | | | | | | | Symbol; Acc: 12767] | | | |
| OCADNP. | Expression | Sense | 11 | ENSG00000- | MEG3 | 55384 | maternally expressed 3 (non- | Chr | Forward | 646 |
| 12059_s_at | probeset | (Fully | | 214548 | | | protein coding) | 14 | Strand | |
| | | Exonic) | | | | | [Source: HGNC | | | |
| | | | | | | | Symbol; Acc: 14575] | | | |
| OC3P. | Expression | Sense | 11 | ENSG00000- | MEG3 | 55384 | maternally expressed 3 (non- | Chr | Forward | 647 |
| 9532. | probeset | (Fully | | 214548 | | | protein coding) | 14 | Strand | |
| C1_s_at | | Exonic) | | | | | [Source: HGNC | | | |
| | | | | | | | Symbol; Acc: 14575] | | | |
| OC3P. | Expression | Sense | 11 | ENSG00000- | MEG3 | 55384 | maternally expressed 3 (non- | Chr | Forward | 658 |
| 13642. | probeset | (Fully | | 214548 | | | protein coding) | 14 | Strand | |
| C1_s_at | | Exonic) | | | | | [Source: HGNC | | | |
| | | | | | | | Symbol; Acc: 14575] | | | |
| OC3P.2179. | Expression | Sense | 11 | ENSG00000- | SULF2 | 55959 | sulfatase 2 | Chr | Reverse | 649 |
| C1_s_at | probeset | (Fully | | 196562 | | | [Source: HGNC | 20 | Strand | |
| | | Exonic) | | | | | Symbol; Acc: | | | |
| | | | | | | | 20392] | | | |
| OC3P. | Expression | Sense | 10 | ENSG00000- | PDGFC | 56034 | platelet derived growth | Chr | Reverse | 650 |
| 10040. | probeset | (Fully | | 145431 | | | factor C | 4 | Strand | |
| C1_s_at | | Exonic) | | | | | [Source: HGNC | | | |
| | | | | | | | Symbol; Acc: 8801] | | | |
| OCADA. | Expression | Sense | 11 | ENSG00000- | PDGFC | 56034 | platelet derived growth | Chr | Reverse | 651 |
| 1904_s_at | probeset | (includes | | 145431 | | | factor C | 4 | Strand | |
| | | Intronic) | | | | | [Source: HGNC | | | |
| | | | | | | | Symbol; Acc: 8801] | | | |
| OCMXSNG. | Expression | Anti- | 11 | ENSG00000- | TMX4 | 56255 | thioredoxin-related | Chr | Reverse | 652 |
| 126_x_at | probeset | Sense | | 125827 | | | transmembrane protein 4 | 20 | Strand | |
| | | | | | | | [Source: HGNC | | | |
| | | | | | | | Symbol; Acc: 25237] | | | |
| OC3P.1363. | Expression | Sense | 11 | ENSG00000- | NPDC1 | 56654 | neural proliferation, | Chr | Reverse | 653 |
| C1_s_at | probeset | (Fully | | 107281 | | | differentation and control, 1 | 9 | Strand | |
| | | Exonic) | | | | | [Source: HGNC | | | |
| | | | | | | | Symbol; Acc: 25237] | | | |
| OCADA. | Expression | Sense | 11 | ENSG00000- | SPHK2 | 56848 | sphingosine | Chr | Forward | 654 |
| 11214_s_at | probeset | (Fully | | 063176 | | | kinase 2 | 19 | Strand | |
| | | Exonic) | | | | | [Source: HGNC | | | |
| | | | | | | | Symbol; Acc: 18859] | | | |
| OC3P. | Expression | Sense | 11 | ENSG00000- | TMEM159 | 57146 | transmembrane protein 159 | Chr | Forward | 655 |
| 12259. | probeset | (Fully | | 011638 | | | [Source: HGNC | 16 | Strand | |
| C1_x_at | | Exonic) | | | | | Symbol; Acc: | | | |
| | | | | | | | 30136] | | | |
| OC3P. | Expression | Sense | 11 | ENSG00000- | PLEKHG1 | 57480 | pleckstrin homology domain | Chr | Forward | 656 |
| 13855. | probeset | (Fully | | 120278 | | | containing, family G (with | 6 | Strand | |
| C1_s_at | | Exonic) | | | | | RhoGef domain) member 1 | | | |
| | | | | | | | [Source: HGNC | | | |
| | | | | | | | Symbol; Acc: 20884] | | | |
| OC3SNGnh. | Expression | Anti- | 11 | ENSG00000- | PLEKHG1 | 57480 | pleckstrin homology domain | Chr | Forward | 657 |
| 4306_s_at | probeset | Sense | | 120278 | | | containing, family G (with | 6 | Strand | |
| | | | | | | | RhoGef domain) member 1 | | | |
| | | | | | | | [Source: HGNC | | | |
| | | | | | | | Symbol; Acc: 20884] | | | |
| OCADA. | Expression | Anti- | 11 | ENSG00000- | ZNF608 | 57507 | zinc finger | Chr | Reverse | 658 |
| 6529_s_at | probeset | Sense | | 168916 | | | protein 608 | 5 | Strand | |
| | | | | | | | [Source: HGNC | | | |
| | | | | | | | Symbol; Acc: 29238] | | | |
| OC3P. | Expression | Sense | 10 | ENSG00000- | N/A | 57597 | BAH and colied-coil | Chr | Forward | 659 |
| 14264. | probeset | (Fully | | 171282 | | | domain-containing protein 1 | 17 | Strand | |
| C1_s_at | | Exonic) | | | | | [Source: RefSeq | | | |
| | | | | | | | peptide; Acc: | | | |
| | | | | | | | NP_001073988] | | | |
| OC3P. | Expression | Sense | 11 | ENSG00000- | EDA2R | 60401 | ectodysplasin A2 | Chr | Reverse | 660 |
| 13517. | probeset | (Fully | | 131080 | | | receptor | X | Strand | |
| C1_s_at | | Exonic) | | | | | [Source: HGNC | | | |
| | | | | | | | Symbol; Acc: 17756] | | | |
| OCMXSNG. | Expression | Anti- | 11 | ENSG00000- | NFKBIZ | 64332 | nuclear factor of kappa light | Chr | Forward | 661 |
| 274_s_at | probeset | Sense | | 144802 | | | polypeptide gene enhancer in | 3 | Strand | |
| | | | | | | | B-cells inhibitor, zeta | | | |
| | | | | | | | [Source: HGNC | | | |
| | | | | | | | Symbol; Acc: 29805] | | | |
| OC3P. | Expression | Sense | 11 | ENSG00000- | NFKBIZ | 64332 | nuclear factor of kappa light | Chr | Forward | 662 |
| 697. | probeset | (Fully | | 144802 | | | polypeptide gene enhancer in | 3 | Strand | |
| C1_s_at | | Exonic) | | | | | B-cells inhibitor, zeta | | | |
| | | | | | | | [Source: HGNC | | | |
| | | | | | | | Symbol; Acc: 29805] | | | |
| OC3SNG. | Expression | Sense | 11 | ENSG00000- | CSRNP1 | 64651 | cysteine-serine-rich | Chr | Reverse | 663 |
| 3829- | probeset | (Fully | | 144655 | | | nuclear protein 1 | 3 | Strand | |
| 22a_s_at | | Exonic) | | | | | [Source: HGNC | | | |
| | | | | | | | Symbol; Acc: 14300] | | | |
| OC3SNGnh. | Expression | Sense | 11 | ENSG00000- | ZNF655 | 79027/// | zinc finger | Chr | Forward | 664 |
| 17408_s_at | probeset | (Fully | | 197343 | | 101929496 | protein 655 | 7 | Strand | |
| | | Exonic) | | | | | [Source: HGNC | | | |
| | | | | | | | Symbol; Acc: 30899] | | | |
| OCADNP. | Expression | Sense | 11 | ENSG00000- | MEG8 | 79104/// | maternally expressed 8 | Chr | Forward | 665 |
| 13827_s_at | probeset | (includes | | 258399 | | 692215 | (non-protein coding) | 14 | Strand | |
| | | Intronic) | | | | | [Source: HGNC | | | |
| | | | | | | | Symbol; Acc: 14574] | | | |
| OC3P. | Expression | Sense | 11 | ENSG00000- | MEG8 | 79104/// | maternally expressed 8 | Chr | Forward | 666 |
| 12074. | probeset | (includes | | 258399 | | 692215 | (non-protein coding) | 14 | Strand | |
| C1_s_at | | Intronic) | | | | | [Source: HGNC | | | |
| | | | | | | | Symbol; Acc: 14574] | | | |
| OC3SNGnh. | Expression | Sense | 8 | ENSG00000- | TMEM43 | 79188 | transmembrane protein 43 | Chr | Forward | 667 |
| 12321_s_at | probeset | (includes | | 170876 | | | [Source: HGNC | 3 | Strand | |
| | | Intronic) | | | | | Symbol; Acc: | | | |
| | | | | | | | 28472] | | | |
| OCRS2. | Expression | Sense | 11 | ENSG00000- | SPAG16 | 79582 | sperm associated | Chr | Forward | 668 |
| 9432_s_at | probeset | (Fully | | 144451 | | | antigen 16 | 2 | Strand | |
| | | Exonic) | | | | | [Source: HGNC | | | |
| | | | | | | | Symbol; Acc: 23225] | | | |
| OCMX. | Expression | Sense | 11 | ENSG00000- | SNRNP25 | 79622 | small nuclear | Chr | Forward | 669 |
| 13587. | probeset | (Fully | | 161981 | | | ribonucleoprotein | 16 | Strand | |
| C1_s_at | | Exonic) | | | | | 25 kDa (U11/U12) | | | |
| | | | | | | | [Source: HGNC | | | |
| | | | | | | | Symbol; Acc: 14161] | | | |
| OCHP. | Expression | Sense | 11 | ENSG00000- | PTGIS | 5740 | prostaglandin I2 | Chr | Reverse | 670 |
| 179_x_at | probeset | (Fully | | 124212 | | | (prostacyclin) synthase | 20 | Strand | |
| | | Exonic) | | | | | [Source: HGNC | | | |
| | | | | | | | Symbol; Acc: 9603] | | | |
| OCMX. | Expression | Sense | 10 | ENSG00000- | WDR78 | 79819 | WD repeat | Chr | Reverse | 671 |
| 14790. | probeset | (includes | | 152763 | | | domain 78 | 1 | Strand | |
| C1_at | | Intronic) | | | | | [Source: HGNC | | | |
| | | | | | | | Symbol; Acc: 26252] | | | |
| OC3SNGnh. | Expression | Sense | 9 | ENSG00000- | PDGFD | 80310 | platelet derived | Chr | Reverse | 672 |
| 16119_at | probeset | (includes | | 170962 | | | growth factor D | 11 | Strand | |
| | | Intronic) | | | | | [Source: HGNC | | | |
| | | | | | | | Symbol; Acc: 30620] | | | |
| OC3P. | Expression | Sense | 11 | ENSG00000- | SETD7 | 80854 | SET domain containing | Chr | Reverse | 673 |
| 5764. | probeset | (includes | | 145391 | | | (lysine methyltransferase) 7 | 4 | Strand | |
| C1_s_at | | Intronic) | | | | | [Source: HGNC | | | |
| | | | | | | | Symbol; Acc: 30412] | | | |
| OC3P. | Expression | Sense | 11 | ENSG00000- | VMP1 | 81671/// | vacuole membrane | Chr | Forward | 674 |
| 564. | probeset | (includes | | 062716 | | 406991 | protein 1 | 17 | Strand | |
| C1- | | Intronic) | | | | | [Source: HGNC | | | |
| 358a_s_at | | | | | | | Symbol; Acc: 29559] | | | |
| OC3P. | Expression | Sense | 11 | ENSG00000- | VMP1 | 81671/// | vacuole membrane | Chr | Forward | 675 |
| 564. | probeset | (Fully | | 062716 | | 406991 | protein 1 | 17 | Strand | |
| C1_s_at | | Exonic) | | | | | [Source: HGNC | | | |
| | | | | | | | Symbol; Acc: 29559] | | | |
| OC3SNGnh. | Expression | Sense | 11 | ENSG00000- | ZNF611 | 81856 | zinc finger | Chr | Reverse | 676 |
| 4611_s_at | probeset | (Fully | | 213020 | | | protein 611 | 19 | Strand | |
| | | Exonic) | | | | | [Source: HGNC | | | |
| | | | | | | | Symbol; Acc: 28766] | | | |
| OC3P. | Expression | Sense | 11 | ENSG00000- | JAM3 | 83700 | junctional adhesion | Chr | Forward | 677 |
| 560. | probeset | (Fully | | 166086 | | | molecule 3 | 11 | Strand | |
| C1_s_at | | Exonic) | | | | | [Source: HGNC | | | |
| | | | | | | | Symbol; Acc: 15532] | | | |
| OC3P. | Expression | Sense | 11 | ENSG00000- | ANTXR1 | 84168 | anthrax toxin | Chr | Forward | 678 |
| 925. | probeset | (Fully | | 169604 | | | receptor 1 | 2 | Strand | |
| C1_s_at | | Exonic) | | | | | [Source: HGNC | | | |
| | | | | | | | Symbol; Acc: 21014] | | | |
| OCMXSNG. | Expression | Sense | 11 | ENSG00000- | CMSS1 | 84319 | cms1 ribosomal | Chr | Forward | 679 |
| 3100_s_at | probeset | (includes | | 184220 | | | small subunit | 3 | Strand | |
| | | Intronic) | | | | | homolog (yeast) | | | |
| | | | | | | | [Source: HGNC | | | |
| | | | | | | | Symbol; Acc: 28666] | | | |
| OCMX. | Expression | Sense | 11 | ENSG00000- | CMSS1 | 84319 | cms1 ribosomal | Chr | Forward | 680 |
| 3329. | probeset | (includes | | 184220 | | | small subunit | 3 | Strand | |
| C1_s_at | | Intronic) | | | | | homolog (yeast) | | | |
| | | | | | | | [Source: HGNC | | | |
| | | | | | | | Symbol; Acc: 28666] | | | |
| OC3P. | Expression | Sense | 11 | ENSG00000- | HOPX | 84525 | HOP homeobox | Chr | Reverse | 681 |
| 6769. | probeset | (Fully | | 171476 | | | [Source: HGNC | 4 | Strand | |
| C1_s_at | | Exonic) | | | | | Symbol; Acc: 24961] | | | |
| OC3P. | Expression | Sense | 11 | ENSG00000- | FNDC1 | 84624 | fibronectin type III domain | Chr | Forward | 682 |
| 632. | probeset | (Fully | | 164694 | | | containing 1 | 6 | Strand | |
| C1_s_at | | Exonic) | | | | | [Source: HGNC | | | |
| | | | | | | | Symbol; Acc: 21184] | | | |
| OC3P. | Expression | Sense | 11 | ENSG00000- | COL27A1 | 85301 | collagen, type XXVII, | Chr | Forward | 683 |
| 4390. | probeset | (Fully | | 196739 | | | alpha 1 | 9 | Strand | |
| C1_s_at | | Exonic) | | | | | [Source: HGNC | | | |
| | | | | | | | Symbol; Acc: 22986] | | | |
| OC3P. | Expression | Sense | 11 | ENSG00000- | NAV1 | 89796 | neuron navigator 1 | Chr | Forward | 684 |
| 13498. | probeset | (Fully | | 134369 | | | [Source: HGNC | 1 | Strand | |
| C1_s_at | | Exonic) | | | | | Symbol; Acc: 15989] | | | |
| OCADA. | Expression | Sense | 11 | ENSG00000- | TSPAN18 | 90139 | tetraspanin 18 | Chr | Forward | 685 |
| 3141_s_at | probeset | (Fully | | 157570 | | | [Source: HGNC | 11 | Strand | |
| | | Exonic) | | | | | Symbol; Acc: 20660] | | | |
| OC3P. | Expression | Sense | 9 | ENSG00000- | SHF | 90525 | Src homology 2 domain | Chr | Reverse | 686 |
| 13629. | probeset | (Fully | | 138606 | | | containing F | 15 | Strand | |
| C1_s_at | | Exonic) | | | | | [Source: HGNC | | | |
| | | | | | | | Symbol; Acc: 25116] | | | |
| OCADNP. | Expression | Sense | 11 | ENSG00000- | PLXNA4 | 91584 | plexin A4 | Chr | Reverse | 687 |
| 7019_s_at | probeset | (Fully | | 221866 | | | [Source: HGNC | 7 | Strand | |
| | | Exonic) | | | | | Symbol; Acc: 9102] | | | |
| OC3P. | Expression | Sense | 11 | ENSG00000- | FANK1 | 92565 | fibronectin type III and | Chr | Forward | 688 |
| 2860. | probeset | (Fully | | 203780 | | | repeat domains 1 | 10 | Strand | |
| C1_s_at | | Exonic) | | | | | [Source: HGNC | | | |
| | | | | | | | Symbol; Acc: 23527] | | | |
| OC3P. | Expression | Sense | 11 | ENSG00000- | FBXO32 | 114907 | F-box protein 32 | Chr | Reverse | 689 |
| 7638. | probeset | (Fully | | 156804 | | | [Source: HGNC | 8 | Strand | |
| C1_s_at | | Exonic) | | | | | Symbol; Acc: 16731] | | | |
| OCHP. | Expression | Anti- | 8 | ENSG00000- | CTHRC1 | 115908 | collagen triple helix | Chr | Forward | 690 |
| 19_s_at | probeset | Sense | | 164932 | | | repeat containing 1 | 8 | Strand | |
| | | | | | | | [Source: HGNC | | | |
| | | | | | | | Symbol; Acc: 18831] | | | |
| OC3P. | Expression | Sense | 11 | ENSG00000- | ANTXR2 | 118429 | anthrax toxin | Chr | Reverse | 691 |
| 9764. | probeset | (Fully | | 163297 | | | receptor 2 | 4 | Strand | |
| C1_s_at | | Exonic) | | | | | [Source: HGNC | | | |
| | | | | | | | Symbol; Acc: 21732] | | | |
| OC3P. | Expression | Sense | 11 | ENSG00000- | CMTM3 | 123920 | CKLF-like MARVEL | Chr | Forward | 692 |
| 89654. | probeset | (Fully | | 140931 | | | transmembrane domain | 16 | Strand | |
| C1_s_at | | Exonic) | | | | | containing 3[Source: HGNC | | | |
| | | | | | | | Symbol; Acc: 19174] | | | |
| OCADA. | Expression | Sense | 10 | ENSG00000- | FAM69C | 125704 | family with | Chr | Reverse | 693 |
| 12049_s_at | probeset | (Fully | | 187773 | | | sequence similarity | 18 | Strand | |
| | | Exonic) | | | | | 69, member C | | | |
| | | | | | | | [Source: HGNC | | | |
| | | | | | | | Symbol; Acc: 31729] | | | |
| OC3SNG. | Expression | Sense | 11 | ENSG00000- | ISM1 | 140862 | isthmin 1, | Chr | Forward | 694 |
| 3388- | probeset | (Fully | | 101230 | | | angiogenesis inhibitor | 20 | Strand | |
| 17a_s_at | | Exonic) | | | | | [Source: HGNC | | | |
| | | | | | | | Symbol; Acc: 16213] | | | |
| OCADNP. | Expression | Sense | 11 | ENSG00000- | SESN3 | 143686 | sestrin 3 | Chr | Reverse | 695 |
| 13664_s_at | probeset | (Fully | | 149212 | | | [Source: HGNC | 11 | Strand | |
| | | Exonic) | | | | | Symbol; Acc: | | | |
| | | | | | | | 23060] | | | |
| OCHP. | Expression | Sense | 11 | ENSG00000- | APCDD1 | 147495 | adenomatosis polyposis coli | Chr | Forward | 696 |
| 1423_s_at | probeset | (Fully | | 154856 | | | down-regulated 1 | 18 | Strand | |
| | | Exonic) | | | | | [Source: HGNC | | | |
| | | | | | | | Symbol; Acc: 15718] | | | |
| OC3SNGn. | Expression | Sense | 11 | ENSG00000- | ZNF548 | 147694 | zinc finger | Chr | Forward | 697 |
| 3144- | probeset | (Fully | | 188785 | | | protein 548 | 19 | Strand | |
| 297a_s_at | | Exonic) | | | | | [Source: HGNC | | | |
| | | | | | | | Symbol; Acc: 26561] | | | |
| OC3P. | Expression | Sense | 11 | ENSG00000- | N/A | 150967 | NOVEL | Chr | Forward | 698 |
| 14266. | probeset | (Fully | | 260804 | | | lincRNA | 2 | Strand | |
| C1-547a_ | | Exonic) | | | | | (Clone_based_ | | | |
| s_at | | | | | | | vega_gene) | | | |
| OC3SNG. | Expression | Sense | 11 | ENSG00000- | CCDC80 | 151887 | coiled-coil domain | Chr | Reverse | 699 |
| 5645- | probeset | (Fully | | 091986 | | | containing 80 | 3 | Strand | |
| 98a_x_at | | Exonic) | | | | | [Source: HGNC | | | |
| | | | | | | | Symbol; Acc: 30649] | | | |
| OC3P. | Expression | Sense | 11 | ENSG00000- | ZNF827 | 152485//// | zinc finger | Chr | Reverse | 700 |
| 14403. | probeset | (Fully | | 151612 | | 101927707 | protein 827 | 4 | Strand | |
| C1_s_at | | Exonic) | | | | | [Source: HGNC | | | |
| | | | | | | | Symbol; Acc: 27193] | | | |
| OC3SNGn. | Expression | Sense | 11 | ENSG00000- | AMOTL1 | 154810 | angiomotin | Chr | Forward | 701 |
| 538- | probeset | (Fully | | 166025 | | | like 1 | 11 | Strand | |
| 5592a_s_at | | Exonic) | | | | | [Source: HGNC | | | |
| | | | | | | | Symbol; Acc: 17811] | | | |
| OCRS. | Expression | Sense | 11 | ENSG00000- | KANK4 | 163782 | KN motif and ankyrin | Chr | Reverse | 702 |
| 115_s_at | probeset | (Fully | | 132854 | | | repeat domains 3 | 1 | Strand | |
| | | Exonic) | | | | | [Source: HGNC | | | |
| | | | | | | | Symbol; Acc: 27263] | | | |
| OC3P. | Expression | Sense | 11 | ENSG00000- | PRICK- | 166336 | prickle homolog | Chr | Reverse | 703 |
| 5913. | probeset | (Fully | | 163637 | LE2 | | 2 (Drosophila) | 3 | Strand | |
| C1_s_at | | Exonic) | | | | | [Source: HGNC | | | |
| | | | | | | | Symbol; Acc: 27193] | | | |
| OC3SNGnh. | Expression | Sense | 9 | ENSG00000- | CCDC71L | 168455 | coiled-coil domain | Chr | Reverse | 704 |
| 4002_s_at | probeset | (Fully | | 253276 | | | containing 71-like | 7 | Strand | |
| | | Exonic) | | | | | [Source: HGNC | | | |
| | | | | | | | Symbol; Acc: 26685] | | | |
| OCADA. | Expression | Sense | 9 | ENSG00000- | SYNPO2 | 171024 | synaptopodin | Chr | Forward | 705 |
| 12049_s_at | probeset | (includes | | 172403 | | | 2 [Source: HGNC | 4 | Strand | |
| | | Intronic) | | | | | Symbol; Acc: 17732] | | | |
| OC3SNGnh. | Expression | Sense | 11 | ENSG00000- | SDK1 | 221935 | sidekick cell adhesion | Chr | Forward | 706 |
| 4002_s_at | probeset | (includes | | 146555 | | | molecule 1 | 7 | Strand | |
| | | Intronic) | | | | | [Source: HGNC | | | |
| | | | | | | | Symbol; Acc: 19307] | | | |
| OC3SNGnh. | Expression | Sense | 11 | ENSG00000- | MTURN | 222166 | maturin, neural progenitor | Chr | Forward | 707 |
| 4002_s_at | probeset | (Fully | | 180354 | | | differentation regulator | 7 | Strand | |
| | | Exonic) | | | | | homolog (Xenopus) | | | |
| | | | | | | | [Source: HGNC | | | |
| | | | | | | | Symbol; Acc: 25457] | | | |
| OC3P. | Expression | Sense | 11 | ENSG00000- | SERINC5 | 256987 | serine incorporator | Chr | Reverse | 708 |
| 11044. | probeset | (includes | | 164300 | | | 5 | 5 | Strand | |
| C1_at | | Intronic) | | | | | [Source: HGNC | | | |
| | | | | | | | Symbol; Acc: 18825] | | | |
| OC3P. | Expression | Sense | 10 | ENSG00000- | SERINC5 | 256987 | serine incorporator | Chr | Reverse | 709 |
| 11044. | probeset | (includes | | 164300 | | | 5 | 5 | Strand | |
| C1_x_at | | Intronic) | | | | | [Source: HGNC | | | |
| | | | | | | | Symbol; Acc: 18825] | | | |
| OCMX. | Expression | Sense | 10 | ENSG00000- | SERINC5 | 256987 | serine incorporator | Chr | Reverse | 710 |
| 11593. | probeset | (includes | | 164300 | | | 5 | 5 | Strand | |
| C1_at | | Intronic) | | | | | [Source: HGNC | | | |
| | | | | | | | Symbol; Acc: 18825] | | | |
| OC3SNG. | Expression | Sense | 11 | ENSG00000- | PTRF | 284119 | polymerase I and transcript | Chr | Reverse | 711 |
| 1416- | probeset | (Fully | | 177469 | | | release factor | 17 | Strand | |
| 18a_s_at | | Exonic) | | | | | [Source: HGNC | | | |
| | | | | | | | Symbol; Acc: 9688] | | | |
| OC3P. | Expression | Sense | 11 | ENSG00000- | EMC10 | 284361 | ER membrane protein | Chr | Forward | 712 |
| 603.C1_at | probeset | (Fully | | 161671 | | | complex subunit 10 | 19 | Strand | |
| | | Exonic) | | | | | [Source: HGNC | | | |
| | | | | | | | Symbol; Acc: 27609] | | | |
| OC3SNGnh. | Expression | Sense | 11 | ENSG00000- | RNF149 | 284996 | ring finger | Chr | Reverse | 713 |
| 20169_s_at | probeset | (Fully | | 163162 | | | protein 149 | 2 | Strand | |
| | | Exonic) | | | | | [Source: HGNC | | | |
| | | | | | | | Symbol; Acc: 23137] | | | |
| OCRS2. | Expression | Sense | 11 | ENSG00000- | CYP7C1 | 339761 | cytochrome P450, family 27, | Chr | Reverse | 714 |
| 12918_s_at | probeset | (Fully | | 186684 | | | subfamily C, polypeptide 1 | 2 | Strand | |
| | | Exonic) | | | | | [Source: HGNC | | | |
| | | | | | | | Symbol; Acc: 33480] | | | |
| OC3P. | Expression | Sense | 11 | ENSG00000- | FAM101B | 359845 | family with sequence | Chr | Reverse | 715 |
| 8169. | probeset | (Fully | | 183688 | | | similarity 101, member B | 17 | Strand | |
| C1_s_at | | Exonic) | | | | | [Source: HGNC | | | |
| | | | | | | | Symbol; Acc: 28705 | | | |
| OC3P. | Expression | Sense | 11 | ENSG00000- | PTCHD3P1 | 387647 | patched domain containing | Chr | Forward | 716 |
| 536. | probeset | (includes | | 224597 | | | 3 pseudogene 1 | 10 | Strand | |
| C4_x_at | | Intronic) | | | | | [Source: HGNC | | | |
| | | | | | | | Symbol; Acc: 44945] | | | |
| OCADNP. | Expression | Sense | 8 | ENSG00000- | CREB5 | 9586/// | cAMP responsive element | Chr | Forward | 717 |
| 9287_s_at | probeset | (Fully | | 146592 | | 401317 | binding protein 5 | 7 | Strand | |
| | | Exonic) | | | | | [Source: HGNC | | | |
| | | | | | | | Symbol; Acc: 16844] | | | |
| OCADNP. | Expression | Sense | 11 | ENSG00000- | CREB5 | 9586/// | cAMP responsive element | Chr | Forward | 718 |
| 7150_s_at | probeset | (Fully | | 146592 | | 401317 | binding protein 5 | 7 | Strand | |
| | | Exonic) | | | | | [Source: HGNC | | | |
| | | | | | | | Symbol; Acc: 16844] | | | |
| OC3P. | Expression | Sense | 11 | ENSG00000- | VMP1 | 81671/// | vacuole membrane | Chr | Forward | 719 |
| 2409. | probeset | (Fully | | 062716 | | 406991 | protein 1 | 17 | Strand | |
| C1_s_at | | Exonic) | | | | | [Source: HGNC | | | |
| | | | | | | | Symbol; Acc: 29559] | | | |
| OC3P. | Expression | Sense | 11 | ENSG00000- | MIR22HG | 84981/// | MIR22 host gene | Chr | Reverse | 720 |
| 6031. | probeset | (Fully | | 186594 | | 407004 | (non-protein coding) | 17 | Strand | |
| C1_s_at | | Exonic) | | | | | [Source: HGNC | | | |
| | | | | | | | Symbol; Acc: 28219] | | | |
| OCRS2. | Expression | Sense | 11 | ENSG00000- | MIR22HG | 84981/// | MIR22 host gene | Chr | Reverse | 721 |
| 7332_s_at | probeset | (Fully | | 186594 | | 407004 | (non-protein coding) | 17 | Strand | |
| | | Exonic) | | | | | [Source: HGNC | | | |
| | | | | | | | Symbol; Acc: 28219] | | | |
| OC3SNGnh. | Expression | Sense | 10 | ENSG00000- | MIR31HG | 554202 | MIR31 host gene | Chr | Reverse | 722 |
| 18279_x_at | probeset | (includes | | 171889 | | | (non-protein coding) | 9 | Strand | |
| | | Intronic) | | | | | [Source: HGNC | | | |
| | | | | | | | Symbol; Acc: 37187] | | | |
| OC3SNGnh. | Expression | Sense | 11 | ENSG00000- | MIR31HG | 554202 | MIR31 host gene | Chr | Reverse | 723 |
| 18279_at | probeset | (includes | | 171889 | | | (non-protein coding) | 9 | Strand | |
| | | Intronic) | | | | | [Source: HGNC | | | |
| | | | | | | | Symbol; Acc: 37187] | | | |
| OCADA. | Expression | Sense | 11 | ENSG00000- | ASAH2B | 653308 | N-acylsphingosine | Chr | Forward | 724 |
| 3940_s_at | probeset | (Fully | | 204147 | | | amidohydrolase (non- | 10 | Strand | |
| | | Exonic) | | | | | lysosomal ceramidase) 2B | | | |
| | | | | | | | [Source: HGNC | | | |
| | | | | | | | Symbol; Acc: 23456] | | | |
| OC3SNGnh. | Expression | Sense | 11 | ENSG00000- | GXYLT2 | 727936 | glucoside | Chr | Forward | 725 |
| 7474_s_at | probeset | (includes | | 172986 | | | xylosyltransferase 2 | 3 | Strand | |
| | | Intronic) | | | | | [Source: HGNC | | | |
| | | | | | | | Symbol; Acc: 33383] | | | |
| OCADA. | Expression | Sense | 11 | ENSG00000- | GXYLT2 | 727936 | glucoside | Chr | Forward | 726 |
| 9028_s_at | probeset | (Fully | | 172986 | | | xylosyltransferase 2 | 3 | Strand | |
| | | Exonic) | | | | | [Source: HGNC | | | |
| | | | | | | | Symbol; Acc: 33383] | | | |
| OC3SNGnh. | Expression | Anti- | 11 | ENSG00000- | GXYLT2 | 727936 | glucoside | Chr | Forward | 727 |
| 6774_x_at | probeset | Sense | | 172986 | | | xylosyltransferase 2 | 3 | Strand | |
| | | | | | | | [Source: HGNC | | | |
| | | | | | | | Symbol; Acc: 33383] | | | |
| OC3P. | Expression | Anti- | 11 | ENSG00000- | N/A | 728448 | NOVEL antisense | Chr | Reverse | 728 |
| 2742.C1_at | probeset | Sense | | 182109 | | | (Clone_based_ | 1 | Strand | |
| | | | | | | | vega_gene) | | | |
| OC3P. | Expression | Sense | 11 | ENSG00000- | RELL1 | 768211 | RELT-like 1 | Chr | Reverse | 729 |
| 11717. | probeset | (includes | | 181826 | | | [Source: HGNC | 4 | Strand | |
| C1_s_at | | Intronic) | | | | | Symbol; Acc: 27379] | | | |
| OCRS2. | Expression | Sense | 11 | ENSG00000- | SNHG14 | 3653/// | small nuclear RNA | Chr | Forward | 730 |
| 12554_x_at | probeset | (Fully | | 224078 | | 91380/// | host gene | 15 | Strand | |
| | | Exonic) | | | | 347746/// | 14 (non-protein coding) | | | |
| | | | | | | 100033416/// | [Source: HGNC | | | |
| | | | | | | 100033433/// | Symbol; Acc: 37462] | | | |
| | | | | | | 100033444/// | | | | |
| | | | | | | 100033450/// | | | | |
| | | | | | | 100033802/// | | | | |
| | | | | | | 100033820/// | | | | |
| | | | | | | 101930404 | | | | |
| OCRS2. | Expression | Sense | 11 | ENSG00000- | SNORD- | 100033420 | small nuclear RNA, | Chr | Forward | 731 |
| 10334_x_at | probeset | (Fully | | 207093 | 116-8 | | C/D box 116-8 | 15 | Strand | |
| | | Exonic) | | | | | [Source: HGNC | | | |
| | | | | | | | Symbol; Acc: 33074] | | | |
| OCRS2. | Expression | Sense | 11 | ENSG00000- | SNORD- | 100033434 | small nuclear RNA, | Chr | Forward | 732 |
| 11097_x_at | probeset | (Fully | | 207375 | 116-23 | | C/D box 116-23 | 15 | Strand | |
| | | Exonic) | | | | | [Source: HGNC | | | |
| | | | | | | | Symbol; Acc: 33089] | | | |
| OCMXSNG. | Expression | Sense | 11 | ENSG00000- | MRVI1- | 100129827 | MRVI1 antisense | Chr | Forward | 733 |
| 4302_at | probeset | (includes | | 177112 | AS1 | | RNA 1 | 11 | Strand | |
| | | Intronic) | | | | | [Source: HGNC | | | |
| | | | | | | | Symbol; Acc: 43434] | | | |
| OCADA. | Expression | Anti- | 11 | ENSG00000- | FAM115A | 9747/// | family with sequence | Chr | Reverse | 734 |
| 10563_s_at | probeset | Sense | | 198420 | | 100294033/// | similarity 115, member A | 7 | Strand | |
| | | | | | | 101930003 | [Source: HGNC | | | |
| | | | | | | | Symbol; Acc: 22201] | | | |
| OCMXSNG. | Expression | Anti- | 11 | ENSG00000- | FAM115A | 9747/// | family with sequence | Chr | Reverse | 735 |
| 3737_s_at | probeset | Sense | | 198420 | | 100294033/// | similarity 115, member A | 7 | Strand | |
| | | | | | | 101930003 | [Source: HGNC | | | |
| | | | | | | | Symbol; Acc: 22201] | | | |
| OC3P. | Expression | Sense | 11 | ENSG00000- | CERCAM | 51148 | cerebral endothelial cell | Chr | Forward | 736 |
| 2278. | probeset | (Fully | | 167123 | | | adhesion molecule | 9 | Strand | |
| C1_x_at | | Exonic) | | | | | [Source: HGNC | | | |
| | | | | | | | Symbol; Acc: 23723] | | | |
| OC3P. | Expression | Sense | 11 | ENSG00000- | SNHG14 | 3653/// | small nuclear RNA | Chr | Forward | 737 |
| 10147. | probeset | (Fully | | 224078 | | 91380/// | host gene | 15 | Strand | |
| C1_s_at | | Exonic) | | | | 347746/// | 14 (non-protein coding) | | | |
| | | | | | | 100033416/// | [Source: HGNC | | | |
| | | | | | | 100033433/// | Symbol; Acc: 37462] | | | |
| | | | | | | 100033444/// | | | | |
| | | | | | | 100033450/// | | | | |
| | | | | | | 100033802/// | | | | |
| | | | | | | 100033820/// | | | | |
| | | | | | | 101930404 | | | | |
| OCMX. | Expression | Sense | 11 | ENSG00000- | CA13 | 377677/// | carbonic | Chr | Forward | 738 |
| 5842. | probeset | (Fully | | 185015 | | 100507258 | anhydrase XIII | 8 | Strand | |
| C2_s_at | | Exonic) | | | | | [Source: HGNC | | | |
| | | | | | | | Symbol; Acc: 14914] | | | |
| OC3SNGn. | Expression | Sense | 11 | ENSG00000- | FSCN1 | 6624 | fascin homolog 1, actin- | Chr | Forward | 739 |
| 812- | probeset | (Fully | | 075618 | | | bundling protein | 7 | Strand | |
| 49a_s_at | | Exonic) | | | | | (Strongylocentrotus | | | |
| | | | | | | | pupuratus) [Source: HGNC | | | |
| | | | | | | | Symbol; Acc: 11148] | | | |
| OCMXSNG. | Expression | Anti- | 11 | ENSG00000- | FSCN1 | 6624 | fascin homolog 1, actin- | Chr | Forward | 740 |
| 5099_s_at | probeset | Sense | | 075618 | | | bundling protein | 7 | Strand | |
| | | | | | | | (Strongylocentrotus | | | |
| | | | | | | | pupuratus) [Source: HGNC | | | |
| | | | | | | | Symbol; Acc: 11148] | | | |
| OCMXSNG. | Expression | Sense | 11 | ENSG00000- | N/A | N/A | NOVEL antisense | Chr | Forward | 741 |
| 5706_at | probeset | (Fully | | 254528 | | | (Clone_based_vega_gene) | 11 | Strand | |
| | | Exonic) | | | | | | | | |
| OCMXSNG. | Expression | Sense | 11 | ENSG00000- | N/A | N/A | NOVEL antisense | Chr | Forward | 742 |
| 5706_x_at | probeset | (Fully | | 254528 | | | (Clone_based_vega_gene) | 11 | Strand | |
| | | Exonic) | | | | | | | | |
| OC3P. | Expression | Sense | 8 | ENSG00000- | N/A | N/A | NOVEL sense_overlapping | Chr | Forward | 743 |
| 8258.C1_at | probeset | (Fully | | 260822 | | | (Clone_based_vega_gene) | X | Strand | |
| | | Exonic) | | | | | | | | |
| OCRS2. | Expression | No | 0 | 0 | 0 | 0 | 0 | 0 | 0 | 744 |
| 1506_s_at | probeset | trans- | | | | | | | | |
| | | cription | | | | | | | | |
| | | match | | | | | | | | |
| OCHPRC. | Expression | Sense | 11 | ENSG00000- | N/A | N/A | NOVEL pseudogene | Chr | Reverse | 745 |
| 112_s_at | probeset | (Fully | | 183531 | | | (Clone_based_ensembl_ | 22 | Strand | |
| | | Exonic) | | | | | gene) | | | |
| OC3SNGnh. | Expression | Sense | 11 | ENSG00000- | CASC15 | 401237 | cancer susceptibility | Chr | Forward | 746 |
| 8868_at | probeset | (includes | | 272168 | | | candidate | 6 | Strand | |
| | | Intronic) | | | | | 15 (non-protein coding) | | | |
| | | | | | | | [Source: HGNC | | | |
| | | | | | | | Symbol; Acc: 28245] | | | |
| OC3P.8889. | Expression | Sense | 11 | ENSG00000- | VSTM4 | 196740 | V-set and transmembrane | Chr | Reverse | 747 |
| C1_s_at | probeset | (Fully | | 165633 | | | domain containing 4 | 10 | Strand | |
| | | Exonic) | | | | | [Source: HGNC | | | |
| | | | | | | | Symbol; Acc: 26470] | | | |
| OC3SNGnh. | Expression | Sense | 11 | ENSG00000- | MEG3 | 55384 | maternally expressed 3) | Chr | Forward | 748 |
| 8417_s_at | probeset | (Fully | | 214548 | | | non-protein coding) | 14 | Strand | |
| | | Exonic) | | | | | [Source: HGNC | | | |
| | | | | | | | Symbol; Acc: 14575] | | | |
| OC3P.5448. | Expression | No | 0 | 0 | 0 | 0 | 0 | 0 | 0 | 749 |
| C1_s_at | probeset | trans- | | | | | | | | |
| | | cription | | | | | | | | |
| | | match | | | | | | | | |
| OCADA. | Expression | Sense | 11 | ENSG00000- | IRS1 | 3667 | insulin receptor | Chr | Reverse | 750 |
| 8421_s_at | probeset | (Fully | | 169047 | | | substrate 1 | 2 | Strand | |
| | | Exonic) | | | | | [Source: HGNC | | | |
| | | | | | | | Symbol; Acc: 6125] | | | |
| OCRS2. | Expression | No | 0 | 0 | 0 | 0 | 0 | 0 | 0 | 751 |
| 7772_s_at | probeset | trans- | | | | | | | | |
| | | cription | | | | | | | | |
| | | match | | | | | | | | |
| OC3P.13444. | Expression | Sense | 11 | ENSG00000- | ARL3 | 403 | ADP-ribosylation | Chr | Reverse | 752 |
| C1_s_at | probeset | (Fully | | 138175 | | | factor-like 3 | 10 | Strand | |
| | | Exonic) | | | | | [Source: HGNC | | | |
| | | | | | | | Symbol; Acc: 694] | | | |
| OCADNP. | Expression | Sense | 11 | ENSG00000- | MEG3 | 55384 | maternally expressed 3) | Chr | Forward | 753 |
| 12075_ | probeset | (Fully | | 214548 | | | non-protein coding) | 14 | Strand | |
| s_at | | Exonic) | | | | | [Source: HGNC | | | |
| | | | | | | | Symbol; Acc: 14575] | | | |
| OC3P.13419. | Expression | No | 0 | 0 | 0 | 0 | 0 | 0 | 0 | 754 |
| C1_x_at | probeset | trans- | | | | | | | | |
| | | cription | | | | | | | | |
| | | match | | | | | | | | |
| OCRS2. | Expression | No | 0 | 0 | 0 | 0 | 0 | 0 | 0 | 755 |
| 7772_x_at | probeset | trans- | | | | | | | | |
| | | cription | | | | | | | | |
| | | match | | | | | | | | |
| OCRS2. | Expression | Sense | 11 | ENSG00000- | AQP7P3 | N/A | aquaporin 7 | Chr | Forward | 756 |
| 1905_x_at | probeset | (Fully | | 156750 | | | pseudogene 3 | 9 | Strand | |
| | | Exonic) | | | | | [Source: HGNC | | | |
| | | | | | | | Symbol; Acc: 31976] | | | |
| OC3SNGnh. | Expression | No | 0 | 0 | 0 | 0 | 0 | 0 | 0 | 757 |
| 20450_at | probeset | trans- | | | | | | | | |
| | | cription | | | | | | | | |
| | | match | | | | | | | | |
| OCEM.237_ | Expression | Sense | 11 | ENSG00000- | N/A | N/A | NOVEL antisense | Chr | Reverse | 758 |
| x_at | probeset | (includes | | 258026 | | | (Clone_based_vega_gene) | 12 | Strand | |
| | | Intronic) | | | | | | | | |
| Oc3P. | Expression | No | 0 | 0 | 0 | 0 | 0 | 0 | 0 | 759 |
| 2858. | probeset | trans- | | | | | | | | |
| C1_s_at | | cription | | | | | | | | |
| | | match | | | | | | | | |
| OC3SNGn. | Expression | Anti- | 11 | ENSG00000- | N/A | N/A | NOVEL antisense | Chr | Forward | 760 |
| 1420- | probeset | Sense | | 225032 | | | (Clone_based_vega_gene) | 9 | Strand | |
| 1337a_s_at | | | | | | | | | | |
| OC3SNGn. | Expression | No | 0 | 0 | 0 | 0 | 0 | 0 | 0 | 761 |
| 5831- | probeset | trans- | | | | | | | | |
| 326a_s_at | | cription | | | | | | | | |
| | | match | | | | | | | | |
| OCMXSNG. | Expression | Insu- | 0 | 0 | 0 | 0 | 0 | 0 | 0 | 762 |
| 2371_x_at | probeset | fficient | | | | | | | | |
| | | probes | | | | | | | | |
| | | (<6) | | | | | | | | |
| OCMXSNG. | Expression | Insu- | 0 | 0 | 0 | 0 | 0 | 0 | 0 | 763 |
| 2411_x_at | probeset | fficient | | | | | | | | |
| | | probes | | | | | | | | |
| | | (<6) | | | | | | | | |
| OC3SNGnh. | Expression | Insu- | 0 | 0 | 0 | 0 | 0 | 0 | 0 | 764 |
| 14683_ | probeset | fficient | | | | | | | | |
| x_at | | probes | | | | | | | | |
| | | (<6) | | | | | | | | |
| OC3SNGnh. | Expression | Insu- | 0 | 0 | 0 | 0 | 0 | 0 | 0 | 765 |
| 13886_ | probeset | fficient | | | | | | | | |
| x_at | | probes | | | | | | | | |
| | | (<6) | | | | | | | | |
| OCMXSNG. | Expression | No | 0 | 0 | 0 | 0 | 0 | 0 | 0 | 766 |
| 2415_x_at | probeset | Genome | | | | | | | | |
| | | match | | | | | | | | |
| OC3SNGnh. | Expression | Insu- | 0 | 0 | 0 | 0 | 0 | 0 | 0 | 767 |
| 9044_ | probeset | fficient | | | | | | | | |
| x_at | | probes | | | | | | | | |
| | | (<6) | | | | | | | | |
| OCMXSNG. | Expression | No | 0 | 0 | 0 | 0 | 0 | 0 | 0 | 768 |
| 2681_x_at | probeset | Genome | | | | | | | | |
| | | match | | | | | | | | |
| OCMXSNG. | Expression | Insu- | 0 | 0 | 0 | 0 | 0 | 0 | 0 | 769 |
| 4333_at | probeset | fficient | | | | | | | | |
| | | probes | | | | | | | | |
| | | (<6) | | | | | | | | |
| OC3SNGnh. | Expression | Insu- | 0 | 0 | 0 | 0 | 0 | 0 | 0 | 770 |
| 9044_at | probeset | fficient | | | | | | | | |
| | | probes | | | | | | | | |
| | | (<6) | | | | | | | | |
| OCMXSNG. | Expression | No | 0 | 0 | 0 | 0 | 0 | 0 | 0 | 771 |
| 3121_at | probeset | Genome | | | | | | | | |
| | | match | | | | | | | | |
| OC3SNGnh. | Expression | Insu- | 0 | 0 | 0 | 0 | 0 | 0 | 0 | 772 |
| 9562_x_at | probeset | fficient | | | | | | | | |
| | | probes | | | | | | | | |
| | | (<6) | | | | | | | | |
| OC3SNGnh. | Expression | Insu- | 0 | 0 | 0 | 0 | 0 | 0 | 0 | 773 |
| 6940_x_at | probeset | fficient | | | | | | | | |
| | | probes | | | | | | | | |
| | | (<6) | | | | | | | | |
| OCADA. | Expression | Insu- | 0 | 0 | 0 | 0 | 0 | 0 | 0 | 774 |
| 10862_s_at | probeset | fficient | | | | | | | | |
| | | probes | | | | | | | | |
| | | (<6) | | | | | | | | |
| OCMXSNG. | Expression | Anti- | 11 | ENSG00000- | ZNF117 | 51351 | zinc finger | Chr | Reverse | 775 |
| 2184_at | probeset | Sense | | 152926 | | | protein 117 | 7 | Strand | |
| | | | | | | | [Source: HGNC | | | |
| | | | | | | | Symbol; Acc: 12897] | | | |
| OC3P. | Expression | Sense | 10 | ENSG00000- | MEIS3 | 56917 | Meis homeobox 3 | Chr | Reverse | 776 |
| 3104. | probeset | (Fully | | 105419 | | | [Source: HGNC | 19 | Strand | |
| C1_s_at | | Exonic) | | | | | Symbol; Acc: 29537] | | | |
| OCRS2. | Expression | Sense | 11 | ENSG00000- | SNORD- | 100033417/// | small nucleolar RNA, | Chr | Forward | 777 |
| 11268_x_at | probeset | (Fully | | 207133 | 116-7 | 100033419 | C/D box 116-7 | 15 | Strand | |
| | | Exonic) | | | | | [Source: HGNC | | | |
| | | | | | | | Symbol; Acc: 33073] | | | |
| OCRS2. | Expression | Sense | 11 | ENSG00000- | SNORD- | 100033415/// | small nucleolar RNA, | Chr | Forward | 778 |
| 3193_x_at | probeset | (Fully | | 207014 | 116-3 | 100033421 | C/D box 116-3 | 15 | Strand | |
| | | Exonic) | | | | | [Source: HGNC | | | |
| | | | | | | | Symbol; Acc: 33069] | | | |
| OCMXSNG. | Expression | Sense | 9 | ENSG00000- | CES1P1 | 51716 | carboxylesterase 1 | Chr | Forward | 779 |
| 2287- | probeset | (Fully | | 228595 | | | pseudogene 1 | 16 | Strand | |
| 17a_s_at | | Exonic) | | | | | [Source: HGNC | | | |
| | | | | | | | Symbol; Acc: 18546] | | | |
| OCMXSNG. | Expression | Anti- | 6 | ENSG00000- | INS- | 723961 | INS-IGF2 | Chr | Reverse | 780 |
| 2402_x_at | probeset | Sense | | 129965 | IGF2 | | readthrough | 11 | Strand | |
| | | | | | | | [Source: HGNC | | | |
| | | | | | | | Symbol; Acc: 33527] | | | |
| OCMXSNG. | Expression | Anti- | 11 | ENSG00000- | INS- | 723961 | INS-IGF2 | Chr | Reverse | 781 |
| 2377_x_at | probeset | Sense | | 129965 | IGF2 | | readthrough | 11 | Strand | |
| | | | | | | | [Source: HGNC | | | |
| | | | | | | | Symbol; Acc: 33527] | | | |
| OCMXSNG. | Expression | Anti- | 11 | ENSG00000- | INS- | 723961 | INS-IGF2 | Chr | Reverse | 782 |
| 5242_x_at | probeset | Sense | | 129965 | IGF2 | | readthrough | 11 | Strand | |
| | | | | | | | [Source: HGNC | | | |
| | | | | | | | Symbol; Acc: 33527] | | | |
| OCHPRC. | Expression | Anti- | 11 | ENSG00000- | INS- | 723961 | INS-IGF2 | Chr | Reverse | 783 |
| 192_at | probeset | Sense | | 129965 | IGF2 | | readthrough | 11 | Strand | |
| | | | | | | | [Source: HGNC | | | |
| | | | | | | | Symbol; Acc: 33527] | | | |
| OCEM. | Expression | Anti- | 11 | ENSG00000- | PEG3 | 5178 | paternally | Chr | Reverse | 784 |
| 1215_s_at | probeset | Sense | | 198300 | | | expressed 3 | 19 | Strand | |
| | | | | | | | [Source: HGNC | | | |
| | | | | | | | Symbol; Acc: 8826] | | | |
| OCMXSNG. | Expression | Anti- | 11 | ENSG00000- | ZBED6CL | 113763 | ZBED6 | Chr | Forward | 785 |
| 4986_s_at | probeset | Sense | | 188707 | | | C-terminal like | 7 | Strand | |
| | | | | | | | [Source: HGNC | | | |
| | | | | | | | Symbol; Acc: 21720] | | | |
| OC3SNGnh. | Expression | Sense | 11 | ENSG00000- | INS- | 723961 | INS-IGF2 | Chr | Reverse | 786 |
| 19773_s_at | probeset | (Fully | | 129965 | IGF2 | | readthrough | 11 | Strand | |
| | | Exonic) | | | | | [Source: HGNC | | | |
| | | | | | | | Symbol; Acc: 33527] | | | |
| OC3SNGn. | Expression | Sense | 11 | ENSG00000- | ZNF154 | 7710 | zinc finger | Chr | Reverse | 787 |
| 3705- | probeset | (Fully | | 179909 | | | protein 154 | 19 | Strand | |
| 202a_s_at | | Exonic) | | | | | [Source: HGNC | | | |
| | | | | | | | Symbol; Acc: 12939] | | | |
| OCMXSNG. | Expression | Anti- | 11 | ENSG00000- | EIF2B5 | 8893 | eukaryotic translation | Chr | Forward | 788 |
| 1883_x_at | probeset | Sense | | 145191 | | | initiation factor 2B, | 3 | Strand | |
| | | | | | | | subunit 5 epsilon, | | | |
| | | | | | | | 82 kDa [Source: HGNC | | | |
| | | | | | | | Symbol; Acc: 3261] | | | |

TABLE 12
Subtype analysis: 15 Gene Signature

| GeneID | EntrezGeneID | AUC | ANOVA (p value) | C-Index | LCI C-Index | UCI C-Index |
|---|---|---|---|---|---|---|
| CDH11 | 1009 | 0.851 | 1.35E−25 | 0.558 | 0.515 | 0.596 |
| RAB31 | 11031 | 0.816 | 1.41E−22 | 0.543 | 0.501 | 0.58 |
| COL5A1 | 1289 | 0.815 | 9.01E−22 | 0.572 | 0.534 | 0.602 |
| COL10A1 | 1300 | 0.844 | 2.30E−25 | 0.559 | 0.515 | 0.596 |
| VCAN | 1462 | 0.845 | 1.01E−23 | 0.575 | 0.533 | 0.61 |
| FAP | 2191 | 0.849 | 1.17E−26 | 0.577 | 0.536 | 0.614 |
| FN1 | 2335 | 0.818 | 1.49E−21 | 0.569 | 0.529 | 0.607 |
| ANGPTL2 | 23452 | 0.795 | 9.03E−19 | 0.561 | 0.519 | 0.599 |
| GJB2 | 2706 | 0.832 | 1.49E−24 | 0.553 | 0.512 | 0.591 |
| INHBA | 3624 | 0.825 | 4.41E−22 | 0.558 | 0.518 | 0.594 |
| MMP14 | 4323 | 0.818 | 5.29E−22 | 0.563 | 0.522 | 0.6 |
| PLAU | 5328 | 0.812 | 1.96E−20 | 0.558 | 0.513 | 0.596 |
| THBS1 | 7057 | 0.855 | 1.93E−27 | 0.541 | 0.5 | 0.583 |
| THBS2 | 7058 | 0.84 | 1.88E−25 | 0.561 | 0.516 | 0.598 |
| GFPT2 | 9945 | 0.855 | 8.37E−27 | 0.571 | 0.53 | 0.605 |

TABLE 23
Subtype analysis: 45 Gene Signature

| | | ANOVA | | | |
|---|---|---|---|---|---|
| GeneID | AUC | (p value) | C-Index | LCI C-Index | UCI C-Index |
| ALPK2 | 0.754 | 4.264E−16 | 0.529 | 0.487 | 0.566 |
| ANGPTL2 | 0.794 | 1.188E−18 | 0.560 | 0.520 | 0.599 |
| BGN | 0.817 | 4.725E−21 | 0.573 | 0.530 | 0.608 |
| BICC1 | 0.795 | 2.664E−18 | 0.551 | 0.505 | 0.592 |
| CDH11 | 0.851 | 1.917E−25 | 0.557 | 0.517 | 0.593 |
| COL10A1 | 0.844 | 3.574E−25 | 0.558 | 0.512 | 0.597 |
| COL11A1 | 0.777 | 1.927E−18 | 0.574 | 0.533 | 0.611 |
| COL1A2 | 0.832 | 8.919E−23 | 0.577 | 0.533 | 0.614 |
| COL3A1 | 0.821 | 1.998E−20 | 0.590 | 0.548 | 0.628 |
| COL5A2 | 0.816 | 5.308E−23 | 0.569 | 0.528 | 0.609 |
| COL8A1 | 0.870 | 2.680E−27 | 0.566 | 0.523 | 0.603 |
| COPZ2 | 0.810 | 8.676E−20 | 0.564 | 0.526 | 0.600 |
| CTSK | 0.804 | 1.849E−19 | 0.547 | 0.506 | 0.583 |
| FAP | 0.850 | 1.374E−26 | 0.578 | 0.538 | 0.611 |
| FN1 | 0.818 | 1.828E−21 | 0.570 | 0.529 | 0.607 |
| FZD1 | 0.780 | 2.247E−15 | 0.586 | 0.546 | 0.621 |
| GFPT2 | 0.854 | 9.031E−27 | 0.572 | 0.530 | 0.611 |
| GJB2 | 0.832 | 1.666E−24 | 0.553 | 0.511 | 0.591 |
| IGFL2 | 0.773 | 2.288E−16 | 0.536 | 0.485 | 0.571 |
| INHBA | 0.825 | 4.651E−22 | 0.558 | 0.518 | 0.594 |
| ITGA5 | 0.807 | 2.079E−19 | 0.542 | 0.496 | 0.577 |
| KIF26B | 0.776 | 5.854E−15 | 0.552 | 0.511 | 0.591 |
| LOXL1 | 0.796 | 1.650E−17 | 0.550 | 0.508 | 0.590 |
| LUM | 0.823 | 1.073E−21 | 0.559 | 0.516 | 0.593 |
| MIR1245 | 0.825 | 3.785E−23 | 0.581 | 0.537 | 0.620 |
| MMP13 | 0.755 | 2.508E−14 | 0.566 | 0.523 | 0.603 |
| MMP14 | 0.815 | 1.208E−21 | 0.563 | 0.520 | 0.600 |
| MMP2 | 0.817 | 3.637E−23 | 0.553 | 0.512 | 0.590 |
| MRVI1 | 0.778 | 4.541E−17 | 0.529 | 0.486 | 0.569 |
| NKD2 | 0.802 | 1.892E−18 | 0.564 | 0.524 | 0.603 |
| NTM | 0.766 | 2.036E−15 | 0.563 | 0.524 | 0.598 |
| PLAU | 0.812 | 1.956E−20 | 0.558 | 0.512 | 0.597 |
| PMP22 | 0.808 | 1.183E−19 | 0.544 | 0.496 | 0.583 |
| POLD2 | 0.786 | 1.547E−16 | 0.589 | 0.545 | 0.626 |
| POSTN | 0.777 | 1.438E−17 | 0.569 | 0.527 | 0.602 |
| RAB31 | 0.815 | 1.676E−22 | 0.542 | 0.502 | 0.579 |
| RUNX2 | 0.784 | 1.463E−15 | 0.545 | 0.502 | 0.585 |
| SERPINF1 | 0.840 | 3.343E−24 | 0.563 | 0.522 | 0.599 |
| SFRP2 | 0.776 | 1.143E−17 | 0.569 | 0.527 | 0.605 |
| THBS2 | 0.840 | 1.511E−25 | 0.559 | 0.515 | 0.598 |
| TIMP3 | 0.787 | 6.196E−17 | 0.547 | 0.508 | 0.585 |
| TMEM200A | 0.842 | 4.891E−26 | 0.540 | 0.496 | 0.580 |
| TNFAIP6 | 0.795 | 1.600E−17 | 0.536 | 0.492 | 0.576 |
| VCAN | 0.845 | 9.655E−24 | 0.575 | 0.534 | 0.608 |
| VGLL3 | 0.800 | 5.200E−19 | 0.561 | 0.521 | 0.597 |

TABLE 14
Table to represent data in FIG. 26 - Core set analysis:
Tothill_HR_Final_Core Set Analysis_45 Gene

| | Total Delta | |
|---|---|---|
| Gene | HR | Rank |
| MMP13 | 0.251541347 | 1 |
| TNFAIP6 | 0.152036533 | 2 |
| COL3A1 | 0.121550645 | 3 |
| TIMP3 | 0.11735755 | 4 |
| NKD2 | 0.114643471 | 5 |
| KIF26B | 0.10080423 | 6 |
| FAP | 0.096789296 | 7 |
| VGLL3 | 0.095605873 | 8 |
| MMP14 | 0.095542494 | 9 |
| POLD2 | 0.092045607 | 10 |
| ALPK2 | 0.07567092 | 11 |
| RUNX2 | 0.066961369 | 12 |
| GFPT2 | 0.057289863 | 13 |
| CTSK | 0.048821463 | 14 |
| NTM | 0.045878012 | 15 |
| ITGA5 | 0.04175161 | 16 |
| FZD1 | 0.019664178 | 17 |
| COPZ2 | 0.019354845 | 18 |
| ANGPTL2 | 0.014075269 | 19 |
| LUM | 0.005097026 | 20 |
| PMP22 | 0.003327351 | 21 |
| PLAU | −0.010756737 | 22 |
| COL8A1 | −0.014008548 | 23 |
| COL1A2 | −0.015474608 | 24 |
| POSTN | −0.02055353 | 25 |
| CDH11 | −0.023093817 | 26 |
| MIR1245 | −0.031717475 | 27 |
| BICC1 | −0.03660296 | 28 |
| IGFL2 | −0.04026505 | 29 |
| MRVI1 | −0.040646101 | 30 |
| LOXL1 | −0.044712308 | 31 |
| COL11A1 | −0.051616035 | 32 |
| RAB31 | −0.06464052 | 33 |
| VCAN | −0.071803963 | 34 |
| BGN | −0.076841005 | 35 |
| MMP2 | −0.077505975 | 36 |
| TMEM200A | −0.081880796 | 37 |
| SERPINF1 | −0.083927136 | 38 |
| FN1 | −0.086805141 | 39 |
| COL5A2 | −0.091752842 | 40 |
| THBS2 | −0.115003748 | 41 |
| INHBA | −0.122749243 | 42 |
| COL10A1 | −0.122975998 | 43 |
| GJB2 | −0.147746426 | 44 |
| SFRP2 | −0.170612716 | 45 |

TABLE 15
Table to represent data in FIG. 27 - Core set analysis:
ICON7_HR_Final_Core Set Analysis_45 Gene

| Gene | Total Delta HR | Rank |
|---|---|---|
| COL11A1 | 0.230614266 | 1 |
| TNFAIP6 | 0.126558604 | 2 |
| COL8A1 | 0.113348626 | 3 |
| COL3A1 | 0.0985761 | 4 |
| THBS2 | 0.0869707 | 5 |
| POSTN | 0.082212851 | 6 |
| LUM | 0.081836943 | 7 |
| TMEM200A | 0.076763765 | 8 |
| NTM | 0.073348682 | 9 |
| ALPK2 | 0.069008899 | 10 |
| FAP | 0.063224645 | 11 |
| LOXL1 | 0.062094112 | 12 |
| GJB2 | 0.061294927 | 13 |
| RAB31 | 0.05995635 | 14 |
| INHBA | 0.056178828 | 15 |
| IGFL2 | 0.051228443 | 16 |
| TIMP3 | 0.033369809 | 17 |
| SERPINF1 | 0.024090101 | 18 |
| KIF26B | 0.0215188 | 19 |
| COPZ2 | 0.01714854 | 20 |
| POLD2 | 0.01003965 | 21 |
| BGN | 0.008585452 | 22 |
| NKD2 | 0.003216793 | 23 |
| MMP2 | −0.001775152 | 24 |
| FN1 | −0.00548725 | 25 |
| PLAU | −0.006301357 | 26 |
| ANGPTL2 | −0.006314736 | 27 |
| COL1A2 | −0.006622043 | 28 |
| RUNX2 | −0.015890395 | 29 |
| FZD1 | −0.027629684 | 30 |
| SFRP2 | −0.02829194 | 31 |
| MMP14 | −0.038025373 | 32 |
| MRVI1 | −0.038171694 | 33 |
| PMP22 | −0.04476172 | 34 |
| COL10A1 | −0.047438573 | 35 |
| COL5A2 | −0.053611989 | 36 |
| BICC1 | −0.079674476 | 37 |
| GFPT2 | −0.091136276 | 38 |
| MIR1245 | −0.110650653 | 39 |
| ITGA5 | −0.116066589 | 40 |
| MMP13 | −0.119153053 | 41 |
| VCAN | −0.127296023 | 42 |
| CDH11 | −0.133504128 | 43 |
| VGLL3 | −0.191412013 | 44 |
| CTSK | −0.207991236 | 45 |

TABLE 16
FKBP-L peptides

| | SEQ |
|---|---|
| | ID |
| SEQUENCE | NO: |
| METPPVNTIGEKDTSQPQQEWEKNLRENLDSVIQIRQQPRDPPTETLELEVSPDPASQILEHTQGAEKLV | 789 |
| AELEGDSHKSHGSTSQMPEALQASDLWYCPDGSFVKKIVIRGHGLDKPKLGSCCRVLALGFPFGSGPPEG | |
| WTELTMGVGPWREETWGELIEKCLESMCQGEEAELQLPGHSGPPVRLTLASFTQGRDSWELETSEKEALA | |
| REERARGTELFRAGNPEGAARCYGRALRLLLTLPPPGPPERTVLHANLAACQLLLGQPQLAAQSCDRVLE | |
| REPGHLKALYRRGVAQAALGNLEKATADLKKVLAIDPKNRAAQEELGKVVIQGKNQDAGLAQGLRKMFG | |
| METPPVNTIGEKDTSQPQQEWEKNLRENLDSVIQIRQQPRDPPTETLELEVSPDPASQILEHTQGAEKLV | 790 |
| AELEGDSHKSHGSTSQMPEALQASDLWYCPDGSFVKKIVIRGHGLDKPKLGSCCRVLALGFPFGSGPPEG | |
| WTELTMGVGPWREETWGELIEKCLESMCQGEEAELQLPGHTGPPVGLTLASFTQGRDSWELETSEKEALA | |
| REERARGTELFRAGNPEGAARCYGRALRLLLTLPPPGPPERTVLHANLAACQLLLGQPQLAAQSCDRVLE | |
| REPGHLKALYRRGVAQAALGNLEKATADLKKVLAIDPKNRAAQEELGKVVIQGKNQDAGLAQGLRKMFG | |
| IRQQPRDPPTETLELEVSPDPAS (referred to herein as ALM201) | 791 |
| QIRQQPRDPPTETLELEVSPDPAS | 792 |
| METPPVNTIGEKDTSQPQQEWEKNLRENLDSVIQIRQQPRDPPTETLELEVSPDPASQILEHTQGAEKLV | 793 |
| AELEGDSHKSHGSTSQMPEALQASDLWYCPDGSFVKKIVIRGHGLDKPKLGSCCRVLALGFPFGSGPPEG | |
| WTELTMGVGPWREETWGELIEKCLESMCQGEEAELQLPGHTGPPVGLTLASFTQGRDSW | |
| METPPVNTIGEKDTSQPQQEWEKNLRENLDSVIQIRQQPRDPPTETLELEVSPDPASQILEHTQGAEKLV | 794 |
| AELEGDSHKSHGSTSQMPEALQASDLWYCPDGSFVKKIVIRGHGLDKPKLGSCCRVLALGFPFGSGPPEG | |
| WTELTMGVGP | |
| METPPVNTIGEKDTSQPQQEWEKNLRENLDSVIQIRQQPRDPPTETLELEVSPDPASQILEHTQGAEKLV | 795 |
| AELEGDSHKSHGSTS | |
| METPPVNTIGEKDTSQPQQEWEKNLRENLDSVIQIRQQPRDPPTETLELEVSPDPAS | 796 |
| METPPVNTIGEKDTSQPQQEWEKNLRENLDSVIQIRQQPRDPPTETL | 797 |
| QQPRDPPTETLELEVSPD | 798 |
| QIRQQPRDPPTETLELEVSPD | 799 |
| QIRQQPRDPPTETLELEV | 800 |
| QIRQQPRDPPTETLE | 801 |
| QIRQQPRDPPTE | 802 |
| QQPRDPPTETLELEVSPDPAS | 803 |
| RDPPTETLELEVSPDPAS | 804 |
| PTETLELEVSPDPAS | 805 |
| TLELEVSPDPAS | 806 |
| RQQPRDPPTETLELEVSPD | 807 |
| RQQPRDPPTETLELEVSP | 808 |
| RQQPRDPPTETLELEVS | 809 |
| PRDPPTETLELEVSPD | 810 |
| RDPPTETLELEVSPD | 811 |

### REFERENCES

Ahmed N, Abubaker K, Findlay J, Quinn M. Epithelial mesenchymal transition and cancer stem cell-like phenotypes facilitate chemoresistance in recurrent ovarian cancer. Curr Cancer Drug Targets. 2010 May; 10(3):268-78. Review.
Ferlay, J. et al. GLOBOCAN 2012 v1.0, Cancer Incidence and Mortality Worldwide: IARC CancerBase. No. 11 [Internet]. Lyon, France: International Agency for Research on Cancer. 11, http://globocan.iarc.f (2013).
UK, C. R. Cancer Incidence for Common Cancers. Cancer Research UK 1 (2013). at <http://www.cancerresearchuk.org/cancer-info/cancerstats/incidence/commoncancers/uk-cancer-incidence-statistics-for-common-cancers>http://www.bbc.co.uk/news/uk-northern-ireland-33235308, http://www.ncri.ie/factsheets
Vaughan S, Coward J I, Bast R C Jr, Berchuck A, Berek J S, Brenton J D, Coukos G, Crum C C, Drapkin R, Etemadmoghadam D, Friedlander M, Gabra H, Kaye S B, Lord C J, Lengyel E, Levine D A, McNeish I A, Menon U, Mills G B, Nephew K P, Oza A M, Sood A K, Stronach E A, Walczak H, Bowtell D D, Balkwill F R. Rethinking ovarian cancer: recommendations for improving outcomes. Nat Rev Cancer. 2011 Sep. 23; 11(10):719-25.
McCluggage, W. G. My approach to and thoughts on the typing of ovarian carcinomas. J. Clin. Pathol. 61, 152-163 (2008).
Bowtell, D. D. The genesis and evolution of high-grade serous ovarian cancer. Nat Rev Cancer 10, 803-08, doi:10.1038/nrc2946 (2010).
Jose D G and De Kretser T. Oncogenes of human tumour cells. A review of advances in virology and molecular biology of cancer. Australas Radiol. 1984; 28(1): 43-50. PMID: 6089728.
Cohen Y, Xing M, Mambo E, et al. BRAF mutation in papillary thyroid carcinoma. J Natl Cancer Inst. 2003; 95(8):625-627.
Davies H, Bignell G R, Cox C, et al. Mutations of the BRAF gene in human cancer. Nature. 2002; 417(6892):949-954.
Xu X, Quiros R M, Gattuso P, et al. High prevalence of BRAF gene mutation in papillary thyroid carcinomas and thyroid tumor cell lines. Cancer Res. 2003; 63(15): 4561-4567. PMID: 12907632.
Hoshino R, Chatani Y, Yamori T, et al. Constitutive activation of the 41-/43-kDa mitogen-activated protein kinase signaling pathway in human tumors. Oncogene, 1999; 18(3); 813-822. PMID: 9989833.
Marshall C J. Cell signalling. Raf gets it together. Nature. 1996; 383(6596): 127-128. PMID: 8774875.
Shaul Y D and Seger R. The MEK/ERK cascade: from signaling specificity to diverse functions. Biochim Biophys Acta. 2007; 1773(8): 1213-1226. PMID: 17112607. Epub 2006 Oct. 19.
Lewis, T S, Shapiro P S, and Ahn N G. Signal transduction through MAP kinase cascades. Adv Cancer Res. 1998; 74: 49-139. PMID: 9561267.
Alberola-lla J, Forbush K A, Seger R, et al. Selective requirement for MAP kinase activation in thymocyte differentiation. Nature. 1995; 373(6515): 620-623. PMID: 7854419
Johnson G L and Vaillancourt R R. Sequential protein kinase reactions controlling cell growth and differentiation. Curr Opin Cell Biol. 1994; 6(2): 230-238. PMID: 8024815.
D'Angelo G, Struman I, Martial J, et al. Activation of mitogen-activated protein kinases by vascular endothelial growth factor and basic fibroblast growth factor in capillary endothelial cells is inhibited by the antiangiogenic factor 16-kDa N-terminal fragment of prolactin. Proc Natl Acad Sci USA. 1995; 92(14): p. 6374-6378. PMID: 7541539.
Na J, Furue M K, and Andrews P W. Inhibition of ERK1/2 prevents neural and mesendodermal differentiation and promotes human embryonic stem cell self-renewal. Stem Cell Res. 2010; 5(2): 157-169. PMID: 20675210. Epub 2010 Aug. 3.
TCGA. Integrated genomic analyses of ovarian carcinoma. Nature 2011; 474:609-15.
J. Lopez, S. Banerjee and S. B. Kaye. New developments in the treatment of ovarian cancer—future perspectives. Ann Oncol. 2013 December; 24(Suppl 10): x69-x76.
Schwarz R F, Ng C K, Cooke S L, Newman S, Temple J, Piskorz A M, Gale D, Sayal K, Murtaza M, Baldwin P J, Rosenfeld N, Earl H M, Sala E, Jimenez-Linan M, Parkinson C A, Markowetz F, Brenton J D. Spatial and temporal heterogeneity in high-grade serous ovarian cancer: a phylogenetic analysis. PLoS Med. 2015 Feb. 24; 12(2):e1001789.
Patch A M, Christie E L, Etemadmoghadam D, Garsed D W, George J, Fereday S, Nones K, Cowin P, Alsop K, Bailey P J, Kassahn K S, Newell F, Quinn M C, Kazakoff S, Quek K, Wilhelm-Benartzi C, Curry E, Leong H S; Australian Ovarian Cancer Study Group, Hamilton A, Mileshkin L, Au-Yeung G, Kennedy C, Hung J, Chiew Y E, Harnett P, Friedlander M, Quinn M, Pyman J, Cordner S, O'Brien P, Leditschke J, Young G, Strachan K, Waring P, Azar W, Mitchell C, Traficante N, Hendley J, Thorne H, Shackleton M, Miller D K, Arnau G M, Tothill R W, Holloway T P, Semple T, Harliwong I, Nourse C, Nourbakhsh E, Manning S, Idrisoglu S, Bruxner T J, Christ A N, Poudel B, Holmes O, Anderson M, Leonard C, Lonie A, Hall N, Wood S, Taylor D F, Xu Q, Fink J L, Waddell N, Drapkin R, Stronach E, Gabra H, Brown R, Jewell A, Nagaraj S H, Markham E, Wilson P J, Ellul J, McNally O, Doyle M A, Vedururu R, Stewart C, Lengyel E, Pearson J V, Waddell N, deFazio A, Grimmond S M, Bowtell D D. Whole-genome characterization of chemoresistant ovarian cancer. Nature. 2015 May 28; 521(7553):489-94.
Eleveld T F, Oldridge D A, Bernard V, Koster J, Daage L C, Diskin S J, Schild L, Bentahar N B, Bellini A, Chicard M, Lapouble E, Combaret V, Legoix-Né P, Michon J, Pugh T J, Hart L S, Rader J, Attiyeh E F, Wei J S, Zhang S, Naranjo A, Gastier-Foster J M, Hogarty M D, Asgharzadeh S, Smith M A, Auvil J M, Watkins T B, Zwijnenburg D A, Ebus M E, van Sluis P, Hakkert A, van Wezel E, van der Schoot C E, Westerhout E M, Schulte J H, Tytgat G A, Dolman M E, Janoueix-Lerosey I, Gerhard D S, Caron H N, Delattre O, Khan J, Versteeg R, Schleiermacher G, Molenaar J J, Maris J M. Relapsed neuroblastomas show frequent RAS-MAPK pathway mutations. Nat Genet. 2015 Jun. 29. doi: 10.1038/ng.3333.
Li Ren Kong, Kian Ngiap Chua, Wen Jing Sim, Hsien Chun Ng, Chonglei Bi, Jingshan Ho, Min En Nga, Yin Huei Pang, Weijie Richard Ong, Ross Andrew Soo, Hung Huynh, Wee Joo Chng, Jean-Paul Thiery, and Boon Cher Goh MEK Inhibition Overcomes Cisplatin Resistance Conferred by SOS/MAPK Pathway Activation in Squamous Cell Carcinoma. Molecular Cancer Therapeutics 2015.
Benedetti V, Perego P, Luca Beretta G, Corna E, Tinelli S, Righetti S C, Leone R, Apostoli P, Lanzi C, Zunino F. Modulation of survival pathways in ovarian carcinoma cell lines resistant to platinum compounds. Mol Cancer Ther. 2008 March; 7(3):679-87.
Lord C J, Ashworth A. Mechanisms of resistance to therapies targeting BRCA-mutant cancers. Nat Med. 2013 November; 19(11):1381-8. doi: 10.1038/nm.3369. Epub 2013 Oct. 7.
Siddiqui and Rimm. Pre-analytic variables and phospho-specific antibodies: the Achilles heel of immunohistochemistry. Breast Cancer Research 2010, 12:113
Pinhel I F, MacNeill F A, Hills M J, Salter J, Detre S, A'Hern R, Nerurkar A, Osin P, Smith I E, Dowsett M. Extreme loss of immunoreactive p-Akt and p-Erk1/2 during routine fixation of primary breast cancer. Breast Cancer Res 2010, 12:R76.
Grossi V, Peserico A, Tezil T, Simone C. p38a MAPK pathway: a key factor in colorectal cancer therapy and chemoresistance. World J Gastroenterol. 2014 Aug. 7; 20(29):9744-58. doi: 10.3748/wjg.v20.129.9744.
Kennedy R D, Bylesjo M, Kerr P, et al. Development and independent validation of a prognostic assay for stage II colon cancer using formalin-fixed paraffin-embedded tissue. J Clin Oncol 2011; 29:4620-6.
Tanney A, Oliver G R, Farztdinov V, et al. Generation of a non-small cell lung cancer transcriptome microarray. BMC medical genomics 2008; 1:20.
de Jong, S. SIMPLS: an alternative approach to partial least squares regression Chemometrics Intell Lab Syst, 18, 251-263 (1993)
Irizarry R A, Bolstad B M, Collin F, Cope L M, Hobbs B, Speed T P. Summaries of Affymetrix GeneChip probe level data. Nucleic acids research 2003; 31:e15.
Tibshirani R, Walther G, Hastie T: Estimating the number of clusters in a data set via the gap statistic. Journal of the Royal Statistical Society Series B-Statistical Methodology 63:411-423, 2001.
Ward J H. Hierarchical Grouping to Optimize an Objective Function. Journal of the American Statistical Association 1963; 58:236-244.
Pinto F R, Melo-Cristino J, Ramirez M. A Confidence Interval for the Wallace Coefficient of Concordance and Its Application to Microbial Typing Methods. PLoS One 2008; 3.
Carrico J A, Silva-Costa C, Melo-Cristino J, et al. Illustration of a common framework for relating multiple typing methods by application to macrolide-resistant Streptococcus pyogenes. J Clin Microbiol 2006; 44:2524-32.
Marisa L, de Reyniès A, Duval A, Selves J, Gaub M P, Vescovo L, Etienne-Grimaldi M C, Schiappa R, Guenot D, Ayadi M, Kirzin S, Chazal M, Fléjou J F, Benchimol D, Berger A, Lagarde A, Pencreach E, Piard F, Elias D, Parc Y, Olschwang S, Milano G, Laurent-Puig P, Boige V. Gene expression classification of colon cancer into molecular subtypes: characterization, validation, and prognostic value. PLoS Med. 2013; 10(5):e1001453. doi: 10.1371/journal.pmed.1001453. Epub 2013 May 21.
Jorissen R N, Gibbs P, Christie M, Prakash S, Lipton L, Desai J, Kerr D, Aaltonen L A, Arango D, Kruhøffer M, Orntoft T F, Andersen C L, Gruidl M, Kamath V P, Eschrich S, Yeatman T J, Sieber O M. Metastasis-Associated Gene Expression Changes Predict Poor Outcomes in Patients with Dukes Stage B and C Colorectal Cancer. Clin Cancer Res. 2009 Dec. 15; 15(24):7642-7651.
Okayama H, Kohno T, Ishii Y, Shimada Y, Shiraishi K, Iwakawa R, Furuta K, Tsuta K, Shibata T, Yamamoto S, Watanabe S, Sakamoto H, Kumamoto K, Takenoshita S, Gotoh N, Mizuno H, Sarai A, Kawano S, Yamaguchi R, Miyano S, Yokota J. Identification of genes upregulated in ALK-positive and EGFR/KRAS/ALK-negative lung adenocarcinomas. Cancer Res. 2012 Jan. 1; 72(1):100-11. doi: 10.1158/0008-5472.CAN-11-1403. Epub 2011 Nov. 11.
Virtakoivu R, Mai A, Mattila E, De Franceschi N, Imanishi S Y, Corthals G, Kaukonen R, Saari M, Cheng F, Torvaldson E, Kosma V M, Mannermaa A, Muharram G, Gilles C, Eriksson J, Soini Y, Lorens J B, Ivaska J. Vimentin-ERK Signaling Uncouples Slug Gene Regulatory Function. Cancer Res. 2015 Jun. 1; 75(11):2349-62.
Haslehurst A M, Koti M, Dharsee M, Nuin P, Evans K, Geraci J, Childs T, Chen J, Li J, Weberpals J, Davey S, Squire J, Park P C, Feilotter H. EMT transcription factors snail and slug directly contribute to cisplatin resistance in ovarian cancer. BMC Cancer. 2012 Mar. 19; 12:91. doi: 10.1186/1471-2407-12-91.
Marchini S, Fruscio R, Clivio L, Beltrame L, Porcu L, Fuso Nerini I, Cavalieri D, Chiorino G, Cattoretti G, Mangioni C, Milani R, Torri V, Romualdi C, Zambelli A, Romano M, Signorelli M, di Giandomenico S, D'Incalci M. Resistance to platinum-based chemotherapy is associated with epithelial to mesenchymal transition in epithelial ovarian cancer. Eur J Cancer. 2013 January; 49(2):520-30. doi: 10.1016/j.ejca.2012.06.026. Epub 2012 Aug. 13.

1. A method for selecting a treatment for a subject having a cancer, comprising:
(i) measuring the expression level(s) of at least 1 biomarker selected from GFPT2, TMEM200A, GJB2, MMP13, POSTN, BICC1, CDH11, MRVI1, PMP22, COL11A1, IGFL2, LUM, NTM, BGN, COL3A1, COL10A1, RAB31, ANGPTL2, PLAU, COL8A1, MIR1245, POLD2, NKD2, FZD1, COPZ2, ITGA5, VGLL3, INHBA, MMP14, VCAN, THBS2, RUNX2, TIMP3, SFRP2, COL1A2, COL5A2, SERPINF1, KIF26B, TNFAIP6, MMP2, FN1, ALPK2, CTSK, LOXL1, FAP and THBS1 the other markers in Table A or Table B in a sample of the cancer from the subject;
(ii) assessing from the expression level(s) of the at least 1 biomarker whether the sample from the subject is positive or negative for a biomarker signature comprising the at least 1 biomarker, wherein:
(a) if the sample is positive for the biomarker signature, the subject is treated with a MAPK pathway inhibitor is indicated and/or and if the sample is negative for the biomarker signature, the subject is not treated with a MAPK pathway inhibitor is not indicated; and/or
(b) if the sample is positive for the biomarker signature, the subject is treated with an EMT pathway inhibitor and is indicated and/or if the sample is negative for the biomarker signature, the subject is not treated with an EMT pathway inhibitor is not indicated; and/or
(c) if the sample is positive for the biomarker signature, the subject is not treated with an SRC pathway inhibitor and is not indicated and/or if the sample is negative for the biomarker signature, the patient is treated with an SRC pathway inhibitor is indicated; and/or
(d) if the sample is positive for the biomarker signature, the subject is treated with a taxane and is indicated and/or if the sample is negative for the biomarker signature, the subject is not treated with a taxane is not indicated.

2. The method of claim 1(a) wherein the MAPK pathway inhibitor is combined with a platinum based chemotherapeutic agent and/or an SRC pathway inhibitor.

3. A method for selecting a treatment for a subject having a cancer, comprising:
(i) measuring the expression level(s) of COL5A1 and/or THBS1 in a sample of the cancer from the subject;
(ii) assessing from the expression level(s) of COL5A1 and/or THBS1 whether the sample from the subject is positive or negative for a biomarker signature comprising COL5A1 and/or THBS1, wherein:
if the sample is positive for the biomarker signature, the subject is treated with an anti-angiogenic therapeutic agent is indicated and/or if the sample is negative for the biomarker signature, the subject is not treated with an anti-angiogenic therapeutic agent is not indicated.

4-7. (canceled)

8. The method of any previous claim 1, 2, or 3 wherein assessing whether the sample is positive or negative for the biomarker signature comprises use of classification trees.

9. The method of any previous claim 1, 2, or 3 wherein assessing whether the sample is positive or negative for the biomarker signature comprises:
(i) determining a sample expression score for the biomarker(s);
(ii) comparing the sample expression score to a threshold score; and
(iii) determining whether the sample expression score is above, equal to, or below the threshold expression score,
wherein if the sample expression score is above greater than or equal to the threshold expression score the sample is positive for the biomarker signature or and/or if the sample expression score is below less than the threshold score the sample is negative for the biomarker signature.

10. (canceled)

11. A method of treating cancer in a subject comprising administering a MAPK pathway inhibitor, an EMT pathway inhibitor, an SRC pathway inhibitor, an anti-angiogenic therapeutic agent, a taxane and/or a platinum-based chemotherapeutic agent to the subject wherein the subject is selected for treatment on the basis of using the method as claimed in any previous claim of claim 1 or 3.

12-16. (canceled)

17. A method of treating cancer in a subject comprising administering a therapeutic agent to the subject wherein
(a) if the subject cancer is positive for a biomarker signature comprising the expression level(s) of at least 1 biomarker(s) selected from GFPT2 and TMEM200A, GJB2, MMP13, POSTN, BICC1, CDH11, MRVI1, PMP22, COL11A1, IGFL2, LUM, NTM, BGN, COL3A1, COL10A1, RAB31, ANGPTL2, PLAU, COL8A1, MIR1245, POLD2, NKD2, FZD1, COPZ2, ITGA5, VGLL3, INHBA, MMP14, VCAN, THBS2, RUNX2, TIMP3, SFRP2, COL1A2, COL5A2, SERPINF1, KIF26B, TNFAIP6, MMP2, FN1, ALPK2, CTSK, LOXL1, FAP or THBS1 the other markers in Table A or Table B the therapeutic agent is a MAPK pathway inhibitor; and/or
(b) if the subject cancer is positive for the biomarker signature comprising the expression level(s) of at least 1 biomarker(s) selected from GFPT2 and TMEM200A, GJB2, MMP13, POSTN, BICC1, CDH11, MRVI1, PMP22, COL11A1, IGFL2, LUM, NTM, BGN, COL3A1, COL10A1, RAB31, ANGPTL2, PLAU, COL8A1, MIR1245, POLD2, NKD2, FZD1, COPZ2, ITGA5, VGLL3, INHBA, MMP14, VCAN, THBS2, RUNX2, TIMP3, SFRP2, COL1A2, COL5A2, SERPINF1, KIF26B, TNFAIP6, MMP2, FN1, ALPK2, CTSK, LOXL1, FAP or THBS1, the other markers in Table A or Table B the therapeutic agent is an EMT pathway inhibitor; and/or
(c) if the subject cancer is negative for the biomarker signature comprising the expression level(s) of at least 1 biomarker(s) selected from GFPT2 and TMEM200A, GJB2, MMP13, POSTN, BICC1, CDH11, MRVI1, PMP22, COL11A1, IGFL2, LUM, NTM, BGN, COL3A1, COL10A1, RAB31, ANGPTL2, PLAU, COL8A1, MIR1245, POLD2, NKD2, FZD1, COPZ2, ITGA5, VGLL3, INHBA, MMP14, VCAN, THBS2, RUNX2, TIMP3, SFRP2, COL1A2, COL5A2, SERPINF1, KIF26B, TNFAIP6, MMP2, FN1, ALPK2, CTSK, LOXL1, FAP or THBS1 the other markers in Table A or Table B the therapeutic agent is an SRC pathway inhibitor; and/or
(d) if the subject cancer is negative for the biomarker signature comprising the expression level(s) of at least 1 biomarker(s) selected from GFPT2 and TMEM200A, GJB2, MMP13, POSTN, BICC1, CDH11, MRVI1, PMP22, COL11A1, IGFL2, LUM, NTM, BGN, COL3A1, COL10A1, RAB31, ANGPTL2, PLAU, COL8A1, MIR1245, POLD2, NKD2, FZD1, COPZ2, ITGA5, VGLL3, INHBA, MMP14, VCAN, THBS2, RUNX2, TIMP3, SFRP2, COL1A2, COL5A2, SERPINF1, KIF26B, TNFAIP6, MMP2, FN1, ALPK2, CTSK, LOXL1, FAP or THBS1 the other markers in Table A or Table B the therapeutic agent is a platinum-based chemotherapeutic agent; and/or
(e) if the subject cancer is positive for a biomarker signature comprising the expression level(s) of at least 1 biomarker(s) selected from GFPT2 and TMEM200A, GJB2, MMP13, POSTN, BICC1, CDH11, MRVI1, PMP22, COL11A1, IGFL2, LUM, NTM, BGN, COL3A1, COL10A1, RAB31, ANGPTL2, PLAU, COL8A1, MIR1245, POLD2, NKD2, FZD1, COPZ2, ITGA5, VGLL3, INHBA, MMP14, VCAN, THBS2, RUNX2, TIMP3, SFRP2, COL1A2, COL5A2, SERPINF1, KIF26B, TNFAIP6, MMP2, FN1, ALPK2, CTSK, LOXL1, FAP or THBS1, the other markers in Table A or Table B the therapeutic agent is a taxane.

18. A method of treating cancer in a subject comprising administering a therapeutic agent to the subject wherein if the subject cancer is positive for a biomarker signature comprising the expression level(s) of COL5A1 and/or THBS1, the therapeutic agent is an anti-angiogenic therapeutic agent.

19-20. (canceled)

21. A method of treating cancer in a subject comprising administering a therapeutic agent to the subject, wherein:
(a) the subject has been identified as having an EMT cancer and the therapeutic agent is a MAPK pathway inhibitor, an EMT pathway inhibitor, and/or a taxane; or and/or
(b) the subject has been identified as having an EMT cancer and the therapeutic agent is an EMT pathway inhibitor; and/or
(c) (b) the subject has been identified as having a non-EMT cancer and the therapeutic agent is an SRC pathway inhibitor and/or the therapeutic agent is a platinum-based chemotherapeutic agent; and/or
(d) the subject has been identified as having a non-EMT cancer and the therapeutic agent is a platinum-based chemotherapeutic agent and/or
(e) the subject has been identified as having an EMT cancer and the therapeutic agent is a taxane.

22. (canceled)

23. The method of any of claim 1, 2, 4, 6-12, 17 or 21 or therapeutic agent for use of any of claim 14, 15, 19, or 22 wherein the therapeutic agent is a MAPK pathway inhibitor combined with a platinum-based chemotherapeutic agent.

24. A method of treating cancer in a subject comprising administering a combination of a platinum-based chemotherapeutic agent and a MAPK pathway inhibitor, wherein:
(a) the combination is used as a first line treatment; or
(b) the combination is used for a cancer identified as resistant to a platinum-based chemotherapeutic agent.

25. The combination of a platinum-based chemotherapeutic agent and a MAPK pathway inhibitor for use in the method of treating cancer, wherein:
(a) the combination is used as a first line treatment; or
(b) the combination is used for a cancer identified as resistant to a platinum-based chemotherapeutic agent claim 24.

26. (canceled)

27. The method of any one of claim 1, 2, 4, 6-12, 17, 21, or 24 or 26 or therapeutic agent for use of any of claim 14, 15, 19, or 22 or combination for use of claim 25 wherein the MAPK pathway inhibitor is selected from Table G and/or H, the EMT pathway inhibitor is selected from Table I and/or is FKBP-L polypeptide or a biologically active peptide fragment thereof, preferably ALM201, the SRC pathway inhibitor is selected from Table J and/or the taxane is Paclitaxel and/or Docetaxel.

28. The method of any one of claim 3, 5, 11, 13, or 18 or therapeutic agent for use of any of claim 14, 16, or 20 wherein the anti-angiogenic therapeutic agent is selected from Bevacizumab, Sorafenib, Nintedanib, VEGFR-2 siRNA formulated with Staramine-mPEG, a small molecule VEGF inhibitor, TRC105, Ziv-Aflibercept, CS3158, Fruquintinib, Vandetanib, Ramucirumab, Dovitinib, a hVEGF-trunc vaccine, Pazopanib, Axitinib, Regorafenib, Ponatinib, Lucitanib, Lenvatinib, Cediranib, Brivanib Alaninate, Cabozantinib, OTS102, Tivozanib, Sunitinib Malate, APX003, a Sareum VEGFR-3 program, PRS-050, X-82, CM-082, a Pieris/Syngenta Anticalin program, CTx-0357927, Linifanib, MGCD265, Dalantercept, Norcantharidin, NOX-S93, a VEGF inhibitor program, R3 antibody, AT001/r84, Muparfostat Sodium, Foretinib, Telatinib, Apatinib, AL3818, AL3810, AL8326, Icrucumab, PTC299, Plitidepsin, a vascular endothelial growth factor antisense oligonucleotide, BMS-817378, MG516, FP-1039, a VEGFR-3 monoclonal antibody, TAS-115, a TRAP based VEGFR-2 inhibitor, TLK60404, a Trap based Ar and Vegf dual inhibitor, RG7221, DCC-2701, DP-2473, DP-2514, a VEGFR-2 inhibitor, an Erbb and Vegf receptor inhibitor, Motesanib Diphosphate, Semaxanib, VGX-200, Golvatinib, GSK089, GSK089, Irinotecan, Apagin, CYC116, a FAK-FLT3-VEGFR-3 program, DMI-6867, VGX-100, A6, 1181, 4EGI-1, an Egenix cancer therapeutics program, CFAK-C4, PMX 2058, and GFB-204. Table K.

29. The method of any one of claim 1, 2, 4, 6-12, 17, 21, or 24, 26 or 27 or therapeutic agent for use of any of claim 14, 15, 19, or 22 or combination for use of claim 25 wherein the MAPK pathway inhibitor is a MEK inhibitor, optionally trametinib and/or selumetinib.

30. The method of any one of claim 1, 2, 4, 6-12, 17, 21, or 24, 26 or 27 or therapeutic agent for use of any of claim 14, 15, 19, or 22 or combination for use of claim 25 wherein the SRC pathway inhibitor is not dasatanib Dasatinib.

31. The method of any one of claim 1, 2, 4, 6-12, 17, 21, or 24, 26 or 27 or therapeutic agent for use of any of claim 14, 15, 19, or 22 or combination for use of claim 25 wherein the platinum-based chemotherapeutic agent is cisplatin.

32. The method of any one of claim 1, 2, 17, 21, or 24, therapeutic agent for use or combination for use of any previous claim, wherein the cancer is prostate cancer, ovarian cancer, breast cancer, colon cancer, or and/or lung cancer.

33. The method of claim 32 wherein the ovarian cancer is serous ovarian cancer, optionally high grade serous ovarian cancer.

34. The method, therapeutic agent for use or combination for use of claim 32 wherein the lung cancer is non-small cell lung cancer.

35. The method of any one of claim 1, 2, 17, 21, or 24, therapeutic agent for use or combination for use of any previous claim, comprising measuring the expression levels of one or more additional biomarkers selected from GFPT2, TMEM200A, GJB2, MMP13, POSTN, BICC1, CDH11, MRVI1, PMP22, COL11A1, IGFL2, LUM, NTM, BGN, COL3A1, COL10A1, RAB31, ANGPTL2, PLAU, COL8A1, MIR1245, POLD2, NKD2, FZD1, COPZ2, ITGA5, VGLL3, INHBA, MMP14, VCAN, THBS2, RUNX2, TIMP3, SFRP2, COL1A2, COL5A2, SERPINF1, KIF26B, TNFAIP6, MMP2, FN1, ALPK2, CTSK, LOXL1, FAP and THBS1. Table A and/or Table B.

36. The method of any one of claim 1, 2, 17, 21, or 24, therapeutic agent for use or combination for use of any previous claim, comprising measuring the expression levels of each of GFPT2, TMEM200A, GJB2, MMP13, POSTN, BICC1, CDH11, MRVI1, PMP22, COL11A1, IGFL2, LUM, NTM, BGN, COL3A1, COL10A1, RAB31, ANGPTL2, PLAU, COL8A1, MIR1245, POLD2, NKD2, FZD1, COPZ2, ITGA5, VGLL3, INHBA, MMP14, VCAN, THBS2, RUNX2, TIMP3, SFRP2, COL1A2, COL5A2, SERPINF1, KIF26B, TNFAIP6, MMP2, FN1, ALPK2, CTSK, LOXL1, and FAP, the biomarkers listed in Table A and/or each of GJB2, CDH11, GFPT2, COL10A1, ANGPTL2, THBS1, RAB31, THBS2, INHBA, MMP14, VCAN, PLAU, COL5A1, FAP, and FN1. of the biomarkers listed in Table B.

37. The method of any one of claim 1, 2, 17, 21, or 24, therapeutic agent for use or combination for use of any previous claim, wherein the expression score is calculated using a weight value and/or a bias value for each biomarker, and wherein the weight value and the bias value are defined for each of GFPT2, TMEM200A, GJB2, MMP13, POSTN, BICC1, CDH11, MRVI1, PMP22, COL11A1, IGFL2, LUM, NTM, BGN, COL3A1, COL10A1, RAB31, ANGPTL2, PLAU, COL8A1, MIR1245, POLD2, NKD2, FZD1, COPZ2, ITGA5, VGLL3, INHBA, MMP14, VCAN, THBS2, RUNX2, TIMP3, SFRP2, COL1A2, COL5A2, SERPINF1, KIF26B, TNFAIP6, MMP2, FN1, ALPK2, CTSK, LOXL1, and FAP, biomarker in Table A and/or each of GJB2, CDH11, GFPT2, COL10A1, ANGPTL2, THBS1, RAB31, THBS2, INHBA, MMP14, VCAN, PLAU, COL5A1, FAP, and FN1. Table B.

38. The method of any one of claim 1, 2, 17, 21, or 24, therapeutic agent for use or combination for use of any previous claim, wherein the expression level is determined at the level of RNA.

39. The method of claim 38, wherein the expression level is determined by microarray, northern blotting, RNA-seq (RNA sequencing), in situ RNA detection or nucleic acid amplification.

40. The method of any one of claim 1, 2, 17, 21, or 24, therapeutic agent for use, or combination for use of any previous claim, wherein measuring the expression levels comprises contacting the sample with a set of nucleic acid probes or primers that bind to the biomarker(s) and detecting binding of the set of nucleic acid probes or primers to the biomarker(s) by microarray, northern blotting, or nucleic acid amplification.

41. (canceled)

42. A system or test kit for selecting a treatment for a subject having a cancer, comprising:
(a) one or more testing devices for determining the expression level of at least one biomarker selected from GFPT2 and the other markers in Table A or Table B in a sample of the cancer from the subject
(b) a processor; and
(c) storage medium comprising a computer application that, when executed by the processor, is configured to:
(i) access and/or calculate the determined expression levels level(s) of the at least one biomarker in the sample on the one or more testing devices
(ii) calculate from the expression level(s) of the at least one biomarker whether the sample from the subject is positive or negative for a biomarker signature comprising the at least one biomarker; and
(iii) output from the processor the selected treatment.

43. The system or test kit of claim 42, wherein
(a) if the sample is positive for the biomarker signature, a MAPK pathway inhibitor is selected and and/or if the sample is negative for the biomarker signature a MAPK pathway inhibitor is not selected; and/or
(b) if the sample is positive for the biomarker signature, an EMT pathway inhibitor is selected and and/or if the sample is negative for the biomarker signature an EMT pathway inhibitor is not selected; and/or
(c) if the sample is positive for the biomarker signature, an SRC pathway inhibitor is not selected and and/or if the sample is negative for the biomarker signature an SRC pathway inhibitor is selected; and/or
(d) if the sample is positive for the biomarker signature an anti-angiogenic therapeutic agent is selected and and/or if the sample is negative for the biomarker signature an anti-angiogenic therapeutic agent is not selected; and/or
(e) if the sample is positive for the biomarker signature a taxane is selected and and/or if the sample is negative for the biomarker signature a taxane is not selected.

44-50. (canceled)

51. The combination of claim 25 wherein the MAPK pathway inhibitor is selected from BAL-3833, BGB-283, HM-95573, LY-3009120, RG-7304, RG-7842, Salirasib, AEZS-136, ARI-4175, ASN-003, CCT-196969, CCT-241161, CS-410, a MAP4K4 inhibitor, a RAS inhibitor, a pan-RAF Kinase inhibitor, CT-207, CT-317, a B-Raf kinase inhibitor, C-Raf inhibitor, a B-Raf and C-Raf kinase inhibitor, EBI-907, EBI-945, KO-947, LXH-254, MDC-1016, MT-477, NCB-0594, NCB-0846, NMSP-285, ON-108600, PV-103, RX-8243, STP-503, a Raf kinase inhibitor, TAK-632, TEW-0201, AIK-4, AR-00457679, CB-745, HD-001, SCH-722984, SCH-772984, a K-RAS inhibitor, a B-RAF kinase inhibitor, an ERK2 and Aurora B kinase inhibitor, ARQ-736, a K-Ras inhibitor, a pan-Raf kinase inhibitor, a TNIK inhibitor, Vemurafenib, Regorafenib, Dabrafenib, Dabrafenib mesylate, RAF-265, Encorafenib, Donafenib, NEO-100, PLX-8394, RXDX-105, TAK-580, Ulixertinib, Dabrafenib mesylate and Trametinib dimethyl sulfoxide, Sorafenib tosylate, Binimetinib and Encorafenib, Encorafenib, Dabrafenib mesylate and Panitumumab and Trametinib dimethyl sulfoxide, Donafenib, NEO-100, PLX-8394, RAF-265, RXDX-105, TAK-580, Ulixertinib, BAL-3833, BGB-283, Dabrafenib mesylate and Druvalumab and Trametinib dimethyl sulfoxide, HM-95573, Hydroxychloroquine and Sorafenib tosylate, LY-3009120, ARQ-736, CKBP-002, DP-2514, DP-3346, Fluorapacin, GDC-0879, LE-rafAON, MK-8353, NMSP-383, NMSP-730, PLX-4720, PNT-300, an Erk-1 and Erk-2 inhibitor, a tumor adaptive responses program, AEZS-131, ISIS-5132, PLX-3603, XL-281, MM-41, a GCK inhibitor, and SML-8731.

52. The method of claim 27 where the biologically active peptide fragment of FKBP-L is ALM201.

53. The combination of claim 25 wherein the MAPK pathway inhibitor is a MEK inhibitor

54. The combination of claim 25 wherein the MEK inhibitor is at least one of trametinib and selumetinib.

55. The combination of claim 25 wherein the SRC pathway inhibitor is not dasatinib.

56. The combination of claim 25 wherein the platinum-based chemotherapeutic agent is cisplatin.
